(19) United States
(12) Patent Application Publication
(10) Pub. No.: US 2006/0275900 A1
(43) Pub. Date: Dec. 7, 2006

(54) Methods for in vitro expansion and transdifferentiation of human pancreatic acinar cells into insulin-producing cells

(75) Inventors: Sharon C. Presnell, Raleigh, NC (US); Mohammad Heidaran, Cary, NC (US); Perry Haaland, Chapel Hill, NC (US); David William Scharp, Mission Viejo, CA (US); Paul Presley Latta, Irvine, CA (US); Margaret Coutts, Irvine, CA (US); Catherine Anne McIntyre, Aliso Viejo, CA (US)

Correspondence Address: VENABLE LLP, P.O. BOX 34385, WASHINGTON, DC, 20043-9998, US

(73) Assignees: Becton, Dickinson and Company, Franklin Lakes, NJ (US); Novocell, Inc., Irvine, CA (US)

(21) Appl. No.: 11/410,954

(22) Filed: Apr. 26, 2006

Related U.S. Application Data
(62) Division of application No. 10/443,732, filed on May 22, 2003.
(60) Provisional application No. 60/384,000, filed on May 28, 2002.

(51) Int. Cl. C12N 5/08; A61K 38/17

(52) U.S. Cl. 435/366; 514/12

(57) ABSTRACT

This invention relates, e.g., to a method for expanding mammalian acinar cells, comprising culturing the cells in a cell culture system comprising a cell culture medium and a cell attachment surface, under conditions wherein the acinar cells undergo a 3-4 fold expansion together with transdifferentiation into a modified cell phenotype (IP cells) showing characteristics of acinar cells and liver cells. The invention also relates to a method for transforming these IP cells to insulin-producing cells in vitro, comprising culturing the cells in a novel, defined medium. Also disclosed are suitable culture media for performing these methods, isolated cells having the phenotype of IP cells and/or produced by these methods, and kits for performing the methods.

This application claims the benefit of provisional application 60/384,000, filed May 28, 2002, which disclosure is incorporated by reference in its entirety herein.

### BACKGROUND OF THE INVENTION

1. Field of the Invention

The invention relates to compositions and methods whereby, e.g., human pancreatic acinar cells are cultured under conditions that support expansion and transdifferentiation into glandular epithelial cells and subsequently into insulin-producing cells.

2. Background Information

The potential benefits of taking insulin-producing cells from organ donors and transplanting them into insulin-dependent, Type I diabetic patients is clear. In the Edmonton clinical trials, many patients have lived free from the delivery of exogenous insulin for approximately 2 years after being transplanted with intact islets from organ donor sources. However, current technology requires two organ donor pancreata to generate a sufficient number of islets (about 1 million islets, comprised of about 1,000 cells each) to transplant into one diabetic patient for cellular therapy. Thus, there has been emphasis in the diabetes field to identify new sources of insulin-producing cells for transplantation. Many avenues are being explored, including expansion of islets after harvest and before transplantation and the generation of new islets from stem-like cells derived from the bone marrow, or from precursor cells located in the pancreas. The challenges presented by these approaches are related to maintenance of function of islets over long periods of culture, and of the relative rarity of stem-like cells that can be harnessed for insulin production from the bone marrow and pancreas. The ductular precursor stem-like cells derived from the pancreas are reported to be more efficient than bone-marrow derived cells at differentiation into insulin-producing cells, and this may reflect their site of origin (i.e., pancreas) where they are certainly exposed to many differentiation signals related to the pancreatic microenvironment. The most abundant cell type in the pancreas is the acinar cell, which comprises about 85% of the pancreas. The acinar cells serve to produce and secrete digestive enzymes and, like islet cells, arise during development from the ductular cell compartment.

There have been reports that acinar cells, when cultivated in vitro, especially under conditions of stress, can undergo a ‘transdifferentiation’ into a cell type that resembles ductular cells, as determined by expression of CK19, CK7, and carbonic anhydrase (all reputed by the authors to be markers of duct cells) (Kerr-Conte, 1996; WO 02/29010 A2), Hall et al., 1992). Furthermore, Bouwens et al. (1998) have shown in vivo, in a model of pancreatic duct ligation, that acinar cells in the ligated portion of the pancreas undergo transdifferentiation into cells with a ductular phenotype. Further work has suggested that insulin-producing cells can be produced upon further differentiation of the duct cells in the ligated portion of the pancreas. The acinar cells are also reported to be of limited survivability in primary culture, with some culture conditions leading to loss of at least 50% of cells within a week. While primary duct cells have been demonstrated in vitro to convert into insulin-producing cells under some culture conditions (e.g. Bonner Weir, 2000, U.S. Pat. No. 6,011,647), there are no reports of cells that arose from acinar cells in vitro differentiating further to produce islet-like cells.

Prior to the development of the present system, primary pancreatic acinar cells were expanded without differentiation into insulin-producing cells, either in serum-containing medium (undesirable both because of the risk and the uncertainty associated with the use of serum), or in complex serum free media formulations. Likewise, primary pancreatic acinar cells have been transdifferentiated into insulin-producing cells without expansion, producing cells with an insulin-producing phenotype in small numbers. Furthermore, it has not been previously possible to obtain insulin-producing cells in good numbers using acinar cells as starting material.

Thus, there is a need for a simple cell culture system and method for rapidly generating large numbers of cells that can further differentiate into, e.g., insulin-producing cells, through expansion and transdifferentiation of the abundant pancreatic acinar cells. Further, there is a need for a cell culture system and method for culturing and transforming such cells into insulin-producing cells. One cell culture system and related method disclosed herein allows a simple, one-step approach that generates expanded cultures that contain at least 80% intermediate progenitor cells that can give rise to insulin-producing cells. A second cell culture system and related method allows the further culturing of these intermediate progenitor cells or other glandular epithelial cells to obtain insulin-producing cells. Both IP cells and insulin-producing cells will be useful for cell-based therapies for the treatment of diseases such as diabetes.

### SUMMARY OF THE INVENTION

The present invention provides compositions and methods whereby, e.g., acinar cells can be cultivated successfully in vitro, undergoing a 3-4 fold increase in cell number over time, and giving rise to a cell population that co-expresses acinar and ductal markers early during the culture (2-3 days ex vivo), then ultimately (e.g., about 7-8 days ex vivo) acquires a modified phenotype characterized by expression of some acinar-associated genes, as well as some liver-associated genes. The genes expressed by these modified cells at about 7-8 days ex vivo include, e.g., ductular cytokeratins (CK7, CK8, CK18 and CK19), hepatic nuclear factor 1 (HNF1), alpha-1 antitrypsin, pi-glutathione s transferase (pi-GST), liver-specific (basic helix-loop-helix (bHLH) transcription factor, Thy-1, CCAAT/enhancer-binding protein (C/EBP)-alpha and C/EBP-beta. These cells exhibit little if any expression of the pancreas-associated genes carbonic anhydrase, cystic fibrosis transmembrane conductance regulator (CFTR), elastase and amylase. By “little if any” expression of a gene is meant herein that gene expression is generally undetectable under conventional methods, such as the hybridization and immunocytochemical methods described herein, but expression may be detected by extraordinarily sensitive methods, such as PCR-based analysis. This type of modified cell is referred to herein as an intermediate progenitor (“IP”) cell. The expanded/transdifferentiated acinar cells (IP cells) can be produced using a general serum-containing media, or, in a preferred method, can be produced without serum on a surface comprising one or more extracellular matrix molecules (ECMs) in the presence of one or more soluble active factors. ECMs can be presented in 2 dimensional or 3 dimensional culture systems in the presence of soluble active factors.

The IP cells generated from these cultures are expected to be useful directly in certain medical applications. For example, there is evidence that such cells may under certain conditions become functioning insulin-producing cells when implanted in diabetic patients. The cells can also be used for drug discovery and toxicity studies.

In addition, according to a further aspect of the invention, the IP cells can be cultivated further, in a serum-free medium composed of any standard serum-free base medium (DMEM:HamsF12, for example) with BSA and combinations of factors, including ECMs, small molecules, and growth factors. After 5-10 days of culture, the IP cells undergo additional steps of differentiation, culminating in the formation of cell aggregates that express pro-insulin and C-peptide. Challenge of these cultures with a high-glucose medium causes release of insulin and C-peptide into the medium, indicating the production in these cultures of functional islet-like cells.

Thus, in a first aspect, the present invention provides a cell culture system comprising a superior cell attachment surface that also stimulates cellular expansion, and a simple culture medium including effective amounts of one or more soluble active factors, or serum (e.g. fetal bovine serum), added to a base medium composition. The cell culture system will be particularly useful for primary culture of mammalian epithelial cells, particularly human epithelial cells. In a preferred embodiment the cell culture system is used for the expansion and transdifferentiation of primary acinar cells, especially human pancreatic acinar cells.

The cell attachment surface for this cell culture system is any surface to which the cells can attach and expand, including both 2 dimensional (e.g. plates, flasks, roller bottles, petri dishes, wells etc.) and 3 dimensional (e.g. scaffold) environments. Preferably the surface comprises at least one type of ECM, or a peptide fragment thereof. Cells may, in some circumstances, detach from these surfaces and form self-supporting aggregates. Suitable fragments include peptides consisting of a sequence of three of more amino acid residues that are identical to any portion of the amino acid sequence of the ECM. Such fragments can be easily made and tested by means known to those of skill in the art. Most preferably the surface is a layer of collagen I. Many other surfaces known in the art are also suitable, such as Collagen VI, Collagen IV, Vitronectin, or Fibronectin. Collagen I is preferred due to ease and cost.

The base medium to which the soluble active factors are added may be any cell culture medium appropriate for growth and differentiation of epithelial cells. These include, but are not limited to, DMEM, Hams F12, MEM, M-199 and RPMI. The general requirements for such culture media and many suitable examples are known to those of skill in the art. To this base medium is added either serum (such as fetal bovine serum), or a stabilizing protein such as bovine serum albumin (BSA) along with effective amounts of soluble active factors. The medium is preferably serum-free.

Soluble active factors for the expansion and transdifferentiation of primary pancreatic acinar cells into IP cells include growth factors such as HGF receptor activators and EGF receptor activators. Preferred soluble active factors include one or more of EGF and Transforming Growth Factor-α, IGF1, HGF, betacellulin, prolactin and gastrin 1. HGF, EGF and/or Transforming Growth Factor-α are particularly preferred. Also preferred is the combination of IGF1 and betacellulin.

In one particularly preferred embodiment, the base medium contains a 1:1 mixture of DMEM and Hams F12. The base medium is completed with the addition of glutamine to a final concentration of ˜4 mM, insulin (˜0.1-10 μg/ml, preferably ˜0.01 mg/ml), transferrin (˜0.5-10 μg/ml, preferably ˜0.0055 mg/ml), selenium (˜0.25-5.0 ng/ml, preferably ˜0.0067 μg/ml of sodium selenite), and Epidermal Growth Factor (EGF) (˜1-20 ng/ml, preferably ˜10 ng/ml); this medium is hereafter referred to as pancreatic cell medium, or PCM. To this base medium formulation, up to ˜20% Fetal Bovine Serum (or other serum), preferably between ˜10-˜15% fetal bovine serum, most preferably about 10% or up to about 15% fetal bovine serum) may be added, or, to create a serum-free culture environment, the following components are added in place of serum: heat-inactivated bovine serum albumin (0.1-2%), Hepatocyte growth factor (HGF) (1-20 ng/ml), and/or Transforming Growth Factor Alpha (TGFα) (1-10 ng/ml). In addition, the medium may contain Betacellulin (0.5-20 ng/ml), Gastrin 1 (0.05-10 ng/ml), Prolactin (1.0-10 ng/ml), and/or IGF-1 (5-100 ng/ml). In particular formulations, greater or lesser amounts of these components may be added in order to achieve a formulation that is effective in supporting the expansion and transdifferentiation of the cells. Persons of skill in the art will appreciate that determining effective amounts of the components will require no more than routine experimentation.

By the use of this attachment surface and medium, the expansion and transdifferentiation of primary pancreatic cells with the desired phenotype is simplified greatly.

In a particularly preferred embodiment, the cell culture system is a combination of collagen I coated tissue culture surface (presented in a 2 dimensional or 3 dimensional form) and a serum-free medium containing BSA, insulin, transferrin, selenium, Hepatocyte growth factor (HGF), Epidermal Growth Factor (EGF) and Transforming Growth Factor Alpha (TGFA).

The cell culture system enables superior attachment in vitro of primary pancreatic epithelial cells for adherent culture compared to prior methods, while creating a cellular environment that promotes expansion of the epithelial component of primary pancreatic cultures with concomitant transdifferentiation of the acinar cells present in the starting material into IP cells, while minimizing emergence of undesired fibroblasts. Advantages of this culture system are ease of construction, few components needed, and that all components are readily available and easily used in the required manner.

The components of this aspect of the invention may be conveniently packaged in the form of a kit. The kit may include, for example, 1) a cell culture medium such as DMEM: 2) a serum-free medium supplement containing BSA, insulin, transferrin, selenium, HGF, EGF and TGFA, in suitable amounts to yield the concentrations noted above in the completed medium; and 3) at least one collagen I coated substrate, such as a vessel for tissue culture (e.g., dish(es) with at least one collagen-1 coated tissue culture surface), or collagen-1 coated inserts for use in culture dishes or other laboratory ware. The kit may also optionally include a tissue culture dish or other cell culture accessories and additional reagents that may be required to carry out epithelial cell culture and differentiation.

Culture systems consisting of scaffolds, collagen coated flasks or other vessels and serum-free base medium may be packaged along with the soluble active factors as a separate vial that would be added to the culture medium just prior to use. The active factor combination can be added to a variety of base media to accomplish the same end, e.g., growth and differentiation of primary pancreatic acinar cells in vitro. Such culture systems should also be useful for other cell types, particularly glandular epithelial cells derived from other organs and tissues, including those from liver, pancreas, intestine, prostate, and breast.

The collagen I surface provides superior cell attachment (thereby increasing the number of cells that adhere during initial culture and thus enhancing culture efficiency), while the collagen I and the combination of soluble active factors (e.g., HGF, TGFA and EGF) promote continued proliferation of cells over time, leading to an increase in cell number above what has been previously reported for primary pancreatic acinar cells. Furthermore, the expansion of the acinar cells is accompanied by a transdifferentiation in the majority of cells to an IP phenotype, which is potentially a therapeutically useful cell phenotype for the treatment of diseases such as diabetes. This likely occurs due to convergence of the intracellular signaling pathways associated with collagen I, HGF, TGFA and EGF, creating a synergistic response.

The cell culture system of the present invention has unexpected advantages over systems previously in use. Collagen I, IV, VI, Vitronectin and Fibronectin were expected to enhance cell attachment. However, other extracellular matrix molecules that yielded equivalent attachment of cells during the initial 18 hours of culture did not promote consistent growth of the cells over time in the serum-free medium containing HGF/EGF/TGFA. The most efficient and cost-effective method of achieving cell expansion AND differentiation into IP cells is to utilize a collagen-I surface and a medium containing reduced serum (preferably less than 20%, more preferably less than 15%, 10%, or 5%, most preferably 2%).

Another aspect of the invention is a method for culturing mammalian epithelial cells comprising adding said cells to the cell culture system described above, and maintaining them at suitable temperature and atmospheric conditions. By “mammalian epithelial cell” is meant any cell of a tissue or organ with an epithelial cell phenotype, defined by the presence of expression of cytokeratins and often through the presence of markers that suggest a tissue-specific function (i.e., epithelial cells of the skin make keratin, epithelial cells of the intestine make mucin, epithelial cells of the prostate make PSA). In a preferred embodiment, the cells are primary pancreatic cells, particularly human pancreatic cells. Suitable temperature for mammalian cells is usually in the range of about 37° C., but may be varied somewhat according to cell type. The atmosphere can be ordinary air, or other specialized mixtures of gasses suitable for maintaining cells, as will be familiar to persons of skill in the art. Expansion of pancreatic acinar cells can be maximized by decreasing the oxygen tension in the culture atmosphere to less than 21%, while transdifferentiation to IP cells can be enhanced by increasing oxygen tension to greater than 5%. A preferred range of oxygen tension is between about 5% and about 21%.

In a second aspect, the invention also provides methods and compositions for transforming glandular epithelial cells that have acquired expression of markers characteristic of an intermediate progenitor (IP) phenotype as described above into insulin-producing cells. By “glandular epithelial cell” is meant an epithelial cell that is a component of a gland. Glands are tissues that have a specific function related to secretion of key molecules—most organs in the body have glandular function (liver, intestine, pancreas, prostate, breast, pituitary, adrenal, kidney) whereby they produce and release hormones, digestive enzymes, or other life-essential fluids. Glandular epithelial cells from endoderm-derived organs (e.g., liver, intestine, pancreas) share many characteristics, including the ability to express many of the same genes. Particularly preferred are glandular epithelial cells from pancreas, for example acinar cells. As used herein, the terms “express” and “expression” generally refer to nucleic acids (e.g., mRNAs) or to protein gene products that are detectable by standard immunocytochemical methods.

In this aspect, the invention provides a second cell culture system comprising a cell attachment surface and a culture medium that supports and promotes the transformation of glandular epithelial cells into insulin-producing cells. The cell attachment surface is similar to and may be identical to the attachment surface for expanding primary pancreatic acinar cells. It may be presented in the form of a flat surface coated on a vessel or in the form of a scaffold or other surface adapted for cell culture. It can be comprised of, or coated with, any composition that is capable of maintaining cells or supporting cell growth. In a preferred embodiment, it comprises at least one ECM, such as Collagen I, Collagen VI, Collagen IV, Vitronectin or Fibronectin. In a particularly preferred embodiment, the cell attachment surface is Collagen-I.

In this aspect, the invention provides a further culture medium comprising at least one differentiation promoting factor (“DPF”) that promotes the transformation of glandular epithelial cells into insulin producing cells. The DPFs for the transformation of glandular epithelial cells into insulin producing cells can be one or more of Activin A, acidic FGF, basic FGF, C-Natriuretic Peptide (CNP), Calcitonin Gene Related Peptide, Cholera Toxin B Subunit, Dexamethasone, Gastrin-Releasing Peptide, Glucagon-like Peptide-1 (GLP-1), Glucose, IGF1, IGF2, Insulin, Laminin, LIF, Met-Enkephalin, PDGFAA+PDGFBB, Prolactin, Sonic Hedgehog, Substance P, TGF-alpha, Trolox (alpha-tocopherol derivative), or VEGF. Preferred concentrations in culture medium of each of these 23 DPFs are listed in Table 1. Although in some cases one DPF is sufficient, preferably two or more factors are used. As many as all 23 of the factors may be used.

TABLE 1

| | Concentration |
|---|---|
| | (micrograms/ |
| Substance | mL) |
| Activin A (human, recombinant) | 0.0005 |
| CGRP alpha, (Calcitonin Gene Related Peptide, rat) | 0.1905 |
| C natriuretic peptide) (human, porcine, rat: frag 32-53) | 0.10985 |
| (CNP) | |
| Cholera Toxin B Subunit, recombinant | 0.0125 |
| DEXamethasone (9 alpha-fluoro-16alpha- | 0.002 |
| methylprednisolone)(hydrocortisone analogue) | |
| FGF acidic (aFGF = FGF1), Recombinant Human | 0.0025 |
| GLP-1 (7-36) amide, human (Glucagon-Like Peptide | 0.033 |
| 1) | |
| Glucose (base should be l0ow; 0.9 ug/ml) | 1.08 |
| Insulin, human (low [ ] in base media 1 ug/ml) | 9.5 |
| LIF, human (leukemia inhibitory factor, human) | 0.0025 |
| PDGF AA + PDGF BB MIX | 0.005 |
| TGF alpha | 0.001 |
| Prolactin (human, recombinant)(a plasma growth | 0.0012 |
| hormone) | |
| Trolox (soluable Vitamin E) (C14H18O4) | 0.625 |
| GRP (Gastrin Releasing Peptide, Human) | 0.143 |
| IGF-1, recombinant human | 0.0025 |
| IGF-2, recombinant human | 0.0025 |
| Laminin | 2.25 |
| Met-Enkephalin (tyr-gly-glyl-phe-met) | 0.003 |
| Sonic Hedgehog (mouse, recombinant) | 0.025 |
| Substance P (full length) (H1875 is frag 1-4) | 5 |
| FGFb (=FGF2), human Recombinant | 0.0025 |
| VEGF | 0.0025 |

In a preferred embodiment of this aspect of the invention, the culture medium comprises at least one (or as many as all 10) of the following differentiation promoting DPFs:.C-Natriuretic Peptide (CNP), Calcitonin Gene Related Peptide, Cholera Toxin B Subunit, Dexamethasone, Gastrin-Releasing Peptide, Laminin, Met-Enkephalin, PDGFAA+PDGFBB, Sonic Hedgehog, and Substance P.

In a preferred embodiment, the culture medium that promotes the transformation of glandular epithelial cells into insulin producing cells consists of a 1:1 mixture of DMEM and Hams F12 plus the components listed in Table 2. This medium is sometimes referred to herein as “Media or Medium G9.”

TABLE 2

| | Factor | Substance | Concentration (Final) ug/ml |
|---|---|---|---|
| 1 | ANP | Atrial Natriuretic | 0.1530 |
| | | Peptide, Rat (28 amino | |
| | | acids) | |
| 2 | CCK-8-frag-amide | CCK8, Fragment 26-33 | 0.0250 |
| | | Amide | |
| | | (Cholecystokinin) | |
| | | (Asp-Tyr(SO3H)-Met- | |
| | | Gly-Trp-Met-Asp-Phe- | |
| | | NH2) | |
| 3 | Caerulin sulfate | Caerulein (Pyr-Gln- | 0.0300 |
| | | Asp-Tyr(SO3H)-Thr- | |
| | | Gly-Trp-Met-Asp-Phe- | |
| | | NH2 | |
| 4 | Cholera Toxin-B | Cholera Toxin B | 0.0125 |
| | | Subunit, recombinant | |
| 5 | Dex | DEXamethasone (9 | 0.0020 |
| | | alpha-fluoro-16alpha- | |
| | | methylprednisolone)(hydrocortisone | |
| | | analogue) | |
| 6 | FGF-7 | FGF7 (KGF) | 0.0025 |
| 7 | GLP-1 | GLP-1 (7-36) amide, | 0.0330 |
| | | human (Glucagon-Like | |
| | | Peptide 1) | |
| 8 | GRP | GRP (Gastrin | 0.1430 |
| | | Releasing Peptide, | |
| | | Human) | |
| 9 | Gastrin-1 | Gastrin I Human | 0.0000 |
| 10 | Glucose | Glucose (base should | 1.0800 |
| | | be l0ow; 0.9 ug/ml) | |
| 11 | HGF | Hepatocyte Growth | 0.0025 |
| | | Factor (HGF) | |
| | | recombinant | |
| 12 | IGF-1 | IGF-1, recombinant | 0.0025 |
| | | human | |
| 13 | IGF-2 | IGF-2, recombinant | 0.0025 |
| | | human | |
| 14 | Insulin | Insulin (low [ ] in base | 9.5000 |
| | | media 1 ug/ml) | |
| 15 | Leu-Enkephalin | Leu-Enkepthalin (tyr- | 0.0030 |
| | | gly-gly-phe-leu) | |
| 16 | Nicotinamide | Nicotinamide | 610.0000 |
| 17 | PTHRP-frag-1-34 | pT II RP (Parathyroid | 0.2060 |
| | | Hormone Related | |
| | | Peptide (1-34), human) | |
| 18 | Progesterone | Progesterone | 0.0030 |
| 19 | Prolactin | Prolactin (human, | 0.0012 |
| | | recombinant)(a plasma | |
| | | growth hormone) | |
| 20 | Retinol acetate | Retinoic Acid (Vitamin | 0.0250 |
| | | A) | |
| 21 | SHH | Sonic Hedgehog | 0.0250 |
| | | (mouse, recombinant) | |
| 22 | Sodium selenite | Selenium (Selenous | 0.0250 |
| | | Acid, Na salt) | |
| 23 | Soybean trypsin inhibitor | Trypsin Inhibitor, | 0.5000 |
| | | soybean (type I-S) | |
| 24 | TGF-beta sRII | TGF beta sRII (soluable | 0.0050 |
| | | receptor type 2) | |
| 25 | Transferrin | transferrin | 2.7500 |
| 26 | Trolox | Trolox (soluable | 0.6250 |
| | | Vitamin E) (C14H18O4) | |
| 27 | VIP | Vasoactive Intestinal | 0.0665 |
| | | Peptide (VIP), human | |
| 28 | aFGF | FGF acidic (aFGF = FGF1), | 0.0025 |
| | | Recombinant | |
| | | Human | |
| 29 | bFGF | FGFb (=FGF2), human | 0.0025 |
| | | Recombinant | |
| 30 | n-Butyric acid | n Butyric Acid, Sodium | 4.5400 |
| | | Salt | |

The components of this aspect of the invention may also be conveniently packaged in the form of a kit. The kit may include, for example, 1) a cell culture medium such as DMEM, Hams F12, or a combination thereof, 2) a serum-free medium supplement containing: BSA and the DPFs Activin A, acidic FGF, basic FGF, C-Natriuretic Peptide (CNP), Calcitonin Gene Related Peptide, Cholera Toxin B Subunit, Dexamethasone, Gastrin-Releasing Peptide, Glucagon-like Peptide-1 (GLP-1), Glucose, IGF1, IGF2, Insulin, Laminin, LIF, Met-Enkephalin, PDGFAA+PDGFBB, Prolactin, Sonic Hedgehog, Substance P, TGF-alpha, Trolox (alpha-tocopherol derivative), or VEGF, or two or more of these components in combination, in suitable amounts to yield the concentrations noted in Table 1 in the completed medium; and 3) tissue culture dish(es) with at least one collagen-1 coated tissue culture surface (or collagen-1 coated inserts for use in culture dishes or other laboratory ware). The kit may also optionally include a tissue culture dish and/or other cell culture accessories and additional reagents that may be required to carry out epithelial cell culture and differentiation. In other embodiments, the kit may contain any of the media or media components discussed herein.

Culture systems consisting of scaffolds, collagen coated flasks or other vessels and serum-free base medium may be packaged along with the DPF(s) as a separate vial that would be added to the culture medium just prior to use. The DPF combination can be added to a variety of base media to accomplish the same end, e.g., growth and differentiation of primary pancreatic acinar cells in vitro. Such culture systems may also be useful for other cell types, particularly other epithelial cells derived from glandular tissues, including those from liver, pancreas, intestine, prostate, and breast.

The invention also provides a method for converting glandular epithelial cells into insulin-producing cells comprising culturing the glandular epithelial cells in the cell culture system described above. The method may further comprise removing the culture medium from the cell culture, re-feeding the cell culture with a serum-free medium with glucose, and measuring proinsulin production C-peptide production, or insulin release.

Furthermore, the invention provides an isolated population of insulin-producing cells containing cytoplasmic granules with immunodetectable proinsulin, insulin, and/or c-peptide that is derived from a population of cells of which a subset of cells expressed at least one marker associated with IP cells (e.g., expressed some acinar-associated genes, as well as some liver-associated genes, including, e.g., ductular cytokeratins (CK7, CK8, CK18 and CK19), HNF1, alpha-1 antitrypsin, pi-glutathione s transferase (pi-GST), liver-specific bHLH transcription factor, Thy-1, C/EBP-alpha and C/EBP-beta, and expressed little if any of the pancreas-associated genes carbonic anhydrase, cystic fibrosis transmembrane conductance regulator (CFTR), elastase and amylase).

By an “isolated” cell or population of cells is meant herein that the cell or cell population is removed from its original environment (e.g., the natural environment if it is naturally occurring), and isolated or separated from at least one other component with which it is naturally associated. For example, a naturally-occurring cell present in its natural living host is not isolated, but the same cell, separated from some or all of the coexisting materials in the natural system, is isolated. Such cell or cell populations could be part of a cell culture or cell population, and still be isolated in that such culture or population is not part of its natural environment.

In one preferred embodiment, the insulin-producing cells are derived from glandular epithelial cells obtained from mammalian pancreas, such as primary acinar cells.

The data disclosed in the examples below are generated from freshly isolated human pancreatic cells. The expansion of primary human pancreatic cells in these conditions produces cultures with a mixed epithelial IP phenotype, suitable for in vitro studies of IP cells for a variety of purposes, and suitable for transplantation in vivo for cell therapy for the treatment of diseases such as diabetes. The IP cells generated by these methods may also be useful in the study of pancreatic cell biology, as normal controls in the study of pancreatic epithelial cancers, and to test the effects of drugs/compounds on normal pancreatic epithelial cells (ductal or acinar). Furthermore, the cells may be further cultured to yield insulin-producing cells as demonstrated below.

### BRIEF DESCRIPTION OF THE DRAWINGS

FIGS. 1A-D show microscopic images after treatment of starting material with antibodies to amylase (FIG. 1A), insulin (FIG. 1B), and CK19 (FIG. 1C) and the composition of the cell pellet of freshly isolated primary human pancreatic cells (FIG. 1D).

FIG. 2 shows growth curves constructed from primary human pancreatic cultures grown in commercial medium (with serum) or in the described pancreatic cell medium (PCM) with serum.

FIG. 3 shows a comparison of cell expansion in the base medium composition described vs. base medium+soluble growth factors (serum-free formula) vs. base medium+fetal bovine serum.

FIGS. 4A-B shows the effect of different culture surfaces on total cell number (FIG. 4A) and cell phenotype (FIG. 4B) after expansion.

FIGS. 5A-B show a comparison of cell phenotype after expansion in serum-containing (5A) and serum-free (5B) medium containing all soluble active factors.

FIG. 6 shows high power images of cell cultures expanded in various conditions, including serum-free base media supplemented with 3 soluble active factors, HGF, EGF & TGFA. Note epithelial morphology.

FIG. 7 shows a demonstration of growth of IP cells on ECM-coated surfaces as determined by metabolic activity assay over time. Note superior growth when Collagen I surface is combined with the media formulation described herein, yielding results superior to the combination of Matrigel and commercial media with serum.

FIG. 8A (upper left) shows expression of amylase by acinar cells after two days of culture (red staining), FIG. 8B (lower left) shows expression of CK19 (green staining), FIG. 8C (right) shows an overlay of the two images, showing co-expression (yellow) in a large proportion of cells.

FIG. 9 shows changing phenotype of primary acinar cells in culture over 5 days. Amylase is red, CK19 is green. Note appearance of yellow (amylase+CK19) on Day 2 and 3.

FIGS. 10A and 10B show primary human pancreatic cells that were expanded in serum-containing medium on Collagen I coated surface. Images were analyzed to determine total cells (FIG. 10A, blue nuclei) and total positive cells (FIG. 10B, blue nuclei surrounded by green staining for CK19).

FIG. 11 shows light microscopic (200×) appearance of pancreatic acinar cells cultured on a collagen I surface with all DPFs (Activin A, 0.5 ng/ml; acidic FGF, 2.5 ng/ml; basic FGF, C-Natriuretic Peptide (CNP), 0.11 μg/ml; Calcitonin Gene Related Peptide, 0.19 μg/ml; Cholera Toxin B Subunit, 12.5 ng/ml; Dexamethasone, 0.002 μg/ml; Gastrin-Releasing Peptide, 0.143 μg/ml; Glucagon-like Peptide-1 (GLP-1), 0.033 μg/ml; Glucose, 1.08 μg/ml; IGF1, 0.0025 μg/ml; IGF2, 0.0025 μg/ml; Insulin, 9.5 μg/ml; Laminin, 2.25 μg/ml; LIF, 0.0025 μg/ml; Met-Enkephalin, 0.0030 μg/ml; PDGFAA+PDGFBB (0.0050 μg/ml: 0.0025 μg/ml of PDGFAA+0.0025 μg/ml PDGFBB); Prolactin, 0.0012 μg/ml; Sonic Hedgehog, 0.025 μg/ml; Substance P, 5.0 μg/ml; TGF-alpha, 0.0010 μg/ml; Trolox (alpha-tocopherol derivative), 0.625 μg/ml; and VEGF, 0.0025 μg/ml).

FIG. 12A (top right panel) shows immunocytochemical analysis with CK19 antibodies (green).

FIG. 12B (lower right panel) shows immunocytochemical analysis with C-peptide antibodies (red).

FIG. 12C (left panel) shows an overlay image demonstrating the colocalization of CK19 and C-peptide (orange). Blue portions are DAPI stained nuclei.

FIG. 13A shows insulin release upon glucose challenge in IP cells that have not been detached and relocated (subcultured) during the growth and differentiation process.

FIG. 13B shows insulin release upon glucose challenge in IP cells that have been subcultured according to Example 10.

FIG. 13C shows C-peptide release upon glucose challenge in IP cells that have not been subcultured according to Example 10.

FIG. 14 shows the Insulin/DNA ratio in subcultured and nonsubcultured cells that are treated with Combinations 1, 2 and 3 of DFP media, as described in Example 11.

FIG. 15 shows insulin release in response to base level glucose (5 mm) and a glucose challenge (22 mm) over 10 days of culture in PCM and DPF media, as described in Example 13.

FIG. 15A shows insulin release in response to base level glucose (5 mm) and a glucose challenge (22 mm) over 14 days of culture in PCM and DMG9 media, as detailed in Example 14.

FIG. 16 is a graphical representation of the characteristics of the 17 classes of genes shown in Table 6, as indicated in the last column of the Table, as detailed in Example 14.

### DETAILED DESCRIPTION OF THE INVENTION

In describing preferred embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. Each reference cited here is incorporated by reference as if each were individually incorporated by reference.

The following abbreviations are used:

BSA: bovine serum albumin

BMP Bone Morphogenetic Protein

bHLH: basic helix loop helix

DMEM: Dulbecco's Modified Eagle's Medium

TGFβ1: Transforming Growth Factor β1

ECM: extracellular matrix molecules; naturally occurring proteins produced by cells of a tissue that provide structural support as well as a source of cellular signals related to adhesion. Examples are collagen, vitronectin, fibronectin, laminin.

EGF: Epidermal Growth Factor

Hams F12: Ham's Nutrient Mixture F12

HGF: Hepatocyte growth factor

HNF-1: Hepatic nuclear factor 1

IGF1: Insulin-like growth factor 1

IGF-II: Insulin-like growth factor 2

IP cells: Intermediate progenitor cells derived from an epithelial cell, such as, e.g., a pancreatic acinar cell or a liver cell, wherein the derived cells express some acinar-associated genes, as well as some liver-associated genes, including, e.g., cytokeratins (CK7, CK8, CK18 and CK19), HNF1, alpha-1 antitrypsin, pi-glutathione s transferase (pi-GST), liver-specific bHLH transcription factor, Thy-1, C/EBP-alpha and C/EBP-beta, and express little if any of the pancreas-associated genes carbonic anhydrase, cystic fibrosis transmembrane conductance regulator (CFTR), elastase and amylase).

PDGF-A: Platelet derived growth factor alpha

PDGF-B: Platelet derived growth factor beta

TGFA, TGF-α Transforming Growth Factor α

As used herein, the term “culture system” is intended to mean a system for growing and/or differentiating cells in culture, which comprises a cell attachment surface, preferably one that also stimulates cellular expansion, and a culture medium, which includes effective amounts of one or more factors, or serum (e.g. fetal bovine serum), added to a base medium composition.

When referring to active soluble factors and DPFs herein, “effective amount” means an amount that either alone or in combination with other included factors is effective in promoting either expansion and differentiation into IP cells, or into insulin-producing cells, as applicable.

### EXAMPLES

I. Expansion and Transdifferentiation of Primary Acinar Cells into Glandular Epithelial Cells (Culture Phase I)

Materials and Methods:

Starting Material: Primary human pancreatic acinar cells are collected as waste from standard COBE gradient preparation of islet cells for transplantation (Lake et al., 1989). After density gradient centrifugation, the islets are present as a layer between 1.063 density and 1.10 density, and the remaining cells are collected as the pellet that sediments to the bottom of the gradient based on density. Approximately 48 hours after collection of the cells at the transplant center are received by the inventors in non-tissue-culture treated polystyrene flasks and are suspended in RPMI+10% fetal calf serum at a density of approximately 2.0 million cells/ml. Cell number and viability is assessed by trypan blue exclusion and enumeration on a hemacytometer by light microscopic observation.

Phenotypic Evaluation of Starting Material. A preparation of starting material was formalin fixed and paraffin-embedded as a cell pellet about 24 hours after initial harvest of the pancreatic cells. Paraffin sections were prepared, placed on slides, and subjected to immunocytochemical analysis with antibodies to insulin (Biogenex, San Ramon, Calif.), CK19 (Biogenex), and Amylase (Biogenex). A minimum of (3) sections per sample were assessed with each marker. All antibody staining was carried out according to the manufacturer's suggestion with pre-diluted commercial antibodies. For CK19, a 3 minute treatment with pepsin enzyme (Biogenex) preceded the blocking step for the purpose of antigen retrieval. Briefly, the sections were rehydrated through graded ethanols, followed by a 15 minute incubation in phosphate buffered saline (PBS) without calcium and magnesium. Protein Blocker (Biogenex) was added for 30 minutes prior to addition of primary antibodies. After (3) 5-minute washes, biotinylated secondary antibody (Biogenex) was added at a 1:100 dilution and sections were incubated for 30 minutes at room temperature. After (3) 5-minute washes, Alexa488 or Alexa-596-conjugated StreptAvidin (Molecular Probes, Eugene, Oreg.) was added for fluorescent visualization. For each slide, a minimum of (3) 200× images were captured using a Nikon fluorescent microscope fitted with a SPOT camera (Diagnostic Systems, Inc., Webster, Tex.). The images were assessed quantitatively using image analysis software (MetaMorph/Universal Imaging Corporation, Downington, Pa.) to determine relative fraction of insulin-positive, CK19+, and amylase+ cells. Insulin+ cells are the beta cells of the islets, CK19+ cells are the primary ductal cells, and amylase+ cells are the acinar cells (see Example 1).

### Example 1

### Characterization of Cell Culture Conditions

A. Serum-Free Medium

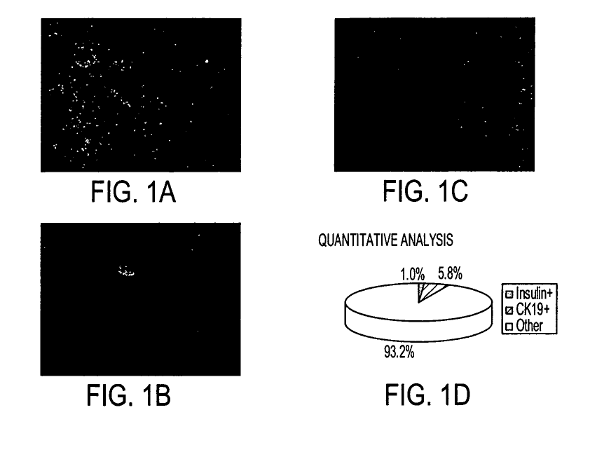

Freshly isolated primary human pancreatic cells were collected as a pellet from a COBE cell separator, fixed in formalin, paraffin-embedded, sectioned, and analyzed with antibodies to amylase, CK19, and Insulin. Images (FIGS. 1A and 1B) were collected on a Universal Imaging System (Universal Imaging Corporation) and analyzed with MetaMorph Software. This cell pellet (FIG. 1C) was comprised of 1.0% insulin+ cells (beta cells of the islet), 5.8% CK19+ cells (primary ductal cells), and 93.2% amylase+ and unlabeled (acinar cells and other cell types).

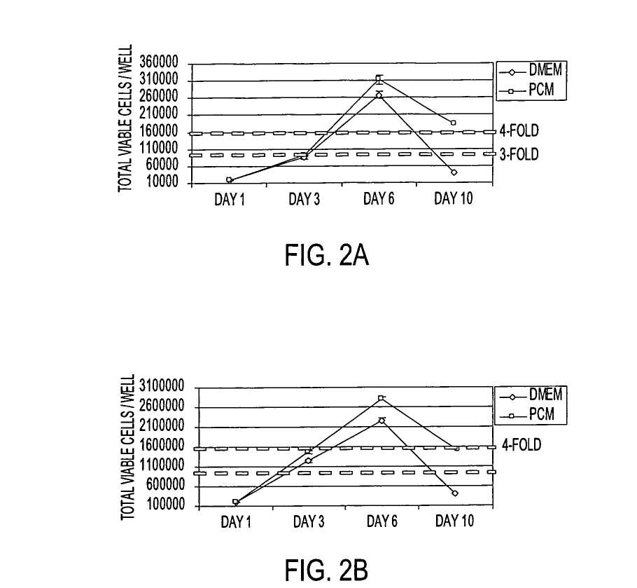

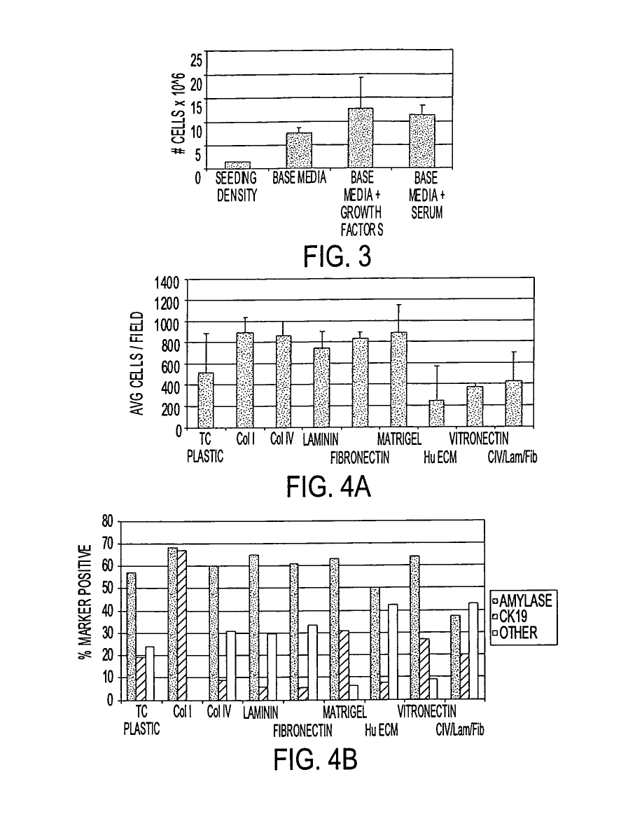

Primary human pancreatic cells were then seeded at 10⁴ or 10⁵ cells/cm² onto tissue culture treated polystyrene in either DMEM commercial medium plus 10% fetal bovine serum or in PCM plus 10% fetal bovine serum. Replicate cultures were harvested at 3 day intervals via trypsinization and live cells (as determined by trypan blue exclusion) and enumerated on a hemocytometer. The results (shown in FIG. 2) demonstrate that the (serum-containing) medium formulation described herein is superior to commercial media formulation for growth and maintenance of primary pancreatic cells. FIG. 3 compares the results of expanding the cells for 6 days in base medium, base medium plus all of the soluble active factors [HGF, ˜1-˜20 ng/ml, preferably ˜5.0 ng/ml; TGFA, ˜1-˜10 ng/ml, preferably ˜2 ng/ml; Betacellulin, ˜0.5-˜20 ng/ml, preferably ˜10 ng/ml; Gastrin 1, ˜0.05-˜10 ng/ml, preferably ˜0.06 ng/ml; Prolactin, ˜1.0-˜10 ng/ml, preferably ˜2.4 ng/ml; and IGF1, ˜5-˜100 ng/ml, preferably ˜5 ng/ml] and base medium plus 10% serum. The serum-free media formulation meets/exceeds expansion provided by media+serum.

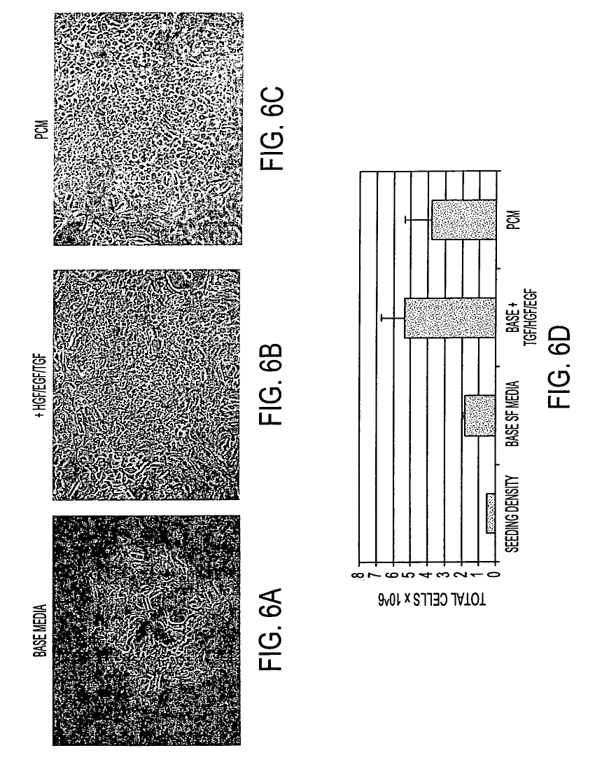

The cell expansion experiment was repeated essentially as above, except that the base medium was supplemented with only three of the soluble active factors: TGF, HGF, and EGF. FIG. 6D compares the results of expanding the cells in the various media; FIGS. 6A, 6B and 6C show high power images of the cell cultures expanded under the various media conditions.

B. ECM Surfaces

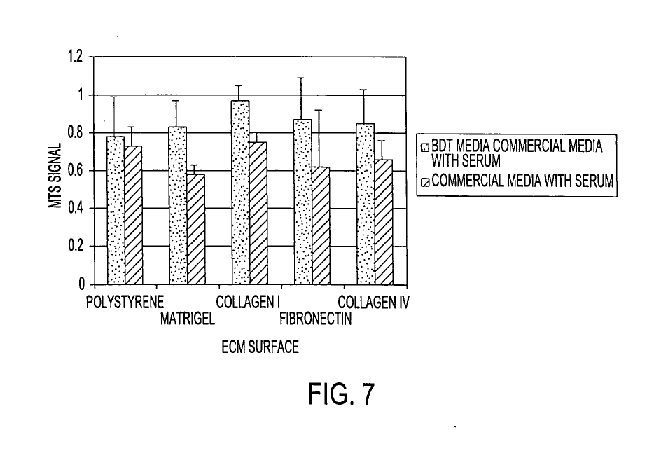

The attachment of primary human pancreatic cells was evaluated by counting the number of attached cells vs. the number of cells initially seeded on a panel of ECM surfaces comprised of Collagen I (1 μg/cm²), Fibronectin (3 μg/cm²), Laminin (2 μg/cm²), Vitronectin (1 μg/cm²), Matrigel (1 μg/cm²), Human ECM (1 μg/cm²), or Poly-D-Lysine (3 μg/cm²). In one condition, a mixture of Collagen IV, Laminin, and Fibronectin was utilized. ECMs were placed into solution at the above concentrations and allowed to coat tissue culture-treated polystyrene surfaces according to manufacturer's suggestions of 1 hour at room temp. Excess ECM solution was then removed and surfaces were rinsed twice in water. Just before seeding cells, the water was aspirated, then cells were seeded onto the ECM surface at a density of 1×10⁵ cells/cm² in growth medium (PCM) composed of DMEM:HamsF12 mixture (1:1) with 4 mM glutamine, 1×ITS supplement (GIBCO 51500-056), 10% Fetal Calf Serum (Inactivated, Qualified, GIBC 26140-079), and 10 ng/ml Epidermal Growth Factor (EGF) (BD 4001). Cells were seeded onto tissue-culture polystyrene surface as a control. After 18 hours, unattached cells were washed away and remaining attached cells were re-fed with PCM and allowed to grow for 7 days prior to evaluation. Cultures were fixed in 10% formalin and subjected to immunocytochemistry with antibodies for CK19 and Amylase as described previously to determine phenotypic composition. Cells were counterstained with DAPI fluorescent blue nuclear stain to visualize individual cell nuclei for cell counting. The metabolic activity of cells subjected to the various conditions was determined by an MTS assay. Viable cells were measured using the MTS assay (Promega CellTiter 96 Aqueous One Solution Cell Proliferation Assay), a colorimetric method for determining the number of viable cells in proliferation or cytotoxicity. The results of this analysis are shown in FIG. 7.

### Example 2

### Further Studies with ECM Surfaces and Various Media Components

Primary pancreatic cells, composed of >90% non-islet/non-duct cells, were plated onto various coated surfaces at a density of 28,900 cells/well (10⁵ cells/cm²). Unattached cells were washed off after 18 hours, and cultures were re-fed and allowed to grow for 8 days. Cultures were fixed in formalin (10%) and subjected to phenotypic analysis with antibodies to CK19 and Amylase. The results are shown in FIG. 4A-B. While Collagens I, IV, Laminin, Fibronectin, and Matrigel provide a suitable surface for cell attachment and expansion, maintenance of acinar (amylase+) phenotype along with the presence of an increased proportion of cells with a glandular epithelial phenotype (CK19+) was superior on Collagen I. More than 50% of cells analyzed expressed amylase and more than 50% of cells analyzed expressed CK19, suggesting that a subpopulation of cells in these experimental conditions express both markers.

Tissue culture-treated polystyrene culture surfaces were coated with Collagen I as described above. Tissue culture medium (PCM) was prepared as described above. In some cases, serum was replaced with Fraction V BSA (99% pure, heat inactivated, Sigma), along with combinations of soluble growth factors, including IGF1, IGF2, betacellulin, HGF, EGF, and TGF-alpha. Optimal seeding density is between 10⁴ and 10⁵ cells/cm², as demonstrated in Example 3. Cells were seeded onto collagen-coated flasks (150 cm²) at 1.5×10⁶ cells/flask in PCM. After an ˜18 hr attachment period, unattached cells were washed away with gentle aspiration/rinse, followed by re-feeding with fresh medium. Cultures were monitored over time by metabolic assay (MTT) and by trypsinization and cell counting, to establish cell number (see example 3). Cell phenotype at the end of the culture period was assessed as follows: small-scale cultures were set up simultaneously in 96-well plates. At the end of the culture phase, monolayer cells were fixed in 10% formalin for a minimum of 1 hour. After formalin was removed and monolayers were rinsed, cultures were subjected to immunocytochemistry as described in previous section for CK19, amylase, insulin, and vimentin (a marker of fibroblasts). The relative fraction of CK19+ cells was determined by quantitative image analysis as described above (see Example 4). After formalin was removed and monolayers were rinsed, cultures were subjected to immunocytochemistry as described in previous section for CK19 and vimentin (a marker of fibroblasts). Cells were also stained with amylase antibodies, but did not produce positive results due to release of digestive enzymes, such as amylase, by the cells over time in culture. The relative fraction of CK19+ cells was determined by quantitative image analysis as described above (see example 4). Acquisition of ductal markers by acinar cells was verified by demonstrating concomitant expression of CK19 and amylase in cell subpopulations during days 2-3 of culture (see example 5). For these experiments, CK19 primary antibodies were reacted with formalin-fixed cell cultures, followed by visualization with Alexa488-conjugated Goat anti-mouse IgG (Molecular Probes). Then, cells were subjected to a blocking step (Protein Blocker, BioGenex), followed by application of the second primary antibody (anti-amylase). Visualization of the amylase was accomplished by application of Alexa594-conjugated Goat Anti-Mouse IgG. Images were collected as described above. At the end of a 7-day culture period in the conditions described herein, between 65-90% of the cells in the culture express CK19, while less than 20% express vimentin (see example 6). Variations in the relative proportion of CK19+ cells probably reflect heterogeneity due to age, gender, and other unique characteristics of individual patients.

### Example 3

### Density of Cell Seeding

Primary pancreatic cells were seeded at (3) densities on tissue-culture treated polystyrene dishes (60 mm) and fed with PCM. Light microcopic observations were made daily. At the 24-hour timepoint, dishes were sacrificed and stained with trypan blue to assess viability. The results are shown in Table 3.

TABLE 3

| | | | After 3 days |
|---|---|---|---|
| Seeding Density | At 24 Hours: | At 48 Hours: | Growth: |
| 10⁴ cells/cm² | Most cells attached, | Mitotic Figures present | Epithelial |
| | trypan blue negative | (light microscopy) | monolayer |
| | (live) | | forming |
| 10⁵ cells/cm² | Most cells attached, | Mitotic Figures present | Epithelial |
| | trypan blue negative | (light microscopy) | monolayer |
| | (live) | | forming |
| 10⁶ cells/cm² | Some cells attached, | Few Mitotic Figures | Cells are |
| | most are trypan blue positive | present (light microscopy) | detached; some |
| | (dead) | | fibroblasts present |

### Example 4

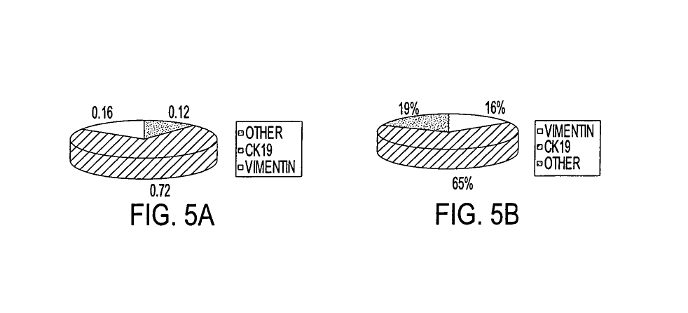

Cells were grown on a Collagen I surface, at 37° C. in 21% oxygen, in PCM medium or in base medium with 2% BSA, 2 ng/ml TGF-α, 10 ng/ml EGF, and 10 ng/ml HGF. After 7 days, cultures were fixed in 10% formalin and subjected to immunocytochemical analysis with fluorescent detection, followed by automated image collection and analysis. The results are shown in FIGS. 5A and 5B. Fibroblast (vimentin+) fraction, glandular epithelial cell fraction (CK19+), and fraction of unlabeled cells (Other) are similar after expansion. This suggests that replacement of serum with the serum-free medium maintains fraction of CK19+ cells without overgrowth of fibroblasts as compared to cells grown in serum-containing media.

### Example 5

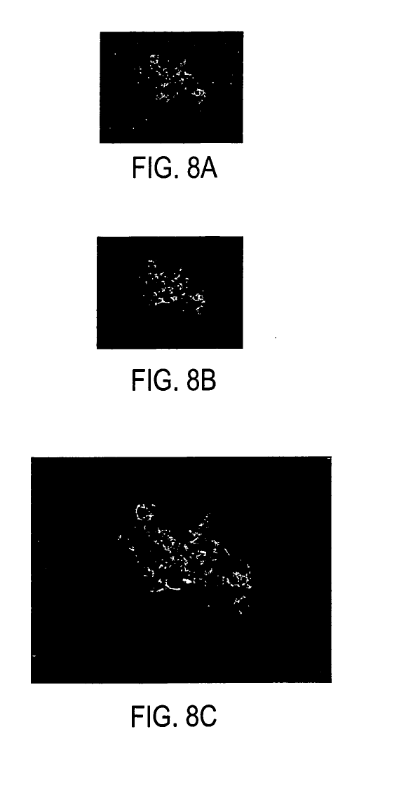

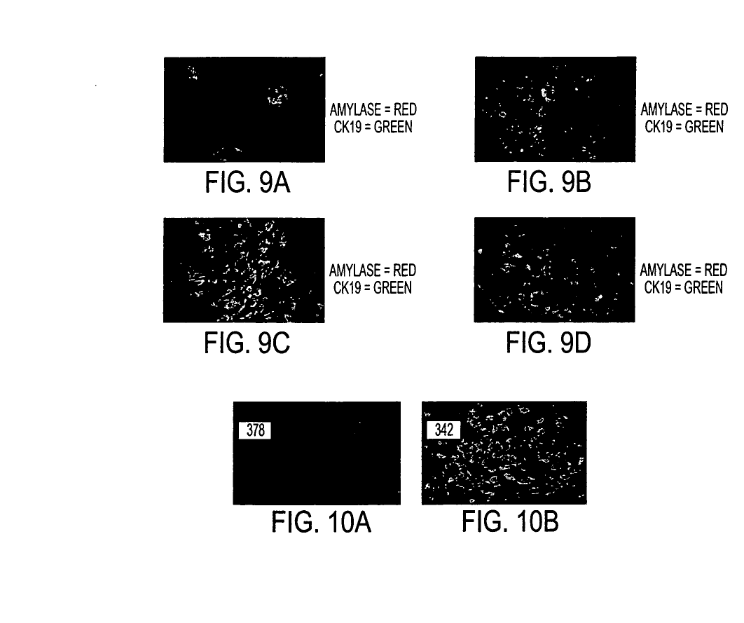

Primary pancreatic acinar cells were cultured for several days in a 1:1 ratio of DMEM and HamsF12, with 10% fetal bovine serum, 0.01 mg/ml insulin, 0.0055 mg/ml transferrin, 0.0067 μg/ml sodium selenite, 10 ng/ml EGF, 4 mmol/liter glutamine and antibiotics. After 2 days of culture (4 days ex vivo), expression of amylase by the acinar cells is still strong (FIG. 8A, upper left panel, red staining) as determined by immunocytochemistry. Expression of CK19 is also apparent (FIG. 8B, lower left panel, green staining). Overlay of the two images (FIG. 8C) demonstrates clear co-expression of amylase and CK19 in a large proportion of the cells, indicating that an intermediate cell exists from active conversion of amylase+ acinar cells to an amylase+/CK19+ mixed acinar/ductal phenotype (AD cells). Daily evaluation of cultures (FIG. 9) demonstrated that onset of CK19 expression begins around Day 2 of culture and by Day 5 cultures have lost most immunodetectable amylase expression and CK19 expression is prevalent.

### Example 6

After 7 days of growth in PCM/Collagen I surface, cells were fixed, stained with antibodies to CK19, and counterstained with nuclear DAPI. Total cell number was evaluated by automated image analysis (FIG. 10A left panel, blue-stained cell nuclei), while CK19+ cells were counted (FIG. 10B, right panel, green-stained cell cytoplasm). Of 378 total cells, 342 were immunopositive for CK19 (90%). After approximately 7 days of culture using conditions described herein, the acinar cells have concrete ductular characteristics, now referred to as IP cells. For most primary human cultures, more than 80% of cells in the culture after about 7 days express markers such as CK19 that are associated with ductular cells from a variety of tissues.

### Example 7

### Gene Expression Analysis of 7-Day Cultures (IP Cells)

Two independent IP cell cultures were subjected to Clontech 8K Atlas Gene Array analysis. IP cells were obtained by culturing primary acinar cells in a cell culture system comprising PCM and a Collagen I surface. Monolayer cultures were rinsed 2× with PBS, then detached from the flasks with 0.25% trypsin. Cells were pelleted by centrifugation at 1,200 RPM for 3 minutes in a swinging bucket centrifuge. Cell pellets were resuspended and washed 2× in PBS before a final centrifugation at 1,200 RPM for 3 minutes as described above. The supernatant was discarded and gently aspirated to remove as much liquid as possible from the cell pellet, which was then quick-frozen in a dry-ice/ethanol bath and stored at −80° C. until transfer to BD Clontech where gene expression analysis was performed, using conventional techniques.

Labelled P-33 cDNA probes were prepared from the 30 μg of total RNA from each sample by first enriching for poly A+RNA using a streptavidin-magnetic bead separation method that is part of the Atlas Pure Total RNA Labeling system. The labeled probes from each sample were hybridized with the plastic human 8 K gene arrays for about 16 hours, the arrays were washed and imaged according to the Atlas array protocols. The Atlas image 2.7 software was used to align array images with the array grid template and to exclude false background signals or false signals due to strong signal bleedover. The transcript signals were then extracted from these aligned arrays using the Atlas Image 2.7 software and further statistical analysis of the changes in gene expression were performed.

In general, mRNA transcription was assayed, by hybridization to suitable oligonucleotide probes. In a few cases, e.g., for CK19 and amylase, the protein expression product was measured, using conventional methods of immunohistochemistry. A summary of the expression by these cell populations of a selected set of genes is presented in Table 4. Table 4 contains a list of genes expressed in IP cells and a comparison of expression patterns in primary acinar cells and primary ductal cells. Gene products identified as “+” were expressed; those identified as “++” were strongly expressed. Gene products designated) are found in regenerating pancreas.

TABLE 4

| | | | Primary |
|---|---|---|---|
| | | | Acinar |
| Gene | IP Cells | Primary Ductal Cells | Cells |
| Aquaporin 1 | + | ++(mRNA)/+(protein) | 0 |
| Aquaporin 5 | ++ | +(mRNA)/−(protein) | |
| Aquaporin 8 | + | − | + |
| Insulin Receptor Substrate-2 | + | ++ ® | |
| Protein Kinase B (AKT) | 0 | + | + |
| Calpain (mu) | ++ | − | ++ |
| CFTR (Cystic Fibrosis | 0 | 0 | 0 |
| Transmembrane | | | |
| Conductance Regulator) | | | |
| Claudin 2 | nd | ++ | − |
| Claudin 3 | + | + | + |
| Claudin 4 | + | + | + |
| Claudin 5 | nd | − | ++ |
| Carbonic Anhydrase II | 0 | 0 | 0 |
| Inositol 1,4,5 triphosphate | ++ | − | ++ |
| Rc, Type 3 | | | |
| MUC-1 | + | ++ | + |
| MUC-6 | ++ | + | |
| Cytokeratin 7 | ++ | ++ | − |
| Cytokeratin 8 | ++ | + | |
| Cytokeratin 18 | + | + | |
| Cytokeratin 19 | + | ++ | − |
| alpha v integrin | 0 | + | |
| alpha 3 integrin | ++ | n/a | |
| beta 3 integrin | 0 | + | |
| beta 4 integrin | + | n/a | |
| beta 5 integrin | 0 | + | |
| fibronectin | 0 | + | |
| collagen IV | + | + | |
| vitronectin | 0 | + ® | |
| MMP2 | 0 | Trace | |
| MMP9 | + | Trace | |
| TIMP1 | ++ | Trace | |
| TGF-alpha | Trace | ++ ® | |
| Gastrin | 0 | ++ ® | |
| ICAM-1 | 0 | 0 | |
| ICAM-2 | 0 | 0 | |
| ICAM-3 | 0 | 0 | |
| Pancreatitis Associated | ++ | + ® | Trace |
| Protein (PAP) | | | |
| Reg-1 | + | 0 | Trace |
| pyrimidinergic receptor P2Y | + | + | |
| Syndecan | + | +(low) | Trace |
| Glutathione S Transferase - | + | + | − |
| pi | | | |

II. Transformation of Glandular Epithelial Cells into Insulin Producing Cells—Generating Insulin-Producing Cells by Differentiation of IP Cultures (Culture Phase II)

IP cultures can be utilized to generate insulin-producing cells by placing the cells in a second phase of culture that includes a surface, such as Collagen I, that promotes attachment of the IP cells combined with a defined medium formula that lacks serum but contains combinations of the following differentiation promoting factors: Activin A, acidic FGF, basic FGF, C-Natriuretic Peptide (CNP), Calcitonin Gene Related Peptide, Cholera Toxin B Subunit, Dexamethasone, Gastrin-Releasing Peptide, Glucagon-like Peptide-1 (GLP-1), Glucose, IGF1, IGF2, Insulin, Laminin, LIF, Met-Enkephalin, PDGFAA+PDGFBB, Prolactin, Sonic Hedgehog, Substance P, TGF-alpha, Trolox (alpha-tocopherol derivative), and VEGF. In the following examples, the base medium is composed of a 1:1 mixture of HamsF12 and DMEM with antibiotics and 0.2% Bovine Serum Albumin (Fraction V, heat inactivated 99% pure). In one example (Combination 1), the base medium contained Cholera Toxin B, Dexamethasone, GRP, GLP-1, Glucose, IGF-1, IGF-2, Insulin, Prolactin, Sonic Hedgehog, Trolox, aFGF, and bFGF. In another example (Combination 2), the base medium contained Activin A, CGRP-alpha, CNP, Glucose, GLP-1, IGF-2, Insulin, LIF, Met-Enkephalin, Prolactin, Sonic Hedgehog, aFGF, and vEGF. In a third example (Combination 3), the base medium contains Activin A, CGRP-alpha, Cholera Toxin B, Dexamethasone, Glucose, GLP-1, Insulin, LIF, Laminin, Met-Enkephalin, PDGFAA/BB, Sonic Hedgehog, Substance P, TGF-alpha, aFGF, and VEGF. The concentrations of these media supplements are listed in Table 1.

AD cells were placed into culture by either: 1) trypsinizing the cells from the surface on which they were generated, and redistribution onto a fresh attachment-promoting surface at a density of ˜5×10⁴ cells/cm², or 2) removing the medium, washing 2× in PBS to remove traces of old medium, and cultures re-fed with the new medium (described above) containing differentiation promoting factors. Cells are cultured for a period of 4-10 days at 37° C. and 21% oxygen. On Day 5, half of the medium is removed and replaced with an equal volume of fresh medium containing differentiation promoting factors.

Phenotypic Analysis of IP cells After Differentiation Culture.

Morphological assessment of IP cells cultured in differentiation conditions described above was captured by light microscopy (see Example 8, below). The cellular phenotype of the cells comprising these cultures was assessed by immunocytochemistry as described above using monoclonal antibodies to vimentin, pro-insulin, C-peptide, MUC-1, and CK19 (See Example 10, below). Briefly, cultures were fixed with 10% formalin for 1 hour at room temperature, then washed with PBS and subjected to immunocytochemical protocol. (See Example 9, below).

Functional Analysis of IP cells After Differentiation Culture.

The ability of the aggregated cell clusters to release insulin and C-peptide was assessed by subjecting the cultured cells to a glucose challenge as follows. Cells that had been cultured in differentiation medium for 7-10 days were washed 3× in PBS, then re-fed with either 1) base medium (described above) with 5 mM Glucose, or 2) base medium with 22 mM glucose. After 18 hours, the cell-conditioned medium was collected and subjected to ELISA analysis for insulin and C-peptide release (Diagnostic Systems Laboratories (DSL)). ELISAs were conducted using the standard range assay procedure according to manufacturer's specifications. Plates were incubated on a shaker during the assay and results were read in a Tecan spectrophotometric plate reader. Total ng of insulin or C-peptide per well were calculated for each media condition, for both 5 mM glucose media and 22 mM glucose media (See Example 10).

### Example 8

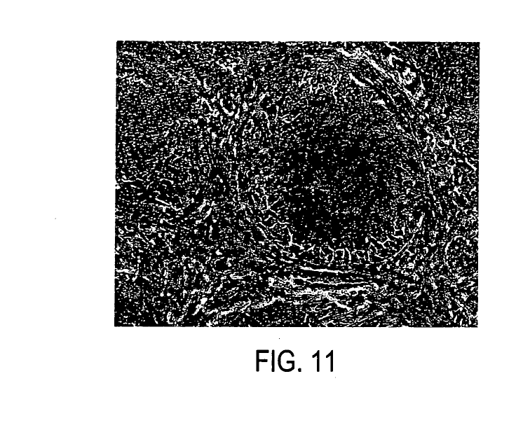

Pancreatic acinar cells were cultured in Base Medium+ITS+Serum (10%) for 1 week, then trypsinized (treated with 0.25% Trypsin without EDTA for 10 minutes at 37° C.) and transferred to a fresh collagen-1 coated surface and placed in a medium containing all 23 DFPs listed. Over a period of 3-5 days, the cells readily formed three-dimensional pod-like structures, clearly observable by light microscopy (FIG. 11). Some larger pods detached from the culture surface after about 4-6 days in culture, and remained viable, as determined by trypan blue exclusion. The pod-like structures were hypothesized to be aggregations of insulin-producing cells, and subjected to further analysis as described below.

### Example 9

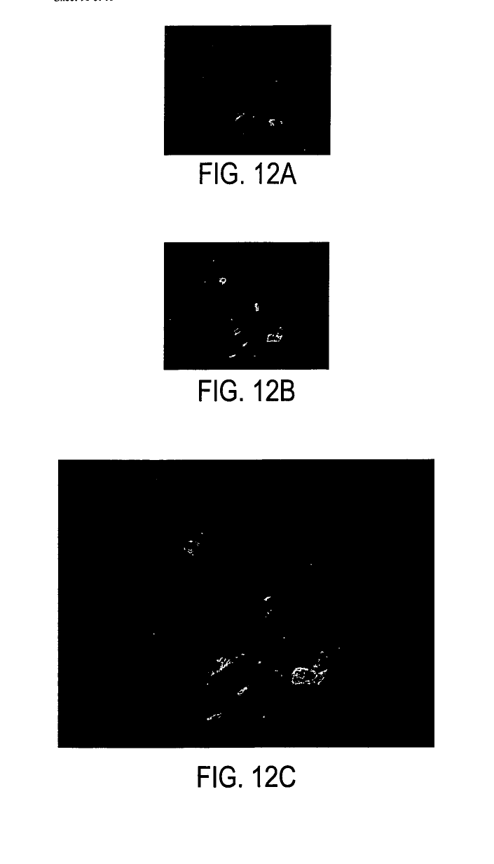

Pod-like structures, generated the same manner as described in the previous example, were fixed in 10% formalin and subjected to immunocytochemical analysis first with CK19 monoclonal antibodies, then with C-peptide monoclonal antibodies, as described above. FIG. 12A shows a group of cells (DAPI stained nuclei are blue), some of which are immunopositive for CK19 (green staining). FIG. 12B shows the same group of cells, many of which are positive for C-Peptide, which is produced when the proinsulin molecule synthesized within the cell is cleaved to yield mature insulin; the C-peptide stained cells are red, with a typical granular staining of the cytoplasm. FIG. 12C shows a higher power overlay image, demonstrating colocalization of CK19 and C-peptide in a small subset of cells. Co-stained cells appear yellow-orange on the overlay image.

### Example 10

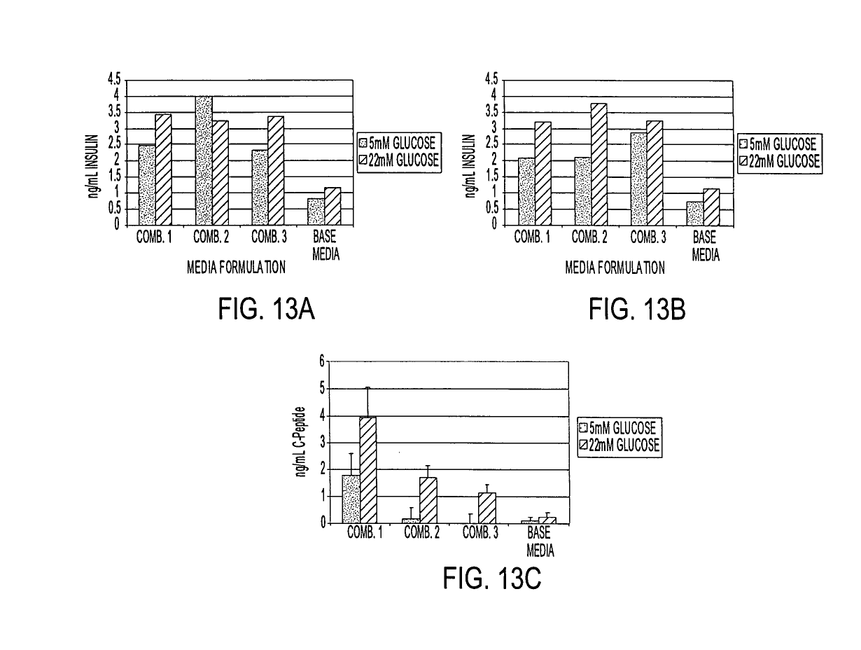

Cells cultured in base medium (negative control), or in Combinations 1, 2 and 3 of the differentiation promoting media, were evaluated for their ability to release insulin and C-peptide into the culture medium. In addition, we assessed whether increasing concentrations of glucose led to the release of a greater quantity of insulin and C-peptide, indicating an islet-like functionality. First, the cells were cultured for 1 week in base medium+EGF (10 ng/ml)+ITS+10% fetal bovine serum (PCM). Then, cells were either subjected to a wash and medium change (non-subcultured), or to a wash, trypsinization/detachment, reseeding, and medium change. Replicate cultures were re-fed with either base medium (serum-free), fresh PCM, or one of the three combinations of differentiation promoting media (all serum-free). After 10 days, differentiation media were removed, cultures were washed 3× with PBS, then re-fed with serum-free base medium containing either 5 mM glucose or 22 mM glucose (final concentration). After 18 hours, the conditioned media were collected and subjected to ELISA analysis with antibodies to either Insulin or C-Peptide (DSL laboratories). FIGS. 13A, 13B and 13C, respectively, show insulin release by nonsubcultured cells, and insulin release and C-peptide release in response to glucose challenge. Since some of the cultures contain insulin, and cells can take insulin up from the medium, production of C-peptide is an important confirmation that the cells are synthesizing insulin de novo from the synthesis and processing of proinsulin. Furthermore, the production of insulin and C-peptide is increased in most cases with increasing glucose concentration, suggesting an islet-like function of cells within these cultures. Note that little insulin or C-peptide is produced in the base medium that contains no DPFs.

### Example 11

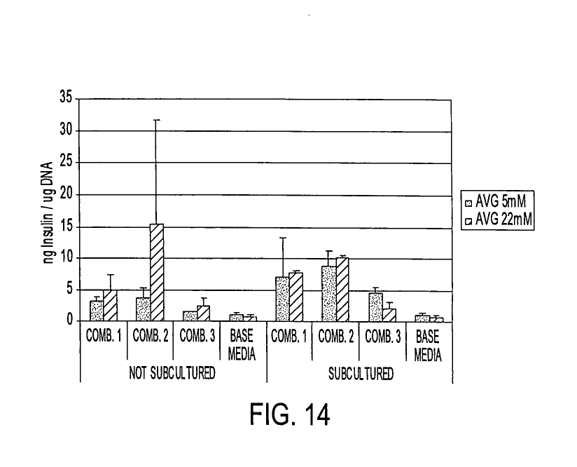

Both the quantity of insulin and the quantity of DNA were measured in IP cells subjected to differentiation culture with or without enzymatic detachment and subculturing. Cultures were carried out precisely as described in the previous paragraph. DNA was measured utilizing a standard Picogreen assay (Molecular Probes), while insulin was measured by ELISA assay. Total ng of Insulin was divided by total μg of DNA in the sample, thus providing the insulin:DNA ratio value, in order to calculate a ratio of the quantity of insulin present vs. the number of cells present (reflected by DNA content). The results are shown in FIG. 14. In each of the differentiation media combinations, the insulin:DNA ratio is increased compared to base medium, suggesting that more insulin is produced on a per cell basis in the presence of DPFs than when cultured without them. Furthermore, the insulin:DNA ratio is increased slightly in some conditions upon glucose challenge (22 mM glucose vs. 5 mM), suggesting that the cells respond to glucose by releasing a greater quantity of insulin.

### Example 12

Insulin-producing cells obtained by the preceding method were subjected to gene expression analysis as described above. Table 5 contains a list of the highest expressed genes, their position on the Clontech atlas 8K gene array, and relative expression of these genes (after normalization). Table 5 is attached hereto as Appendix 1

### Example 13

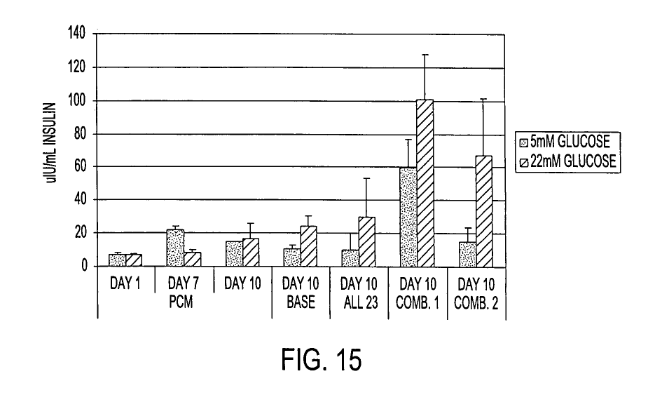

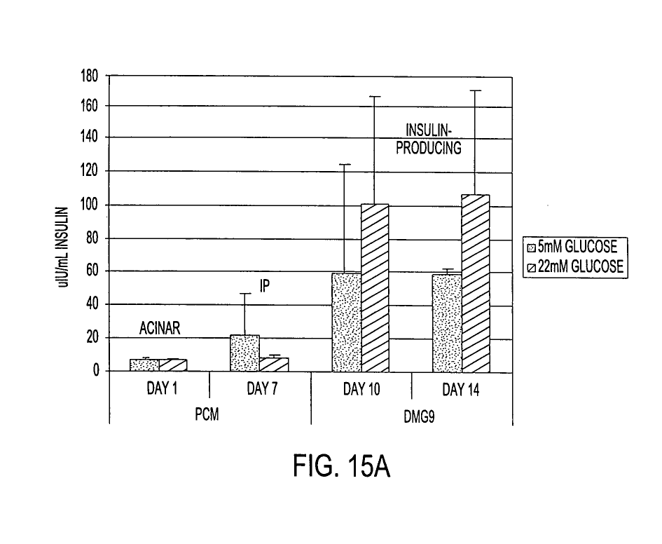

Primary human pancreatic cells were seeded at 0.5×10⁵ cells/cm² in PCM on a collagen-1 surface and grown for 7 days. Insulin was measured at Days 1, 7, and 10 as follows: Growth medium was removed, wells were washed 3× in phosphate buffered saline. After a pre-incubation for 1 hour at 37 C in base medium without insulin, with 5 mM glucose, media was removed and replaced with either 1) base medium (without insulin) with 5 mM glucose, or 2) base medium (without insulin) with 22 mM glucose. Insulin was measured in cell-conditioned media after 18 hours at 37° C. After 7 days of culture, PCM medium was replaced with either 1) fresh PCM, 2) serum-free base medium, 3) serum free base medium with all 23 differentiating factors, 4) serum-free combination 1, or 5) serum-free combination 2. The results are shown in FIG. 15. After 3 days exposure to the differentiating factors, increased insulin release is noted in presence of differentiating factors. The results on Day 1 argue against the presence of a significant number of insulin-producing cells in the starting material, demonstrating the de novo generation of insulin-producing cells from acinar cells in the primary culture. It can be seen in the Figure that at the end of 10 days, insulin release in response to a glucose challenge is much greater in the DFP media than in the PCM or base medium, verifying the stimulatory effect that the DFPs exert on transformation of glandular epithelial cells into insulin-producing cells.

### Example 14

Human pancreatic acinar cells were cultured on a collagen I surface in PCM from Day 1 to Day 7, thus generating a culture of IP cells at Day 7. On Day 7, the EP cells were washed and the PCM medium was replaced with the G09 differentiation medium containing the 30 factors listed in Table 2. At each time point (Days 1, 7, 10 and 14), insulin release was measured by washing the cultures three times with PBS, then challenging the cultures with a 1:1 mixture of DMEM and HAMs F12 containing either 5 mM or 22 mM glucose. After 18 hours of exposure to the glucose, supernatants were collected and insulin measured by ELISA. The results are shown in FIG. 15a.

III. Expression Studies at Several Time Points of Primary Human Acinar Cells that are Expanded, Allowed to Differentiate into IP Cells and then Allowed to Differentiate Further into Insulin-Producing Cells

### Example 15

Three independent samples of primary human pancreatic acinar cells were seeded and expanded described above. From Day 0 to Day 8, cells were on collagen I surface, seeded at 10⁴ cells/cm², in PCM. On Day 8, the medium was changed from PCM to the medium with the active factors shown in Table 2. Cells were fed twice with G09 (50% of medium replaced) between days 8 and 16. The cells remained on the surface throughout the culture process. Cultures were harvested at 3 days after the initial plating (actively trans-differentiating acinar cells), 8 days after plating (IP cells) and 16 days after plating (putative insulin producing cells) and subjected to gene expression analysis, as described in Example 7. mRNA expression data were obtained with 12K microarrays from Clonetech.

Briefly, growth medium was removed from the culture flasks and cells were lysed in trizol LS (Invitrogen) chaotrope/phenol reagent for about 2 minutes by pipetting the lysis solution over the cell layer. Three ml of RNAse free water was added per 9 ml of lysis solution in an Oak Ridge Cetrifuge tube. 2.4 ml chloroform was then added and the solution vigorously vortexed for 1 minute. The aqueous and organic phases were then separated by cetrifugation at 4° C. and the upper aqueous phase containing RNA was removed to a clean PET tube. The RNA was precipitated by isopropanol precipitation, washed with 70% ethanol and redissolved in 200 μl of RNAse free water. A chaotrope lysis reagent was immediately added to the RNA and it was further purified using a Qiagen spin column method with a DNAse digestion step. The purified RNA was finally eluted in 80 μl RNAse free water and stored at −80° C.

Labelled P-33 cDNA probes were prepared from the 30 μg of total RNA from each sample by first enriching for poly A+RNA using a streptavidin-magnetic bead separation method that is part of the Atlas Pure Total RNA Labeeling system. The labeled probes from each sample were hybridized with the plastic human 12 K gene arrays for about 16 hours, the arrays were washed and imaged according to the Atlas array protocols. The Atlas image 2.7 software was used to alighn array images with the array grid template and to exclude false background signals or false signals due to strong signal bleedover. The transcript signals were then extracted from these aligned arrays using the Atlas Image 2.7 software and further statistical analysis of the changes in gene expression were performed.

The raw expression data were analyzed as follows: (1) We filtered out genes that were not expressed at any of the 3 conditions/time points; (2) We normalized all of the microarrays against each other to remove differences from array-to-array and the effects of variability in sample processing, hybridization, etc.; (3) We identified genes which showed a statistically significant difference among the conditions/time points; and (4) We clustered the genes based on their temporal patterns in a way that is consistent with the design of the study and the changes in phenotype.

Table 6 shows expression data for the genes that were identified by the above analysis. This Table is attached hereto as Appendix 2. These identified genes were expressed at high levels at both Day 3 and Day 8, or their expression increased substantially between Day 3 and Day 8. The Table also shows the expression levels of these genes at Day 16, and the mean expression for all three condition/time points. The Table also shows the ratios of expression at various times: “I to A” is the ratio of expression of putative insulin-producing cells (Day 16) to acinar (Day 8) cells; “Int to A” is the ratio of IP cells (Day 8) cells to acinar cells (Day 3).

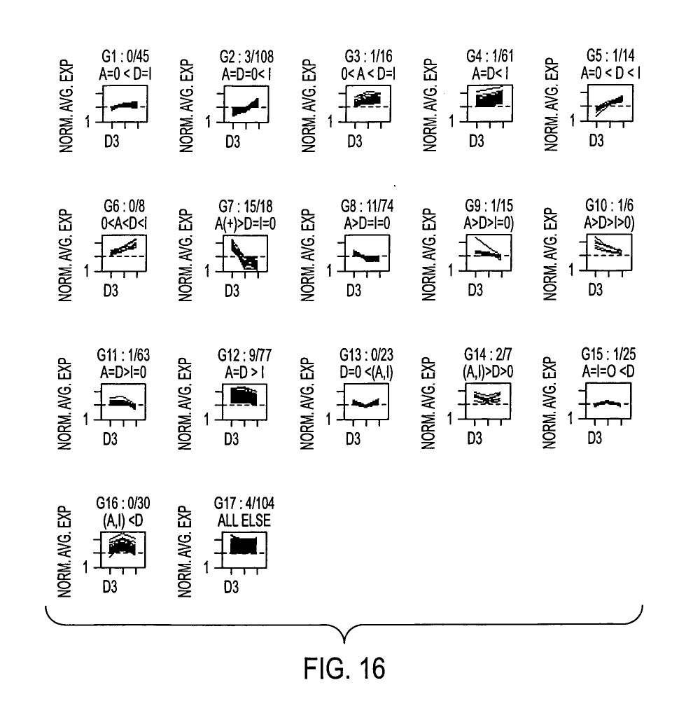

The data shown in Table 6 were further analyzed by clustering them into one of 17 “classes,” whose features are summarized on the Table. A graphical representation of the characteristics of these 17 classes in presented in FIG. 16.

The data from the Day 8 time points in Table 6 were also grouped with regard to whether the genes expressed at Day 8 in these cells belong to the classes of genes expressed normally in (1) liver and pancreas; (2) pancreas-associated genes; (3) liver-associated genes; or (4) progenitor-associated genes. The results are shown in Table 7.

TABLE 7
BDT Intermediate Cells

| Genes Expressed in Liver and | | | | | Pancreatic Pattern of |
|---|---|---|---|---|---|
| Pancreas | Sample 1 | Sample 2 | Sample 3 | Hepatic Pattern of Expression | Expression |
| CK18 | + | + | + | hepatic lineage | acinar cells |
| CK8 | + | + | + | hepatic lineage | acinar cells |
| CK19 | + | + | + | bile duct | duct cells |
| CK7 | + | + | + | bile duct | duct cells |
| HNF1 | + | + | + | liver tc factor | in beta cells |
| α-1 antitrypsin | ++ | ++ | ++ | produced in differentiated hepatocytes | yes |
| Notch-1 | − | − | Trace | involved in differentiation of cells into | developing pancreatic |
| | | | | hepatic lineage | epithelium |
| α-fetoprotein | − | − | − | developing hepatocytes and hepatic | developing pancreatic |
| | | | | progenitors | ducts |
| Notch-3 | Trace | Trace | Trace | involved in differentiation of cells into | pancreatic mesenchyme |
| | | | | hepatic lineage | and endothelium |
| Notch-4 | Trace | Trace | Trace- | involved in differentiation of cells into | pancreatic mesenchyme |
| | | | | hepatic lineage | and endothelium |
| Jagged-2 | + | + | + | involved in differentiation of cells into | yes |
| | | | | hepatic lineage | |
| pi-glutathione s | + | + | ++ | developing liver | duct cells and centroacinar |
| transferase (pi-GST) | | | | | cells |
| γ-glutamyl transferase | − | − | − | developing bile duct | acinar cells |

| | | | | | Pancreatic Pattern of |
|---|---|---|---|---|---|
| Pancreas-Associated Genes | Sample 1 | Sample 2 | Sample 3 | Hepatic Pattern of Expression | Expression |
| carbonic anhydrase | − | − | Trace | no | duct cells |
| CF transmemb conductance regulator | − | − | Trace | no | duct cells |
| elastase | − | − | − | no | acinar cells |
| amylase | − | − | − | no | acinar cells |
| insulin | + | Trace | Trace | no | islet |
| somatostatin | Trace | Trace | Trace | no | islet |
| Pancreatic Polypeptide | + | + | Trace | no | islet |
| Glucagon | − | − | − | no | islet |

| Liver-Associated | | | | | Pancreatic Pattern of |
|---|---|---|---|---|---|
| Genes | Sample 1 | Sample 2 | Sample 3 | Hepatic Pattern of Expression | Expression |
| Sialyltransferase-6 | − | − | − | produced in differentiated hepatocytes | no |
| Liver-specific bHLH | + | + | + | liver-specific Tc factor | no |
| transcrip factor | | | | | |
| Thy-1 | + | + | + | hepatic oval cell marker | no |
| Glucose-6-phosphatase | − | − | Trace | hepatic lineage, progenitors and adult | No |
| Glutamine synthetase | − | − | Trace | hepatocyte | No |
| Carbamoyl phosphate | − | − | − | hepatocyte | no |
| synthetase-1 | | | | | |
| Dipeptidylpeptidase IV | − | − | − | hepatocyte | no |
| C/EBP-α | ++ | ++ | + | liver-specific Tc factor | *turned on and upregul |
| | | | | | during hepatization of |
| | | | | | pancreas (copper-deficient |
| | | | | | diet) |
| C/EBP-beta | ++ | ++ | ++ | liver-specific Tc factor | *turned on and upregul |
| | | | | | during hepatization of |
| | | | | | pancreas (copper-deficient |
| | | | | | diet) |

| Progenitor Cell-Associated Genes | Sample 1 | Sample 2 | Sample 3 | Tissue |
|---|---|---|---|---|
| Musashi-1 | − | − | − | Intestine |
| Nestin | − | − | − | Pancreas/Neuronal |
| CD34 | − | − | − | Hematopoietic |
| Thy-1 | − | − | − | Hepatic progenitors |
| BMP-2 | − | − | − | Neuronal |
| BMPRcIA | − | − | Trace | Mesenchyme |
| c-kit | − | − | − | Liver/Pancreas/Neuronal |
| chromogranin A | Trace | Trace | Trace | Neuroendocrine/Liver/Intestine |
| PDX-1 | − | − | + | Pancreas |

As can be seen, at Day 8 IP cells no longer expressed genes consistent with pancreatic acinar cells, nor did they express a complement of genes specific for pancreatic ductular cells. The IP cells expressed low levels of some markers associated with pancreatic islets, including insulin, somatostatin and pancreatic polypeptide, suggesting that at least some cells in the population are competent to express endocrine genes of the pancreatic islets.

Surprisingly, the IP cells also expressed several liver-specific transcription factors (e.g., C/EBP alpha, C-EBP-beta) and other markers of mature and developing liver, including low levels of Thy-1, a marker associated with hepatic “oval” stem cells. This suggests that the differentiating cells were not moving simply from pancreatic acinar to pancreatic ductal, but had developed into a cell with both hepatic and pancreatic characteristics, while not fitting into any single gene expression profile of one of these cell types. The cells generated in this example resemble the cells that emerge from the pancreas of rodents that are fed a copper-deficient diet. (See, e.g. Rao et al., 1988). The pancreas of such animals goes through an acute phase of pancreatitis followed by “hepatization” of liver (which means cells that begin to express hepatic genes rather than pancreatic genes). Liver-like cells have also been reported in human fetal pancreas (Tsanadis et al., 1995) Isolated cells generated by the methods of the present invention (e.g., by propagating primary acinar cells or other types of endodermal cells or progenitor cells by the methods of the invention) are to be distinguished from naturally occurring cells that may have some of the characteristics of IP cells, such as oval cells or cells isolated from the pancreas of a rodent on a copper-deficient diet.

Cells having the characteristics of these IP cells may be useful for, e.g., therapeutic approaches in the treatment of diabetes. Furthermore, although the cells in this example were derived from pancreas, other epithelial tissues, or perhaps even any endoderm-derived tissue, may provide additional sources of cells that can be differentiated into cells having a similar phenotype. Suitable tissue types include, e.g., liver or intestine. These IP cells express genes associated with pancreas, liver, intestine and neuronal tissues. For example, they express mucin, CK19 and CK7, which are common markers associated with duct cells in the pancreas, liver and intestine. Thus, the gene expression pattern seen in these IP cells may serve as a predictive measure for cells derived from each of these tissues for the purpose of generating insulin-producing cells. Furthermore, IP cells may, under appropriate conditions, give rise, not only to pancreatic islet cells, but also to hepatocytes or any endoderm-derived tissue.

The disclosures of the following references, cited above in part, relate to the present invention:
WO 02/29010 A2 (Kerr-Conte);
Bonner-Weir, S. et al., Proc. Natl. Acad. Sci. USA 97: 7999-8004 (2000),
Bouwens, L., Microsc. Res. Tech. 43: 332-6 (1998),
Bowens, L. et al. Diabetologia 41:629-33 (1998);
Gmyr, V. et al., Diabetes 49:1671-80 (2000);
Gmyr, V. et al. Cell Transplant 10:109-21 (2001),
Gmyr, V. et al. Diabetes 49:1671-80 (2000),
Hall, P. A. et al., J. Pathol. 166: 97-103 (1992);
Kerr-Conte, J. et al., Diabetes 45:1108-14 (1996);
Kerr-Conte, J. et al., Transplant Proc 27:3268 (1985);
Pattou F. et al., Bull. Acad. Natl. Med. 184:1887-99 (2000);
Rao, M S et al Biochem Biophys Res Comm. 156:131-6 (1988);
Rooman, Ilse et al., Diabetes 51: 686-90 (2002);
Rooman, I, et al. Diabetologia 43:907-14 (2000);
Rooman, I. et al., Gastroenterology 121: 940-9 (2001);
Trivedi, N. et al. Endocrinology 142:2115-22 (2001);
Tsanadis, G. et al. Histol. Histopathol. 10:1-10 (1995);
Wang, R. N. et al., Diabetologia 38:1405-11(1995);
U.S. Pat. No. 6,011,647 (Ammon Peck).

The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

The entire disclosure of all applications, patents and publications, cited above and in the figures are hereby incorporated in their entirety by reference.

### Appendix 1

TABLE 5

| GENE | Position on Atlas 8K Array | Relative Expression, Sample 1 | Relative Expression,Sample 2 |
|---|---|---|---|
| aquaporin 5 | 7547 | 4539 | 4550 |
| actin, beta | 3952 | 3598 | 3442 |
| actin, beta | 8176 | 3047 | 3747 |
| growth hormone secretagogue receptor | 6846 | 2092 | 1710 |
| profilin 1 | 448 | 1580 | 1246 |
| special AT-rich sequence binding protein 1 | 3035 | 1437 | 886 |
| (binds to nuclear matrix/scaffold- | | | |
| associating DNA's) | | | |
| cardiac-specific homeo box | 2050 | 1395 | 2850 |
| CCAAT/enhancer binding protein (C/EBP), | 1588 | 1374 | 1671 |
| beta | | | |
| RAS guanyl releasing protein 2 (calcium | 8262 | 1263 | 1616 |
| and DAG-regulated) | | | |
| cartilage paired-class homeoprotein 1 | 6677 | 1166 | 1241 |
| paired-like homeodomain transcription | 6805 | 1113 | 756 |
| factor 1 | | | |
| transcription factor 21 | 7621 | 1063 | 801 |
| CD3E antigen, epsilon polypeptide (TiT3 | 8054 | 994 | 1113 |
| complex) | | | |
| CD151 antigen | 5613 | 935 | 808 |
| ATPase, Ca++ transporting, plasma | 7940 | 919 | 876 |
| membrane 2 | | | |
| ficolin (collagen/fibrinogen domain- | 824 | 901 | 829 |
| containing) 3 (Hakata antigen) | | | |
| inositol polyphosphate phosphatase-like 1 | 5989 | 832 | 425 |
| protein tyrosine phosphatase, receptor | 8367 | 806 | 810 |
| type, S | | | |
| integrin, alpha 3 (antigen CD49C, alpha 3 | 1743 | 758 | 647 |
| subunit of VLA-3 receptor) | | | |
| syntaxin 1A (brain) | 6851 | 743 | 658 |
| parathymosin | 7135 | 725 | 852 |
| thymosin, beta 10 | 7436 | 709 | 1617 |
| midkine (neurite growth-promoting factor 2) | 2470 | 682 | 802 |
| arylsulfatase A | 3147 | 678 | 3084 |
| calbindin 2, (29 kD, calretinin) | 7000 | 676 | 786 |
| serine (or cysteine) proteinase inhibitor, | 5214 | 668 | 678 |
| clade A (alpha-1 antiproteinase, | | | |
| antitrypsin), member 1 | | | |
| retinal G protein coupled receptor | 7972 | 653 | 696 |
| myosin regulatory light chain 2, smooth | 2499 | 638 | 561 |
| muscle isoform | | | |
| butyrate response factor 1 (EGF-response | 7325 | 630 | 646 |
| factor 1) | | | |
| type I transmembrane receptor (seizure- | 7583 | 589 | 394 |
| related protein) | | | |
| type I transmembrane receptor (seizure- | 7583 | 589 | 394 |
| related protein) | | | |
| procollagen C-endopeptidase enhancer | 3593 | 576 | 713 |
| mitogen-activating protein kinase kinase | 7069 | 558 | 571 |
| kinase kinase 2 | | | |
| protease, serine, 1 (trypsin 1) | 6323 | 558 | 656 |
| pancreatitis-associated protein | 4576 | 536 | 2177 |
| angiotensin receptor 1B | 4093 | 516 | 491 |
| stratifin | 6489 | 503 | 581 |
| keratin 17 | 7903 | 500 | 364 |
| somatostatin receptor 3 | 6872 | 432 | 433 |
| myosin-binding protein H | 4692 | 428 | 96 |
| ephrin-A5 | 4202 | 411 | 381 |
| RAP1, GTPase activating protein 1 | 1965 | 407 | 263 |
| chymotrypsinogen B1 | 6604 | 405 | 830 |
| ankyrin-like with transmembrane domains 1 | 3577 | 395 | 431 |
| Misshapen/NIK-related kinase | 2439 | 391 | 341 |
| bone morphogenetic protein 6 | 4492 | 381 | 308 |
| splicing factor proline/glutamine rich | 713 | 375 | 662 |
| (polypyrimidine tract-binding protein- | | | |
| associated) | | | |
| claudin 12 | 2720 | 370 | 378 |
| matrix metalloproteinase 23A | 7491 | 368 | 138 |
| neurogranin (protein kinase C substrate, | 4465 | 366 | 335 |
| RC3) | | | |
| dopamine receptor D2 | 1649 | 362 | 234 |
| cardiotrophin 1 | 5283 | 359 | 507 |
| CCAAT/enhancer binding protein (C/EBP), | 7237 | 359 | 645 |
| alpha | | | |
| paired box gene 9 | 5206 | 335 | 201 |
| protein tyrosine phosphatase, receptor | 8235 | 331 | 250 |
| type, N | | | |
| keratin 8 | 7215 | 327 | 449 |
| claudin 7 | 280 | 325 | 173 |
| trophinin associated protein (tastin) | 462 | 323 | 360 |
| neuronal thread protein | 8356 | 322 | 366 |
| basic helix-loop-helix domain containing, | 6734 | 318 | 215 |
| class B, 2 | | | |
| annexin A2 | 4467 | 290 | 226 |
| cathepsin D (lysosomal aspartyl protease) | 7370 | 289 | 600 |
| Bicaudal D (Drosophila) homolog 1 | 6822 | 284 | 217 |
| lectin, galactoside-binding, soluble, 1 | 7331 | 280 | 310 |
| (galectin 1) | | | |
| keratin 7 | 7171 | 278 | 481 |
| glyceraldehyde-3-phosphate | 3953 | 278 | 307 |
| dehydrogenase | | | |
| aquaporin 6, kidney specific | 7591 | 277 | 258 |
| TNF receptor-associated factor 1 | 6037 | 269 | 270 |
| Rho GDP dissociation inhibitor (GDI) alpha | 5565 | 267 | 321 |
| calcium channel, voltage-dependent, | 3142 | 260 | 207 |
| gamma subunit 4 | | | |
| glutamate receptor, ionotropic, kainate 1 | 4776 | 244 | 296 |
| CCR4-NOT transcription complex, subunit 4 | 983 | 244 | 428 |
| filamin A, alpha (actin-binding protein-280) | 3457 | 241 | 233 |
| peanut (Drosophila)-like 1 | 711 | 233 | 310 |
| enhancer of rudimentary (Drosophila) | 5921 | 231 | 405 |
| homolog | | | |
| endothelin converting enzyme 1 | 7835 | 229 | 197 |
| protocadherin 17 | 2483 | 223 | 208 |
| aquaporin 8 | 7679 | 217 | 145 |
| synapsin I | 4871 | 216 | 164 |
| Tubulin, alpha, brain-specific | 2827 | 212 | 345 |
| CD44 antigen (homing function and Indian | 7848 | 207 | 141 |
| blood group system) | | | |
| cerebral cavernous malformations 1 | 2731 | 205 | 116 |
| insulin-like 3 (Leydig cell) | 666 | 202 | 365 |
| adenylate cyclase activating polypeptide 1 | 4489 | 200 | 74 |
| (pituitary) | | | |
| hairless (mouse) homolog | 5185 | 196 | 36 |
| insulin | 4427 | 190 | 44 |
| granulin | 928 | 184 | 201 |
| granulin | 928 | 184 | 201 |
| early growth response 1 | 2111 | 178 | 136 |
| cyclin-dependent kinase inhibitor 1A (p21, | 7852 | 175 | 108 |
| Cip1) | | | |
| stem cell growth factor; lymphocyte | 1606 | 174 | 152 |
| secreted C-type lectin | | | |
| CD4 antigen (p55) | 5608 | 172 | 143 |
| PCTAIRE protein kinase 1 | 6334 | 161 | 78 |
| Rho GTPase activating protein 6 | 1035 | 160 | 220 |
| G protein-coupled receptor 37 (endothelin | 6318 | 158 | 35 |
| receptor type B-like) | | | |
| syndecan 4 (amphiglycan, ryudocan) | 3154 | 157 | 147 |
| PCTAIRE protein kinase 3 | 8050 | 155 | 159 |
| empty spiracles (Drosophila) homolog 2 | 2084 | 150 | 207 |
| transglutaminase 1 (K polypeptide | 6674 | 150 | 101 |
| epidermal type I, protein-glutamine-gamma- | | | |
| glutamyltransferase) | | | |
| potassium voltage-gated channel, | 1335 | 147 | 62 |
| subfamily G, member 2 | | | |
| aldehyde dehydrogenase 4 (glutamate | 5985 | 147 | 207 |
| gamma-semialdehyde dehydrogenase; | | | |
| pyrroline-5-carboxylate dehydrogenase) | | | |
| E1A binding protein p300 | 1433 | 144 | 197 |
| LIM homeobox transcription factor 1, beta | 8211 | 143 | 89 |
| eukaryotic translation elongation factor 2 | 4536 | 134 | 150 |
| mitogen-activated protein kinase kinase | 5462 | 129 | 92 |
| kinase 10 | | | |
| PPAR(gamma) angiopoietin related protein | 3074 | 129 | 56 |
| homeo box A5 | 7037 | 128 | 87 |
| CD63 antigen (melanoma 1 antigen) | 7769 | 124 | 91 |
| nuclear receptor coactivator 3 | 4181 | 119 | 84 |
| CD68 antigen | 443 | 118 | 256 |
| transmembrane 4 superfamily member 7 | 1571 | 117 | 29 |
| pancreatic polypeptide | 2735 | 116 | 136 |
| endothelin type b receptor-like protein 2 | 6656 | 114 | 74 |
| neurogenin 1 | 5772 | 113 | 91 |
| insulin receptor substrate 2 | 6016 | 110 | 130 |
| glutamate receptor, ionotropic, N-methyl D- | 4702 | 109 | 58 |
| asparate-associated protein 1 (glutamate | | | |
| binding) | | | |
| neurotrophin 5 (neurotrophin 4/5) | 8259 | 108 | 89 |
| pyrimidinergic receptor P2Y, G-protein | 3857 | 105 | 65 |
| coupled, 6 | | | |
| empty spiracles (Drosophila) homolog 1 | 2040 | 103 | 87 |
| chromobox homolog 2 (Drosophila Pc | 2832 | 101 | 139 |
| class) | | | |
| heart and neural crest derivatives | 2146 | 92 | 129 |
| expressed 1 | | | |
| transglutaminase 2 (C polypeptide, protein- | 7731 | 92 | 74 |
| glutamine-gamma-glutamyltransferase) | | | |
| enolase 1, (alpha) | 1389 | 92 | 205 |
| thyroid transcription factor 1 | 4519 | 89 | 60 |
| keratin 19 | 5923 | 88 | 223 |
| Deleted in split-hand/split-foot 1 region | 3641 | 87 | 104 |
| glutathione peroxidase 4 (phospholipid | 1874 | 84 | 72 |
| hydroperoxidase) | | | |
| aristaless (Drosophila) homeobox | 1783 | 81 | 103 |
| GATA-binding protein 4 | 659 | 80 | 110 |
| transcription factor 1, hepatic; LF-B1, | 712 | 80 | 136 |
| hepatic nuclear factor (HNF1), albumin | | | |
| proximal factor | | | |
| heat shock transcription factor 1 | 6708 | 79 | 57 |
| liver-specific bHLH-Zip transcription factor | 765 | 77 | 188 |
| liver-specific bHLH-Zip transcription factor | 765 | 77 | 188 |
| eukaryotic translation initiation factor 3, | 6280 | 76 | 94 |
| subunit 4 (delta, 44 kD) | | | |
| eukaryotic translation initiation factor 3, | 6280 | 76 | 94 |
| subunit 4 (delta, 44 kD) | | | |
| gamma-aminobutyric acid (GABA) A | 3720 | 70 | 178 |
| receptor, alpha 6 | | | |
| retinoic acid receptor, gamma | 5191 | 70 | 109 |
| homeo box D9 | 1776 | 69 | 34 |
| MAD1 (mitotic arrest deficient, yeast, | 1946 | 63 | 38 |
| homolog)-like 1 | | | |
| homeo box A4 | 7177 | 57 | 103 |
| Thy-1 cell surface antigen | 559 | 57 | 87 |
| talin | 603 | 53 | 84 |
| bone morphogenetic protein receptor, type | 332 | 53 | 45 |
| II (serine/threonine kinase) | | | |
| hepatocyte nuclear factor 3, alpha | 72 | 52 | 60 |
| ryanodine receptor 2 (cardiac) | 5244 | 47 | 40 |
| signal transducer and activator of | 3661 | 40 | 35 |
| transcription 6, interleukin-4 induced | | | |

TABLE 6

| SwissProt | Spot | geneName | day3 | day8 | day16 |
|---|---|---|---|---|---|
| P04270 | E19ab2 | actin; alpha; cardiac muscle | 1198.174 | 760 | 5955.077 |
| O95996 | N17ab2 | adenomatous polyposis coli like | 2708.542 | 5180 | 7594.139 |
| Q14697 | G02ef8 | alpha glucosidase II alpha subun [symbol] | 306.0691 | 350 | 775.3358 |
| P14209 | G23ab7 | antigen identified by monoclonal | 389.822 | 507 | 1238.43 |
| P30530 | G14ef5 | AXL receptor tyrosine kinase | 314.4639 | 288 | 634.5658 |
| O43770 | I23ab3 | B-cell CLL/lymphoma 7C | 700.573 | 1050 | 1504.52 |
| O43852 | P08ab3 | calumenin | 801.582 | 1290 | 4720.807 |
| O75718 | O24cd6 | cartilage associated protein | 430.4432 | 256 | 1090.59 |
| Q9Y3C0 | G14ef2 | CGI-116 protein | 181.0293 | 301 | 416.7246 |
| P78369 | G09cd6 | claudin 10 | 1176.087 | 742 | 2598.595 |
| P08572 | P08ef6 | collagen; type IV; alpha 2 | 1130.23 | 1350 | 4684.425 |
| P11802 | B05ef5 | cyclin-dependent kinase 4 | 914.1579 | 978 | 2342.994 |
| Q16555 | O07ab4 | dihydropyrimidinase-like 2 | 114.6165 | 164 | 921.1473 |
| P33316 | H23ab5 | dUTP pyrophosphatase | 177.2032 | 238 | 541.5382 |
| P07992 | K20ef6 | excision repair cross-complemen [symbol] | 362.031 | 415 | 907.7933 |
| O75636 | B14cd3 | ficolin (collagen/fibrinogen doma [symbol] | 1081.127 | 1010 | 4327.606 |
| Q9UBA6 | B19ef4 | G8 protein | 370.6842 | 578 | 1489.206 |
| Q9Y5P5 | P08ef7 | GDP-mannose pyrophosphoryla [symbol] | 171.1216 | 198 | 414.0862 |
| O75293 | E02ef7 | growth arrest and DNA-damage- [symbol] | 194.3626 | 203 | 386.6833 |
| P50152 | C05ab6 | guanine nucleotide binding prote [symbol] | 204.743 | 150 | 497.3097 |
| Q9NX09 | C05gh3 | HIF-1 responsive RTP801 | 348.8574 | 253 | 1032.929 |
| P05204 | J22ab7 | high-mobility group (nonhistone [symbol] | 2067.865 | 1120 | 6059.895 |
| Q9P0P2 | C17ef3 | homolog of yeast MOG1 | 325.8407 | 378 | 682.7568 |
| Q9NWF8 | L15gh2 | hypothetical protein FLJ10055 | 172.167 | 241 | 374.3 |
| Q9NWV4 | B17gh2 | hypothetical protein FLJ20580 | 221.4432 | 280 | 483.5381 |
| Q9P0S8 | A21ef3 | hypothetical protein HSPC195 | 153.824 | 165 | 330.9111 |
| Q9BWS9 | P04gh5 | hypothetical protein MGC3234 | 196.9154 | 161 | 427.2066 |
| Q9BSK0 | K21gh8 | hypothetical protein MGC4415 | 156.4692 | 117 | 684.3425 |
| Q9Y6M1 | P09cd6 | IGF-II mRNA-binding protein 2 | 135.2566 | 153 | 408.5101 |
| Q9NQX7 | H02gh7 | integral membrane protein 3 | 478.2728 | 481 | 1102.244 |
| P08648 | E23ef7 | integrin; alpha 5 (fibronectin rece [symbol] | 183.4629 | 124 | 478.7266 |
| O00410 | J24ab6 | karyopherin (importin) beta 3 | 326.6874 | 196 | 629.7656 |
| P07195 | N16ab6 | lactate dehydrogenase B | 1003.971 | 1200 | 5593.079 |
| P09382 | N22ab6 | lectin; galactoside-binding; solub [symbol] | 19170.52 | 35400 | 78086.75 |
| O75427 | P20ab6 | leucine-rich repeat protein; neur [symbol] | 174.9605 | 231 | 543.3883 |
| Q16553 | A15ab7 | lymphocyte antigen 6 complex; I [symbol] | 425.8933 | 494 | 938.4504 |
| O75900 | O05cd4 | matrix metalloproteinase 23A | 381.2184 | 297 | 851.7221 |
| Q9UNF1 | H16ef7 | melanoma antigen; family D; 2 | 193.8125 | 219 | 659.9701 |
| P13995 | J16cd6 | methylene tetrahydrofolate dehy [symbol] | 321.4878 | 226 | 759.6021 |
| O00265 | G19cd8 | microtubule-associated protein; [symbol] | 189.7616 | 234 | 377.5576 |
| P27361 | B13ef5 | mitogen-activated protein kinase | 358.0872 | 382 | 720.3119 |
| P24844 | E18cd6 | myosin; light polypeptide 9; regul [symbol] | 5480.451 | 7380 | 22518.95 |
| Q9NVD4 | J10gh2 | N-acetylneuraminic acid phosph [symbol] | 153.4762 | 167 | 390.6642 |
| Q9Y617 | N22ef8 | phosphoserine aminotransferase | 169.9503 | 250 | 1238.851 |
| O95356 | A11gh7 | pituitary tumor-transforming 3 | 237.2126 | 192 | 2276.181 |
| Q9Y5X6 | G04cd6 | plasma glutamate carboxypeptid [symbol] | 169.6268 | 142 | 366.6202 |
| Q15113 | G20ab8 | procollagen C-endopeptidase en [symbol] | 497.3967 | 402 | 2628.947 |
| O15460 | D20cd4 | procollagen-proline; 2-oxoglutar [symbol] | 262.1395 | 433 | 756.4003 |
| O43556 | I19gh1 | sarcoglycan; epsilon | 172.7191 | 215 | 349.304 |
| P50454 | M17ab6 | serine (or cysteine) proteinase in | 339.1762 | 612 | 4091.161 |
| Q92853 | A23cd8 | similar to vaccinia virus HindIII K [symbol] | 444.062 | 553 | 997.4551 |
| Q16658 | P20cd1 | singned-like (fascin homolog; sea | 262.7872 | 479 | 1472.155 |
| Q9Y4Y8 | E18cd7 | Sm protein F | 257.2026 | 278 | 548.9705 |
| Q9BVH9 | D18gh7 | thioredoxin related protein | 522.0262 | 481 | 1903.964 |
| Q9HBB0 | L08gh8 | Thy-1 co-transcribed | 327.3502 | 374 | 1110.527 |
| P01033 | P09ef7 | tissue inhibitor of metalloprotein [symbol] | 1236.457 | 1500 | 13029.41 |
| Q15582 | L20ef6 | transforming growth factor; beta- [symbol] | 1221.083 | 2060 | 8540.86 |
| Q01995 | M07cd2 | transgelin | 3307.821 | 3880 | 18534.27 |
| Q13641 | B05ab2 | trophoblast glycoprotein | 479.6488 | 426 | 1399.786 |
| P06468 | G08cd2 | tropomyosin 2 (beta) | 339.2292 | 589 | 13203.05 |
| P51784 | M21cd4 | ubiquitin specific protease 11 | 158.0478 | 194 | 491.1246 |
| P03996 | E17ab2 | actin; alpha 2; smooth muscle; a [symbol] | 412.1187 | 1940 | 41801.34 |
| P08123 | P02ef6 | collagen; type I; alpha 2 | 186.5666 | 576 | 14565.36 |
| P24821 | A05ef7 | hexabrachion (tenascin C; cytota | 355.495 | 918 | 2371.983 |
| Q9NVA2 | F13gh3 | hypothetical protein FLJ10849 | 374.1496 | 861 | 3471.789 |
| P40261 | M09cd1 | nicotinamide N-methyltransferase | 1060.579 | 2310 | 5683.533 |
| Q15063 | C01ef7 | osteoblast specific factor 2 (fascl [symbol] | 276.634 | 795 | 8328.222 |
| P09486 | A09ef7 | secreted protein; acidic; cysteine | 404.2839 | 2760 | 63837.2 |
| P35625 | K20ef5 | tissue inhibitor of metalloprotein [symbol] | 216.254 | 619 | 10078.17 |
| Q9H2L5 | G06gh8 | AD037 protein | 361.2297 | 681 | 158.8121 |
| P15121 | C18ab2 | aldo-keto reductase family 1; me [symbol] | 2342.744 | 5380 | 2337.052 |
| P08582 | B14ef5 | antigen p97 (melanoma associat [symbol] | 299.1375 | 595 | 80.80392 |
| P20292 | E06ab2 | arachidonate 5-lipoxygenase-act [symbol] | 100.0016 | 687 | 203.2911 |
| O15342 | A13ab3 | ATPase; H+ transporting; lysoso [symbol] | 6063.171 | 16500 | 6160.831 |
| Q9BXJ0 | H18gh6 | C1q and tumor necrosis factor re [symbol] | 180.2978 | 350 | 154.1441 |
| Q9NRJ3 | P09gh4 | CC chemokine CCL28 | 250.0021 | 1830 | 763.2866 |
| P05305 | F17ab5 | endothelin 1 | 866.4561 | 1810 | 403.431 |
| P09341 | A04ab6 | GRO1 oncogene (melanoma gro [symbol] | 933.2938 | 4770 | 1006.711 |
| Q92730 | O14ef4 | GTP-binding protein | 653.8575 | 2760 | 542.7528 |
| Q9H5K0 | L10gh5 | hypothetical protein FLJ23360 | 151.2029 | 296 | 130.3196 |
| P18564 | E21ef7 | integrin; beta 6 | 540.0362 | 1180 | 233.0055 |
| P14316 | B16ef6 | interferon regulatory factor 2 | 252.3603 | 472 | 255.7047 |
| P40305 | O20ab7 | interferon; alpha-inducible protei [symbol] | 117.3152 | 766 | 218.2471 |
| P05161 | B21cd5 | interferon-stimulated protein; 15 | 302.3796 | 983 | 326.6346 |
| P42701 | A06ef7 | interleukin 12 receptor; beta 1 | 1227.347 | 22100 | 1333.521 |
| P09237 | J15ef1 | matrix metalloproteinase 7 (matri | 9598.545 | 87300 | 19849.69 |
| P16860 | O13ab8 | natriuretic peptide precursor B | 4340.214 | 12900 | 1260.906 |
| P21359 | L16ab7 | neurofibromin 1 (neurofibromato [symbol] | 132.483 | 292 | 155.0822 |
| P02775 | B07ef7 | pro-platelet basic protein (includ [symbol] | 33.98656 | 1400 | 265.2119 |
| Q9UDQ9 | J22gh4 | SBBI26 protein | 121.5886 | 1080 | 238.8624 |
| O75635 | P14cd3 | serine (or cysteine) proteinase in | 232.6336 | 3890 | 326.2581 |
| P02735 | A11cd3 | serum amyloid A1 | 496.9578 | 17400 | 957.6141 |
| O60635 | F10cd5 | tetraspan 1 | 321.8888 | 843 | 177.6479 |
| P50591 | C09cd4 | tumor necrosis factor (ligand) su [symbol] | 536.7773 | 1550 | 476.2305 |
| Q03169 | I10ef7 | tumor necrosis factor; alpha-indu | 366.7502 | 1260 | 326.6536 |
| Q9C075 | N11ef8 | type I intermediate filament cytok | 848.9829 | 4200 | 401.4854 |
| P02248 | A01cd3 | ubiquitin C | 155.6735 | 892 | 62.04595 |
| O95497 | A15cd5 | vanin 1 | 309.1556 | 1840 | 510.7208 |
| Q14202 | B15cd5 | zinc finger protein 261 | 199.6246 | 1040 | 247.9161 |
| P15514 | M02ef7 | amphiregulin (schwannoma-deri [symbol] | 103.1419 | 405 | 65.66067 |
| Q9BZL9 | I21gh8 | B aggressive lymphoma gene | 126.3951 | 211 | 84.56927 |
| P30991 | D14ef5 | chemokine (C—X—C motif); recept [symbol] | 64.37115 | 260 | 120.2328 |
| P29400 | H13ab3 | collagen; type IV; alpha 5 (Alport | 53.50943 | 374 | 141.1609 |
| Q9UK22 | H22cd7 | F-box only protein 2 | 87.59822 | 217 | 120.3781 |
| Q16769 | O15cd8 | glutaminyl-peptide cyclotransfera | 89.90252 | 195 | 113.0683 |
| P02261 | M06gh6 | H2A histone family; member I | 65.46028 | 263 | 126.664 |
| P20769 | D03ef1 | immunoglobulin heavy constant [symbol] | 101.245 | 176 | 102.5687 |
| P52945 | I12ab6 | insulin promoter factor 1; homeo [symbol] | 74.60504 | 351 | 86.42558 |
| Q14496 | F21cd6 | interferon-induced protein 44 | 99.27101 | 286 | 98.28057 |
| P01583 | F15ef7 | interleukin 1; alpha | 103.386 | 375 | 66.6314 |
| P01584 | F17ef7 | interleukin 1; beta | 116.7675 | 415 | 119.0156 |
| Q9GZM1 | I20gh7 | NDRG family member 4 | 102.1328 | 348 | 101.569 |
| P25105 | G15ef5 | platelet-activating factor receptor | 97.43121 | 356 | 80.47935 |
| P58294 | O14gh8 | prokineticin 1 precursor | 79.98286 | 241 | 137.5167 |
| P23471 | A06ef6 | protein tyrosine phosphatase; re [symbol] | 85.55935 | 346 | 93.65845 |
| O88386 | D10ef3 | RAB10; member RAS oncogene | 123.2003 | 399 | 68.13944 |
| P10826 | I08gh6 | retinoic acid receptor; beta | 67.98871 | 241 | 120.9469 |
| O95786 | I24ef3 | RNA helicase | 112.9366 | 241 | 89.32396 |
| P02778 | O10ef7 | small inducible cytokine subfamil [symbol] | 65.03395 | 418 | 86.09019 |
| P80162 | F14cd1 | small inducible cytokine subfamil [symbol] | 54.00821 | 311 | 129.2639 |
| P43005 | N23ef5 | solute carrier family 1 (neuronal/ [symbol] | 99.30514 | 189 | 110.8119 |
| P01375 | O09ef6 | tumor necrosis factor (TNF supe [symbol] | 138.6153 | 386 | 75.13436 |
| Q16890 | G04cd2 | tumor protein D52-like 1 | 103.5477 | 191 | 98.01862 |
| Q9H949 | K11gh5 | WW45 protein | 111.8812 | 218 | 79.54574 |
| Q9H4G4 | O06gh5 | 17 kD fetal brain protein | 88.61746 | 223 | 532.2979 |
| Q14040 | B15ef1 | collagen; type VI; alpha 1 | 38.27203 | 209 | 4724.413 |
| P12110 | B17ef1 | collagen; type VI; alpha 2 | 88.00282 | 164 | 3835.637 |
| P02751 | K15ef1 | fibronectin 1 | 4.441362 | 152 | 4171.603 |
| P14652 | N23ab6 | homeo box B2 | 62.71629 | 291 | 3571.974 |
| P08476 | D15ef7 | inhibin; beta A (activin A; activin | 64.38161 | 318 | 854.6532 |
| P17936 | D07ab6 | insulin-like growth factor binding | 38.22268 | 349 | 1302.669 |
| Q9Y4K0 | P18ab6 | lysyl oxidase-like 2 | 75.91549 | 260 | 2525.644 |
| P03956 | N05ef7 | matrix metalloproteinase 1 (inter [symbol] | 106.2193 | 203 | 590.2622 |
| P39900 | N15ef7 | matrix metalloproteinase 12 (ma [symbol] | 18.2367 | 381 | 1219.953 |
| P55001 | G09ab7 | microfibrillar-associated protein 2 | 99.33548 | 526 | 3302.631 |
| Q9NYR0 | O07gh7 | SH3-domain kinase binding prot [symbol] | 34.69271 | 197 | 656.4675 |
| O43623 | D06ef4 | snail homolog 2 (Drosophila) | 96.88079 | 292 | 3028.508 |
| P09936 | I13cd4 | ubiquitin carboxyl-terminal ester [symbol] | 48.30052 | 219 | 1523.166 |
| Q02952 | J11ab2 | A kinase (PRKA) anchor protein | 103.9756 | 364 | 229.1446 |
| Q99541 | I19ab2 | adipose differentiation-related p [symbol] | 67.38509 | 182 | 146.1347 |
| P54284 | N17ab3 | calcium channel; voltage-depend | 106.3248 | 227 | 254.9633 |
| Q13269 | G14ab5 | cAMP responsive element bindin | 100.6639 | 193 | 217.7521 |
| Q9NPF2 | M02ef4 | chondroitin 4-sulfotransferase | 133.8375 | 192 | 303.01 |
| Q9H7A5 | F12gh7 | chromosome 20 open reading fra | 98.71521 | 179 | 208.0134 |
| Q9H9Q2 | L23gh5 | COP9 constitutive photomorpho [symbol] | 86.57682 | 115 | 184.3088 |
| P42771 | D17ef5 | cyclin-dependent kinase inhibitor | 43.35217 | 471 | 355.7426 |
| Q14650 | J20ef4 | cytoplasmic FMR1 interacting pr [symbol] | 132.5759 | 272 | 321.6348 |
| Q96IK6 | F14gh6 | DKFZP434C245 protein | 145.227 | 104 | 91.06477 |
| P12034 | D05ef7 | fibroblast growth factor 5 | 60.2377 | 259 | 317.6018 |
| Q93079 | M20gh6 | H2B histone family; member J | 144.6906 | 428 | 271.2983 |
| Q9P016 | A10ef8 | HSPC144 protein | 123.4601 | 348 | 399.4935 |
| Q9HBI5 | M09gh4 | HT021 | 116.3605 | 276 | 309.5798 |
| Q9BY45 | O24gh8 | HTPAP protein | 78.49388 | 109 | 177.2043 |
| P08397 | G18ab6 | hydroxymethylbilane synthase | 139.0857 | 188 | 260.8417 |
| Q9BUV0 | M06gh4 | hypothetical protein dJ465N24.2. | 68.40543 | 152 | 167.5592 |
| Q9H9A2 | N22gh6 | hypothetical protein DKFZp762 [symbol] | 82.54725 | 110 | 169.5379 |
| Q9NUV6 | P09gh3 | hypothetical protein FLJ11113 | 87.43713 | 178 | 136.6771 |
| Q9NXF7 | C20gh2 | hypothetical protein FLJ20280 | 66.49705 | 143 | 170.7367 |
| Q9H6V0 | M05gh5 | hypothetical protein FLJ21839 | 116.3704 | 258 | 323.3964 |
| Q9BUW5 | K21gh6 | hypothetical protein MGC4707 | 80.31127 | 170 | 203.9516 |
| Q9H765 | I15gh6 | hypothetical protein MGC5540 | 106.3949 | 220 | 206.6405 |
| Q14157 | N23gh1 | KIAA0144 gene product | 95.13957 | 147 | 147.7214 |
| Q9Y2D8 | A07ef8 | KIAA0923 protein | 84.1225 | 237 | 206.8269 |
| Q9H6Z3 | E24gh5 | kinesin family member 13A | 111.6382 | 206 | 238.8822 |
| Q99748 | J24ab8 | neurturin | 123.7486 | 150 | 246.4754 |
| Q9NVD7 | D11gh3 | parvin; alpha | 82.67948 | 140 | 230.66 |
| O43175 | H24cd6 | phosphoglycerate dehydrogenas | 98.81456 | 119 | 192.3326 |
| Q9Y253 | E24cd1 | polymerase (DNA directed); eta [symbol] | 108.2715 | 101 | 157.8762 |
| P24158 | M05ef5 | proteinase 3 (serine proteinase; [symbol] | 87.66238 | 280 | 172.8525 |
| Q9U8K7 | M21ef3 | RAB; member of RAS oncogene | 110.9871 | 189 | 313.4316 |
| Q13636 | K19cd7 | RAB31; member RAS oncogene | 76.36263 | 118 | 170.0352 |
| Q9UIC2 | L10ef2 | RNB6 | 80.78879 | 171 | 194.395 |
| Q9NVQ7 | K12gh3 | Sec61 alpha form 2 | 82.6036 | 143 | 162.0674 |
| O75368 | J08cd1 | SH3 domain binding glutamic aci | 92.82513 | 212 | 196.8753 |
| O95863 | N01cd2 | snail homolog 1 (Drosophila) | 76.58448 | 134 | 157.9807 |
| Q9UL01 | H01cd8 | squamous cell carcinoma antige [symbol] | 86.70353 | 111 | 176.4557 |
| Q16226 | N05cd6 | thioredoxin interacting protein | 111.1384 | 454 | 529.2322 |
| P07996 | A11ef7 | thrombospondin 1 | 79.00174 | 139 | 163.2593 |
| Q99081 | F10ef6 | transcription factor 12 (HTF4; he | 112.2294 | 181 | 256.0348 |
| O95922 | L06ef7 | tubulin tyrosine ligase-like 1 | 73.12945 | 245 | 253.9213 |
| P15036 | H18ef6 | v-ets erythroblastosis virus E26 [symbol] | 149.9259 | 114 | 83.55955 |
| O95337 | D04ef8 | weakly similar to glutathione per [symbol] | 69.55111 | 164 | 284.3593 |
| Q9H1B5 | M18gh5 | xylosyltransferase II | 81.87715 | 106 | 174.2539 |
| P55263 | K07ab2 | adenosine kinase | 891.6698 | 720 | 299.6337 |
| P51648 | A22ab2 | aldehyde dehydrogenase 3 famil | 1955.203 | 1390 | 507.3786 |
| P50995 | I08ab2 | annexin A11 | 4888.71 | 3870 | 1773.94 |
| P12429 | I14ab2 | annexin A3 | 9711.235 | 10800 | 1720.452 |
| P05026 | L22ab2 | ATPase; Na+/K+ transporting; b [symbol] | 3965.733 | 5290 | 1976.367 |
| P00751 | M07ab3 | B-factor; properdin | 2984.309 | 2460 | 1178.473 |
| Q9HA23 | E10gh6 | calponin like transmembrane dor | 480.5263 | 528 | 193.0456 |
| Q16170 | M17ab3 | carcinoembryonic antigen-relate [symbol] | 984.7628 | 1230 | 212.7712 |
| P25774 | P07ab5 | cathepsin S | 857.9751 | 776 | 228.9093 |
| P21926 | J13ef7 | CD9 antigen (p24) | 2011.966 | 3500 | 833.332 |
| Q9H2A7 | M19gh5 | chemokine (C—X—C motif) ligand 1 | 650.7961 | 842 | 235.8205 |
| O14493 | G21ab4 | claudin 4 | 1919.128 | 2480 | 799.5104 |
| P12277 | M06ab4 | creatine kinase; brain | 373.8119 | 617 | 193.0681 |
| Q9NYS7 | E20ef4 | CS box-containing WD protein | 596.6891 | 384 | 211.1821 |
| P53355 | D09ab5 | death-associated protein kinase | 1325.243 | 1260 | 558.9756 |
| O15205 | D17cd6 | diubiquitin | 7363.301 | 14700 | 2196.358 |
| Q9UFZ2 | C23ef8 | DKFZP564K247 protein | 1325.797 | 1060 | 313.5237 |
| P28562 | M22ab5 | dual specificity phosphatase 1 | 2077.188 | 1680 | 754.9876 |
| Q16690 | B21ab4 | dual specificity phosphatase 5 | 485.6475 | 305 | 155.0161 |
| Q9UJW0 | H10ef1 | dynactin 4 (p62) | 778.5079 | 662 | 335.9831 |
| | E13gh5 | ectonucleotide pyrophosphatase | 233.9728 | 378 | 183.3797 |
| P00533 | A06ef5 | epidermal growth factor receptor | 263.2516 | 334 | 182.4044 |
| Q13113 | J02cd5 | epithelial protein up-regulated in | 13324.19 | 15500 | 2641.961 |
| P28161 | M09ab6 | glutathione S-transferase M2 (m [symbol] | 1020.341 | 690 | 217.4433 |
| Q03013 | L06ab3 | glutathione S-transferase M4 | 333.2767 | 465 | 210.8696 |
| P52594 | A18ab7 | HIV-1 Rev binding protein | 439.4544 | 468 | 252.8058 |
| Q9NWT9 | D09gh2 | hypothetical protein FLJ20607 | 1062.388 | 757 | 368.3558 |
| Q9BZR4 | L12gh7 | hypothetical protein HT036 | 748.0681 | 844 | 160.1449 |
| Q9BW25 | A19gh6 | hypothetical protein MGC3101 | 315.4695 | 409 | 150.4768 |
| P16144 | I16ab6 | integrin; beta 4 | 490.7243 | 672 | 157.31 |
| P10145 | O22ef7 | interleukin 8 | 6902.736 | 13300 | 1783.339 |
| Q05084 | G10ab7 | islet cell autoantigen 1 (69 kD) | 600.5671 | 588 | 209.1329 |
| Q9Y484 | E14cd7 | JM5 protein | 1335.218 | 2010 | 938.9827 |
| P14923 | M23ef6 | junction plakoglobin | 1969.949 | 2610 | 912.3081 |
| P05783 | N06ef6 | keratin 18 | 17317.24 | 12500 | 2306.177 |
| P08727 | L06ab6 | keratin 19 | 4025.258 | 2800 | 631.8404 |
| P08729 | N14ef6 | keratin 7 | 11605.48 | 11100 | 1888.38 |
| P05787 | N16ef6 | keratin 8 | 11031.01 | 7880 | 1694.024 |
| O14782 | H22ab6 | kinesin family member 3C | 366.3528 | 652 | 242.2822 |
| O00515 | D05ab7 | ladinin 1 | 3562.632 | 2040 | 824.2377 |
| P80188 | D13ab7 | lipocalin 2 (oncogene 24p3) | 5234.435 | 9070 | 664.0466 |
| Q9UQ53 | G02ef3 | mannosyl (alpha-1; 3-)-glycoprot [symbol] | 1485.546 | 931 | 398.5069 |
| P80294 | D15gh6 | metallothionein 1H | 327.8489 | 366 | 208.8705 |
| O75394 | M10cd5 | mitochondrial ribosomal protein [symbol] | 441.6574 | 646 | 213.7295 |
| Q9Y376 | H18ef2 | MO25 protein | 860.3225 | 1410 | 550.0258 |
| P15941 | H22ab7 | mucin 1; transmembrane | 2330.649 | 1250 | 487.1995 |
| Q16301 | H11cd4 | myelin transcription factor 2 | 2433.02 | 2560 | 545.0513 |
| Q99836 | M07ab7 | myeloid differentiation primary re | 1088.39 | 1110 | 413.4864 |
| P24001 | H08cd4 | natural killer cell transcript 4 | 49232.84 | 71300 | 13531.68 |
| Q9C002 | O12gh8 | normal mucosa of esophagus sp [symbol] | 8605.554 | 17700 | 4423.686 |
| P25963 | A16ef1 | nuclear factor of kappa light poly | 938.908 | 1270 | 605.6118 |
| P00491 | L18ab7 | nucleoside phosphorylase | 820.4015 | 500 | 250.4586 |
| P49763 | O20ef7 | placental growth factor; vascular | 5604.053 | 12600 | 1723.953 |
| Q9Y342 | A13ef2 | plasmolipin | 942.5595 | 983 | 300.2833 |
| P43490 | H04cd5 | pre-B-cell colony-enhancing fact [symbol] | 2261.72 | 1730 | 651.3251 |
| Q99988 | B23ef7 | prostate differentiation factor | 1470.061 | 2930 | 681.8854 |
| Q9UJY1 | B05ef3 | protein kinase H11 | 1836.927 | 2080 | 544.441 |
| Q9H1C7 | O10gh8 | putative nuclear protein ORF1-F [symbol] | 11454.74 | 14800 | 3062.014 |
| P51149 | A21ef6 | RAB7; member RAS oncogene f [symbol] | 3422.13 | 5250 | 2267.107 |
| P51151 | J22cd4 | RAB9A; member RAS oncogene | 1882.687 | 1580 | 721.6194 |
| P15153 | C17ef6 | ras-related C3 botulinum toxin s [symbol] | 383.0769 | 418 | 213.0301 |
| P52566 | I19ef1 | Rho GDP dissociation inhibitor ( [symbol] | 986.1501 | 788 | 352.0789 |
| P01011 | I08ef7 | serine (or cysteine) proteinase in | 40450.89 | 43700 | 10172.44 |
| P05120 | P07ef7 | serine (or cysteine) proteinase in | 24922.82 | 38200 | 2879.818 |
| O14508 | M13ef6 | STAT induced STAT inhibitor-2 | 1511.844 | 1490 | 420.453 |
| Q9Y6N5 | G13gh4 | sulfide dehydrogenase like (yeas | 823.7665 | 871 | 284.0637 |
| O00161 | C23cd4 | synaptosomal-associated protein | 1077.833 | 1340 | 687.0432 |
| P31431 | F24cd1 | syndecan 4 (amphiglycan; ryudo [symbol] | 2109.871 | 3080 | 887.6687 |
| O15533 | B16ef7 | TAP binding protein (tapasin) | 313.1585 | 424 | 162.3501 |
| P17987 | E11ef5 | t-complex 1 | 495.6787 | 298 | 157.6175 |
| O14746 | O20ef6 | telomerase reverse transcriptase | 264.8687 | 373 | 151.5845 |
| P30408 | C06ef1 | transmembrane 4 superfamily m [symbol] | 16658.44 | 17400 | 6051.147 |
| Q9HCN3 | E07gh5 | transmembrane protein 8 (five m [symbol] | 947.8492 | 1010 | 462.0955 |
| Q16149 | B15cd1 | transporter 1; ATP-binding casse | 862.1561 | 1100 | 480.8685 |
| Q9Y2A9 | C22ef3 | UDP-GlcNac:betaGal beta-1; 3-N | 774.5506 | 436 | 153.9485 |
| O60625 | N10cd3 | vesicle-associated membrane p [symbol] | 2480.868 | 2680 | 584.39 |
| P07948 | A21ab7 | v-yes-1 Yamaguchi sarcoma vira | 512.646 | 443 | 170.1742 |
| Q9P2N4 | M10gh4 | a disintegrin-like and metalloprot [symbol] | 159.7274 | 227 | 72.96867 |
| P47895 | C04ab2 | aldehyde dehydrogenase 1 famil [symbol] | 1247.008 | 2370 | 146.6644 |
| P04233 | G09ef1 | CD74 antigen (invariant polypept [symbol] | 229.4566 | 174 | 86.99566 |
| Q9NZ31 | L12gh4 | chromosome 20 open reading fra [symbol] | 345.4771 | 375 | 137.4415 |
| O96002 | G07cd5 | chromosome × open reading frar [symbol] | 401.521 | 375 | 130.7318 |
| O94907 | J15gh6 | dickkopf homolog 1 (Xenopus la [symbol] | 150.3215 | 200 | 84.53571 |
| Q92796 | K06ef5 | discs; large (Drosophila) homolo [symbol] | 161.9581 | 154 | 84.90819 |
| Q13115 | O17ab4 | dual specificity phosphatase 4 | 533.5415 | 730 | 146.312 |
| P29317 | F02ef5 | EphA2 | 267.722 | 279 | 82.15254 |
| P29323 | D10ef5 | EphB2 | 269.1596 | 232 | 74.35016 |
| Q9UKF9 | H10cd7 | ets homologous factor | 603.0262 | 587 | 102.0333 |
| Q9NPD3 | P22gh2 | exosome component Rrp41 | 273.527 | 300 | 49.72784 |
| O43524 | G12ab4 | forkhead box O3A | 262.2734 | 169 | 102.6435 |
| P21217 | M16ab3 | fucosyltransferase 3 (galactoside | 189.3354 | 257 | 60.24803 |
| O75205 | D24ef2 | G protein-coupled receptor; famil [symbol] | 621.7284 | 382 | 90.93956 |
| O75712 | A03ef1 | gap junction protein; beta 3; 31 k [symbol] | 235.3139 | 402 | 145.5466 |
| Q92908 | D12ab4 | GATA binding protein 6 | 293.117 | 199 | 93.59788 |
| O95210 | P05cd4 | genethonin 1 | 331.9855 | 241 | 112.69 |
| O95395 | K21cd5 | glucosaminyl (N-acetyl) transfera [symbol] | 297.4236 | 274 | 45.82301 |
| P48506 | F22ab5 | glutamate-cysteine ligase; cataly [symbol] | 299.1944 | 243 | 134.9982 |
| Q9UI98 | F08ef8 | hqp0256 protein | 357.587 | 336 | 106.803 |
| Q9H6D8 | D18gh5 | hypothetical protein FLJ22362 | 542.6184 | 595 | 127.4299 |
| P05362 | I02ab6 | intercellular adhesion molecule 1 | 411.0131 | 424 | 67.80016 |
| Q07627 | J20gh7 | keratin associated protein 1.1 | 288.3566 | 303 | 40.28571 |
| Q9BYQ7 | D10gh8 | keratin associated protein 4.10 | 493.4934 | 605 | 96.51137 |
| O75071 | F15gh1 | KIAA0494 gene product | 240.2512 | 167 | 91.77931 |
| Q9UPQ2 | G22ef8 | KIAA1100 protein | 224.7248 | 349 | 135.6811 |
| O43896 | H10cd6 | kinesin family member 1C | 159.6982 | 170 | 73.44924 |
| Q13887 | L05ab5 | Kruppel-like factor 5 (intestinal) | 494.1259 | 376 | 97.43054 |
| O00312 | D22cd3 | MAP kinase-interacting serine/th [symbol] | 147.9629 | 150 | 86.60695 |
| O15264 | H07ab8 | mitogen-activated protein kinase | 213.7425 | 160 | 76.32069 |
| Q9UHA4 | P10gh1 | mitogen-activated protein kinase | 268.8101 | 244 | 72.50042 |
| P21397 | F23ef6 | monoamine oxidase A | 427.3495 | 480 | 30.42645 |
| P55196 | N07ab7 | myeloid/lymphoid or mixed-linea [symbol] | 164.9238 | 168 | 78.62952 |
| Q12965 | I24ab7 | myosin IE | 212.4935 | 276 | 122.0015 |
| Q9HBW1 | O10gh7 | NAG14 protein | 417.9837 | 396 | 86.84398 |
| Q9H2W4 | D20gh6 | neural precursor cell expressed; | 361.6431 | 463 | 75.41197 |
| Q9NRR3 | O06gh4 | non-kinase CDc42 effector protei [symbol] | 194.8075 | 327 | 117.0936 |
| P23511 | M19ab8 | nuclear transcription factor Y; al [symbol] | 239.2485 | 226 | 132.3351 |
| Q99650 | D14cd4 | oncostatin M receptor | 337.1614 | 233 | 141.0848 |
| P04085 | O16ef7 | platelet-derived growth factor alp [symbol] | 441.7554 | 463 | 124.4793 |
| Q13048 | N06cd2 | pregnancy specific beta-1-glycop [symbol] | 149.7737 | 203 | 84.53571 |
| P10586 | E11ef6 | protein tyrosine phosphatase; re [symbol] | 466.2876 | 405 | 136.6718 |
| O95200 | N22ab8 | retinoic acid receptor responder [symbol] | 437.2899 | 315 | 72.62859 |
| Q9NVX8 | P17ef3 | Rho GTPase activating protein 8 | 425.2848 | 479 | 109.2541 |
| Q15418 | N14ef5 | ribosomal protein S6 kinase; 90 k | 299.528 | 361 | 78.38666 |
| Q15434 | F10ab8 | RNA binding motif; single strand [symbol] | 454.6438 | 328 | 145.2438 |
| Q15437 | O10cd6 | Sec23 homolog B (S. cerevisiae) | 261.2005 | 235 | 126.9582 |
| O60679 | D15ef5 | serum-inducible kinase | 202.0368 | 194 | 62.20916 |
| Q12890 | N06ef5 | SFRS protein kinase 1 | 350.4057 | 278 | 113.0428 |
| Q12971 | L03cd6 | sialyltransferase | 247.3141 | 329 | 96.91002 |
| P78556 | M02cd2 | small inducible cytokine subfamil [symbol] | 308.0437 | 382 | 59.31325 |
| O75751 | F14ef4 | solute carrier family 22 (extraneu [symbol] | 164.6419 | 238 | 66.83593 |
| Q9UM01 | D08cd4 | solute carrier family 7 (cationic a [symbol] | 201.657 | 260 | 55.89528 |
| Q9Y5X1 | D10ef2 | sorting nexin 9 | 219.1839 | 240 | 108.9101 |
| P08842 | D19ab3 | steroid sulfatase (microsomal); a | 436.4255 | 414 | 136.2997 |
| O43760 | G09cd5 | synaptogyrin 2 | 391.0545 | 238 | 118.1167 |
| O75674 | N15cd5 | target of myb1-like 1 (chicken) | 276.5234 | 285 | 54.32353 |
| P01135 | K04ef7 | transforming growth factor; alpha | 215.9023 | 160 | 59.63082 |
| Q9NT70 | K13gh3 | transmembrane protein vezatin | 365.9385 | 401 | 129.2607 |
| O60656 | G13gh3 | UDP glycosyltransferase 1 family | 546.3937 | 438 | 27.51254 |
| Q9NZ42 | N18gh4 | uncharacterized hematopoietic s [symbol] | 196.9377 | 213 | 115.2737 |
| O76080 | E14cd4 | zinc finger protein 216 | 314.9706 | 211 | 74.56541 |
| Q13015 | H11ab2 | ALL1-fused gene from chromoso [symbol] | 329.6061 | 1730 | 1752.9 |
| P04083 | I06ab2 | annexin A1 | 1114.486 | 8240 | 8292.245 |
| O60592 | B14ab2 | Arg/Abl-interacting protein ArgBF | 1576.114 | 3320 | 2017.836 |
| Q07814 | E04ef6 | BCL2-associated × protein | 430.8903 | 862 | 1101.457 |
| Q14201 | E17cd7 | BTG family; member 3 | 197.8608 | 451 | 419.4378 |
| Q9NR00 | J21gh4 | chromosome 8 open reading fra [symbol] | 2766.931 | 7310 | 3283.111 |
| Q9BUW7 | K19gh6 | chromosome 9 open reading fra [symbol] | 228.4878 | 650 | 551.4272 |
| O95401 | N04cd4 | cofactor required for Sp1 transcri [symbol] | 186.3494 | 395 | 588.6133 |
| Q00535 | B09ef5 | cyclin-dependent kinase 5 | 269.7495 | 476 | 432.1176 |
| P21741 | E17ab7 | midkine (neurite growth-promotin | 2991.463 | 15400 | 9802.656 |
| Q9Y291 | J15ef2 | mitochondrial ribosomal protein [symbol] | 177.4689 | 330 | 365.4484 |
| O75376 | E17cd6 | nuclear receptor co-repressor 1 | 375.3679 | 1270 | 727.3836 |
| P03973 | N10cd1 | secretory leukocyte protease inhi [symbol] | 592.855 | 2070 | 4153.51 |
| O15427 | F06cd4 | solute carrier family 16 (monocar [symbol] | 228.58 | 753 | 785.8975 |
| P16949 | D11ab7 | stathmin 1/oncoprotein 18 | 728.2643 | 2630 | 1984.807 |
| Q9NZ86 | J08gh3 | uncharacterized bone marrow p [symbol] | 311.3754 | 945 | 536.938 |
| O00748 | H07cd4 | carboxylesterase 2 (intestine; live [symbol] | 1080.553 | 220 | 253.3623 |
| Q13630 | M01cd3 | tissue specific transplantation an | 622.3905 | 287 | 255.576 |
| P00505 | C14ab6 | glutamic-oxaloacetic transamina [symbol] | 494.0876 | 356 | 260.2263 |
| P50440 | A09ab5 | glycine amidinotransferase (L-ar [symbol] | 1737.906 | 395 | 261.2318 |
| P29622 | A06cd1 | serine (or cysteine) proteinase in [symbol] | 461.3518 | 189 | 264.9574 |
| O14646 | C17ab4 | chromodomain helicase DNA bin [symbol] | 867.7631 | 180 | 273.1703 |
| P48307 | I10cd4 | tissue factor pathway inhibitor 2 | 920.3007 | 366 | 277.6619 |
| Q15125 | D08cd6 | emopamil binding protein (sterol | 897.4306 | 230 | 282.4317 |
| P19387 | B13ab8 | polymerase (RNA) II (DNA direct | 196.4282 | 155 | 282.8549 |
| Q9P0K7 | H08gh6 | retinoic acid induced 14 | 177.983 | 275 | 283.8007 |
| P30086 | E04ab8 | prostatic binding protein | 634.2686 | 462 | 300.6211 |
| Q13045 | C06ab5 | flightless I homolog (Drosophila) | 622.7072 | 501 | 306.0777 |
| O95084 | G10cd7 | protease; serince: 23 | 1096.756 | 246 | 314.746 |
| P49770 | L11ef4 | eukaryotic translation initiation fa [symbol] | 731.6826 | 547 | 329.7361 |
| Q13948 | J02ef6 | cut-like 1: CCAAT displacement [symbol] | 222.2758 | 349 | 330.5149 |
| Q30201 | H05ab3 | hemochromatosis | 205.1607 | 291 | 339.4607 |
| P52895 | I16gh6 | aido-keto reductase family 1; me [symbol] | 4001.572 | 263 | 339.7988 |
| Q9Y5N1 | M24cd7 | histamine receptor H3 | 526.5133 | 203 | 341.1301 |
| P43378 | N23ab8 | protein tyrosine phosphatase; no | 1360.729 | 445 | 368.4734 |
| P35414 | O11ab2 | angiotensin receptor-like 1 | 620.7039 | 406 | 395.8948 |
| Q14802 | K03cd1 | FXYD domain-containing ion tra [symbol] | 2174.215 | 711 | 402.87 |
| Q9Y266 | F16cd6 | nuclear distribution gene C hom [symbol] | 745.3985 | 447 | 408.3037 |
| P30043 | O21ab3 | biliverdin reductase B (flavin red [symbol] | 877.6813 | 565 | 417.1383 |
| Q9NP73 | L06gh3 | uncharacterized hematopoietic s [symbol] | 804.2103 | 446 | 421.5183 |
| Q12972 | D17ab8 | protein phosphatase 1; regulator | 251.1796 | 358 | 425.52 |
| Q9Y296 | N21ef2 | PTD009 protein | 738.5086 | 424 | 436.6114 |
| Q02750 | K02ef7 | mitogen-activated protein kinase | 810.206 | 508 | 441.4249 |
| P42226 | G23cd2 | signal transducer and activator 0 | 892.6025 | 516 | 450.4229 |
| P31947 | M07ef6 | stratifin | 2941.197 | 741 | 452.267 |
| P46597 | J12ab2 | acetylserotonin O-methyltransfer | 2225.944 | 437 | 466.9229 |
| O14745 | J24cd4 | solute carrier family 9 (sodium/h [symbol] | 1844.719 | 642 | 504.4269 |
| Q9NS67 | D24gh1 | G protein-coupled receptor 27 | 1464.475 | 381 | 595.0192 |
| Q9UKL5 | J07cd8 | programmed cell death 6 interact | 995.6375 | 886 | 605.9272 |
| Q9UQL3 | B11ef2 | liver-specific bHLH-Zip transcript | 825.7324 | 1150 | 606.7901 |
| Q9NZZ1 | P18ef2 | hypothetical protein HSPC138 | 397.5554 | 450 | 618.4035 |
| Q9Y3Q3 | H03cd7 | integral type I protein | 526.2023 | 355 | 631.4284 |
| Q9NSN3 | C10gh4 | similar to aspartate beta hydroxy | 355.1305 | 273 | 667.4052 |
| Q9Y5Z0 | B12cd7 | beta-site APP-cleaving enzyme 2 | 1469.687 | 960 | 680.3343 |
| | C21gh7 | H2B histone family; member B | 511.589 | 855 | 733.4035 |
| O14713 | M11cd5 | integrin cytoplasmic domain-ass [symbol] | 446.3806 | 746 | 737.9643 |
| P46095 | P11ab4 | G protein-coupled receptor 6 | 2336.841 | 460 | 760.4217 |
| Q9BV10 | K07gh6 | hypothetical protein MGC3136 | 1761.627 | 653 | 779.7338 |
| Q99653 | O08cd7 | calcium binding protein P22 | 1713.549 | 1430 | 793.2383 |
| P41584 | P16gh1 | polymerase (RNA) II (DNA direct | 560.1066 | 892 | 821.9856 |
| P11940 | E06ab8 | poly(A) binding protein; cytoplas [symbol] | 1738.585 | 669 | 827.8343 |
| Q00341 | G21ab6 | high density lipoprotein binding [symbol] | 545.5844 | 444 | 866.5328 |
| Q16795 | K08ab7 | NADH dehydrogenase (ubiquino [symbol] | 1582.872 | 1190 | 873.0478 |
| Q13082 | E05ab7 | microtubule-associated protein 4 | 1217.396 | 472 | 873.8683 |
| P36405 | D16ab2 | ADP-ribosylation factor-like 3 | 508.3031 | 547 | 939.7264 |
| Q9NXH2 | C04gh2 | hypothetical protein FLJ20254 | 515.6731 | 467 | 959.9781 |
| Q9Y323 | D21ef2 | hypothetical protein HSPC177 | 838.9663 | 1630 | 976.2242 |
| P48059 | I12ab7 | LIM and senescent cell antigen- [symbol] | 560.9674 | 918 | 1003.945 |
| Q9P0M9 | M02ef2 | mitochondrial ribosomal protein [symbol] | 579.0083 | 936 | 1024.359 |
| Q13813 | E07cd2 | spectrin; alpha; non-erythrocytic | 2261.592 | 886 | 1070.984 |
| Q9Y379 | H07ef2 | CGI-69 protein | 2556.713 | 1900 | 1174.107 |
| Q13247 | O13gh1 | splicing factor; arginine/serine-ri [symbol] | 718.1614 | 1210 | 1194.951 |
| P14416 | D03ef6 | dopamine receptor D2 | 814.5351 | 745 | 1299.03 |
| Q06323 | A15ef7 | proteasome (prosome; macropai [symbol] | 1491.803 | 2350 | 1386.719 |
| Q9NR31 | H17gh4 | SAR1 protein | 752.6343 | 886 | 1386.849 |
| P05451 | C07cd3 | regenerating islet-derived 1 alph [symbol] | 75644.12 | 1690 | 1394.425 |
| P04075 | C12ab2 | aldolase A; fructose-bisphosphat | 4534.199 | 1590 | 1470.967 |
| Q15904 | A19ab3 | ATPase; H+ transporting; lysoso [symbol] | 833.0193 | 1380 | 1471.799 |
| P15559 | G24ef7 | NAD(P)H dehydrogenase; quino [symbol] | 537.0892 | 303 | 1486.45 |
| O00303 | L20cd3 | eukaryotic translation initiation fa [symbol] | 1243.805 | 636 | 1556.448 |
| P56705 | M01gh7 | wingless-type MMTV integration | 3589.752 | 1590 | 1635.805 |
| P39210 | I04ef7 | MpV17 transgene; murine homo [symbol] | 990.336 | 894 | 1723.06 |
| Q9UKL9 | N03ef4 | aldo-keto reductase family 1; me [symbol] | 16311.31 | 2840 | 1896.102 |
| O43516 | E12cd3 | Wiskott-Aldrich syndrome protein | 1162.511 | 1190 | 1979.246 |
| Q9Y470 | D01ef7 | stem cell growth factor; lymphoc [symbol] | 1346.693 | 1110 | 2049.542 |
| P29312 | H12cd4 | tyrosine 3-monooxygenase/trypt [symbol] | 5187.766 | 4710 | 2331.854 |
| Q9Y5R8 | P24gh4 | MUM2 protein | 1596.032 | 1750 | 2568.113 |
| P17676 | D01ab5 | CCAAT/enhancer binding protein | 6064.068 | 2880 | 2723.688 |
| O95362 | F11gh6 | differentially expressed in hemat [symbol] | 10407.52 | 2680 | 2912.158 |
| O60417 | C11gh7 | S100 calcium binding protein A1 [symbol] | 6955.642 | 5690 | 3097.579 |
| P19623 | E17cd2 | spermidine synthase | 1644.266 | 1020 | 3417.978 |
| Q9P039 | P02ef7 | FXYD domain-containing ion tra [symbol] | 2410.08 | 2120 | 4486.029 |
| Q9BUF5 | F05gh8 | tubulin beta-5 | 2252.623 | 1580 | 4730.755 |
| P00995 | C23cd2 | serine protease inhibitor; Kazal [symbol] | 67989.21 | 11300 | 5416.175 |
| P11142 | E22ef7 | heat shock 70 kD protein 8 | 11154.99 | 7840 | 5433.843 |
| P10620 | E16ef7 | microsomal glutathione S-transfe [symbol] | 6706.736 | 2430 | 5571.411 |
| Q13347 | N02cd3 | eukaryotic translation initiation fa [symbol] | 19633.03 | 5210 | 5660.757 |
| P15880 | B16cd1 | ribosomal protein S2 | 4356.01 | 2060 | 5964.993 |
| Q9HCU0 | E24gh4 | tumor endothelial marker 1 prec [symbol] | 3139.145 | 2600 | 7148.075 |
| Q04941 | O16ab8 | proteolipid protein 2 (colonic epit [symbol] | 17951.73 | 11800 | 8567.736 |
| Q9UPI1 | M14cd2 | small nuclear ribonucleoprotein [symbol] | 4625.515 | 8280 | 8998.787 |
| P13987 | B18ef7 | CD59 antigen p18-20 (antigen id | 17383.17 | 19200 | 10453.43 |
| P15951 | N04cd2 | protease; serine; 3 (trypsin 3) | 74972.46 | 7610 | 11417.5 |
| P04687 | P12cd1 | tubulin; alpha; ubiquitous | 15943.82 | 7260 | 15413.44 |
| P04687 | H12cd1 | tubulin; alpha; ubiquitous | 15832.93 | 7590 | 24085.35 |
| Q9UI09 | L02gh4 | 13 kDa differentiation-associated | 1754.698 | 1990 | 2889.148 |
| Q9Y6H1 | E23ef2 | 16.7 Kd protein | 5687.333 | 4050 | 5837.94 |
| Q9P0U1 | C07gh3 | 6.2 kd protein | 2323.402 | 1990 | 3847.586 |
| P80095 | N10ab7 | 6-pyruvoyl-tetrahydropterin synt [symbol] | 3223.922 | 2620 | 1569.69 |
| Q92747 | F04ab2 | actin related protein 2/3 complex | 4894.401 | 3990 | 3030.018 |
| O15144 | F08ab2 | actin related protein 2/3 complex | 13976.76 | 21400 | 17206.31 |
| O15145 | F10ab2 | actin related protein 2/3 complex | 3065.881 | 3190 | 1793.813 |
| P02570 | A01cd7 | actin; beta | 66304.81 | 45400 | 38777.92 |
| P02570 | H12cd7 | actin; beta | 64949.74 | 42400 | 44598.66 |
| P02570 | P12cd7 | actin; beta | 21347.36 | 12200 | 15408.61 |
| P02571 | A06ab6 | actin; gamma 1 | 69165.16 | 59100 | 65677.15 |
| P12814 | E23ab2 | actinin; alpha 1 | 2400.607 | 2160 | 2638.011 |
| O43707 | G05ab2 | actinin; alpha 4 | 6999.138 | 3810 | 3499.234 |
| P53999 | C05cd7 | activated RNA polymerase II tran [symbol] | 4478.986 | 4320 | 6009.318 |
| P18848 | I03ef5 | activating transcription factor 4 ( [symbol] | 7160.888 | 5420 | 7668.286 |
| P53680 | A07ef1 | adaptor-related protein complex [symbol] | 2255.235 | 2530 | 4102.633 |
| P29274 | A18ef5 | adenosine A2a receptor | 1990.124 | 2960 | 2004.596 |
| P54819 | A04ab2 | adenylate kinase 2 | 2997.607 | 2520 | 2851.739 |
| Q01518 | O18cd6 | adenylyl cyclase-associated pro [symbol] | 6946.788 | 11000 | 7171.205 |
| P32889 | C03ef6 | ADP-ribosylation factor 1 | 6040.796 | 7220 | 7907.02 |
| P18085 | P21ab2 | ADP-ribosylation factor 4 | 4777.904 | 8880 | 8342.165 |
| P26437 | B02ab2 | ADP-ribosylation factor 5 | 3739.477 | 4430 | 3984.845 |
| Q9BZX4 | L08gh6 | AKAP-binding sperm protein rop [symbol] | 2345.455 | 2780 | 2500.118 |
| P11766 | K01ab2 | alchocol dehydrogenase 5 (class [symbol] | 2220.156 | 1990 | 2854.318 |
| P05067 | N03ef6 | amyloid beta (A4) precursor prot [symbol] | 2846.85 | 4180 | 3918.595 |
| Q06481 | K22ab2 | amyloid beta (A4) precursor-like | 2117.652 | 3230 | 2290.191 |
| P07355 | I12ab2 | annexin A2 | 74767.39 | 112000 | 97217.17 |
| P08758 | I18ab2 | annexin A5 | 4487.905 | 4270 | 7134.202 |
| P55064 | O08ab2 | aquaporin 5 | 10851.57 | 14200 | 11650.24 |
| Q13520 | O10ab2 | aquaporin 6; kidney specific | 2404.748 | 2350 | 2457.278 |
| P32391 | D19ab2 | ARP3 actin-related protein 3 ho [symbol] | 4291.823 | 4280 | 3941.05 |
| O43776 | E18ab7 | asparaginyl-tRNA synthetase | 2570.968 | 3010 | 2854.989 |
| P24539 | N22ab2 | ATP synthase; H+ transporting; [symbol] | 2908.697 | 3800 | 3228.203 |
| P48201 | P04ab2 | ATP synthase; H+ transporting; [symbol] | 5694.182 | 4370 | 4857.955 |
| P05496 | N24ab2 | ATP synthase; H+ transporting; [symbol] | 2176.378 | 1760 | 2400.575 |
| Q06055 | P02ab2 | ATP synthase; H+ transporting; [symbol] | 5784.579 | 3530 | 7732.552 |
| O75947 | P14ab2 | ATP synthase; H+ transporting; [symbol] | 2668.028 | 2520 | 1881.974 |
| P56385 | P06ab2 | ATP synthase; H+ transporting; [symbol] | 2682.798 | 2090 | 2616.726 |
| P56134 | P10ab2 | ATP synthase; H+ transporting; [symbol] | 11587.91 | 17200 | 14181.18 |
| P18859 | P08ab2 | ATP synthase; H+ transporting; [symbol] | 3879.939 | 4840 | 5176.585 |
| O75964 | P16ab2 | ATP synthase; H+ transporting; [symbol] | 2021.591 | 2630 | 2009.026 |
| P25705 | N14ab2 | ATP synthase; H+ transporting; [symbol] | 3657.512 | 2410 | 2967.207 |
| P06576 | N16ab2 | ATP synthase; H+ transporting; [symbol] | 4072.978 | 3280 | 2557.962 |
| P36542 | N18ab2 | ATP synthase; H+ transporting; [symbol] | 2479.5 | 2030 | 2436.284 |
| P48047 | P18ab2 | ATP synthase; H+ transporting; [symbol] | 4222.314 | 4100 | 3541.047 |
| Q01814 | P01ef5 | ATPase; Ca++ transporting; plas [symbol] | 3157.447 | 3180 | 3242.707 |
| Q99437 | A11ab3 | ATPase; H+ transporting; lysoso [symbol] | 2495.618 | 3870 | 2087.291 |
| P36543 | A09ab3 | ATPase; H+ transporting; lysoso [symbol] | 1854.198 | 2920 | 1659.749 |
| O75348 | A15ab3 | ATPase; H+ transporting; lysoso [symbol] | 2734.484 | 5660 | 2721.878 |
| Q16864 | A21ab3 | ATPase; H+ transporting; lysoso [symbol] | 6452.29 | 15900 | 7960.234 |
| P27449 | A05ab3 | ATPase; H+ transporting; lysoso [symbol] | 17134.14 | 24300 | 13120.57 |
| O00244 | L14ab2 | ATX1 antioxidant protein 1 homo | 2833.957 | 3500 | 3977.547 |
| O14503 | M19ab3 | basic helix-loop-helix domain co [symbol] | 4978.916 | 4280 | 3254.631 |
| O60238 | L11ab5 | BCL2/adenovirus E1B 19 kD inte [symbol] | 1473.428 | 2420 | 3224.141 |
| P30536 | L21ab3 | benzodiazapine receptor (periph [symbol] | 1997.929 | 2890 | 2326.211 |
| P01884 | C23ab3 | beta-2-microglobulin | 64872 | 105000 | 36055.95 |
| P22004 | I13ab5 | bone morphogenetic protein 6 | 5036.985 | 4710 | 3780.005 |
| O95415 | B02ef3 | brain protein I3 | 7498.734 | 11200 | 5348.84 |
| O75531 | K11ab3 | Breakpoint cluster region protein | 2541.789 | 2740 | 2924.826 |
| P02593 | A10ab6 | calmodulin 2 (phosphorylase kin [symbol] | 1573.77 | 4130 | 4742.485 |
| P07384 | O24ef1 | calpain 1; (mu/l) large subunit | 3429.132 | 3420 | 2390.011 |
| P27797 | P05ab5 | calreticulin | 10259.42 | 9700 | 15437.35 |
| | N06gh8 | CaM-KII inhibitory protein | 20298.71 | 28200 | 21760.79 |
| P47756 | N14ab4 | capping protein (actin filament) n | 4348.079 | 4450 | 5015.377 |
| P15086 | H13ab5 | carboxypeptidase B1 (tissue) | 9766.062 | 27.1 | 26.97129 |
| P52952 | D22ab5 | cardiac-specific homeo box | 2859.197 | 1740 | 2282.546 |
| P13862 | M21ef1 | casein kinase 2; beta polypeptid [symbol] | 3674.921 | 3840 | 3873.228 |
| O14675 | I02ef6 | CASP8 and FADD-like apoptosis | 2622.586 | 2500 | 1151.661 |
| P35221 | C17ef7 | catenin (cadherin-associated pro [symbol] | 3813.516 | 4230 | 3300.738 |
| P07858 | E01ab5 | cathepsin B | 2480.797 | 3860 | 4485.422 |
| P48509 | K16ab4 | CD151 antigen | 7267.56 | 13700 | 8768.731 |
| P25063 | G22cd8 | CD24 antigen (small cell lung ca [symbol] | 21319.27 | 18200 | 8278.973 |
| P16070 | O21ef1 | CD44 antigen (homing function [symbol] | 16178.96 | 9640 | 5778.642 |
| P08962 | O18ab4 | CD63 antigen (melanoma 1 anti [symbol] | 9354.914 | 11600 | 10016.29 |
| O14519 | K04ef5 | CDK2-associated protein 1 | 4231.191 | 4180 | 4763.013 |
| P25763 | A12ab6 | cell division cycle 42 (GTP bindi [symbol] | 4588.819 | 2840 | 2852.186 |
| P49368 | N17cd2 | chaperonin containing TCP1; su [symbol] | 2356.627 | 2880 | 2222.361 |
| P48643 | H10ef4 | chaperonin containing TCP1; su [symbol] | 2238.933 | 2330 | 1561.625 |
| O00299 | M19ab6 | chloride intracellular channel [symbol] | 6811.55 | 7240 | 5727.121 |
| Q9Y2Q7 | N02cd8 | chromosome 11 open reading fr [symbol] | 4087.361 | 3800 | 5946.259 |
| P09496 | F15ab5 | clathrin; light polypeptide (Lca) | 5758.632 | 9260 | 5849.641 |
| O95832 | L04gh1 | claudin 1 | 3046.376 | 2450 | 918.0838 |
| O95471 | A13ef1 | claudin 7 | 2888.819 | 3350 | 1520.977 |
| P23528 | M20ab5 | cofillin 1 (non-musle) | 16021.95 | 18700 | 14063.43 |
| P16989 | L08ef6 | cold shock domain protein A | 2942.723 | 2530 | 2069.184 |
| P38936 | O21ef5 | cyclin-dependent kinase inhibitor | 3643.18 | 4250 | 3437.076 |
| Q9UII4 | B14ef1 | cyclin-E binding protein 1 | 26180.8 | 29800 | 39655.02 |
| P04080 | G16ab3 | cystatin B (stefin B) | 4312.168 | 5420 | 2370.13 |
| P21291 | J01ab5 | cysteine and glycine-rich protein | 2478.366 | 1710 | 2135.86 |
| O00622 | J15ef7 | cysteine-rich; angiogenic inducer | 2070.203 | 1620 | 2978.542 |
| P13073 | L19ab5 | cytochrome c oxidase subunit IV | 6219.628 | 8550 | 6571.848 |
| P12074 | K13gh1 | cytochrome c oxidase subunit VI [symbol] | 3253.24 | 5800 | 4244.557 |
| Q02221 | E17ab6 | cytochrome c oxidase subunit VI [symbol] | 2968.24 | 2820 | 4604.241 |
| P14854 | A20ab6 | cytochrome c oxidase subunit VII | 1991.91 | 2660 | 2380.795 |
| P09669 | E12ab6 | cytochrome c oxidase subunit VI [symbol] | 2580.598 | 2090 | 1927.002 |
| P14406 | G02ab5 | cytochrome c oxidase subunit VII | 4846.713 | 4500 | 4245.478 |
| O14548 | G23cd5 | cytochrome c oxidase subunit VII | 2182.954 | 2450 | 3040.586 |
| P24311 | K17ab5 | cytochrome c oxidase subunit VII | 3224.558 | 5150 | 3801.752 |
| P15954 | P04ab5 | cytochrome c oxidase subunit VII | 12335.74 | 12200 | 9883.688 |
| P10176 | B10ab5 | cytochrome c oxidase subunit VII | 3019.162 | 1900 | 3329.281 |
| O43293 | K19ab4 | death-associated protein kinase | 2535.403 | 1850 | 2088.808 |
| P46966 | G14ef6 | defender against cell death 1 | 7921.682 | 11200 | 9546.896 |
| Q09753 | G01ab6 | defensin; beta 1 | 4071.982 | 7630 | 2153.638 |
| P18282 | F23ab2 | destrin (actin depolymerizing fac [symbol] | 7729.126 | 7570 | 6083.408 |
| P07108 | H04gh1 | diazepam binding inhibitor (GAB [symbol] | 2541.011 | 1730 | 2995.768 |
| P31689 | D18ab7 | DnaJ (Hsp40) homolog; subfamil [symbol] | 2491.051 | 2930 | 1789.662 |
| Q9Y5T4 | I10cd8 | DNAJ domain-containing | 2324.933 | 2520 | 2397.896 |
| Q9Y463 | G15cd5 | dual-specificity tyrosine-(Y)-phos | 8211.965 | 6870 | 5987.39 |
| Q9NP97 | C02ef8 | dynein light chain 2A | 4080.949 | 4160 | 5365.928 |
| Q15701 | L08cd3 | dynein; cytoplasmic; light polype [symbol] | 4492.679 | 5800 | 4332.161 |
| P78545 | O16ab4 | E74-like factor 3 (ets domain tra [symbol] | 3781.196 | 2780 | 1455.944 |
| O60869 | A03cd4 | endothelial differentiation-related | 5515.692 | 5680 | 4090.527 |
| Q14259 | L06ab4 | enhancer of rudimentary homolo [symbol] | 3593.608 | 3950 | 5175.131 |
| P06733 | C16ab4 | enolase 1; (alpha) | 12539.97 | 7460 | 9955.024 |
| P22712 | D11gh6 | enolase 1; (alpha) | 6900.857 | 4290 | 5016.695 |
| P04720 | O07ab6 | eukaryotic translation elongation | 36222.34 | 23200 | 23485.94 |
| P26641 | A10ab4 | eukaryotic translation elongation | 6863.565 | 6540 | 11607.61 |
| P13639 | I15ab5 | eukaryotic translation elongation | 4023.855 | 2310 | 3360.073 |
| O15372 | L24cd3 | eukaryotic translation initiation fa [symbol] | 2870.027 | 1930 | 2391.869 |
| O75821 | L22cd3 | eukaryotic translation initiation fa [symbol] | 3009.075 | 2670 | 3333.728 |
| Q64252 | L11ab6 | eukaryotic translation initiation fa [symbol] | 2755.438 | 1540 | 2127.874 |
| O15371 | L18cd3 | eukaryotic translation initiation fa [symbol] | 4273.919 | 4030 | 3558.838 |
| P04765 | A22ab4 | eukaryotic translation initiation fa [symbol] | 7716.222 | 7170 | 6363.169 |
| Q14240 | H13ab4 | eukaryotic translation initiation fa [symbol] | 2091.816 | 2190 | 2519.322 |
| P10159 | A18ab5 | eukaryotic translation initiation fa [symbol] | 9023.244 | 5240 | 5651.752 |
| Q9NQT4 | H05gh4 | exosome component Rrp46 | 2098.514 | 2590 | 2304.48 |
| P02794 | C12ab6 | ferritin; heavy polypeptide 1 | 35912.81 | 48700 | 42075.22 |
| P02792 | M10ab3 | ferritin; light polypeptide | 44225.86 | 29600 | 17849.2 |
| Q05472 | B02ab4 | Finkel-Biskis-Reilly murine sarco | 10322.94 | 10400 | 9704.642 |
| P20071 | F08ab3 | FK506 binding protein 1A (12 kD) | 2741.389 | 1870 | 1414.969 |
| P50395 | J02ab4 | GDP dissociation inhibitor 2 | 2287.713 | 1580 | 2291.27 |
| P48507 | E12ab5 | glutamate-cysteine ligase; modif [symbol] | 154.1773 | 76.2 | 5937.375 |
| P36969 | D14ab5 | glutathione peroxidase 4 (phosp [symbol] | 3397.2 | 4860 | 3108.036 |
| P09211 | L10ab3 | glutathione S-transferase pi | 6392.334 | 8670 | 4067.789 |
| P04406 | A01cd8 | glyceraldehyde-3-phosphate deh [symbol] | 28809.37 | 38300 | 44655.67 |
| P04406 | H12cd8 | glyceraldehyde-3-phosphate deh [symbol] | 37562.25 | 60200 | 57135.41 |
| P04406 | P12cd8 | glyceraldehyde-3-phosphate deh [symbol] | 12053.5 | 14900 | 19061.62 |
| P43304 | H01ab3 | glycerol-3-phosphate dehydroge [symbol] | 3450.634 | 2340 | 3276.264 |
| P41250 | F02ef7 | glycyl-tRNA synthetase | 4333.439 | 3630 | 6248.604 |
| P04895 | K17ef5 | GNAS complex locus | 7755.858 | 9290 | 9107.558 |
| P24522 | B03ef6 | growth arrest and DNA-damage- [symbol] | 2792.429 | 6130 | 3396.736 |
| Q92847 | M24ab5 | growth hormone secretagogue re [symbol] | 2827.902 | 2100 | 2597.123 |
| P04901 | B01ef1 | guanine nucleotide binding prote [symbol] | 5734.337 | 7610 | 5853.112 |
| P25388 | E20cd6 | guanine nucleotide binding prote [symbol] | 22430.28 | 15300 | 19055.7 |
| P06351 | G08ab6 | H3 histone; family 3B (H3.3B) | 2448.264 | 2770 | 2042.864 |
| P04792 | A16ef5 | heat shock 27 kD protein 1 | 41952.14 | 34500 | 31970.86 |
| P11021 | H01ef1 | heat shock 70 kD protein 5 (glucc [symbol] | 3226.183 | 1600 | 4101.742 |
| P38646 | O19ab7 | heat shock 70 kD protein 9B (mor [symbol] | 2851.743 | 2180 | 2220.509 |
| Q9UK76 | J14ef1 | hematological and neurological [symbol] | 26342.33 | 35400 | 41099.58 |
| P09651 | F12ab7 | heterogeneous nuclear ribonucle [symbol] | 2522.253 | 1650 | 2524.082 |
| Q9Y4J5 | I23ef1 | heterogeneous nuclear ribonucle [symbol] | 3825.912 | 2370 | 3770.457 |
| P09429 | F10ab7 | high-mobility group (nonhistone [symbol] | 2534.273 | 1660 | 3502.047 |
| P49773 | M03ef6 | histidine triad nucleotide binding | 6025.978 | 7790 | 7379.267 |
| Q92769 | N05ab4 | histone deacetylase 2 | 2454.324 | 2220 | 1789.508 |
| P17693 | F08ab7 | HLA-G histocompatibility antigen | 2048.009 | 2780 | 1844.177 |
| P17483 | C15gh2 | homeo box B4 | 2892.488 | 3490 | 2902.69 |
| Q9UL99 | C01cd8 | hyaluronoglucosaminidase 4 | 3623.461 | 2050 | 2189.656 |
| Q9GZT3 | L18gh7 | hypothetical protein DC50 | 3343.922 | 3750 | 4298.676 |
| Q9NWY5 | O12gh2 | hypothetical protein FLJ20533 | 3104.471 | 5460 | 6184.227 |
| Q9UI30 | P08ef2 | hypothetical protein HSPC152 | 3000.217 | 4060 | 4396.862 |
| Q9BQB6 | P08gh5 | hypothetical protein IMAGE3455 [symbol] | 3599.509 | 5410 | 8687.307 |
| Q9BWJ5 | P18gh7 | hypothetical protein MGC3133 | 2074.073 | 2520 | 2719.671 |
| Q9Y683 | N11ef2 | hypothetical protein MGC8721 | 2425.475 | 2730 | 2939.253 |
| Q16665 | P01ef6 | hypoxia-inducible factor 1; alpha | 3544.753 | 2960 | 1526.935 |
| O75353 | G04ef6 | immediate early response 3 | 4564.408 | 4690 | 3129.721 |
| Q16270 | J15ab6 | insulin-like growth factor binding | 6185.622 | 21800 | 18047.34 |
| Q9Y287 | A03gh2 | integral membrane protein 2B | 6908.997 | 10500 | 10014.47 |
| P56537 | D16ab6 | integrin beta 4 binding protein | 2594.316 | 3270 | 1568.747 |
| P26006 | D08ab6 | integrin; alpha 3 (antigen CD49C [symbol] | 2669.777 | 2740 | 1339.712 |
| P05556 | G01ef7 | integrin; beta 1 (fibronectin recep [symbol] | 6743.428 | 5390 | 6511.579 |
| Q01628 | C09gh7 | interferon induced transmembra [symbol] | 14426.62 | 14800 | 8827.997 |
| Q12905 | C10ab7 | interleukin enhancer binding fact [symbol] | 3045.484 | 2420 | 2596.915 |
| Q13907 | A24ab7 | isopentenyl-diphosphate delta is [symbol] | 2958.139 | 1660 | 2475.365 |
| P33947 | K03cd7 | KDEL (Lys-Asp-Glu-Leu) endopl [symbol] | 1667.246 | 1250 | 3924.993 |
| P00338 | D15ab7 | lactate dehydrogenase A | 44081.29 | 34500 | 42150.03 |
| P08865 | G21ef7 | laminin receptor 1 (67 kD; riboso [symbol] | 8548.245 | 6070 | 9921.409 |
| Q08380 | D17ab7 | lectin; galactoside-binding; solub [symbol] | 2294.491 | 2850 | 3014.02 |
| O14949 | F17ef3 | low molecular mass ubiquinone- [symbol] | 2942.579 | 3550 | 3637.463 |
| Q15012 | F20ef4 | lysosomal-associated protein tra [symbol] | 2815.002 | 4690 | 5453.634 |
| P14174 | J05ef7 | macrophage migration inhibitory | 5544.722 | 6700 | 6763.718 |
| P49006 | M05ef6 | macrophage myristoylated alanir [symbol] | 5857.382 | 10400 | 8986.315 |
| P30463 | G10ab6 | major histocompatibility complex; | 2701.757 | 4890 | 2255.929 |
| P10321 | A01cd2 | major histocompatibility complex; | 8949.086 | 17000 | 10196.88 |
| P10321 | H12cd2 | major histocompatibility complex; | 7187.905 | 21600 | 11111.54 |
| P10321 | P12cd2 | major histocompatibility complex; | 13121.35 | 16800 | 11725.68 |
| P13747 | M24ab7 | major histocompatibility complex; | 2540.911 | 2790 | 2053.351 |
| Q95HC0 | P02gh6 | major histocompatibility complex; | 5067.294 | 4140 | 3295.115 |
| P13640 | D13gh6 | metallothionein 1G | 6180.376 | 4750 | 2940.683 |
| P80297 | D17gh6 | metallothionein 1X | 4881.982 | 5310 | 5233.749 |
| P02795 | L04ab7 | metallothionein 2A | 6620.235 | 7330 | 8067.274 |
| O14880 | E04ab7 | microsomal glutathione S-transfe [symbol] | 5023.542 | 7940 | 4177.165 |
| Q9NYZ2 | M22ef2 | mitochondrial solute carrier | 2488.07 | 2850 | 2240.824 |
| P26038 | K05ab7 | moesin | 4260.51 | 4580 | 3801.298 |
| O15329 | O12ef1 | mucin 6; gastric | 5574.093 | 5950 | 6685.047 |
| P16475 | J02gh1 | myosin; light polypeptide 6; alkal | 48122.62 | 45200 | 48882.54 |
| P19105 | N03cd6 | myosin; light polypeptide; regula [symbol] | 7920.873 | 8150 | 7228.401 |
| Q9UK23 | D06ef1 | N-acetylglucosamine-1-phospho [symbol] | 1884.596 | 2680 | 2511.293 |
| O15239 | E22ab7 | NADH dehydrogenase (ubiquino [symbol] | 2859.738 | 5440 | 3719.503 |
| O00483 | J02ab7 | NADH dehydrogenase (ubiquino [symbol] | 5214.222 | 5540 | 9086.615 |
| O75438 | J04ab8 | NADH dehydrogenase (ubiquino [symbol] | 1628.024 | 2170 | 2842.647 |
| O95168 | J08ab8 | NADH dehydrogenase (ubiquino [symbol] | 2297.787 | 3270 | 2253.854 |
| O95298 | J12ab8 | NADH dehydrogenase (ubiquino [symbol] | 1909.371 | 2840 | 2888.281 |
| O43920 | J18ab8 | NADH dehydrogenase (ubiquino [symbol] | 6241.135 | 11500 | 8014.187 |
| Q13765 | H09ab7 | nascent-polypeptide-associated [symbol] | 2800.535 | 3390 | 3897.956 |
| Q15843 | K13cd1 | neural precursor cell expressed; | 3449.484 | 4710 | 4139.56 |
| Q9NX14 | C01gh3 | neuronal protein 17.3 | 3476.812 | 3150 | 5077.834 |
| Q9Y2R6 | F18gh6 | NICE-3 protein | 1763.974 | 2250 | 2329.521 |
| Q15668 | H13cd6 | Niemann-Pick disease; type C2 | 1516.592 | 3640 | 2828.255 |
| O14597 | J21ef4 | non-functional folate binding pro [symbol] | 2917.293 | 2170 | 3004.012 |
| P15531 | E02ef5 | non-metastatic cells 1; protein (N | 6653.515 | 6940 | 7836.897 |
| P22392 | L12cd2 | non-metastatic cells 2; protein (N | 4827.494 | 5680 | 4836.596 |
| O00746 | D04ef7 | non-metastatic cells 4; protein e [symbol] | 2211.058 | 2270 | 4300.604 |
| O75534 | B01cd4 | NRAS-related gene | 3478.657 | 3510 | 2737.425 |
| Q9H9A1 | C24gh6 | nuclear receptor co-repressor/H [symbol] | 5951.715 | 3290 | 2990.083 |
| Q9NPE3 | K15ef4 | nucleolar protein family A; memb [symbol] | 3543.239 | 7130 | 3639.205 |
| P54368 | G10ef1 | ornithine decarboxylase antizym [symbol] | 7212.068 | 6880 | 7782.339 |
| Q9UH52 | L23cd8 | over-expressed breast tumor pro [symbol] | 2335.527 | 2320 | 1884.264 |
| Q15070 | A09cd1 | oxidase (cytochrome c) assembly | 2161.529 | 2500 | 1436.483 |
| Q9H230 | G14gh5 | p53-induced protein PIGPC1 | 1823.554 | 3330 | 1443.914 |
| O60356 | M03cd8 | p8 protein (candidate of metasta [symbol] | 1774.942 | 1940 | 2740.792 |
| P78337 | M22ab8 | paired-like homeodomain transcr [symbol] | 15428.96 | 13300 | 14490.63 |
| Q9C086 | P20gh7 | PAP-1 binding protein | 2992.2 | 3610 | 5014.215 |
| Q15165 | D06ef6 | paraoxonase 2 | 1325.972 | 3740 | 2122.119 |
| P20962 | N13ab8 | parathymosin | 2345.869 | 2330 | 3489.616 |
| P23284 | I17ab8 | peptidylprolyl isomerase B (cyclo [symbol] | 5298.839 | 5860 | 8890.43 |
| Q06830 | E18ab8 | peroxiredoxin 1 | 2973.862 | 2450 | 2056.112 |
| P32119 | G04ef7 | peroxiredoxin 2 | 3141.718 | 3330 | 2921.471 |
| P30048 | N09ab2 | peroxiredoxin 3 | 2420.936 | 2010 | 1671.796 |
| Q13162 | N07ab2 | peroxiredoxin 4 | 2744.707 | 2080 | 3645.718 |
| P30044 | P15cd7 | peroxiredoxin 5 | 2205.14 | 3820 | 1714.041 |
| Q13492 | B13cd4 | phosphatidylinositol binding clat [symbol] | 2688.917 | 2690 | 1861.508 |
| P08237 | P02ab7 | phosphofructokinase; muscle | 2790.708 | 1980 | 2829.743 |
| P00558 | P06ab7 | phosphoglycerate kinase 1 | 5186.346 | 5130 | 6341.68 |
| P18669 | K14ab6 | phosphoglycerate mutase 1 (brai [symbol] | 1847.463 | 2730 | 2859.446 |
| Q14801 | B01cd8 | phosphoprotein enriched in astrc [symbol] | 23286.56 | 17400 | 19729.36 |
| O14832 | A04cd1 | phytanoyl-CoA hydroxylase (Ref [symbol] | 7333.104 | 7780 | 8341.917 |
| P53801 | L01ef1 | pituitary tumor-transforming 1 int [symbol] | 4385.308 | 5330 | 4172.137 |
| Q9HB21 | E19gh5 | pleckstrin homology domain-con [symbol] | 2318.08 | 3070 | 2907.381 |
| Q15365 | O09cd1 | poly(rC) binding protein 1 | 4617.635 | 3130 | 3156.644 |
| P52433 | B17ab8 | polymerase (RNA) II (DNA direct | 2157.353 | 3780 | 3657.763 |
| P52436 | L16gh1 | polymerase (RNA) II (DNA direct | 2161.398 | 2600 | 2709.495 |
| Q03052 | B19ab8 | POU domain; class 3; transcripti [symbol] | 3321.206 | 2650 | 2251.677 |
| | K20gh5 | PP1201 protein | 7127.293 | 6650 | 2688.636 |
| P40425 | G08ab8 | pre-B-cell leukemia transcription | 2817.191 | 2650 | 3413.81 |
| Q99471 | K06ab8 | prefoldin 5 | 1827.777 | 2160 | 2837.334 |
| Q9UHZ2 | E21ef8 | PRO1073 protein | 1872.208 | 3140 | 1690.101 |
| P07737 | A21cd1 | profilin 1 | 29650.38 | 48900 | 35394.8 |
| Q92740 | E03gh1 | prosaposin (variant Gaucher dis [symbol] | 4235.232 | 5370 | 4268.459 |
| P07478 | N02cd2 | protease; serine; 2 (trypsin 2) | 77359.27 | 42600 | 51746.68 |
| Q9UL46 | N09ab8 | proteasome (prosome; macropa [symbol] | 3682.153 | 4550 | 2712.172 |
| P25786 | L09ef7 | proteasome (prosome; macropa [symbol] | 2270.079 | 2570 | 2265.763 |
| P20618 | J17ab8 | proteasome (prosome; macropa [symbol] | 2835.653 | 3590 | 2820.916 |
| P49720 | J21ab8 | proteasome (prosome; macropa [symbol] | 1714.767 | 2100 | 2435.426 |
| P28070 | J23ab8 | proteasome (prosome; macropa [symbol] | 2193.138 | 1760 | 2224.325 |
| Q99436 | L03ab8 | proteasome (prosome; macropa [symbol] | 4179.694 | 3450 | 3072.098 |
| P08129 | G01ef6 | protein phosphatase 1; catalytic [symbol] | 2543.089 | 2200 | 2276.462 |
| O15355 | D07ab8 | protein phosphatase 1G (former [symbol] | 5896.408 | 4200 | 3717.85 |
| P30153 | A06ef3 | protein phosphatase 2 (formerly [symbol] | 3858.567 | 4100 | 2765.011 |
| Q15249 | H13ef5 | prothymosin; alpha (gene seque [symbol] | 11382.44 | 14500 | 9974.587 |
| Q9NQ11 | G03gh7 | putative ATPase | 2774.724 | 2460 | 2271.593 |
| P41567 | N06cd5 | putative translation initiation fact [symbol] | 7487.872 | 6600 | 8909.545 |
| Q15181 | L18gh1 | pyrophosphatase (inorganic) | 4914.897 | 4350 | 2667.605 |
| P14786 | M24ab8 | pyruvate kinase; muscle | 6305.222 | 8060 | 9000.504 |
| P57735 | G12gh4 | RAB25; member RAS oncogene | 3343.628 | 4890 | 1366.379 |
| P54725 | E21cd1 | RAD23 homolog A (S. cerevisiae | 2533.36 | 1790 | 1753.113 |
| P54727 | M10ef6 | RAD23 homolog B (S. cerevisiae | 4038.864 | 5410 | 4983.21 |
| O00538 | P16cd5 | RAS guanyl releasing protein 2 ( [symbol] | 2418.688 | 2310 | 1906.199 |
| P06749 | C21gh1 | ras homolog gene family; memb [symbol] | 2136.708 | 1940 | 3003.841 |
| P08134 | B16ab2 | ras homolog gene family; memb [symbol] | 4404.567 | 4490 | 2930.236 |
| | P06gh8 | ras-like protein VTS58635 | 5451.542 | 6580 | 9066.798 |
| P15154 | C01ef6 | ras-related C3 botulinum toxin s [symbol] | 10849.13 | 9720 | 10044.19 |
| Q15347 | B20cd6 | Ras-related GTP-binding protein | 1728.963 | 2380 | 2623.418 |
| O95197 | A06cd6 | reticulon 3 | 3712.756 | 3420 | 3833.411 |
| P09455 | F12ab8 | retinol binding protein 1; cellular | 2211.12 | 4480 | 2383.274 |
| P52565 | K13ef6 | Rho GDP dissociation inhibitor ( [symbol] | 3166.04 | 3200 | 3141.434 |
| P27635 | N19cd2 | ribosomal protein L10 | 13406.29 | 7100 | 11103.69 |
| P53025 | G22cd1 | ribosomal protein L10a | 19374.73 | 15100 | 19939.26 |
| P39026 | D07cd1 | ribosomal protein L11 | 14172.29 | 6760 | 10346.92 |
| P30050 | D09cd1 | ribosomal protein L12 | 38376.69 | 24500 | 38669.34 |
| P40429 | A01ef1 | ribosomal protein L13a | 23800.5 | 22200 | 28159.37 |
| P50914 | B18cd4 | ribosomal protein L14 | 18507.36 | 14600 | 16108.18 |
| P39030 | C13cd3 | ribosomal protein L15 | 8407.532 | 8100 | 10809.12 |
| P18621 | D19cd1 | ribosomal protein L17 | 8779.141 | 8440 | 10004.78 |
| Q07020 | D13cd1 | ribosomal protein L18 | 12168.43 | 10200 | 15619.17 |
| Q02543 | C15gh1 | ribosomal protein L18a | 24976.59 | 27200 | 28170.19 |
| P14118 | D15cd1 | ribosomal protein L19 | 6226.589 | 5530 | 5535.643 |
| P46778 | A13cd3 | ribosomal protein L21 | 11156.59 | 11800 | 16742.21 |
| P35268 | D17cd1 | ribosomal protein L22 | 16289.45 | 16200 | 18991.35 |
| P23131 | F11cd4 | ribosomal protein L23 | 20972.93 | 20400 | 25729.93 |
| P29316 | G09cd3 | ribosomal protein L23a | 6385.675 | 4840 | 5446.963 |
| P38663 | D21cd1 | ribosomal protein L24 | 7805.803 | 6180 | 5479.195 |
| P08526 | F01cd1 | ribosomal protein L27 | 12874.22 | 5280 | 10256.69 |
| P46776 | F05cd1 | ribosomal protein L27a | 16046.37 | 17000 | 20949.85 |
| P46779 | F07cd1 | ribosomal protein L28 | 40540.59 | 32400 | 45810.52 |
| P47914 | E23cd3 | ribosomal protein L29 | 3346.473 | 1960 | 1947.028 |
| P39023 | B21cd1 | ribosomal protein L3 | 13192.5 | 10500 | 13962.55 |
| P04645 | F03cd1 | ribosomal protein L30 | 17693.61 | 12300 | 21530.83 |
| P12947 | F09cd1 | ribosomal protein L31 | 9749.468 | 8980 | 12591.65 |
| P02433 | A15cd3 | ribosomal protein L32 | 22774.44 | 24400 | 30742.77 |
| P49207 | F11cd1 | ribosomal protein L34 | 13251.1 | 11500 | 13193.66 |
| P42766 | K16cd7 | ribosomal protein L35 | 10454.27 | 10500 | 10655.65 |
| P18077 | F13cd1 | ribosomal protein L35a | 14583.91 | 10700 | 8811.726 |
| P09896 | J06gh1 | ribosomal protein L36a | 8395.644 | 8780 | 12052.18 |
| P09896 | A19cd3 | ribosomal protein L36a-like | 5167.775 | 5750 | 4693.401 |
| P02403 | F15cd1 | ribosomal protein L37 | 31895.4 | 45000 | 41301.54 |
| P12751 | F17cd1 | ribosomal protein L37a | 18652.48 | 17300 | 19955.82 |
| P23411 | F19cd1 | ribosomal protein L38 | 5149.82 | 5570 | 7560.21 |
| P02404 | A17cd3 | ribosomal protein L39 | 14114.05 | 21300 | 22827.16 |
| P36578 | B23cd1 | ribosomal protein L4 | 14367.47 | 10400 | 10448.85 |
| P28751 | L06gh1 | ribosomal protein L41 | 35135.75 | 34300 | 40372.62 |
| P46777 | E21cd3 | ribosomal protein L5 | 12002.66 | 8880 | 10351.69 |
| Q02878 | D01cd1 | ribosomal protein L6 | 15861.7 | 7300 | 10119.01 |
| P18124 | D03cd1 | ribosomal protein L7 | 21230.67 | 15900 | 19538.32 |
| P11518 | G11cd3 | ribosomal protein L7a | 13385.5 | 8240 | 10586.53 |
| P25120 | D05cd1 | ribosomal protein L8 | 23580.43 | 18300 | 24766.14 |
| P32969 | B19cd1 | ribosomal protein L9 | 28853.42 | 32200 | 37288.41 |
| P46783 | H13cd1 | ribosomal protein S10 | 7453.342 | 7660 | 7511.766 |
| P04643 | H15cd1 | ribosomal protein S11 | 13429.85 | 15600 | 13623.52 |
| P25398 | H17cd1 | ribosomal protein S12 | 16170.73 | 10600 | 12294.27 |
| Q02546 | H19cd1 | ribosomal protein S13 | 13956.88 | 12600 | 15289.54 |
| P11174 | H21cd1 | ribosomal protein S15 | 21322.73 | 19600 | 23334.6 |
| P39027 | H23cd1 | ribosomal protein S15a | 16787.1 | 15100 | 21854.01 |
| P17008 | J01cd1 | ribosomal protein S16 | 26604.5 | 15700 | 24481.55 |
| P08708 | J03cd1 | ribosomal protein S17 | 13979.25 | 7990 | 15360.88 |
| P25232 | A17gh2 | ribosomal protein S18 | 12558.18 | 12600 | 20093.07 |
| P17075 | J07cd1 | ribosomal protein S20 | 21944.13 | 19900 | 31381.43 |
| P35265 | D24ef7 | ribosomal protein S21 | 14046.7 | 9470 | 9023.678 |
| P39028 | J09cd1 | ribosomal protein S23 | 12228.98 | 10900 | 14550.17 |
| P16632 | J11cd1 | ribosomal protein S24 | 12913.37 | 13100 | 12135.49 |
| P25111 | J13cd1 | ribosomal protein S25 | 6274.159 | 5630 | 5123.747 |
| P02383 | C01cd3 | ribosomal protein S26 | 26138.26 | 30900 | 19440.2 |
| P42677 | G01cd3 | ribosomal protein S27 (metallop [symbol] | 17556.94 | 12800 | 20383.13 |
| P14798 | B18cd1 | ribosomal protein S27a | 3284.551 | 3180 | 2757.252 |
| P25112 | J15cd1 | ribosomal protein S28 | 30384.24 | 32000 | 28529.21 |
| P30054 | J17cd1 | ribosomal protein S29 | 18454.55 | 20900 | 25539.27 |
| P23396 | H03cd1 | ribosomal protein S3 | 5913.682 | 2830 | 5523.561 |
| P49241 | O16ef5 | ribosomal protein S3A | 7154.088 | 6200 | 8592.248 |
| P12750 | A21cd3 | ribosomal protein S4; X-linked | 19821.56 | 16200 | 21778.05 |
| P22090 | A23cd3 | ribosomal protein S4; Y-linked | 2629.697 | 2330 | 2779.184 |
| P46782 | H05cd1 | ribosomal protein S5 | 15421.08 | 11000 | 17355.54 |
| P10660 | H07cd1 | ribosomal protein S6 | 46408.18 | 26100 | 40579.46 |
| P23821 | H09cd1 | ribosomal protein S7 | 18906.94 | 16200 | 14326.88 |
| P09058 | H11cd1 | ribosomal protein S8 | 15941.2 | 12300 | 20997.59 |
| P46761 | A01cd5 | ribosomal protein S9 | 6215.562 | 3930 | 5714.843 |
| P46781 | H12cd5 | ribosomal protein S9 | 8827.416 | 9710 | 15295.98 |
| P46781 | P12cd5 | ribosomal protein S9 | 9059.523 | 1790 | 5725.082 |
| P05387 | H01cd1 | ribosomal protein; large P2 | 16611.14 | 12700 | 17775.72 |
| P05388 | F21cd1 | ribosomal protein; large; P0 | 17127.41 | 12400 | 19746.88 |
| P05386 | F23cd1 | ribosomal protein; large; P1 | 2226.298 | 1880 | 3134.529 |
| Q9Y254 | C08ef3 | ring-box 1 | 2597.578 | 4260 | 3365.165 |
| Q9NWJ8 | A23gh3 | roundabout homolog 4; magic [symbol] | 1969.602 | 3940 | 2093.279 |
| P08206 | D06cd1 | S100 calcium binding protein A1 [symbol] | 38485.73 | 49800 | 31745.47 |
| P31949 | E09cd3 | S100 calcium binding protein A1 [symbol] | 8890.594 | 11100 | 5408.269 |
| P06703 | P17gh6 | S100 calcium binding protein A6 | 3403.146 | 4070 | 2088.478 |
| P55735 | J04ef4 | SEC 13-like 1 (S. cerevisiae) | 1695.152 | 1990 | 2362.541 |
| P38384 | I06ef3 | Sec61 gamma | 5653.955 | 7820 | 7254.423 |
| Q9NZJ3 | H06ef2 | selenoprotein T | 2923.813 | 2440 | 1325.491 |
| O15532 | H16cd1 | selenoprotein W; 1 | 2678.843 | 2970 | 3918.44 |
| Q13501 | J23cd4 | sequestosome 1 | 3214.395 | 3170 | 1164.17 |
| P35237 | L18ab8 | serine (or cysteine) proteinase in | 4203.074 | 8180 | 2936.72 |
| P05121 | P05ef7 | serine (or cysteine) proteinase in | 4454.56 | 7280 | 9179.863 |
| Q00587 | C14cd7 | serum constituent protein | 2480.698 | 2660 | 2044.608 |
| Q9H299 | P16gh7 | SH3 domain binding glutamic aci | 14473.6 | 17100 | 13798.02 |
| P37108 | E21cd2 | signal recognition particle 14 kD ( | 24326.27 | 35600 | 24319.59 |
| P49458 | E19cd2 | signal recognition particle 9 kD | 1874.967 | 2640 | 3141.422 |
| P43308 | G15cd2 | signal sequence receptor; beta ( [symbol] | 4209.147 | 4610 | 5876.451 |
| P51571 | O15gh1 | signal sequence receptor; delta ( | 2062.113 | 2330 | 2654.968 |
| O00422 | M17cd6 | sin3-associated polypeptide; 18 k | 2502.79 | 2610 | 2079.295 |
| O75918 | J10cd5 | small EDRK-rich factor 2 | 5927.595 | 7010 | 7926.059 |
| P13500 | C15cd3 | small inducible cytokine A2 (mon | 17077.97 | 38100 | 8786.323 |
| Q15356 | A09cd2 | small nuclear ribonucleoprotein [symbol] | 2743.428 | 2150 | 2819.855 |
| P14648 | A13cd2 | small nuclear ribonucleoprotein [symbol] | 899.6242 | 2800 | 2362.597 |
| P12236 | G01ef1 | solute carrier family 25 (mitochor | 7384.066 | 4760 | 6176.034 |
| Q00325 | K22ab8 | solute carrier family 25 (mitochor | 5074.308 | 3750 | 3779.577 |
| P32745 | N01cd1 | somatostatin receptor 3 | 4102.668 | 4170 | 4748.989 |
| P30626 | E13cd2 | sorcin | 1895.592 | 2790 | 2280.297 |
| Q01826 | F18ef6 | special AT-rich sequence bindin [symbol] | 26449.32 | 41300 | 24225.61 |
| P21673 | D12cd1 | spermidine/spermine N1-acetyl [symbol] | 8349.882 | 16500 | 6013.659 |
| P34991 | G05cd3 | S-phase kinase-associated prote | 3072.601 | 2990 | 2396.506 |
| P23246 | B09cd2 | splicing factor proline/glutamine [symbol] | 2694.629 | 2460 | 3348.423 |
| P35716 | C07cd2 | SRY (sex determining region Y)- [symbol] | 5019.988 | 4860 | 2125.089 |
| P35713 | E10ef4 | SRY (sex determining region Y)- [symbol] | 2575.791 | 3100 | 2024.386 |
| Q06945 | C05cd2 | SRY (sex determining region Y)- [symbol] | 2299.474 | 2210 | 1636.067 |
| P00441 | G20ef7 | superoxide dismutase 1; soluble | 4439.342 | 4930 | 3239.195 |
| P50502 | I18cd2 | suppression of tumorigenicity 13 | 2307.599 | 2530 | 2358.117 |
| Q9Y5Y6 | P20gh1 | suppression of tumorigenicity 14 | 2358.28 | 2700 | 1494.856 |
| P17600 | J06cd2 | synapsin I | 1969.157 | 2220 | 2010.908 |
| O43759 | G11cd5 | synaptogyrin 1 | 14516.6 | 10700 | 10060.61 |
| Q12962 | P19cd2 | TAF 10 RNA polymerase II; TATA | 5937.255 | 5080 | 3962.323 |
| Q15763 | B20cd2 | t-complex-associated-testis-expr [symbol] | 2022.997 | 2890 | 2885.017 |
| P10599 | B23cd4 | thioredoxin | 11525.16 | 11900 | 6440.37 |
| P13472 | O02ef7 | thymosin; beta 10 | 46982.91 | 74300 | 54263.48 |
| P01253 | L12gh1 | thymosin; beta 4; X chromosome | 23145.54 | 26000 | 16231.51 |
| Q01085 | C02cd2 | TIA1 cytotoxic granule-associate | 2250.456 | 1770 | 2917.342 |
| Q15370 | J12cd2 | transcription elongation factor B [symbol] | 2616.595 | 3980 | 3919.327 |
| O43680 | O11cd2 | transcription factor 21 | 1789.295 | 1790 | 3044.118 |
| P21980 | O16cd2 | transglutaminase 2 (C polypeptid | 3131.079 | 3490 | 1857.596 |
| P29401 | N15cd1 | transketolase (Wernicke-Korsak [symbol] | 3258.575 | 2040 | 2715.206 |
| O60739 | O01cd6 | translation factor sui 1 homolog | 2671.935 | 3400 | 3868.727 |
| Q9NS69 | I02gh7 | translocase of outer mitochondri [symbol] | 2988.423 | 2450 | 3754.914 |
| P00938 | M24cd1 | triosephosphate isomerase 1 | 5701.29 | 5820 | 8345.738 |
| P07226 | G10cd2 | tropomyosin 4 | 2636.256 | 2300 | 3755.882 |
| P04687 | F09cd4 | tubulin; alpha 3 | 20220.03 | 14500 | 22303.88 |
| P04687 | A01cd1 | tubulin; alpha; ubiquitous | 15258.81 | 7840 | 22478.65 |
| Q13509 | E04cd6 | tubulin; beta; 4 | 2338.877 | 2990 | 1853.273 |
| P04350 | F01gh6 | tubulin; beta; 5 | 7573.765 | 4430 | 3448.455 |
| O75347 | O08cd2 | tubulin-specific chaperone a | 3000.065 | 3900 | 3275.629 |
| O75509 | L10ef3 | tumor necrosis factor receptor su | 2245.338 | 4890 | 1881.231 |
| P13693 | G16cd2 | tumor protein; translationally-con | 39878.18 | 60800 | 48468.25 |
| P14625 | G24cd2 | tumor rejection antigen (gp96) 1 | 2185.187 | 2060 | 4905.342 |
| P42655 | K04cd4 | tyrosine 3-monooxygenase/trypt [symbol] | 6584.565 | 5730 | 5558.974 |
| Q04917 | G06cd3 | tyrosine 3-monooxygenase/trypt [symbol] | 8621.322 | 8150 | 4562.366 |
| P29312 | A01cd4 | tyrosine 3-monooxygenase/trypt [symbol] | 5873.282 | 4670 | 2279.148 |
| P29312 | P12cd4 | tyrosine 3-monooxygenase/trypt [symbol] | 4562.964 | 1140 | 1520.2 |
| O14957 | G17cd7 | ubiquinol-cytochrome c reductas | 2728.765 | 3990 | 3332.422 |
| P14793 | D01cd4 | ubiquitin A-52 residue ribosomal | 3536.225 | 1730 | 3258.439 |
| P02248 | D22gh1 | ubiquitin B | 2753.942 | 4430 | 4152.235 |
| O76069 | B12cd4 | ubiquitin-conjugating enzyme E2 | 2001.262 | 1980 | 2223.574 |
| Q9BZL1 | B17gh7 | ubiquitin-like 5 | 4339.474 | 9100 | 5279.835 |
| P08670 | C24cd3 | vimentin | 5940.876 | 8720 | 17783.4 |
| P45880 | C18cd3 | voltage-dependent anion channe | 7005.537 | 7280 | 4758.179 |
| Q9Y277 | C04cd4 | voltage-dependent anion channe | 1863.424 | 2210 | 2573.885 |
| P17861 | O15cd4 | X-box binding protein 1 | 3398.455 | 1600 | 2040.224 |
| O43670 | M04cd3 | zinc finger protein 207 | 3338.117 | 3170 | 3176.631 |
| Q07352 | N21ef6 | zinc finger protein 36; C3H type-I | 6097.384 | 5750 | 4958.224 |
| Q15942 | M10cd3 | zyxin | 2570.21 | 2070 | 2519.748 |
| Q16659 | B17ef5 | mitogen-activated protein kinase | 144.6425 | 141 | 100.0111 |
| O75324 | B05cd3 | stannin | 131.7242 | 172 | 100.0655 |
| Q9BYP8 | E16gh8 | keratin associated protein 17.1 | 176.4958 | 174 | 100.1011 |
| P55291 | J01ab4 | cadherin 15; M-cadherin (myotut | 158.7215 | 128 | 100.3297 |
| P49768 | L14ab7 | presenilin 1 (Alzheimer disease [symbol] | 127.9653 | 124 | 100.3504 |
| P55107 | G10ab5 | growth differentiation factor 10 | 114.7206 | 168 | 100.6326 |
| P17082 | P18cd7 | related RAS viral (r-ras) oncoger | 433.219 | 176 | 100.8465 |
| P23634 | N08ab2 | ATPase; Ca++ transporting; plas | 179.3836 | 276 | 100.9041 |
| P78347 | J20ab5 | general transcription factor II; [symbol] | 151.6834 | 103 | 100.9514 |
| P09912 | F08ef7 | interferon; alpha-inducible protein | 183.6419 | 1050 | 101.0749 |
| Q9H2F5 | F13gh7 | enhancer of polycomb 1 | 127.3938 | 115 | 101.3763 |
| O14811 | M06cd7 | programmed cell death 10 | 152.3457 | 132 | 101.596 |
| O60942 | A17cd4 | RNA guanylyltransferase and 5′- [symbol] | 176.7836 | 97.1 | 101.6765 |
| Q9Y5Y3 | M18cd7 | G protein-coupled receptor 45 | 174.3335 | 161 | 101.8478 |
| P11908 | H15ab8 | phosphoribosyl pyrophosphate s [symbol] | 166.1373 | 174 | 101.9334 |
| Q92851 | P18ab3 | caspase 10; apoptosis-related cy [symbol] | 135.919 | 107 | 101.9435 |
| Q9BQE5 | M03gh7 | apolipoprotein L; 2 | 109.8196 | 148 | 102.0533 |
| P55327 | D05cd2 | tumor protein D52 | 136.2596 | 112 | 102.1111 |
| P28332 | K03ab2 | alcohol dehydrogenase 6 (class [symbol] | 142.4055 | 97.7 | 102.1575 |
| O95861 | E02cd6 | 3′(2′); 5′-bisphosphate nucleotid [symbol] | 128.1251 | 116 | 102.1715 |
| Q9NXJ5 | A10gh2 | hypothetical protein FLJ20208 | 122.4219 | 151 | 102.3038 |
| Q15642 | K09gh1 | thyroid hormone receptor interac | 120.9158 | 142 | 102.3082 |
| O94997 | O09cd7 | decidual protein induced by prog | 174.3087 | 140 | 102.3137 |
| O95453 | G02ab8 | poly(A)-specific ribonuclease (de | 139.9344 | 105 | 102.3217 |
| Q9H9C5 | F01gh7 | KIAA1453 protein | 135.8299 | 126 | 102.4347 |
| P10451 | B13cd1 | secreted phosphoprotein 1 (oste [symbol] | 190.4604 | 76.7 | 102.5193 |
| P53992 | B03cd5 | SEC24 related gene family; mem | 134.307 | 105 | 102.5437 |
| Q9NQW1 | F22gh6 | secretory pathway component S [symbol] | 111.3643 | 158 | 102.6234 |
| Q01449 | A17gh5 | myosin light chain 2a | 179.1031 | 170 | 103.0725 |
| Q9NX46 | K12gh2 | hypothetical protein FLJ20446 | 109.5374 | 159 | 103.1083 |
| P53609 | A23cd1 | protein geranylgeranyltransferas [symbol] | 140.0296 | 128 | 103.1248 |
| Q15029 | J16cd4 | U5 snRNP-specific protein; 116 [symbol] | 119.3284 | 126 | 103.1503 |
| P50458 | O23cd5 | LIM homeobox protein 2 | 145.905 | 120 | 103.1971 |
| P33764 | B24cd1 | S100 calcium binding protein A3 | 150.5991 | 204 | 103.3783 |
| Q9P289 | G03ef3 | Mst3 and SOK1-related kinase | 133.5225 | 116 | 103.4345 |
| P52429 | P17cd3 | diacylglycerol kinase; epsilon (64 | 128.7238 | 123 | 103.5015 |
| P54578 | H17cd5 | ubiquitin specific protease 14 (tR | 160.7495 | 108 | 103.6864 |
| Q13219 | E24ab8 | pregnancy-associated plasma p [symbol] | 112.9052 | 154 | 103.7948 |
| O60547 | M12ab5 | GDP-mannose 4; 6-dehydratase | 147.2805 | 108 | 103.8004 |
| P23297 | I17ef6 | S100 calcium binding protein A1 | 145.2558 | 126 | 103.9033 |
| Q9H5J8 | M01gh6 | hypothetical protein MGC5306 | 216.091 | 91.7 | 104.0432 |
| Q14493 | I08cd4 | stem-loop (histone) binding prote [symbol] | 126.7755 | 155 | 104.1318 |
| Q9NZU0 | O16cd8 | fibronectin leucine rich transmer [symbol] | 284.9107 | 268 | 104.3033 |
| P80370 | B01ef7 | delta-like 1 homolog (Drosophila | 160.1421 | 80 | 104.328 |
| Q9NYV4 | C23ef3 | CDC2-related protein kinase 7 | 128.5947 | 107 | 104.3521 |
| P09016 | C23ef5 | homeo box D4 | 173.2254 | 192 | 104.3556 |
| Q13873 | A16ab3 | bone morphogenetic protein rec [symbol] | 155.8113 | 86.8 | 104.4206 |
| O14878 | B22ef4 | inosine triphosphatase (nucleosi [symbol] | 124.8814 | 120 | 104.4238 |
| P52848 | J01ab6 | N-deacetylase/N-sulfotransferas [symbol] | 166.2411 | 84.4 | 104.4555 |
| Q14848 | A05cd5 | TNF receptor-associated factor 4 | 124.2461 | 136 | 104.4604 |
| O00204 | O04cd2 | sulfotransferase family; cytosolic | 185.8106 | 117 | 104.4824 |
| P35610 | E13gh1 | sterol O-acyltransferase (acyl-Cc | 143.8417 | 88 | 104.5518 |
| Q04760 | L03ab5 | glyoxalase I | 132.6871 | 107 | 104.5782 |
| P51690 | H06ab2 | arylsulfatase E (chondrodysplasi | 177.023 | 76.2 | 104.5865 |
| P26012 | C11ef7 | integrin; beta 8 | 110.7396 | 170 | 104.6296 |
| Q14978 | K11cd5 | nucleolar and coiled-body phosp | 117.0048 | 131 | 104.685 |
| O00764 | D16cd3 | pyridoxal (pyridoxine; vitamin B6 | 160.2678 | 140 | 104.6915 |
| Q9Y6Q5 | P13cd5 | adaptor-related protein complex | 149.3929 | 107 | 104.7705 |
| O95350 | G04cd5 | Homer; neuronal immediate earl [symbol] | 137.2833 | 126 | 104.7829 |
| P49748 | C13ab2 | acyl-Coenzyme A dehydrogenas [symbol] | 121.151 | 141 | 104.7897 |
| P08100 | O24cd1 | rhodopsin (opsin 2; rod pigment) | 125.3282 | 116 | 104.8101 |
| Q15477 | F24cd2 | superkiller viralicidic activity 2-lik [symbol] | 102.5551 | 186 | 104.8138 |
| O43520 | I17cd1 | ATPase; Class I; type 8B; memb [symbol] | 199.1157 | 91.5 | 104.8252 |
| Q13039 | P02ef4 | ATP-binding cassette; sub-family | 175.7841 | 80.5 | 104.8585 |
| O15291 | L12cd1 | solute carrier family 7 (cationic a [symbol] | 276.3689 | 176 | 104.904 |
| Q9NR71 | L23gh4 | mitochondrial ceramidase | 108.9644 | 152 | 104.9361 |
| O60884 | H10cd4 | DnaJ (Hsp40) homolog; subfamil [symbol] | 221.9835 | 239 | 104.9441 |
| Q9BYE0 | J17gh8 | hairy and enhancer of split 7 (Dr [symbol] | 128.0255 | 108 | 104.9542 |
| Q9Y272 | L01ef2 | RAS; dexamethasone-induced 1 | 101.2051 | 166 | 104.9825 |
| Q9BUR5 | M13gh6 | hyphothetical protein MGC4825 | 78.2636 | 239 | 105.0137 |
| O60859 | A13cd7 | neuropathy target esterase | 123.9707 | 117 | 105.029 |
| Q9GZU1 | A20gh4 | mucolipin 1 | 85.32301 | 243 | 105.1039 |
| P19878 | B15ab6 | neutrophil cytosolic factor 2 (65k [symbol] | 310.5103 | 515 | 105.1041 |
| Q13277 | K12cd2 | syntaxin 3A | 118.5375 | 185 | 105.2511 |
| | B04gh8 | reserved | 130.849 | 124 | 105.2594 |
| P32456 | N18ab4 | guanylate binding protein 2; inter | 109.3786 | 142 | 105.2611 |
| Q9H4M9 | E01cd7 | EH-domain containing 1 | 149.0051 | 151 | 105.3893 |
| Q9BTX7 | O19gh6 | chromosome 20 open reading fr [symbol] | 115.9099 | 157 | 105.4128 |
| Q9NVN2 | M02gh3 | solute carrier family 4 (anion exc [symbol] | 123.8178 | 114 | 105.466 |
| Q99758 | A17ab2 | ATP-binding cassette; sub-family | 238.456 | 145 | 105.5504 |
| P43694 | B06ef6 | GATA binding protein 4 | 107.7539 | 161 | 105.5891 |
| Q13825 | C07ab3 | AU RNA binding protein/enoyl-C [symbol] | 123.4494 | 140 | 105.6272 |
| Q92550 | L23ef3 | ring finger protein 10 | 197.4603 | 112 | 105.7052 |
| Q9UJS0 | C12ef3 | solute carrier family 25; member | 119.1806 | 139 | 105.8216 |
| Q99611 | O05ef3 | seleriophosphate synthetase 2 | 168.8742 | 128 | 105.8955 |
| P17275 | F08ab6 | jun B proto-oncogene | 135.9478 | 94.9 | 105.9141 |
| Q13477 | B09cd4 | mucosal vascular addressin cell | 127.8352 | 110 | 105.9239 |
| Q14692 | D09gh1 | KIAA0187 gene product | 114.5267 | 131 | 105.9487 |
| P49908 | D13cd2 | selenoprotein P; plasma; 1 | 140.6083 | 104 | 105.9794 |
| P54803 | M24ab3 | galactosylceramidase (Krabbe di [symbol] | 130.3854 | 105 | 105.9866 |
| P50991 | H09cd6 | chaperonin containing TCP1; su [symbol] | 161.4396 | 148 | 106.0337 |
| P35548 | K11ab7 | msh homeo box homolog 2 (Dro [symbol] | 182.4309 | 124 | 106.057 |
| P11309 | M14ab8 | pim-1 oncogene | 136.3951 | 99 | 106.0757 |
| O75570 | A03cd5 | mitochondrial translational relea [symbol] | 128.9238 | 118 | 106.1208 |
| Q9NY61 | O01ef3 | apoptosis antagonizing transcrip [symbol] | 120.3605 | 124 | 106.1404 |
| Q9BTY8 | D01gh7 | hypothetical protein MGC4342 | 139.5304 | 92.9 | 106.2168 |
| Q9NWS2 | F09gh2 | hypothetical protein FLJ20640 | 183.0823 | 140 | 106.2384 |
| O00180 | H06ab6 | potassium channel; subfamily K; | 129.4915 | 122 | 106.2703 |
| O15382 | I05ab3 | branched chain aminotransferas [symbol] | 136.8391 | 95.5 | 106.3058 |
| Q9Y2Z1 | D05ef2 | CGI-01 protein | 156.4563 | 196 | 106.3369 |
| Q9Y315 | E18ef2 | CGI-26 protein | 135.7793 | 111 | 106.6662 |
| Q9Y592 | P20ef1 | NY-REN-58 antigen | 118.6541 | 121 | 106.7049 |
| Q9NYT0 | D20ef3 | pleckstrin 2 (mouse) homolog | 114.999 | 153 | 106.7508 |
| Q9Y343 | M10ef4 | SBBI31 protein | 123.0625 | 120 | 106.8362 |
| Q9BVI4 | G09gh6 | hypothetical protein MGC3162 | 140.5988 | 123 | 106.8523 |
| P30622 | B20cd1 | restin (Reed-Steinberg cell-expr [symbol] | 116.8074 | 136 | 107.0013 |
| Q08345 | F08ef5 | discoidin domain receptor family; | 266.6468 | 264 | 107.136 |
| P33176 | C16ab7 | kinesin family member 5B | 123.5679 | 113 | 107.2314 |
| P55273 | D14ab4 | cyclin-dependent kinase inhibitor | 99.64375 | 174 | 107.4234 |
| Q9NW92 | E10gh3 | hypothetical protein FLJ10209 | 177.1237 | 168 | 107.454 |
| Q9NZC3 | K03ef3 | membrane interacting protein of [symbol] | 105.8303 | 152 | 107.465 |
| P15172 | M19ab7 | myogenic factor 3 | 132.7839 | 110 | 107.4694 |
| P27144 | A06ab2 | adenylate kinase 3 | 118.5733 | 124 | 107.5998 |
| O60508 | A06ef2 | pre-mRNA splicing factor 17 | 150.1968 | 130 | 107.6187 |
| Q14012 | B08cd3 | calcium/calmodulin-dependent p [symbol] | 113.9231 | 135 | 107.6204 |
| P98179 | G12cd1 | RNA binding motif protein 3 | 140.1356 | 93.2 | 107.7553 |
| Q13223 | E10ab5 | BRF1 homolog; subunit of RNA [symbol] | 150.0469 | 115 | 107.7567 |
| P55198 | N09ab7 | myeloid/lymphoid or mixed-linea [symbol] | 180.7113 | 122 | 107.7762 |
| P46459 | G02ef1 | N-ethylmaleimide-sensitive facto [symbol] | 138.9363 | 95.7 | 107.8044 |
| P34969 | I21ef1 | 5-hydroxytryptamine (serotonin) [symbol] | 218.7666 | 181 | 107.8181 |
| Q14667 | K02gh1 | KIAA0100 gene product | 105.9471 | 222 | 107.8612 |
| Q92830 | J14cd8 | GCN5 general control of amino- [symbol] | 129.4527 | 106 | 107.863 |
| | P14cd2 | RNA binding motif protein; Y chr [symbol] | 112.3917 | 153 | 107.9125 |
| O43252 | J01cd5 | 3′-phosphoadenosine 5′-phosph [symbol] | 194.4946 | 188 | 107.9154 |
| Q9NS93 | G09ef3 | seven transmembrane protein T [symbol] | 138.0932 | 237 | 107.9483 |
| O60616 | E20ab6 | core-binding factor; runt domain; | 489.0924 | 48.1 | 107.9969 |
| P02261 | D19cd4 | H2A histone family; member C | 145.0821 | 84.1 | 108.023 |
| Q14701 | F18ef7 | DNA cross-link repair 1A (PSO2 | 119.2486 | 307 | 108.208 |
| Q9P025 | A04ef8 | HSPC135 protein | 160.3109 | 135 | 108.2493 |
| P06702 | O12ef7 | S100 calcium binding protein A9 | 144.7319 | 163 | 108.2576 |
| Q9Y5Q9 | P05cd7 | general transcription factor IIIC; [symbol] | 138.644 | 220 | 108.3524 |
| P19075 | O20cd2 | transmembrane 4 superfamily m [symbol] | 129.5468 | 103 | 108.6355 |
| O60905 | B16cd4 | sperm associated antigen 9 | 110.7683 | 139 | 108.6913 |
| Q13322 | F24ef5 | growth factor receptor-bound pro [symbol] | 120.8593 | 115 | 108.7182 |
| Q9UQ09 | L01gh1 | KIAA0470 gene product | 190.3667 | 103 | 108.9005 |
| P57053 | J20gh6 | H2B histone family; member S | 113.6057 | 199 | 108.9238 |
| Q92876 | J03ab8 | kallikrein 6 (neurosin; zyme) | 197.2979 | 130 | 108.9308 |
| P09960 | F21ab6 | leukotriene A4 hydrolase | 96.36818 | 185 | 109.0526 |
| Q9HBH1 | O02gh5 | peptide deformylase-like protein | 138.0653 | 91 | 109.0612 |
| P55082 | L15ab7 | microfibrillar-associated protein [symbol] | 121.4467 | 124 | 109.134 |
| Q9NVH6 | B01gh3 | trimethyllysine hydroxylase; epsil [symbol] | 174.7559 | 81.6 | 109.1442 |
| O95249 | K10cd5 | golgi SNAP receptor complex m [symbol] | 107.5878 | 143 | 109.1657 |
| Q13891 | M15ab6 | basic transcription factor 3; like 2 | 126.6543 | 120 | 109.1942 |
| Q9BZM5 | H05gh7 | UL16 binding protein 2 | 133.6731 | 245 | 109.2193 |
| O94913 | O20ef2 | PCF11p homolog | 205.6752 | 80.3 | 109.2409 |
| Q12987 | E03ef4 | acidic 82 kDa protein mRNA | 132.1136 | 117 | 109.4318 |
| Q92499 | D05ab5 | DEAD/H (Asp-Glu-Ala-Asp/His) [symbol] | 176.6983 | 124 | 109.4356 |
| Q13686 | B19ab2 | alkylation repair; alkB homolog | 105.5169 | 143 | 109.4797 |
| P43250 | I08ab4 | G protein-coupled receptor kinas [symbol] | 120.3579 | 113 | 109.4844 |
| Q9UMX0 | L03ef4 | ubiquilin 1 | 122.0111 | 141 | 109.5522 |
| O94905 | G12cd7 | chromosome 8 open reading fra [symbol] | 127.8659 | 107 | 109.6202 |
| Q9UHK0 | K01cd8 | nuclear fragile X mental retardat [symbol] | 157.4757 | 87.6 | 109.6296 |
| P25189 | D01ab6 | myelin protein zero (Charcot-Ma [symbol] | 110.5502 | 132 | 109.6859 |
| Q10567 | M11ab2 | adaptor-related protein complex | 208.9283 | 187 | 109.8204 |
| O15230 | D18cd8 | laminin; alpha 5 | 111.864 | 141 | 109.8469 |
| P52294 | J10ab6 | karyopherin alpha 1 (importin alc [symbol] | 178.7513 | 169 | 109.9831 |
| O60668 | M03cd2 | TAF2 RNA polymerase II; TATA | 123.6244 | 111 | 110.0571 |
| P11688 | J02cd1 | surfactant; pulmonary-associate [symbol] | 136.0267 | 117 | 110.0847 |
| Q9NWB6 | P03gh2 | hypothetical protein FLJ10154 | 174.7789 | 75 | 110.1306 |
| Q9H028 | E22gh8 | histone deacetylase 10 | 107.5034 | 138 | 110.1585 |
| Q9UMZ2 | O22cd7 | AP1 gamma subunit binding prot [symbol] | 120.2821 | 119 | 110.2219 |
| P24752 | C15ab2 | acetyl-Coenzyme A acetyltransfe [symbol] | 216.1409 | 114 | 110.2562 |
| P11230 | B06ab3 | cholinergic receptor; nicotinic; b [symbol] | 106.1774 | 151 | 110.2999 |
| O14977 | O18ef2 | omithine decarboxylase antizym [symbol] | 119.9172 | 129 | 110.3 |
| | E15ef5 | T cell receptor beta locus | 139.7091 | 844 | 110.3287 |
| P56937 | N06ef2 | hydroxysteroid (17-beta) dehydr [symbol] | 227.1137 | 74.5 | 110.3428 |
| O75299 | B21ab7 | kinesin family member C3 | 113.15 | 128 | 110.3535 |
| P16219 | C09ab2 | acyl-Coenzyme A dehydrogenas [symbol] | 109.4688 | 136 | 110.3636 |
| Q9NZ48 | J20gh3 | uncharacterized hematopoietic s [symbol] | 112.5665 | 189 | 110.3995 |
| Q9P163 | L10gh4 | hypothetical protein PRO2521 | 101.1317 | 159 | 110.4184 |
| Q9BVT8 | I07gh8 | hypothetical protein MGC5442 | 135.5302 | 137 | 110.5044 |
| Q99933 | E02ef6 | BCL2-associated athanogene | 119.7332 | 115 | 110.533 |
| O14645 | M12cd3 | dynein; axonemal; light intermed [symbol] | 164.1806 | 207 | 110.5493 |
| Q9BRR9 | E01gh8 | Rho GTPase activating protein 9 | 120.3646 | 115 | 110.5606 |
| Q9Y3E7 | J19ef2 | CGI-149 protein | 158.4397 | 202 | 110.6074 |
| P46976 | C09ab6 | glycogenin | 127.6502 | 117 | 110.6815 |
| O75928 | C01cd5 | Protein inhibitor of activated STA | 98.84393 | 173 | 110.7286 |
| Q9NX63 | I18gh2 | hypothetical protein FLJ20420 | 123.3725 | 159 | 110.8068 |
| Q9UBS0 | I20cd2 | ribosomal protein S6 kinase; 70k | 186.953 | 155 | 110.8188 |
| Q15185 | F18cd6 | unactive progesterone receptor; [symbol] | 169.4269 | 72.3 | 110.8534 |
| O60516 | J18cd3 | eukaryotic translation initiation fa | 104.3739 | 147 | 110.9085 |
| Q16651 | J01ab8 | protease; serine; B (prostasin) | 334.0498 | 125 | 110.9253 |
| P48788 | E22cd2 | troponin I; skeletal; fast | 121.3602 | 110 | 110.9357 |
| Q01968 | N06ab7 | oculocerebrorenal syndrome of L | 169.0079 | 95.9 | 110.9507 |
| Q9H6Z6 | L11gh7 | hypothetical protein FLJ21628 | 115.1546 | 126 | 111.0337 |
| Q9NQ55 | B01gh4 | peter pan homolog (Drosophila) | 122.449 | 120 | 111.0627 |
| O95359 | A24gh1 | transforming; acidic coiled-coil α | 108.2194 | 139 | 111.0999 |
| P01189 | I15ab8 | proopiomelanocortin (adrenocort | 145.0634 | 90.4 | 111.1389 |
| Q9Y525 | G13cd8 | v-maf musculoaponeurotic fibros [symbol] | 119.023 | 141 | 111.1934 |
| Q9UHC9 | L09cd8 | NPC1 (Niemann-Pick disease; ty | 107.7403 | 138 | 111.3741 |
| Q9HBT2 | P14gh7 | hypothetical protein PP1057 | 147.5097 | 98 | 111.4385 |
| O00219 | M22ef1 | hyaluronan synthase 3 | 224.5087 | 89 | 111.4539 |
| P22735 | M16cd1 | transglutaminase 1 (K polypeptid | 136.8808 | 91 | 111.5589 |
| O15388 | A11ab4 | sialic acid binding Ig-like lectin 6 | 105.2847 | 147 | 111.6016 |
| O95389 | J01cd4 | WNT1 inducible signaling pathw [symbol] | 134.8825 | 94 | 111.6123 |
| Q06190 | D21ab8 | protein phosphatase 2 (formerly: | 134.6973 | 102 | 111.6192 |
| P50226 | C03cd3 | sulfotransferase family; cytosolic | 147.6874 | 101 | 111.8328 |
| Q16632 | N10ab5 | general transcription factor IIIC; [symbol] | 120.7982 | 116 | 111.85 |
| P54762 | A24ef7 | EphB1 | 107.3397 | 168 | 111.8669 |
| Q15269 | E15cd1 | PWP2 periodic tryptophan protei | 125.0688 | 107 | 111.8713 |
| O14495 | H10cd3 | phosphatidic acid phosphatase [symbol] | 120.761 | 109 | 111.9087 |
| Q9P0U0 | N24ef3 | PC326 protein | 152.5639 | 101 | 111.9543 |
| P50120 | J02ab8 | retinol binding protein 2; cellular | 231.806 | 217 | 111.9639 |
| Q9UHR0 | D08gh1 | G protein-coupled receptor kinas | 111.83 | 151 | 111.9778 |
| Q13977 | A12ef1 | cerebellar degeneration-related [symbol] | 105.2052 | 180 | 112.0477 |
| P51692 | C18cd8 | signal transducer and activator o | 126.7779 | 100 | 112.0645 |
| P51164 | N12ab2 | ATPase; H+/K+ exchanging; bet [symbol] | 136.4695 | 112 | 112.2007 |
| Q9NXH3 | C02gh2 | protein phosphatase 1; regulator | 113.3081 | 127 | 112.2521 |
| | C04gh7 | ER to nucleus signalling 2 | 112.9203 | 125 | 112.2701 |
| P16435 | G16ef6 | P450 (cytochrome) oxidoreducta [symbol] | 110.2003 | 132 | 112.2899 |
| | E05gh6 | hypothetical protein MGC5338 | 116.1511 | 137 | 112.3522 |
| Q02080 | L03ab7 | MADS box transcription enhance | 138.6124 | 100 | 112.3745 |
| Q15334 | J06ab7 | lethal giant larvae homolog 1 (Dr | 123.3076 | 110 | 112.3893 |
| P10636 | J13ab7 | microtubule-associated protein [symbol] | 115.8818 | 142 | 112.4092 |
| Q9Y4J6 | A01gh2 | zinc finger protein 6 (CMPX1) | 138.8767 | 102 | 112.4376 |
| Q9UHY7 | P18gh4 | E-1 enzyme | 108.9217 | 134 | 112.5485 |
| Q14147 | K04gh1 | DEAD/H (Asp-Glu-Ala-Asp-/His) [symbol] | 117.5358 | 119 | 112.7866 |
| O95880 | N06gh4 | KIAA1513 protein | 168.2097 | 104 | 112.9115 |
| Q15013 | J23ef3 | gene predicted from cDNA with a | 104.556 | 198 | 113.0807 |
| Q9P1E2 | E01gh4 | hypothetical protein PRO2219 | 138.6039 | 107 | 113.1066 |
| P07203 | H23ef1 | glutathione peroxidase 1 | 120.5127 | 114 | 113.1788 |
| Q13206 | M07ab5 | DEAD/H (Asp-Glu-Ala-Asp/His) [symbol] | 140.5274 | 92.5 | 113.1804 |
| O15085 | L15ef3 | Rho guanine nucleotide exchang | 121.8405 | 111 | 113.2492 |
| P48426 | C07cd1 | phosphatidylinositol-4-phosphate | 232.9191 | 163 | 113.2533 |
| Q9NRQ2 | K20gh4 | phospholipid scramblase 4 | 126.2397 | 116 | 113.2849 |
| O43171 | B24cd3 | CDC14 cell division cycle 14 hon | 126.784 | 113 | 113.382 |
| Q9HB90 | M02gh5 | Rag C protein | 132.9037 | 136 | 113.4299 |
| Q9H733 | I21gh5 | single Ig IL-1R-related molecule | 113.5449 | 137 | 113.4753 |
| P43353 | C06ab2 | aldehyde dehydrogenase 3 famil | 141.7651 | 101 | 113.5244 |
| Q15053 | G14gh1 | KIAA0040 gene product | 111.1705 | 146 | 113.5775 |
| P22301 | H01ef7 | interleukin 10 | 174.8595 | 108 | 113.5877 |
| Q9BW47 | N12gh5 | chromosome 20 open reading fr [symbol] | 125.8289 | 154 | 113.6231 |
| P21917 | M03ab6 | dopamine receptor D4 | 158.6974 | 151 | 113.7899 |
| Q9Y6K5 | M19cd1 | 2′-5′-oligoadenylate synthetase 3 | 127.2648 | 142 | 113.9993 |
| Q9Y2A4 | N22cd5 | Kruppel-type zinc finger (C2H2) | 136.0849 | 133 | 114.1396 |
| Q16880 | A16cd3 | UDP glycosyltransferase 8 (UDP | 112.9309 | 133 | 114.1612 |
| O95747 | D09cd5 | oxidative-stress responsive 1 | 113.39 | 123 | 114.2384 |
| P25106 | L12ef4 | G protein-coupled receptor | 100.7123 | 147 | 114.2482 |
| | H23ab6 | immunoglobulin superfamily; me [symbol] | 102.6971 | 145 | 114.3219 |
| P45984 | L18ef5 | mitogen-activated protein kinase | 139.4168 | 88 | 114.3405 |
| Q9NPF7 | I03ef3 | interleukin 23; alpha subunit p19 | 98.83593 | 182 | 114.4153 |
| Q9UH94 | L07cd8 | prolactin regulatory element bind | 140.4102 | 106 | 114.4158 |
| Q16559 | F05cd2 | T-cell acute lymphocytic leukem [symbol] | 131.4974 | 111 | 114.5482 |
| Q9UGH0 | C02cd8 | solute carrier family 17 (anion/su | 98.73308 | 158 | 114.6616 |
| Q15544 | J13cd2 | TAF11 RNA polymerase II; TATA | 117.4634 | 123 | 114.6792 |
| O00628 | N24ab7 | peroxisomal biogenesis factor 7 | 156.16 | 96.9 | 114.7281 |
| O43241 | F07ab5 | golgi autoantigen; golgin subfam | 123.9421 | 104 | 114.8381 |
| Q9Y6N1 | M14ab4 | COX11 homolog; cytochrome c c | 127.1203 | 100 | 114.8993 |
| Q9UHX5 | O01ef4 | HT002 protein; hypertension-rela | 98.26523 | 155 | 114.9242 |
| Q01664 | A02cd2 | transcription factor AP-4 (activati | 268.3453 | 66.7 | 115.0377 |
| Q9BUI1 | O03gh6 | leukocyte receptor cluster (LRC) | 112.2417 | 125 | 115.039 |
| Q9HBL5 | M17gh4 | AD023 protein | 103.9999 | 137 | 115.0443 |
| Q9NXV2 | N08gh2 | hypothetical protein FLJ20040 | 126.6647 | 147 | 115.1386 |
| Q15424 | D08cd1 | scaffold attachment factor B | 134.9613 | 92.7 | 115.1845 |
| P26440 | F06ab6 | isovaleryl Coenzyme A dehydrog | 178.7093 | 115 | 115.2412 |
| O60264 | J11cd3 | SWI/SNF related; matrix associa | 105.7775 | 132 | 115.2498 |
| Q15286 | K07cd7 | RAB35; member RAS oncogene | 138.2766 | 183 | 115.3443 |
| Q9UGK0 | I10ef4 | DIPB protein | 303.2745 | 172 | 115.4096 |
| Q92980 | J06cd5 | putative protein similar to nessy [symbol] | 142.089 | 181 | 115.4166 |
| Q99726 | M06cd3 | solute carrier family 30 (zinc tran | 112.8008 | 121 | 115.4854 |
| P01111 | O17ab8 | neuroblastoma RAS viral (v-ras) | 123.6283 | 108 | 115.5932 |
| Q92574 | G13cd3 | tuberous scierosis 1 | 109.8519 | 128 | 115.7107 |
| P16106 | C01gh7 | H3 histone family; member I | 134.9364 | 118 | 115.868 |
| Q9NWX5 | O24gh2 | hypothetical protein FLJ20548 | 118.1581 | 112 | 115.9919 |
| Q9Y6B7 | M05ab3 | adaptor-related protein complex | 217.8572 | 170 | 116.0173 |
| Q9Y4F1 | J04cd5 | FERM; RhoGEF (ARHGEF) and | 108.6215 | 130 | 116.0342 |
| Q06710 | M14cd3 | paired box gene 8 | 120.1357 | 129 | 116.1006 |
| Q9H6N1 | J17gh5 | hypothetical protein FLJ22056 | 214.4909 | 242 | 116.2509 |
| Q9NSY1 | K09gh3 | homolog of mouse BMP-2 inducil [symbol] | 111.9034 | 123 | 116.2514 |
| Q14106 | H20cd6 | transducer of ERBB2; 2 | 117.9634 | 134 | 116.2536 |
| Q9UK11 | B02ef4 | zinc finger protein 223 | 119.1221 | 133 | 116.2873 |
| P08069 | O17ef6 | insulin-like growth factor 1 recep [symbol] | 106.3434 | 178 | 116.3103 |
| Q9H6F5 | I21gh6 | hypothetical protein MGC2574 | 120.5097 | 112 | 116.3129 |
| Q14373 | O04ab3 | galactose-1-phosphate uridylyltr [symbol] | 157.7476 | 231 | 116.3555 |
| P20393 | G04ef5 | nuclear receptor subfamily 1; gro [symbol] | 107.1138 | 138 | 116.4228 |
| P36915 | O01ab5 | guanine nucleotide binding prote [symbol] | 120.9306 | 111 | 116.4819 |
| O95109 | G02cd8 | chromosome 20 open reading fr [symbol] | 143.5726 | 103 | 116.5321 |
| Q9NVS2 | I24gh3 | mitochondrial ribosomal protein [symbol] | 117.9573 | 124 | 116.6011 |
| Q92950 | B08cd6 | CUG triplet repeat; RNA binding | 109.8876 | 129 | 116.7366 |
| Q14977 | C04ab7 | SP110 nuclear body protein | 107.0969 | 158 | 116.8973 |
| P16106 | O04gh6 | H3 histone family; member A | 94.76745 | 159 | 116.9796 |
| Q9Y3C1 | N18ef2 | hypothetical protein HSPC111 | 145.1935 | 164 | 117.0185 |
| P37287 | C11ef1 | phosphatidylinositol glycan; clas [symbol] | 132.5354 | 109 | 117.2467 |
| Q9UIG5 | G05ef4 | SEEK1 protein | 102.5149 | 148 | 117.279 |
| Q9UNE2 | G15cd6 | rabphilin 3A-like (without C2 dorr | 110.3023 | 123 | 117.2874 |
| Q14123 | A17cd1 | phosphodiesterase 1C; calmodul | 121.3078 | 132 | 117.3754 |
| Q16206 | P03ab2 | cytosolic ovarian carcinoma anti [symbol] | 116.5183 | 157 | 117.4492 |
| Q9NWT1 | D21gh2 | hypothetical protein FLJ20624 | 116.0517 | 116 | 117.4816 |
| Q12756 | C05ab3 | axonal transport of synaptic vesi [symbol] | 138.4398 | 232 | 117.5489 |
| P28288 | B10ab8 | ATP-binding cassette; sub-family | 140.4464 | 95.9 | 117.5959 |
| P05198 | G08ab5 | eukaryotic translation initiation fa [symbol] | 115.5074 | 119 | 117.6033 |
| Q9H0V9 | D08gh7 | hypothetical protein DKFZp564L [symbol] | 128.832 | 101 | 117.6689 |
| Q92984 | M10ef1 | interferon-induced protein 35 | 258.0737 | 221 | 117.8182 |
| O95498 | A13cd5 | vanin 2 | 109.7731 | 123 | 117.8273 |
| Q9H1E1 | J01gh8 | ribonuclease 7 | 109.332 | 131 | 117.8377 |
| Q9NV88 | H01gh3 | hypothetical protein FLJ10871 | 108.5849 | 125 | 117.8411 |
| Q9Y310 | F06cd6 | SnRNP assembly defective 1 ho [symbol] | 119.9305 | 113 | 117.8563 |
| P48382 | O06cd1 | regulatory factor X; 5 (influences | 191.5142 | 122 | 118.0773 |
| P52738 | K04cd3 | zinc finger protein 140 (clone pH | 136.6586 | 154 | 118.1501 |
| Q9NRW1 | G23ef3 | RAB6B; member RAS oncogene | 100.578 | 179 | 118.1659 |
| O15298 | A02ab3 | basic leucine zipper nuclear fact [symbol] | 266.9887 | 110 | 118.2062 |
| Q9NUN5 | D14gh3 | hypothetical protein FLJ11240 | 114.5392 | 116 | 118.2691 |
| Q07699 | L03cd1 | sodium channel; voltage-gated; [symbol] | 116.5431 | 113 | 118.3239 |
| P27701 | F10ab6 | kangai 1 (suppression of tumorig [symbol] | 116.2761 | 108 | 118.3881 |
| Q9UER5 | O02ef3 | TNF-induced protein | 104.9755 | 131 | 118.4653 |
| Q8TAC6 | I24gh7 | chromosome 11 open reading fr [symbol] | 117.4257 | 138 | 118.5887 |
| Q9NRW9 | K16gh4 | angiotensin II; type I receptor-as [symbol] | 136.6357 | 105 | 118.6777 |
| O95147 | O15cd7 | dual specificity phosphatase 14 | 181.9968 | 200 | 118.7007 |
| Q13895 | A19ef7 | bystin-like | 108.0232 | 124 | 118.7967 |
| P31645 | N03ef5 | solute carrier family 6 (neurotran [symbol] | 114.6464 | 189 | 118.8487 |
| P51511 | N17ef7 | matrix metalloproteinase 15 (me [symbol] | 122.9018 | 125 | 118.8609 |
| Q15154 | O11cd1 | pericentriolar material 1 | 127.8378 | 139 | 118.8759 |
| Q00007 | E17ef6 | protein phosphatase 2 (formerly; | 108.364 | 123 | 118.9838 |
| P55822 | L08cd2 | SH3 domain binding glutamic aci [symbol] | 131.7189 | 133 | 119.0263 |
| Q14442 | L20ab8 | phosphatidylinositol glycan; clas [symbol] | 121.0635 | 114 | 119.0306 |
| P54792 | L04ab4 | dishevelled; dsh homolog 1 (Dro [symbol] | 126.677 | 113 | 119.067 |
| P42336 | A03ef6 | phosphoinositide-3-kinase; catal [symbol] | 101.0905 | 143 | 119.1581 |
| Q9UMQ3 | G15ab3 | BarH-like homeobox 2 | 99.5713 | 179 | 119.1741 |
| Q9UPY5 | M04ef3 | solute carrier family 7; (cationic [symbol] | 173.9407 | 69.5 | 119.3079 |
| P19113 | H01ef6 | histidine decarboxylase | 111.9277 | 119 | 119.3155 |
| O95394 | B16ef3 | N-acetylglucosamine-phosphate | 124.8164 | 112 | 119.3184 |
| P22557 | A14ab2 | aminolevulinate; delta-; synthase | 202.1179 | 129 | 119.3857 |
| Q9Y3A2 | G10ef2 | CGI-94 protein | 108.7497 | 167 | 119.4751 |
| Q9H5J4 | I05gh6 | long-chain fatty-acyl elongase | 135.0119 | 87 | 119.5181 |
| P16083 | E15ab8 | NAD(P)H dehydrogenase; quino [symbol] | 154.7986 | 98.6 | 119.5724 |
| Q99819 | D02ab2 | Rho GDP dissociation inhibitor ( [symbol] | 125.7962 | 106 | 119.5853 |
| O75569 | F06cd3 | protein kinase; interferon-inducib [symbol] | 100.3086 | 137 | 119.6257 |
| O15038 | F12ef7 | serine/arginine repetitive matrix [symbol] | 125.19 | 96.6 | 119.719 |
| Q9HBH5 | K09gh4 | PAN2 protein | 102.0601 | 139 | 119.7321 |
| P41231 | C22ab8 | purinergic receptor P2Y; G-prote [symbol] | 131.9219 | 130 | 119.8105 |
| O15397 | D01cd6 | RAN binding protein 8 | 114.6925 | 118 | 119.9197 |
| Q9UDY8 | C17cd7 | mucosa associated lymphoid tiss [symbol] | 114.4769 | 120 | 119.9403 |
| O95819 | E22cd5 | mitogen-activated protein kinase | 92.42403 | 156 | 119.956 |
| Q12999 | J01ef5 | sarcoma amplified sequence | 114.9397 | 110 | 119.9579 |
| P25440 | D07cd2 | bromodomain containing 2 | 116.6707 | 108 | 120.0152 |
| P20827 | H21ef7 | ephrin-A1 | 298.0458 | 760 | 120.3333 |
| Q9H652 | P20gh8 | hypothetical protein MGC4171 | 129.5733 | 107 | 120.4428 |
| P31483 | L20ef4 | TIA1 cytotoxic granule-associate | 105.4425 | 129 | 120.5386 |
| Q9BSM8 | G15gh8 | hypothetical protein MGC10812 | 148.2279 | 273 | 120.573 |
| P36897 | F04ef5 | transforming growth factor; beta [symbol] | 115.7567 | 108 | 120.6724 |
| Q9UMR9 | E08ef3 | YME1-like 1 (S. cerevisiae) | 256.0314 | 197 | 120.7227 |
| Q92560 | G11ab3 | BRCA1 associated protein-1 (ubi [symbol] | 101.36 | 161 | 120.7575 |
| Q9NWU1 | D05gh2 | hypothetical protein FLJ20604 | 99.76081 | 154 | 120.7609 |
| Q92696 | N14ab8 | Rab geranylgeranyltransferase; [symbol] | 222.7157 | 272 | 120.7706 |
| O95236 | M24ef3 | apolipoprotein L; 3 | 106.592 | 130 | 120.798 |
| Q13202 | E21ab5 | dual specificity phosphatase 8 | 146.3963 | 129 | 120.7987 |
| P11277 | M02cd1 | spectrin; beta; erythrocytic (inclu [symbol] | 112.72 | 114 | 120.8055 |
| O95397 | P18ef1 | putative glycolipid transfer protei [symbol] | 99.78536 | 143 | 120.8708 |
| P05423 | A20ab3 | BN51 (BHK21) temperature sens | 131.8122 | 95.5 | 120.9201 |
| Q14338 | I17ab6 | fucosyltransferase 2 (secretor st [symbol] | 99.71915 | 181 | 120.9483 |
| Q9BYI8 | A22gh8 | AIE-75 binding protein protein | 90.11911 | 164 | 121.0209 |
| Q06203 | D01ab8 | phosphoribosyl pyrophosphate a | 216.3154 | 105 | 121.029 |
| Q9NZ33 | P10gh4 | brain expressed; X-linked 1 | 113.8613 | 162 | 121.0508 |
| P04196 | O10ab6 | histidine-rich glycoprotein | 116.1305 | 116 | 121.1163 |
| O96029 | F03cd7 | opioid growth factor receptor | 201.3579 | 93.5 | 121.2888 |
| Q01196 | I16ab5 | runt-related transcription factor 1 | 441.1217 | 136 | 121.3316 |
| Q9UHL4 | J17cd8 | dipeptidylpeptidase 7 | 180.3445 | 191 | 121.351 |
| P55211 | P16ab3 | caspase 9; apoptosis-related cys | 113.5416 | 111 | 121.3742 |
| P21283 | A07ab3 | ATPase; H+ transporting; lysoso [symbol] | 288.6034 | 288 | 121.3828 |
| O43422 | P02ab8 | protein-kinase; interferon-inducit [symbol] | 131.8635 | 100 | 121.394 |
| P78406 | L01cd3 | RAE1 RNA export 1 homolog (S. | 147.1192 | 97.1 | 121.4625 |
| P43362 | J18ab7 | melanoma antigen; family A; 9 | 99.05783 | 139 | 121.5326 |
| Q9H693 | E12gh6 | hypothetical protein FLJ22477 | 171.7699 | 96.4 | 121.6097 |
| Q9UJ83 | A11cd8 | 2-hydroxyphytanoyl-CoA lyase | 99.45467 | 141 | 121.6399 |
| Q05048 | I15ab4 | cleavage stimulation factor; 3′ pr [symbol] | 124.1609 | 105 | 121.6764 |
| P19784 | D11ab4 | casein kinase 2; alpha prime pol [symbol] | 94.68573 | 152 | 121.7819 |
| Q9BUX8 | D06gh6 | homolog of yeast ribosome biog [symbol] | 167.4752 | 178 | 121.8214 |
| Q9Y5P6 | D23cd8 | GDP-mannose pyrophosphoryla [symbol] | 166.4572 | 89.4 | 121.8219 |
| Q9Y3E1 | F16af1 | hepatoma-derived growth factor: | 97.00713 | 148 | 121.9163 |
| P40426 | O07cd1 | pre-B-cell leukemia transcription | 122.6888 | 94.8 | 121.9253 |
| P11217 | K01cd1 | phosphorylase; glycogen; muscl [symbol] | 314.8041 | 110 | 122.008 |
| Q16563 | D18cd2 | synaptophysin-like protein | 97.62823 | 144 | 122.0944 |
| O95698 | L07ab6 | inhibitor of growth family; membe | 102.2388 | 135 | 122.1358 |
| P19447 | K24ef6 | excision repair cross-complemen [symbol] | 95.41189 | 148 | 122.1425 |
| Q9UK39 | D08cd7 | CCR4 carbon catabolite repressi [symbol] | 173.8761 | 262 | 122.1589 |
| Q15785 | E23cd7 | translocase of outer mitochondri [symbol] | 296.8521 | 144 | 122.1776 |
| Q99571 | C14ab8 | purinergic receptor P2X; ligand- [symbol] | 418.1812 | 410 | 122.3053 |
| Q9BTY2 | E24gh8 | hypothetical protein MGC1314 si [symbol] | 131.9449 | 91.5 | 122.3524 |
| Q9P0P1 | D12ef1 | hypothetical protein FLJ10769 | 147.9172 | 191 | 122.4861 |
| Q9Y324 | P01ef1 | CGI-35 protein | 129.1118 | 97 | 122.5531 |
| P37286 | O17cd3 | ubiquitin-conjugating enzyme E2 | 98.88763 | 165 | 122.5669 |
| Q00577 | O07ef5 | purine-rich element binding prot [symbol] | 116.0341 | 123 | 122.5705 |
| O75152 | L21gh1 | KIAA0663 gene product | 101.4464 | 140 | 122.5789 |
| P23610 | H06cd7 | coagulation factor VIII-associate [symbol] | 164.911 | 103 | 122.5902 |
| O75436 | M20cd5 | vacuolar protein sorting 26 (yeas [symbol] | 118.1135 | 108 | 122.6522 |
| Q9HA40 | B23gh5 | hypothetical protein FLJ12287 si [symbol] | 104.6286 | 126 | 122.666 |
| Q9NX62 | I20gh2 | hypothetical protein FLJ20421 | 103.7764 | 136 | 122.701 |
| O76062 | E04cd2 | transmembrane 7 superfamily m [symbol] | 301.1903 | 215 | 122.7645 |
| Q16445 | H02ab3 | gamma-aminobutyric acid (GAB [symbol] | 150.641 | 87.5 | 122.7829 |
| Q08462 | G11ef6 | adenylate cyclase 2 (brain) | 107.541 | 146 | 122.8502 |
| P78560 | A22ef6 | CASP2 and RIPK1 domain conta [symbol] | 108.4508 | 153 | 122.8627 |
| P19634 | N17ef5 | solute carrier family 9 (sodium/h [symbol] | 118.9969 | 99 | 122.898 |
| P28370 | N12cd1 | SWI/SNF related; matrix associa [symbol] | 115.471 | 106 | 122.9184 |
| Q92503 | H06cd1 | SEC14-like 1 (S. cerevisiae) | 151.5939 | 151 | 122.9424 |
| Q9UL02 | H24ef1 | L-kynurenine/alpha-aminoadipat [symbol] | 105.6951 | 124 | 123.0487 |
| Q9Y5T5 | J13cd6 | ubiquitin specific protease 16 | 117.1056 | 120 | 123.0646 |
| | G03gh8 | protocadherin 15 | 153.0978 | 126 | 123.1548 |
| P15848 | H02ab2 | arylsulfatase B | 149.4791 | 95.6 | 123.1651 |
| Q9UP66 | P24ab6 | low density lipoprotein receptor- [symbol] | 114.2435 | 124 | 123.2083 |
| O75160 | P07gh1 | KIAA0672 gene product | 121.9398 | 117 | 123.2554 |
| P78385 | L14ab6 | keratin; hair; basic: 3 | 100.703 | 135 | 123.2569 |
| O94967 | B21ef8 | KIAA0893 protein | 92.15324 | 158 | 123.2707 |
| O95719 | K01cd5 | xenotropic and polytropic retrovir [symbol] | 108.0877 | 122 | 123.3497 |
| P48147 | F09ab8 | prolyl endopeptidase | 130.0641 | 129 | 123.3564 |
| P20231 | B15gh7 | tryptase beta 2 | 106.6865 | 130 | 123.4905 |
| O95299 | G02ab7 | NADH dehydrogenase (ubiquino [symbol] | 107.385 | 129 | 123.5991 |
| O75033 | I18gh1 | KIAA0445 gene product | 121.5196 | 122 | 123.624 |
| P51668 | D03cd4 | ubiquitin-conjugating enzyme E2 | 99.84401 | 137 | 123.637 |
| P28289 | E08cd2 | tropomodulin | 303.877 | 272 | 123.6484 |
| Q15631 | O12ef6 | translin | 173.134 | 152 | 123.6887 |
| P49868 | F03cd2 | sulfotransferase; estrogen-prefer | 122.2586 | 95.1 | 123.7564 |
| P51959 | O12ef5 | cyclin G1 | 116.6841 | 102 | 123.7661 |
| Q9Y5V0 | J16ef1 | HSPC038 protein | 114.0123 | 179 | 123.7928 |
| P56270 | I12ef1 | MYC-associated zinc finger prot [symbol] | 248.3395 | 140 | 123.8618 |
| Q9HD23 | O11gh4 | MRS2-like; magnesium homeost [symbol] | 140.8864 | 117 | 123.8621 |
| P78358 | O03ab6 | cancer/testis antigen 1 | 217.9167 | 137 | 123.9307 |
| O00327 | D24ab2 | aryl hydrocarbon receptor nuclea [symbol] | 118.2008 | 185 | 123.9698 |
| Q9H969 | F08gh5 | hypothetical protein FLJ12969 | 89.23821 | 177 | 124.0248 |
| O15504 | F01cd7 | nucleoporin-like protein 1 | 200.4449 | 124 | 124.2046 |
| P35250 | I24ef8 | replication factor C (activator 1); | 111.3418 | 112 | 124.3534 |
| Q9BXC0 | F17gh8 | G protein-coupled receptor 81 | 104.4833 | 128 | 124.3568 |
| O15232 | A19ef1 | matrilin 3 | 113.1634 | 110 | 124.4317 |
| O76049 | L19cd5 | spectrin SH3 domain binding pro [symbol] | 171.9625 | 299 | 124.5037 |
| Q14145 | C21cd8 | Kelch-like ECH-associated protei [symbol] | 193.9671 | 120 | 124.5928 |
| P20848 | A10cd1 | serine (or cysteine) proteinase in | 97.77909 | 161 | 124.6004 |
| Q9P0N6 | C19ef3 | hypothetical protein HSPC242 | 112.0065 | 109 | 124.6034 |
| P05188 | E12ab2 | alkaline phosphatase; placental [symbol] | 88.50098 | 164 | 124.6125 |
| Q9UBJ4 | A01gh5 | transposon-derived Buster1 tran [symbol] | 131.0923 | 97.3 | 124.7023 |
| Q13876 | I24ef7 | quiescin Q6 | 144.3305 | 109 | 124.7205 |
| Q9UBF2 | F12cd7 | coatomer protein complex; subur | 110.1673 | 115 | 124.9117 |
| P43026 | I23cd3 | growth differentiation factor 5 (ca [symbol] | 101.9864 | 152 | 124.9218 |
| Q9HD20 | E14gh4 | CGI-152 protein | 111.6731 | 134 | 124.9301 |
| Q96KK6 | P04ab8 | RAB2; member RAS oncogene f [symbol] | 119.8171 | 106 | 124.9467 |
| P40617 | D18ab2 | ADP-ribosylation factor-like 4 | 124.1402 | 143 | 125.0573 |
| O75651 | L12gh2 | unc-93 homolog A (C. elegans) | 131.989 | 128 | 125.0667 |
| Q9Y2S1 | L04ef1 | galanin-related peptide | 135.6724 | 134 | 125.067 |
| Q13330 | M20ef5 | metastasis associated 1 | 104.9962 | 118 | 125.1131 |
| Q9Y389 | L22gh4 | aminoadipate-semialdehyde deh [symbol] | 115.3352 | 104 | 125.222 |
| O60513 | I03gh1 | UDP-Gal: betaGlcNAc beta 1; 4- [symbol] | 113.705 | 106 | 125.2504 |
| O00212 | C15ef6 | ras homolog gene family; memb [symbol] | 407.7018 | 492 | 125.2682 |
| Q9NXE1 | J04gh2 | membrane-associated nucleic ac [symbol] | 96.25484 | 146 | 125.3861 |
| O95405 | A16cd5 | MAD; mothers against decapent [symbol] | 113.9303 | 223 | 125.3908 |
| P01112 | K12ab7 | v-Ha-ras Harvey rat sarcoma vir [symbol] | 129.7326 | 104 | 125.3947 |
| O15120 | F11cd6 | 1-acylglycerol-3-phosphate O-ac [symbol] | 264.2959 | 238 | 125.4039 |
| P12645 | N10ab3 | bone morphogenetic protein 3 (o [symbol] | 107.6548 | 117 | 125.4244 |
| P50579 | I01cd7 | methionyl aminopeptidase 2 | 144.5853 | 95.1 | 125.4613 |
| O14908 | K01cd6 | regulator of G-protein signalling | 204.527 | 200 | 125.5241 |
| Q9HCC6 | I17gh4 | bHLH factor Hes4 | 82.07855 | 213 | 125.5621 |
| Q15737 | E23gh1 | suppressor of Ty 6 homolog (S. [symbol] | 112.2771 | 140 | 125.5917 |
| Q9Y519 | A17cd8 | chromosome 22 open reading fra [symbol] | 100.4037 | 131 | 125.6586 |
| Q9Y5J0 | J01ef2 | mitochondrial ribosomal protein [symbol] | 117.4353 | 107 | 125.8555 |
| P51687 | O16cd1 | sulfite oxidase | 105.2314 | 128 | 125.9497 |
| P51178 | A13ef6 | phospholipase C; delta 1 | 126.4221 | 162 | 126.0345 |
| Q9H8J6 | H23gh5 | NDRG family member 3 | 121.7115 | 198 | 126.048 |
| O95900 | B20ef8 | hypothetical protein CLONE2492 | 165.6404 | 257 | 126.0637 |
| Q9Y6N3 | B01cd5 | chloride channel; calcium activat [symbol] | 99.89597 | 135 | 126.1695 |
| Q07869 | C21cd1 | peroxisome proliferative activate [symbol] | 120.1031 | 112 | 126.2127 |
| Q9Y333 | I07gh4 | chromosome 6 open reading frar [symbol] | 109.0761 | 114 | 126.2563 |
| Q9NPD1 | L06gh2 | G protein-coupled receptor 85 | 206.0578 | 285 | 126.2683 |
| Q15928 | K06cd3 | zinc finger protein 141 (clone pH | 101.1559 | 188 | 126.2732 |
| O43826 | I04ab4 | glucose-6-phosphatase; transpo [symbol] | 229.7084 | 111 | 126.3113 |
| Q9NWL0 | J05gh2 | hypothetical protein FLJ20758 | 114.0453 | 120 | 126.3891 |
| P31629 | B04ef6 | human immunodeficiency virus t [symbol] | 116.0174 | 211 | 126.4284 |
| O15530 | I10ab8 | 3-phosphoinositide dependent p [symbol] | 186.1234 | 193 | 126.45 |
| Q9Y2R5 | L21ef4 | mitochondrial ribosomal protein [symbol] | 106.3741 | 136 | 126.4713 |
| Q15208 | D01cd7 | serine/threonine kinase 38 | 158.8765 | 204 | 126.5253 |
| Q9UET6 | J19gh6 | FisJ homolog 1 (E. coli) | 102.5266 | 120 | 126.5378 |
| Q9H5N0 | F15gh5 | chromosome 7 open reading frar [symbol] | 102.3349 | 129 | 126.5687 |
| Q9H019 | B22gh4 | hypothetical protein RP1-317E23 | 102.5329 | 150 | 126.5974 |
| Q9Y673 | F05cd8 | A1g5; S. cerevisiae; homolog of | 100.2543 | 131 | 126.6884 |
| O75690 | M08gh7 | UHS KerB | 121.3598 | 150 | 126.7819 |
| Q9NUP6 | H22gh2 | hypothetical protein FLJ11220 | 95.908 | 144 | 126.8164 |
| Q14296 | C03cd7 | FAST kinase | 122.4924 | 92.9 | 126.9021 |
| Q9NW55 | B20gh2 | hypothetical protein FLJ10307 | 94.73914 | 141 | 126.9877 |
| O43639 | H01cd3 | NCK adaptor protein 2 | 139.9609 | 79.6 | 127.0293 |
| P17544 | L08ab2 | activating transcription factor 7 | 108.5839 | 112 | 127.0848 |
| O75911 | M01cd5 | short-chain dehydrogenase/redu [symbol] | 156.3658 | 173 | 127.1125 |
| Q06430 | K15ef3 | glucosaminyl (N-acetyl) transfera [symbol] | 94.56995 | 139 | 127.1541 |
| Q13769 | D10cd3 | chromosome 22 open reading fra [symbol] | 122.7812 | 121 | 127.1547 |
| Q9H864 | J14gh5 | hypothetical protein FLJ11021 si [symbol] | 97.58044 | 142 | 127.1909 |
| O95500 | F06cd7 | claudin 14 | 183.0294 | 181 | 127.2815 |
| P14222 | P02cd2 | perforin 1 (pore forming protein) | 99.11893 | 135 | 127.3674 |
| Q9Y4C8 | B06ef2 | KIAA0682 gene product | 105.4769 | 146 | 127.3803 |
| Q9H3H5 | M23ab4 | dolichyl-phosphate (UDP-N-acet [symbol] | 193.8179 | 108 | 127.5134 |
| Q13433 | G05cd8 | LIV-1 protein; estrogen regulate [symbol] | 118.096 | 105 | 127.6623 |
| Q00059 | O07cd2 | transcription factor A; mitochond [symbol] | 111.4615 | 131 | 127.6629 |
| Q15119 | I06ab8 | pyruvate dehydrogenase kinase; | 97.01115 | 135 | 127.6675 |
| O43791 | F03cd3 | speckle-type POZ protein | 111.349 | 114 | 127.7061 |
| Q14206 | P10cd5 | Down syndrome critical region g [symbol] | 119.8147 | 263 | 127.7164 |
| Q92926 | N24cd1 | SWI/SNF related; matrix associa [symbol] | 115.147 | 105 | 127.7503 |
| Q9P288 | G15ef3 | BRCA2 and CDKN1A interacting | 148.9477 | 132 | 127.8623 |
| Q9NZU5 | C16ef4 | LIM and cysteine-rich domains 1 | 164.1438 | 128 | 127.9349 |
| Q13569 | O13cd2 | thymine-DNA glycosylase | 83.16502 | 172 | 127.9545 |
| P48167 | D17ef6 | glycine receptor; beta | 109.5501 | 139 | 127.9738 |
| Q9NQR1 | I02gh4 | PR/SET domain containing prote [symbol] | 154.2185 | 92.7 | 128.0258 |
| O43405 | B23ab4 | coagulation factor C homolog; cc | 103.0764 | 121 | 128.1793 |
| Q9P028 | N24ef2 | CCR4-NOT transcription comple [symbol] | 104.0341 | 133 | 128.2089 |
| Q9Y6E8 | P04cd7 | sirtuin silent mating type informa [symbol] | 109.1684 | 110 | 128.2444 |
| Q9NV65 | H17gh3 | hypothetical protein FLJ10901 | 92.33747 | 188 | 128.3243 |
| Q9H0R8 | M13gh7 | GABA(A) receptor-associated pr [symbol] | 122.091 | 179 | 128.3386 |
| O95817 | P02cd4 | BCL2-associated athanogene 3 | 107.1752 | 114 | 128.4319 |
| P36956 | K10cd2 | sterol regulatory element binding | 140.2284 | 109 | 128.532 |
| P08236 | D05ab3 | glucuronidase; beta | 123.7102 | 114 | 128.6103 |
| Q13425 | D12cd2 | syntrophin; beta 2 (dystrophin-as [symbol] | 106.7241 | 122 | 128.6723 |
| Q14676 | E18gh1 | KIAA0170 gene product | 93.41288 | 145 | 128.7328 |
| P16106 | O12gh6 | H3 histone family; member F | 98.02235 | 136 | 128.7695 |
| Q14773 | J03ab6 | intercellular adhesion molecule 4 | 101.85 | 137 | 128.8098 |
| O14736 | N19gh4 | putative methyltransferase | 124.8741 | 126 | 128.8151 |
| Q9Y297 | N21cd4 | beta-transducin repeat containin [symbol] | 152.2864 | 142 | 128.8641 |
| Q13585 | H16cd4 | G protein-coupled receptor 50 | 121.9424 | 113 | 128.8853 |
| O60858 | L24cd5 | ret finger protein 2 | 110.1643 | 110 | 128.8939 |
| Q9Y2Z6 | E06ef2 | CGI-07 protein | 96.22816 | 139 | 128.9272 |
| Q9UG66 | P16ef7 | hypothetical protein DKFZp586G | 318.3297 | 374 | 128.9795 |
| Q9Y6G5 | N17ef2 | PTD002 protein | 107.7937 | 141 | 129.1324 |
| P98155 | E06cd3 | very low density lipoprotein rece [symbol] | 113.1365 | 104 | 129.1355 |
| Q9NP50 | C07gh5 | TERA protein | 94.63638 | 199 | 129.2792 |
| P28328 | C01ab8 | peroxisomal membrane protein 3 | 321.6292 | 219 | 129.3474 |
| Q969T4 | M24cd6 | ubiquitin-conjugating enzyme E2 | 117.2071 | 100 | 129.3512 |
| P19827 | D18ab6 | inter-alpha (globulin) inhibitor; H [symbol] | 239.4455 | 186 | 129.3613 |
| O15096 | M18ab8 | phosphatidylinositol 4-kinase; ca [symbol] | 117.2574 | 115 | 129.4365 |
| Q9Y5J6 | J16cd7 | fracture callus 1 homolog (rat) | 102.357 | 127 | 129.4503 |
| Q92889 | M24ab4 | excision repair cross-complemen [symbol] | 157.5735 | 97.6 | 129.5255 |
| P08697 | C07ef1 | serine (or cysteine) proteinase in | 138.5253 | 136 | 129.5425 |
| Q9UBX1 | A05cd4 | cathepsin F | 113.4065 | 118 | 129.6557 |
| Q9NPI7 | D22ef1 | hypothetical protein LOC51315 | 240.5378 | 171 | 129.76 |
| P11586 | D14ef7 | methylenetetrahydrofolate dehyd [symbol] | 141.2456 | 106 | 129.8444 |
| O96010 | J15cd4 | LIM domain binding 1 | 90.9506 | 155 | 129.8567 |
| Q13188 | P13cd2 | serine/threonine kinase 3 (STE2 [symbol] | 117.4664 | 135 | 129.8809 |
| Q13845 | I21ab3 | B-cell CLL/lymphoma 7B | 145.9359 | 88.3 | 129.9273 |
| Q9Y5W9 | D07cd8 | sorting nexin 11 | 144.6605 | 198 | 130.0152 |
| O60539 | I02cd7 | Ras association (RalGDS/AF-6) [symbol] | 126.8466 | 102 | 130.0346 |
| O60885 | I02ef3 | bromodomain-containing 4 | 114.9928 | 98.1 | 130.1093 |
| Q9H2B0 | O16gh5 | nucleosome assembly protein 1- [symbol] | 122.5782 | 147 | 130.1112 |
| P51582 | C24ab8 | pyrimidinergic receptor P2Y; G-p | 116.4263 | 109 | 130.158 |
| O00585 | F08cd1 | small inducible cytokine subfamil [symbol] | 168.745 | 216 | 130.1849 |
| Q15131 | B23ef5 | cyclin-dependent kinase (CDC2- [symbol] | 168.6104 | 108 | 130.2201 |
| O75148 | F08cd8 | cryptochrome 2 (photolyase-like) | 86.14249 | 172 | 130.27 |
| Q15691 | G17cd8 | microtubule-associated protein; [symbol] | 173.7024 | 168 | 130.2758 |
| Q15779 | H21ab8 | procollagen (type III) N-endopep [symbol] | 141.4408 | 133 | 130.2905 |
| Q9UHD2 | K14cd8 | TANK-binding kinase 1 | 82.31867 | 172 | 130.2947 |
| Q9NSS3 | K15gh3 | hypothetical protein DKFZp434E | 107.5389 | 115 | 130.3095 |
| Q13797 | I01ef7 | integrin; alpha 9 | 156.9946 | 85 | 130.4752 |
| P46531 | M10ef5 | Notch homolog 1; translocation- [symbol] | 86.01963 | 164 | 130.4897 |
| O43262 | C01cd6 | deleted in lymphocytic leukemia; | 76.79463 | 195 | 130.4943 |
| Q9Y2H6 | k12ef8 | KIAA0970 protein | 170.8655 | 128 | 130.5238 |
| P25788 | J11ab8 | proteasome (prosome; macropah [symbol] | 258.6025 | 285 | 130.5899 |
| Q9BQS8 | D09gh7 | FYVE and coiled-coil domain co [symbol] | 164.6348 | 207 | 130.6451 |
| Q9Y396 | J04ef1 | elongation of very long chain fatt [symbol] | 162.6431 | 82.2 | 130.6722 |
| Q9P0R7 | K06ef2 | hypothetical protein LOC51242 | 126.7302 | 218 | 130.6804 |
| O15269 | F17cd6 | serine palmitoyltransferase; long | 97.42808 | 133 | 130.7429 |
| Q9UFX0 | B02ef2 | calcium binding atopy-related au [symbol] | 233.5089 | 287 | 130.8943 |
| Q9H663 | F24gh5 | likely ortholog of mouse actin-rel [symbol] | 98.1679 | 128 | 130.9258 |
| O75410 | P17cd2 | transforming; acidic coiled-coil cc [symbol] | 224.1188 | 133 | 130.9827 |
| Q9Y5N5 | I14cd8 | putative N6-DNA-methyltransfera [symbol] | 99.34841 | 133 | 131.0924 |
| Q9BQ72 | F02gh7 | hypothetical protein MGC10471 | 106.3824 | 115 | 131.1126 |
| Q9Y303 | E10ef2 | CGI-14 protein | 153.5787 | 101 | 131.2035 |
| Q9P005 | A22ef8 | HSPC159 protein | 341.3083 | 579 | 131.3146 |
| Q9Y3A1 | H19ef2 | DKFZP566O084 protein | 200.4927 | 262 | 131.514 |
| Q15172 | C12cd1 | protein phosphatase 2; regulator | 168.3914 | 140 | 131.5524 |
| Q9BV19 | I19gh6 | hypothetical protein MGC955 | 96.47316 | 141 | 131.6188 |
| Q14534 | E11cd2 | squalene epoxidase | 155.5799 | 71.3 | 131.8107 |
| Q9BS43 | G19gh8 | hypothetical protein MGC12435 | 592.1694 | 121 | 131.9022 |
| P49788 | D24ab8 | retinoic acid receptor responder [symbol] | 97.19637 | 135 | 131.9355 |
| Q9UP83 | M10cd6 | component of oligomeric golgi cc [symbol] | 100.2446 | 141 | 131.938 |
| Q9Y2X0 | N11cd5 | thyroid hormone receptor-associ [symbol] | 116.8982 | 97.9 | 131.9541 |
| Q9Y3E3 | I15ef2 | CGI-145 protein | 94.76733 | 132 | 131.9919 |
| O95183 | J14cd6 | vesicle-associated membrane pr [symbol] | 91.19765 | 157 | 132.0539 |
| Q13242 | N22cd3 | splicing factor; arginine/serine-ri [symbol] | 156.0624 | 141 | 132.0637 |
| Q9Y5R5 | A06gh1 | doublesex and mab-3 related tra [symbol] | 100.8446 | 162 | 132.1528 |
| P04181 | N02ab7 | ornithine aminotransferase (gyra [symbol] | 112.3954 | 106 | 132.1687 |
| Q14254 | B15ab4 | flotillin 2 | 130.1586 | 154 | 132.2038 |
| Q9Y6E6 | P08cd7 | sirtuin silent mating type informa [symbol] | 94.40989 | 136 | 132.2501 |
| Q9H237 | D20gh5 | porcupine | 148.2204 | 258 | 132.2656 |
| Q92785 | N21cd2 | requiern; apoptosis response zin [symbol] | 118.3718 | 108 | 132.3893 |
| O95458 | N11cd2 | tubulin-specific chaperone d | 128.9522 | 100 | 132.4761 |
| P20749 | P23ef6 | B-cell CLL/lymphoma 3 | 264.1174 | 186 | 132.484 |
| Q9H2N8 | D24gh7 | hypothetical protein GL012 | 104.3233 | 160 | 132.5137 |
| Q15020 | M08gh1 | squamous cell carcinoma antige [symbol] | 117.2757 | 147 | 132.5393 |
| P42025 | O01gh1 | ARP1 actin-related protein 1 hon | 243.7979 | 92 | 132.554 |
| P98173 | I23gh5 | 2.19 gene | 113.0426 | 116 | 132.6703 |
| O95295 | C06cd8 | SNARE associated protein snapl [symbol] | 324.3312 | 119 | 132.6986 |
| Q9NVA1 | F15gh3 | chromosome 20 open reading fr [symbol] | 111.7508 | 100 | 132.7232 |
| P35573 | O03ab2 | amylo-1; 6-glucosidase; 4-alpha- | 111.9583 | 106 | 132.7432 |
| Q13126 | K13ab7 | methylthioadenosine phosphoryl [symbol] | 202.8169 | 236 | 132.9211 |
| P43034 | C11ab8 | platelet-activating factor acetylhy [symbol] | 106.8174 | 125 | 132.987 |
| Q01844 | E15ab5 | Ewing sarcoma breakpoint regio [symbol] | 111.2628 | 102 | 133.001 |
| P27482 | E18ab6 | calmodulin-like 3 | 114.7017 | 104 | 133.018 |
| Q9NUW4 | N23gh3 | BRIX | 223.8591 | 77.1 | 133.0208 |
| P78552 | J23ab6 | interleukin 13 receptor; alpha 1 | 118.2476 | 93.9 | 133.0765 |
| P51843 | O24ab2 | nuclear receptor subfamily 0; gr [symbol] | 104.7329 | 112 | 133.1401 |
| P15822 | L23ab5 | human immunodeficiency virus [symbol] | 83.26942 | 160 | 133.2118 |
| Q9BXY9 | O13gh8 | RALBP1 associated Eps domain | 90.84238 | 140 | 133.274 |
| Q9ULB4 | P10ef1 | cadherin 9; type 2 (T1-cadherin) | 275.2974 | 137 | 133.4641 |
| Q9NZZ7 | C08ef8 | HSPC171 protein | 214.9523 | 265 | 133.5412 |
| Q02086 | C09cd2 | Sp2 transcription factor | 101.0989 | 133 | 133.5465 |
| Q9BUV9 | H14gh6 | DKFZP586J0119 protein | 102.1174 | 119 | 133.5923 |
| Q9UHJ9 | A09ef4 | FGF receptor activating protein 1 | 100.7903 | 121 | 133.6008 |
| Q16401 | E11cd1 | proteasome (prosome; macropa [symbol] | 143.6091 | 120 | 133.6156 |
| Q9Y4K4 | D04cd6 | mitogen-activated protein kinase | 132.4072 | 158 | 133.6676 |
| O60463 | H06cd3 | phosphalidic acid phosphatase [symbol] | 103.2417 | 151 | 133.6898 |
| O95639 | P20cd6 | cleavage and polyadenylation sp | 183.1762 | 67.6 | 133.7241 |
| P50539 | N19ab7 | MAX interacting protein 1 | 175.6646 | 187 | 133.7304 |
| Q9UH62 | I15ef3 | ALEX3 protein | 118.2527 | 171 | 133.7314 |
| O15525 | C13ab7 | v-maf musculoaponeurotic fibros | 105.3252 | 109 | 133.7521 |
| Q9H8T0 | H21gh5 | fused toes homolog (mouse) | 106.2145 | 241 | 133.7606 |
| Q9Y4X5 | K03cd6 | ariadne homolog; ubiquitin-conju | 99.46477 | 128 | 133.8771 |
| Q9H9E1 | O01gh5 | ankyrin repeat; family A (RFXAN | 172.6266 | 78.8 | 134.052 |
| Q93063 | F13ab3 | exostoses (multiple) 2 | 123.2886 | 161 | 134.3466 |
| Q9H992 | D22gh5 | axotrophin | 89.54768 | 149 | 134.4784 |
| O43237 | P02ab4 | dynein; cytoplasmic; light interm [symbol] | 290.6769 | 119 | 134.4914 |
| Q99643 | H02cd1 | succinate dehydrogenase compl [symbol] | 143.4474 | 125 | 134.5527 |
| P24386 | F11ef1 | choroideremia (Rab escort protei [symbol] | 93.86058 | 129 | 134.5538 |
| Q9Y5F7 | F12gh4 | protocadherin gamma subfamily | 101.7878 | 114 | 134.5585 |
| P06756 | D14ab6 | integrin: alpha V (vitronectin rec [symbol] | 200.0782 | 219 | 134.6115 |
| Q9H5L7 | C02gh6 | chromosome 1 open reading frar [symbol] | 173.8106 | 148 | 134.6601 |
| O15517 | A14ef6 | tumor necrosis factor receptor su [symbol] | 92.33364 | 207 | 134.6603 |
| Q9P0N5 | K24ef2 | HSPC244 | 107.9991 | 129 | 134.6618 |
| Q9NWT2 | D19gh2 | hypothetical protein FLJ20623 | 107.8864 | 105 | 134.7112 |
| Q9Y2A0 | O02cd7 | TP53 target gene 1 | 127.408 | 88.2 | 134.8491 |
| Q9NZ43 | N16gh4 | uncharacterized hematopoietic s [symbol] | 141.2548 | 137 | 134.9524 |
| P27986 | A10ef1 | phosphoinositide-3-kinase; regul [symbol] | 111.3806 | 112 | 134.9586 |
| Q9UBP6 | J13ef1 | methyltransferase-like 1 | 107.2187 | 122 | 134.986 |
| Q9BWK5 | P24gh5 | hypothetical protein MGC5242 | 101.1324 | 123 | 134.9881 |
| P54802 | M24ab6 | N-acetylglucosaminidase; alpha- | 206.038 | 66.3 | 135.077 |
| O75963 | H09cd7 | G-protein coupled receptor | 110.3886 | 184 | 135.3412 |
| Q9NXZ4 | F03ef4 | ELG protein | 124.5205 | 105 | 135.3934 |
| O60353 | B11cd3 | frizzled homolog 6 (Drosophila) | 85.64918 | 146 | 135.5169 |
| Q9NQS6 | F13gh4 | latexin protein | 94.21822 | 163 | 135.5761 |
| O14776 | A19cd7 | transcription elongation regulator | 113.3803 | 117 | 135.6603 |
| Q9Y5A2 | L13ef1 | putative zinc finger protein NY-R | 154.0704 | 186 | 135.6708 |
| P49842 | G23cd6 | serine/threonine kinase 19 | 133.3557 | 171 | 135.7013 |
| Q06546 | J14ab5 | GA binding protein transcription [symbol] | 88.93312 | 138 | 135.7488 |
| Q9Y2E7 | I02ef8 | KIAA0938 protein | 109.8218 | 105 | 135.7763 |
| Q9UM11 | I02ef2 | Fzr1 protein | 80.82847 | 186 | 135.8949 |
| Q99447 | B14ab8 | phosphate cytidylyltransferase 2; | 174.8201 | 77.4 | 135.988 |
| Q9H1R3 | F22gh8 | myosin light chain kinase 2; skel [symbol] | 93.58977 | 129 | 136.0456 |
| P28358 | P05ab6 | homeo box D10 | 108.9905 | 102 | 136.1419 |
| O76070 | P18cd1 | synuclein; gamma (breast cancer | 128.551 | 140 | 136.1645 |
| Q9Y5A7 | N07ef2 | NEDD8 ultimate buster-1 | 119.0653 | 195 | 136.2606 |
| Q9NWY7 | A16gh3 | hypothetical protein FLJ20530 | 103.7638 | 110 | 136.3026 |
| O43715 | P02ef2 | hypothetical protein HSPC132 | 137.4694 | 110 | 136.3043 |
| Q9BY75 | K19gh8 | itchy homolog E3 ubiquitin protei [symbol] | 118.5721 | 127 | 136.5094 |
| O43431 | A12ef7 | endothelial differentiation; lysop [symbol] | 126.4659 | 171 | 136.5393 |
| O43567 | D15cd7 | ring finger protein 13 | 110.4449 | 153 | 136.5722 |
| Q9Y2S7 | P13ef8 | DKFZP586F1524 protein | 113.1366 | 104 | 136.7041 |
| O14582 | O24ef2 | spondyloepiphyseal dysplasia; la [symbol] | 135.1666 | 135 | 136.7342 |
| O75056 | G12gh1 | syndecan 3 (N-syndecan) | 120.0661 | 162 | 136.797 |
| Q9P2Y2 | D18gh4 | beta-1; 4 mannosyltransferase | 89.6857 | 143 | 136.8208 |
| Q16595 | M08ab3 | Friedreich alaxia | 139.5657 | 130 | 136.8314 |
| Q14807 | B18ab7 | kinesin-like 4 | 114.4726 | 94.2 | 136.9298 |
| Q9BZL4 | L10gh6 | protein phosphatase 1; regulator | 110.5367 | 123 | 137.0777 |
| P30876 | I13ab8 | polymerase (RNA) II (DNA direct [symbol] | 265.5337 | 200 | 137.1153 |
| Q9UGP6 | B17cd7 | coatomer protein complex; subu [symbol] | 94.73601 | 134 | 137.3091 |
| P52655 | M01ef2 | general transcription factor IIA; 1 [symbol] | 121.4896 | 128 | 137.3707 |
| Q9HB07 | G23gh5 | chromosome 12 open reading fr [symbol] | 121.2547 | 118 | 137.4723 |
| Q92940 | H23ab7 | MAD; mothers against decapent [symbol] | 414.4889 | 223 | 137.6028 |
| Q9NWN1 | H19gh2 | hypothetical protein FLJ20727 | 117.7514 | 120 | 137.6351 |
| O43664 | A10cd6 | G protein-coupled receptor 66 | 108.1311 | 102 | 137.6789 |
| O14647 | C19ab4 | chromodomain helicase DNA bin [symbol] | 96.14904 | 164 | 137.7441 |
| O75333 | N15cd2 | T-box 10 | 126.0065 | 129 | 137.7737 |
| Q16539 | J04ef5 | mitogen-activated protein kinase | 148.7339 | 219 | 137.8 |
| Q9NQG5 | A07gh5 | chromosome 20 open reading fr [symbol] | 86.80552 | 143 | 137.803 |
| Q15149 | E05ab8 | plectin 1; intermediate filament bi [symbol] | 125.7248 | 83.9 | 138.0785 |
| Q9NVZ7 | G12gh3 | hypothitical protein FLJ10407 | 166.213 | 175 | 138.1444 |
| Q15800 | D02cd2 | sterol-C4-methyl oxidase-like | 171.8648 | 71.2 | 138.1497 |
| Q9H0B6 | D16gh5 | hypothetical protein FLJ12387 si [symbol] | 96.54943 | 129 | 138.1542 |
| P09001 | K14cd7 | mitochondrial ribosomal protein [symbol] | 94.17331 | 128 | 138.1601 |
| O00301 | D24cd3 | KH-type splicing regulatory prote [symbol] | 126.0509 | 88.3 | 138.3014 |
| P55786 | J19ef7 | aminopeptidase puromycin sensi [symbol] | 273.7328 | 98.9 | 138.3924 |
| Q9Y5Y9 | B14cd2 | sodium channel; voltage-gated; t [symbol] | 127.3727 | 139 | 138.4331 |
| P19235 | M0 [symbol] ef7 | erythropoietin receptor | 198.5954 | 77.3 | 138.4803 |
| O15228 | A16ef3 | glyceronephosphate O-acyltrans [symbol] | 225.1315 | 186 | 138.5137 |
| Q9UK97 | K05cd8 | F-box only protein 9 | 151.0501 | 104 | 138.5593 |
| P50990 | D20cd6 | chaperonin containing TCP1; sut [symbol] | 274.982 | 178 | 138.6001 |
| Q9NZM1 | P19cd8 | fer-1-like 3; myoferlin (C. elegan [symbol] | 218.8411 | 202 | 138.6209 |
| Q92924 | N20cd1 | SWI/SNF related; matrix associa [symbol] | 131.7669 | 82.7 | 138.6764 |
| O75845 | A18gh1 | sterol-C5-desaturase (ERG3 dell | 101.9285 | 153 | 138.7232 |
| Q9NQ88 | I10gh4 | chromosome 12 open reading fr [symbol] | 80.2401 | 158 | 138.8274 |
| Q16515 | C19ab2 | amiloride-sensitive cation chann [symbol] | 102.1159 | 109 | 138.9915 |
| P49759 | F16ab5 | CDC-like kinase 1 | 86.24615 | 147 | 139.0985 |
| P33552 | A22ab5 | CDC28 protein kinase 2 | 109.5671 | 96 | 139.1297 |
| O75808 | H21cd2 | small optic lobes homolog (Dros [symbol] | 115.654 | 98.9 | 139.1625 |
| Q9NXR1 | M07gh2 | LIS1-interacting protein NUDE1; | 96.20481 | 123 | 139.1939 |
| O75381 | L12ab8 | peroxisomal biogenesis factor 14 | 473.8395 | 113 | 139.2209 |
| O60930 | P23cd1 | ribonuclease H1 | 167.3611 | 111 | 139.2393 |
| O14653 | P14cd4 | golgi SNAP receptor complex me [symbol] | 115.7917 | 89.7 | 139.2541 |
| Q16540 | L24gh1 | mitochondrial ribosomal protein [symbol] | 209.2433 | 120 | 139.3426 |
| O14658 | P06cd2 | ras-related C3 botulinum toxin s [symbol] | 158.176 | 166 | 139.3634 |
| Q15814 | M17cd2 | tubulin-specific chaperone c | 103.3699 | 112 | 139.6311 |
| Q9Y3C9 | J09ef2 | CGI-127 protein | 83.4762 | 163 | 139.6599 |
| P18827 | F22cd1 | syndecan 1 | 114.4045 | 92.7 | 139.6607 |
| P04150 | B21ab3 | nuclear receptor subfamily 3; gro [symbol] | 106.6921 | 103 | 139.6889 |
| Q9Y301 | N05ef1 | CGI-12 protein | 108.0511 | 101 | 139.7035 |
| O00626 | F10cd1 | small inducible cytokine subfamil [symbol] | 113.478 | 106 | 139.8189 |
| | B18cd8 | neural precursor cell expressed; | 108.1711 | 133 | 139.9597 |
| Q9Y311 | C01ef2 | mitochondrial ribosomal protein 1 | 162.8519 | 74.8 | 139.9856 |
| Q15633 | K14cd2 | TAR (HIV) RNA binding protein [symbol] | 169.3779 | 125 | 140.0138 |
| P02278 | M22gh6 | H2B histone family; member K | 100.2794 | 216 | 140.0965 |
| P53672 | J11cd7 | crystallin; beta A2 | 101.7321 | 187 | 140.2804 |
| P09417 | C05ab8 | quinoid dihydropteridine reducta [symbol] | 126.6361 | 161 | 140.3564 |
| Q9HAY2 | K14gh5 | MAGEF1 protein | 96.01675 | 126 | 140.3654 |
| P30273 | A20ab5 | Fc fragment of IgE; high affinity I | 88.48289 | 171 | 140.4541 |
| Q92949 | G10ab4 | forkhead box J1 | 250.4112 | 167 | 140.5084 |
| Q9GZT9 | G01gh7 | egl nine homolog 1 (C. elegans) | 112.8025 | 99.2 | 140.6556 |
| P35658 | O23cd4 | nucleoporin 214 kD (CAIN) | 141.8279 | 137 | 140.7761 |
| Q9HB40 | G01gh5 | likely homolog of rat mouse [symbol] | 105.9316 | 99.3 | 140.9548 |
| O14686 | O16cd3 | myeloid/lymphoid or mixed-linea [symbol] | 117.844 | 157 | 141.0674 |
| Q92911 | O12cd1 | solute carrier family 5 (sodium io [symbol] | 264.1256 | 125 | 141.0904 |
| Q9Y5E4 | B18ef3 | protocadherin beta 5 | 95.08061 | 148 | 141.1894 |
| Q13057 | H17gh7 | nucleotide binding protein | 186.8796 | 125 | 141.2202 |
| Q14863 | B23ab8 | POU domain; class 6; transcripti [symbol] | 103.2077 | 103 | 141.3258 |
| P36610 | G10ef3 | frequenin homolog (Drosophila) | 110.2191 | 136 | 141.4925 |
| O60240 | O12ab8 | perilipin | 1121.108 | 52.2 | 141.5046 |
| Q9H9B4 | P05gh5 | hypothetical protein FLJ12876 | 87.99841 | 133 | 141.7125 |
| O00584 | J14cd3 | ribonuclease 6 precursor | 124.6932 | 250 | 141.8008 |
| Q9H658 | M04gh7 | G protein-coupled receptor 107 | 158.2595 | 214 | 141.8139 |
| P18858 | K14ef6 | ligase I; DNA; ATP-dependent | 92.33071 | 128 | 141.8761 |
| Q02877 | D23cd1 | ribosomal protein L26 | 449.1728 | 297 | 141.8851 |
| P15586 | J07ab4 | glucosamine (N-acetyl)-6-sulfata [symbol] | 261.466 | 119 | 141.968 |
| P42696 | H08ef4 | KIAA0117 protein | 110.9202 | 231 | 141.9948 |
| O75710 | A17ef1 | molybdenum cofactor synthesis [symbol] | 105.3087 | 101 | 142.0352 |
| Q9NPI8 | C01gh2 | Fanconi anemia; complementatic | 193.8104 | 214 | 142.0643 |
| Q9HAR6 | O10gh5 | uterine-derived 14 kDa protein | 118.9551 | 198 | 142.0669 |
| P15018 | O18ef7 | leukemia inhibitory factor (cholin [symbol] | 253.8099 | 309 | 142.2733 |
| Q13546 | A23cd4 | receptor (TNFRSF)-interacting s [symbol] | 139.7074 | 131 | 142.2746 |
| P24298 | H01ab4 | glutamic-pyruvate transaminase [symbol] | 210.426 | 97.1 | 142.3123 |
| Q14129 | C22cd4 | DiGeorge syndrome critical regio [symbol] | 112.1253 | 141 | 142.4302 |
| Q9BQA2 | K09gh8 | KIAA1882 protein | 164.9357 | 187 | 142.4325 |
| Q9UJX9 | B12ef3 | hypothetical protein; estradiol-in [symbol] | 121.7396 | 98.5 | 142.5053 |
| Q99590 | I01cd5 | splicing factor; arginine/serine-ri [symbol] | 231.0328 | 199 | 142.5273 |
| O14798 | C06ef7 | tumor necrosis factor receptor su | 142.6951 | 213 | 142.5294 |
| P32238 | P01ab3 | cholecystokinin A receptor | 487.368 | 168 | 142.5911 |
| Q9UQ90 | C17cd2 | spastic paraplegia 7; paraplegin | 101.2857 | 146 | 142.6321 |
| P82980 | M03gh8 | retinol binding protein 5; cellular | 111.2191 | 93.9 | 142.6923 |
| P49716 | P15ab5 | CCAAT/enhancer binding protei [symbol] | 169.1889 | 73.4 | 142.8277 |
| P20062 | M12cd1 | transcobalamin II; macrocytic an [symbol] | 125.6347 | 141 | 142.8402 |
| P21580 | B06cd2 | tumor necrosis factor; alpha-indu [symbol] | 275.8225 | 317 | 142.8664 |
| Q99774 | H01cd5 | protein phosphatase 4; regulator | 195.85 | 173 | 143.1867 |
| Q9P0T1 | A17ef3 | hypothetical protein HSPC192 | 104.4363 | 101 | 143.2517 |
| P06737 | B18ab8 | phosphorylase; glycogen; liver ( [symbol] | 85.14818 | 139 | 143.2732 |
| O15063 | K10gh1 | KIAA0355 gene product | 149.9191 | 144 | 143.2945 |
| P02743 | K14ab2 | amyloid P component; serum | 108.0152 | 94.7 | 143.3031 |
| Q9NVT0 | I14gh3 | hypothetical protein FLJ10534 | 136.4264 | 109 | 143.3056 |
| Q9Y4P3 | O07ef3 | transducin (beta)-like 2 | 112.0852 | 104 | 143.4853 |
| Q13886 | N14ab3 | basic transcription element bindi [symbol] | 164.4049 | 88.7 | 143.5269 |
| Q9NVP8 | L19gh4 | hypothetical protein FLJ10595 | 101.7956 | 106 | 143.5829 |
| P08240 | G05cd2 | signal recognition particle recept [symbol] | 158.3565 | 132 | 143.6823 |
| P49459 | M19ef5 | ubiquitin-conjugating enzyme E2 | 98.6462 | 170 | 143.74 |
| Q9NVAB | L16gh2 | solute carrier family 38; member | 112.6488 | 127 | 143.7595 |
| P25325 | K23cd3 | mercaptopyruvate sulfurtransfer [symbol] | 288.1435 | 274 | 143.8077 |
| Q9UHI6 | K08cd7 | DEAD/H (Asp-Glu-Ala-Asp-His) t [symbol] | 135.6699 | 209 | 143.8383 |
| P52298 | H01cd7 | nuclear cap binding protein subu [symbol] | 138.9223 | 191 | 143.8819 |
| Q9NQ30 | A10cd7 | endothelial cell-specific molecut [symbol] | 104.1846 | 122 | 143.9703 |
| O00541 | I08ef3 | pescadillo homolog 1; containing | 180.43 | 204 | 144.0988 |
| Q9H2V9 | B06gh7 | hypothetical protein CDA08 | 173.5608 | 169 | 144.1057 |
| Q9NNX1 | F12gh1 | tuftelin 1 | 147.0323 | 175 | 144.106 |
| Q13508 | H14ab2 | ADP-ribosyltransferase 3 | 109.4052 | 101 | 144.1545 |
| Q9NR30 | B21gh6 | DEADE/H (Asp-Glu-Ala-Asp/His) t | 110.4836 | 130 | 144.2348 |
| Q14320 | E15cd5 | DNA segment on choromosome X | 102.5057 | 178 | 144.2677 |
| Q04726 | D03cd2 | transducin-like enhancer of split [symbol] | 211.8773 | 115 | 144.3217 |
| P20061 | N13cd1 | transcobalamin I (vitamin B12 bi [symbol] | 177.3445 | 85.5 | 144.4835 |
| Q9Y2Y1 | J12ef2 | polymerase (RNA) III (DNA direc [symbol] | 153.7426 | 145 | 144.5561 |
| P35754 | G22ab5 | glutaredoxin (thioltransferase) | 153.823 | 138 | 144.7308 |
| Q02978 | F01cd3 | solute carrier family 25 (mitocho [symbol] | 106.877 | 98.2 | 144.7822 |
| O95792 | N20gh2 | protein associated with PRK1 | 130.6914 | 146 | 144.7909 |
| Q13232 | D20ef7 | non-metastatic cells 3; protein e [symbol] | 90.7906 | 156 | 144.8861 |
| Q9NVH2 | L01ef8 | DKFZP434B168 protein | 117.6587 | 91.2 | 144.9821 |
| Q15561 | O15cd2 | TEA domain family member 4 | 234.306 | 172 | 145.3307 |
| Q9UPN9 | B01ef2 | tripartite motif-containing 33 | 110.9269 | 135 | 145.3321 |
| Q14353 | O06ab3 | guanidinoacetate N-methyltrans [symbol] | 109.1385 | 103 | 145.3798 |
| Q9NZM5 | A18ef4 | glioma tumor suppressor candid [symbol] | 150.098 | 122 | 145.587 |
| O43255 | B11cd2 | seven in absentia homolog 2 (D [symbol] | 114.6318 | 101 | 145.653 |
| Q14331 | G04ab5 | FSHD region gene 1 | 164.0964 | 176 | 145.6576 |
| P18847 | N23ab5 | activating transcription factor 3 | 127.5442 | 154 | 145.6654 |
| O60258 | I07cd4 | fibroblast growth factor 17 | 159.7671 | 223 | 145.6821 |
| P47929 | P02ab6 | lectin; galactoside-binding; solub | 151.1595 | 92.8 | 145.699 |
| P35227 | O14cd4 | zinc finger protein 144 (Mel-18) | 108.2295 | 151 | 145.8554 |
| Q15599 | O15cd5 | solute carrier family 9 (sodium/h [symbol] | 168.9673 | 80.5 | 145.993 |
| Q9UKJ5 | B20cd7 | cystein-rich hydrophobic domain | 184.4717 | 206 | 145.9967 |
| Q9HCB9 | C10gh5 | chromosome 1 open reading frar [symbol] | 94.16759 | 126 | 146.1683 |
| Q9Y3A0 | K07ef2 | CGI-92 protein | 119.1033 | 178 | 146.1698 |
| P49815 | M17ef6 | tuberous sclerosis 2 | 106.0159 | 163 | 146.2712 |
| P25116 | L19ab4 | coagulation factor II (thrombin) r [symbol] | 82.48228 | 143 | 146.2742 |
| O75829 | O01cd7 | chondromodulin I precursor | 121.7658 | 119 | 146.4876 |
| Q9NYI1 | M16ef2 | ECSIT | 111.7926 | 110 | 146.6345 |
| P24347 | I24ef5 | matrix metalloproteinase 11 (stro [symbol] | 97.57916 | 136 | 146.649 |
| O43189 | K24ab8 | PHD finger protein 1 | 112.1101 | 101 | 146.6925 |
| Q9P029 | N22ef2 | TH1-like (Drosophila) | 201.2292 | 159 | 146.6998 |
| Q9NZP9 | E17ef2 | immediate early response 5 | 105.1683 | 157 | 146.761 |
| Q9UIA3 | I12ef2 | neutral sphingomyelinase | 165.6408 | 147 | 146.8134 |
| Q99734 | M12ef5 | Notch homolog 2 (Drosophila) | 220.4251 | 263 | 147.0111 |
| O00411 | C19cd1 | polymerase (RNA) mitochondrial | 120.5078 | 105 | 147.0481 |
| P28347 | P04gh1 | TEA domain family member 1 (S [symbol] | 230.2101 | 174 | 147.0913 |
| Q13619 | G19gh1 | cullin 4A | 153.1592 | 218 | 147.2043 |
| O94929 | O06ef8 | KIAA0843 protein | 108.7821 | 99.4 | 147.2921 |
| Q9P026 | A02ef8 | HSPC134 protein | 115.4895 | 184 | 147.3205 |
| P28067 | D08ab5 | major histocompatibility complex; | 128.7098 | 100 | 147.3934 |
| Q14126 | E11ef7 | desmoglein 2 | 426.8387 | 366 | 147.4196 |
| Q14267 | P01cd1 | regulatory factor X: 4 (influences | 127.7912 | 98.9 | 147.4351 |
| P55197 | H09ab2 | myeloid/lymphoid or mixed-linea [symbol] | 160.668 | 185 | 147.4439 |
| Q9NP87 | O20cd8 | polymerase (DNA directed); mu [symbol] | 115.1765 | 86.7 | 147.4845 |
| P04155 | A04cd2 | trefoil factor 1 (breast cancer; es [symbol] | 258.058 | 136 | 147.487 |
| Q9C069 | N02gh7 | sec13-like protein | 52.65015 | 424 | 147.5255 |
| P12270 | G14cd2 | translocated promoter region (to | 104.4493 | 136 | 147.7447 |
| Q9UIG6 | I14ef2 | LPAP for lysophosphatidic acid [symbol] | 218.3106 | 325 | 147.8779 |
| O15453 | P08cd5 | NBR2 | 99.08545 | 108 | 147.9143 |
| Q9UNW8 | F17cd8 | G protein-coupled receptor | 239.3255 | 347 | 148.021 |
| P06746 | I10ef6 | polymerase (DNA directed); beta | 142.5013 | 225 | 148.0351 |
| O15414 | I08cd7 | trinucleotide repeat containing 4 | 131.6219 | 199 | 148.0947 |
| O15259 | L22ab7 | nephronophthisis 1 (juvenile) | 115.0436 | 110 | 148.3613 |
| Q9NRA8 | P05gh4 | eIF4E-transporter | 106.343 | 178 | 148.4323 |
| Q9NP77 | C10ef8 | HSPC182 protein | 171.17 | 175 | 148.5234 |
| Q06587 | P17cd1 | ring finger protein 1 | 119.2377 | 127 | 148.657 |
| Q9Y5Z5 | E22ef2 | heme binding protein 1 | 181.0768 | 196 | 148.7681 |
| O15327 | I05cd4 | inositol polyphosphate-4-phosph [symbol] | 183.6815 | 131 | 148.785 |
| P49646 | P19cd1 | regulator of mitotic spindle assen [symbol] | 143.9463 | 117 | 148.8487 |
| Q99809 | F16cd5 | conserved gene amplified in oste [symbol] | 134.0126 | 91.3 | 148.9901 |
| Q9UBM1 | G02cd7 | phosphatidylethanolamine N-mel [symbol] | 138.4849 | 122 | 149.0109 |
| P16403 | G04ab6 | H1 histone family; member 2 | 427.1258 | 558 | 149.1016 |
| O75503 | B01ab4 | ceroid-lipofuscinosis; neuronal 5 | 148.9991 | 75.4 | 149.1361 |
| P15814 | P20gh6 | immunoglobulin lambda-like poly [symbol] | 78.91644 | 144 | 149.2005 |
| Q03164 | N01ab7 | myeloid/lymphoid or mixed-linea [symbol] | 157.0713 | 81.9 | 149.358 |
| Q13624 | B08ef6 | interleukin enhancer binding fact [symbol] | 136.4076 | 130 | 149.4265 |
| Q14118 | M02ab5 | dystroglycan 1 (dystrophin-assoc [symbol] | 105.7987 | 99 | 149.6192 |
| Q13286 | E20ab3 | ceroid-lipofuscinosis; neuronal 3; | 240.521 | 302 | 149.6542 |
| Q9Y5Q0 | E15gh7 | fatty acid desaturase 3 | 117.0825 | 115 | 149.704 |
| Q9P0R1 | K08ef2 | forkhead box P1 | 99.17408 | 114 | 149.7517 |
| P41182 | C24ab5 | B-cell CLL/lymphoma 6 (zinc fing [symbol] | 115.9704 | 85.9 | 149.853 |
| O75497 | K14cd6 | microspherule protein 1 | 145.7927 | 163 | 149.9313 |
| Q14185 | M19ab4 | dedicator of cyto-kinesis 1 | 95.3322 | 112 | 150.0398 |
| O95163 | P05cd3 | inhibitor of kappa light polypeptic [symbol] | 203.0161 | 120 | 150.0489 |
| Q14508 | L05ef7 | WAP four-disulfide core domain: | 102.6084 | 201 | 150.0782 |
| P25942 | M21ef7 | tumor necrosis factor receptor su [symbol] | 110.0933 | 239 | 150.083 |
| Q9Y6E0 | F15cd3 | serine/threonine kinase 24 (STE: | 206.7079 | 225 | 150.279 |
| Q9UQF2 | J21cd5 | mitogen-activated protein kinase | 229.9384 | 182 | 150.2902 |
| Q9Y285 | D11ab5 | phenylalanine-tRNA synthetase- [symbol] | 179.7613 | 145 | 150.3716 |
| Q04762 | F09ab4 | cell matrix adhesion regulator | 134.1594 | 176 | 150.3997 |
| Q9Y6J9 | P19ef6 | TAF6-like RNA polymerase II; p3 | 77.59128 | 158 | 150.4181 |
| O43913 | C06ab8 | origin recognition complex; subu [symbol] | 147.9187 | 168 | 150.4866 |
| P25208 | M05cd1 | nuclear transcription factor Y; be [symbol] | 106.4862 | 138 | 150.5231 |
| Q9H9X4 | F03gh5 | hypothetical protein FLJ11618 | 189.58 | 179 | 150.5879 |
| O95297 | P17cd4 | myelin protein zero-like 1 | 127.461 | 121 | 150.7509 |
| Q9UPG8 | O06ab8 | pleiomorphic adenoma gene-like | 134.8747 | 165 | 150.9533 |
| P16333 | H16ef5 | NCK adaptor protein 1 | 237.1463 | 351 | 150.9549 |
| Q14108 | I22ab5 | CD36 antigen (collagen type I re [symbol] | 155.206 | 139 | 150.9785 |
| Q9UFF9 | O05cd5 | CCR4-NOT transcription comple [symbol] | 162.9025 | 208 | 151.0687 |
| O15242 | O19ab8 | nardilysin (N-arginine dibasic co [symbol] | 210.0164 | 198 | 151.1245 |
| Q13442 | N15ef3 | PDGFA associated protein 1 | 156.1376 | 188 | 151.1314 |
| Q99755 | A15ef6 | phosphatidylinositol-4-phosphat [symbol] | 112.2177 | 146 | 151.3432 |
| Q9NZV1 | E09gh2 | cysteine-rich motor neuron 1 | 112.5932 | 99.1 | 151.3541 |
| Q05195 | C09ab7 | MAX dimerization protein | 136.8996 | 254 | 151.5662 |
| O95671 | J14ab2 | acetylserotonin O-methyltransfer [symbol] | 99.67178 | 98.5 | 151.7064 |
| Q9Y658 | A09ef2 | RNA helicase | 112.9715 | 95.3 | 151.8988 |
| Q9BTU6 | N12gh6 | phosphatidylinositol 4-kinase typ [symbol] | 97.30862 | 178 | 151.965 |
| Q9UPN6 | G04ef8 | KIAA1116 protein | 127.681 | 113 | 152.0268 |
| O00170 | J03ab2 | aryl hydrocarbon receptor intera [symbol] | 555.1127 | 223 | 152.0508 |
| Q9UJW6 | P01ef3 | activity-regulated cytoskeleton-a [symbol] | 221.8559 | 185 | 152.1474 |
| Q9HCT0 | N02ef8 | fibroblast growth factor 22 | 157.7471 | 126 | 152.2042 |
| Q15459 | O03cd6 | splicing factor 3a; subunit 1; 120 [symbol] | 163.4432 | 145 | 152.3945 |
| O95400 | G16cd6 | CD2 antigen (cytoplasmic tail) bi [symbol] | 192.2313 | 238 | 152.5257 |
| P07954 | M06ab3 | fumarate hydratase | 121.2546 | 142 | 152.5652 |
| Q9H6Z5 | C16gh5 | hypothetical protein FLJ21634 | 101.1467 | 152 | 152.6745 |
| Q9HCN8 | A05gh1 | stromal cell-derived factor 2-like | 136.5604 | 128 | 152.9097 |
| Q92890 | A24cd4 | ubiquitin fusion degradation 1-lik [symbol] | 157.2559 | 220 | 152.9538 |
| O43324 | N24cd4 | eukaryotic translation elongation | 150.6748 | 112 | 152.9613 |
| Q9NVU8 | H10gh2 | hypothetical protein FLJ10496 | 145.4398 | 119 | 152.9651 |
| Q9NQW6 | H01ef4 | anillin; actin binding protein (scr [symbol] | 113.8655 | 85.6 | 152.992 |
| Q9NWH4 | L01gh2 | mitochondrial ribosomal protein [symbol] | 173.4347 | 107 | 153.0783 |
| Q9UBV8 | M13cd8 | PEF protein with a long N-termin [symbol] | 146.0371 | 144 | 153.0828 |
| P15328 | F10ab3 | folate receptor 1 (adult) | 152.5051 | 123 | 153.1163 |
| Q9Y583 | B01cd6 | NS1-associated protein 1 | 143.6283 | 177 | 153.2797 |
| Q07326 | M08ab8 | phosphatidylinositol glycan; clas [symbol] | 132.6521 | 93 | 153.3084 |
| O75396 | M12cd5 | SEC22 vesicle trafficking protein [symbol] | 143.9018 | 153 | 153.3626 |
| Q9Y2Z8 | D09ef2 | CGI-09 protein | 117.2943 | 110 | 153.5282 |
| Q9H3P7 | N05gh5 | golgi phosphoprotein 1 | 251.3681 | 258 | 153.65 |
| O95678 | E03cd5 | cytokeratin type II | 339.3692 | 195 | 153.797 |
| P17026 | M04cd4 | zinc finger protein 22 (KOX 15) | 152.726 | 88.3 | 153.8779 |
| Q9Y3N9 | M05gh7 | olfactory receptor; family 2; subf [symbol] | 127.2527 | 137 | 153.9228 |
| Q9BYT8 | K08gh7 | neurolysin (metallopeptidase M3 | 85.99781 | 193 | 154.0005 |
| Q9UHQ7 | F02ef8 | pp21 homolog | 119.2248 | 136 | 154.0057 |
| O60921 | A22ab7 | HUS1 checkpoint homolog (S. p [symbol] | 148.5718 | 204 | 154.126 |
| O00400 | D05ab2 | acetyl-Coenzyme A transporter | 196.9505 | 124 | 154.2012 |
| Q9Y690 | H01gh6 | mortality factor 4 | 253.2426 | 259 | 154.2307 |
| Q9NZL9 | L24ef7 | methionine adenosyltransferase | 128.4144 | 99.7 | 154.2681 |
| O60563 | A05ab4 | cyclin T1 | 179.5706 | 171 | 154.2911 |
| P20591 | M03ab7 | myxovirus (influenza virus) resist | 459.0611 | 873 | 154.338 |
| Q9BQC6 | P14gh5 | mitochondrial ribosomal protein [symbol] | 188.6422 | 74.3 | 154.357 |
| P28838 | J02ef1 | leucine aminopeptidase 3 | 409.1007 | 383 | 154.3828 |
| Q9NZE5 | P08ef1 | hypothetical protein LOC51319 | 135.1502 | 171 | 154.3904 |
| Q9NX47 | K10gh2 | hypothetical protein FLJ20445 | 299.3918 | 338 | 154.4327 |
| O60612 | K11cd2 | supervillin | 113.628 | 270 | 154.4497 |
| O00559 | F22cd4 | estrogen receptor binding site as | 181.5726 | 147 | 154.679 |
| Q9NUU9 | E17gh3 | hypothetical protein F23149_1 | 140.3785 | 224 | 154.7106 |
| P22059 | C10ab8 | oxysterol binding protein | 133.2924 | 93.6 | 154.8714 |
| O00408 | I05ef6 | phosphodiesterase 2A; cGMP-sti | 860.2983 | 169 | 154.9688 |
| O94935 | P22ef7 | SAC1 suppressor of actin mutati [symbol] | 146.0417 | 128 | 154.9914 |
| P41134 | H15ef5 | inhibitor of DNA binding 1; domi [symbol] | 434.1844 | 292 | 155.0439 |
| P24407 | M08ab7 | mel transforming oncogene (deri [symbol] | 224.1153 | 189 | 155.1684 |
| O00232 | N05ab8 | proteasome (prosome; macropa [symbol] | 122.517 | 139 | 155.2465 |
| Q01432 | G04ab2 | adenosine monophosphate dea [symbol] | 111.1768 | 117 | 155.341 |
| Q13525 | G15cd4 | interleukin 1 receptor-like 2 | 136.0802 | 130 | 155.4674 |
| Q16611 | N04ab3 | BCL2-antagonist/killer 1 | 280.4131 | 194 | 155.5414 |
| Q9NPA3 | C11gh5 | hypothetical protein STRAIT114 [symbol] | 112.5378 | 85.5 | 155.5531 |
| Q9GZM8 | D14gh7 | LIS1-interacting protein NUDEL; | 137.8235 | 131 | 155.5611 |
| P42702 | P06ab6 | leukemia inhibitory factor recept [symbol] | 92.44914 | 112 | 155.6189 |
| Q05940 | P07ef5 | solute carrier family 18 (vesicular | 374.4901 | 153 | 155.637 |
| Q9Y3Q5 | H18cd6 | putative DNA/chromatin binding [symbol] | 75.96908 | 150 | 155.6726 |
| Q15756 | H08ab7 | potassium inwardly-rectifying ch [symbol] | 149.0689 | 80.9 | 155.7504 |
| O75386 | M17cd3 | tubby like protein 3 | 128.6914 | 82.7 | 155.7811 |
| P50750 | D03ef5 | cyclin-dependent kinase 9 (CDC: | 270.8229 | 215 | 155.8317 |
| Q9NWY6 | A18gh3 | hypothetical protein FLJ20531 | 91.85185 | 130 | 156.1332 |
| P50391 | K05cd1 | pancreatic polypeptide receptor | 136.5278 | 112 | 156.1953 |
| Q9NX07 | L20gh6 | tRNA selenocysteine associated | 258.434 | 287 | 156.2449 |
| O75354 | A15ab4 | ectonucleoside triphosphate dip [symbol] | 386.4989 | 258 | 156.4638 |
| Q9H832 | L12gh5 | hypothetical protein FLJ13855 | 173.4437 | 307 | 156.9292 |
| Q9UBW8 | F18ef1 | COP9 constitutive photomorpho [symbol] | 102.8052 | 111 | 157.0119 |
| P14136 | E23ab5 | glial fibrillary acidic protein | 108.166 | 86.6 | 157.2761 |
| Q9NYF3 | F08ef1 | chromosome 5 open reading frar | 121.4116 | 94.3 | 157.5806 |
| Q15056 | A09gh2 | Williams-Beuren syndrome chror | 202.4969 | 124 | 157.6763 |
| O15534 | I16ab8 | period homolog 1 (Drosophila) | 237.2007 | 143 | 157.7195 |
| Q9Y399 | O21ef2 | mitochondrial ribosomal protein [symbol] | 229.9134 | 139 | 157.7339 |
| O14972 | A04cd6 | Down syndrome critical region g [symbol] | 230.0837 | 267 | 157.7386 |
| Q14113 | M15ab2 | AE binding protein 1 | 124.9796 | 95.3 | 157.955 |
| O95755 | O18cd5 | RAB36; member RAS oncogene | 132.9919 | 97 | 158.0474 |
| Q9NPC3 | I09gh4 | enhancer of invasion 10 | 104.2128 | 121 | 158.1023 |
| Q9Y6E9 | N24cd7 | sirtuin silent mating type informal | 111.8557 | 153 | 158.2227 |
| Q9Y5B8 | D17cd8 | NME7 | 92.69165 | 116 | 158.2444 |
| P49642 | I21ab8 | primase; polypeptide 1 (49 kD) | 135.2376 | 70.2 | 158.2887 |
| O60660 | J04ab2 | ash2 (absent; small; or homeotic | 274.3502 | 177 | 158.3372 |
| Q9H251 | O08gh7 | cadherin related 23 | 113.1038 | 184 | 158.4524 |
| Q9Y6C2 | A22cd7 | elastin microfibril interface locate | 107.9751 | 92.2 | 158.6648 |
| O95825 | D13cd5 | crystallin; zeta (quinone reducta [symbol] | 144.0183 | 133 | 158.6719 |
| Q9UHG3 | J02ef2 | prenylcysteine lyase | 250.3969 | 207 | 158.8419 |
| O00629 | J18ab6 | karyopherin alpha 4 (importin alp | 518.8184 | 295 | 158.8458 |
| Q15397 | B14gh1 | KIAA0020 gene product | 109.8533 | 134 | 158.9595 |
| Q14999 | H01gh1 | KIAA0076 gene product | 71.35119 | 173 | 159.0037 |
| Q12772 | M18cd2 | sterol regulatory element binding | 158.6108 | 94.6 | 159.1986 |
| O75150 | F09gh1 | ring finger protein 40 | 129.2351 | 169 | 159.2038 |
| O43423 | L07gh6 | acidic (leucine-rich) nuclear pho [symbol] | 140.3006 | 143 | 159.4372 |
| O43311 | D12cd8 | muscleblind-like (Drosophila) | 165.2243 | 99.4 | 159.4681 |
| Q9NYX4 | B07ef4 | calcyon; D1 dopamine receptor- [symbol] | 207.2791 | 147 | 159.5302 |
| Q9HD71 | G04gh4 | hypothetical nuclear factor SBB [symbol] | 123.7874 | 140 | 159.5491 |
| Q9NZH2 | N03cd8 | replication initiation region prote [symbol] | 225.0949 | 179 | 159.6113 |
| Q9NVH1 | B05gh3 | hypothetical protein FLJ10737 | 137.4673 | 217 | 159.6304 |
| Q9NVG3 | B11gh3 | hypothetical protein FLJ10751 | 96.28296 | 105 | 159.6829 |
| P52179 | A21cd4 | myomesin 1 (skelemin) (185 kD) | 157.0608 | 94 | 159.8272 |
| O94815 | B09cd3 | frizzled homolog 1 (Drosophila) | 93.97877 | 101 | 159.9198 |
| Q9UBY9 | G01ef4 | heat shock 27 kD protein family; [symbol] | 98.97552 | 102 | 160.1725 |
| Q9NVX7 | G18gh3 | hypothetical protein FLJ10450 | 111.5196 | 121 | 160.2507 |
| O95704 | A02cd6 | FE65-like protein 2 | 99.35088 | 115 | 160.3239 |
| P19801 | A23ab2 | amiloride binding protein 1 (amin | 164.6296 | 68.3 | 160.3477 |
| O43709 | L18ef3 | Williams Beuren syndrome chror | 127.8538 | 134 | 160.4415 |
| Q12839 | N24ef1 | H326 | 109.677 | 116 | 160.6141 |
| O43503 | D10ab8 | RAD51 homolog C (S. cerevisiae | 96.34779 | 97.6 | 160.6551 |
| Q07898 | J10cd4 | CD163 antigen | 87.73563 | 177 | 160.8614 |
| O96015 | F22cd5 | dynein; axonemal; light polypepti [symbol] | 94.54055 | 251 | 160.9963 |
| Q92524 | L11ab8 | proteasome (prosome; macropal [symbol] | 396.6515 | 220 | 161.0391 |
| Q92692 | B06ab8 | poliovirus receptor-related 2 (her | 302.7185 | 317 | 161.2569 |
| Q9Y6Y8 | I16cd7 | Sec23-interacting protein p125 | 104.2429 | 131 | 161.2684 |
| Q9NZ78 | J16gh3 | uncharacterized bone marrow pn [symbol] | 107.2001 | 127 | 161.3587 |
| Q15120 | G15cd1 | pyruvate dehydrogenase kinase; | 96.64061 | 107 | 161.6468 |
| P35241 | F22ab8 | radixin | 276.6012 | 213 | 161.7003 |
| Q9Y326 | D23ef2 | brain specific protein | 359.3582 | 160 | 161.7123 |
| Q92600 | J03cd5 | RCD1 required for cell differentia [symbol] | 115.6633 | 145 | 161.7142 |
| O00487 | N14cd5 | 26S proteasome-associated pad [symbol] | 329.3518 | 275 | 161.8834 |
| Q9Y689 | P21cd7 | ADP-ribosylation factor-like 5 | 137.7737 | 159 | 161.8965 |
| Q9ULB8 | P05cd8 | CMP-NeuAC: (beta)-N-acetylgala | 91.01128 | 121 | 161.9148 |
| O60897 | L09cd7 | RAB; member of RAS oncogene | 89.32474 | 112 | 162.0127 |
| P51809 | J05cd2 | synaptobrevin-like 1 | 228.3546 | 141 | 162.1052 |
| O75581 | A03ab7 | low density lipoprotein receptor-r | 122.4708 | 125 | 162.1159 |
| O43795 | F12ef4 | myosin class I; myh-1c | 259.4543 | 282 | 162.137 |
| Q14188 | H17ef5 | transcription factor Dp-2 (E2F dir | 107.308 | 204 | 162.1489 |
| P06280 | B07ab3 | galactosidase; alpha | 178.0238 | 304 | 162.2871 |
| Q14541 | O17ab7 | hepatocyte nuclear factor 4; gam | 549.8997 | 283 | 162.4659 |
| P57723 | E17gh8 | poly(rC) binding protein 4 | 186.2845 | 231 | 162.5267 |
| Q9NWU3 | A24gh3 | DEAD/H (Asp-Glu-Ala-Asp/His) t [symbol] | 154.71 | 104 | 162.6529 |
| Q9NPC5 | J16ef8 | nudix (nucleoside diphosphate [symbol] | 107.1863 | 98.4 | 162.6929 |
| Q9Y3C5 | B19ef3 | ring finger protein 11 | 160.0631 | 164 | 162.7518 |
| Q9NWX3 | B01gh2 | chromosome 20 open reading fr [symbol] | 98.58482 | 95.6 | 162.7744 |
| P52742 | I22cd3 | zinc finger portein 135 (clone pH | 81.4141 | 121 | 162.8382 |
| Q9Y313 | D17ef2 | PTD013 protein | 90.25232 | 116 | 163.4053 |
| Q9P2W9 | B11ef4 | syntaxin 18 | 128.6426 | 188 | 163.4277 |
| P14778 | F09ab6 | interleukin 1 receptor; type I | 264.5401 | 539 | 163.4432 |
| Q15464 | I12ef5 | SHB (Src homology 2 domain co [symbol] | 284.291 | 301 | 163.5379 |
| O15444 | H07cd2 | small inducible cytokine subfamil [symbol] | 244.6426 | 97.1 | 163.6053 |
| P49796 | P14ef4 | regulator of G-protein signalling: | 220.2769 | 99.3 | 163.6222 |
| Q9NUS7 | B04gh3 | hypothetical protein FLJ11164 | 81.76063 | 114 | 163.6422 |
| P48651 | L11ef3 | phosphatidylserine synthase 1 | 211.8695 | 139 | 163.6792 |
| Q07706 | F15cd4 | tetracycline transporter-like prote [symbol] | 143.0791 | 144 | 163.7206 |
| O75143 | B11gh1 | KIAA0652 gene product | 106.5331 | 155 | 163.7687 |
| O75039 | L19gh1 | KIAA0451 gene product | 174.142 | 135 | 163.8572 |
| P02261 | M10gh6 | H2A histone family; member N | 93.48591 | 273 | 163.8621 |
| Q9H9A0 | L20gh5 | hypothetical protein FLJ12895 | 86.42599 | 130 | 164.3655 |
| Q9NV35 | J21gh3 | hypothetical protein FLJ10958 | 99.15342 | 154 | 164.4852 |
| Q9P1G8 | C05gh4 | WW domain-containing adapter [symbol] | 119.4436 | 182 | 164.5311 |
| P26368 | D11cd7 | U2 small nuclear ribonucleoprote [symbol] | 217.0564 | 107 | 164.8861 |
| Q9BSZ7 | E11gh8 | DEAD/H (Asp-Glu-Ala-Asp/His) t [symbol] | 130.7858 | 111 | 164.9084 |
| Q9Y391 | G23ef2 | androgen-regulated short-chain [symbol] | 222.3497 | 128 | 164.9823 |
| Q9NYR5 | E05ef3 | HSPC126 protein | 99.37243 | 110 | 165.2098 |
| P05165 | N12ab7 | propionyl Coenzyme A carboxyla [symbol] | 100.892 | 120 | 165.6902 |
| Q9NPF4 | O21gh3 | O-sialoglycoprotein endopeptida [symbol] | 103.8223 | 112 | 165.9716 |
| P16106 | D01cd3 | H3 histone family; member K | 99.19006 | 130 | 166.1216 |
| P35249 | O03ef5 | replication factor C (activator 1) [symbol] | 105.2406 | 98.7 | 166.2092 |
| P28702 | P18gh1 | retinoid X receptor; beta | 74.914 | 142 | 166.2214 |
| P49674 | K14ab4 | casein kinase 1; epsilon | 99.33956 | 145 | 166.3564 |
| Q9NXW2 | I03gh2 | DnaJ (Hsp40) homolog; subfamil [symbol] | 247.7907 | 235 | 166.4412 |
| O14672 | I01ab2 | a disintegrin and metalloproteina [symbol] | 378.0042 | 226 | 166.5417 |
| Q9Y5B9 | I20cd7 | chromatin-specific transcription [symbol] | 163.0543 | 188 | 166.6045 |
| Q9H9J2 | H22gh5 | mitochondrial ribosomal protein [symbol] | 206.428 | 256 | 166.8142 |
| P23759 | G06ab8 | paired box gene 7 | 92.41833 | 200 | 166.8514 |
| Q16643 | L05ab4 | drebrin 1 | 265.008 | 152 | 166.9734 |
| P48454 | I21cd1 | protein phosphatase 3 (formerly: | 105.9496 | 169 | 167.0428 |
| Q9Y508 | F21ef4 | zinc finger protein 313 | 331.595 | 395 | 167.2366 |
| P19474 | G09cd2 | Sjogren syndrome antigen A1 (5: | 301.1776 | 183 | 167.414 |
| P46109 | N13ab5 | v-crk sarcoma virus CT10 oncog [symbol] | 151.2065 | 141 | 167.7716 |
| P54855 | B19cd4 | UDP glycosyltransferase 2 family | 137.8939 | 127 | 167.8346 |
| Q14242 | H14cd1 | selectin P ligand | 112.9679 | 130 | 167.9184 |
| Q9BSS1 | P17gh8 | hypothetical protein FLJ14525 | 86.75908 | 113 | 167.974 |
| Q9Y3B2 | L23ef1 | homolog of yeast exosomal core | 152.0751 | 140 | 167.9915 |
| P31391 | N03cd1 | somatostatin receptor 4 | 147.3942 | 92.5 | 168.0254 |
| O60623 | O03ab4 | diptheria toxin resistance protein | 210.9658 | 170 | 168.0762 |
| Q99959 | L24ab8 | plakophilin 2 | 362.9423 | 211 | 168.1655 |
| | B21gh7 | leukocyte immunoglobulin-like re [symbol] | 139.2571 | 234 | 168.1829 |
| Q01658 | P18ab5 | down-regulator of transcription 1: | 205.2249 | 122 | 168.2606 |
| Q9HCS4 | P10gh7 | HMG-box transcription factor TCI | 76.48423 | 156 | 168.2926 |
| Q9P035 | N20ef2 | butyrate-induced transcript 1 | 102.716 | 142 | 168.3034 |
| Q9C0K7 | P24gh3 | amyotrophic lateral sclerosis 2 ( [symbol] | 102.352 | 108 | 168.4355 |
| Q9NYF4 | I13ef3 | chromosome 5 open reading frar [symbol] | 89.72908 | 137 | 168.4424 |
| P30042 | M19cd4 | chromosome 21 open reading fra [symbol] | 98.84241 | 97.5 | 168.5171 |
| O75325 | K16cd6 | glioma amplified on chromosome | 116.3239 | 83.3 | 168.5172 |
| Q9Y670 | N01ef2 | transient receptor potential catio [symbol] | 96.41826 | 90.8 | 168.5297 |
| Q9UPR8 | I14ef8 | KIAA1084 protein | 101.3235 | 111 | 168.5336 |
| Q9Y2X7 | I21ef4 | G protein-coupled receptor kinas | 160.7403 | 167 | 168.7352 |
| P47872 | D24cd1 | secretin receptor | 174.6672 | 209 | 168.738 |
| O43353 | C17cd4 | receptor-interacting serine-threo [symbol] | 272.5279 | 388 | 168.7722 |
| P32321 | P14ab5 | dCMP deaminase | 218.5685 | 60.1 | 168.8404 |
| O43612 | J16ab4 | hypocretin (orexin) neuropeptide | 66.45691 | 194 | 168.9064 |
| Q92874 | M15ab4 | deoxyribonuclease I-like 2 | 218.1787 | 146 | 168.9596 |
| Q9UI90 | I15ef8 | PRO0038 protein | 89.17218 | 389 | 168.9849 |
| P30519 | N05ab6 | heme oxygenase (decycling) 2 | 586.3897 | 387 | 169.1269 |
| Q9NVE7 | D03gh3 | hypothetical protein FLJ10782 | 141.6695 | 148 | 169.2644 |
| Q9P010 | A14ef8 | HSPC154 protein | 227.9143 | 246 | 169.4393 |
| Q9UH59 | M04cd8 | bromodomain-containing 7 | 147.6635 | 103 | 169.5045 |
| Q9UM82 | E01cd6 | spermatogenesis associated 2 | 230.7114 | 221 | 169.6678 |
| Q9P2Y4 | N14ef1 | zinc finger protein 219 | 105.6753 | 175 | 169.7666 |
| Q9GZS3 | H19gh7 | recombination protein REC14 | 124.7245 | 162 | 169.9984 |
| P07686 | J17ab3 | hexosaminidase B (beta polypep [symbol] | 217.0423 | 135 | 170.0092 |
| Q9NZV6 | L06ef2 | selenoprotein X; 1 | 241.3633 | 202 | 170.0232 |
| O14678 | E17cd1 | ATP-binding cassette; sub-family | 77.65216 | 127 | 170.0525 |
| P29972 | O04ab2 | aquaporin 1 (channel-forming int [symbol] | 309.1218 | 134 | 170.2078 |
| O95373 | D03cd6 | RAN binding protein 7 | 247.4368 | 113 | 170.45 |
| Q15436 | O12cd6 | Sec23 homolog A (S. cerevisiae) | 90.05071 | 100 | 170.9386 |
| Q9BQ67 | K23gh6 | glutamate rich WD repeat protein | 140.3198 | 112 | 170.966 |
| P35321 | D21gh6 | small proline-rich protein 1A | 62.28271 | 237 | 171.0104 |
| Q9UPQ8 | I10ef8 | KIAA1094 protein | 76.76245 | 123 | 171.0467 |
| O43292 | A19cd4 | GPAA1P anchor attachment prot [symbol] | 95.36294 | 92.4 | 171.0704 |
| Q9UBM3 | G02cd1 | proteasome (prosome; macropai [symbol] | 287.6026 | 204 | 171.1554 |
| P19338 | M20ab7 | nucleolin | 148.8642 | 87.6 | 171.2094 |
| Q9NPE6 | E17gh1 | sperm associated antigen 4 | 111.172 | 135 | 171.4356 |
| O00442 | J12cd3 | RTC domain containing 1 | 274.6701 | 279 | 171.531 |
| P28356 | D09ef1 | homeo box D9 | 84.5638 | 106 | 171.5949 |
| P49447 | G06ef1 | cytochrome b-561 | 174.7152 | 167 | 171.6447 |
| Q9UQA4 | P18cd4 | nuclear factor (erythroid-derived | 227.4253 | 470 | 171.8945 |
| O15254 | D09ab2 | acyl-Coenzyme A oxidase 3; pris [symbol] | 192.2301 | 192 | 172.0218 |
| Q9NWW6 | B11gh2 | hypothetical protein FLJ20559 | 198.7861 | 220 | 172.0261 |
| O75391 | M08cd5 | sperm associated antigen 7 | 154.1159 | 140 | 172.0658 |
| Q92959 | H19cd2 | solute carrier family 21 (prostag [symbol] | 86.25485 | 102 | 172.112 |
| O00499 | G06ab2 | bridging integrator 1 | 162.8584 | 111 | 172.4453 |
| Q14197 | J05ab6 | immature colon carcinoma trans [symbol] | 90.92583 | 102 | 172.5526 |
| Q9NXS9 | K15gh2 | hypothetical protein FLJ20071 | 121.4575 | 103 | 172.5552 |
| O75954 | N18cd6 | tetraspan transmembrane 4 sup [symbol] | 83.06795 | 119 | 172.5557 |
| Q9UJY9 | E17cd8 | lifeguard | 156.5909 | 75.6 | 172.6495 |
| P82912 | F10gh5 | mitochondrial ribosomal protein [symbol] | 105.0578 | 196 | 172.7091 |
| Q12918 | J02ab6 | killer cell lectin-like receptor sub [symbol] | 205.6225 | 290 | 172.8141 |
| Q14789 | E01ab6 | golgi autoantigen; golgin subfam [symbol] | 96.09162 | 194 | 173.0059 |
| Q04206 | I23ef6 | v-rel reticuloendotheliosis viral o [symbol] | 328.9295 | 242 | 173.0443 |
| P19838 | M15ef5 | nuclear factor of kappa light poly [symbol] | 229.2262 | 277 | 173.0997 |
| Q92748 | A24cd2 | thyroid hormone responsive (SP [symbol] | 101.3906 | 88.2 | 173.314 |
| O95095 | P13ab2 | amyloid beta precursor protein ( [symbol] | 199.951 | 201 | 173.3314 |
| P52209 | K18ab8 | phosphogluconate dehydrogena [symbol] | 211.8178 | 147 | 173.42 |
| P42566 | H10ef5 | epidermal growth factor receptor | 194.9437 | 158 | 173.4643 |
| Q13772 | D05gh6 | nuclear receptor coactivator 4 | 273.5167 | 367 | 173.5038 |
| P43897 | D09gh6 | Ts translation elongation factor; [symbol] | 325.7575 | 226 | 173.8489 |
| P09329 | D08ef7 | phosphoribosyl pyrophosphate [symbol] | 103.7882 | 86.2 | 174.5738 |
| O75618 | F24cd4 | death effector domain-containing | 172.0163 | 176 | 174.7689 |
| Q9UHI9 | B18ef1 | flavohemoprotein b5 + b5R | 169.2028 | 197 | 174.7919 |
| Q9H824 | J07gh6 | F-box only protein 22 | 89.2751 | 161 | 175.62 |
| Q9UQ43 | G07ab3 | BAI1-associated protein 2 | 184.8198 | 165 | 175.6399 |
| O43173 | N08ef1 | sialyltransferase 8C (alpha2; 3Ga [symbol] | 220.9909 | 256 | 175.9869 |
| Q9BZZ2 | G13gh7 | sialoadhesin | 147.52 | 189 | 175.9952 |
| Q92611 | I16gh1 | KIAA0212 gene product | 96.3144 | 94.3 | 176.0348 |
| Q969V6 | K14gh7 | megakaryoblastic leukemia (tran [symbol] | 135.6533 | 161 | 176.0531 |
| P08107 | K14ab7 | heat shock 70 kD protein 1B | 153.5141 | 106 | 176.1145 |
| Q9BQ24 | E21gh6 | hypothetical protein MGC2550 | 153.9741 | 255 | 176.3889 |
| Q9H7X1 | N07gh5 | hypothetical protein FLJ14153 | 155.291 | 236 | 176.5938 |
| Q99616 | D09cd2 | small inducible cytokine subfamil [symbol] | 130.1641 | 100 | 176.5958 |
| Q92896 | L04cd7 | golgi apparatus protein 1 | 97.73429 | 90.3 | 176.622 |
| Q14582 | J16ef6 | Mad4 homolog | 210.3185 | 244 | 176.6383 |
| Q13505 | K21ab7 | metaxin 1 | 199.1231 | 168 | 176.6745 |
| Q13722 | O24cd8 | chromosome 3p21.1 gene seque [symbol] | 85.85963 | 144 | 176.7101 |
| P01308 | I10ab6 | insulin | 367.022 | 113 | 176.7437 |
| O00405 | D19ab8 | protein phosphatase 1; regulator | 233.0866 | 306 | 176.8763 |
| P41215 | E03ab5 | fatty-acid-Coenzyme A ligase; lo [symbol] | 267.9822 | 199 | 177.1301 |
| P31314 | F04ef6 | homeo box 11 (T-cell lymphoma | 107.4127 | 170 | 177.4236 |
| O95427 | G21cd8 | phosphatidylinositol glycan; clas [symbol] | 84.22817 | 116 | 177.4629 |
| Q12981 | C24ef6 | BCL2/adenovirus E1B 19 kD inte [symbol] | 82.38214 | 99.8 | 177.5246 |
| Q9Y5V1 | L10ef1 | HSPC042 protein | 212.074 | 184 | 177.7635 |
| Q9H4B4 | O22ab4 | cytokine-inducible kinase | 161.982 | 194 | 177.788 |
| Q9H6D1 | P15gh5 | chromosome 20 open reading fr [symbol] | 95.43432 | 108 | 177.8526 |
| O60504 | J18cd5 | vinexin beta (SH3-containing ad [symbol] | 109.3831 | 99.8 | 177.8876 |
| Q9UNK0 | I02cd5 | syntaxin 8 | 218.2149 | 171 | 177.9777 |
| O95657 | B24cd4 | proline-serine-threonine phosph [symbol] | 369.9485 | 158 | 178.0072 |
| P20645 | C05ab7 | mannose-6-phosphate receptor ( [symbol] | 355.8249 | 234 | 178.1318 |
| O14569 | B03ab2 | putative tumor suppressor | 122.4884 | 79.2 | 178.136 |
| O15293 | D18cd3 | MAP-kinase activating death don [symbol] | 157.092 | 114 | 178.1677 |
| P00390 | E14ef7 | glutathione reductase | 282.1492 | 196 | 178.1741 |
| Q9HBV3 | K24gh5 | PP3111 protein | 229.2714 | 361 | 178.4093 |
| Q14558 | H17ab8 | phosphoribosyl pyrophosphate [symbol] | 301.219 | 227 | 178.4567 |
| O14710 | F01ef5 | cell cycle progression 2 protein | 261.2178 | 189 | 178.7923 |
| Q9UJ70 | J10ef3 | N-acetylglucosamine kinase | 151.2986 | 276 | 178.7992 |
| Q13397 | M18cd6 | zinc finger protein 238 | 215.883 | 285 | 178.9318 |
| Q9NRG4 | B21gh4 | HSKM-B protein | 143.1326 | 279 | 178.999 |
| P51449 | B01cd2 | RAR-related orphan receptor C | 236.6977 | 97.2 | 179.0372 |
| Q9H768 | O21gh7 | synaptotagmin-like 2 | 83.78255 | 121 | 179.0813 |
| Q14332 | F22ef5 | frizzled homolog 2 (Drosophila) | 86.25011 | 129 | 179.1323 |
| Q13207 | N13cd2 | T-box 2 | 118.9378 | 108 | 179.1858 |
| Q9H6B8 | B19gh5 | hypothetical protein similar to m [symbol] | 134.338 | 129 | 179.5015 |
| O00462 | J09ab7 | mannosidase; beta A; lysosomal | 83.23817 | 107 | 179.6953 |
| Q9Y3D8 | H14ef2 | adrenal gland protein AD-004 | 178.4931 | 163 | 179.7989 |
| P17900 | F19ab3 | GM2 ganglioside activator protei [symbol] | 124.7747 | 232 | 179.914 |
| Q9P0M4 | O10cd8 | interleukin 17C | 260.8753 | 223 | 180.0241 |
| | F13gh8 | G protein-coupled receptor 54 | 87.92591 | 133 | 180.0735 |
| Q9NZ92 | J02gh3 | SWI/SNF related; matrix associa [symbol] | 267.7307 | 247 | 180.206 |
| Q9NX94 | L18gh6 | hypothetical protein FLJ20154 | 133.6318 | 165 | 180.3424 |
| P04049 | G12ef5 | v-raf-1 murine leukemia viral onc [symbol] | 225.086 | 183 | 180.3925 |
| Q13181 | B06cd1 | ATP-binding cassette; sub-family | 167.9383 | 127 | 180.4651 |
| P22102 | M05ab6 | phosphoribosylglycinamide form [symbol] | 295.0489 | 180 | 180.5026 |
| Q9NQZ8 | A09gh5 | endothelial zinc finger protein inc [symbol] | 96.79284 | 138 | 180.7283 |
| Q9UN30 | D04cd2 | sex comb on midleg-like 1 (Dros [symbol] | 97.47885 | 86 | 181.0121 |
| P26651 | J04ef6 | zinc finger protein 36; C3H type; | 286.0733 | 162 | 181.0638 |
| P18887 | O06ef6 | X-ray repair complementing defe [symbol] | 85.61601 | 102 | 181.0778 |
| Q9Y2S0 | P14ef1 | RNA polymerase I 16 kDa subun [symbol] | 182.9338 | 149 | 181.2184 |
| P49023 | I05ef7 | paxillin | 313.7788 | 334 | 181.2995 |
| P49321 | O03ab7 | nuclear autoantigenic sperm pro [symbol] | 119.903 | 78.2 | 181.3524 |
| Q13428 | M14cd1 | Treacher Collins-Franceschetti s [symbol] | 164.3621 | 123 | 181.4282 |
| Q9HCS6 | G07gh4 | vacuolar protein sorting 11 (yeas [symbol] | 177.2833 | 302 | 181.4601 |
| P20813 | I02gh6 | cytochrome P450; subfamily IIB [symbol] | 122.3562 | 162 | 181.476 |
| O00459 | C05cd1 | phosphoinositide-3-kinase; regul [symbol] | 145.3741 | 160 | 181.534 |
| P50151 | H24ab4 | guanine nucleotide binding prote [symbol] | 186.5329 | 193 | 182.0963 |
| Q16850 | D12ab3 | cytochrome P450; 51 (lanosterol | 353.9667 | 116 | 182.1309 |
| Q9ULW3 | J09cd8 | TATA-binding protein-binding pr [symbol] | 97.23454 | 207 | 182.1565 |
| Q16378 | O20cd7 | proline rich 4 (lacrimal) | 81.91 | 152 | 182.3589 |
| P41240 | J14ab4 | c-src tyrosine kinase | 175.1285 | 153 | 182.4257 |
| Q16649 | G05cd1 | nuclear factor; interleukin 3 regul [symbol] | 353.6698 | 169 | 182.4266 |
| Q9Y3M2 | J03ef8 | chromosome 22 open reading fr [symbol] | 74.27488 | 194 | 182.6022 |
| Q9UK58 | M14gh4 | cyclin L ania-6a | 191.3619 | 313 | 182.6351 |
| P47874 | M23cd1 | olfactory marker protein | 250.3691 | 172 | 182.73 |
| O95620 | O03cd7 | protein similar to E. coli yhdg and | 227.8932 | 207 | 183.1055 |
| Q9UHV2 | J11cd8 | CDK4-binding protein p34SEI1 | 144.2806 | 163 | 183.5211 |
| P02810 | F07gh6 | proline-rich protein HaeIII subfa [symbol] | 410.3478 | 392 | 183.6977 |
| O94903 | K04cd7 | proline synthetase co-transcribe [symbol] | 242.2062 | 146 | 183.9744 |
| Q9P0W6 | I03ef4 | lipopolysaccharide specific resp [symbol] | 149.1418 | 177 | 184.0725 |
| Q9H8X4 | O15gh5 | stromal membrane-associated pr [symbol] | 358.1542 | 342 | 184.0879 |
| P02786 | D24ef5 | transferrin receptor (p90; CD71) | 320.8365 | 257 | 184.2545 |
| | E15ef1 | immunoglobulin heavy constant [symbol] | 386.489 | 176 | 184.3533 |
| Q99717 | J01ab7 | MAD; mothers against decapent [symbol] | 227.5817 | 153 | 184.3978 |
| Q15036 | B23gh1 | sorting nexin 17. | 303.8909 | 411 | 184.4831 |
| P31751 | A08ab2 | v-akt murine thymoma viral onco [symbol] | 253.7531 | 253 | 184.8245 |
| P11177 | G21ab8 | pyruvate dehydrogenase (lipoam [symbol] | 229.1486 | 150 | 185.0719 |
| Q9UEQ6 | F22ab2 | arrestin; beta 2 | 190.7331 | 128 | 185.1221 |
| Q16531 | J11ab5 | damage-specific DNA binding pr [symbol] | 205.1934 | 229 | 185.1363 |
| P28698 | G24cd3 | zinc finger protein 42 (myeloid-s [symbol] | 76.12612 | 105 | 185.3347 |
| P41968 | N12ef4 | melanocortin 3 receptor | 225.3987 | 287 | 185.5363 |
| P54920 | E03cd4 | N-ethylmaleimide-sensitive facto [symbol] | 306.753 | 267 | 185.697 |
| Q99487 | C19ab8 | platelet-activating factor acetylhy [symbol] | 228.4138 | 241 | 185.7091 |
| Q9H0C8 | N15gh7 | integrin-linked kinase-associated | 120.8459 | 109 | 185.7532 |
| P17252 | F17ab8 | protein kinase C; alpha | 108.3851 | 74 | 185.8466 |
| P06493 | B01ef5 | cell division cycle 2; G1 to S and | 108.1696 | 76.2 | 185.8754 |
| O00139 | C14ab7 | kinesin heavy chain member 2 | 140.4141 | 205 | 185.9437 |
| P54259 | N05ef6 | dentatorubral-pallidoluysian atro [symbol] | 165.5295 | 152 | 186.0962 |
| Q99757 | G10cd8 | thioredoxin 2 | 309.0415 | 163 | 186.106 |
| Q9NW68 | B12gh2 | hypothetical protein FLJ10276 | 107.4044 | 242 | 186.1575 |
| P16106 | O16gh6 | H3 histone family; member J | 137.9914 | 135 | 186.2589 |
| P40692 | M06ef6 | mutL homolog 1; colon cancer; n | 98.77988 | 108 | 186.2872 |
| Q99798 | E01ab2 | aconilase 2; mitochondrial | 615.211 | 291 | 186.5524 |
| P53618 | A11ef3 | coatomer protein complex; subu [symbol] | 238.4951 | 102 | 186.6268 |
| Q9UJ69 | N24gh2 | Toll-interacting protein | 211.4055 | 259 | 186.6408 |
| Q9NRM2 | P22ef8 | zinc finger protein 277 | 232.9567 | 227 | 186.8854 |
| Q92879 | B06cd6 | CUG triplet repeat; RNA binding | 217.3752 | 198 | 187.122 |
| O75399 | H22gh1 | deformed epidermal autoregulat [symbol] | 189.8387 | 266 | 187.2872 |
| Q9BZE4 | I17cd8 | G protein-binding protein CRFG | 151.2547 | 214 | 187.3094 |
| Q9NXB2 | G08gh2 | hypothetical protein FLJ20343 | 259.0672 | 285 | 187.4246 |
| O95424 | M06ef4 | MYLE protein | 103.2077 | 184 | 187.5574 |
| P54577 | D14cd3 | tyrosyl-tRNA synthetase | 145.5748 | 108 | 187.5756 |
| O43692 | N03ef7 | protease inhibitor 15 | 102.5299 | 163 | 187.7565 |
| O60507 | J03cd3 | tyrosylprotein sulfotransferase 1 | 190.271 | 136 | 188.0843 |
| Q9BYS9 | B15gh5 | MMS19-like (MET18 homolog; S. | 99.29429 | 117 | 188.1006 |
| P32780 | G11ab6 | general transcription factor IIH; p [symbol] | 258.9396 | 180 | 188.3175 |
| Q14004 | D07ef5 | cell division cycle 2-like 5 (cholin | 92.56943 | 158 | 188.3656 |
| Q15910 | L08ab5 | enhancer of zeste homolog 2 (D [symbol] | 213.1801 | 306 | 188.8314 |
| P47974 | H22ab4 | zinc finger protein 36; C3H type- [symbol] | 187.7102 | 163 | 188.8801 |
| Q13177 | E20ab8 | p21 (CDKN1A)-activated kinase | 367.7706 | 232 | 188.9048 |
| | P16gh8 | baculoviral IAP repeat-containin [symbol] | 251.3679 | 126 | 189.0052 |
| P98172 | J01ef7 | ephrin-B1 | 122.8828 | 109 | 189.5198 |
| Q9BQB1 | N04gh6 | hypothetical protein FLJ10342 | 113.906 | 172 | 189.7648 |
| O75472 | H11cd5 | DnaJ (Hsp40) homolog; subfamil [symbol] | 227.1041 | 204 | 189.9354 |
| O75608 | K06cd6 | lysophospholipase I | 357.6112 | 374 | 190.1833 |
| O00193 | E14ef3 | small acidic protein | 223.9793 | 284 | 190.2397 |
| Q14452 | B08ab5 | glucocorticoid receptor DNA bin [symbol] | 158.8451 | 147 | 190.439 |
| P55268 | P18ef6 | laminin; beta 2 (laminin S) | 125.0717 | 152 | 190.5787 |
| Q9P006 | A20ef8 | mitochondrial ribosomal protein [symbol] | 114.6952 | 151 | 190.6578 |
| Q9Y365 | P07ef1 | CGI-111 protein | 158.6983 | 176 | 190.6804 |
| Q9NQ34 | A10gh4 | chromosome 11 open reading fr [symbol] | 144.3438 | 161 | 190.6944 |
| Q9NWY4 | O14gh2 | hypothetical protein FLJ20534 | 70.02507 | 110 | 191.0561 |
| Q9UBS8 | P17ab2 | ring finger protein 14 | 227.9827 | 247 | 191.1542 |
| Q9UBS4 | J06ef2 | DnaJ (Hsp40) homolog; subfamil [symbol] | 181.8446 | 175 | 191.2168 |
| Q12797 | J22ab2 | aspartate beta-hydroxylase | 101.2251 | 86.5 | 191.3988 |
| Q9H4I9 | P10gh8 | hypothetical gene supported by [symbol] | 167.5049 | 219 | 191.4831 |
| O14907 | E24ef8 | Tax interaction protein 1 | 351.7729 | 403 | 191.7684 |
| Q14673 | B09gh1 | Bcl-2-associated transcription fa [symbol] | 97.16912 | 117 | 191.9499 |
| Q9NWB7 | P01gh2 | estrogen-related receptor beta li [symbol] | 567.7076 | 84.6 | 192.0982 |
| Q9BVH4 | G21gh6 | hypothetical protein MGC3196 | 164.8572 | 169 | 192.4133 |
| P22897 | I17ab7 | mannose receptor; C type 1 | 77.30638 | 100 | 192.4612 |
| P19012 | L04ab6 | keratin 15 | 146.5632 | 130 | 192.6097 |
| Q14738 | C16cd1 | protein phosphatase 2; regulator | 224.4642 | 183 | 192.7984 |
| O95152 | F04cd4 | vesicle trafficking protein | 197.6319 | 196 | 192.9314 |
| P78426 | A09cd3 | NK6 transcription factor homolog | 198.5291 | 353 | 193.0203 |
| Q14790 | P14ab3 | caspase 8; apoptosis-related cys | 124.8603 | 284 | 193.1013 |
| P07711 | N13ab4 | cathepsin L | 137.16 | 121 | 193.1454 |
| P32942 | B02ab6 | intercellular adhesion molecule 3 | 140.3673 | 140 | 193.1656 |
| | L18ab2 | ATPase; Na+/K+ transporting; a [symbol] | 188.9371 | 131 | 194.2503 |
| Q9NWS0 | F13gh2 | hypothetical protein FLJ20643 | 81.29108 | 107 | 194.4652 |
| Q9NWZ5 | O06gh2 | uridine kinase-like 1 | 176.188 | 358 | 194.4782 |
| O75792 | D15cd6 | ribonuclease HI; large subunit | 100.3515 | 99.6 | 194.705 |
| P05141 | I04ab2 | solute carrier family 25 (mitocho [symbol] | 454.9832 | 311 | 194.7577 |
| Q9NX38 | K22gh2 | hypothetical protein FLJ20457 | 140.7383 | 161 | 195.2668 |
| Q9Y682 | A18cd8 | sec22 homolog | 193.7656 | 271 | 195.3918 |
| P78381 | C02cd4 | solute carrier family 35 (UDP-gal [symbol] | 276.1892 | 207 | 195.6585 |
| Q9UMP5 | B23ef3 | replication initiation region protei [symbol] | 270.4297 | 179 | 195.6957 |
| Q9P0Q0 | C15ef3 | hypothetical protein HSPC226 | 129.6054 | 241 | 195.7376 |
| Q9UBQ3 | M12cd7 | polyamine-modulated factor 1 | 189.4397 | 172 | 195.7465 |
| P27540 | D22ab2 | aryl hydrocarbon receptor nuclea [symbol] | 239.4814 | 258 | 195.776 |
| Q9Y2W1 | F03cd5 | thyroid hormone receptor-associ [symbol] | 160.2008 | 248 | 195.7775 |
| P82267 | E15ab4 | adaptor-related protein complex | 198.6022 | 231 | 195.7793 |
| P00918 | C16ab3 | carbonic anhydrase II | 283.4506 | 172 | 195.9099 |
| Q9Y663 | E07cd6 | heparan sulfate (glucosamine) 3 [symbol] | 259.052 | 99.8 | 196.1045 |
| Q9NX40 | K18gh2 | ovarian carcinoma immunoreacti [symbol] | 119.6155 | 247 | 196.1836 |
| P50222 | L09ab7 | mesenchyme homeo box 2 (grow [symbol] | 165.3048 | 223 | 196.5502 |
| Q13610 | C16cd7 | nuclear phosphoprotein similar t [symbol] | 160.8948 | 331 | 196.7055 |
| P49591 | B12cd2 | seryl-tRNA synthetase | 152.5268 | 69.6 | 196.7944 |
| P04554 | C19gh5 | protamine 3 | 314.8934 | 296 | 196.9835 |
| P02538 | N12ef6 | keratin 6A | 227.8925 | 277 | 197.1759 |
| Q13637 | G23cd7 | RAB32; member RAS oncogene | 392.0158 | 269 | 197.2609 |
| P39086 | J02ab3 | glutamate receptor; ionotropic; k [symbol] | 184.3318 | 210 | 197.334 |
| Q9H8F9 | H20gh5 | hypothetical protein 24432 | 287.08 | 202 | 197.402 |
| Q99460 | L13ab8 | proteasome (prosome; macropa [symbol] | 298.3527 | 228 | 197.4025 |
| Q9UKP6 | D20gh1 | G protein-coupled receptor 14 | 213.3717 | 142 | 197.5131 |
| Q16589 | A04ef5 | cyclin G2 | 99.61434 | 214 | 197.5658 |
| O95316 | M03cd5 | ribosomal protein S6 kinase; 90 k | 144.8234 | 145 | 197.5662 |
| O14545 | A09cd7 | FLN29 gene product | 238.4292 | 288 | 197.8936 |
| Q9Y249 | P03cd6 | homologous to yeast nitrogon pe [symbol] | 191.1146 | 224 | 198.1049 |
| Q92794 | K12cd4 | zinc finger protein 220 | 134.123 | 179 | 198.312 |
| P36894 | A12ab3 | bone morphogenetic protein rece [symbol] | 149.2953 | 236 | 198.4963 |
| P79525 | L21ab7 | MHC class I polypeptide-related | 120.8289 | 125 | 198.5782 |
| O14947 | K04ab6 | laminin; beta 3 (nicein (125 kD); [symbol] | 360.8887 | 284 | 199.2636 |
| Q12899 | K16cd3 | tripartite motif-containing 26 | 188.6935 | 250 | 199.2998 |
| | M24gh8 | zinc finger protein 289; ID1 regul [symbol] | 91.37228 | 137 | 199.4969 |
| Q9UIJ5 | A18ef2 | zinc finger; DHHC domain contai [symbol] | 436.5048 | 452 | 199.696 |
| P46821 | J11ab7 | microtubule-associated protein 1 | 121.2356 | 136 | 199.8286 |
| O60906 | P04cd1 | sphingomyelin phosphodiesteras | 282.5771 | 281 | 199.835 |
| P04040 | O18ab5 | catalase | 160.7098 | 239 | 199.9283 |
| O60709 | B16cd5 | dynamin 1-like | 85.33527 | 220 | 199.9492 |
| Q14714 | A02cd4 | sarcospan (Kras oncogene-asso [symbol] | 378.724 | 148 | 200.1413 |
| P39687 | F01cd4 | acidic (leucine-rich) nuclear pho [symbol] | 226.8272 | 108 | 200.186 |
| O75341 | K16cd4 | BRCA1 associated protein | 151.895 | 209 | 200.2666 |
| Q9NUN1 | D16gh3 | hypothetical protein FLJ11259 | 152.5767 | 196 | 200.5435 |
| Q9UHR5 | L22ef7 | transcriptional regulator protein | 225.1172 | 345 | 200.7483 |
| Q9UMX3 | D15gh8 | BCL2-related ovarian killer | 185.2616 | 176 | 200.9857 |
| Q02297 | J05ef6 | neuregulin 1 | 89.51288 | 234 | 201.3025 |
| Q9BQ83 | A21gh6 | hypothetical protein MGC5178 | 302.7768 | 398 | 201.3498 |
| O43491 | C22ab4 | erythrocyte membrane protein b [symbol] | 265.5557 | 144 | 201.5208 |
| Q92922 | N16cd1 | SWI/SNF related; matrix associa [symbol] | 251.3134 | 195 | 201.571 |
| Q16099 | J11ef3 | glutamate receptor; ionotropic; k [symbol] | 200.0311 | 288 | 201.66 |
| O95328 | B06gh4 | hypothetical protein 628 | 253.2751 | 240 | 201.6989 |
| Q06265 | C15cd1 | polymyositis/scleroderma autoan | 167.3169 | 182 | 202.2097 |
| Q01780 | K12gh6 | polymyositis/scleroderma autoan | 196.9508 | 270 | 202.2308 |
| Q9NPF9 | G07ef3 | HeLa cyclin-dependent kinase 2 | 251.6953 | 170 | 202.5242 |
| Q9P1U0 | B03ef4 | zinc ribbon domain containing; 1 | 110.7474 | 151 | 202.7351 |
| Q13480 | C02ab5 | GRB2-associated binding protei [symbol] | 171.5634 | 150 | 203.065 |
| O95205 | O05gh1 | C3H-type zinc finger protein; sim [symbol] | 138.7838 | 267 | 203.3797 |
| O76091 | I11cd1 | nitrilase 1 | 194.24 | 179 | 203.4317 |
| Q9Y364 | F21ef2 | DKFZP434J154 protein | 208.15 | 322 | 203.5516 |
| P51116 | I14cd5 | fragile × mental retardation; auto | 215.9784 | 213 | 203.6388 |
| P49915 | H19cd4 | guanine monphosphate syntheta [symbol] | 175.5001 | 115 | 203.6534 |
| Q9UHR3 | O04cd8 | nasopharyngeal carcinoma susc [symbol] | 120.0173 | 196 | 203.8212 |
| Q15007 | O10cd5 | Wilms' tumour 1-associating prot [symbol] | 237.1077 | 258 | 204.228 |
| P24941 | B03ef5 | cyclin-dependent kinase 2 | 98.60804 | 214 | 204.3554 |
| P53634 | L01ef7 | cathepsin C | 97.3314 | 77.9 | 204.771 |
| Q13123 | K03ef7 | IK cytokine; down-regulator of H [symbol] | 108.6134 | 153 | 204.9565 |
| Q9NUT6 | P21gh3 | hypothetical protein FLJ11151 | 285.3106 | 565 | 205.0219 |
| Q9Y279 | B23cd7 | Ig superfamily protein | 356.8489 | 226 | 205.0366 |
| O75319 | H07cd3 | dual specificity phosphatase 11 [symbol] | 250.2151 | 205 | 205.1456 |
| Q9NYH9 | L08ef3 | hepatocellular carcinoma-associ [symbol] | 287.2586 | 283 | 205.223 |
| Q9UNF0 | M20cd7 | protein kinase C and casein kina [symbol] | 438.5503 | 349 | 205.53 |
| O60437 | D03ab8 | periplakin | 443.9882 | 301 | 205.5879 |
| Q9NZN8 | N22cd8 | CCR4-NOT transcription comple [symbol] | 189.665 | 224 | 205.673 |
| Q14232 | A18ab4 | eukaryotic translation initiation fa [symbol] | 273.7271 | 210 | 205.7436 |
| O00142 | C03ef5 | thymidine kinase 2; mitochondria | 124.7128 | 147 | 205.9951 |
| O60443 | N15ab5 | deafness; autosomal dominant 5 | 122.4035 | 264 | 206.3187 |
| O95406 | J20cd5 | cornichon-like | 181.7181 | 138 | 206.4442 |
| Q12841 | E24cd7 | follistatin-like 1 | 80.93058 | 97.7 | 206.5097 |
| O15217 | B04ab5 | glutathione S-transferase A4 | 78.38055 | 91.3 | 206.6118 |
| P10645 | E01ab4 | chromogranin A (parathyroid sec | 115.7467 | 283 | 206.8714 |
| Q9HA66 | D09gh5 | G protein beta subunit-like | 198.1992 | 192 | 206.9573 |
| Q9Y439 | B06ef8 | PRP31 pre-mRNA processing fa [symbol] | 175.7234 | 310 | 207.0904 |
| Q9Y386 | G08ef2 | CGI-78 protein | 325.6509 | 246 | 207.7702 |
| P53990 | D21gh1 | KIAA0174 gene product | 230.1187 | 401 | 207.9831 |
| O95865 | O11ef3 | dimethylarginine dimethylaminoh [symbol] | 125.0105 | 157 | 208.0048 |
| | B21ef1 | immunoglobulin lambda-like poly | 142.7367 | 230 | 208.1338 |
| Q15834 | I17cd7 | hepatitis delta antigen-interactin [symbol] | 320.7647 | 226 | 208.1753 |
| P18846 | H05ab4 | activating transcription factor 1 | 470.9776 | 206 | 208.3404 |
| Q14349 | A23cd7 | HLA-B associated transcript 8 | 138.8938 | 175 | 208.5984 |
| Q9NVM5 | M08gh3 | hypothetical protein FLJ10637 | 98.19923 | 115 | 209.0315 |
| P12955 | N18ab7 | peptidase D | 133.8157 | 192 | 209.0325 |
| Q99700 | D14cd1 | spinocerebellar ataxia 2 (olivopo [symbol] | 191.0044 | 240 | 209.0555 |
| Q9UJA2 | E21gh3 | chromosome 20 open reading fra [symbol] | 134.2553 | 316 | 209.227 |
| Q02246 | J21ef6 | contactin 2 (axonal) | 108.0043 | 68.8 | 209.3915 |
| P48553 | E06cd2 | transmembrane protein 1 | 148.0375 | 238 | 209.4168 |
| Q99639 | K03cd2 | suppressor of Ty 5 homolog (S. [symbol] | 225.1962 | 296 | 209.795 |
| Q9NQX5 | N02gh4 | neural proliferation; differentiatio [symbol] | 188.0695 | 206 | 209.8245 |
| Q99799 | O08cd6 | nuclear RNA export factor 1 | 205.4132 | 175 | 209.9237 |
| Q9BZV1 | J05gh7 | UBX domain-containing 1 | 127.7069 | 236 | 209.9733 |
| Q03113 | M19ab5 | guanine nucleotide binding prote [symbol] | 140.2412 | 112 | 209.9975 |
| P01130 | B21ab6 | low density lipoprotein receptor ( [symbol] | 301.1179 | 394 | 210.369 |
| Q9UHN6 | L11cd8 | transmembrane protein 2 | 182.3012 | 130 | 210.5088 |
| Q99738 | A03cd3 | pinin; desmosome associated pr [symbol] | 200.7164 | 204 | 210.6944 |
| Q9Y576 | N03ef2 | ankyrin repeat and SOCS box-cc | 121.1139 | 76.2 | 210.8606 |
| P02304 | B03gh6 | H4 histone; family 2 | 81.15302 | 191 | 210.8651 |
| P01118 | I08ab7 | v-Ki-ras2 Kirsten rat sarcoma 2 v [symbol] | 209.4177 | 239 | 211.0234 |
| P15924 | K13ef7 | desmoplakin (DPI; DPII) | 249.9083 | 263 | 211.0334 |
| O43597 | A19cd6 | sprouty homolog 2 (Drosophila) | 119.5051 | 155 | 211.0573 |
| P09622 | I06ab3 | dihydrolipoamide dehydrogenas [symbol] | 346.3029 | 232 | 211.1679 |
| Q08257 | G16ef7 | crystallin; zeta (quinone reducta [symbol] | 514.6998 | 301 | 211.6448 |
| Q9UHY8 | J09cd7 | fasciculation and elongation pro [symbol] | 86.8735 | 95.1 | 211.8371 |
| P10646 | P23cd2 | tissue factor pathway inhibitor (li [symbol] | 596.8869 | 458 | 212.0146 |
| Q92685 | L08cd5 | Not56 (D. melanogaster)-like pro [symbol] | 195.2017 | 217 | 212.2397 |
| P34949 | I13ab7 | mannose phosphate isomerase | 102.2281 | 135 | 212.3424 |
| Q9NPE9 | M21ef8 | HSPC055 protein | 91.56792 | 157 | 212.3544 |
| Q99853 | J24ef7 | forkhead box B1 | 141.4272 | 105 | 212.4773 |
| Q9HD41 | C24gh4 | CHMP1.5 protein | 280.2988 | 230 | 212.8608 |
| Q16625 | A10ab8 | occludin | 859.5688 | 338 | 212.9222 |
| P45973 | L18ef6 | chromobox homolog 5 (HP1 alph [symbol] | 164.9514 | 124 | 213.1315 |
| Q9NVI9 | O12gh3 | hypothetical protein FLJ10707 | 136.0971 | 136 | 213.1484 |
| P37173 | O09ef7 | transforming growth factor; beta [symbol] | 112.4415 | 208 | 213.3211 |
| Q9UKF7 | O21cd8 | retinal degeneration B beta | 333.6061 | 287 | 213.5389 |
| P16870 | J23ef7 | carboxypeptidase E | 81.85341 | 137 | 213.5973 |
| P00374 | D16ab3 | dihydrofolate reductase | 148.8142 | 59.6 | 213.6217 |
| P19957 | M04ab8 | protease inhibitor 3; skin-dervied | 202.9357 | 582 | 213.9068 |
| O75962 | J24cd2 | triple functional domain (PTPRF | 233.7849 | 197 | 213.9409 |
| P16050 | C24ab2 | arachidonate 15-lipoxygenase | 288.5504 | 87.3 | 213.9824 |
| Q9Y3D2 | N14cd7 | pilin-like transcription factor | 200.9397 | 224 | 214.0069 |
| O00505 | J16ab6 | karyopherin alpha 3 (importin alp [symbol] | 358.8945 | 313 | 214.0819 |
| Q9NZD8 | N15ef1 | acid cluster protein 33 | 222.816 | 223 | 214.2958 |
| P46937 | G10cd6 | Yes-associated protein 1; 65 kD | 219.0427 | 268 | 214.3846 |
| P53384 | O05ab7 | nucleotide binding protein 1 (Min | 272.4995 | 223 | 214.5051 |
| Q9UI28 | K10ef4 | AD-003 protein | 120.4834 | 147 | 214.7067 |
| Q14651 | O20ab8 | plastin 1 (I isoform) | 517.7448 | 303 | 214.7993 |
| P10914 | K01ef6 | interferon regulatory factor 1 | 310.699 | 395 | 214.9514 |
| P21281 | A03ab3 | ATPase; H+ transporting; lysoso [symbol] | 299.07 | 638 | 215.0012 |
| P53701 | G19ab6 | holocytochrome c synthase (cyto [symbol] | 251.8825 | 415 | 215.1177 |
| Q9Y2B1 | C18ef3 | transmembrane protein 5 | 103.4737 | 123 | 215.2492 |
| Q14166 | D23ef8 | KIAA0153 protein | 332.5834 | 276 | 215.4465 |
| Q15738 | B09ef4 | NAD(P) dependent steroid dehy [symbol] | 253.061 | 171 | 215.5558 |
| Q16586 | I06cd1 | sarcoglycan; alpha (50 kD dystro [symbol] | 358.221 | 129 | 215.8058 |
| Q9Y6J8 | L05ef2 | map kinase phosphatase-like pr [symbol] | 275.0054 | 326 | 215.9987 |
| Q9H5F2 | P17gh5 | hypothetical protein FLJ23499 | 204.1242 | 332 | 216.1178 |
| Q05655 | E02cd1 | protein kinase C; delta | 419.7056 | 297 | 216.1184 |
| Q9NXU7 | M03gh3 | hypothetical protein FLJ20048 | 240.4802 | 235 | 216.1338 |
| O15498 | P21cd6 | SNARE protein Ykt6 | 188.9561 | 242 | 216.7243 |
| Q92664 | F07ab4 | general transcription factor IIIA | 318.1862 | 193 | 216.7575 |
| Q9Y223 | N03cd5 | UDP-N-acetylglucosamine-2-epi [symbol] | 207.311 | 295 | 217.1156 |
| O95816 | E21ab3 | BCL2-associated athanogene 2 | 84.7224 | 94.2 | 217.4827 |
| Q9H0K5 | A21gh8 | hypothetical protein DKFZp434H | 193.1934 | 324 | 217.6924 |
| Q9BTJ1 | C03ef2 | ribosomal protein S27-like | 132.1497 | 411 | 218.2702 |
| Q9NRR5 | J23gh4 | chromosome 1 open reading fra [symbol] | 138.2192 | 201 | 218.4149 |
| Q9Y5L3 | A13ab4 | ectonucleoside triphosphate dip [symbol] | 235.2745 | 316 | 218.4547 |
| O75351 | K06cd5 | suppressor of K+ transport defec [symbol] | 274.5936 | 311 | 218.6389 |
| O75443 | F07cd2 | tectorin alpha | 183.4591 | 150 | 218.7282 |
| Q9H7X4 | K04gh7 | KIAA1085 protein | 268.2576 | 372 | 218.7527 |
| O00255 | K22ab6 | multiple endocrine neoplasia I | 134.7209 | 117 | 218.9335 |
| Q9BV94 | D05gh3 | chromosome 20 open reading fr [symbol] | 319.259 | 316 | 219.2324 |
| O14813 | D10ab2 | aristaless homeobox (Drosophila | 226.3067 | 320 | 219.2567 |
| P35269 | D15ab4 | general transcription factor IIF; p [symbol] | 243.8395 | 213 | 219.8975 |
| Q12891 | E08ef5 | hyaluronoglucosaminidase 2 | 331.3833 | 311 | 220.4462 |
| O15162 | L08gh1 | phospholipid scramblase 1 | 440.6448 | 597 | 220.6688 |
| Q14469 | O08ab7 | hairy homolog (Drosophila) | 151.2148 | 251 | 221.0242 |
| P06241 | P13ab4 | FYN oncogene related to SRC; [symbol] | 106.4214 | 82.5 | 221.2951 |
| Q9BVG9 | P17gh7 | phosphatidylserine synthase 2 | 225.5812 | 200 | 221.5588 |
| Q9P0P9 | K12ef2 | hypothetical protein LOC51249 | 290.4444 | 434 | 222.6323 |
| Q9UMF9 | L14cd3 | eukaryotic translation initiation fa [symbol] | 207.6515 | 242 | 222.6344 |
| Q03527 | L07ab8 | proteasome (prosome; macropai [symbol] | 441.6371 | 284 | 222.6794 |
| P31153 | J15ab7 | methionine adenosyltransferase | 245.2111 | 134 | 222.7802 |
| Q04741 | D21ef1 | empty spiracles homolog 1 (Dros [symbol] | 240.3769 | 189 | 222.791 |
| Q13153 | L22ef5 | p21/Cdc42/Rac1-activated kinas [symbol] | 243.198 | 277 | 222.8583 |
| Q9Y388 | H15ef2 | CGI-79 protein | 100.7214 | 181 | 223.2467 |
| Q9HC14 | K17gh5 | mitochondrial ribosomal protein [symbol] | 158.7783 | 200 | 223.2594 |
| P30281 | O08ef5 | cyclin D3 | 107.4859 | 167 | 223.2751 |
| Q9UFK2 | P05ef8 | DKFZP564K1964 protein | 133.0476 | 210 | 223.4229 |
| Q13011 | O23ab4 | enoyl Coenzyme A hydratase 1; [symbol] | 337.9818 | 273 | 223.7187 |
| P09601 | E20ef7 | heme oxygenase (decycling) 1 | 186.4519 | 425 | 223.7447 |
| Q9BVZ8 | P23gh8 | hypothetical protein FLJ14936 | 126.689 | 127 | 224.0692 |
| Q13889 | A11ab5 | general transcription factor IIH; p [symbol] | 112.1935 | 202 | 224.1653 |
| P49703 | P23ab2 | ADP-ribosylation factor 4-like | 125.6732 | 91.2 | 224.1896 |
| Q9GZU7 | G11gh4 | nuclear LIM interactor-interactin [symbol] | 305.5546 | 150 | 224.2596 |
| P10253 | M22ab3 | glucosidase; alpha; acid (Pompe | 187.3998 | 156 | 224.2782 |
| Q9Y3V5 | B18ef8 | hypothetical protein DKFZP586F | 303.8477 | 426 | 224.4103 |
| O14773 | P17ef7 | ceroid-lipofuscinosis; neuronal 2 [symbol] | 169.6468 | 214 | 224.4535 |
| P22061 | G13cd1 | protein-L-isoaspartate (D-aspart [symbol] | 118.1783 | 163 | 224.5569 |
| P14868 | K21ab4 | aspartyl-tRNA synthetase | 117.1946 | 78.1 | 225.0996 |
| Q14938 | D16ef6 | nuclear factor I/X (CCAAT-bindin [symbol] | 94.58773 | 84.9 | 225.3458 |
| O75968 | P21ef3 | pannexin 1 | 215.9189 | 216 | 225.5368 |
| Q9H779 | C15gh6 | hypothetical protein MGC2821 | 138.4597 | 313 | 225.8086 |
| Q9BX72 | C02gh8 | IFP38 | 168.9682 | 172 | 225.8249 |
| O43563 | G24cd1 | solute carrier family 22 (organic [symbol] | 133.7352 | 149 | 225.8428 |
| O15142 | D17ab2 | ARP2 actin-related protein 2 ho [symbol] | 250.3086 | 215 | 226.0552 |
| Q9UJ41 | K17ef4 | putative Rab5 GDP/GTP exchan [symbol] | 121.9096 | 165 | 226.5205 |
| P05231 | F21ef7 | interleukin 6 (interferon; beta 2) | 240.2349 | 673 | 226.529 |
| Q9Y2H0 | G24ef8 | KIAA0964 protein | 279.6086 | 310 | 226.545 |
| Q9UJT9 | E15cd8 | F-box and leucine-rich repeat pr [symbol] | 287.9128 | 294 | 226.762 |
| Q9Y306 | O23ef2 | pelota homolog (Drosophila) | 146.8037 | 190 | 226.8083 |
| Q9Y6A6 | N04ef3 | transcription factor (p38 interacti [symbol] | 174.5425 | 171 | 226.9539 |
| O75300 | K18cd2 | ribosome binding protein 1 homo | 98.67954 | 73.7 | 227.0651 |
| P51798 | L03ef1 | chloride channel 7 | 278.4282 | 282 | 228.319 |
| Q14839 | C21ab4 | chromodomain helicase DNA bin [symbol] | 104.8847 | 161 | 228.322 |
| O60299 | O18gh1 | KIAA0552 gene product | 132.5981 | 337 | 228.426 |
| Q9Y6F1 | H03ab2 | ADP-ribosyltransferase (NAD+; [symbol] | 248.9844 | 399 | 228.5355 |
| O15212 | E04ef3 | HLA class II region expressed g [symbol] | 250.113 | 292 | 228.5629 |
| Q9Y2W2 | J14ef2 | WW domain binding protein 11 | 335.0512 | 304 | 228.6314 |
| Q9UKN5 | O03cd8 | PR domain containing 4 | 252.6208 | 80.6 | 228.639 |
| Q00978 | C24cd6 | interferon-stimulated transcriptio [symbol] | 303.7171 | 404 | 228.7768 |
| Q9UHY0 | L08ef1 | HP1-BP74 | 295.9512 | 267 | 228.8435 |
| Q9Y248 | L13ef2 | HSPC037 protein | 92.39515 | 70.3 | 229.0541 |
| O96019 | E21ab2 | BAF53 | 228.9673 | 345 | 229.5696 |
| P10276 | K19ab8 | retinoic acid receptor; alpha | 250.0312 | 122 | 229.7466 |
| Q9BUC7 | P22gh8 | hypothetical protein MGC4172 | 136.4413 | 398 | 229.8471 |
| Q9HAC1 | D01gh5 | praja 1 | 143.4188 | 97.5 | 230.0154 |
| Q93078 | M18gh6 | H2B histone family; member H | 179.9656 | 203 | 230.2553 |
| P08572 | N22ef6 | collagen; type IV; alpha 2 | 70.14126 | 87.3 | 230.3868 |
| P15056 | G10ef5 | v-raf murine sarcoma viral oncog [symbol] | 194.1803 | 174 | 231.0687 |
| Q15543 | J15cd2 | TAF13 RNA polymerase II; TAT [symbol] | 222.3129 | 262 | 231.2695 |
| O14901 | J05cd3 | TGFB inducible early growth res [symbol] | 277.9069 | 277 | 231.5821 |
| | N10gh8 | diphosphate dimethylallyl diphos [symbol] | 313.8915 | 257 | 231.841 |
| P51965 | O13cd3 | ubiquitin-conjugating enzyme E2 | 254.2693 | 673 | 232.1178 |
| Q9Y3R2 | G04cd8 | transportin-SR | 338.45 | 219 | 232.2301 |
| O94829 | G10gh1 | importin 13 | 160.8646 | 247 | 232.3109 |
| Q92859 | M13ab8 | neogenin homolog 1 (chicken) | 153.0455 | 249 | 232.6926 |
| Q12846 | O02cd2 | syntaxin 4A (placental) | 323.5547 | 93.2 | 232.9973 |
| P01286 | G15ef1 | growth hormone releasing hormo [symbol] | 237.6384 | 263 | 233.028 |
| Q9UH92 | L01cd8 | transcription factor-like 4 | 172.7135 | 195 | 233.1639 |
| O75323 | P09ab5 | glioblastoma amplified sequence | 178.3893 | 168 | 233.5843 |
| Q9UQ33 | P07ef2 | microtubule-associated protein; [symbol] | 101.7866 | 134 | 234.2195 |
| Q9Y3A7 | A17ef2 | CGI-101 protein | 402.1639 | 210 | 234.3057 |
| Q9Y2Z0 | A17cd7 | suppressor of G2 allele of SKP1; | 184.6693 | 256 | 234.3223 |
| P56211 | F16ab2 | cyclic AMP phosphoprotein; 19 k | 276.9734 | 386 | 234.3401 |
| P21127 | O13ef1 | cell division cycle 2-like 2 | 159.1108 | 304 | 234.454 |
| Q07866 | B23ab7 | kinesin 2 (60-70 kD) | 254.5932 | 408 | 234.5156 |
| Q9BTI3 | H11ef8 | KIAA1068 protein | 228.7025 | 230 | 234.5211 |
| Q9H2J4 | E11gh6 | hypothetical protein MGC3062 | 238.7573 | 256 | 235.0353 |
| Q9NXE5 | E10gh2 | hypothetical protein FLJ20296 | 193.8379 | 399 | 235.1468 |
| Q92572 | E17ab4 | adaptor-related protein complex: [symbol] | 86.23952 | 209 | 235.2813 |
| Q9NXT6 | K05gh2 | FtsJ homolog 3 (E. coli) | 254.7485 | 277 | 235.4026 |
| O15321 | F05cd6 | transmembrane 9 superfamily m [symbol] | 295.1129 | 227 | 235.695 |
| O15383 | F07cd6 | HIV-1 Tat interactive protein 2; 3 [symbol] | 402.8554 | 444 | 235.7849 |
| O60403 | B06cd8 | olfactory receptor; family 10; sub [symbol] | 237.7465 | 262 | 235.906 |
| P49354 | K24ab4 | famesyltransferase; CAAX box; [symbol] | 173.9344 | 193 | 235.9155 |
| P48039 | B07ef6 | melatonin receptor 1A | 305.0267 | 208 | 236.1249 |
| O43562 | C08ab8 | solute carrier family 22 (organic [symbol] | 115.4101 | 125 | 236.1587 |
| Q9HC89 | N24ef8 | calpain 10 | 212.0136 | 242 | 236.247 |
| P00790 | M13ef1 | pepsinogen 5; group I (pepsinog [symbol] | 256.009 | 266 | 236.6918 |
| Q9NVP1 | G01cd6 | DEAD/H (Asp-Glu-Ala-Asp/His) [symbol] | 78.40555 | 89 | 236.7778 |
| Q9UBN1 | F23ef3 | calcium channel; voltage-depend [symbol] | 269.9719 | 360 | 237.0557 |
| P35659 | M22cd3 | DEK oncogene (DNA binding) | 80.02159 | 77.8 | 237.1934 |
| P54252 | N09ef6 | Machado-Joseph disease (spino [symbol] | 156.7549 | 274 | 237.2775 |
| Q14152 | L12cd3 | eukaryotic translation initiation fa [symbol] | 364.5131 | 268 | 237.302 |
| Q9Y314 | E16ef2 | eNOS interacting protein | 123.3313 | 219 | 237.3058 |
| Q9NRC7 | E23ef3 | sirtuin silent mating type informa [symbol] | 136.0486 | 328 | 237.5194 |
| P02533 | N02ef6 | keratin 14 (epidermolysis bullosa [symbol] | 203.4102 | 190 | 237.6951 |
| O60907 | J19cd2 | transducin (beta)-like 1 X-linked | 175.9553 | 171 | 237.7396 |
| P43155 | B16ab3 | carnitine acetyltransferase | 288.355 | 190 | 237.8908 |
| Q15077 | H08ab8 | pyrimidinergic receptor P2Y; G-p [symbol] | 389.9746 | 248 | 238.1475 |
| Q12840 | I06ab7 | kinesin family member 5A | 238.3605 | 282 | 238.2407 |
| P26599 | A05cd3 | polypyrimidine tract binding prot [symbol] | 160.4912 | 345 | 238.4 |
| Q9Y316 | M11ef2 | C21orf19-like protein | 450.6398 | 327 | 238.5981 |
| P24394 | O18ab6 | interleukin 4 receptor | 309.5199 | 330 | 238.6201 |
| P32418 | J22gh1 | solute carrier family 8 (sodium/c [symbol] | 171.4835 | 134 | 238.7225 |
| P42694 | N01ef3 | helicase with zinc finger domain | 165.7017 | 146 | 238.7561 |
| O60755 | L09cd3 | galanin receptor 3 | 162.6185 | 268 | 238.7669 |
| Q16204 | A18cd4 | DNA segment; single copy; prob [symbol] | 192.0586 | 225 | 238.8166 |
| Q12980 | N15cd7 | Conserved gene telomeric to alp [symbol] | 183.7714 | 174 | 238.9231 |
| P08559 | N16ab7 | pyruvate dehydrogenase (lipoam [symbol] | 375.1479 | 206 | 239.4879 |
| Q9Y2U9 | K02ef3 | host cell factor homolog | 127.5142 | 154 | 239.623 |
| Q9Y2T2 | P17cd7 | adaptor-related protein complex: [symbol] | 336.9686 | 395 | 239.6299 |
| Q13105 | K10cd3 | zinc finger protein 151 (pHZ-67) | 177.9188 | 243 | 240.018 |
| Q9H1J1 | J12gh5 | similar to yeast Upf3; variant A | 154.12 | 183 | 241.0316 |
| Q08752 | E01cd1 | peptidylprolyl isomerase D (cycl [symbol] | 446.371 | 203 | 241.0389 |
| P38159 | F14ab7 | RNA binding motif protein; X ch [symbol] | 342.4693 | 176 | 241.0429 |
| Q9Y337 | A12cd8 | kallikrein 5 | 215.5515 | 312 | 241.2144 |
| P25445 | A02ef6 | tumor necrosis factor receptor su [symbol] | 102.3524 | 215 | 241.2257 |
| Q9BZH1 | P02gh5 | membrane-spanning 4-domains; | 350.8825 | 212 | 241.5154 |
| O43633 | O12ef4 | putative breast adenocarcinoma | 308.4601 | 232 | 241.573 |
| Q99536 | B03cd6 | vesicle amine transport protein 1 | 222.8504 | 400 | 241.6698 |
| P24557 | N11cd1 | thromboxane A synthase 1 (plat [symbol] | 326.4482 | 236 | 241.6841 |
| O75369 | G16ab4 | filamin B; beta (actin binding pro [symbol] | 331.6975 | 459 | 241.7672 |
| P50453 | H10ab8 | serine (or cysteine) proteinase in [symbol] | 439.8605 | 370 | 242.4005 |
| P35680 | O18cd1 | transcription factor 2; hepatic; LF | 235.5021 | 174 | 242.5943 |
| Q03924 | I01gh7 | zinc finger protein 117 (HPF9) | 257.8571 | 301 | 242.7729 |
| Q9NRQ5 | F17gh4 | FN5 protein | 361.924 | 325 | 242.8257 |
| Q99867 | G12gh7 | tubulin; beta polypeptide 4; mem [symbol] | 376.7361 | 247 | 243.6773 |
| Q9H682 | B19gh7 | A20-binding inhibitor of NF-kapp [symbol] | 205.6095 | 365 | 243.7949 |
| O00148 | N12cd5 | DEAD/H (Asp-Glu-Ala-Asp/His) [symbol] | 259.196 | 140 | 244.0023 |
| | F11cd8 | TCF3 (E2A) fusion partner (in ch [symbol] | 170.7519 | 295 | 244.0153 |
| Q9UG51 | N05ef3 | anaphase-promoting complex su [symbol] | 274.4079 | 331 | 244.0324 |
| O75175 | F12cd8 | CCR4-NOT transcription comple [symbol] | 230.1579 | 253 | 244.0384 |
| Q9GZQ8 | C05gh2 | microtubule-associated proteins | 261.5987 | 395 | 244.0725 |
| P19438 | A04ef6 | tumor necrosis factor receptor su [symbol] | 233.3188 | 197 | 244.3384 |
| Q9NRR8 | O04gh4 | small protein effector 1 of Cdc42 | 338.2133 | 329 | 244.5729 |
| O95162 | N10ef8 | peroxisomal short-chain alcohol [symbol] | 154.9486 | 108 | 244.9155 |
| O95279 | J22cd3 | potassium channel; subfamily K; | 274.3417 | 127 | 244.9972 |
| Q9NTW4 | N21ef8 | DKFZP564A2416 protein | 403.4188 | 219 | 245.1953 |
| Q9NWM9 | H21gh2 | hypothetical protein FLJ20730 | 121.538 | 207 | 245.69 |
| P40121 | P23ab5 | capping protein (actin filament); [symbol] | 452.2006 | 414 | 245.8846 |
| Q92466 | I04ab3 | damage-specific DNA binding p [symbol] | 99.17314 | 220 | 246.0478 |
| O43365 | K05gh7 | homeo box A3 | 132.7369 | 196 | 246.2071 |
| Q9NVJ7 | O02gh3 | DEAD/H (Asp-Glu-Ala-Asp/His) [symbol] | 336.1927 | 284 | 246.3831 |
| Q9NVY5 | H08ef7 | serologically defined colon canc [symbol] | 182.4596 | 178 | 246.7633 |
| Q9BY40 | G12gh8 | CDA11 protein | 141.0763 | 131 | 246.8975 |
| Q9NZE2 | O06ef2 | hypothetical protein LOC51321 | 76.41026 | 135 | 247.0025 |
| P29275 | K11ab2 | adenosine A2b receptor | 354.5115 | 191 | 247.0615 |
| Q9NZE0 | P22ef1 | WW domain-containing adapter [symbol] | 82.72809 | 71.1 | 247.6094 |
| Q03111 | N03ab7 | myeloid/lymphoid or mixed-linea [symbol] | 225.1377 | 162 | 247.8275 |
| Q08499 | G21ef6 | phosphodiesterase 4D; cAMP-sp [symbol] | 125.5177 | 92.9 | 248.1727 |
| Q15628 | A20ef6 | TNFRSF1A-associated via death | 214.1641 | 250 | 248.2613 |
| Q9Y657 | C07cd7 | spindlin | 217.9487 | 202 | 248.4185 |
| Q9UHQ1 | I09cd8 | nuclear prelamin A recognition f [symbol] | 312.9614 | 303 | 248.5926 |
| P17980 | H24ef6 | proteasome (prosome; macropai [symbol] | 331.1488 | 193 | 248.6286 |
| P45877 | I19ab8 | peptidylprolyl isomerase C (cycl [symbol] | 165.0716 | 169 | 248.9851 |
| O95340 | A23cd5 | 3′-phosphoadenosine 5′-phosph [symbol] | 110.9876 | 88.6 | 249.0622 |
| Q9H9Z7 | F18gh5 | hypothetical protein FLJ12448 | 158.5794 | 279 | 249.1478 |
| O15172 | E11cd4 | phosphoserine phosphatase-like | 321.9068 | 291 | 249.3629 |
| P01298 | F05ab8 | pancreatic polypeptide | 544.0034 | 194 | 249.376 |
| Q13395 | J17cd2 | TAR (HIV) RNA binding protein 1 | 260.7056 | 234 | 249.3777 |
| O15551 | G23ab4 | claudin 3 | 279.9519 | 250 | 249.4618 |
| P55060 | G20ef6 | CSE1 chromosome segregation | 322.1588 | 276 | 249.5903 |
| O43286 | E13ab3 | UDP-Gal: betaGlcNAc beta 1; 4- [symbol] | 316.581 | 468 | 249.8072 |
| P04424 | J10ab2 | argininosuccinate lyase | 594.9833 | 269 | 250.046 |
| Q9NWR5 | C10gh3 | hypothetical protein FLJ20657 | 171.9643 | 252 | 250.1961 |
| Q9BUN8 | M21gh6 | hypothetical protein MGC3067 | 165.6904 | 175 | 250.2847 |
| P49760 | G01ab4 | CDC-like kinase 2 | 69.23701 | 97.1 | 250.3266 |
| Q9UEG3 | E01gh7 | myotubularin related protein 3 | 367.623 | 236 | 250.553 |
| Q9P0Y6 | N07ef1 | chromosome 3 open reading frar [symbol] | 268.1158 | 256 | 250.6098 |
| Q92535 | M06ab8 | phosphatidylinositol glycan; clas [symbol] | 218.2502 | 172 | 250.9192 |
| Q00577 | A24gh7 | purine-rich element binding prot [symbol] | 839.6 | 273 | 251.0214 |
| O75717 | L21ab2 | AND-1 protein | 225.9974 | 250 | 251.1522 |
| P04278 | L09cd1 | sex hormone-binding globulin | 215.6067 | 196 | 251.1944 |
| P51124 | G19ab5 | granzyme M (lymphocyte met-as [symbol] | 108.9072 | 114 | 251.2498 |
| P15259 | P04ab7 | phosphoglycerate mutase 2 (mu [symbol] | 333.2442 | 363 | 251.6606 |
| Q12893 | O13cd7 | PL6 protein | 174.3077 | 280 | 252.0241 |
| P57738 | A11gh2 | T-cell leukemia transiocation aite [symbol] | 166.5683 | 232 | 252.2165 |
| P24863 | O22ef5 | cyclin C | 668.8634 | 523 | 252.3632 |
| Q9Y370 | H03ef2 | CGI-60 protein | 153.6349 | 157 | 252.5036 |
| Q9HD89 | E04gh4 | found in inflammatory zone 3 | 189.8748 | 182 | 252.7224 |
| O14807 | N04cd7 | muscle RAS oncogene homolog | 105.1243 | 181 | 252.8147 |
| Q13435 | I07cd7 | splicing factor 3b; subunit 2; 145 [symbol] | 270.3354 | 268 | 252.8473 |
| Q9UMY4 | F19cd8 | sorting nexin 12 | 178.8659 | 227 | 253.0136 |
| O43929 | C04ab8 | origin recognition complex; subu [symbol] | 267.5605 | 354 | 253.265 |
| Q9BZQ1 | H14gh7 | chromosome 1 open reading frar [symbol] | 219.6632 | 362 | 253.5183 |
| P55042 | K03ef5 | Ras-related associated with diab [symbol] | 300.5794 | 514 | 253.5279 |
| Q92870 | k18ef1 | amyloid beta (A4) precursor prot [symbol] | 76.07104 | 77.9 | 253.5714 |
| P33778 | C19gh7 | H2B histone family; member F | 169.4431 | 391 | 253.9554 |
| Q9ULZ3 | K22cd8 | apoptosis-associated speck-like | 255.9554 | 517 | 254.0856 |
| Q13485 | A20ef5 | MAD; mothers against decapent [symbol] | 163.1373 | 181 | 254.1546 |
| Q9H684 | C24gh5 | zinc finger protein 335 | 326.6826 | 261 | 254.3385 |
| Q9H7F8 | O06gh7 | hypothetical protein FLJ20958 | 190.1073 | 258 | 254.7196 |
| Q92508 | B17gh1 | KIAA0233 gene product | 241.1859 | 216 | 255.0491 |
| Q9UM47 | C15ab8 | Notch homolog 3 (Drosophila) | 64.97745 | 132 | 255.0839 |
| P18084 | E17ef7 | integrin; beta 5 | 259.2818 | 129 | 255.1335 |
| Q99462 | K22gh6 | ubiquitin-conjugating enzyme E2 | 207.6691 | 179 | 255.2632 |
| O75619 | J10ef7 | RNA helicase-related protein | 198.1059 | 335 | 255.6713 |
| Q92733 | K07cd1 | papillary renal cell carcinoma (tr [symbol] | 200.3207 | 260 | 256.1407 |
| Q15796 | H21ab7 | MAD; mothers against decapent [symbol] | 376.8422 | 446 | 256.2171 |
| P15291 | E07ab3 | UDP-Gal: betaGlcNAc beta 1; 4- [symbol] | 411.9325 | 403 | 256.9006 |
| Q99417 | I07cd8 | c-myc binding protein | 264.1386 | 256 | 256.9088 |
| P06732 | D09ab4 | creatine kinase; muscle | 202.527 | 127 | 257.2272 |
| P50570 | K13ab5 | dynamin 2 | 248.1389 | 228 | 257.2474 |
| Q9Y5A9 | F20ef2 | high-glucose-regulated protein 8 | 359.385 | 371 | 257.3917 |
| Q92841 | B17cd6 | DEAD/H (Asp-Glu-Ala-Asp/His) t [symbol] | 229.8916 | 270 | 257.9255 |
| P51808 | B22cd2 | t-complex-associated-testis-exp [symbol] | 182.2992 | 245 | 257.9487 |
| O95758 | H23cd5 | ROD1 regulator of differentiation | 411.9888 | 462 | 258.1758 |
| P32970 | I15ef5 | tumor necrosis factor (ligand) su [symbol] | 443.4158 | 456 | 258.4229 |
| O75689 | K21cd7 | centaurin; alpha 1 | 191.7263 | 327 | 258.6362 |
| P55072 | O06cd4 | valosin-containing protein | 466.7034 | 363 | 258.8411 |
| P47813 | A16ab4 | eukaryotic translation initiation fa [symbol] | 486.6493 | 337 | 259.0481 |
| Q15532 | J03cd2 | synovial sarcoma translocation; [symbol] | 190.8535 | 212 | 259.1488 |
| Q15813 | M19cd2 | tubulin-specific chaperone [symbol] | 151.2337 | 166 | 259.2747 |
| P14373 | B08cd2 | ret finger protein | 282.5757 | 270 | 259.3599 |
| O14980 | G02cd3 | exportin 1 (CRM1 homolog; yeas [symbol] | 239.4965 | 207 | 259.7187 |
| P07942 | P20ef6 | laminin; beta 1 | 71.03574 | 92.4 | 259.7579 |
| P53041 | C20cd1 | protein phosphatase 5; catalytic [symbol] | 272.5094 | 196 | 259.8136 |
| Q07820 | E12ef6 | myeloid cell leukemia sequence | 362.5007 | 243 | 260.0329 |
| P53816 | E04cd7 | HRAS-like suppressor 3 | 606.0957 | 558 | 260.1565 |
| Q9P1C8 | E09gh4 | hypothetical protein PRO2577 | 238.9909 | 205 | 260.3147 |
| P51966 | O21cd3 | ubiquitin-conjugating enzyme E2 | 148.3528 | 243 | 260.3185 |
| Q99739 | M03cd7 | plm-2 oncogene | 178.694 | 274 | 260.4482 |
| P35913 | N14ab7 | phosphodiesterase 6B; cGMP-s [symbol] | 199.3654 | 173 | 260.5708 |
| P52756 | J24cd5 | RNA binding motif protein 5 | 280.6537 | 230 | 261.0637 |
| Q9NXX5 | K17gh3 | hypothetical protein FLJ20004 | 130.163 | 197 | 261.2642 |
| Q14498 | O06cd5 | RNA-binding region (RNP1; RR [symbol] | 196.0463 | 216 | 261.5829 |
| P28069 | A03ab8 | POU domain; class 1; transcripti [symbol] | 261.1822 | 206 | 261.7763 |
| P51397 | E20ab5 | death-associated protein | 348.1009 | 312 | 261.9342 |
| Q9NUI8 | C24gh7 | putative methyltransferase | 157.3551 | 329 | 262.0287 |
| O00322 | M09cd7 | uroplakin 1A | 223.5286 | 295 | 262.0828 |
| P78368 | I09ab4 | casein kinase 1; gamma 2 | 211.1048 | 272 | 262.1137 |
| Q01081 | I22cd4 | U2(RNU2) small nuclear RNA au [symbol] | 138.1533 | 91.3 | 262.1811 |
| O43193 | J15ab5 | G protein-coupled receptor 38 | 69.57316 | 206 | 262.301 |
| Q9Y3B0 | M03ef2 | CGI-105 protein | 174.5026 | 177 | 262.5458 |
| Q9NV80 | H05gh3 | WD repeat domain 12 | 178.5139 | 279 | 262.6286 |
| Q9Y473 | O18cd4 | zinc finger protein 175 | 141.3794 | 275 | 263.1099 |
| Q9Y548 | L20gh2 | hypothetical protein DJ167A19.1 | 233.5442 | 240 | 263.4412 |
| Q13952 | N15ef6 | nuclear transcription factor Y; ga [symbol] | 241.7469 | 354 | 263.6055 |
| P18615 | K17cd3 | RD RNA-binding protein | 237.0199 | 242 | 263.7642 |
| P33241 | B23ef1 | lymphocyte-specific protein 1 | 94.80267 | 131 | 264.242 |
| O15116 | C01ef4 | Lsm1 protein | 344.8146 | 479 | 264.6979 |
| P50897 | A11ab8 | palmitoyl-protein thioesterase 1 ( [symbol] | 126.6311 | 142 | 264.8236 |
| | F20gh8 | MADP-1 protein | 134.6124 | 228 | 264.9564 |
| Q9Y3A5 | G12ef2 | CGI-97 protein | 469.4567 | 544 | 265.0744 |
| Q9BVT1 | G03gh6 | leukocyte receptor cluster (LRC) | 282.5457 | 328 | 265.5443 |
| Q9UM44 | E10cd7 | HERV-H LTR-associating 2 | 180.7238 | 119 | 265.5625 |
| Q9UGC7 | A05gh3 | similar to prokaryotic-type Class I | 289.9756 | 362 | 265.7778 |
| P49146 | G01ab8 | neuropeptide Y receptor Y2 | 222.0084 | 57.8 | 265.9266 |
| P54687 | I03ab3 | branched chain aminotransferas [symbol] | 103.4218 | 93.8 | 266.0547 |
| O60884 | O09cd6 | DnaJ (Hsp40) homolog; subfamil [symbol] | 330.3137 | 367 | 266.0819 |
| Q9NVC6 | J01cd7 | cofactor required for Sp1 transcri [symbol] | 183.0562 | 150 | 266.1556 |
| O43399 | G06cd2 | tumor protein D52-like 2 | 283.4636 | 348 | 266.2168 |
| O75880 | K22cd2 | SCO cytochrome oxidase deficie [symbol] | 156.7184 | 117 | 266.2419 |
| P49406 | F01gh1 | mitochondrial ribosomal protein [symbol] | 577.0339 | 353 | 266.3918 |
| Q9NYK5 | E15gh2 | mitochondrial ribosomal protein [symbol] | 473.2712 | 449 | 266.7642 |
| O15428 | P18cd2 | protein (peptidyl-prolyl cis/trans [symbol] | 185.1313 | 249 | 267.1449 |
| O14842 | K04ab5 | G protein-coupled receptor 40 | 222.6002 | 170 | 267.4241 |
| O15262 | I06cd6 | ring finger protein 3 | 243.845 | 401 | 267.4275 |
| Q9NP61 | J01ef3 | ADP-ribosylation factor GTPase [symbol] | 344.6989 | 175 | 267.443 |
| O15491 | P22cd5 | WD-repeat protein | 254.6166 | 355 | 267.6951 |
| O75390 | P01ab5 | citrate synthase | 656.561 | 312 | 268.0488 |
| P57088 | I12gh3 | hypothetical protein FLJ10525 | 377.5067 | 349 | 268.1833 |
| Q9NRY6 | K06gh4 | phospholipid scramblase 3 | 272.0774 | 334 | 268.2248 |
| O15403 | E05cd5 | solute carrier family 16 (monocar [symbol] | 384.2992 | 73.5 | 268.3423 |
| Q9BPZ5 | K09gh6 | hypothetical protein MGC3123 | 127.3745 | 286 | 268.7928 |
| P10301 | P01cd2 | related RAS viral (r-ras) oncoger | 314.4978 | 204 | 268.8367 |
| | A20gh7 | HLA-B associated transcript 4 | 168.0802 | 234 | 269.0656 |
| Q9P018 | A08ef8 | HSPC142 protein | 196.1899 | 346 | 269.5447 |
| Q9Y4H2 | L10cd3 | insulin receptor substrate 2 | 297.029 | 329 | 270.4962 |
| Q9UJX3 | F04ef2 | anaphase-promoting complex su [symbol] | 210.2906 | 204 | 270.642 |
| Q9H5X0 | O01gh6 | hypothetical protein MGC2941 | 333.5834 | 308 | 270.759 |
| Q92820 | H23cd4 | gamma-glutamyl hydrolase (conj [symbol] | 78.13783 | 101 | 271.925 |
| Q9UKK3 | K15ab2 | ADP-ribosyltransferase (NAD+; [symbol] | 337.297 | 450 | 271.9944 |
| Q9NWC8 | N13gh2 | hypothetical protein FLJ10120 | 315.7654 | 301 | 272.0013 |
| O14965 | I07cd2 | serine/threonine kinase 6 | 139.2727 | 101 | 272.158 |
| P50613 | B11ef5 | cyclin-dependent kinase 7 (MO1 [symbol] | 286.8535 | 423 | 272.2018 |
| Q9H1C4 | H08gh7 | unc-93 homolog B1 (C. elegans) | 348.8713 | 501 | 272.3385 |
| P21851 | E13ab4 | adaptor-related protein complex [symbol] | 338.7753 | 373 | 272.4617 |
| Q9UGJ0 | B14ef2 | protein kinase; AMP-activated; g [symbol] | 425.3015 | 543 | 272.5312 |
| P04035 | D17ab6 | 3-hydroxy-3-methylglutaryl-Coen [symbol] | 242.9132 | 169 | 273.2324 |
| Q9UKY7 | K03gh3 | hypothetical protein H41 | 643.0085 | 272 | 273.736 |
| O95478 | B18gh1 | hypothetical protein YR-29 | 248.2059 | 221 | 273.8047 |
| Q9Y3W9 | B16ef8 | sentrin/SUMO-specific protease [symbol] | 220.4064 | 333 | 274.1753 |
| Q9Y325 | A20ef2 | CGI-36 protein | 346.618 | 338 | 274.2434 |
| Q92854 | B09cd6 | sema domain; immunoglobulin d [symbol] | 342.6474 | 183 | 274.4146 |
| P16106 | O08gh6 | H3 histone family; member C | 105.4744 | 221 | 274.8424 |
| O43143 | M05ab4 | DEAD/H (Asp-Glu-Ala-Asp/His) [symbol] | 412.7015 | 349 | 274.9731 |
| Q13547 | L12ef6 | histone deacetylase 1 | 375.5725 | 372 | 275.2293 |
| Q9H6F9 | D11gh5 | hypothetical protein FLJ22313 | 306.5811 | 290 | 275.269 |
| O9NX18 | M08gh2 | hypothetical protein FLJ20487 | 311.71 | 394 | 275.6418 |
| O94764 | P03cd5 | RAN binding protein 9 | 256.3223 | 268 | 275.8144 |
| P82909 | N18gh8 | mitochondrial ribosomal protein [symbol] | 261.4618 | 343 | 275.8463 |
| Q9UI07 | D18ef8 | likely ortholog of mouse heal sh [symbol] | 199.8914 | 199 | 276.061 |
| P20337 | M01ef5 | RAB3B; member RAS oncogene [symbol] | 214.8304 | 126 | 276.148 |
| Q07960 | B22ab2 | Rho GTPase activating protein [symbol] | 219.98 | 218 | 276.184 |
| P49448 | P03cd7 | Glutamate dehydrogenase-2 | 344.3683 | 229 | 276.2751 |
| Q9NX48 | K08gh2 | hypothetical protein FLJ20442 | 493.7568 | 601 | 276.8343 |
| Q9NV56 | J03gh3 | chromosome 20 open reading fr [symbol] | 138.9543 | 104 | 277.4945 |
| O00154 | L21cd7 | brain acyl-CoA hydrolase | 173.5669 | 189 | 277.5737 |
| Q9UI70 | B04ef1 | nucleolar protein ANKT | 307.7657 | 349 | 277.6076 |
| P26045 | N17ab8 | protein tyrosine phosphatase; no | 412.292 | 315 | 277.6603 |
| Q9Y2A8 | C24ef3 | CD209 antigen-like | 72.21222 | 91.3 | 277.8103 |
| Q9BZ89 | F08gh7 | phospholipase A2; group XII | 175.384 | 216 | 277.9804 |
| P35052 | J24ab4 | glypican 1 | 214.4355 | 173 | 278.2085 |
| Q9H0S9 | K21gh7 | nuclear receptor binding factor-2 | 80.19669 | 155 | 278.4321 |
| Q15654 | I07ef7 | thyroid hormone receptor interac [symbol] | 264.295 | 238 | 278.8016 |
| P51948 | I09ab7 | menage a trois 1 (CAK assembly [symbol] | 239.1769 | 272 | 278.9368 |
| Q13133 | B20cd5 | nuclear receptor subfamily 1; gro [symbol] | 276.5554 | 260 | 279.1408 |
| Q9P0J9 | H22gh3 | goliath protein | 337.3229 | 215 | 279.5646 |
| Q15546 | G23cd8 | monocyte to macrophage differe [symbol] | 136.5038 | 94 | 279.6415 |
| P28325 | E09ab5 | cystatin D | 371.6792 | 309 | 279.7092 |
| O94985 | O04ef8 | calsyntenin 1 | 137.0471 | 205 | 279.921 |
| P48634 | M03cd4 | HLA-B associated transcript 2 | 266.8369 | 179 | 280.5497 |
| Q9BRL6 | O15gh7 | Splicing factor; arginine/serine- [symbol] | 220.3804 | 304 | 280.5881 |
| O94851 | E06gh1 | KIAA0750 gene product | 108.6672 | 172 | 280.9738 |
| Q9NW56 | E20gh3 | hypothetical protein FLJ10305 | 240.4538 | 314 | 281.002 |
| Q9H6X6 | P19gh5 | CasL interacting molecule | 183.3739 | 302 | 281.185 |
| P41217 | N13ab7 | antigen identified by monoclonal | 325.1642 | 166 | 281.3505 |
| P31512 | P04ab4 | flavin containing monooxygenas [symbol] | 192.4074 | 276 | 281.6555 |
| Q15425 | H05cd2 | SA hypertension-associated hor [symbol] | 186.2788 | 151 | 281.9205 |
| P40306 | L05ab8 | proteasome (prosome; macropai [symbol] | 408.3043 | 513 | 281.9429 |
| Q9NWZ8 | O02gh2 | hypothetical protein FLJ20514 | 287.569 | 146 | 281.9498 |
| P57073 | E22ef8 | SRY (sex determining region Y) [symbol] | 202.5886 | 268 | 281.9705 |
| Q9Y450 | H22cd6 | HBS1-like (S. cerevisiae) | 230.8266 | 312 | 282.4288 |
| P04062 | O08ab3 | glucosidase; beta; acid (includes | 295.5318 | 389 | 282.6453 |
| O75065 | E22gh1 | phosphodiesterase 4D interactin [symbol] | 168.0201 | 154 | 282.6456 |
| Q9ULX2 | F09ef3 | NIMA (never in mitosis gene a) [symbol] | 288.5578 | 208 | 283.1715 |
| Q99871 | C06ef4 | three prime repair exonuclease 2 | 205.6083 | 225 | 283.2504 |
| Q93065 | I15cd6 | PERB11 family member in MHC [symbol] | 295.7132 | 457 | 283.4269 |
| O43837 | B12ab7 | isocitrate dehydrogenase 3 (NA [symbol] | 348.9961 | 298 | 283.6422 |
| Q9H9L3 | L06gh7 | hypothetical protein FLJ12671 | 209.5906 | 279 | 283.6912 |
| Q13740 | A18ab2 | activated leucocyte cell adhesio [symbol] | 231.4555 | 253 | 283.7582 |
| Q12888 | L09cd2 | tumor protein p53 binding protei [symbol] | 132.9276 | 223 | 283.8572 |
| P78318 | J13ab6 | immunoglobulin (CD79A) binding | 423.8715 | 512 | 283.9511 |
| P48436 | K24cd1 | SRY (sex determining region Y)- [symbol] | 345.2087 | 197 | 284.0786 |
| P18065 | B19ef1 | insulin-like growth factor binding | 286.6516 | 283 | 284.2331 |
| Q9ULW6 | F22cd8 | nucleosome assembly protein 1- [symbol] | 223.0694 | 148 | 284.717 |
| O95154 | N03cd7 | aldo-keto reductase family 7; me [symbol] | 299.9079 | 298 | 284.8919 |
| O95169 | P14ab8 | NADH dehydrogenase (ubiquino [symbol] | 374.2624 | 199 | 285.0096 |
| Q99982 | C24ef4 | XIAP associated factor-1 | 63.10609 | 116 | 285.0252 |
| Q99470 | F14cd2 | stromal cell-derived factor 2 | 230.3684 | 316 | 285.1394 |
| P16104 | N19ab5 | H2A histone family; member X | 234.8351 | 195 | 285.2056 |
| Q9UG25 | B04ef8 | SOCS box-containing WD protei [symbol] | 213.293 | 398 | 285.5392 |
| Q13158 | C21cd4 | Fas (TNFRSF6)-associated via d [symbol] | 263.0157 | 281 | 285.9553 |
| Q9NW32 | D10gh2 | hypothetical protein FLJ10346 | 235.0532 | 386 | 286.3524 |
| O95153 | M05cd5 | peripheral benzodiazepine recep [symbol] | 155.1824 | 231 | 287.1271 |
| Q9BQ73 | H06gh7 | hypothetical protein FKSG28 | 212.0736 | 297 | 287.201 |
| Q15426 | C02ef7 | protein tyrosine phosphatase; re [symbol] | 296.9619 | 441 | 287.2583 |
| Q92858 | L12ab2 | atonal homolog 1 (Drosophila) | 611.3779 | 278 | 287.2641 |
| Q16849 | P15ab8 | protein tyrosine phosphatase; re [symbol] | 145.9896 | 55.6 | 287.3262 |
| P11279 | D07ab7 | lysosomal-associated membrane | 369.9925 | 310 | 287.3935 |
| Q96II1 | I16gh7 | hypothetical protein MGC14961 | 103.7251 | 80.7 | 287.4071 |
| P78504 | O20ab2 | jagged 1 (Alagille syndrome) | 255.0945 | 347 | 287.4633 |
| O75937 | G06ef3 | DnaJ (Hsp40) homolog; subfami [symbol] | 294.9992 | 264 | 287.503 |
| O60575 | O16ef4 | serine protease inhibitor; Kazal [symbol] | 142.2326 | 152 | 287.9 |
| Q9NYP7 | K05gh5 | homolog of yeast long chain poly [symbol] | 299.2029 | 187 | 288.1627 |
| O95436 | H01cd6 | solute carrier family 34 (sodium [symbol] | 238.6021 | 425 | 288.3247 |
| O95324 | B15ab2 | ATP-binding cassette; sub-family | 267.704 | 275 | 288.4558 |
| Q9UQF6 | B08ef3 | ribosomal protein L36 | 519.2003 | 425 | 288.5855 |
| P13010 | K12ef6 | X-ray repair complementing defe [symbol] | 174.6996 | 256 | 288.9405 |
| Q9UJZ1 | P03cd8 | stomatin (EPB72)-like 2 | 490.6115 | 272 | 289.2003 |
| P50281 | I18ab7 | matrix metalloproteinase 14 (mer [symbol] | 126.7204 | 196 | 289.8803 |
| P15927 | B08cd1 | replication protein A2 (32 kD) | 100.0172 | 194 | 290.3306 |
| O75496 | B02ef1 | geminin | 466.4376 | 454 | 290.7216 |
| Q9P1E3 | C23gh4 | presenilins associated rhomboid- [symbol] | 232.5496 | 263 | 291.2445 |
| P40189 | B14ab6 | interleukin 6 signal transducer (g [symbol] | 330.8834 | 315 | 291.3287 |
| Q9Y259 | H03ab4 | choline kinase-like | 328.1801 | 375 | 291.3288 |
| O00633 | C12ef5 | phosphatase and tensin homolog | 199.7323 | 189 | 291.6118 |
| Q13432 | H13cd5 | unc-119 homolog (C. elegans) | 127.1818 | 268 | 291.793 |
| Q9Y677 | E03ef2 | COP9 constitutive photomorpho [symbol] | 186.9367 | 204 | 291.9539 |
| Q9BW24 | D03ef8 | KIAA0676 protein | 170.3265 | 240 | 292.9836 |
| Q9NSD9 | B12cd5 | phenylalanyl-tRNA synthetase b [symbol] | 226.6641 | 219 | 293.111 |
| Q9NWQ0 | C16gh3 | hypothetical protein FLJ20695 | 497.4612 | 656 | 293.1504 |
| Q9NWT6 | C02gh3 | hypoxia-inducible factor 1; alpha | 521.7793 | 276 | 293.7543 |
| Q9UPN3 | J12ef7 | microtubule-actin crosslinking fa [symbol] | 578.3578 | 563 | 294.1533 |
| Q96HW0 | A06gh7 | zinc finger protein 38 (KOX 25) | 126.7599 | 355 | 294.3016 |
| Q14257 | F18ab8 | reticulocalbin 2; EF-hand calciu [symbol] | 237.969 | 217 | 295.1469 |
| Q9NRJ5 | J05gh4 | poly(A) polymerase beta (testis s [symbol] | 379.2857 | 276 | 295.7341 |
| P23280 | P02ab3 | carbonic anhydrase VI | 137.6155 | 102 | 296.1358 |
| Q14677 | I02gh1 | KIAA0171 gene product | 330.738 | 204 | 296.1674 |
| Q9Y394 | H17ef2 | CGI-86 protein | 471.0368 | 358 | 296.7589 |
| Q9UKX5 | L18cd7 | integrin; alpha 11 | 99.18133 | 126 | 297.0397 |
| Q92539 | G02gh1 | lipin 2 | 260.7493 | 235 | 297.1151 |
| Q9NW01 | G10gh3 | hypothetical protein FLJ10402 | 165.6121 | 135 | 297.1276 |
| Q9NUW5 | N21gh3 | hypothetical protein FLJ11099 | 228.3252 | 247 | 297.5163 |
| Q9BYC4 | C13gh8 | mitochondrial ribosomal protein [symbol] | 478.9122 | 408 | 297.754 |
| P02545 | F01ab7 | lamin A/C | 707.7874 | 294 | 297.9921 |
| O60585 | A17cd6 | serine/arginine repetitive matrix [symbol] | 318.399 | 331 | 298.2057 |
| P23458 | L12ef5 | Janus kinase 1 (a protein tyrosin | 385.7348 | 441 | 298.2124 |
| Q9BY13 | O06gh8 | hook3 protein | 435.0319 | 471 | 298.2969 |
| Q9UBC2 | C05gh5 | epidermal growth factor receptor | 171.9695 | 271 | 298.6179 |
| O76022 | A14cd7 | E1B-55 kDa-associated protein 5 | 256.9682 | 243 | 298.7615 |
| P33993 | K04ef6 | MCM7 minichromosome mainten [symbol] | 239.0601 | 165 | 298.875 |
| Q92963 | F02cd2 | Ric-like: expressed in many tissu [symbol] | 447.2008 | 499 | 299.9377 |
| Q9HAF1 | P09gh5 | hypothetical protein FLJ11730 | 270.4702 | 414 | 300.0397 |
| Q9NZ36 | P02gh4 | uncharacterized hypothalamus p [symbol] | 505.7323 | 333 | 300.1219 |
| Q9BQQ3 | C09gh8 | golgi phosphoprotein 5 | 208.3671 | 343 | 300.1696 |
| O95214 | P09ef3 | leptin receptor overlapping trans [symbol] | 292.4076 | 419 | 300.357 |
| Q9Y294 | A21ef8 | DKFZP547E2110 protein | 166.5906 | 243 | 301.1573 |
| O95810 | O01cd4 | serum deprivation response (phc [symbol] | 279.903 | 272 | 301.2133 |
| P30670 | L21ab4 | guanine nucleotide binding prote [symbol] | 470.8713 | 466 | 301.8899 |
| P51946 | O14ef5 | cyclin H | 180.4218 | 244 | 301.9337 |
| Q15290 | G18cd1 | retinoblastoma binding protein 6 | 298.4739 | 346 | 302.0906 |
| Q9NX11 | A03gh3 | hypothetical protein FLJ20498 | 165.3845 | 190 | 302.1175 |
| Q9P0S3 | A23ef2 | hypothetical protein LOC51240 | 187.2653 | 229 | 302.318 |
| P04554 | H11ab8 | protamine 2 | 393.1528 | 362 | 302.3989 |
| O15243 | O05ef4 | leptin receptor gene-related prot [symbol] | 329.3033 | 391 | 302.5417 |
| O43717 | G03cd7 | tumor differentially expressed 1 | 284.4848 | 364 | 302.9444 |
| O95876 | J10ef1 | hypothetical protein LOC51057 | 172.7128 | 226 | 302.9853 |
| Q9H8D5 | N17gh5 | tumor endothelial marker 6 | 247.105 | 190 | 303.4837 |
| Q9Y3B6 | D24ef1 | CGI-112 protein | 493.6755 | 288 | 303.7855 |
| Q9P0S7 | E13ef2 | hypothetical protein LOC51238 | 143.0704 | 139 | 303.896 |
| O60630 | D22ab4 | deltex homolog 1 (Drosophila) | 310.6617 | 221 | 303.9702 |
| O95285 | O07gh1 | macrophage erythroblast attache [symbol] | 332.2343 | 413 | 304.0714 |
| Q08945 | G17cd2 | structure specific recognition pro [symbol] | 335.05 | 229 | 304.6617 |
| O43583 | D08cd3 | density-regulated protein | 203.0088 | 253 | 304.7034 |
| O95149 | D04cd5 | RNA; U transporter 1 | 186.979 | 254 | 304.889 |
| Q9HAS8 | N10gh4 | elaC homolog 2 (E. coli) | 243.3076 | 331 | 304.8937 |
| Q9BQ39 | A23gh6 | nucleolar protein GU2 | 234.3353 | 160 | 305.2734 |
| O15304 | C06ef6 | CD27-binding (Siva) protein | 88.75727 | 153 | 305.4708 |
| Q15394 | I08gh1 | basic leucine-zipper protein BZA | 347.7168 | 384 | 305.6252 |
| Q9NZA3 | N09ef1 | CDA14 | 235.1955 | 312 | 305.7623 |
| P26022 | P23ab8 | pentaxin-related gene; rapidly in [symbol] | 97.61703 | 61.3 | 305.8598 |
| Q00536 | L24ef5 | PCTAIRE protein kinase 1 | 290.1914 | 359 | 306.0473 |
| Q9UMW5 | E19ef2 | cerebral cell adhesion molecule | 52.28353 | 234 | 306.6326 |
| O95353 | J17cd6 | Cdc42 effector protein 3 | 95.65314 | 208 | 306.7186 |
| O95808 | B03ef2 | arsenate resistance protein ARS [symbol] | 187.799 | 225 | 307.4311 |
| Q9BXJ8 | O17gh8 | transmembrane protein induced I | 411.8484 | 646 | 308.2116 |
| P36776 | A06cd5 | protease; serine; 15 | 390.4608 | 365 | 308.5755 |
| Q9BUR9 | M19gh6 | hypothetical protein MGC4614 | 250.3191 | 338 | 308.7431 |
| Q9Y3R1 | F23cd6 | Rab acceptor 1 (prenylated) | 158.9446 | 207 | 308.7948 |
| Q92997 | D03ef7 | dishevelled; dsh homolog 3 (Dro [symbol] | 240.0941 | 305 | 309.2314 |
| O14682 | N17cd3 | ectodermal-neural cortex (with B [symbol] | 326.0664 | 189 | 309.539 |
| Q9Y255 | I08cd8 | px19-like protein | 533.9688 | 455 | 309.7497 |
| Q16664 | O17ef4 | protein \A\** | 354.4762 | 286 | 310.2736 |
| P31943 | O04ab7 | heterogeneous nuclear ribonucle [symbol] | 690.0051 | 250 | 310.5901 |
| P49848 | J09cd2 | TAF6 RNA polymerase II; TATA | 151.1422 | 133 | 310.9274 |
| Q04984 | P19ab8 | heat shock 10 kD protein 1 (chap [symbol] | 338.1568 | 327 | 311.1011 |
| P20719 | N08ef4 | homeo box A5 | 271.4135 | 326 | 311.2742 |
| Q9H5Y0 | D21gh7 | hypothetical protein FLJ22795 | 325.1674 | 305 | 311.3753 |
| P19021 | G15ab8 | peptidylglycine alpha-amidating [symbol] | 219.5791 | 273 | 311.913 |
| Q92544 | B13gh1 | KIAA0255 gene product | 270.8313 | 307 | 312.0297 |
| Q12907 | G07cd7 | chromosome 5 open reading frar [symbol] | 313.0938 | 222 | 312.5092 |
| Q9BW91 | C03gh6 | nudix (nucleoside diphosphate li [symbol] | 346.395 | 314 | 312.5575 |
| P53365 | M23cd8 | partner of RAC1 (arfaptin 2) | 293.2566 | 349 | 312.9335 |
| P35869 | O17ab2 | aryl hydrocarbon receptor | 249.5731 | 417 | 313.1153 |
| Q13145 | I19cd8 | putative transmembrane protein | 490.8698 | 608 | 313.616 |
| O14747 | A18ef6 | death-associated protein 6 | 348.2382 | 450 | 313.7398 |
| Q9GZT5 | H03gh7 | wingless-type MMTV integration | 362.0025 | 327 | 313.9764 |
| P08174 | M19ef1 | decay accelerating factor for corr [symbol] | 705.5781 | 457 | 314.0939 |
| Q9H689 | K01gh4 | hypothetical protein RG083M05 [symbol] | 241.9641 | 291 | 314.1855 |
| Q9P0T7 | K05ef2 | hypothetical protein LOC51235 | 266.1575 | 324 | 314.3982 |
| Q9H2V7 | I12gh8 | spinster-like protein | 275.9977 | 394 | 314.4425 |
| Q9NWT0 | D23gh2 | hypothetical protein FLJ20625 | 420.628 | 488 | 314.6523 |
| Q9Y6X2 | E22cd6 | protein inhibitor of activated STA | 149.0425 | 237 | 315.009 |
| Q9P1P1 | F10ef8 | clone FLB1727 (LOC51215) | 419.9659 | 330 | 315.0092 |
| P23919 | H04cd7 | deoxythymidylate kinase (thymid [symbol] | 123.4582 | 84.1 | 315.562 |
| P37140 | D09ab8 | protein phosphatase 1; catalytic [symbol] | 355.0781 | 503 | 315.7751 |
| Q9NW54 | B22gh2 | homolog of rat nadrin | 432.3799 | 670 | 315.8314 |
| Q9UMS0 | C05ef4 | HIRA interacting protein 5 | 504.0153 | 382 | 315.8386 |
| P14207 | E23ef1 | folate receptor 2 (fetal) | 235.5291 | 154 | 316.0649 |
| Q9UHC7 | L05ef4 | makorin; ring finger protein; 1 | 243.752 | 243 | 316.3297 |
| O75844 | K23cd6 | zinc metalloproteinase (STE24 h | 338.221 | 174 | 316.3789 |
| | P18gh8 | LIM homeobox protein 4 | 87.11814 | 84.8 | 316.6454 |
| P16587 | P19ab2 | ADP-ribosylation factor 3 | 444.3724 | 348 | 317.1873 |
| Q9NVY2 | F18gh2 | hypothetical protein FLJ10439 | 420.4716 | 399 | 317.2936 |
| Q9Y3I1 | J09ef4 | F-box only protein 7 | 368.7457 | 422 | 317.6515 |
| Q9H320 | P21cd8 | variable charge; X chromosome | 147.0814 | 348 | 318.4769 |
| P49903 | J11ef4 | selenium donor protein | 329.031 | 446 | 318.9322 |
| O00506 | B05cd6 | serine/threonine kinase 25 (STE [symbol] | 273.696 | 275 | 319.3179 |
| O00273 | F14ab5 | DNA fragmentation factor; 45 kD | 328.993 | 401 | 319.634 |
| Q08170 | H11cd2 | splicing factor; arginine/seriene-ri [symbol] | 231.1994 | 193 | 319.7733 |
| Q9Y3B8 | N15ef8 | small fragment nuclease | 120.8094 | 282 | 320.0578 |
| Q9P0N0 | E11ef2 | mitochondrial ribosomal protein [symbol] | 377.6692 | 391 | 320.1382 |
| Q9BRI3 | D11gh8 | hypothetical protein MGC11303 [symbol] | 373.5695 | 391 | 320.4318 |
| Q9Y319 | D19ef2 | CGI-30 protein | 107.1253 | 123 | 320.4576 |
| Q16514 | M17gh1 | TAF12 RNA polymerase II; TATA | 116.3618 | 304 | 320.8611 |
| P13489 | D13ef7 | ribonuclease/angiogenin inhibito [symbol] | 224.1663 | 232 | 321.0895 |
| Q99987 | P08ef5 | vaccinia related kinase 2 | 205.9202 | 194 | 321.2364 |
| P56181 | H21ef1 | NADH dehydrogenase (ubiquino [symbol] | 435.0715 | 319 | 321.3696 |
| P51148 | N18ab8 | RAB5C; member RAS oncogene [symbol] | 329.7976 | 358 | 321.5906 |
| Q9HD34 | E12gh4 | CGI-203 protein | 257.866 | 249 | 322.345 |
| Q9HAN2 | F02gh6 | purnilio homolog 2 (Drosophila) | 291.9641 | 326 | 322.4096 |
| P08107 | K13ef5 | heat shock 70 kD protein 1A | 535.4871 | 382 | 322.6223 |
| O60499 | N18cd3 | syntaxin 10 | 189.7411 | 304 | 322.7738 |
| Q9HBL3 | M19gh4 | N-terminal kinase-like | 165.2938 | 224 | 323.2472 |
| Q9BSJB | D22gh6 | KIAA0747 protein | 549.1645 | 575 | 323.5208 |
| O60838 | G09gh7 | SAC2 suppressor of actin mutati [symbol] | 216.0571 | 254 | 324.4643 |
| Q98TM6 | M11gh8 | hypothetical protein MGC3195 | 371.9412 | 334 | 324.5103 |
| Q9H4L0 | K06gh5 | leucine zipper protein FKSG14 | 47.34403 | 70.6 | 324.6661 |
| P50402 | I14ab3 | emerin (Emery-Dreifuss muscula [symbol] | 321.3743 | 304 | 324.6751 |
| Q9NZP7 | G05ef3 | calcium binding protein Cab45 p [symbol] | 336.1107 | 239 | 324.6956 |
| P49116 | G22cd2 | nuclear receptor subfamily 2; gro [symbol] | 295.9342 | 224 | 325.1253 |
| P11234 | D14ab8 | v-ral sirrian leukemia viral oncog [symbol] | 565.699 | 444 | 325.5924 |
| P15408 | D21ab5 | FOS-like antigen 2 | 265.6989 | 437 | 325.8448 |
| Q9P1Q8 | N05cd8 | RAB3A interacting protein (rabin [symbol] | 357.8493 | 253 | 326.2784 |
| P39060 | P16ef6 | collagen; type XVIII; alpha 1 | 151.6584 | 212 | 326.8288 |
| Q9Y2N3 | N05gh1 | KIAA0618 gene product | 378.8717 | 252 | 326.8347 |
| P02749 | M22ab2 | apolipoprotein H (beta-2-glycopn [symbol] | 408.7454 | 226 | 327.1228 |
| Q9P189 | L20gh3 | hypothetical protein PRO1855 | 392.3328 | 358 | 327.1908 |
| O43822 | B19ab4 | chromosome 21 open reading fr [symbol] | 249.9987 | 297 | 327.8679 |
| Q9UNZ2 | N15ef2 | likely ortholog of rat p47 | 413.5167 | 519 | 327.9429 |
| Q9Y4G5 | B22cd8 | dynein; cytoplasmic; heavy poly [symbol] | 376.6624 | 426 | 327.9518 |
| Q13310 | C13cd4 | poly(A) binding protein; cytoplas [symbol] | 316.8746 | 338 | 328.1257 |
| P45974 | O14cd3 | ubiquitin specific protease 5 (iso [symbol] | 268.1073 | 316 | 328.8257 |
| P55317 | A04ab7 | hepatocyte nuclear factor 3; alph [symbol] | 273.7527 | 306 | 328.8562 |
| O43364 | H06cd8 | homeo box A2 | 511.053 | 468 | 329.1292 |
| P55345 | H15ab6 | HMT1 hnRNP methyltransferase | 364.8758 | 307 | 329.5023 |
| P48230 | O22cd2 | transmembrane 4 superfamily m [symbol] | 565.1635 | 417 | 330.825 |
| Q9BUY3 | C21gh5 | sorting nexin 6 | 166.5144 | 297 | 330.8949 |
| O00115 | B01ef6 | deoxyribonuclease II; lysosomal | 275.2503 | 372 | 331.0554 |
| O43318 | M11cd2 | mitogen-activated protein kinase | 186.7739 | 247 | 331.1308 |
| O95572 | B08gh1 | mitofusin 2 | 252.7846 | 277 | 331.4516 |
| P35080 | K12ab8 | profilin 2 | 111.4363 | 85.9 | 332.2299 |
| Q9NW29 | E24gh3 | hypothetical protein FLJ10350 | 219.5611 | 173 | 332.8499 |
| Q9NZE9 | O07ef2 | BRAF35/HDAC2 complex (80 kD [symbol] | 219.3916 | 348 | 332.8813 |
| Q9BYV2 C | E05gh8 | ring finger protein 30 | 500.2246 | 338 | 333.9675 |
| Q9UJX4 | F02ef2 | anaphase promoting complex sul [symbol] | 237.1218 | 269 | 334.5884 |
| O00302 | B19cd6 | calcium homeostasis endoplasm [symbol] | 327.1489 | 382 | 334.7371 |
| Q92886 | K23cd1 | neurogenin 1 | 290.2357 | 283 | 335.1054 |
| O14929 | P07cd3 | histone acetyltransferase 1 | 344.9153 | 130 | 335.4301 |
| Q9NVK7 | M18gh3 | chromosome 19 open reading fre [symbol] | 281.2043 | 287 | 335.4383 |
| Q9UBN0 | P09ef4 | proline rich 3 | 309.9093 | 344 | 335.4526 |
| O43611 | E14cd3 | cytoplasmic linker 2 | 180.9108 | 220 | 336.1459 |
| O94777 | G23cd4 | dolichyl-phosphate mannosyltran [symbol] | 294.1356 | 244 | 336.7183 |
| Q14669 | K05gh1 | thyroid hormone receptor interac [symbol] | 486.9829 | 397 | 337.0199 |
| Q9NUR0 | B20gh3 | hypothetical protein FLJ11196 | 63.09205 | 107 | 337.0769 |
| Q16822 | L08ab8 | phosphoenolpyruvate carboxykin [symbol] | 262.4601 | 156 | 337.1655 |
| Q9Y640 | A14cd8 | stromal cell derived factor recept [symbol] | 215.2348 | 220 | 337.2906 |
| P48595 | C01cd1 | serine (or cysteine) proteinase in [symbol] | 62.04001 | 410 | 337.3872 |
| P26639 | M15cd2 | threonyl-tRNA synthetase | 355.1309 | 422 | 337.8401 |
| P29597 | O03cd3 | tyrosine kinase 2 | 209.3427 | 258 | 338.1471 |
| O60671 | B02ab8 | RAD1 homolog (S. pombe) | 188.2716 | 104 | 339.1461 |
| Q9NW97 | P17gh2 | hypothetical protein FLJ10199 | 262.3124 | 536 | 339.3086 |
| Q9P027 | O23ef8 | HSPC133 protein | 616.0461 | 363 | 339.3102 |
| O95772 | E20gh8 | hypothetical protein MGC3251 | 195.9084 | 227 | 339.3844 |
| Q9NR28 | N13gh4 | second mitochondria-derived act [symbol] | 271.4457 | 543 | 339.8107 |
| P11413 | F15ab3 | glucose-6-phosphate dehydroge [symbol] | 258.0938 | 135 | 340.0217 |
| Q99567 | A04ab8 | nucleoporin 88 kD | 464.4826 | 320 | 340.5878 |
| Q9BZR9 | F22gh7 | ring finger protein 27 | 206.7918 | 398 | 340.7239 |
| O43632 | L22cd6 | gamma-tubulin complex protein 2 [symbol] | 328.0052 | 322 | 341.4805 |
| Q9Y305 | I05cd8 | Mitochondrial Acyl-CoA Thioeste [symbol] | 447.9229 | 475 | 341.5085 |
| P17096 | L14ef6 | high-mobility group (nonhistone [symbol] | 393.4158 | 266 | 341.5301 |
| P28677 | E10cd3 | visinin-like 1 | 235.283 | 241 | 341.6123 |
| Q9UBX5 | K04cd6 | fibulin 5 | 79.52593 | 108 | 341.7638 |
| Q9Y304 | E12ef2 | ovarian cancer overexpressed 1 | 326.0044 | 238 | 341.8947 |
| O15379 | L16ef6 | histone deacetylase 3 | 198.0005 | 380 | 342.3529 |
| Q02790 | H16ab5 | FK506 binding protein 4 (59 kD) | 323.5993 | 240 | 343.4254 |
| Q9H240 | K12ef5 | Notch homolog 2 (Drosophila) | 270.5904 | 566 | 343.4339 |
| O76095 | P22cd6 | jumping translocation breakpoint | 252.8696 | 227 | 343.482 |
| Q9Y5Q8 | P07cd7 | general transcription factor IIIC; [symbol] | 545.6403 | 324 | 343.9958 |
| Q9NNW5 | H06ef8 | WD repeat domain 6 | 254.1607 | 369 | 344.0253 |
| P01121 | C09ef6 | ras homolog gene family; memb [symbol] | 241.7544 | 304 | 344.0732 |
| Q9H0V7 | K18gh8 | hypothetical protein DKFZp564O | 568.248 | 304 | 344.2985 |
| O60664 | P02cd5 | cargo selection protein (mannos [symbol] | 520.0919 | 232 | 344.5523 |
| P56554 | O15cd3 | ubiquitin-conjugating enzyme E2 | 374.4383 | 417 | 345.0269 |
| O00330 | O06cd3 | Pyruvate dehydrogenase comple [symbol] | 345.9372 | 430 | 345.2213 |
| O60784 | N17cd5 | target of myb1 (chicken) | 364.3098 | 317 | 345.2431 |
| Q9NQZ0 | H03ef4 | CMP-N-acetylneuraminic acid sy [symbol] | 788.5176 | 339 | 345.3498 |
| P52790 | A04ab5 | hexokinase 3 (white cell) | 260.6034 | 430 | 345.4621 |
| P52815 | B10cd1 | mitochondrial ribosomal protein [symbol] | 234.3191 | 277 | 345.7776 |
| O14983 | O10ef1 | ATPase; Ca++ transporting; card | 167.4068 | 223 | 346.1734 |
| P04259 | D01ab7 | keratin 6B | 534.1837 | 405 | 347.6543 |
| P16455 | M04ef6 | O-6-methylguanine-DNA methylt [symbol] | 299.0887 | 307 | 348.0545 |
| Q9Y5L9 | N04cd6 | Snf2-related CBP activator protei [symbol] | 285.2875 | 187 | 348.0925 |
| P41214 | A16gh1 | ligatin | 237.0742 | 307 | 348.5538 |
| Q9H6U4 | P13gh5 | hypothetical protein FLJ21865 | 419.2747 | 175 | 348.8375 |
| Q15393 | L10ef7 | splicing factor 3b; subunit 3; 130 [symbol] | 242.1115 | 239 | 349.6571 |
| P55055 | C04gh1 | nuclear receptor subfamily 1; gro [symbol] | 325.6291 | 368 | 349.8393 |
| P51805 | M03ef4 | SEX gene | 303.0664 | 383 | 350.6414 |
| O75815 | G23ab3 | breast cancer anti-estrogen resis [symbol] | 249.1903 | 293 | 350.9571 |
| Q9Y2Y9 | F19ef2 | Kruppel-like factor 13 | 241.1753 | 366 | 351.0501 |
| Q99633 | D04cd3 | pre-mRNA processing factor 1 [symbol] | 405.046 | 348 | 351.3478 |
| P05155 | C08ab3 | serine (or cysteine) proteinase in | 303.3386 | 334 | 351.5074 |
| Q9UN50 | O16cd7 | EAP30 subunit of ELL complex | 321.9045 | 326 | 351.5535 |
| Q14011 | E11ab4 | cold inducible RNA binding prote [symbol] | 292.5107 | 412 | 351.6685 |
| O75807 | M02ef3 | protein phosphatase 1; regulator | 371.7509 | 652 | 352.5048 |
| Q14457 | K19ab3 | beclin 1 (coiled-coil; myosin-like | 368.4142 | 678 | 352.9542 |
| Q02818 | J06ef6 | nucleobindin 1 | 330.8747 | 213 | 353.1168 |
| O15235 | L10gh1 | mitochondrial ribosomal protein [symbol] | 355.1234 | 467 | 353.2438 |
| Q9Y3E0 | A02ef2 | CGI-141 protein | 152.8668 | 320 | 353.6986 |
| Q9NQZ5 | H19gh4 | START domain containing 7 | 277.2941 | 280 | 353.7264 |
| Q9UHW4 | D24ef8 | protein × 0001 | 266.9848 | 291 | 353.7644 |
| P82914 | P04gh7 | mitochondrial ribosomal protein [symbol] | 444.1517 | 417 | 354.4965 |
| P25787 | L11ef7 | proteasome (prosome; macropal [symbol] | 432.9379 | 526 | 355.1116 |
| P26447 | D02cd1 | S100 calcium binding protein A4 | 198.7401 | 273 | 355.1396 |
| Q9Y605 | P02gh8 | T-cell activation protein | 411.1148 | 356 | 355.1531 |
| Q14776 | K14cd4 | leucine-zipper-like transcriptiona [symbol] | 202.0365 | 273 | 355.4522 |
| P30837 | C02ab2 | aldehyde dehydrogenase 1 famil [symbol] | 631.0407 | 451 | 355.7998 |
| P09668 | L03ef7 | cathepsin H | 460.8488 | 564 | 356.7735 |
| Q02313 | J02gh6 | Krueppel-related zinc finger prot [symbol] | 402.289 | 391 | 356.9162 |
| Q13148 | H11cd7 | TAR DNA binding protein | 322.5816 | 353 | 357.1404 |
| Q16645 | K24ab5 | FK506 binding protein 1B (12.6 [symbol] | 216.7704 | 486 | 357.2698 |
| | N02gh8 | protein phosphatase 1; regulator | 77.52993 | 71.1 | 357.2714 |
| P57739 | F18gh1 | claudin 2 | 188.162 | 238 | 357.443 |
| O60568 | F13cd4 | procollagen-lysine; 2-oxoglutarat | 332.5306 | 269 | 357.5594 |
| Q9HAU4 | N11gh5 | E3 ubiquitin ligase SMURF2 | 425.5948 | 665 | 357.6911 |
| P49914 | J01cd6 | 5; 10-methenyltetrahydrofolate sy [symbol] | 340.2344 | 238 | 358.0826 |
| O60510 | E11cd8 | atrophin-1 interacting protein 1; [symbol] | 171.9425 | 265 | 358.342 |
| Q9BX68 | L15gh8 | histidine triad nucleotide binding | 207.0703 | 360 | 358.5328 |
| P29084 | B02ab5 | general transcription factor IIE; p | 261.3572 | 225 | 358.821 |
| P09526 | C05ef6 | RAP1B; member of RAS oncoge [symbol] | 320.4156 | 355 | 359.0861 |
| O15471 | P01ef4 | leukocyte immunoglobulin-like re [symbol] | 219.8277 | 260 | 359.2909 |
| Q9UEE9 | K03ab3 | craniofacial development protein | 124.0304 | 245 | 359.5047 |
| Q15011 | L03ef3 | homocysteine-inducible; endopla [symbol] | 352.4459 | 183 | 359.7095 |
| Q9BWG8 | A13gh6 | hypothetical protein MGC2491 | 170.1162 | 203 | 360.534 |
| Q9Y397 | A04ef2 | zinc finger; DHHC domain contai [symbol] | 431.1743 | 495 | 360.6182 |
| Q14596 | H17ab7 | membrane component; chromos [symbol] | 303.2476 | 402 | 361.5265 |
| P23497 | C13cd2 | nuclear antigen Sp100 | 331.8296 | 491 | 361.9825 |
| Q16633 | A24cd1 | POU domain; class 2; associatin [symbol] | 103.1231 | 99.3 | 362.0007 |
| Q99961 | J12cd1 | SH3-domain GRB2-like 1 | 231.086 | 247 | 362.8536 |
| Q07021 | N20ab3 | complement component 1; q sub [symbol] | 471.8742 | 202 | 363.3385 |
| Q16513 | E04cd1 | protein kinase C-like 2 | 546.0111 | 561 | 364.4852 |
| Q9H070 | H10cd8 | adaptor-related protein complex: | 353.2901 | 278 | 364.7541 |
| Q13227 | A16ab5 | G protein pathway suppressor 2 | 557.5311 | 352 | 364.8449 |
| P20339 | M03ef5 | RAB5A; member RAS oncogene | 404.6579 | 664 | 365.7139 |
| Q9NXX4 | G15gh2 | hypothetical protein FLJ20005 | 155.2731 | 246 | 365.9622 |
| Q92900 | N17cd1 | regulator of nonsense transcripts | 328.2926 | 316 | 366.2931 |
| Q9H2K4 | I05gh3 | chromosome 11 open reading fr [symbol] | 198.6548 | 155 | 366.4866 |
| O14917 | E17ef4 | protocadherin 17 | 250.3859 | 311 | 366.6879 |
| Q9NZ88 | J06gh3 | chromosome 6 open reading frar | 199.6701 | 362 | 367.2681 |
| Q9BYC8 | C15gh8 | mitochondrial ribosomal protein [symbol] | 257.9414 | 429 | 367.9727 |
| Q15311 | C21cd7 | ralA binding protein 1 | 295.4648 | 411 | 368.6028 |
| Q9NWE9 | E02gh3 | hypothetical protein FLJ10074 | 455.3747 | 383 | 369.4489 |
| O95571 | G22ef3 | protein expressed in thyroid | 398.0124 | 507 | 369.925 |
| P36941 | A09ab7 | lymphotoxin beta receptor (TNFF | 403.0565 | 454 | 370.0343 |
| P49790 | F11cd5 | nucleoporin 153 kD | 264.6175 | 316 | 370.2793 |
| Q99607 | C06ab4 | E74-like factor 4 (ets domain trar [symbol] | 267.135 | 268 | 371.3515 |
| Q9UIV1 | H05cd8 | CCR4-NOT transcription comple; [symbol] | 201.0959 | 465 | 371.6771 |
| Q9Y3E4 | O13ef2 | CGI-146 protein | 107.5317 | 262 | 371.8333 |
| P19367 | D17ab3 | hexokinase 1 | 512.0647 | 215 | 371.9817 |
| Q14250 | J15cd5 | enigma (LIM domain protein) | 229.1944 | 218 | 371.9927 |
| Q9UL33 | B20ef2 | unknown | 343.1533 | 636 | 372.4427 |
| Q15019 | H03ef5 | neural precursor cell expressed; | 489.0601 | 274 | 372.9279 |
| Q13084 | H05cd6 | melanoma-associated antigen re [symbol] | 421.8259 | 278 | 373.0512 |
| Q9UMR2 | O18cd7 | DEAD/H (Asp-Glu-Ala-Asp/His) [symbol] | 383.654 | 423 | 373.0622 |
| P55855 | H06cd2 | SMT3 suppressor of mif two 3 ho | 454.9011 | 444 | 373.596 |
| Q13155 | G20cd4 | JTV1 gene | 322.6641 | 409 | 373.7416 |
| P78371 | H11cd6 | chaperonin containing TCP1; su [symbol] | 480.5464 | 430 | 374.014 |
| P08173 | P17ab3 | cholinergic receptor; muscarinic | 444.2461 | 514 | 374.4117 |
| O60478 | E02cd2 | transmembrane 7 superfamily m [symbol] | 274.7639 | 438 | 374.61 |
| O60429 | D16gh4 | hypothetical protein FLJ12886 | 142.8322 | 257 | 375.0573 |
| O75935 | O04cd7 | dynactin 3 (p22) | 408.9696 | 432 | 375.1224 |
| Q13147 | J05ab2 | abl-interactor 2 | 299.098 | 249 | 375.379 |
| Q9NPH4 | E14gh3 | LUC7-like (S. cerevisiae) | 207.38 | 302 | 376.3768 |
| Q9UJ96 | C13cd8 | potassium voltage-gated channe [symbol] | 582.717 | 448 | 376.4373 |
| P17081 | C13ef6 | likely ortholog of mouse TC10-al [symbol] | 293.1604 | 417 | 376.4996 |
| P11166 | J05ef5 | solute carrier family 2 (facilitated | 351.7333 | 393 | 376.564 |
| Q14820 | D12ef6 | splicing factor 1 | 382.846 | 408 | 376.7779 |
| Q92935 | I06ab5 | exostoses (multiple)-like 1 | 659.9005 | 276 | 376.8548 |
| P31749 | E20ef6 | v-akt murine thymoma viral onco [symbol] | 385.4669 | 303 | 376.9169 |
| Q13454 | L05cd7 | Putative prostate cancer tumor s [symbol] | 326.9146 | 363 | 377.0852 |
| P35520 | C24ab3 | cystathionine-beta-synthase | 70.85109 | 68.1 | 377.1148 |
| P53602 | M01ab7 | mevalonate (diphospho) decarbc [symbol] | 682.0309 | 178 | 378.114 |
| Q9H7G3 | C04gh6 | mitochondrial ribosomal protein [symbol] | 333.6677 | 207 | 378.1396 |
| Q13123 | C06ab7 | IK cytokine; down-regulator of HI [symbol] | 257.7332 | 439 | 378.1806 |
| O14744 | G14cd6 | SKB1 homolog (S. pombe) | 348.0026 | 410 | 378.4165 |
| Q9Y566 | D16ef8 | SH3 and multiple ankyrin repeat | 209.2687 | 281 | 379.5197 |
| Q9BUJ9 | A03gh7 | Mov10; Moloney leukemia virus | 385.0517 | 472 | 380.308 |
| P51790 | I02ab5 | chloride channel 3 | 330.2251 | 237 | 380.3105 |
| O14547 | J09cd6 | PRP8 pre-mRNA processing fact | 239.0005 | 256 | 380.5886 |
| Q99753 | A05cd7 | cisplatin resistance associated | 257.1873 | 397 | 380.7894 |
| Q9NQC8 | H15gh4 | hypothetical protein LOC58912 | 185.2607 | 276 | 381.0416 |
| O43919 | M01ef6 | linker for activation of T cells | 206.6586 | 105 | 381.2059 |
| P78345 | F15cd6 | ribonuclease P (38 kD) | 321.791 | 530 | 381.5687 |
| P54257 | A03ef5 | huntingtin-associated protein 1 ( [symbol] | 450.2874 | 322 | 382.6029 |
| Q9Y3F4 | G18cd7 | unr-interacting protein | 390.9437 | 335 | 382.6142 |
| P29536 | F14cd7 | leiomodin 1 (smooth muscle) | 343.3731 | 209 | 382.9438 |
| O75925 | K03cd4 | protein inhibitor of activated STA | 423.9992 | 606 | 383.7943 |
| Q9UMX5 | J23ef4 | secreted protein of unknown fun [symbol] | 344.8668 | 306 | 384.5676 |
| Q9UMZ9 | N02cd7 | interleukin enhancer binding fact | 348.7866 | 278 | 384.7729 |
| Q92905 | P03ef6 | COP9 constitutive photomorphog [symbol] | 257.5597 | 392 | 386.1452 |
| Q9NSD7 | L20ef1 | G-protein coupled receptor SALF | 734.6282 | 277 | 386.7193 |
| Q9P2W3 | G01ef3 | guanine nucleotide binding prote [symbol] | 70.71144 | 81.4 | 386.7442 |
| Q9BVI7 | N14gh5 | mitochondrial ribosomal protein [symbol] | 367.7177 | 520 | 386.9198 |
| Q9Y4L1 | B23cd6 | oxygen regulated protein (150 kD | 413.0575 | 372 | 387.5372 |
| Q02363 | F18ab7 | inhibitor of DNA binding 2; domir [symbol] | 484.7132 | 359 | 388.2552 |
| Q9Y5J8 | E06cd8 | translocase of inner mitochondri [symbol] | 440.2763 | 542 | 388.4554 |
| | E22ef1 | G protein-coupled receptor 26 | 499.1316 | 450 | 389.582 |
| Q99501 | N11cd6 | growth arrest-specific 2 like 1 | 359.8065 | 540 | 389.6398 |
| Q00613 | M17ef5 | heat shock transcription factor 1 | 363.3847 | 375 | 389.8695 |
| Q9H5Z1 | H16gh4 | DEAD/H (Asp-Glu-Ala-Asp/His) t [symbol] | 136.5899 | 124 | 389.9835 |
| P09661 | P24cd1 | small nuclear ribonucleoprotein [symbol] | 441.1409 | 115 | 390.2085 |
| P54709 | P05ef5 | ATPase; Na+/K+ transporting; b [symbol] | 425.6647 | 432 | 391.8135 |
| P13164 | M06ef5 | interferon induced transmembra [symbol] | 45.13817 | 256 | 392.1204 |
| Q9Y6E2 | A23ef8 | HSPC028 protein | 329.8857 | 363 | 392.2431 |
| Q9NVG2 | B13gh3 | vacuolar protein sorting 35 (yeas [symbol] | 359.0233 | 557 | 392.2574 |
| P27448 | E10ef5 | MAP/microtubule affinity-regulati [symbol] | 397.7845 | 369 | 392.3918 |
| O15438 | P18cd3 | ATP-binding cassette; sub-family | 618.0409 | 488 | 392.8159 |
| Q9UL48 | I04ef2 | CK2 interacting protein 1; HQ002 | 176.5553 | 215 | 393.4111 |
| O94763 | A11cd4 | RPB5-mediating protein | 333.6215 | 533 | 393.9448 |
| Q13190 | I19cd2 | syntaxin 5A | 179.6148 | 235 | 393.985 |
| Q13122 | D21ef3 | EBNA-2 co-activator (100 kD) | 381.3069 | 375 | 394.5999 |
| O75312 | L01cd4 | zinc finger protein 259 | 348.7542 | 422 | 394.8016 |
| P37837 | D20cd2 | transaldolase 1 | 399.2717 | 326 | 395.693 |
| O75762 | G19cd6 | ankyrin-like with transmembrane | 222.8517 | 364 | 395.7427 |
| Q99969 | K19ef3 | retinoic acid receptor responder [symbol] | 283.5705 | 257 | 396.0287 |
| Q9UNP2 | H15cd2 | solute carrier family 1 (neutral an | 149.1019 | 138 | 396.5014 |
| P56524 | C23cd6 | histone deacetylase 4 | 328.6037 | 303 | 397.1317 |
| Q9H644 | C19gh6 | hypothetical protein MGC5585 | 140.9811 | 228 | 397.2624 |
| Q13627 | O19ab4 | dual-specificity tyrosine-(Y)-phos | 498.484 | 327 | 397.3922 |
| Q03405 | O01ef5 | plasminogen activator; urokinase | 500.1391 | 396 | 398.2456 |
| Q9NPQ8 | L18gh4 | likely ortholog of mouse synemb [symbol] | 372.8251 | 443 | 398.4083 |
| Q15375 | A10ab5 | EphA7 | 243.0574 | 290 | 398.5414 |
| P05114 | G04ab7 | high-mobility group (nonhistone [symbol] | 196.8334 | 278 | 398.7327 |
| Q9UKU4 | C13ef2 | retinal short-chain dehydrogenas | 370.8225 | 497 | 399.4413 |
| P08195 | E21ab7 | solute carrier family 3 (activators | 442.5453 | 345 | 399.5678 |
| Q15545 | J11cd2 | TAF7 RNA polymerase II; TATA | 400.689 | 526 | 401.1308 |
| | D24gh8 | hypothetical protein FLJ14511 | 246.3293 | 225 | 401.3816 |
| Q05397 | L20ef5 | PTK2 protein tyrosine kinase 2 | 389.8718 | 440 | 401.4713 |
| Q9Y6G1 | C21ef8 | PTD011 protein | 434.6906 | 483 | 401.5119 |
| P41212 | O23ef5 | ets variant gene 6 (TEL oncogen | 315.7331 | 502 | 401.8428 |
| O14737 | G05cd5 | programmed cell death 5 | 498.729 | 430 | 402.3091 |
| Q9UHB9 | A10ef3 | signal recognition particle 68 kD | 212.5357 | 247 | 402.3276 |
| Q96D21 | C20ef8 | RASD family; member 2 | 395.909 | 486 | 402.4304 |
| Q9Y4I1 | J05ef1 | myosin VA (heavy polypeptide 1 [symbol] | 371.5954 | 353 | 402.453 |
| Q9Y5L4 | L14ef7 | translocase of inner mitochondri [symbol] | 291.7019 | 369 | 404.0738 |
| O75439 | N22cd4 | peptidase (mitochondiral proces [symbol] | 512.9004 | 184 | 404.5715 |
| P55010 | K22ab4 | eukaryotic translation initiation fa | 427.4989 | 296 | 405.4996 |
| Q9BY44 | G10gh8 | CDA02 protein | 453.6064 | 401 | 405.956 |
| Q13164 | B21ef5 | mitogen-activated protein kinase | 209.1026 | 256 | 406.234 |
| P53667 | P08ab6 | LIM domain kinase 1 | 359.4655 | 392 | 406.5183 |
| Q9UNH6 | F05ef2 | sorting nexin 7 | 479.6898 | 396 | 406.7812 |
| O00258 | K17cd4 | tryptophan rich basic protein | 339.3172 | 500 | 407.1504 |
| Q9Y3I0 | I12ef3 | hypothetical protein | 458.2693 | 546 | 407.2408 |
| Q9Y368 | D13cd8 | parvin; beta | 114.232 | 159 | 407.3882 |
| P14866 | H13ab6 | heterogeneous nuclear ribonucle | 442.5489 | 274 | 407.4025 |
| Q13885 | K01cd3 | tubulin; beta polypeptide | 441.2669 | 413 | 407.8683 |
| O43765 | J06cd1 | small glutamine-rich tetratricope [symbol] | 378.9705 | 438 | 408.6453 |
| O14632 | B12ef4 | homeodomain interacting protein | 394.449 | 535 | 408.8773 |
| P31939 | L10ab2 | 5-aminoimidazole-4-carboxamid [symbol] | 501.3028 | 386 | 409.3069 |
| P28331 | P18ab8 | NADH dehydrogenase (ubiquino [symbol] | 490.4347 | 417 | 409.3283 |
| P41970 | P05ef6 | ELK3; ETS-domain protein (SRF | 205.9453 | 359 | 410.8037 |
| Q92973 | J22ab6 | karyopherin (importin) beta 2 | 393.6551 | 234 | 410.9425 |
| P36507 | J10ef5 | mitogen-activated protein kinase | 395.22 | 437 | 411.4052 |
| Q16512 | F21ab8 | protein kinase C-like 1 | 456.2727 | 486 | 411.8245 |
| O43447 | M08cd6 | peptidyl prolyl isomerase H (cyc [symbol] | 179.441 | 222 | 411.9165 |
| P30040 | M13ef3 | chromosome 12 open reading fr [symbol] | 346.6792 | 296 | 412.2064 |
| O95399 | C19cd7 | urotensin 2 | 452.0721 | 366 | 413.1451 |
| | M01ab3 | UDP-Gal:betaGal beta 1; 3-galac | 499.9529 | 290 | 413.3454 |
| Q12792 | N11ab8 | protein tyrosine kinase 9 | 476.3952 | 364 | 413.4208 |
| Q14318 | J02cd7 | FK506 binding protein 8 (38 kD) | 461.2322 | 432 | 413.4238 |
| O75955 | N10cd5 | fiotillin 1 | 304.6674 | 310 | 415.4364 |
| Q9BVY8 | B09gh7 | hypothetical protein MGC5499 | 490.2382 | 417 | 416.02 |
| Q16288 | O19ef5 | neurotrophic tyrosine kinase; rec | 126.7467 | 188 | 416.4128 |
| O75380 | J20ab8 | NADH dehydrogenase (ubiquino [symbol] | 449.5778 | 413 | 416.4836 |
| Q9BY51 | B15gh8 | hypothetical protein GL009 | 429.1998 | 354 | 417.3575 |
| Q9BVK3 | E19gh6 | hypothetical protein MGC2463 | 462.5828 | 387 | 417.7046 |
| Q9P0N3 | A24ef2 | hypothetical protein FLJ10597 | 137.7487 | 289 | 417.999 |
| P24534 | D03ab4 | eukaryotic translation elongation | 347.2405 | 328 | 418.0152 |
| P49336 | C01ab4 | cyclin-dependent kinase 8 | 426.3751 | 281 | 418.121 |
| P34130 | P16cd2 | neurotrophin 5 (neurotrophin 4/5 [symbol] | 327.4146 | 223 | 418.6011 |
| O75425 | P06gh5 | hypothetical protein AF053356_ [symbol] | 340.4357 | 339 | 419.3637 |
| Q15293 | F16ab8 | reticulocalbin 1; EF-hand calciu [symbol] | 241.6383 | 250 | 420.5597 |
| O95376 | D08ab2 | ariadne homolog 2 (Drosophila) | 254.1122 | 322 | 420.8229 |
| O75489 | J16ab8 | NADH dehydrogenase (ubiquino [symbol] | 375.7034 | 327 | 422.3772 |
| P28482 | B15ef5 | mitogen-activated protein kinase | 493.835 | 452 | 422.4625 |
| P20823 | B09cd1 | transcription factor 1; hepatic; LF | 322.4782 | 527 | 423.4405 |
| O15250 | I04cd4 | X-prolyl aminopeptidase (amino [symbol] | 524.8188 | 542 | 423.7387 |
| P55769 | P22ab8 | NHP2 non-histone chormosome [symbol] | 137.3557 | 479 | 424.0031 |
| O94817 | N21ab2 | APG12 autophagy 12-like (S. ce [symbol] | 631.6055 | 402 | 424.3577 |
| O75352 | K08cd5 | mannose-P-dolichol utilization d [symbol] | 272.444 | 523 | 424.8266 |
| Q16585 | I16cd1 | sarcoglycan; beta (43 kD dystrop [symbol] | 256.2113 | 293 | 425.1172 |
| Q9H7X7 | B16gh5 | hypothetical protein FLJ14117 | 261.9578 | 463 | 425.3725 |
| O15173 | I16cd6 | progesterone receptor membrane | 295.0481 | 244 | 425.8728 |
| Q9GZX9 | A16gh4 | twisted gastrulation | 84.61732 | 147 | 426.1124 |
| Q16594 | M09cd2 | TAF9 RNA polymerase II; TATA | 171.3507 | 520 | 426.7606 |
| O00479 | O17gh1 | high-mobility group (nonhistone [symbol] | 265.695 | 354 | 426.7642 |
| P54760 | C22ab5 | EphB4 | 528.7066 | 413 | 427.0543 |
| P25490 | F06ef6 | YY1 transcription factor | 481.2844 | 530 | 427.184 |
| Q9UJY0 | B07ef3 | putative nucleotide binding prote [symbol] | 505.9493 | 431 | 427.3544 |
| Q09666 | A22ef1 | AHNAK nucleoprotein (desmoyol | 392.0671 | 399 | 427.3828 |
| O15305 | P22ab7 | phosphomannomutase 2 | 325.0777 | 480 | 428.3778 |
| O60492 | M09ab4 | 7-dehydrocholestrol reductase | 388.5228 | 213 | 428.5923 |
| Q9UND3 | G11cd6 | nuclear pore complex interacting | 280.8175 | 312 | 428.5952 |
| Q9HB15 | M15gh5 | potassium channel: subfamily K: | 473.5589 | 408 | 428.9306 |
| Q9Y3B4 | H23ef2 | CGI-110 protein | 290.9669 | 331 | 429.1497 |
| Q14107 | F05ef5 | D123 gene product | 387.0465 | 405 | 429.9331 |
| P52435 | A22cd1 | polymerase (RNA) II (DNA direct | 527.0446 | 461 | 429.9488 |
| Q9Y262 | L11ef2 | eukaryotic translation initiation fa [symbol] | 591.5767 | 311 | 430.6788 |
| P10163 | L20cd2 | proline-rich protein BstNI subfam [symbol] | 521.4154 | 251 | 431.1299 |
| P38378 | F01cd8 | protein transport protein SEC61 [symbol] | 635.5838 | 369 | 431.9958 |
| P48735 | B06ab6 | isocitrate dehydrogenase 2 (NA [symbol] | 600.2178 | 338 | 432.1616 |
| Q9NPF5 | F02gh4 | DNA methyltransferase 1-associ [symbol] | 451.5343 | 382 | 432.3473 |
| O14616 | K22cd4 | zinc finger protein-like 1 | 194.5871 | 348 | 432.4885 |
| Q00839 | A12ab7 | heterogeneous nuclear ribonucle [symbol] | 416.5145 | 410 | 432.5188 |
| O95563 | J15ef8 | DKFZP564B167 protein | 390.848 | 389 | 432.8082 |
| Q92974 | I03cd6 | rho/rac guanine nucleotide exch [symbol] | 142.3392 | 124 | 433.2386 |
| Q9UEE5 | M07cd5 | serine/threonine kinase 17a (apc [symbol] | 397.8599 | 472 | 433.4221 |
| Q9NX81 | O19gh3 | BTB (POZ) domain containing 2 | 175.3887 | 206 | 433.4811 |
| Q9NXF9 | O01gh3 | O-linked mannose beta1; 2-N-ac [symbol] | 477.6375 | 353 | 434.2927 |
| P98171 | F13ef1 | Rho GTPase activating protein 4 | 368.9887 | 231 | 435.3585 |
| P56192 | I14ab7 | methionine-tRNA synthetase | 263.0076 | 295 | 435.894 |
| O95411 | K09cd5 | TGFB1-Induced anti-apoptotic fa [symbol] | 233.3926 | 310 | 436.2684 |
| P09132 | E23cd2 | signal recognition particle 19 kD | 227.8193 | 595 | 436.5994 |
| Q92537 | O24gh1 | KIAA0247 gene product | 291.1054 | 232 | 437.1141 |
| P49761 | G03ab4 | CDC-like kinase 3 | 458.9286 | 562 | 437.5601 |
| Q9HDC4 | O19gh4 | junctophilin 3 | 346.7829 | 388 | 439.282 |
| P98160 | A13ef7 | heparan sulfate proteoglycan 2 ( [symbol] | 505.6962 | 372 | 439.9544 |
| Q9NQV6 | O22gh4 | PR domain containing 10 | 369.157 | 514 | 440.5347 |
| Q9NRX6 | J09gh4 | protein × 013 | 324.5306 | 345 | 440.8995 |
| O76084 | A13cd4 | catenin (cadherin-associated pro [symbol] | 471.1305 | 211 | 441.7617 |
| P23197 | E19cd7 | chromobox homolog 1 (HP1 beta | 480.8183 | 362 | 441.9595 |
| Q99439 | E10ab6 | calponin 2 | 429.1961 | 304 | 442.4148 |
| O14656 | O04ef6 | dystonia 1: torsion (autosomal d [symbol] | 415.3893 | 533 | 442.9429 |
| Q9NPJ3 | P04gh4 | uncharacterized hypothalamus p [symbol] | 411.0811 | 430 | 443.2575 |
| P41226 | B10ef7 | ubiquitin-activating enzyme E1- [symbol] | 298.6597 | 342 | 443.334 |
| Q9Y478 | C24cd1 | protein kinase; AMP-activated; b [symbol] | 306.0158 | 165 | 443.5049 |
| | P06cd8 | nodal homolog (mouse) | 349.4202 | 252 | 443.7188 |
| Q12904 | O06ef7 | small inducible cytokine subfamil [symbol] | 374.6683 | 459 | 445.5171 |
| P20264 | C02cd1 | POU domain; class 3; transcription | 458.6994 | 302 | 447.8929 |
| Q15428 | B11cd4 | splicing factor 3a; subunit 2; 66 kD | 284.0454 | 334 | 448.6652 |
| P49771 | G06ef6 | fms-related tyrosine kinase 3 liga [symbol] | 449.6827 | 417 | 452.1329 |
| P49005 | A18cd1 | polymerase (DNA directed); delt [symbol] | 363.5225 | 232 | 452.6832 |
| Q99879 | M14gh6 | H2B histone family; member E | 325.9523 | 483 | 452.8012 |
| P49821 | G20cd1 | NADH dehydrogenase (ubiquino [symbol] | 511.5488 | 392 | 454.6223 |
| P10644 | H22ef5 | protein kinase; cAMP-dependent | 420.8225 | 518 | 454.8256 |
| Q9Y5J4 | L17gh6 | pyrroline 5-carboxylate reductase | 145.6987 | 303 | 455.1667 |
| P31040 | K02cd2 | succinate dehydrogenase compl [symbol] | 671.3369 | 351 | 456.2212 |
| Q9UHA2 | F04ef8 | synovial sarcoma translocation g [symbol] | 438.6951 | 480 | 456.469 |
| Q9UBF6 | C02ef3 | ring finger protein 7 | 315.6541 | 400 | 456.9368 |
| Q9Y5S2 | C21cd6 | CDC42 binding protein kinase b [symbol] | 386.9683 | 447 | 456.9709 |
| P20809 | H07ef7 | interleukin 11 | 399.048 | 140 | 458.1544 |
| Q13526 | F21ef5 | protein (peptidyl-prolyl cis/trans [symbol] | 333.4194 | 460 | 458.2237 |
| Q9NWQ9 | H01gh2 | hypothetical protein FLJ20671 | 430.7122 | 577 | 458.2293 |
| Q9Y6J0 | E05cd8 | calcineurin binding protein 1 | 474.3964 | 117 | 458.7489 |
| Q9Y4Z6 | B13cd7 | vacuolar protein sorting 45A (ye [symbol] | 115.1679 | 276 | 458.8586 |
| Q01433 | G02ab2 | adenosine monophosphate dean | 352.5251 | 390 | 459.2935 |
| O43181 | M07ab8 | NADH dehydrogenase (ubiquino [symbol] | 417.4586 | 432 | 460.2425 |
| Q9NWT5 | D15gh2 | hypothetical protein FLJ20618 | 354.3599 | 523 | 460.3326 |
| O75431 | P19cd6 | metaxin 2 | 282.4791 | 443 | 460.352 |
| P09543 | A18gh7 | 2′;3′-cyclic nucleotide 3′ phospho [symbol] | 258.2862 | 510 | 462.033 |
| Q9BTE1 | B05gh8 | dynactin 4 | 562.6142 | 460 | 462.4054 |
| P16152 | H14ab5 | carbonyl reductase 1 | 234.3184 | 240 | 463.6916 |
| O15121 | D06cd3 | degenerative spermatocyte homo [symbol] | 264.7024 | 270 | 464.4612 |
| O60909 | E09ab3 | UDP-Gal: betaGlcNAc beta 1; 4- [symbol] | 365.8881 | 379 | 464.9697 |
| O75223 | C11gh6 | hypothetical protein MGC3077 | 493.9011 | 276 | 465.7709 |
| Q9BQA1 | K03gh6 | MEP50 protein | 379.1943 | 303 | 466.1997 |
| O43427 | F20cd4 | fibroblast growth factor (acidic) [symbol] | 323.6471 | 325 | 466.8724 |
| Q9BWG6 | A15gh6 | hypothetical protein MGC3180 | 309.3486 | 596 | 468.0924 |
| Q9Y6R2 | M04cd6 | chromosome 4 open reading frar [symbol] | 337.7358 | 352 | 469.9502 |
| Q9HBK7 | M07gh4 | NPD007 protein | 367.4893 | 489 | 471.1924 |
| P52758 | E04ef7 | translational inhibitor protein p14 | 324.9333 | 318 | 471.2741 |
| P47897 | E19cd1 | glutaminyl-tRNA synthetase | 400.1718 | 364 | 472.5359 |
| Q9BWQ6 | P20gh5 | hypothetical protein MGC3262 | 288.8083 | 483 | 473.0084 |
| P35325 | H22cd2 | small proline-rich protein 2B | 390.5307 | 584 | 474.2762 |
| Q9HA68 | O23gh5 | hypothetical protein FLJ12154 | 556.3876 | 383 | 474.593 |
| P49366 | P16ab5 | deoxyhypusine synthase | 458.6886 | 481 | 477.1999 |
| Q9UKZ7 | J18ef1 | colon carcinoma related protein | 505.0182 | 450 | 478.4787 |
| P01730 | K15ef5 | CD4 antigen (p55) | 516.4046 | 432 | 478.8033 |
| P20674 | L06cd4 | cytochrome c oxidase subunit Va | 512.4055 | 372 | 480.2708 |
| Q12828 | H02ef6 | far upstream element (FUSE) bin | 462.592 | 312 | 480.5079 |
| Q9Y6A4 | J18ef3 | similar to mouse Git3 or D. malar [symbol] | 359.9248 | 485 | 480.8661 |
| O60888 | B09ef2 | divalent cation tolerant protein C | 298.6981 | 304 | 481.136 |
| O75716 | F08cd3 | serine/threonine kinase 16 | 364.1875 | 379 | 481.1652 |
| P30711 | E18ef7 | glutathione S-transferase theta 1 | 327.8121 | 533 | 481.9461 |
| Q15008 | L03gh1 | KIAAO107 gene product | 475.6485 | 506 | 482.3327 |
| P17174 | N07ab4 | glutamic-oxaloacetic transamina [symbol] | 412.5293 | 224 | 484.4384 |
| P13798 | K16ab2 | N-acylaminoacyl-peptide hydrola | 365.1218 | 351 | 484.44 |
| Q9P2R8 | E15ef4 | Misshapen/NIK-related kinase | 469.5725 | 511 | 484.515 |
| Q9UNT1 | E20cd7 | RAB; member of RAS oncogene | 268.916 | 347 | 484.9919 |
| O75607 | L11cd7 | nucleophosmin/nucleoplasmin; 3 | 350.2387 | 272 | 485.5671 |
| O94925 | B20gh1 | glutaminase | 410.4357 | 471 | 486.8946 |
| O95390 | A06ab3 | growth differentiation factor 11 | 264.606 | 324 | 489.3912 |
| O75956 | K13cd6 | tumor suppressor deleted in oral | 524.962 | 482 | 489.6212 |
| O95573 | I23ab5 | fatty-acid-Coenzyme A ligase; lo [symbol] | 354.1387 | 174 | 489.6455 |
| Q9BXH1 | N23gh6 | Bcl-2 binding component 3 | 493.3887 | 433 | 492.1083 |
| P13662 | L20cd5 | nuclear transport factor 2 | 392.576 | 479 | 492.4942 |
| Q92793 | H05ab5 | CREB binding protein (Rubinstel | 468.559 | 474 | 494.3525 |
| Q9P0H6 | K23ef4 | AD-012 protein | 406.4789 | 469 | 495.1858 |
| Q9P0R3 | C05ef3 | hypothetical protein HSPC213 | 339.0916 | 509 | 495.2656 |
| O75094 | E11gh1 | slit homolog 3 (Drosophila) | 345.9855 | 439 | 496.3812 |
| Q9P011 | P10ef2 | non-canonical ubquitin conjugati [symbol] | 460.4398 | 538 | 497.256 |
| Q92504 | M12cd4 | HLA class II region expressed g [symbol] | 251.7256 | 339 | 497.6138 |
| O95190 | L12cd8 | omithine decarboxylase antizym [symbol] | 421.0592 | 441 | 497.6839 |
| Q9Y230 | N12cd6 | RuvB-like 2 (E. coli) | 342.0841 | 300 | 497.7767 |
| Q9BSG0 | M16gh8 | chromosome 2 open reading frar | 324.5321 | 458 | 497.8462 |
| Q9Y366 | I17ef2 | chromosome 20 open reading fra [symbol] | 278.1968 | 453 | 498.3057 |
| Q14094 | O24ef5 | cyclin I | 407.6242 | 354 | 499.6223 |
| P43403 | H04ef5 | zeta-chain (TCR) associated pro | 396.0583 | 396 | 500.6282 |
| P52803 | H23ef7 | ephrin-A5 | 357.5339 | 316 | 501.0718 |
| O95402 | E18cd5 | cofactor required for Sp1 transcri [symbol] | 106.0683 | 58.7 | 501.9814 |
| O00233 | L23ab8 | proteasome (prosome; macropali [symbol] | 581.8818 | 399 | 502.2082 |
| Q9H1K6 | O18gh7 | mesoderm development candidar | 432.735 | 560 | 502.7516 |
| Q9BXL6 | K15gh6 | caspase recruitment domain prot | 425.1251 | 523 | 502.8514 |
| Q9H173 | H03gh5 | endoplasmic reticulum chaperon [symbol] | 456.5646 | 449 | 503.5156 |
| Q9NV83 | H03gh3 | hypothetical protein FLJ10876 | 423.9423 | 497 | 504.9702 |
| P11926 | A12ab8 | omithine decarboxylase 1 | 591.3922 | 362 | 505.3016 |
| Q04743 | D23ef1 | empty spiracles homolog 2 (Dros | 531.3866 | 311 | 505.7367 |
| Q9BVK2 | G11gh6 | hypothetical protein MGC2840 si | 388.8682 | 383 | 505.9804 |
| O43251 | I16ef3 | RNA binding motif protein 9 | 346.6445 | 530 | 507.0674 |
| Q99829 | L13cd4 | copine I | 355.3783 | 268 | 508.4915 |
| O95777 | B10ef2 | U6 snRNA-associated Sm-like pr [symbol] | 391.981 | 311 | 508.8683 |
| Q9UN53 | C19ef2 | calcium binding protein Cab45 pr [symbol] | 327.721 | 407 | 509.3316 |
| P24310 | F03ab4 | cytochrome c oxidase subunit VII | 184.0695 | 288 | 509.4504 |
| Q9H929 | N09gh5 | hypothetical protein FLJ13055 | 387.341 | 500 | 510.1197 |
| P10768 | K19ef1 | esterase D/formylglutathione hyd [symbol] | 596.3068 | 102 | 510.5002 |
| O95287 | D17cd5 | golgi autoantigen; golgin subfam | 346.1135 | 560 | 510.6433 |
| Q01105 | H18cd1 | SET translocation (myeloid leuke [symbol] | 328.6513 | 403 | 511.1477 |
| Q9UK45 | B08ef2 | U6 snRNA-associated Sm-like pr | 388.4486 | 551 | 511.9386 |
| P35611 | I17ab2 | adducin 1 (alpha) | 435.0349 | 519 | 512.6447 |
| O14681 | K22cd5 | etoposide-induced mRNA | 451.5599 | 324 | 516.5292 |
| P80303 | A05cd1 | nucleobindin 2 | 296.1418 | 236 | 516.7037 |
| Q9NWC0 | N19gh2 | F-box only protein 4 | 434.5394 | 477 | 517.7728 |
| P00367 | B06ab5 | glutamate dehydrogenase 1 | 360.6 | 303 | 518.2525 |
| O00469 | I09ab8 | procollagen-lysine; 2-oxoglutarat | 735.8327 | 238 | 518.6446 |
| Q9UI04 | M18ef4 | mitochondrial ribosomal protein I | 364.1383 | 336 | 520.377 |
| P20338 | N08ab8 | RAB4A; member RAS oncogene | 487.9427 | 457 | 520.7608 |
| Q9HCU8 | I19gh4 | polymerase (DNA-directed); delt [symbol] | 513.52 | 431 | 522.5153 |
| P00519 | I06ef5 | v-abl Abelson murine leukemia v | 232.4636 | 301 | 522.7624 |
| Q13880 | O02cd5 | brain and reproductive organ-ex [symbol] | 572.4849 | 263 | 523.9906 |
| P25789 | L13ef7 | proteasome (prosome; macropali | 436.5895 | 534 | 526.397 |
| O43805 | J16cd3 | Sjogren's syndrome nuclear auto | 368.0123 | 539 | 526.5215 |
| O75817 | A13cd6 | POP7 (processing of precursor; | 375.1187 | 433 | 526.6432 |
| Q9UBI1 | N11cd7 | BUP protein | 337.1018 | 428 | 526.9646 |
| P03950 | G12ab2 | angiogenin; ribonuclease; RNase | 395.0198 | 200 | 527.7882 |
| P78423 | F20cd1 | small inducible cytokine subfamily | 201.965 | 747 | 530.2357 |
| Q9P1D0 | E07gh4 | hypothetical protein PRO2533 | 58.04051 | 73.3 | 531.5331 |
| P03999 | K05ab3 | opsin 1 (cone pigments); short-w | 116.5093 | 37.9 | 532.8048 |
| O95793 | M22cd2 | staufen; RNA binding protein (Dr | 417.7687 | 498 | 534.8626 |
| P22750 | M05ef2 | RAB4B; member RAS oncogene | 297.3584 | 346 | 538.4665 |
| | B06gh8 | ubiquitin UBF-fi | 431.0731 | 472 | 540.8482 |
| Q9NRX8 | J15gh4 | oxidoreductase UCPA | 168.7047 | 523 | 544.0864 |
| Q9UHQ3 | J20gh4 | stromal cell protein | 469.8784 | 214 | 546.1893 |
| Q96S52 | J22gh8 | phosphatidylinositol glycan; clas [symbol] | 404.9498 | 287 | 546.8906 |
| Q9H3F6 | C24gh8 | MSTP028 protein | 442.9007 | 396 | 549.0707 |
| P37198 | K03cd8 | nucleoporin 62 kD | 428.2961 | 499 | 550.8083 |
| Q9H1D4 | B05gh4 | arginyl aminopeptidase (aminop [symbol] | 430.3273 | 477 | 551.8541 |
| O75350 | K04cd5 | glycoprotein; synaptic 2 | 480.7082 | 436 | 552.8489 |
| Q9P024 | D16ef3 | Huntingtin Interacting protein K | 369.5132 | 480 | 553.5493 |
| Q9Y224 | H21ef2 | CGI-99 protein | 449.089 | 472 | 554.6607 |
| Q13361 | O12cd3 | Microfibril-associated glycoprotel | 194.28 | 354 | 557.066 |
| Q02535 | F01ef1 | inhibitor of DNA binding 3; domir | 84.05787 | 174 | 559.513 |
| O95864 | L24cd4 | fatty acid desaturase 2 | 298.4502 | 113 | 559.6571 |
| P53621 | H16ab4 | coatomer protein complex; subur | 409.2779 | 471 | 560.6313 |
| P35638 | N03ab4 | DNA-damage-inducible transcrip | 322.5942 | 318 | 565.361 |
| P22307 | D22cd1 | sterol carrier protein 2 | 354.3674 | 356 | 569.1558 |
| Q92543 | D15gh1 | KIAA0254 gene product | 328.3511 | 356 | 569.5472 |
| P78330 | N06ab8 | phosphoserine phosphatase | 253.4253 | 220 | 569.9825 |
| Q9UNX3 | G15ef2 | ribosomal protein L26-like 1 | 401.3697 | 445 | 572.3441 |
| P49189 | C10ab2 | aldehyde dehydrogenase 9 famil | 446.252 | 277 | 579.314 |
| Q9ULX0 | H03gh6 | inorganic pyrophosphatase | 488.3446 | 402 | 579.6142 |
| O15353 | H21cd3 | winged-helix nude | 407.9375 | 497 | 580.6191 |
| Q9NRP2 | D21gh4 | DC13 protein | 382.8544 | 421 | 581.5421 |
| Q16280 | E18ef1 | cyclic nucleotide gated channel [symbol] | 464.0608 | 360 | 584.8035 |
| P04183 | C04cd2 | thymidine kinase 1; soluble | 346.7317 | 289 | 588.3101 |
| Q12824 | O17ef5 | SWI/SNF related; matrix associa | 218.8831 | 554 | 590.2326 |
| Q99719 | B09ab8 | peanut-like 1 (Drosophila) | 419.5269 | 410 | 592.2598 |
| P46100 | H07ab3 | alpha thalassemia/mental retard [symbol] | 309.8648 | 167 | 594.3041 |
| Q16610 | O05ab5 | extracellular matrix protein 1 | 238.3172 | 335 | 596.1691 |
| P39656 | E23ab6 | dolichyl-diphosphooligosacchari [symbol] | 433.6719 | 367 | 598.0668 |
| P53814 | G07cd3 | smoothelin | 517.4102 | 269 | 598.16 |
| Q9P0N4 | P24ef1 | hypothetical protein LOC51260 | 609.9142 | 286 | 601.9324 |
| Q9BXZ1 | A02gh7 | peptidylprolyl isomerase (cyclopt | 465.4301 | 362 | 603.8737 |
| Q9Y2Q4 | H19ef8 | HSPC002 protein | 337.5615 | 391 | 604.9187 |
| O15527 | A18ab8 | 8-oxoguanine DNA glycosylase | 330.4852 | 282 | 605.3444 |
| Q04323 | G01ef2 | ORF | 497.159 | 255 | 610.1121 |
| P12004 | G18ab8 | proliferating cell nuclear antigen | 332.1465 | 303 | 612.821 |
| Q99720 | M11cd6 | sigma receptor (SR31747 binding | 363.8609 | 379 | 615.0729 |
| P43307 | G13cd2 | signal sequence receptor; alpha | 226.1566 | 322 | 618.9554 |
| Q9H3Y8 | O05gh6 | chromosome 20 open reading fra [symbol] | 221.2984 | 246 | 619.4944 |
| Q9NRX2 | A08gh5 | mitochondrial ribosomal protein [symbol] | 284.6282 | 305 | 627.9834 |
| Q9P2X0 | L10gh2 | dolichyl-phosphate mannosyltran | 285.542 | 431 | 638.6696 |
| Q9Y5Z8 | E05cd7 | male-specific lethal 3-like 1 (Dro [symbol] | 361.7331 | 394 | 639.7431 |
| P78524 | F01cd2 | suppression of tumorigenicity 5 | 343.4117 | 289 | 643.333 |
| P55145 | F02ab2 | arginine-rich; mutated in early st [symbol] | 413.1582 | 420 | 656.6324 |
| P33551 | O23ab5 | CDC28 protein kinase 1 | 296.3533 | 355 | 671.4891 |
| Q9BVQ0 | E09gh6 | hypothetical protein MGC5363 | 43.30788 | 54.5 | 681.2449 |
| P34897 | D15cd2 | serine hydroxymethyltransferase | 440.9652 | 290 | 685.6584 |
| Q9Y5B4 | L23ef2 | androgen induced protein | 268.4081 | 519 | 685.9395 |
| Q9Y2V5 | B15ef2 | transforming growth factor beta 1 | 249.1479 | 327 | 712.3879 |
| Q13953 | B19cd2 | solute carrier family 12 (potassiu [symbol] | 342.5865 | 428 | 729.2688 |
| Q92686 | I11ef6 | neurogranin (protein kinase C su [symbol] | 330.8792 | 299 | 736.2348 |
| O60565 | J05cd8 | cysteine knot superfamily 1; BMF | 106.6904 | 151 | 781.0154 |
| Q9Y4M4 | I21gh3 | putative UDP-GalNAc:polypeptid [symbol] | 346.098 | 251 | 846.4309 |
| P16581 | E01ef7 | selectin E (endothelial adhesion | 109.3094 | 378 | 875.1666 |
| O14733 | P02ef5 | mitogen-activated protein kinase | 98.83312 | 140 | 888.014 |
| Q16623 | M24cd2 | syntaxin 1A (brain) | 403.2337 | 165 | 926.5022 |
| Q9P1M5 | P12gh3 | WW domain containing oxidored | 38.93 | 19.3 | 1292.23 |
| O60613 | L18cd4 | 15 kDa selenoprotein | 1220.902 | 1110 | 1285.07 |
| Q16698 | M07ab4 | 2:4-dienoyl CoA reductase 1; mit | 469.9997 | 706 | 615.3087 |
| Q9HBA8 | D23gh1 | 24-dehydrocholesterol reductase | 768.0888 | 278 | 626.0195 |
| Q9P0I2 | M13ef4 | 30 kDa protein | 546.352 | 691 | 614.5795 |
| Q9UL53 | P24cd7 | 5′-3′ exoribonuclease 2 | 648.132 | 604 | 430.2235 |
| P49902 | N16cd7 | 5′-nucleotidase; cytosolic II | 1040.523 | 1220 | 440.0817 |
| O95336 | P09cd7 | 6-phosphogluconolactonase | 524.3104 | 755 | 812.5955 |
| Q03393 | A23ab8 | 6-pyruvoyltetrahydropterin synth [symbol] | 358.0591 | 700 | 517.4335 |
| Q13443 | P01ef7 | a disintegrin and metalloproteina | 1306.754 | 481 | 445.3663 |
| O75173 | F15ab2 | a disintegrin-like and metalloprot [symbol] | 786.5151 | 1060 | 1254.804 |
| P51572 | O03gh1 | accessory proteins BAP31/BAP2 | 985.1381 | 889 | 656.6554 |
| P42765 | D03ab2 | acetyl-Coenzyme A acyltransfera | 849.6041 | 1280 | 1204.131 |
| P24666 | E05ab2 | acid phosphatase 1; soluble | 1642.966 | 1970 | 1890.909 |
| Q92688 | D21cd6 | acidic (leucine-rich) nuclear phos [symbol] | 470.8575 | 289 | 835.9103 |
| O14639 | B21ab2 | actin binding LIM protein | 1351.973 | 393 | 358.779 |
| O15143 | F06ab2 | actin related protein 2/3 complex | 696.0033 | 513 | 349.0474 |
| O15509 | F12ab2 | actin related protein 2/3 complex | 909.0832 | 889 | 807.751 |
| O15511 | F14ab2 | actin related protein 2/3 complex | 732.1996 | 619 | 466.1372 |
| P12718 | A08ab6 | actin; gamma 2; smooth muscle; | 585.3438 | 846 | 2095.96 |
| Q9P0I6 | O09ef4 | AD-015 protein | 947.0386 | 837 | 1259.571 |
| O43747 | M13ab2 | adaptor-related protein complex | 1104.332 | 532 | 419.4712 |
| P20172 | A11ab6 | adaptor-related protein complex: | 1286.83 | 1280 | 2285.193 |
| O14617 | H07ab2 | adaptor-related protein complex: | 679.4847 | 495 | 357.0868 |
| P07741 | M24ab2 | adenine phosphoribosyltransfera | 1885.065 | 862 | 823.8455 |
| P00568 | A02ab2 | adenylate kinase 1 | 2404.119 | 399 | 354.3768 |
| Q9U1J7 | I06ef2 | adenylate kinase 3 alpha like | 758.7288 | 1060 | 662.8834 |
| P30566 | M07ab2 | adenylosuccinate lyase | 495.8874 | 622 | 518.4238 |
| Q16186 | M11cd7 | adhesion regulating molecule 1 | 622.3901 | 610 | 574.5888 |
| Q9NVF6 | B19gh3 | ADP-ribosylation factor 1 GTPas | 449.3679 | 623 | 676.1878 |
| P26438 | B04ab2 | ADP-ribosylation factor 6 | 732.1241 | 818 | 876.9277 |
| P40616 | D12ab2 | ADP-ribosylation factor-like 1 | 827.1898 | 1220 | 1141.92 |
| P36404 | D14ab2 | ADP-ribosylation factor-like 2 | 1751.048 | 1550 | 1871.661 |
| P56559 | D20ab2 | ADP-ribosylation factor-like 7 | 485.4562 | 598 | 568.6824 |
| P35368 | K17ab2 | adrenergic; alpha-1B: receptor | 1433.715 | 1510 | 1335.995 |
| P15144 | G24ab2 | alanyl (membrane) aminopeptida | 987.3343 | 222 | 480.1162 |
| P30038 | L09ab2 | aldehyde dehydrogenase 4 famil [symbol] | 1179.797 | 598 | 657.4315 |
| P14550 | L05ab2 | aldo-keto reductase family 1: me [symbol] | 555.464 | 886 | 609.9901 |
| O43488 | L07ab2 | aldo-keto reductase family 7: me [symbol] | 546.409 | 525 | 748.5424 |
| P09972 | C16ab2 | aldolase C; fructose-bisphospha [symbol] | 878.5714 | 1050 | 947.5062 |
| O43590 | G12ef4 | alpha-actinin-2-associated LIM p | 776.3757 | 1070 | 501.1993 |
| Q08117 | M19ab2 | amino-terminal enhancer of split | 1779.707 | 1430 | 1219.695 |
| Q13438 | G05cd7 | amplified in osteosarcoma | 658.0513 | 638 | 802.3407 |
| O96018 | M01gh1 | amyloid beta (A4) precursor prot [symbol] | 762.3898 | 750 | 1117.682 |
| Q13564 | P11ab2 | amyloid beta precursor protein bi [symbol] | 587.5144 | 664 | 786.6426 |
| Q9Y679 | B08cd7 | ancient ubiquitous protein 1 | 1191.741 | 1270 | 1022.771 |
| Q9UKB4 | B12ef2 | angiomotin like 2 | 523.7253 | 776 | 568.8766 |
| Q13725 | H19ab2 | angiotensin receptor 1 | 1495.332 | 1360 | 1058.942 |
| P08133 | I20ab2 | annexin A6 | 253.0936 | 413 | 879.9474 |
| P20073 | I22ab2 | annexin A7 | 650.1591 | 481 | 420.4945 |
| P03971 | E22ab2 | anti-Mullerian hormone | 1140.812 | 948 | 640.7158 |
| P30041 | O08cd5 | anti-oxidant protein 2 (non-seleni [symbol] | 1248.922 | 1160 | 3067.945 |
| P27695 | K06ef6 | APEX nuclease (multifunctional [symbol] | 665.9854 | 653 | 835.6897 |
| Q9Y4J7 | P01ab2 | apoptosis inhibitor 5 | 1850.534 | 68.4 | 147.4186 |
| Q9Y5L7 | L03ef2 | apoptosis related protein APR-3 | 662.9122 | 583 | 1426.489 |
| O94778 | O14ab2 | aquaporin 8 | 1475.036 | 1420 | 1474.307 |
| O15296 | E02ab2 | arachidonate 15-lipoxygenase; s [symbol] | 826.8209 | 607 | 720.2105 |
| P48444 | O18ab2 | archain 1 | 1291.871 | 1200 | 1374.223 |
| P00966 | J24ab2 | argininosuccinate synthetase | 1049.688 | 1600 | 949.4889 |
| P42024 | D15ab2 | ARP1 actin-related protein 1 hon [symbol] | 1054.286 | 1720 | 1777.944 |
| O43681 | J16ab2 | arsA arsenite transporter; ATP-bi [symbol] | 540.3448 | 724 | 595.3348 |
| Q9ULX3 | E02ef4 | ART-4 protein | 728.7274 | 595 | 509.3934 |
| O96030 | H16ab2 | arternin | 881.7849 | 728 | 638.2016 |
| P15289 | F24ab2 | arylsulfatase A | 1639.546 | 1230 | 1012.121 |
| P08243 | J18ab2 | asparagine synthetase | 891.3027 | 643 | 1540.113 |
| O95630 | L17ab2 | associated molecule with the SH [symbol] | 660.3522 | 872 | 729.2383 |
| O95135 | B09ab2 | ataxin 2 related protein | 902.4529 | 345 | 342.3299 |
| O14530 | N15ab2 | ATP binding protein associated v [symbol] | 827.4646 | 1390 | 766.257 |
| P30049 | N20ab2 | ATP synthase; H+ transporting; r [symbol] | 625.0408 | 468 | 441.4691 |
| Q9UII2 | I10ef2 | ATPase inhibitor precursor | 776.4612 | 1220 | 641.2787 |
| Q9Y5K8 | F17ef2 | ATPase; H+ transporting; lysoso [symbol] | 998.0597 | 1670 | 1029.886 |
| O75787 | P15ab2 | ATPase; H+ transporting; lysoso [symbol] | 815.707 | 945 | 1081.688 |
| P05023 | P09ef5 | ATPase; Na+/K+ transporting; al [symbol] | 1299.236 | 517 | 709.0882 |
| P45844 | P13ef5 | ATP-binding cassette; sub-family | 863.8068 | 381 | 606.1642 |
| Q9HAP7 | M08gh5 | baculoviral IAP repeat-containing | 1507.175 | 1740 | 1945.196 |
| O94812 | G09ab3 | BAI1-associated protein 3 | 1073.67 | 983 | 826.6524 |
| P35613 | B14ef7 | basigin (OK blood group) | 603.4821 | 474 | 865.2409 |
| Q9BSR6 | A20gh8 | BBP-like protein 1 | 816.219 | 1320 | 914.1927 |
| O00512 | K01ab3 | B-cell CLL/lymphoma 9 | 1357.15 | 76.7 | 80.36541 |
| Q12983 | E19ab5 | BCL2/adenovirus E18 19 kD inte [symbol] | 763.7046 | 548 | 991.4529 |
| Q92934 | K24ef1 | BCL2-antagonist of cell death | 538.8191 | 727 | 603.5103 |
| Q07817 | E10ef6 | BCL2-like 1 | 2062.673 | 2510 | 1097.372 |
| O43892 | M23ab3 | Bicaudal D homolog 1 (Drosophil [symbol] | 654.9397 | 414 | 548.7409 |
| P53004 | O19ab3 | biliverdin reductase A | 490.0724 | 569 | 512.2807 |
| O60629 | G21ab3 | bladder cancer associated protei [symbol] | 607.9847 | 1180 | 1083.411 |
| Q9HBX3 | N21gh6 | brain and nasopharyngeal carcin | 1672.876 | 54 | 44.02379 |
| P56945 | H23ef3 | breast cancer anti-estrogen resis | 1334.88 | 1300 | 1304.356 |
| O15255 | G21cd6 | CAAX box 1 | 733.6878 | 577 | 1202.182 |
| P12830 | C16ef5 | cadherin 1; type 1; E-cadherin (e [symbol] | 1094.64 | 924 | 489.5012 |
| P19022 | E03ef7 | cadherin 2; type 1; N-cadherin (n [symbol] | 337.5528 | 540 | 747.1198 |
| P22223 | K19ef7 | cadherin 3; type 1; P-cadherin (p [symbol] | 896.8467 | 1200 | 372.3054 |
| P22676 | N07ab5 | calbindin 2; (29 kD; calretinin) | 1781.134 | 1500 | 2116.438 |
| Q99828 | O19gh1 | calcium and integrin binding 1 (c [symbol] | 1130.551 | 1320 | 838.3709 |
| P02593 | I18ab4 | calmodulin 1 (phosphorylase kin [symbol] | 1076.299 | 787 | 983.6302 |
| Q13942 | L01ab5 | calmodulin 3 (phosphorylase kin [symbol] | 902.667 | 464 | 871.1455 |
| P27824 | I05ef1 | calnexin | 1492.644 | 1370 | 1543.709 |
| P17655 | C20ef6 | calpain 2; (m/ll) large subunit | 671.5939 | 837 | 549.4365 |
| P04632 | C22ef6 | calpain; small subunit 1 | 1565.935 | 1650 | 1258.047 |
| Q15417 | P17ab5 | calponin 3; acidic | 911.495 | 603 | 737.1204 |
| Q9UEG5 | D17ef8 | capicua homolog (Drosophila) | 629.0213 | 486 | 439.6052 |
| P52907 | O08ab4 | capping protein (actin filament) n [symbol] | 848.0436 | 684 | 584.7035 |
| Q9GZX3 | N16gh1 | carbohydrate (N-acetylglucosami [symbol] | 793.1054 | 929 | 751.3352 |
| Q16619 | K01ab4 | cardiotrophin 1 | 1478.249 | 1220 | 1559.477 |
| Q9UBD9 | I24cd8 | cardiotrophin-like cytokine; neur [symbol] | 361.4077 | 893 | 489.6592 |
| Q15699 | M16cd4 | cartilage paired-class homeopro [symbol] | 747.0317 | 596 | 1109.834 |
| P48729 | A22ab6 | casein kinase 1; alpha 1 | 690.8551 | 1150 | 902.9474 |
| P48730 | L07ab4 | casein kinase 1; delta | 951.5238 | 1160 | 1111.571 |
| P19138 | L08ef5 | casein kinase 2; alpha 1 polypep [symbol] | 483.6062 | 597 | 461.8036 |
| P49662 | C12ef6 | caspase 4; apoptosis-related cys [symbol] | 2013.957 | 2460 | 1017.425 |
| P21964 | F19ef6 | catechol-O-methyltransferase | 697.5886 | 1170 | 1268.869 |
| P26232 | K01ef7 | catenin (cadherin-associated pro [symbol] | 797.8639 | 717 | 810.257 |
| P35222 | G07ef7 | catenin (cadherin-associated pro [symbol] | 969.252 | 1290 | 1103.619 |
| P07339 | N23ef7 | cathepsin D (lysosomal aspartyl [symbol] | 1253.01 | 947 | 1133.879 |
| Q99967 | C18cd6 | Cbp/p300-interacting transactiva [symbol] | 1293.915 | 1600 | 2245.133 |
| P49715 | N17ef6 | CCAAT/enhancer binding protei [symbol] | 882.9323 | 884 | 1045.45 |
| P53567 | N22ab5 | CCAAT/enhancer binding protei [symbol] | 826.9569 | 1160 | 1584.869 |
| O95627 | B21cd8 | CCR4-NOT transcription comple [symbol] | 606.4629 | 851 | 692.4535 |
| Q04900 | G02cd4 | CD164 antigen; sialomucin | 750.3086 | 570 | 673.7234 |
| P07766 | P07ab3 | CD3E antigen; epsilon polypepti [symbol] | 1808.209 | 1410 | 2643.721 |
| P34810 | A21ab4 | CD68 antigen | 2460.227 | 1100 | 1293.961 |
| P18582 | B21ab5 | CD81 antigen (target of antiprolif [symbol] | 1303.144 | 938 | 1598.276 |
| Q16181 | F09ef5 | CDC10 cell division cycle 10 hon [symbol] | 426.6642 | 443 | 751.3623 |
| Q16543 | H01ef5 | CDC37 cell division cycle 37 hon [symbol] | 1085.897 | 1050 | 1336.155 |
| O14735 | I14cd6 | CDP-diacylglycerol-inositol 3-ph [symbol] | 241.7793 | 1180 | 311.7422 |
| P30260 | F13ef5 | cell division cycle 27 | 1060.812 | 1470 | 1469.824 |
| P49427 | B12ef7 | cell division cycle 34 | 762.5808 | 1300 | 766.6843 |
| P29373 | F01ef7 | cellular retinoic acid binding prot [symbol] | 1226.236 | 803 | 1350.177 |
| P41208 | F09ab5 | centrin; EF-hand protein; 2 | 539.1633 | 672 | 676.8582 |
| O00522 | F05ab4 | cerebral cavemous malformation | 870.4309 | 631 | 723.1482 |
| O15183 | B12cd3 | CGG triplet repeat binding protei [symbol] | 402.949 | 610 | 883.3283 |
| Q9Y2Z5 | D07ef2 | CGI-06 protein | 721.6709 | 674 | 1177.641 |
| Q9Y3C3 | J05ef2 | CGI-120 protein | 1229.537 | 1360 | 2124.304 |
| Q9Y3D0 | J11ef2 | CGI-128 protein | 1592.767 | 2370 | 2011.685 |
| Q9Y3E5 | J17ef2 | CGI-147 protein | 532.425 | 788 | 567.0634 |
| Q9Y3E8 | C08ef2 | CGI-150 protein | 785.276 | 924 | 778.0449 |
| Q9Y320 | A19ef2 | CGI-31 protein | 745.0314 | 932 | 703.7829 |
| Q9Y357 | P16ef1 | CGI-40 protein | 817.7272 | 769 | 418.6337 |
| Q9Y360 | A10ef2 | CGI-45 protein | 786.0567 | 914 | 796.6229 |
| Q9Y512 | B04ef3 | CGI-51 protein | 967.3508 | 859 | 1045.638 |
| Q9Y390 | K21ef2 | CGI-81 protein | 1391.902 | 802 | 1140.01 |
| P40227 | N20ab4 | chaperonin containing TCP1; su [symbol] | 1011.613 | 436 | 592.4944 |
| Q99832 | H07cd6 | chaperonin containing TCP1; su [symbol] | 1190.644 | 1090 | 1548.874 |
| Q9UBR5 | I23ef4 | chemokine-like factor 1 | 1047.63 | 1800 | 1059.437 |
| Q9UHN8 | N05ef4 | chemokine-like factor 1 | 1568.851 | 2030 | 1237.728 |
| P54105 | L07ef5 | chloride channel; nucleotide-sen: | 471.0775 | 487 | 640.6855 |
| Q14781 | F09ef1 | chromobox homolog 2(Pc class I | 795.873 | 749 | 713.815 |
| Q9BXS4 | K12cd5 | chromosome 1 open reading frar [symbol] | 874.0189 | 1370 | 904.3066 |
| Q9NPA0 | H07gh4 | chromosome 11 hypothetical pro [symbol] | 998.8577 | 834 | 858.794 |
| Q9UKR5 | G14cd7 | chromosome 14 open reading fra [symbol] | 1181.962 | 1160 | 1285.11 |
| P56378 | M16cd5 | chromosome 14 open reading fra [symbol] | 503.5233 | 690 | 890.9975 |
| O95433 | B22cd7 | chromosome 14 open reading fra [symbol] | 656.1775 | 563 | 438.9755 |
| Q9NV31 | J23gh3 | chromosome 15 open reading fra [symbol] | 853.8367 | 832 | 861.2221 |
| Q9NVE2 | D09gh3 | chromosome 2 open reading frar [symbol] | 648.49 | 671 | 468.6352 |
| Q9UI05 | L04gh4 | chromosome 20 open reading fra [symbol] | 2518.951 | 2140 | 1201.051 |
| Q9P0A7 | M09ef8 | chromosome 20 open reading fra [symbol] | 581.9098 | 1080 | 1200.665 |
| Q9Y3B1 | H22ef1 | chromosome 20 open reading fra [symbol] | 1691.864 | 1230 | 783.7799 |
| Q9BQ89 | C01gh8 | chromosome 20 open reading fra [symbol] | 1460.875 | 952 | 1221.732 |
| Q96C58 | J19gh7 | chromosome 6 open reading frar [symbol] | 1254.647 | 1080 | 480.9999 |
| Q9Y5Z4 | K12ef3 | chromosome 6 open reading frar [symbol] | 915.3818 | 988 | 372.3119 |
| Q00610 | I12cd5 | clathrin; heavy polypeptide (Hc) | 1639.759 | 1210 | 1437.44 |
| P56749 | F04cd7 | claudin 12 | 2262.278 | 2480 | 1233.472 |
| O43809 | M15cd7 | cleavage and polyadenylation sp | 640.2301 | 503 | 683.3467 |
| O96005 | G05ab4 | cleft lip and palate associated tre [symbol] | 1353.338 | 1170 | 1394.845 |
| Q16740 | E20cd4 | ClpP caseinolytic protease; ATP- | 625.8711 | 528 | 475.0848 |
| P55085 | O24ab5 | coagulation factor II (thrombin) re [symbol] | 829.7537 | 979 | 392.7495 |
| P13726 | B05ab4 | coagulation factor III (thrombopla [symbol] | 1079.841 | 880 | 442.6212 |
| Q9Y678 | D14ef8 | coat protein gamma-cop | 815.7993 | 738 | 1009.756 |
| Q15363 | M11ef3 | coated vesicle membrane protein [symbol] | 706.3054 | 804 | 870.2118 |
| O43513 | N06cd4 | cofactor required for Sp1 transcri [symbol] | 591.8619 | 481 | 798.8706 |
| Q9Y281 | N18cd8 | cofilin 2 (muscle) | 671.9394 | 270 | 1093.534 |
| P04141 | G22gh6 | colony stimulating factor 2 (gran [symbol] | 116.2139 | 1290 | 165.718 |
| P02745 | L06ef1 | complement component 1; q sub [symbol] | 683.4431 | 1650 | 798.2366 |
| P29279 | I16ab4 | connective tissue growth factor | 596.4123 | 537 | 1071.517 |
| O43191 | B02cd3 | COP9 constitutive photomorphog [symbol] | 855.8616 | 1030 | 1302.334 |
| Q99627 | C01cd7 | COP9 homolog | 788.2297 | 968 | 1195.497 |
| O15387 | G21cd7 | COP9 subunit 6 (MOV34 homolo [symbol] | 590.9973 | 382 | 628.5041 |
| Q9ULV4 | K18ef3 | coronin; actin binding protein; 1C | 1594.037 | 1200 | 1362.545 |
| Q9BR76 | I14gh7 | coronin; actin-binding protein; 1E | 708.5653 | 1080 | 690.8568 |
| Q14061 | B22cd5 | COX17 homolog; cytochrome c c | 889.1581 | 1440 | 745.8658 |
| Q9Y6B2 | E02ef8 | CREBBP/EP300 inhibitory protei [symbol] | 946.9513 | 1030 | 1988.725 |
| Q9NY68 | C06gh4 | CTL2 gene | 1381.821 | 804 | 788.5002 |
| P24385 | O04ef5 | cyclin D1 (PRAD1: parathyroid a [symbol] | 1214.611 | 2150 | 1100.478 |
| Q9NXT4 | K09gh2 | cyclin M2 | 1560.659 | 1720 | 1582.768 |
| P01034 | I11ab6 | cystatin C (amyloid angiopathy a [symbol] | 1067.127 | 970 | 1366.396 |
| Q15828 | I13ab4 | cystatin E/M | 967.1669 | 2790 | 451.3932 |
| P01036 | C02ab6 | cyctatin S | 1261.605 | 466 | 1069.756 |
| P52943 | I05ab4 | cysteine-rich protein 2 | 874.1939 | 499 | 1277.819 |
| P13498 | G18ab3 | cytochrome b-245; alpha polyper [symbol] | 708.9954 | 1050 | 359.3156 |
| P00167 | C04ab6 | cytochrome b-5 | 2187.128 | 1480 | 1034.311 |
| P00001 | N24gh6 | cytochrome c | 547.6708 | 442 | 517.6125 |
| P10606 | I24gh6 | cytochrome c oxidase subunit Vb [symbol] | 1364.01 | 2100 | 2087.456 |
| P08574 | P10ab5 | cytochrome c-1 | 667.9346 | 443 | 441.6771 |
| Q99426 | C17gh1 | cytoskeleton-associated protein | 560.9411 | 684 | 777.3269 |
| Q07065 | G13cd7 | cytoskeleton-associated protein | 602.5101 | 575 | 1785.415 |
| Q15038 | L13ef3 | DAZ associated protein 2 | 808.0755 | 890 | 564.7611 |
| Q9H2L4 | B18gh8 | DC32 | 408.0427 | 702 | 780.2878 |
| Q9NPA8 | G22gh7 | DC6 protein | 944.3094 | 1570 | 1514.164 |
| O60231 | H13cd3 | DEAD/H (Asp-Glu-Ala-Asp/His) t [symbol] | 753.8815 | 873 | 799.6561 |
| Q9GZR7 | E02gh4 | DEAD/H (Asp-Glu-Ala-Asp/His) t [symbol] | 803.42 | 984 | 774.3765 |
| P17844 | F04ab4 | DEAD/H (Asp-Glu-Ala-Asp/His) t [symbol] | 1482.642 | 1440 | 1670.104 |
| Q08211 | M03ab4 | DEAD/H (Asp-Glu-Ala-Asp/His) t [symbol] | 907.5859 | 658 | 907.4343 |
| Q9UJV9 | D08ef2 | DEAD-box protein abstrakt | 976.8378 | 1230 | 870.3672 |
| P51398 | G18ef6 | death associated protein 3 | 920.3044 | 981 | 814.3518 |
| Q13437 | G22cd4 | Deleted in split-hand/split-foot 1 [symbol] | 1565.344 | 1500 | 1721.872 |
| O60735 | O06cd6 | dendritic cell protein | 858.6375 | 502 | 467.3432 |
| Q16854 | M06ab5 | deoxyguanosine kinase | 858.1918 | 885 | 989.3036 |
| Q9Y295 | H04ab8 | developmentally regulated GTP I | 736.3786 | 634 | 660.7023 |
| P00387 | F09ab3 | diaphorase (NADH) (cytochrome | 1402.134 | 1030 | 1435.74 |
| Q9UHY9 | I08ef2 | dicarbonyl/L-xylulose reductase | 544.2125 | 740 | 938.7807 |
| P36957 | C06ab6 | dihydrolipoamide S-succinyltrans [symbol] | 625.9037 | 713 | 393.7165 |
| Q99075 | M12ef7 | diphtheria toxin receptor (hepari [symbol] | 721.8431 | 638 | 197.117 |
| Q9UFN1 | H10gh6 | DKFZP564B147 protein | 966.5927 | 947 | 1469.105 |
| O95882 | C13ef8 | DKFZP564C1940 protein | 941.4076 | 865 | 804.639 |
| Q9Y269 | C07ef8 | DKFZP564M082 protein | 675.8991 | 1110 | 1208.543 |
| Q9NWE3 | J11ef8 | DKFZP566C243 protein | 419.5607 | 770 | 625.8483 |
| Q9H6Y7 | H04gh6 | DKFZP566H073 protein | 1390.389 | 1480 | 1191.195 |
| Q9Y3X0 | P23ef8 | DKFZP586M1019 protein | 588.3603 | 505 | 672.4872 |
| O60762 | G19cd4 | dolichyl-phosphate mannosyltran | 407.0059 | 581 | 605.8353 |
| P53805 | B11ab5 | Down syndrome critical region g [symbol] | 370.0907 | 663 | 511.9248 |
| Q13597 | N13ef3 | downregulated in ovarian cancer | 594.6931 | 693 | 623.702 |
| Q9C005 | J05gh8 | dpy-30-like protein | 810.4585 | 1210 | 1174.001 |
| Q13448 | J03cd6 | DR1-associated protein 1 (negati [symbol] | 1240.359 | 1140 | 2382.895 |
| Q9UHF9 | K01ef4 | dual oxidase 2 | 1146.14 | 648 | 316.6462 |
| P51452 | N22ab4 | dual specificity phosphatase 3 (v [symbol] | 649.6813 | 605 | 473.5652 |
| Q14203 | A13ab6 | dynactin 1 (p150; glued homolog | 507.5942 | 529 | 623.6948 |
| Q13561 | D19cd6 | dynactin 2 (p50) | 1565.567 | 1380 | 1173.024 |
| Q13409 | I16ef1 | dynein; cytoplasmic; intermediate | 793.3207 | 604 | 464.9675 |
| Q09472 | C18ab4 | E1A binding protein p300 | 1496.409 | 2120 | 1342.944 |
| | D20gh8 | EAF1 protein | 642.1587 | 652 | 469.6834 |
| P78365 | N19ab4 | early development regulator 2 (p [symbol] | 894.3715 | 1070 | 761.5923 |
| Q99848 | G11cd7 | EBNA1 binding protein 2 | 977.8671 | 611 | 818.6583 |
| Q12805 | A21ab6 | EGF-containing fibulin-like extra [symbol] | 1002.82 | 332 | 458.9563 |
| Q9Y6I3 | D21cd8 | EH domain-binding mitotic phosp [symbol] | 736.8984 | 500 | 629.1845 |
| Q15717 | C04ab4 | ELAV (embryonic lethal; abnorm [symbol] | 850.8493 | 800 | 843.4463 |
| P13804 | I24ab3 | electron-transfer-flavoprotein; alp [symbol] | 918.8391 | 545 | 529.2866 |
| Q14247 | K17ef6 | ems1 sequence (mammary tumo [symbol] | 1135.188 | 1050 | 1002.084 |
| P42892 | O21ab4 | endothelin converting enzyme 1 | 602.4294 | 799 | 809.7071 |
| P30084 | A17ab6 | enoyl Coenzyme A hydratase; sh [symbol] | 802.3114 | 1210 | 741.2226 |
| P07099 | I20ab3 | epoxide hydrolase 1; microsomal | 1231.48 | 1840 | 1198.169 |
| P29692 | H04ab5 | eukaryotic translation elongation | 1329.756 | 744 | 1110.87 |
| P20042 | L05cd4 | eukaryotic translation initiation fa [symbol] | 1489.433 | 603 | 956.276 |
| P41091 | A20ab4 | eukaryotic translation initiation fa [symbol] | 742.5843 | 474 | 478.7249 |
| Q99613 | L16cd3 | eukaryotic translation initiation fa [symbol] | 1140.022 | 853 | 998.7488 |
| P78344 | C02ab4 | eukaryotic translation initiation fa [symbol] | 1562.952 | 1520 | 1177.845 |
| P23588 | A24ab4 | eukaryotic translation initiation fa [symbol] | 1343.019 | 1010 | 1125.825 |
| Q13541 | C03ab5 | eukaryotic translation initiation fa [symbol] | 1655.926 | 1060 | 2293.236 |
| Q13542 | A19ab6 | eukaryotic translation initiation fa [symbol] | 1620.111 | 1850 | 1663.197 |
| O60573 | G16cd5 | eukaryotic translation initiation fa [symbol] | 799.7789 | 883 | 819.3679 |
| Q16394 | K02ab3 | exostoses (multiple) 1 | 513.7526 | 428 | 813.2693 |
| P37268 | C17ab6 | famesyl-diphosphate famesyltra [symbol] | 726.0862 | 455 | 649.0518 |
| P55899 | L22ab5 | Fc fragment of IgG; receptor; tra [symbol] | 450.8629 | 771 | 476.1039 |
| Q9UK73 | P05ef3 | fem-1 homolog b (C. elegans) | 548.2677 | 547 | 530.801 |
| P21333 | G14ab4 | filamin A; alpha (actin binding pr [symbol] | 946.0465 | 675 | 1277.458 |
| P26885 | P07ab4 | FK506 binding protein 2 (13 kD) | 814.6357 | 510 | 661.4593 |
| Q13451 | F15ab4 | FK506 binding protein 5 | 1366.905 | 1110 | 812.8568 |
| O95633 | M01cd6 | follistatin-like 3 (secreted glycop [symbol] | 1312.174 | 1280 | 1092.99 |
| P15407 | A20cd4 | FOS-like antigen 1 | 1696.135 | 1350 | 1218.491 |
| Q14192 | G02ab4 | four and a half LIM domains 2 | 309.9219 | 591 | 1210.635 |
| Q9NZA0 | N17gh6 | fuse-binding protein-interacting [symbol] | 924.2347 | 911 | 1110.468 |
| P35637 | P19ab4 | fusion; derived from t(12; 16) mali [symbol] | 866.3996 | 654 | 1159.792 |
| | E21gh7 | FXYD domain-containing ion tra [symbol] | 942.6126 | 1060 | 802.8679 |
| Q9Y653 | B02cd5 | G protein-coupled receptor 56 | 2010.489 | 2160 | 1501.617 |
| Q9H1C0 | E18gh4 | G protein-coupled receptor 92 | 712.1007 | 750 | 719.7999 |
| P15170 | L13ab4 | G1 to S phase transition 1 | 2042.425 | 1410 | 1109.721 |
| O95166 | D09cd7 | GABA(A) receptor-associated pr [symbol] | 1280.495 | 2210 | 1227.378 |
| O08765 | D21cd7 | GABA(A) receptor-associated pr [symbol] | 1241.27 | 1130 | 918.8337 |
| P16278 | O05ef1 | galactosidase; beta 1 | 539.4222 | 800 | 656.0518 |
| P78537 | J23ab4 | GCN5 general control of amino- [symbol] | 813.3309 | 637 | 919.5196 |
| P31150 | F14ab4 | GDP dissociation inhibitor 1 | 633.6157 | 894 | 640.108 |
| P06396 | B23ab3 | gelsolin (amyloidosis; Finnish typ [symbol] | 330.1567 | 577 | 633.2269 |
| P52657 | E03ab6 | general transcription factor IIA; 2 | 538.6557 | 685 | 700.1857 |
| Q00403 | F08ef6 | general transcription factor IIB | 500.4565 | 791 | 454.8328 |
| P48060 | I23cd7 | glioma pathogenesis-related pro [symbol] | 676.4844 | 692 | 752.7928 |
| P06744 | B19ab3 | glucose phosphate isomerase | 900.9772 | 553 | 726.4749 |
| P30101 | G13ab5 | glucose regulated protein; 58 kD | 1708.658 | 1250 | 2220.105 |
| O43836 | I22ef1 | glutamate receptor; ionotropic; N [symbol] | 1729.777 | 1890 | 1655.131 |
| P15104 | H22ab5 | glutamate-ammonia ligase (gluta [symbol] | 1170.59 | 1040 | 1180.088 |
| Q06210 | A23ab5 | glutamine-fructose-6-phosphate [symbol] | 869.494 | 600 | 863.157 |
| Q9Y3D4 | M19ef2 | glutaredoxin 2 | 888.5406 | 894 | 1099.209 |
| Q9Y2Q3 | C24ef2 | glutathione S-transferase subuni [symbol] | 841.3959 | 346 | 414.7892 |
| P48637 | D01ab3 | glultathione synthatase | 650.8496 | 429 | 488.6663 |
| P78417 | E20cd5 | glutathione-S-transferase like; gl [symbol] | 1983.626 | 2020 | 1805.489 |
| P49840 | J14ef4 | glycogen synthase kinase 3 alph [symbol] | 1057.368 | 1460 | 1247.816 |
| P13224 | F23ab3 | glycoprotein lb (platelet): beta pc [symbol] | 487.4684 | 530 | 653.4727 |
| Q9UBQ7 | L08cd7 | glyoxylate reductase/hydroxypyn [symbol] | 947.0262 | 977 | 997.593 |
| Q9P2T1 | C02ef2 | GMPR2 for guanosine monopho [symbol] | 762.0126 | 1070 | 817.0496 |
| Q9H4A6 | I04gh5 | golgi phosphoprotein 3 (coat-pro [symbol] | 1027.603 | 1210 | 1209.783 |
| P28799 | B19ab5 | granulin | 1611.298 | 1750 | 1238.745 |
| Q14393 | H10ab3 | growth arrest-specific 6 | 484.489 | 563 | 739.1661 |
| P29354 | H06ef5 | growth factor receptor-bound pro [symbol] | 721.5894 | 854 | 663.0587 |
| Q14451 | D20ab4 | growth factor receptor-bound pro [symbol] | 712.3296 | 846 | 636.8962 |
| Q9Y6G2 | E04ef8 | growth hormone inducible trans [symbol] | 1289.256 | 1020 | 974.263 |
| P04899 | J09ab4 | guanine nucleotide binding prote [symbol] | 860.9208 | 1390 | 1748.649 |
| P11016 | M13ef5 | guanine nucleotide binding prote [symbol] | 758.629 | 773 | 836.6295 |
| Q13905 | G09ab6 | guanine nucleotide-releasing fac [symbol] | 410.0949 | 569 | 535.1889 |
| Q16774 | L16ab3 | guanylate kinase 1 | 968.5867 | 1340 | 975.8911 |
| Q9Y649 | K16ef4 | GW128 protein | 1663.305 | 2420 | 1834.174 |
| P07305 | G13ab6 | H1 histone family; member 0 | 1780.353 | 1870 | 1746.555 |
| O75367 | M14cd5 | H2A histone family; member Y | 1065.311 | 837 | 455.5967 |
| P17317 | C24ab6 | H2A histone family; member Z | 591.0013 | 461 | 1570.489 |
| P02278 | M16gh6 | H2B histone family; member G | 586.8773 | 852 | 684.6287 |
| Q9NS37 | A03gh5 | HCF-binding transcription factor | 857.3143 | 717 | 601.7268 |
| O96004 | E02cd5 | heart and neural crest derivatives | 705.5163 | 612 | 621.033 |
| P10809 | M11ef5 | heat shock 60 kD protein 1 (chap [symbol] | 1693.23 | 1020 | 1276.41 |
| P34932 | I11ef1 | heat shock 70 kD protein 4 | 1402.505 | 1610 | 1042.113 |
| P07900 | G18ef7 | heat shock 90 kD protein 1; alpha | 2405.793 | 1430 | 1512.258 |
| P08238 | B20ab7 | heat shock 90 kD protein 1; beta | 2781.462 | 1950 | 979.1145 |
| O75506 | H17ab6 | heat shock factor binding protein | 1132.126 | 1300 | 1081.748 |
| Q9UHG4 | K18ef4 | heme-regulated initiation factor 2 | 413.2841 | 659 | 576.3971 |
| P09105 | J21ab4 | hemoglobin; theta 1 | 702.445 | 484 | 769.2336 |
| O43504 | D23cd6 | hepatitis B virus x interacting pro [symbol] | 1008.655 | 1340 | 949.0116 |
| Q9H2I6 | N10gh5 | hepatitis C virus core-binding pro [symbol] | 676.7297 | 620 | 473.9328 |
| P51858 | K16ef7 | hepatoma-derived growth factor [symbol] | 894.1008 | 1170 | 490.7458 |
| Q04150 | A08ab7 | heterogeneous nuclear ribonucle | 943.3857 | 870 | 1065.597 |
| Q13151 | E15cd7 | heterogeneous nuclear ribonucle | 906.986 | 944 | 1143.552 |
| P22626 | N09ab6 | heterogeneous nuclear ribonucle | 1021.87 | 1010 | 1584.855 |
| P07910 | A10ab7 | heterogeneous nuclear ribonucle | 747.5309 | 718 | 1059.349 |
| O14979 | L09cd5 | heterogeneous nuclear ribonucle | 1057.699 | 1990 | 1646.172 |
| P52597 | G06ab7 | heterogeneous nuclear ribonucle | 1902.939 | 1280 | 1517.274 |
| Q07244 | N13ab6 | heterogeneous nuclear ribonucle | 847.6967 | 667 | 684.4978 |
| Q15584 | P03ab7 | heterogeneous nuclear ribonucle | 725.3651 | 569 | 858.2829 |
| P12081 | J12ab4 | histidyl-tRNA synthetase | 555.5578 | 565 | 424.3904 |
| Q9UBN7 | E11cd6 | histone deacetylase 6 | 685.5336 | 407 | 695.1552 |
| Q13838 | M07cd4 | HLA-B associated transcript 1 | 681.4666 | 604 | 921.7532 |
| Q99873 | H02ef7 | HMT1 hnRNP methyltransferase- | 890.1184 | 836 | 1209.872 |
| Q9H063 | M14gh8 | homolog of yeast MAF1 | 744.9951 | 921 | 1013.86 |
| P51610 | L21ab5 | host cell factor C1 (VP16-access | 583.7478 | 580 | 735.1691 |
| O00165 | I04cd6 | HS1 binding protein | 1482.068 | 1490 | 2425.818 |
| Q9Y2Q5 | P24ef7 | HSPC003 protein | 620.7783 | 1010 | 625.6574 |
| Q9Y2R0 | A05ef8 | HSPC009 protein | 1507.005 | 1720 | 1784.568 |
| Q9Y2T0 | A17ef8 | HSPC022 protein | 453.0964 | 733 | 326.4215 |
| Q9UNZ5 | C17ef8 | HSPC023 protein | 825.3888 | 1310 | 1347.488 |
| Q9P032 | O19ef8 | HSPC125 protein | 558.8905 | 602 | 843.0953 |
| Q9P019 | A06ef8 | HSPC141 protein | 717.9096 | 1700 | 1519.292 |
| Q9P004 | A24ef8 | HSPC160 protein | 1182.006 | 1070 | 545.8523 |
| Q9P003 | C04ef8 | HSPC163 protein | 801.5204 | 1410 | 1148.378 |
| Q9P000 | C06ef8 | HSPC166 protein | 508.2074 | 921 | 523.3806 |
| Q9Y684 | N09ef2 | HSPCO34 protein | 569.9533 | 628 | 797.4819 |
| Q9NRG2 | D01gh4 | HTGN29 protein | 1276.132 | 1470 | 1639.451 |
| Q16775 | G15ab6 | hydroxyacyl glutathione hydrolas | 511.3412 | 676 | 407.0538 |
| P40939 | D07ab3 | hydroxacyl-Coenzyme A dehydr [symbol] | 1814.498 | 1770 | 1617.578 |
| P55084 | D09ab3 | hydroxacyl-Coenzyme A dehydr [symbol] | 539.2691 | 890 | 1218.105 |
| Q14526 | B13ab5 | hypermethylated in cancer 1 | 918.3678 | 904 | 1008.759 |
| Q9UBS2 | A15ef1 | hyperpolarization activated cyclic | 676.9289 | 688 | 520.6835 |
| Q96EW8 | P08gh8 | hypothetical gene ZD52F10 | 727.4433 | 605 | 223.8014 |
| Q9Y2S6 | D01ef2 | hypothetical protein | 1188.199 | 1490 | 1593.015 |
| Q9UKZ1 | I23gh3 | hypothetical protein C40 | 657.2427 | 614 | 506.0272 |
| Q9UJJ9 | D21gh8 | hypothetical protein CAB56184 | 432.491 | 695 | 601.25 |
| O95891 | D08ef8 | hypothetical protein CL25022 | 1675.198 | 2070 | 1477.523 |
| Q9UJI9 | F08gh6 | hypothetical protein DJ328E19.C | 1119.481 | 935 | 1005.691 |
| Q9H0U3 | K20gh8 | hypothetical protein DKFZp564K | 1287.049 | 1080 | 1145.302 |
| Q9NWD8 | N05gh2 | hypothetical protein FLJ10099 | 754.9838 | 758 | 710.8818 |
| Q9NW90 | P21gh2 | hypothetical protein FLJ10211 | 492.5962 | 551 | 601.7235 |
| Q9NW61 | B18gh2 | hypothetical protein FLJ10297 | 772.0358 | 844 | 620.8513 |
| Q9NW16 | G04gh3 | hypothetical protein FLJ10374 | 756.2846 | 543 | 467.9722 |
| Q9NVZ3 | G14gh3 | hypothetical protein FLJ10420 | 437.615 | 702 | 362.923 |
| Q9NVC3 | D23gh3 | hypothetical protein FLJ10815 | 1402.842 | 928 | 598.595 |
| Q9H9K7 | B10gh5 | hypothetical protein FLJ12681 | 446.1454 | 657 | 593.5606 |
| Q9H8H4 | H06gh5 | hypothetical protein FLJ12800 | 1058.023 | 775 | 1421.894 |
| Q9P0R5 | L02ef1 | hypothetical protein FLJ14868 | 702.1841 | 684 | 1117.168 |
| Q9NXQ4 | M07gh3 | hypothetical protein FLJ20113 | 1503.987 | 1520 | 1545.141 |
| Q9NXI2 | M21gh3 | hypothetical protein FLJ20234 | 643.4985 | 877 | 178.0291 |
| Q9NXD5 | E18gh2 | hypothetical protein FLJ20309 | 617.1482 | 1420 | 758.0334 |
| Q9NX64 | I16gh2 | hypothetical protein FLJ20419 | 938.8958 | 825 | 981.6359 |
| Q9NWX1 | A20gh3 | hypothetical protein FLJ20552 | 1366.306 | 1800 | 1533.352 |
| Q9NWT8 | D11gh2 | hypothetical protein FLJ20608 | 980.6383 | 915 | 907.2393 |
| Q9NWM3 | C20gh3 | hypothetical protein FLJ20739 | 381.202 | 593 | 689.893 |
| Q9H7C7 | D05gh5 | hypothetical protein FLJ21044 si | 551.5395 | 708 | 585.1487 |
| Q9H6R6 | J23gh5 | hypothetical protein FLJ21952 | 425.3152 | 396 | 679.1125 |
| Q9H673 | F11gh5 | hypothetical protein FLJ22548 si | 1394.825 | 1220 | 530.5646 |
| O95476 | H15ef8 | hypothetical protein HSA011916 | 1310.097 | 1040 | 1485.684 |
| Q9Y244 | B23ef2 | hypothetical protein HSPC014 | 1668.101 | 1530 | 1243.802 |
| Q9P022 | P04ef2 | hypothetical protein HSPC138 | 666.1765 | 521 | 643.5979 |
| Q9P013 | P06ef2 | hypothetical protein HSPC148 | 916.5214 | 1450 | 1133.279 |
| Q9P009 | P14ef2 | hypothetical protein HSPC155 | 1031.658 | 1330 | 662.2481 |
| Q9P0S9 | A19ef3 | hypothetical protein HSPC194 | 1336.172 | 1570 | 1957.055 |
| Q9NPI0 | A23ef3 | hypothetical protein HSPC196 | 280.0356 | 644 | 672.086 |
| O95887 | C18ef2 | hypothetical protein LOC51061 | 777.5591 | 709 | 551.8354 |
| Q9P0T9 | K02ef2 | hypothetical protein LOC51234 | 1224.056 | 1290 | 1457.544 |
| Q9P0Q1 | K10ef2 | hypothetical protein LOC51248 | 376.8259 | 933 | 1014.441 |
| Q9P0P0 | K20ef2 | hypothetical protein LOC51255 | 1301.286 | 2190 | 1904.375 |
| Q9P0N9 | K22ef2 | hypothetical protein LOC51256 | 540.326 | 374 | 604.1319 |
| Q9NZD9 | M23ef2 | hypothetical protein LOC51323 | 600.1582 | 715 | 506.8479 |
| O75207 | M02gh4 | hypothetical protein LOC57019 | 663.8685 | 644 | 680.7704 |
| Q9BWL0 | L24gh7 | hypothetical protein MGC: 5244; | 569.8856 | 436 | 732.9891 |
| Q9BW61 | C09gh6 | hypothetical protein MGC2594 | 1446.395 | 2460 | 1354.586 |
| Q9BQ49 | K05gh6 | hypothetical protein MGC2747 | 527.9287 | 679 | 677.2851 |
| Q9BQD3 | E17gh6 | hypothetical protein MGC2749 | 841.5499 | 889 | 899.9017 |
| Q9BQ61 | A09gh6 | hypothetical protein MGC2803 | 1234.395 | 2170 | 1980.421 |
| Q9BTV4 | O23gh6 | hypothetical protein MGC3222 | 561.6572 | 502 | 583.5781 |
| Q9BVX2 | C17gh6 | hypothetical protein MGC5576 | 747.2749 | 762 | 604.3725 |
| Q9H773 | I17gh6 | hypothetical protein MGC5627 | 799.182 | 819 | 584.0079 |
| Q9BPX5 | L02gh7 | hypothetical protein similar to ac [symbol] | 583.4076 | 424 | 632.4101 |
| Q9UJX8 | B09ef3 | hypothetical protein; estradiol-in [symbol] | 1184.334 | 948 | 1116.899 |
| P00492 | P12cd6 | hypoxanthine phosphoribosyltra [symbol] | 1210.895 | 233 | 566.0945 |
| P22304 | I04ab6 | iduronate 2-sulfatase (Hunter sy [symbol] | 1197.3 | 1320 | 1095.895 |
| Q03827 | F02ef6 | immediate early protein | 650.6358 | 537 | 536.2705 |
| P12268 | F11ab6 | IMP (inosine monophosphate) d [symbol] | 1254.916 | 567 | 1293.693 |
| Q9Y6K9 | P03cd3 | inhibitor of kappa light polypepti [symbol] | 822.3859 | 788 | 807.6888 |
| Q14573 | F04ab6 | inositol 1;4;5-triphosphate recep [symbol] | 555.9397 | 646 | 627.6143 |
| O15357 | L09ab6 | inositol polyphosphate phosphat [symbol] | 997.7512 | 1150 | 1290.473 |
| O15503 | B05ab7 | insulin induced gene 1 | 472.0202 | 428 | 615.7553 |
| P52945 | L13gh8 | insulin upstream factor 1 | 1437.285 | 3700 | 746.514 |
| P51460 | B07ab7 | insulin-like 3 (Leydig cell) | 1227.459 | 1290 | 1040.851 |
| P46977 | D20ab6 | integral membrane protein 1 | 1693.474 | 1790 | 2514.977 |
| Q13418 | H08ef5 | integrin-linked kinase | 1340.442 | 962 | 1599.336 |
| P48551 | O21ef7 | interferon (alpha; beta and omeg | 605.1613 | 669 | 389.178 |
| P15260 | A02ef7 | interferon gamma receptor 1 | 1165.784 | 2600 | 1179.083 |
| P38484 | D12ef5 | interferon gamma receptor 2 (int [symbol] | 593.6133 | 773 | 550.2678 |
| O14896 | K05ef6 | interferon regulatory factor 6 | 495.9782 | 951 | 406.9438 |
| | G13ef5 | interferon-gamma receptor (IFNG | 616.4461 | 474 | 457.8757 |
| Q14116 | L03ab6 | interleukin 18 (interferon-gamma | 1525.444 | 1610 | 556.9614 |
| O75874 | H11ab7 | isocitrate dehydrogenase 1 (NA [symbol] | 660.9773 | 496 | 904.897 |
| P51553 | I21gh1 | isocitrate dehydrogenase 3 (NA [symbol] | 815.5318 | 899 | 784.1063 |
| P41252 | P23ab6 | isoleucine-tRNA synthetase | 643.6728 | 316 | 992.267 |
| Q9UE89 | J07ef7 | jagged 2 | 657.9935 | 460 | 696.5019 |
| P52292 | J14ab6 | karyopherin alpha 2 (RAG cohor [symbol] | 1105.911 | 1270 | 1670.406 |
| P24390 | E07cd7 | KDEL (Lys-Asp-Glu-Leu) endopl [symbol] | 1556.771 | 1210 | 1411.383 |
| P08779 | D03ab7 | keratin 16 (focal non-epidermoly [symbol] | 815.5106 | 786 | 663.0228 |
| Q04695 | O24ab6 | keratin 17 | 580.3123 | 1180 | 1056.274 |
| Q07666 | B04cd6 | KH domain containing: RNA bind | 1212.816 | 780 | 1103.092 |
| Q14165 | O16gh1 | KIAA0152 gene product | 285.0232 | 348 | 1374.81 |
| Q12765 | F03gh1 | KIAA0193 gene product | 951.658 | 1000 | 981.6527 |
| O43310 | F11gh1 | KIAA0427 gene product | 482.1741 | 589 | 556.0999 |
| O43167 | J03gh1 | KIAA0441 gene product | 1398.244 | 982 | 1102.589 |
| Q9H7D5 | I18gh7 | KIAA1191 protien | 886.4026 | 1930 | 1061.302 |
| Q9HD96 | L03cd8 | LAG1 longevity assurance homol | 1319.272 | 798 | 1016.572 |
| O00182 | P04ab6 | lectin; galactoside-binding: solub | 760.6622 | 1300 | 527.6081 |
| Q99538 | B04ef7 | legumain | 464.5 | 517 | 1105.037 |
| O95751 | G01cd8 | leucine zipper; down-regulated ir [symbol] | 874.8344 | 274 | 970.9239 |
| O15468 | P03ef4 | leukocyte immunoglobulin-like re [symbol] | 826.8073 | 939 | 1120.204 |
| Q9UBB4 | I06cd8 | like mouse brain protein E46 | 877.3997 | 623 | 692.5911 |
| Q9UFW4 | N17ef8 | likely ortholog of rat golgi stackin | 1680.361 | 1700 | 2121.05 |
| Q14847 | P09ab7 | LIM and SH3 protein 1 | 664.6143 | 758 | 895.1972 |
| O00158 | M09ef3 | LIM domain only 4 | 457.4088 | 299 | 1353.355 |
| Q9HAP6 | M16gh5 | Lin-7b protein: likely ortholog of [symbol] | 2114.229 | 1550 | 2175.236 |
| P38571 | K08ab6 | lipase A; lysosomal acid; cholest, | 604.0401 | 587 | 512.8879 |
| Q99732 | I18cd5 | LPS-induced TNF-alpha factor | 680.3876 | 893 | 449.4711 |
| Q9Y4Z1 | O21ef4 | Lsm3 protein | 1641.224 | 1300 | 1596.905 |
| O75896 | D05cd7 | lung cancer candidate | 690.6061 | 772 | 601.3296 |
| Q06643 | O07ef6 | lymphotoxin beta (TNF superfam [symbol] | 405.7368 | 1040 | 298.9254 |
| O95372 | J04ef7 | lysophospholipase II | 480.0562 | 737 | 752.7079 |
| P13473 | N06ab6 | lysosomal-associated membrane | 952.6393 | 2000 | 1277.773 |
| Q08397 | F09ab7 | lysyl oxidase-like 1 | 1391.453 | 1630 | 2243.025 |
| Q15046 | B17ab7 | lysyl-tRNA synthetase | 900.8177 | 556 | 539.813 |
| Q13312 | D17cd3 | MAD1 mitotic arrest deficient-like | 807.7267 | 503 | 574.4266 |
| Q09160 | A05ab6 | major histocompatibility complex; | 1524.076 | 2710 | 1290.783 |
| P40925 | J23ab7 | malate dehydrogenase 1; NAD ( [symbol] | 1144.218 | 788 | 1089.134 |
| P40926 | L01ab7 | malate dehydrogenase 2; NAD ( [symbol] | 1525.837 | 1020 | 1212.544 |
| Q16626 | J15ef3 | male-enhanced antigen | 1253.21 | 1530 | 1827.301 |
| Q9NQG1 | C06gh5 | mannosidase; beta A; lysosomal [symbol] | 533.2883 | 800 | 736.627 |
| P26572 | G11ab7 | mannosyl (alpha-1;3-)-glycoprote [symbol] | 619.6733 | 369 | 887.6535 |
| Q10469 | G15ab7 | mannosyl (alpha-1;6-)-glycoprote [symbol] | 600.1049 | 583 | 636.621 |
| P41223 | L09cd4 | maternal G10 transcript | 372.2443 | 563 | 575.7695 |
| Q9Y5V3 | G13cd6 | melanoma antigen: family D; 1 | 397.4495 | 952 | 1674.881 |
| P43121 | I22ef5 | melanoma cell adhesion molecu [symbol] | 886.0756 | 795 | 432.1718 |
| Q92494 | D11ef1 | membrane cofactor protein (CD4 | 973.362 | 1050 | 834.044 |
| Q14444 | H15ab7 | membrane component; chromos [symbol] | 549.4803 | 666 | 464.8174 |
| Q9NPE2 | O12ef2 | mesenchymal stem cell protein C | 749.8938 | 932 | 1085.26 |
| Q92571 | G07ab7 | mesoderm specific transcript hon | 617.581 | 446 | 537.1735 |
| O95204 | N11ef3 | metalloprotease 1 (pitrilysin fam [symbol] | 735.157 | 349 | 478.8153 |
| P80297 | H20ab7 | metallothionein 1L | 589.6327 | 502 | 656.4441 |
| Q99735 | C18ef7 | microsomal glutathione S-transfe [symbol] | 527.4698 | 686 | 373.9779 |
| Q98XW5 | D13gh8 | microtubule-associated protein 1 | 247.2817 | 692 | 887.6299 |
| Q9Y6C9 | M14ef3 | mitochondrial carrier homolog 2 | 1874.893 | 1910 | 1893.062 |
| Q9Y3B7 | E07ef2 | mitochondrial ribosomal protein 1 | 401.0368 | 421 | 732.113 |
| Q9P015 | A12ef8 | mitochondrial ribosomal protein 1 | 553.658 | 760 | 589.0617 |
| Q9NX20 | M06gh2 | mitochondrial ribosomal protein 1 | 642.9143 | 496 | 701.1442 |
| Q9H0U6 | O13ef8 | mitochondrial ribosomal protein 1 | 741.5383 | 1000 | 826.8816 |
| Q9NZE8 | O04ef2 | mitochondrial ribosomal protein 1 | 914.9722 | 910 | 956.7888 |
| Q9P0P3 | K18ef2 | mitochondrial ribosomal protein 1 | 1217.101 | 891 | 1196.143 |
| Q9Y317 | M15ef2 | mitochondrial ribosomal protein 1 | 799.6979 | 1020 | 1372.352 |
| Q9Y6G3 | C19ef8 | mitochondrial ribosomal protein 1 | 571.7264 | 810 | 689.3229 |
| Q9BYC7 | K12gh8 | mitochondrial ribosomal protein [symbol] | 490.5463 | 665 | 619.0928 |
| Q9Y3D3 | B12ef1 | mitochondrial ribosomal protein [symbol] | 966.7611 | 1020 | 1006.958 |
| Q9Y676 | C15ef8 | mitochondrial ribosomal protein [symbol] | 683.2889 | 580 | 761.9887 |
| Q9Y3D5 | G19ef2 | mitochondrial ribosomal protein [symbol] | 696.3306 | 1080 | 825.557 |
| P82921 | O11gh7 | mitochondrial ribosomal protein [symbol] | 748.082 | 1190 | 1115.5 |
| P82650 | D07gh4 | mitochondrial ribosomal protein [symbol] | 554.1784 | 515 | 470.683 |
| Q9Y2Q9 | A03ef8 | mitochondrial ribosomal protein [symbol] | 1439.076 | 1420 | 1329.749 |
| Q9NP92 | F02ef3 | mitochondrial ribosomal protein [symbol] | 669.1652 | 532 | 617.3223 |
| Q02750 | J08ef5 | mitogen-activated protein kinase | 615.689 | 701 | 453.8702 |
| Q02779 | K09ab7 | mitogen-activated protein kinase | 597.1743 | 423 | 509.6637 |
| Q12851 | N10ab8 | mitogen-activated protein kinase | 988.0971 | 1020 | 976.7391 |
| P49137 | J06ef5 | mitogen-activated protein kinase | 942.7459 | 1000 | 1068.579 |
| Q9BYG3 | O02gh8 | MKI67 (FHA domain) interacting | 791.8688 | 407 | 307.4404 |
| Q15014 | C17cd8 | MORF-related gene X | 1146.48 | 1030 | 1262.273 |
| | F22ef4 | mRNA; clone: PO2ST9 | 1052.442 | 1040 | 905.2614 |
| Q15773 | M18ef5 | myeloid leukemia factor 2 | 577.4585 | 467 | 766.6809 |
| P35579 | O01ef1 | myosin; heavy polypeptide 9; no [symbol] | 1426.003 | 1150 | 1203.964 |
| Q9Y6D2 | C21ef2 | N-acetyltransferase 5 (ARD1 ho [symbol] | 531.5485 | 708 | 274.9715 |
| P41227 | G13gh1 | N-acetyltransferase; homolog of | 800.6676 | 905 | 818.2554 |
| Q13510 | H20ab2 | N-acylsphingosine amidohydrola [symbol] | 752.1534 | 1660 | 677.5006 |
| O43678 | O09ab7 | NADH dehydrogenase (ubiquino [symbol] | 1366.754 | 1930 | 2137.906 |
| O95167 | E24ab7 | NADH dehydrogenase (ubiquino [symbol] | 1431.655 | 1770 | 1506.934 |
| P56556 | O11ab7 | NADH dehydrogenase (ubiquino [symbol] | 1650.612 | 916 | 795.8725 |
| O95182 | K06ab7 | NADH dehydrogenase (ubiquino [symbol] | 750.0893 | 777 | 725.1711 |
| P51970 | L04cd8 | NADH dehydrogenase (ubiquino [symbol] | 554.1995 | 728 | 1021.163 |
| O96000 | J10ab8 | NADH dehydrogenase (ubiquino [symbol] | 1110.197 | 1440 | 1111.615 |
| O95178 | J06ab8 | NADH dehydrogenase (ubiquino [symbol] | 925.3926 | 768 | 815.266 |
| O95139 | M03ab8 | NADH dehydrogenase (ubiquino [symbol] | 1392.873 | 1480 | 2162.279 |
| P17568 | H02ab8 | NADH dehydrogenase (ubiquino [symbol] | 787.0681 | 567 | 591.1924 |
| Q9Y6M9 | P16ab8 | NADH dehydrogenase (ubiquino [symbol] | 1299.803 | 1450 | 746.8017 |
| O43677 | M05ab8 | NADH dehydrogenase (ubiquino [symbol] | 586.5059 | 601 | 532.551 |
| O00217 | M09ab8 | NADH dehydrogenase (ubiquino [symbol] | 1383.411 | 1130 | 1194.001 |
| P19404 | M07ef1 | NADH dehydrogenase (ubiquino [symbol] | 1151.224 | 443 | 356.308 |
| O76008 | A12cd6 | Nef-associated factor 1 | 720.2744 | 951 | 424.04 |
| Q9UPY4 | K22ef3 | nesca protein | 425.9149 | 469 | 621.9336 |
| | L19gh8 | neurabin II | 1241.544 | 862 | 1023.077 |
| Q99742 | O07ab8 | neuronal PAS domain protein 1 | 1076.259 | 405 | 606.3589 |
| O60448 | P20ef3 | neuronal thread protein | 1831.592 | 1660 | 1490.624 |
| Q9UNW9 | O05ab8 | neuro-oncological ventral antiger | 1049.959 | 1050 | 1219.792 |
| Q9UGL9 | C09gh3 | NICE-1 protein | 1026.838 | 1360 | 381.058 |
| Q9UGL6 | M09ef4 | NICE-5 protein | 912.7542 | 1390 | 955.016 |
| Q92982 | I09ef7 | ninjurin 1 | 458.23 | 689 | 412.0181 |
| Q9BPW8 | N19cd3 | nipsnap homolog 1 (C. elegans) | 829.4266 | 529 | 562.9689 |
| Q9NQR4 | B13gh4 | Nit protein 2 | 519.1587 | 868 | 665.8632 |
| Q9UF10 | L08ef2 | non-canonical ubquitin conjugati [symbol] | 1336.406 | 2000 | 1488.008 |
| Q9Y385 | H11ef2 | non-canonical ubquitin conjugati [symbol] | 625.6356 | 808 | 669.812 |
| Q15233 | O02ef1 | non-POU domain containing; oct [symbol] | 1083.363 | 1210 | 1234.216 |
| Q13137 | A03cd6 | nuclear domain 10 protein | 910.7725 | 914 | 944.6079 |
| Q16236 | M03cd1 | nuclear factor (erythroid-derived | 1233.751 | 1180 | 1043.638 |
| P08651 | B07ef1 | nuclear factor I/C (CCAAT-bindin [symbol] | 726.0476 | 624 | 659.6305 |
| O95134 | P10cd3 | nuclear localization signal delete | 465.2933 | 522 | 536.7333 |
| Q15772 | N19ab2 | nuclear protein; marker for differ [symbol] | 1473.704 | 923 | 1157.202 |
| Q9UHY1 | L15cd8 | nuclear receptor binding protein | 515.949 | 948 | 619.7512 |
| Q9UPC9 | H23ab2 | nuclear receptor coactivator 3 | 822.7768 | 1190 | 932.9058 |
| Q15325 | N22cd2 | nuclease sensitive element bindi [symbol] | 1644.614 | 1720 | 2064.24 |
| O00567 | D05cd6 | nucleolar protein 5A (56 kD with [symbol] | 659.4335 | 608 | 623.2536 |
| Q9NX24 | A10gh3 | nucleolar protein family A; memb [symbol] | 466.2539 | 625 | 594.8443 |
| P06748 | K06gh6 | nucleophosmin (nucleolar phosp [symbol] | 1605.368 | 1080 | 1136.972 |
| P50583 | B01ab2 | nudix (nucleoside diphosphate li [symbol] | 1590.763 | 211 | 251.3768 |
| Q9UHM6 | N20gh8 | opsin 4 (melanopsin) | 1076.73 | 1200 | 1643.887 |
| Q9Y218 | P22gh1 | optineurin | 1014.697 | 1980 | 912.1917 |
| Q16612 | O01cd5 | P311 protein | 322.7444 | 349 | 2231.846 |
| P55771 | J21cd7 | paired box gene 9 | 692.506 | 380 | 573.135 |
| Q9UKJ1 | N21cd8 | paired immunoglobulin-like rece [symbol] | 674.1862 | 450 | 558.3225 |
| Q15165 | P24ab7 | paraoxonase 2 | 338.5244 | 891 | 583.7771 |
| Q07002 | P06ef5 | PCTAIRE protein kinase 3 | 836.5659 | 916 | 632.7298 |
| Q9Y3C6 | J07ef2 | peptidylprolyl isomerase (cyclop [symbol] | 548.5938 | 731 | 526.6097 |
| P30405 | F14cd5 | peptidylprolyl isomerase F (cyclo [symbol] | 843.1834 | 925 | 467.6738 |
| O43924 | G24ab8 | phosphodiesterase 6D; cGMP-s [symbol] | 373.1208 | 630 | 699.1887 |
| P17858 | K10ab8 | phosphofructokinase; liver | 950.5746 | 525 | 774.727 |
| Q01813 | C17ef1 | phosphofructokinase; platelet | 573.5211 | 646 | 555.9236 |
| O00511 | N20cd3 | phosphoprotein enriched in astrc | 1330.005 | 1500 | 2288.098 |
| P11216 | B16ab8 | phosphorylase; glycogen; brain | 516.7884 | 495 | 988.163 |
| P00749 | P19ef7 | plasminogen activator; urokinase | 1096.052 | 3440 | 1051.513 |
| P13797 | C13cd1 | plastin 3 (T isoform) | 990.1173 | 804 | 1393.037 |
| Q15102 | E16ab8 | platelet-activating factor acetylhy [symbol] | 469.9778 | 674 | 468.9765 |
| P01127 | A08ef5 | platelet-derived growth factor bel [symbol] | 500.6466 | 674 | 456.4576 |
| Q15795 | C19ef6 | pleckstrin homology; Sec7 and [symbol] | 578.295 | 740 | 518.8327 |
| O43660 | O18ab8 | pleiotropic regulator 1 (PRL 1hor [symbol] | 743.7263 | 933 | 804.3456 |
| Q15155 | G12ef3 | pM5 protein | 664.2511 | 423 | 490.295 |
| Q9H361 | L04gh7 | poly(A) binding protein; cytoplas [symbol] | 759.4566 | 491 | 710.9543 |
| Q15366 | A11cd1 | poly(rC) binding protein 2 | 1951.241 | 1490 | 2128.585 |
| P19388 | B15ab8 | polymerase (RNA) II (DNA direct | 552.3545 | 529 | 698.4965 |
| P52434 | F05gh6 | polymerase (RNA) II (DNA direct | 949.7064 | 1480 | 1210.411 |
| P36954 | A20cd1 | polymerase (RNA) II (DNA direct | 1603.557 | 1820 | 1413.573 |
| P53803 | C17cd1 | polymerase (RNA) II (DNA direct | 437.9544 | 893 | 528.0144 |
| Q9UQQ3 | J08cd6 | POP4 (processing of precursor; | 480.5848 | 645 | 551.3661 |
| Q13670 | B07ab8 | postmeiotic segregation increase | 812.7865 | 779 | 532.0548 |
| Q9P0J7 | L07gh4 | potassium channel modulatory fa [symbol] | 1401.591 | 1720 | 1192.204 |
| O60925 | K02ab8 | prefoldin 1 | 1631.996 | 1720 | 1860.632 |
| Q9UHV9 | J15ef4 | prefoldin 2 | 514.4897 | 882 | 833.1525 |
| Q13519 | J07ef6 | prepronociceptin | 564.2079 | 464 | 887.4522 |
| P04156 | A13ab8 | prion protein (p27-30) Creutzfel [symbol] | 797.9495 | 631 | 701.8217 |
| Q9UI73 | K03ef8 | PRO0246 protein | 430.412 | 535 | 546.8599 |
| Q9P0T3 | D14ef1 | proapoptotic caspase adaptor p [symbol] | 1261.463 | 1650 | 2553.652 |
| Q02809 | P20ab7 | procollagen-lysine; 2-oxoglutarat | 503.7557 | 477 | 1993.232 |
| P07237 | G13ab8 | procollagen-proline; 2-oxoglutar [symbol] | 633.8309 | 406 | 575.6635 |
| P09466 | E12ab8 | progestagen-associated endome | 367.0939 | 625 | 708.8717 |
| Q16342 | G22ef6 | programmed cell death 2 | 735.0961 | 759 | 490.3938 |
| O75340 | I02cd8 | programmed cell death 6 | 1055.886 | 1090 | 1358.597 |
| P35232 | C22ef5 | porhibitin | 2045.693 | 1810 | 1600.236 |
| Q9UQ80 | O03cd1 | proliferation-associated 2G4; 38 [symbol] | 1902.387 | 1390 | 2003.135 |
| Q12796 | E05ab3 | proline rich 2 | 1927.78 | 1530 | 1475.973 |
| Q15188 | P20cd2 | proline-rich protein BstNI subfa [symbol] | 997.5582 | 1290 | 1404.4 |
| P35998 | L09ab8 | proteasome (prosome; macropali [symbol] | 1010.463 | 1200 | 1059.594 |
| P47210 | N16cd2 | proteasome (prosome; macropali [symbol] | 1037.339 | 1150 | 1193 |
| O00495 | N03ab8 | proteasome (prosome; macropali [symbol] | 1043.875 | 498 | 563.7775 |
| O75831 | N07ab8 | proteasome (prosome; macropali [symbol] | 763.7632 | 622 | 506.8297 |
| Q13200 | L15ab8 | proteasome (prosome; macropali [symbol] | 644.3008 | 594 | 331.4288 |
| O43242 | L17ab8 | proteasome (prosome; macropali [symbol] | 2159.327 | 1230 | 1637.67 |
| P55036 | N18cd2 | proteasome (prosome; macropali [symbol] | 460.8093 | 724 | 588.5346 |
| P48556 | L21ab8 | proteasome (prosome; macropali [symbol] | 1240.243 | 1030 | 1685.116 |
| Q92530 | G07cd6 | proteasome (prosome; macropali [symbol] | 1804.777 | 1570 | 1800.731 |
| P28066 | J13ab8 | proteasome (prosome; macropali [symbol] | 766.589 | 767 | 775.2704 |
| O14818 | J15ab8 | proteasome (prosome; macropali [symbol] | 1242.517 | 1540 | 1139.994 |
| P49721 | J19ab8 | proteasome (prosome; macropali [symbol] | 1832.921 | 2110 | 1617.062 |
| P28062 | H18ab8 | proteasome (prosome; macropali [symbol] | 797.3912 | 905 | 505.3638 |
| P28065 | B06ef7 | proteasome (prosome; macropali [symbol] | 771.1998 | 1630 | 1002.72 |
| P10619 | A07ab8 | protective protein for beta-galac [symbol] | 1539.157 | 907 | 980.3046 |
| Q9UNN8 | F03cd6 | protein C receptor; endothelial (E [symbol] | 892.1589 | 702 | 488.5084 |
| Q15084 | H02cd5 | protein disulfide isomerase-relat [symbol] | 1081.309 | 407 | 859.0228 |
| Q9Y2B9 | C22cd7 | protein kinase (cAMP-dependent | 650.9588 | 1040 | 754.6101 |
| Q13517 | A09ef5 | protein kinase C binding protein | 380.9448 | 587 | 630.5497 |
| P54619 | F15ab8 | protein kinase; AMP-activated; g [symbol] | 482.8359 | 572 | 841.1746 |
| P17612 | F13ab8 | protein kinase; cAMP-dependent | 643.0385 | 629 | 901.5293 |
| P09131 | F10gh1 | Protein P3 | 529.0069 | 607 | 617.0621 |
| O60927 | P02gh1 | protein phosphatase 1; regulator | 1728.646 | 1760 | 1404.955 |
| P41236 | C08cd1 | protein phosphatase 1; regulator | 532.1136 | 621 | 583.7663 |
| Q15435 | D15ab8 | protein phosphatase 1; regulator | 634.9759 | 963 | 753.9046 |
| P05323 | E13ef6 | protein phosphatase 2 (formerly [symbol] | 1058.276 | 1120 | 796.6526 |
| Q15257 | G01ef5 | protein phosphatase 2A; regulat [symbol] | 925.4458 | 905 | 1160.437 |
| P33172 | F01ab8 | protein phosphatase 4 (formerly [symbol] | 1791.171 | 1220 | 1064.665 |
| O00743 | F03ab8 | protein phosphatase 6; catalytic [symbol] | 998.7 | 1070 | 738.4815 |
| P38391 | E21cd7 | protein translocation complex be [symbol] | 1049.306 | 1050 | 1428.451 |
| P78324 | M17cd4 | protein tyrosine phosphatase; no | 639.2381 | 741 | 591.4877 |
| Q13332 | P21ab8 | protein tyrosine phosphatase; re [symbol] | 1882.284 | 1880 | 2019.865 |
| Q9HAD6 | C08gh6 | protocadherin 16 dachsous-like ( | 967.2552 | 906 | 1070.494 |
| O43445 | E11cd5 | PRP4 pre-mRNA processing fact [symbol] | 777.1081 | 549 | 569.3749 |
| Q9Y6B3 | L19ef2 | PTD013 protein | 652.1251 | 701 | 1031.739 |
| Q9HAN1 | P19gh6 | pumillo homolog 1 (Drosophila) | 842.4925 | 1170 | 1047.18 |
| Q9NQA4 | C23ef4 | putative acid-sensing ion channe [symbol] | 742.723 | 762 | 678.3813 |
| O43598 | J05cd6 | putative c-Myc-responsive | 473.6498 | 621 | 459.383 |
| O43257 | M12cd6 | putative cyclin G1 interacting pro [symbol] | 1118.374 | 1190 | 1238.925 |
| Q9NY06 | M07ef4 | putative integral membrane trans [symbol] | 966.4384 | 544 | 569.8565 |
| P17152 | H21cd4 | putative receptor protein | 399.9305 | 650 | 591.3162 |
| Q9Y6I9 | B13ef2 | putative secreted protein | 373.307 | 485 | 742.537 |
| P54886 | B12ab8 | pyrroline-5-carboxylate syntheta [symbol] | 406.7128 | 302 | 905.8993 |
| Q13876 | B03ef7 | quiescin Q6 | 745.299 | 585 | 842.2489 |
| O14679 | K24ef5 | quinone oxidoreductase homolo [symbol] | 710.5753 | 1160 | 651.2257 |
| P53611 | N16ab8 | Rab geranylgeranyltransferase; [symbol] | 758.1496 | 727 | 938.6179 |
| P47224 | D06ab8 | RAB interacting factor | 645.5902 | 859 | 577.3956 |
| Q15907 | H04cd4 | RAB11B; member RAS oncogen [symbol] | 926.8757 | 1030 | 1224.758 |
| P51153 | D04ab8 | RAB13; member RAS oncogene | 619.0867 | 935 | 1131.86 |
| P35287 | J24ef2 | RAB14; member RAS oncogene | 543.7425 | 584 | 586.1635 |
| Q9NP72 | P02ef8 | RAB18; member RAS oncogene | 459.4786 | 794 | 634.6616 |
| P11476 | H22ab6 | RAB1A; member RAS oncogene | 1039.455 | 767 | 993.586 |
| Q9H0U4 | L08gh7 | RAB1B; member RAS oncogene | 526.9882 | 691 | 696.5292 |
| P08886 | B22ab8 | RAB2; member RAS oncogene f [symbol] | 1483.703 | 1900 | 2195.304 |
| O95716 | P04cd4 | RAB3D; member RAS oncogene | 908.9015 | 638 | 556.2242 |
| P35239 | D02ab8 | RAB5B; member RAS oncogene | 598.5113 | 708 | 393.3892 |
| P20340 | C07ef6 | RAB6A; member RAS oncogene | 503.1507 | 655 | 416.7552 |
| P43487 | D16ab8 | RAN binding protein 1 | 1438.305 | 1180 | 1786.177 |
| Q9UI26 | B20ef1 | Ran binding protein 11 | 1505.224 | 1180 | 1273.677 |
| P47736 | D18ab8 | RAP1; GTPase activating protei [symbol] | 875.8692 | 414 | 461.3322 |
| Q15382 | F17cd2 | Ras homolog enriched in brain 2 | 1435.587 | 2190 | 1635.792 |
| P52199 | B18ab2 | ras homolog gene family; memb [symbol] | 534.6169 | 1520 | 399.0517 |
| P35238 | B20ab2 | ras homolog gene family; memb [symbol] | 1022.646 | 872 | 1019.472 |
| Q14644 | J08ef7 | RAS p21 protein activator (GTPa | 615.946 | 623 | 627.059 |
| Q15404 | A10cd8 | Ras suppressor protein 1 | 1543.953 | 1920 | 2317.155 |
| Q13283 | H14cd5 | Ras-GTPase-activating protein S | 846.7267 | 789 | 571.1476 |
| Q9NVQ9 | M05gh1 | Rec8p; a meiotic recombination [symbol] | 1334.371 | 1200 | 1325.85 |
| O60895 | K17cd6 | receptor (calcitonin) activity modi [symbol] | 572.8174 | 519 | 899.2606 |
| P35244 | I22ef6 | replication protein A3 (14 kD) | 544.0212 | 502 | 924.5385 |
| Q99623 | D03cd7 | repressor of estrogen receptor a [symbol] | 1139.283 | 689 | 1233.151 |
| Q9UMQ4 | C01gh1 | ret proto-oncogene (multiple end | 769.2484 | 602 | 803.931 |
| Q9NQC3 | M19cd7 | reticulon 4 | 1459.586 | 586 | 927.9776 |
| P47804 | P03cd1 | retinal G protein coupled recepto [symbol] | 403.1219 | 497 | 627.4176 |
| Q16576 | P07ef6 | retinoblastoma binding protein 7 | 1539.006 | 1410 | 798.8744 |
| O95357 | D02cd4 | retinoic acid induced 3 | 678.1926 | 713 | 206.6352 |
| Q15299 | D20ef1 | retinoic acid receptor-beta assoc [symbol] | 1060.232 | 979 | 1013.444 |
| Q9UMY1 | P05ef2 | retinoic acid repressible protein | 610.5279 | 726 | 586.2413 |
| Q13017 | M15ef6 | Rho GTPase activating protein 5 | 560.443 | 807 | 514.6138 |
| O43182 | B24ab2 | Rho GTPase activating protein 6 | 1257.146 | 806 | 895.7191 |
| O60274 | F04gh6 | Rho-specific guanine nucleotide | 651.9484 | 577 | 366.4761 |
| P07998 | P21cd1 | ribonuclease; RNase A family; 1 | 1294.476 | 3040 | 1592.811 |
| P04843 | B12cd1 | ribophorin I | 1493.814 | 1360 | 2248.214 |
| P04844 | B14cd1 | ribophorin II | 1574.181 | 1170 | 1880.047 |
| P26373 | D11cd1 | ribosomal protein L13 | 2091.256 | 1200 | 1924.546 |
| P40429 | H12ef1 | ribosomal protein L13a | 1928.024 | 2060 | 1091.46 |
| P39019 | J05cd1 | ribosomal protein S19 | 1794.644 | 1480 | 2253.771 |
| P78317 | B04cd1 | ring finger protein 4 | 756.2395 | 661 | 668.8027 |
| Q99942 | F04cd2 | ring finger protein 5 | 1016.837 | 949 | 1126.187 |
| O43148 | A15cd4 | RNA (guanine-7-) methyltransfer [symbol] | 572.6495 | 730 | 390.498 |
| O02916 | F06ab8 | RNA binding motif protein 4 | 533.7121 | 628 | 430.5298 |
| Q9Y580 | J10gh6 | RNA binding motif protein 7 | 368.8423 | 710 | 558.043 |
| Q14621 | H07cd7 | RNA binding protein (autoantige [symbol] | 499.5699 | 737 | 506.822 |
| Q93062 | K17cd7 | RNA-binding protein gene with r [symbol] | 845.0994 | 659 | 640.4514 |
| Q99497 | B15cd7 | RNA-binding protein regulatory s [symbol] | 1914.86 | 1400 | 2563.974 |
| Q99584 | L17cd2 | S100 calcium binding protein A1 [symbol] | 492.6196 | 421 | 675.1457 |
| P23526 | A06ef1 | S-adenosylhomocysteine hydrol [symbol] | 550.6181 | 603 | 478.692 |
| O43865 | H21ab2 | S-adenosylhomocysteine hydrol [symbol] | 1460.581 | 526 | 466.4887 |
| O14826 | D02cd5 | secretory carrier membrane prot [symbol] | 661.3975 | 460 | 481.9262 |
| O75326 | L05cd3 | sema domain; immunoglobulin d [symbol] | 795.0564 | 768 | 943.1132 |
| O43278 | I16cd2 | serine protease inhibitor; Kunitz [symbol] | 1295.263 | 1260 | 641.0775 |
| O00271 | C05ef5 | serine protease inhibitor; Kunitz [symbol] | 1379.545 | 1740 | 876.4128 |
| Q9UEW8 | N22ef5 | serine threonine kinase 39 (STE [symbol] | 1230.117 | 1030 | 841.1872 |
| Q13043 | P15cd2 | serine/threonine kinase 4 | 750.7132 | 838 | 756.1662 |
| Q9Y282 | F01ef2 | serologically defined breast canc [symbol] | 1279.414 | 1160 | 1907.544 |
| P35542 | A02gh1 | serum amyloid A4; constitutive | 754.5041 | 1310 | 811.5701 |
| O75790 | I22cd6 | seven transmembrane domain pr [symbol] | 761.3872 | 778 | 799.0998 |
| Q9P0V3 | H17ef3 | SH3-domain binding protein 4 | 1204.005 | 1710 | 1539.363 |
| Q99519 | C13ab8 | sialidase 1 (lysosomal sialidase) | 712.4593 | 680 | 498.7449 |
| Q92185 | J20cd1 | sialytransferase 8A (alpha-N-ac [symbol] | 622.4236 | 478 | 462.7941 |
| Q9Y6A9 | C05ef8 | signal peptidase 12 kDa | 449.125 | 742 | 887.4615 |
| P21378 | I04ef3 | signal peptidase complex (18 kD) | 630.5617 | 515 | 732.1592 |
| O15302 | P10ab3 | signal recognition particle 72 kD | 1040.23 | 818 | 841.5078 |
| O76094 | J02cd2 | signal recognition particle 72 kD | 1473.56 | 965 | 1422.2 |
| Q9UNL2 | J10cd2 | signal sequence receptor; gamm [symbol] | 1150.724 | 450 | 1654.744 |
| P40763 | K09ef5 | signal transducer and activator o [symbol] | 671.5038 | 545 | 348.0347 |
| Q92783 | M24cd3 | signal transducing adaptor mole [symbol] | 827.9851 | 1020 | 725.7017 |
| Q9H723 | N01gh5 | similar to rat nuclear ubiquitous [symbol] | 749.7661 | 530 | 1258.65 |
| O15258 | A04cd7 | similar to S. cerevisiae RER1 | 710.8499 | 635 | 933.6382 |
| O14834 | A11cd7 | similar to S. pombe dim1+ | 701.2292 | 753 | 903.8926 |
| O43617 | H05ef3 | similar to yeast BET3 (S. cerevis [symbol] | 1759.348 | 1750 | 1443.578 |
| Q04837 | G11cd2 | single-stranded DNA binding pro [symbol] | 1264.277 | 894 | 1034.75 |
| P05455 | E19gh1 | Sjogren syndrome antigen B (aut [symbol] | 850.9691 | 744 | 930.6428 |
| P43331 | K08cd2 | small nuclear ribonucleoprotein [symbol] | 916.3597 | 1260 | 1042.335 |
| P09012 | M12cd2 | small nuclear ribonucleoprotein [symbol] | 548.0261 | 310 | 685.6636 |
| P08579 | A03cd2 | small nuclear ribonucleoprotein [symbol] | 502.6355 | 570 | 669.9879 |
| P09234 | A05cd2 | small nuclear ribonucleoprotein [symbol] | 1054.327 | 1450 | 1302.742 |
| P08578 | A07cd2 | small nuclear ribonucleoprotein [symbol] | 1901.214 | 1740 | 1818.557 |
| Q15357 | A11cd2 | small nuclear ribonucleoprotein [symbol] | 382.4892 | 734 | 593.847 |
| P14678 | C23cd3 | small nuclear ribonucleoprotein [symbol] | 2036.574 | 1280 | 1737.398 |
| Q13487 | P08cd1 | small nuclear RNA activating co [symbol] | 517.5819 | 684 | 648.0121 |
| P35326 | E17cd3 | small proline-rich protein 2A | 290.6174 | 1030 | 839.3072 |
| P55854 | H04cd2 | SMT3 suppressor of mif two 3 ho [symbol] | 791.7437 | 658 | 962.4453 |
| P14648 | P19cd5 | SNRPN upstream reading frame | 1581.907 | 2270 | 1670.964 |
| P55011 | L17cd1 | solute carrier family 12 (sodium [symbol] | 591.8676 | 740 | 517.2382 |
| O95258 | P15cd4 | solute carrier family 25 (mitochor [symbol] | 476.3075 | 518 | 577.8319 |
| O15431 | A21ab5 | solute carrier family 31 (copper t [symbol] | 1227.872 | 1010 | 1769.026 |
| P04920 | L04cd1 | solute carrier family 4; anion exc [symbol] | 1614.362 | 1560 | 1704.134 |
| Q01650 | J11ef5 | solute carrier family 7 (cationic a [symbol] | 832.6996 | 632 | 1032.106 |
| Q07890 | H23cd2 | son of sevenless homolog 2 (Dro [symbol] | 476.9505 | 568 | 680.8529 |
| O60749 | A19cd2 | sorting nexin 2 | 364.9343 | 931 | 769.5782 |
| O60493 | A09cd4 | sorting nexin 3 | 1008.916 | 1350 | 1180.469 |
| Q9Y5X3 | G06ef4 | sorting nexin 5 | 676.0975 | 647 | 688.5568 |
| Q01082 | E09cd2 | spectrin; beta; non-erythrocytic 1 | 571.4909 | 679 | 387.0403 |
| P52788 | M10cd2 | spermine synthase | 895.1994 | 854 | 770.002 |
| P17947 | C19cd2 | spleen focus forming virus (SFF [symbol] | 1307.418 | 1150 | 1646.278 |
| Q14818 | D10ef6 | splicing factor 1 | 547.0998 | 499 | 782.2929 |
| Q15427 | K11cd6 | splicing factor 3b; subunit 4; 49k [symbol] | 441.2394 | 612 | 540.0723 |
| Q15815 | M06cd2 | splicing factor; arginine/serine-ri [symbol] | 738.2591 | 553 | 551.9082 |
| Q01130 | H22cd1 | splicing factor; arginine/serine-ri [symbol] | 553.5808 | 527 | 488.5465 |
| P23152 | H24cd1 | splicing factor; arginine/serine-ri [symbol] | 1547.474 | 1460 | 1426.273 |
| Q13503 | L22cd4 | SRB7 suppressor of RNA polym [symbol] | 712.8859 | 574 | 725.0591 |
| Q9P2R9 | C23ef2 | SRp25 nuclear protein | 495.796 | 558 | 515.095 |
| Q9H6I2 | F07gh5 | SRY (sex determining region Y)- [symbol] | 774.5162 | 970 | 714.8666 |
| Q9BT81 | I13gh8 | SRY (sex determining region Y)- [symbol] | 497.0608 | 530 | 550.1706 |
| O60526 | M03cd6 | STIP1 homology and U-Box cont [symbol] | 706.6078 | 538 | 543.7559 |
| Q9Y6X1 | A21ef4 | stress-associated endoplasmic [symbol] | 1683.36 | 1220 | 2201.647 |
| P31948 | M15ef3 | stress-induced-phosphoprotein 1 | 436.1143 | 549 | 517.5834 |
| P21912 | E09gh1 | succinate dehydrogenase compl [symbol] | 701.7228 | 902 | 751.7357 |
| O14521 | H04cd1 | succinate dehydrogenase compl [symbol] | 628.8405 | 505 | 672.375 |
| P53597 | I09gh1 | succinate-CoA ligase; GDP-form [symbol] | 704.4006 | 491 | 553.293 |
| O95605 | P15cd5 | SUMO-1 activating enzyme subu [symbol] | 776.4199 | 499 | 433.2742 |
| Q16550 | K01cd2 | suppressor of Ty 4 homolog 1 (S [symbol] | 813.5735 | 1440 | 991.3588 |
| Q15526 | K07cd2 | surfeit 1 | 468.3614 | 522 | 583.8097 |
| O43539 | P02cd1 | SWI/SNF related; matrix associa [symbol] | 1047.14 | 1280 | 1212.614 |
| Q9UPX1 | D23cd7 | synaptopodin | 683.4973 | 536 | 421.1841 |
| O95721 | O09cd5 | synaptosomal-associated protein | 830.7721 | 1030 | 686.6755 |
| O43391 | H09cd2 | syndecan binding protein (synter | 625.6962 | 640 | 379.1899 |
| Q15833 | J04cd2 | syntaxin binding protein 2 | 661.0175 | 556 | 840.6951 |
| Q92804 | O20cd3 | TAF15 RNA polymerase II; TAT [symbol] | 873.5584 | 605 | 1063.778 |
| Q9Y490 | B04cd2 | talin 1 | 1342.238 | 1170 | 2365.625 |
| O94797 | H20ef7 | Tara-like protein | 899.0807 | 448 | 496.1404 |
| Q13311 | C05cd6 | Tax1 (human T-cell leukemia vir [symbol] | 899.9979 | 1190 | 926.2314 |
| Q16650 | F08cd6 | T-box; brain; 1 | 1407.309 | 1750 | 1777.889 |
| P51864 | O04ef7 | teratocarcinoma-derived growth [symbol] | 2037.212 | 1590 | 1447.46 |
| P55061 | O19cd2 | testis enhanced gene transcript ( | 1540.968 | 1330 | 1972.323 |
| Q15569 | P21cd2 | testis-specific kinase 1 | 837.6269 | 796 | 708.1178 |
| O60636 | F08cd5 | tetraspan 2 | 468.1664 | 702 | 375.3225 |
| O60637 | F06cd5 | tetraspan 3 | 1025.362 | 742 | 545.9662 |
| O95857 | F13ef3 | tetraspan NET-6 protein | 702.1579 | 676 | 359.1921 |
| Q99614 | M03cd3 | tetratricopeptide repeat domain 1 | 706.3536 | 891 | 635.5379 |
| Q13118 | L05cd2 | TGFB inducible early growth res [symbol] | 445.9691 | 601 | 679.2931 |
| Q15583 | A16cd2 | TGFB-induced factor (TALE fami [symbol] | 885.9 | 1290 | 1140.439 |
| Q9H3N1 | N03gh7 | thioredoxin domain-containing | 409.6333 | 426 | 686.7997 |
| O43396 | O17cd5 | thioredoxin-like; 32 kD | 1821.595 | 1710 | 1548.596 |
| P04216 | B02cd2 | Thy-1 cell surface antigen | 169.8064 | 298 | 4157.367 |
| P12956 | K10ef6 | thyroid autoantigen 70 kD (Ku an [symbol] | 1172.56 | 1490 | 1395.803 |
| P16035 | P11ef7 | tissue inhibitor of metalloprotein [symbol] | 360.2533 | 354 | 913.1351 |
| Q9H2X8 | I08gh8 | TLH29 protein precursor | 710.2447 | 1000 | 1386.102 |
| Q13077 | L11cd2 | TNF receptor-associated factor 1 | 1265.054 | 1270 | 756.401 |
| Q9NZ34 | P08gh4 | TPA regulated locus | 1074.319 | 1090 | 1284.44 |
| Q15369 | D18ef6 | transcription elongation factor B [symbol] | 771.5598 | 1100 | 693.1767 |
| Q15906 | P11ef6 | transcription factor-like 1 | 436.5561 | 510 | 878.9083 |
| P50616 | H08cd5 | transducer of ERBB2; 1 | 888.8059 | 874 | 518.1816 |
| Q13595 | N04ef7 | transformer-2 alpha (htra-2 alpha | 247.4437 | 612 | 698.379 |
| P37802 | F05cd3 | transgelin 2 | 1986.716 | 2240 | 1173.434 |
| Q9Y5Z9 | D01cd8 | transitional epithelia response pr [symbol] | 1324.284 | 875 | 1126.793 |
| Q99595 | K10cd6 | translocase of inner mitochondri [symbol] | 450.6578 | 665 | 448.5161 |
| O60830 | A09cd6 | translocase of inner mitochondri [symbol] | 1646.564 | 1590 | 1992.925 |
| O14925 | I24cd6 | translocase of inner mitochondri [symbol] | 745.8102 | 813 | 653.4301 |
| Q9Y5J9 | E08cd8 | translocase of inner mitochondri [symbol] | 1641.377 | 1550 | 1653.524 |
| Q15629 | G18ef3 | translocating chain-associating n [symbol] | 1003.031 | 1060 | 624.4499 |
| Q99442 | C10cd2 | translocation protein 1 | 526.8783 | 772 | 1065.397 |
| Q99805 | A18cd5 | transmembrane 9 superfamily m [symbol] | 1023.368 | 1260 | 957.2801 |
| Q9Y2B0 | C20ef3 | transmembrane protein 4 | 681.3857 | 1320 | 985.8397 |
| P49755 | J05ef4 | transmembrane trafficking protei [symbol] | 1311.203 | 469 | 700.7243 |
| | F03gh4 | transmembrane; prostate androg | 1287.98 | 841 | 662.4165 |
| Q03519 | B07cd1 | transporter 2; ATP-binding casse [symbol] | 851.2148 | 573 | 680.8519 |
| Q9NWF9 | P02gh2 | TRIAD3 protein | 665.9772 | 444 | 688.6646 |
| Q15656 | O07ef7 | TRK-fused gene | 1878.254 | 1640 | 1858.307 |
| Q92734 | J17cd7 | TRK-fused gene | 1450.307 | 1810 | 1609.067 |
| Q12815 | A21ef7 | trophinin associated protein (last | 821.5601 | 1100 | 722.8644 |
| P09493 | O02cd1 | tropomyosin 1 (alpha) | 988.1694 | 429 | 1004.982 |
| P49411 | M11cd3 | Tu translation elongation factor; [symbol] | 525.8572 | 555 | 476.4592 |
| P05217 | F03gh6 | tubulin; beta; 2 | 1203.686 | 705 | 920.1831 |
| P23258 | K03cd3 | tubulin; gamma 1 | 1733.971 | 1220 | 2149.365 |
| Q9UBN6 | A16ef6 | tumor necrosis factor receptor su [symbol] | 470.7412 | 751 | 531.524 |
| Q13829 | L22ef4 | tumor necrosis factor; alpha-indu [symbol] | 849.276 | 792 | 552.7776 |
| O00496 | K21cd3 | tumor suppressing subtransferab [symbol] | 628.3509 | 554 | 800.8751 |
| Q99816 | G04cd4 | tumor susceptibility gene 101 | 723.8133 | 766 | 627.1295 |
| P16422 | C03ab7 | tumor-associated calcium signal | 1448.512 | 791 | 373.663 |
| Q9NP84 | K03ef2 | type I transmembrane protein Fn | 691.89 | 498 | 560.2824 |
| Q9UJ47 | O09cd8 | type I transmembrane receptor ( [symbol] | 792.9873 | 819 | 809.9056 |
| P31946 | K21ef6 | tyrosine 3-monooxygenase/tryp [symbol] | 1220.989 | 955 | 724.9898 |
| P35214 | J19ef4 | tyrosine 3-monooxygenase/tryp [symbol] | 881.0131 | 989 | 682.5327 |
| P27348 | G15cd7 | tyrosine 3-monooxygenase/tryp [symbol] | 2196.334 | 1540 | 1104.855 |
| O60704 | J01cd3 | tyrosylprotein sulfotransferase 2 | 658.3761 | 299 | 686.1339 |
| Q9Y4Z0 | G09cd8 | U6 snRNA-associated Sm-like pr [symbol] | 837.5087 | 689 | 914.9547 |
| P14927 | G06cd4 | ubiquinol-cytochrome c reductas [symbol] | 995.1237 | 817 | 954.3085 |
| Q9UI02 | P18ef7 | ubiquinol-cytochrome c reductas [symbol] | 953.6884 | 1460 | 1546.568 |
| P31930 | A22cd3 | ubiquinol-cytochrome c reductas [symbol] | 846.6396 | 849 | 686.9864 |
| P22695 | A24cd3 | ubiquinol-cytochrome c reductas [symbol] | 468.5936 | 636 | 581.791 |
| P07919 | E08cd4 | ubiquinol-cytochrome c reductas [symbol] | 1328.532 | 1810 | 1005.378 |
| P47985 | E06cd4 | ubiquinol-cytochrome c reductas [symbol] | 1519.763 | 1270 | 1127.575 |
| Q9NZ09 | N17ef1 | ubiquitin associated protein | 1588.177 | 1820 | 1392.592 |
| Q9UNP0 | F09gh6 | ubiquitin specific protease 15 | 525.3364 | 492 | 597.7817 |
| Q9UHP3 | L21cd8 | ubiquitin specific protease 25 | 554.0657 | 724 | 561.5817 |
| P22314 | O07cd3 | ubiquitin-activating enzyme E1 ( [symbol] | 1857.238 | 1640 | 1863.316 |
| Q13404 | O23cd3 | ubiquitin-conjugating enzyme E2 | 1073.414 | 1210 | 1128.962 |
| P51669 | D05cd4 | ubiquitin-conjugating enzyme E2 | 1266.25 | 1300 | 1173.73 |
| P47986 | O11cd3 | ubiquitin-conjugating enzyme E2 | 616.3689 | 520 | 567.7317 |
| O14933 | H14cd4 | ubiquitin-conjugating enzyme E2 | 717.4244 | 893 | 596.6415 |
| Q16781 | D07cd4 | ubiquitin-conjugating enzyme E2 | 923.6537 | 816 | 533.7389 |
| Q93068 | A02cd3 | ubiquitin-like 1 (sentrin) | 1376.087 | 1530 | 1294.307 |
| Q15386 | I10gh1 | ubiquitin-protein isopeptide ligas [symbol] | 531.8659 | 630 | 628.2454 |
| Q9UBK9 | I15cd4 | ubiquitously-expressed transcrip [symbol] | 936.2619 | 1060 | 1384.764 |
| O60512 | E11ab3 | UDP-Gal: betaGlcNAc beta 1; 4- [symbol] | 436.7634 | 610 | 542.39 |
| P78383 | P20cd5 | UDP-galactose transporter relat [symbol] | 1570.135 | 1700 | 2180.117 |
| Q16851 | I24cd4 | UDP-glucose pyrophosphorylase | 1324.08 | 1260 | 1318.78 |
| Q9NZ45 | L02gh3 | uncharacterized hematopoietic s [symbol] | 652.0685 | 670 | 696.5692 |
| Q9NZ32 | L20gh4 | uncharacterized hypothalamus p [symbol] | 1035.575 | 1250 | 1354.231 |
| Q9NZ29 | L16gh4 | uncharacterized hypothalamus p [symbol] | 659.53 | 992 | 1118.776 |
| Q15853 | C02cd3 | upstream transcription factor 2; c [symbol] | 601.6839 | 635 | 698.0128 |
| Q92528 | D02ef7 | uridine monophosphate kinase | 560.3976 | 685 | 623.5243 |
| P15692 | M04ef7 | vascular endothelial growth facto [symbol] | 460.9257 | 539 | 857.3853 |
| P50552 | C08cd3 | vasodilator-stimulated phosphop [symbol] | 468.4573 | 648 | 392.5569 |
| O60763 | H16cd3 | vesicle docking protein p115 | 639.538 | 543 | 745.4806 |
| Q15836 | A15ef5 | vesicle-associated membrane p [symbol] | 804.2648 | 1370 | 1060.211 |
| Q9UEU0 | O22cd6 | vesicle-associated soluble NSF [symbol] | 468.6024 | 697 | 698.3534 |
| P14921 | E24ef5 | v-ets erythroblastosis virus E26 [symbol] | 913.893 | 765 | 291.1538 |
| P18206 | C14cd3 | vinculin | 580.3315 | 857 | 865.7505 |
| P00540 | G08ef5 | v-mos Moloney murine sarcoma [symbol] | 1115.145 | 728 | 663.6747 |
| P01106 | E18ef5 | v-myc myelocytomatosis viral on [symbol] | 537.8131 | 601 | 405.1047 |
| P21796 | C16cd3 | voltage-dependent anion channe [symbol] | 483.8969 | 510 | 584.201 |
| Q15765 | C12cd3 | von Hippel-Lindau binding prote [symbol] | 689.743 | 705 | 891.2621 |
| P10398 | D01ab4 | v-raf murine sarcoma 3611 viral [symbol] | 1061.812 | 639 | 532.2153 |
| P11233 | A19ef6 | v-ral simian leukemia viral oncog | 848.7099 | 1060 | 1081.428 |
| Q9Y6W5 | A22gh1 | WAS protein family; member 2 | 1283.077 | 1630 | 1493.145 |
| Q9UPY6 | L06cd6 | WAS protein family; member 3 | 1503.584 | 1210 | 2151.643 |
| O75083 | D15cd5 | WD repeat domain 1 | 892.4425 | 977 | 963.3448 |
| O76071 | A24cd5 | WD40 protein Clao1 | 609.8821 | 898 | 730.5311 |
| Q9HCN4 | H11gh6 | XPA binding protein 1; putative [symbol] | 1199.21 | 1070 | 507.3277 |
| Q9UM05 | N17ef3 | yeast Sec31p homolog | 1887.447 | 1590 | 1542.778 |
| Q9UQR1 | P06gh1 | zinc finger protein 148 (pHZ-52) | 513.8639 | 737 | 563.7625 |
| Q14119 | G21ef5 | zinc finger protein 161 | 1199.558 | 1760 | 1197.34 |
| Q9NW07 | F06gh2 | zinc finger protein 358 | 402.8342 | 575 | 684.7069 |
| P20694 | D04ef6 | zinc finger protein 9 (a cellular re [symbol] | 1042.65 | 1010 | 856.5924 |

| SwissProt | allMean | ratioItoA | rationIntto [symbol] | classID | classsName |
|---|---|---|---|---|---|
| P04270 | 2637.676 | 2.31E+00 | −6.57E−01 | 7 | Equal A/Int; Up Islets |
| O95996 | 5161.464 | 1.49E+00 | 9.36E−01 | 7 | Equal A/Int; Up Islets |
| Q14697 | 477.0123 | 1.34E+00 | 1.92E−01 | 7 | Equal A/Int; Up Islets |
| P14209 | 711.8924 | 1.67E+00 | 3.80E−01 | 7 | Equal A/Int; Up Islets |
| P30530 | 412.2115 | 1.01E+00 | −1.29E−01 | 7 | Equal A/Int; Up Islets |
| O43770 | 1085.931 | 1.10E+00 | 5.87E−01 | 7 | Equal A/Int; Up Islets |
| O43852 | 2269.921 | 2.56E+00 | 6.84E−01 | 7 | Equal A/Int; Up Islets |
| O75718 | 592.2327 | 1.34E+00 | −7.52E−01 | 7 | Equal A/Int; Up Islets |
| Q9Y3C0 | 229.7237 | 1.20E+00 | 7.36E−01 | 7 | Equal A/Int; Up Islets |
| P78369 | 1505.471 | 1.14E+00 | −6.65E−01 | 7 | Equal A/Int; Up Islets |
| P08572 | 2389.488 | 2.05E+00 | 2.60E−01 | 7 | Equal A/Int; Up Islets |
| P11802 | 1411.839 | 1.36E+00 | 9.79E−02 | 7 | Equal A/Int; Up Islets |
| Q16555 | 399.8267 | 3.01E+00 | 5.14E−01 | 7 | Equal A/Int; Up Islets |
| P33316 | 318.7836 | 1.61E+00 | 4.23E−01 | 7 | Equal A/Int; Up Islets |
| P07992 | 561.6388 | 1.33E+00 | 1.97E−01 | 7 | Equal A/Int; Up Islets |
| O75636 | 2139.448 | 2.00E+00 | −9.87E−02 | 7 | Equal A/Int; Up Islets |
| Q9UBA6 | 812.6378 | 2.01E+00 | 6.41E−01 | 7 | Equal A/Int; Up Islets |
| Q9Y5P5 | 261.0679 | 1.27E+00 | 2.10E−01 | 7 | Equal A/Int; Up Islets |
| O75293 | 261.3841 | 9.92E−01 | 6.35E−02 | 7 | Equal A/Int; Up Islets |
| P50152 | 283.8702 | 1.28E+00 | −4.53E−01 | 7 | Equal A/Int; Up Islets |
| Q9NX09 | 545.0622 | 1.57E+00 | −4.61E−01 | 7 | Equal A/Int; Up Islets |
| P05204 | 3082.818 | 1.55E+00 | −8.84E−01 | 7 | Equal A/Int; Up Islets |
| Q9P0P2 | 462.1583 | 1.07E+00 | 2.14E−01 | 7 | Equal A/Int; Up Islets |
| Q9NWF8 | 262.6222 | 1.12E+00 | 4.88E−01 | 7 | Equal A/Int; Up Islets |
| Q9NWV4 | 328.2481 | 1.13E+00 | 3.37E−01 | 7 | Equal A/Int; Up Islets |
| Q9P0S8 | 216.4274 | 1.11E+00 | 9.72E−02 | 7 | Equal A/Int; Up Islets |
| Q9BWS9 | 261.7132 | 1.12E+00 | −2.90E−01 | 7 | Equal A/Int; Up Islets |
| Q9BSK0 | 319.1224 | 2.13E+00 | −4.25E−01 | 7 | Equal A/Int; Up Islets |
| Q9Y6M1 | 232.2937 | 1.59E+00 | 1.79E−01 | 7 | Equal A/Int; Up Islets |
| Q9NQX7 | 687.2575 | 1.20E+00 | 8.97E−03 | 7 | Equal A/Int; Up Islets |
| P08648 | 262.0713 | 1.38E+00 | −5.65E−01 | 7 | Equal A/Int; Up Islets |
| O00410 | 384.2402 | 9.47E−01 | −7.35E−01 | 7 | Equal A/Int; Up Islets |
| P07195 | 2599.05 | 2.48E+00 | 2.57E−01 | 7 | Equal A/Int; Up Islets |
| P09382 | 44205.77 | 2.03E+00 | 8.83E−01 | 7 | Equal A/Int; Up Islets |
| O75427 | 316.5457 | 1.63E+00 | 4.03E−01 | 7 | Equal A/Int; Up Islets |
| Q16553 | 619.3701 | 1.14E+00 | 2.13E−01 | 7 | Equal A/Int; Up Islets |
| O75900 | 510.1038 | 1.16E+00 | −3.58E−01 | 7 | Equal A/Int; Up Islets |
| Q9UNF1 | 357.6132 | 1.77E+00 | 1.77E−01 | 7 | Equal A/Int; Up Islets |
| P13995 | 435.6746 | 1.24E+00 | −5.09E−01 | 7 | Equal A/Int; Up Islets |
| O00265 | 267.156 | 9.93E−01 | 3.03E−01 | 7 | Equal A/Int; Up Islets |
| P27361 | 486.849 | 1.01E+00 | 9.38E−02 | 7 | Equal A/Int; Up Islets |
| P24844 | 11793.09 | 2.04E+00 | 4.29E−01 | 7 | Equal A/Int; Up Islets |
| Q9NVD4 | 237.1908 | 1.35E+00 | 1.26E−01 | 7 | Equal A/Int; Up Islets |
| Q9Y617 | 553.0005 | 2.87E+00 | 5.58E−01 | 7 | Equal A/Int; Up Islets |
| O95356 | 901.8229 | 3.26E+00 | −3.05E−01 | 7 | Equal A/Int; Up Islets |
| Q9Y5X6 | 226.0168 | 1.11E+00 | −2.58E−01 | 7 | Equal A/Int; Up Islets |
| Q15113 | 1176.046 | 2.40E+00 | −3.08E−01 | 7 | Equal A/Int; Up Islets |
| O15460 | 483.9569 | 1.53E+00 | 7.25E−01 | 7 | Equal A/Int; Up Islets |
| O43556 | 245.5263 | 1.02E+00 | 3.13E−01 | 7 | Equal A/Int; Up Islets |
| P50454 | 1680.942 | 3.59E+00 | 8.53E−01 | 7 | Equal A/Int; Up Islets |
| Q92853 | 664.6951 | 1.17E+00 | 3.15E−01 | 7 | Equal A/Int; Up Islets |
| Q16658 | 738.014 | 2.49E+00 | 8.66E−01 | 7 | Equal A/Int; Up Islets |
| Q9Y4Y8 | 361.3772 | 1.09E+00 | 1.12E−01 | 7 | Equal A/Int; Up Islets |
| Q9BVH9 | 968.9954 | 1.87E+00 | −1.18E−01 | 7 | Equal A/Int; Up Islets |
| Q9HBB0 | 603.8101 | 1.76E+00 | 1.90E−01 | 7 | Equal A/Int; Up Islets |
| P01033 | 5256.861 | 3.40E+00 | 2.83E−01 | 7 | Equal A/Int; Up Islets |
| Q15582 | 3939.09 | 2.81E+00 | 7.51E−01 | 7 | Equal A/Int; Up Islets |
| Q01995 | 8573.074 | 2.49E+00 | 2.29E−01 | 7 | Equal A/Int; Up Islets |
| Q13641 | 768.6158 | 1.55E+00 | −1.70E−01 | 7 | Equal A/Int; Up Islets |
| P06468 | 4710.469 | 5.28E+00 | 7.96E−01 | 7 | Equal A/Int; Up Islets |
| P51784 | 280.9819 | 1.64E+00 | 2.94E−01 | 7 | Equal A/Int; Up Islets |
| P03996 | 14717.63 | 6.66E+00 | 2.23E+00 | 9 | Increasing A/Int/I |
| P08123 | 5109.251 | 6.29E+00 | 1.63E+00 | 9 | Increasing A/Int/I |
| P24821 | 1215.165 | 2.74E+00 | 1.37E+00 | 9 | Increasing A/Int/I |
| Q9NVA2 | 1568.848 | 3.21E+00 | 1.20E+00 | 9 | Increasing A/Int/I |
| P40261 | 3016.993 | 2.42E+00 | 1.12E+00 | 9 | Increasing A/Int/I |
| Q15063 | 3133.301 | 4.91E+00 | 1.52E+00 | 9 | Increasing A/Int/I |
| P09486 | 22333.48 | 7.30E+00 | 2.77E+00 | 9 | Increasing A/Int/I |
| P35625 | 3637.823 | 5.54E+00 | 1.52E+00 | 9 | Increasing A/Int/I |
| Q9H2L5 | 400.4602 | −1.19E+00 | 9.15E−00 | 19 | Int> (A; I) |
| P15121 | 3354.494 | −3.51E−03 | 1.20E+00 | 19 | Int> (A; I) |
| P08582 | 324.9781 | −1.89E+00 | 9.92E−00 | 19 | Int> (A; I) |
| P20292 | 330.0345 | 1.02E+00 | 2.78E+00 | 19 | Int> (A; I) |
| O15342 | 9572.914 | 2.31E−02 | 1.44E+00 | 19 | Int> (A; I) |
| Q9BXJ0 | 228.0109 | −2.26E−01 | 9.55E−00 | 19 | Int> (A; I) |
| Q9NRJ3 | 948.0274 | 1.61E+00 | 2.87E+00 | 19 | Int> (A; I) |
| P05305 | 1026.992 | −1.10E+00 | 1.06E+00 | 19 | Int> (A; I) |
| P09341 | 2236.622 | 1.09E−01 | 2.35E+00 | 19 | Int> (A; I) |
| Q92730 | 1318.381 | −2.69E−01 | 2.08E+00 | 19 | Int> (A; I) |
| Q9H5K0 | 192.6442 | −2.14E−01 | 9.71E−01 | 19 | Int> (A; I) |
| P18564 | 650.9277 | −1.21E+00 | 1.13E+00 | 19 | Int> (A; I) |
| P14316 | 326.8164 | 1.90E−02 | 9.04E−01 | 19 | Int> (A; I) |
| P40305 | 367.2688 | 8.96E−01 | 2.71E+00 | 19 | Int> (A; I) |
| P05161 | 537.4035 | 1.11E−01 | 1.70E+00 | 19 | Int> (A; I) |
| P42701 | 8218.994 | 1.20E−01 | 4.17E+00 | 19 | Int> (A; I) |
| P09237 | 38905.32 | 1.05E+00 | 3.18E+00 | 19 | Int> (A; I) |
| P16860 | 6171.6 | −1.78E+00 | 1.57E+00 | 19 | Int> (A; I) |
| P21359 | 193.3208 | 2.27E−01 | 1.14E+00 | 19 | Int> (A; I) |
| P02775 | 567.1992 | 2.96E+00 | 5.37E+00 | 19 | Int> (A; I) |
| Q9UDQ9 | 479.5908 | 9.74E−01 | 3.15E+00 | 19 | Int> (A; I) |
| O75635 | 1483.169 | 4.88E−01 | 4.06E+00 | 19 | Int> (A; I) |
| P02735 | 6295.724 | 9.46E−01 | 5.13E+00 | 19 | Int> (A; I) |
| O60635 | 447.5729 | −8.58E−01 | 1.39E+00 | 19 | Int> (A; I) |
| P50591 | 854.3061 | −1.73E−01 | 1.53E+00 | 19 | Int> (A; I) |
| Q03169 | 650.583 | −1.67E−01 | 1.78E+00 | 19 | Int> (A; I) |
| Q9C075 | 1818.182 | −1.08E+00 | 2.31E+00 | 19 | Int> (A; I) |
| P02248 | 369.9913 | −1.33E+00 | 2.52E+00 | 19 | Int> (A; I) |
| O95497 | 887.0948 | 7.24E−01 | 2.57E+00 | 19 | Int> (A; I) |
| Q14202 | 496.3668 | 3.13E−01 | 2.38E+00 | 19 | Int> (A; I) |
| P15514 | 191.2981 | −6.52E−01 | 1.97E+00 | 18 | Off A/I; On Int |
| Q9BZL9 | 140.8098 | −5.80E−01 | 7.42E−01 | 18 | Off A/I; On Int |
| P30991 | 148.3131 | 9.01E−01 | 2.02E+00 | 18 | Off A/I; On Int |
| P29400 | 189.6904 | 1.40E+00 | 2.81E+00 | 18 | Off A/I; On Int |
| Q9UK22 | 141.6466 | 4.59E−01 | 1.31E+00 | 18 | Off A/I; On Int |
| Q16769 | 132.7502 | 3.31E−01 | 1.12E+00 | 18 | Off A/I; On Int |
| P02261 | 151.868 | 9.52E−01 | 2.01E+00 | 18 | Off A/I; On Int |
| P20769 | 126.7136 | 1.87E−02 | 8.00E−01 | 18 | Off A/I; On Int |
| P52945 | 170.7555 | 2.12E−01 | 2.24E+00 | 18 | Off A/I; On Int |
| Q14496 | 161.3017 | −1.45E−02 | 1.53E+00 | 18 | Off A/I; On Int |
| P01583 | 181.5566 | −6.34E−01 | 1.86E+00 | 18 | Off A/I; On Int |
| P01584 | 216.8707 | 2.75E−02 | 1.83E+00 | 18 | Off A/I; On Int |
| Q9GZM1 | 183.8736 | −7.99E−03 | 1.77E+00 | 18 | Off A/I; On Int |
| P25105 | 177.9689 | −2.76E−01 | 1.87E+00 | 18 | Off A/I; On Int |
| P58294 | 152.9437 | 7.82E−01 | 1.59E+00 | 18 | Off A/I; On Int |
| P23471 | 175.0253 | 1.30E−01 | 2.02E+00 | 18 | Off A/I; On Int |
| O88386 | 196.7342 | −8.54E−01 | 1.69E+00 | 18 | Off A/I; On Int |
| P10826 | 143.3844 | 8.31E−01 | 1.83E+00 | 18 | Off A/I; On Int |
| O95786 | 147.8829 | −3.38E−01 | 1.10E+00 | 18 | Off A/I; On Int |
| P02778 | 189.6022 | 4.05E−01 | 2.68E+00 | 18 | Off A/I; On Int |
| P80162 | 164.6594 | 1.26E+00 | 2.52E+00 | 18 | Off A/I; On Int |
| P43005 | 133.1898 | 1.58E−01 | 9.32E−01 | 18 | Off A/I; On Int |
| P01375 | 200.0804 | −8.84E−01 | 1.48E+00 | 18 | Off A/I; On Int |
| Q16890 | 130.885 | −7.92E−02 | 8.84E−01 | 18 | Off A/I; On Int |
| Q9H949 | 136.5769 | −4.92E−01 | 9.64E−01 | 18 | Off A/I; On Int |
| Q9H4G4 | 281.2148 | 2.59E+00 | 1.33E+00 | 8 | Off Acinar; Increasing Int/I |
| Q14040 | 1657.194 | 6.95E+00 | 2.45E+00 | 8 | Off Acinar; Increasing Int/I |
| P12110 | 1362.487 | 5.45E+00 | 8.97E−01 | 8 | Off Acinar; Increasing Int/I |
| P02751 | 1442.812 | 9.88E+00 | 5.10E+00 | 8 | Off Acinar; Increasing Int/I |
| P14652 | 1308.575 | 5.83E+00 | 2.21E+00 | 8 | Off Acinar; Increasing Int/I |
| P08476 | 412.2794 | 3.73E+00 | 2.30E+00 | 8 | Off Acinar; Increasing Int/I |
| P17936 | 563.1937 | 5.09E+00 | 3.19E+00 | 8 | Off Acinar; Increasing Int/I |
| Q9Y4K0 | 953.7036 | 5.06E+00 | 1.77E+00 | 8 | Off Acinar; Increasing Int/I |
| P03956 | 299.8442 | 2.47E+00 | 9.53E−01 | 8 | Off Acinar; Increasing Int/I |
| P39900 | 539.7471 | 6.06E+00 | 4.39E+00 | 8 | Off Acinar; Increasing Int/I |
| P55001 | 1309.293 | 5.06E+00 | 2.40E+00 | 8 | Off Acinar; Increasing Int/I |
| Q9NYR0 | 296.0285 | 4.24E+00 | 2.50E+00 | 8 | Off Acinar; Increasing Int/I |
| O43623 | 1139.155 | 4.97E+00 | 1.59E+00 | 8 | Off Acinar; Increasing Int/I |
| P09936 | 596.7714 | 4.98E+00 | 2.18E+00 | 8 | Off Acinar; Increasing Int/I |
| Q02952 | 232.3779 | 1.14E+00 | 1.81E+00 | 4 | Off Acinar; On Int = I |
| Q99541 | 132.005 | 1.12E+00 | 1.44E+00 | 4 | Off Acinar; On Int = I |
| P54284 | 195.985 | 1.26E+00 | 1.09E+00 | 4 | Off Acinar; On Int = I |
| Q13269 | 170.529 | 1.11E+00 | 9.40E−01 | 4 | Off Acinar; On Int = I |
| Q9NPF2 | 209.7612 | 1.18E+00 | 5.24E−01 | 4 | Off Acinar; On Int = I |
| Q9H7A5 | 161.8637 | 1.08E+00 | 8.58E−01 | 4 | Off Acinar; On Int = I |
| Q9H9Q2 | 128.7341 | 1.09E+00 | 4.14E−01 | 4 | Off Acinar; On Int = I |
| P42771 | 290.1928 | 3.04E+00 | 3.44E+00 | 4 | Off Acinar; On Int = I |
| Q14650 | 242.2271 | 1.28E+00 | 1.04E+00 | 4 | Off Acinar; On Int = I |
| Q96IK6 | 113.265 | −6.73E−01 | −4.89E−01 | 4 | Off Acinar; On Int = I |
| P12034 | 212.2747 | 2.40E+00 | 2.10E+00 | 4 | Off Acinar; On Int = I |
| Q93079 | 281.3281 | 9.07E−01 | 1.56E+00 | 4 | Off Acinar; On Int = I |
| Q9P016 | 290.4476 | 1.69E+00 | 1.50E+00 | 4 | Off Acinar; On Int = I |
| Q9HBI5 | 233.835 | 1.41E+00 | 1.24E+00 | 4 | Off Acinar; On Int = I |
| Q9BY45 | 121.5622 | 1.17E+00 | 4.74E−01 | 4 | Off Acinar; On Int = I |
| P08397 | 196.1361 | 9.07E−01 | 4.38E−01 | 4 | Off Acinar; On Int = I |
| Q9BUV0 | 129.3473 | 1.29E+00 | 1.15E+00 | 4 | Off Acinar; On Int = I |
| Q9H9A2 | 120.7311 | 1.04E+00 | 4.16E−01 | 4 | Off Acinar; On Int = I |
| Q9NUV6 | 134.1021 | 6.44E−01 | 1.03E+00 | 4 | Off Acinar; On Int = I |
| Q9NXF7 | 126.7851 | 1.36E+00 | 1.11E+00 | 4 | Off Acinar; On Int = I |
| Q9H6V0 | 232.5844 | 1.47E+00 | 1.15E+00 | 4 | Off Acinar; On Int = I |
| Q9BUW5 | 151.3887 | 1.34E+00 | 1.08E+00 | 4 | Off Acinar; On Int = I |
| Q9H765 | 177.6046 | 9.58E−01 | 1.05E+00 | 4 | Off Acinar; On Int = I |
| Q14157 | 129.9305 | 6.35E−01 | 6.27E−01 | 4 | Off Acinar; On Int = I |
| Q9Y2D8 | 176.1358 | 1.30E+00 | 1.50E+00 | 4 | Off Acinar; On Int = I |
| Q9H6Z3 | 185.578 | 1.10E+00 | 8.85E−01 | 4 | Off Acinar; On Int = I |
| Q99748 | 173.2957 | 9.94E−01 | 2.74E−01 | 4 | Off Acinar; On Int = I |
| Q9NVD7 | 151.1753 | 1.48E+00 | 7.62E−01 | 4 | Off Acinar; On Int = I |
| O43175 | 136.6151 | 9.61E−01 | 2.64E−01 | 4 | Off Acinar; On Int = I |
| Q9Y253 | 122.2465 | 5.44E−01 | −1.06E−01 | 4 | Off Acinar; On Int = I |
| P24158 | 180.0275 | 9.80E−01 | 1.67E+00 | 4 | Off Acinar; On Int = I |
| Q9U8K7 | 204.6287 | 1.50E+00 | 7.72E−01 | 4 | Off Acinar; On Int = I |
| Q13636 | 121.5169 | 1.15E+00 | 6.30E−01 | 4 | Off Acinar; On Int = I |
| Q9UIC2 | 148.7422 | 1.27E+00 | 1.08E+00 | 4 | Off Acinar; On Int = I |
| Q9NVQ7 | 129.1434 | 9.72E−01 | 7.89E−01 | 4 | Off Acinar; On Int = I |
| O75368 | 167.1823 | 1.08E+00 | 1.19E+00 | 4 | Off Acinar; On Int = I |
| O95863 | 122.817 | 1.04E+00 | 8.06E−01 | 4 | Off Acinar; On Int = I |
| Q9UL01 | 124.5829 | 1.03E+00 | 3.51E−01 | 4 | Off Acinar; On Int = I |
| Q16226 | 364.6675 | 2.25E+00 | 2.03E+00 | 4 | Off Acinar; On Int = I |
| P07996 | 127.0819 | 1.05E+00 | 8.15E−01 | 4 | Off Acinar; On Int = I |
| Q99081 | 182.9347 | 1.19E+00 | 6.86E−01 | 4 | Off Acinar; On Int = I |
| O95922 | 190.8311 | 1.80E+00 | 1.75E+00 | 4 | Off Acinar; On Int = I |
| P15036 | 115.8531 | −8.43E−01 | −3.94E−01 | 4 | Off Acinar; On Int = I |
| O95337 | 172.7489 | 2.03E+00 | 1.24E+00 | 4 | Off Acinar; On Int = I |
| Q9H1B5 | 120.5878 | 1.09E+00 | 3.68E−01 | 4 | Off Acinar; On Int = I |
| P55263 | 637.198 | −1.57E+00 | −3.08E−01 | 15 | On A/Int; Down I |
| P51648 | 1284.046 | −1.95E+00 | −4.93E−01 | 15 | On A/Int; Down I |
| P50995 | 3511.843 | −1.46E+00 | −3.36E−01 | 15 | On A/Int; Down I |
| P12429 | 7424.351 | −2.50E+00 | 1.59E−01 | 15 | On A/Int; Down I |
| P05026 | 3744.962 | −1.00E+00 | 4.16E−01 | 15 | On A/Int; Down I |
| P00751 | 2206.568 | −1.34E+00 | −2.81E−01 | 15 | On A/Int; Down I |
| Q9HA23 | 400.5912 | −1.32E+00 | 1.36E−01 | 15 | On A/Int; Down I |
| Q16170 | 807.5766 | −2.21E+00 | 3.15E−01 | 15 | On A/Int; Down I |
| P25774 | 620.9386 | −1.91E+00 | −1.45E−01 | 15 | On A/Int; Down I |
| P21926 | 2115.74 | −1.27E+00 | 8.00E−01 | 15 | On A/Int; Down I |
| Q9H2A7 | 576.1228 | −1.46E+00 | 3.71E−01 | 15 | On A/Int; Down I |
| O14493 | 1732.749 | −1.26E+00 | 3.70E−01 | 15 | On A/Int; Down I |
| P12277 | 394.4895 | −9.53E−01 | 7.22E−01 | 15 | On A/Int; Down I |
| Q9NYS7 | 397.3278 | −1.50E+00 | −6.35E−01 | 15 | On A/Int; Down I |
| P53355 | 1048.818 | −1.25E+00 | −7.03E−02 | 15 | On A/Int; Down I |
| O15205 | 8102.423 | −1.75E+00 | 1.00E+00 | 15 | On A/Int; Down I |
| Q9UFZ2 | 900.9109 | −2.08E+00 | −3.18E−01 | 15 | On A/Int; Down I |
| P28562 | 1502.864 | −1.46E+00 | −3.09E−01 | 15 | On A/Int; Down I |
| Q16690 | 315.2743 | −1.65E+00 | −6.70E−01 | 15 | On A/Int; Down I |
| Q9UJW0 | 592.1023 | −1.21E+00 | −2.34E−01 | 15 | On A/Int; Down I |
| | 265.0097 | −3.52E−01 | 6.91E−01 | 15 | On A/Int; Down I |
| P00533 | 259.7582 | −5.29E−01 | 3.42E−01 | 15 | On A/Int; Down I |
| Q13113 | 10494.6 | −2.33E+00 | 2.20E−01 | 15 | On A/Int; Down I |
| P28161 | 642.7437 | −2.23E+00 | −5.63E−01 | 15 | On A/Int; Down I |
| Q03013 | 336.469 | −6.60E−01 | 4.81E−01 | 15 | On A/Int; Down I |
| P52594 | 386.6697 | −7.98E−01 | 9.00E−02 | 15 | On A/Int; Down I |
| Q9NWT9 | 729.2943 | −1.53E+00 | −4.89E−01 | 15 | On A/Int; Down I |
| Q9BZR4 | 584.0643 | −2.22E+00 | 1.74E−01 | 15 | On A/Int; Down I |
| Q9BW25 | 291.8079 | −1.07E+00 | 3.76E−01 | 15 | On A/Int; Down I |
| P16144 | 440.0464 | −1.64E+00 | 4.54E−01 | 15 | On A/Int; Down I |
| P10145 | 7330.866 | −1.95E+00 | 9.47E−01 | 15 | On A/Int; Down I |
| Q05084 | 465.8994 | −1.52E+00 | −3.05E−02 | 15 | On A/Int; Down I |
| Q9Y484 | 1429.625 | −5.08E−01 | 5.93E−01 | 15 | On A/Int; Down I |
| P14923 | 1831.613 | −1.11E+00 | 4.07E−01 | 15 | On A/Int; Down I |
| P05783 | 10707.79 | −2.91E+00 | −4.70E−01 | 15 | On A/Int; Down I |
| P08727 | 2484.93 | −2.67E+00 | −5.25E−01 | 15 | On A/Int; Down I |
| P08729 | 8203.912 | −2.62E+00 | −6.19E−02 | 15 | On A/Int; Down I |
| P05787 | 6867.677 | −2.70E+00 | −4.86E−01 | 15 | On A/Int; Down I |
| O14782 | 420.177 | −5.97E−01 | 8.31E−01 | 15 | On A/Int; Down I |
| O00515 | 2140.683 | −2.11E+00 | −8.08E−01 | 15 | On A/Int; Down I |
| P80188 | 4989.308 | −2.98E+00 | 7.93E−01 | 15 | On A/Int; Down I |
| Q9UQ53 | 938.2331 | −1.90E+00 | −6.75E−01 | 15 | On A/Int; Down I |
| P80294 | 300.7815 | −6.50E−01 | 1.57E−01 | 15 | On A/Int; Down I |
| O75394 | 433.794 | −1.05E+00 | 5.49E−01 | 15 | On A/Int; Down I |
| Q9Y376 | 939.6431 | −6.45E−01 | 7.11E−01 | 15 | On A/Int; Down I |
| P15941 | 1357.116 | −2.26E+00 | −8.95E−01 | 15 | On A/Int; Down I |
| Q16301 | 1844.855 | −2.16E+00 | 7.14E−02 | 15 | On A/Int; Down I |
| Q99836 | 871.4196 | −1.40E+00 | 3.15E−02 | 15 | On A/Int; Down I |
| P24001 | 44684.13 | −1.86E+00 | 5.34E−01 | 15 | On A/Int; Down I |
| Q9C002 | 10242.28 | −9.60E−1 | 1.04E+00 | 15 | On A/Int; Down I |
| P25963 | 937.0159 | −6.33E−01 | 4.32E−01 | 15 | On A/Int; Down I |
| P00491 | 523.7383 | −1.71E+00 | −7.13E−01 | 15 | On A/Int; Down I |
| P49763 | 6646.258 | −1.70E+00 | 1.17E+00 | 15 | On A/Int; Down I |
| Q9Y342 | 742.036 | −1.65E+00 | 6.10E−02 | 15 | On A/Int; Down I |
| P43490 | 1546.408 | −1.80E+00 | −3.90E−01 | 15 | On A/Int; Down I |
| Q99988 | 1694.813 | −1.11E+00 | 9.96E−01 | 15 | On A/Int; Down I |
| Q9UJY1 | 1485.828 | −1.75E+00 | 1.77E−01 | 15 | On A/Int; Down I |
| Q9H1C7 | 9771.26 | −1.90E+00 | 3.69E−01 | 15 | On A/Int; Down I |
| P51149 | 3646.784 | −5.94E−01 | 6.18E−1 | 15 | On A/Int; Down I |
| P51151 | 1393.904 | −1.38E+00 | −2.55E−01 | 15 | On A/Int; Down I |
| P15153 | 338.0922 | −8.47E−01 | 1.26E−01 | 15 | On A/Int; Down I |
| P52566 | 708.6827 | −1.49E+00 | −3.24E−01 | 15 | On A/Int; Down I |
| P01011 | 31439.89 | −1.99E+00 | 1.11E−01 | 15 | On A/Int; Down I |
| P05120 | 21999.69 | −3.11E+00 | 6.16E−01 | 15 | On A/Int; Down I |
| O14508 | 1139.68 | −1.85E+00 | −2.42E−02 | 15 | On A/Int; Down I |
| Q9Y6N5 | 659.5107 | −1.54E+00 | 7.99E−02 | 15 | On A/Int; Down I |
| O00161 | 1034.031 | −6.50E−01 | 3.11E−01 | 15 | On A/Int; Down I |
| P31431 | 2026.067 | −1.25E+00 | 5.46E−01 | 15 | On A/Int; Down I |
| O15533 | 299.8834 | −9.48E−01 | 4.38E−01 | 15 | On A/Int; Down I |
| P17987 | 317.0491 | −1.65E+00 | −7.35E−01 | 15 | On A/Int; Down I |
| O14746 | 262.9869 | −8.05E−01 | 4.92E−01 | 15 | On A/Int; Down I |
| P30408 | 13372.16 | −1.46E+00 | 6.34E−02 | 15 | On A/Int; Down I |
| Q9HCN3 | 806.2983 | −1.04E+00 | 9.01E−02 | 15 | On A/Int; Down I |
| Q16149 | 813.3307 | −8.42E−01 | 3.47E−01 | 15 | On A/Int; Down I |
| Q9Y2A9 | 454.8446 | −2.33E+00 | −8.29E−01 | 15 | On A/Int; Down I |
| O60625 | 1914.133 | −2.09E+00 | 1.10E−01 | 15 | On A/Int; Down I |
| P07948 | 375.1466 | −1.59E+00 | −2.12E−01 | 15 | On A/Int; Down I |
| Q9P2N4 | 153.1495 | −1.13E+00 | 5.06E−01 | 14 | On A/Int; Off I |
| P47895 | 1254.954 | −3.09E+00 | 9.27E−01 | 14 | On A/Int; Off I |
| P04233 | 163.6002 | −1.40E+00 | −3.96E−01 | 14 | On A/Int; Off I |
| Q9NZ31 | 285.9642 | −1.33E+00 | 1.18E−01 | 14 | On A/Int; Off I |
| O96002 | 302.4558 | −1.62E+00 | −9.81E−02 | 14 | On A/Int; Off I |
| O94907 | 145.044 | −8.30E−01 | 4.14E−01 | 14 | On A/Int; Off I |
| Q92796 | 133.5184 | −9.32E−01 | −7.56E−02 | 14 | On A/Int; Off I |
| Q13115 | 469.8392 | −1.87E+00 | 4.52E−01 | 14 | On A/Int; Off I |
| P29317 | 209.5143 | −1.70E+00 | 5.78E−02 | 14 | On A/Int; Off I |
| P29323 | 191.9946 | −1.86E+00 | −2.11E−01 | 14 | On A/Int; Off I |
| Q9UKF9 | 430.6372 | −2.56E+00 | −3.92E−02 | 14 | On A/Int; Off I |
| Q9NPD3 | 207.9117 | −2.46E+00 | 1.36E−01 | 14 | On A/Int; Off I |
| O43524 | 177.9385 | −1.35E+00 | −6.35E−01 | 14 | On A/Int; Off I |
| P21217 | 168.8097 | −1.65E+00 | 4.40E−01 | 14 | On A/Int; Off I |
| O75205 | 364.8596 | −2.77E+00 | −7.03E−01 | 14 | On A/Int; Off I |
| O75712 | 260.8655 | −6.93E−01 | 7.72E−01 | 14 | On A/Int; Off I |
| Q92908 | 195.3976 | −1.65E+00 | −5.55E−01 | 14 | On A/Int; Off I |
| O95210 | 228.6851 | −1.56E+00 | −4.60E−01 | 14 | On A/Int; Off I |
| O95395 | 205.5863 | −2.70E+00 | −1.21E−01 | 14 | On A/Int; Off I |
| P48506 | 225.879 | −1.15E+00 | −2.97E−01 | 14 | On A/Int; Off I |
| Q9UI98 | 266.8154 | −1.74E+00 | −8.96E−02 | 14 | On A/Int; Off I |
| Q9H6D8 | 421.5312 | −2.09E+00 | 1.32E−01 | 14 | On A/Int; Off I |
| P05362 | 301.0074 | −2.60E+00 | 4.56E−02 | 14 | On A/Int; Off I |
| Q07627 | 210.3957 | −2.84E+00 | 6.93E−02 | 14 | On A/Int; Off I |
| Q9BYQ7 | 398.2261 | −2.35E+00 | 2.93E−01 | 14 | On A/Int; Off I |
| O75071 | 166.3595 | −1.39E+00 | −5.24E−01 | 14 | On A/Int; Off I |
| Q9UPQ2 | 236.4102 | −7.28E−01 | 6.34E−01 | 14 | On A/Int; Off I |
| O43896 | 134.4385 | −1.12E+00 | 9.16E−02 | 14 | On A/Int; Off I |
| Q13887 | 322.6178 | −2.34E+00 | −3.93E−01 | 14 | On A/Int; Off I |
| O00312 | 128.2701 | −7.73E−01 | 2.20E−02 | 14 | On A/Int; Off I |
| O15264 | 150.0667 | −1.49E+00 | −4.17E−01 | 14 | On A/Int; Off I |
| Q9UHA4 | 195.0133 | −1.89E+00 | −1.41E−01 | 14 | On A/Int; Off I |
| P21397 | 312.5207 | −3.81E+00 | 1.67E−01 | 14 | On A/Int; Off I |
| P55196 | 137.1449 | −1.07E+00 | 2.56E−02 | 14 | On A/Int; Off I |
| Q12965 | 203.3655 | −8.01E−01 | 3.75E−01 | 14 | On A/Int; Off I |
| Q9HBW1 | 300.2834 | −2.27E+00 | −7.79E−02 | 14 | On A/Int; Off I |
| Q9H2W4 | 299.9778 | −2.26E+00 | 3.56E−01 | 14 | On A/Int; Off I |
| Q9NRR3 | 213.1256 | −7.34E−01 | 7.49E−01 | 14 | On A/Int; Off I |
| P23511 | 199.2783 | −8.54E−01 | −8.06E−02 | 14 | On A/Int; Off I |
| Q99650 | 237.114 | −1.26E+00 | −5.33E−01 | 14 | On A/Int; Off I |
| P04085 | 343.2015 | −1.83E+01 | 6.89E−02 | 14 | On A/Int; Off I |
| Q13048 | 145.8869 | −8.25E−01 | 4.41E−01 | 14 | On A/Int; Off I |
| P10586 | 335.8682 | −1.77E+00 | −2.05E−01 | 14 | On A/Int; Off I |
| O95200 | 275.0752 | −2.59E+00 | −4.72E−01 | 14 | On A/Int; Off I |
| Q9NVX8 | 337.8544 | −1.96E+00 | 1.72E−01 | 14 | On A/Int; Off I |
| Q15418 | 246.2347 | −1.93E+00 | 2.68E−01 | 14 | On A/Int; Off I |
| Q15434 | 309.3377 | −1.65E+00 | −4.70E−01 | 14 | On A/Int; Off I |
| Q15437 | 214.4633 | −1.15E+00 | −2.58E−01 | 14 | On A/Int; Off I |
| O60679 | 152.8498 | −1.70E+00 | −5.63E−02 | 14 | On A/Int; Off I |
| Q12890 | 247.1823 | −1.63E+00 | −3.33E−01 | 14 | On A/Int; Off I |
| Q12971 | 224.3379 | −1.35E+00 | 4.11E−01 | 14 | On A/Int; Off I |
| P78556 | 249.7105 | −2.38E+00 | 3.10E−01 | 14 | On A/Int; Off I |
| O75751 | 156.4431 | −1.30E+00 | 5.31E−01 | 14 | On A/Int; Off I |
| Q9UM01 | 172.4611 | −1.85E+00 | 3.66E−01 | 14 | On A/Int; Off I |
| Q9Y5X1 | 189.2981 | −1.01E+00 | 1.30E−01 | 14 | On A/Int; Off I |
| P08842 | 328.904 | −1.68E+00 | −7.62E−02 | 14 | On A/Int; Off I |
| O43760 | 249.1866 | −1.73E+00 | −7.14E−01 | 14 | On A/Int; Off I |
| O75674 | 205.2456 | −2.35E+00 | 4.30E−02 | 14 | On A/Int; Off I |
| P01135 | 145.3108 | −1.86E+00 | −4.29E−01 | 14 | On A/Int; Off I |
| Q9NT70 | 298.7991 | −1.50E+00 | 1.33E−01 | 14 | On A/Int; Off I |
| O60656 | 337.3961 | −4.31E+00 | −3.18E−01 | 14 | On A/Int; Off I |
| Q9NZ42 | 175.1842 | −7.73E−01 | 1.15E−01 | 14 | On A/Int; Off I |
| O76080 | 200.0768 | −2.08E+00 | −5.80E−01 | 14 | On A/Int; Off I |
| Q13015 | 1272.138 | 2.41E+00 | 2.40E+00 | 6 | On Acinar; Up Int = I |
| P04083 | 5883.787 | 2.90E+00 | 2.89E+00 | 6 | On Acinar; Up Int = I |
| O60592 | 2304.427 | 3.56E−01 | 1.07E+00 | 6 | On Acinar; Up Int = I |
| Q07814 | 797.9572 | 1.35E+00 | 1.00E+00 | 6 | On Acinar; Up Int = I |
| Q14201 | 355.9496 | 1.08E+00 | 1.19E+00 | 6 | On Acinar; Up Int = I |
| Q9NR00 | 4453.955 | 2.47E−01 | 1.40E+00 | 6 | On Acinar; Up Int = I |
| Q9BUW7 | 476.5934 | 1.27E+00 | 1.51E+00 | 6 | On Acinar; Up Int = I |
| O95401 | 390.063 | 1.66E+00 | 1.08E+00 | 6 | On Acinar; Up Int = I |
| Q00535 | 392.629 | 6.80E−01 | 8.19E−01 | 6 | On Acinar; Up Int = I |
| P21741 | 9388.855 | 1.71E+00 | 2.36E+00 | 6 | On Acinar; Up Int = I |
| Q9Y291 | 290.8529 | 1.04E+00 | 8.93E−01 | 6 | On Acinar; Up Int = I |
| O75376 | 791.9381 | 9.54E−01 | 1.76E+00 | 6 | On Acinar; Up Int = I |
| P03973 | 2270.718 | 2.81E+00 | 1.80E+00 | 6 | On Acinar; Up Int = I |
| O15427 | 589.0175 | 1.78E+00 | 1.72E+00 | 6 | On Acinar; Up Int = I |
| P16949 | 1781.654 | 1.45E+00 | 1.85E+00 | 6 | On Acinar; Up Int = I |
| Q9NZ86 | 597.7198 | 7.86E−01 | 1.60E+00 | 6 | On Acinar; Up Int = I |
| O00748 | 517.9952 | −2.09E+00 | −2.30E+00 | 20 | Other |
| Q13630 | 388.1959 | −1.28E+00 | −1.12E+00 | 20 | Other |
| P00505 | 369.968 | −9.25E−01 | −4.75E−01 | 20 | Other |
| P50440 | 798.1958 | −2.73E+00 | −2.14E+00 | 20 | Other |
| P29622 | 304.9848 | −8.00E−01 | −1.29E+00 | 20 | Other |
| O14646 | 440.3418 | −1.67E+00 | −2.27E+00 | 20 | Other |
| P48307 | 521.385 | −1.73E+00 | −1.33E+00 | 20 | Other |
| Q15125 | 470.0852 | −1.67E+00 | −1.96E+00 | 20 | Other |
| P19387 | 211.5128 | 5.26E−01 | −3.39E−01 | 20 | Other |
| Q9P0K7 | 245.4643 | 6.73E−01 | 6.26E−01 | 20 | Other |
| P30086 | 465.627 | −1.08E+00 | −4.57E−01 | 20 | Other |
| Q13045 | 476.5407 | −1.02E+00 | −3.14E−01 | 20 | Other |
| O95084 | 552.6166 | −1.80E+00 | −2.15E+00 | 20 | Other |
| P49770 | 536.2071 | −1.15E+00 | −4.19E−01 | 20 | Other |
| Q13948 | 300.5112 | 5.72E−01 | 6.50E−01 | 20 | Other |
| Q30201 | 278.446 | 7.26E−01 | 5.03E−01 | 20 | Other |
| P52895 | 1534.634 | −3.56E+00 | −3.93E+00 | 20 | Other |
| Q9Y5N1 | 356.7517 | −6.26E−01 | −1.38E+00 | 20 | Other |
| P43378 | 724.8175 | −1.88E+00 | −1.61E+00 | 20 | Other |
| P35414 | 474.2345 | −6.49E−01 | −6.12E−01 | 20 | Other |
| Q14802 | 1096.127 | −2.43E+00 | −1.61E+00 | 20 | Other |
| Q9Y266 | 533.6949 | −8.68E−01 | −7.37E−01 | 20 | Other |
| P30043 | 620.0433 | −1.07E+00 | −6.35E−01 | 20 | Other |
| Q9NP73 | 557.2055 | −9.32E−01 | −8.51E−01 | 20 | Other |
| Q12972 | 345.0523 | 7.61E−01 | 5.13E−01 | 20 | Other |
| Q9Y296 | 532.9995 | −7.58E−01 | −8.01E−01 | 20 | Other |
| Q02750 | 586.6635 | −8.76E−01 | −6.72E−01 | 20 | Other |
| P42226 | 619.5568 | −9.87E−01 | −7.92E−01 | 20 | Other |
| P31947 | 1378.205 | −2.70E+00 | −1.99E+00 | 20 | Other |
| P46597 | 1043.272 | −2.25E+00 | −2.35E+00 | 20 | Other |
| O14745 | 997.106 | −1.87E+00 | −1.52E+00 | 20 | Other |
| Q9NS67 | 813.4043 | −1.30E+00 | −1.94E+00 | 20 | Other |
| Q9UKL5 | 829.3129 | −7.16E−01 | −1.68E−01 | 20 | Other |
| Q9UQL3 | 860.8974 | −4.44E−01 | 4.78E−01 | 20 | Other |
| Q9NZZ1 | 488.5944 | 6.37E−01 | 1.78E−01 | 20 | Other |
| Q9Y3Q3 | 504.2124 | 2.63E−01 | −5.68E−01 | 20 | Other |
| Q9NSN3 | 431.8528 | 9.10E−01 | −3.79E−01 | 20 | Other |
| Q9Y5Z0 | 1036.811 | −1.11E+00 | −6.14E−01 | 20 | Other |
| | 699.9713 | 5.20E−01 | 7.41E−01 | 20 | Other |
| O14713 | 643.5596 | 7.25E−01 | 7.42E−01 | 20 | Other |
| P46095 | 1185.918 | −1.62E+00 | −2.34E+00 | 20 | Other |
| Q9BV10 | 1064.815 | −1.18E+00 | −1.43E+00 | 20 | Other |
| Q99653 | 1313.03 | −1.11E+00 | −2.59E−01 | 20 | Other |
| P41584 | 758.0427 | 5.53E−01 | 6.71E−01 | 20 | Other |
| P11940 | 1078.525 | −1.07E+00 | −1.38E+00 | 20 | Other |
| Q00341 | 618.5713 | 6.67E−01 | −2.99E−01 | 20 | Other |
| Q16795 | 1214.409 | −8.58E−01 | −4.15E−01 | 20 | Other |
| Q13082 | 854.4272 | −4.78E−01 | −1.37E+00 | 20 | Other |
| P36405 | 665.1099 | 8.87E−01 | 1.07E−01 | 20 | Other |
| Q9NXH2 | 647.4269 | 8.97E−01 | −1.44E−01 | 20 | Other |
| Q9Y323 | 1148.545 | 2.19E−01 | 9.59E−01 | 20 | Other |
| P48059 | 827.7691 | 8.40E−01 | 7.11E−01 | 20 | Other |
| Q9P0M9 | 846.5801 | 8.23E−01 | 6.93E−01 | 20 | Other |
| Q13813 | 1406.107 | −1.08E+00 | −1.35E+00 | 20 | Other |
| Q9Y379 | 1877.939 | −1.12E+00 | −4.26E−01 | 20 | Other |
| Q13247 | 1041.827 | 7.35E−01 | 7.55E−01 | 20 | Other |
| P14416 | 952.9439 | 6.73E−01 | −1.28E−01 | 20 | Other |
| Q06323 | 1742.377 | −1.05E−01 | 6.55E−01 | 20 | Other |
| Q9NR31 | 1008.646 | 8.82E−01 | 2.36E−01 | 20 | Other |
| P05451 | 26243.72 | −5.76E+00 | −5.48E+00 | 20 | Other |
| P04075 | 2532.66 | −1.62E+00 | −1.51E+00 | 20 | Other |
| Q15904 | 1228.765 | 8.21E−01 | 7.30E−01 | 20 | Other |
| P15559 | 775.5735 | 1.47E+00 | −8.25E−01 | 20 | Other |
| O00303 | 1145.377 | 3.23E−01 | −9.68E−01 | 20 | Other |
| P56705 | 2271.364 | −1.13E+00 | −1.18E+00 | 20 | Other |
| P39210 | 1202.587 | 7.99E−01 | −1.47E−01 | 20 | Other |
| Q9UKL9 | 7016.796 | −3.10E+00 | −2.52E+00 | 20 | Other |
| O43516 | 1444.29 | 7.68E−01 | 3.51E−02 | 20 | Other |
| Q9Y470 | 1503.556 | 6.06E−01 | −2.73E−01 | 20 | Other |
| P29312 | 4076.247 | −1.15E+00 | −1.40E−01 | 20 | Other |
| Q9Y5R8 | 1971.095 | 6.86E−01 | 1.32E−01 | 20 | Other |
| P17676 | 3890.676 | −1.15E+00 | −1.07E+00 | 20 | Other |
| O95362 | 5333.102 | −1.84E+00 | −1.96E+00 | 20 | Other |
| O60417 | 5248.939 | −1.17E+00 | −2.89E−01 | 20 | Other |
| P19623 | 2027.643 | 1.06E+00 | −6.88E−01 | 20 | Other |
| Q9P039 | 3004.627 | 8.96E−01 | −1.87E−01 | 20 | Other |
| Q9BUF5 | 2855.744 | 1.07E+00 | −5.08E−01 | 20 | Other |
| P00995 | 28226.58 | −3.65E+00 | −2.59E+00 | 20 | Other |
| P11142 | 8144.601 | −1.04E+00 | −5.08E−01 | 20 | Other |
| P10620 | 4901.595 | −2.68E−01 | −1.47E+00 | 20 | Other |
| Q13347 | 10168.91 | −1.79E+00 | −1.91E+00 | 20 | Other |
| P15880 | 4127.437 | 4.54E−01 | −1.08E+00 | 20 | Other |
| Q9HCU0 | 4295.784 | 1.19E+00 | −2.72E−01 | 20 | Other |
| Q04941 | 12775.93 | −1.07E+00 | −6.04E−01 | 20 | Other |
| Q9UPI1 | 7300.387 | 9.60E−01 | 8.39E−01 | 20 | Other |
| P13987 | 15673.38 | −7.34E−01 | 1.42E−01 | 20 | Other |
| P15951 | 31332.74 | −2.72E+00 | −3.30E+00 | 20 | Other |
| P04687 | 12871.67 | −4.88E−02 | −1.14E+00 | 20 | Other |
| P04687 | 15834.49 | 6.05E−01 | −1.06E+00 | 20 | Other |
| Q9UI09 | 2210.196 | 7.19E−01 | 1.79E−01 | 3 | Unchanged High |
| Q9Y6H1 | 5191.628 | 3.77E−02 | −4.90E−01 | 3 | Unchanged High |
| Q9P0U1 | 2721.439 | 7.28E−01 | −2.21E−01 | 3 | Unchanged High |
| P80095 | 2471.592 | −1.04E+00 | −2.99E−01 | 3 | Unchanged High |
| Q92747 | 3972.89 | −6.92E−01 | −2.93E−01 | 3 | Unchanged High |
| O15144 | 17538.09 | 3.00E−01 | 6.17E−01 | 3 | Unchanged High |
| O15145 | 2683.638 | −7.73E−01 | 5.78E−02 | 3 | Unchanged High |
| P02570 | 50171.41 | −7.74E−01 | −5.45E−01 | 3 | Unchanged High |
| P02570 | 50643.44 | −5.42E−01 | −6.16E−01 | 3 | Unchanged High |
| P02570 | 16331.85 | −4.70E−01 | −8.03E−01 | 3 | Unchanged High |
| P02571 | 64643.45 | −7.47E−02 | −2.27E−01 | 3 | Unchanged High |
| P12814 | 2400.844 | 1.36E−01 | −1.50E−01 | 3 | Unchanged High |
| O43707 | 4769.196 | −1.00E+00 | −8.78E−01 | 3 | Unchanged High |
| P53999 | 4936.98 | 4.24E−01 | −5.13E−02 | 3 | Unchanged High |
| P18848 | 6749.873 | 9.88E−02 | −4.02E−01 | 3 | Unchanged High |
| P53680 | 2963.423 | 8.63E−01 | 1.67E−01 | 3 | Unchanged High |
| P29274 | 2318.367 | 1.05E−02 | 5.73E−01 | 3 | Unchanged High |
| P54819 | 2788.3 | −7.20E−02 | −2.53E−01 | 3 | Unchanged High |
| Q01518 | 8362.375 | 4.59E−02 | 6.59E−01 | 3 | Unchanged High |
| P32889 | 7056.851 | 3.88E−01 | 2.58E−01 | 3 | Unchanged High |
| P18085 | 7331.81 | 8.04E−01 | 8.93E−01 | 3 | Unchanged High |
| P26437 | 4052.262 | 9.17E−02 | 2.45E−01 | 3 | Unchanged High |
| Q9BZX4 | 2541.066 | 9.21E−02 | 2.44E−01 | 3 | Unchanged High |
| P11766 | 2354.247 | 3.62E−01 | −1.59E−01 | 3 | Unchanged High |
| P05067 | 3649.558 | 4.61E−01 | 5.55E−01 | 3 | Unchanged High |
| Q06481 | 2546.598 | 1.13E−01 | 6.10E−01 | 3 | Unchanged High |
| P07355 | 94734.13 | 3.79E−01 | 5.86E−01 | 3 | Unchanged High |
| P08758 | 5295.976 | 6.69E−01 | −7.32E−02 | 3 | Unchanged High |
| P55064 | 12248.87 | 1.02E−01 | 3.93E−01 | 3 | Unchanged High |
| Q13520 | 2404.397 | 3.12E−02 | −3.25E−02 | 3 | Unchanged High |
| P32391 | 4171.013 | −1.23E−01 | −3.92E−03 | 3 | Unchanged High |
| O43776 | 2811.634 | 1.51E−01 | 2.27E−01 | 3 | Unchanged High |
| P24539 | 3312.717 | 1.50E−01 | 3.86E−01 | 3 | Unchanged High |
| P48201 | 4974.135 | −2.29E−01 | −3.82E−01 | 3 | Unchanged High |
| P05496 | 2112.628 | 1.41E−01 | −3.06E−01 | 3 | Unchanged High |
| Q06055 | 5682.609 | 4.19E−01 | −7.12E−01 | 3 | Unchanged High |
| O75947 | 2355.858 | −5.04E−01 | −8.37E−02 | 3 | Unchanged High |
| P56385 | 2462.524 | −3.60E−02 | −3.62E−01 | 3 | Unchanged High |
| P56134 | 14327.12 | 2.91E−01 | 5.71E−01 | 3 | Unchanged High |
| P18859 | 4633.559 | 4.16E−01 | 3.20E−01 | 3 | Unchanged High |
| O75964 | 2219.11 | −8.99E−03 | 3.78E−01 | 3 | Unchanged High |
| P25705 | 3009.915 | −3.02E−01 | −6.05E−01 | 3 | Unchanged High |
| P06576 | 3305.047 | −6.71E−01 | −3.11E−01 | 3 | Unchanged High |
| P36542 | 2313.989 | −2.54E−02 | −2.91E−01 | 3 | Unchanged High |
| P48047 | 3955.838 | −2.54E−01 | −4.09E−02 | 3 | Unchanged High |
| Q01814 | 3193.535 | 3.84E−02 | 1.05E−02 | 3 | Unchanged High |
| Q99437 | 2817.803 | −2.58E−01 | 6.33E−01 | 3 | Unchanged High |
| P36543 | 2145.908 | −1.60E−01 | 6.57E−01 | 3 | Unchanged High |
| O75348 | 3704.42 | −6.67E−03 | 1.05E+00 | 3 | Unchanged High |
| Q16864 | 10101.59 | 3.03E−01 | 1.30E+00 | 3 | Unchanged High |
| P27449 | 18185.04 | −3.85E−01 | 5.04E−01 | 3 | Unchanged High |
| O00244 | 3435.675 | 4.89E−01 | 3.03E−01 | 3 | Unchanged High |
| O14503 | 4169.761 | −6.13E−01 | −2.20E−01 | 3 | Unchanged High |
| O60238 | 2371.407 | 1.13E+00 | 7.14E−01 | 3 | Unchanged High |
| P30536 | 2405.625 | 2.19E−01 | 5.34E−01 | 3 | Unchanged High |
| P01884 | 68478.91 | −8.47E−01 | 6.88E−01 | 3 | Unchanged High |
| P22004 | 4509.317 | −4.14E−01 | −9.65E−02 | 3 | Unchanged High |
| O95415 | 8032.295 | −4.87E−01 | 5.85E−01 | 3 | Unchanged High |
| O75531 | 2736.992 | 2.03E−01 | 1.11E−01 | 3 | Unchanged High |
| P02593 | 3482.215 | 1.59E+00 | 1.39E+00 | 3 | Unchanged High |
| P07384 | 3078.324 | −5.21E−01 | −5.61E−03 | 3 | Unchanged High |
| P27797 | 11797.33 | 5.89E−01 | −8.16E−02 | 3 | Unchanged High |
| | 23425.99 | 1.00E−01 | 4.75E−01 | 3 | Unchanged High |
| P47756 | 4604.9 | 2.06E−01 | 3.38E−02 | 3 | Unchanged High |
| P15086 | 3273.378 | −8.50E+00 | −8.49E+00 | 3 | Unchanged High |
| P52952 | 2292.94 | −3.25E−01 | −7.19E−01 | 3 | Unchanged High |
| P13862 | 3795.282 | 7.58E−02 | 6.25E−02 | 3 | Unchanged High |
| O14675 | 2092.229 | −1.19E+00 | −6.77E−02 | 3 | Unchanged High |
| P35221 | 3782.772 | −2.08E−01 | 1.51E−01 | 3 | Unchanged High |
| P07858 | 3607.853 | 8.54E−01 | 6.37E−01 | 3 | Unchanged High |
| P48509 | 9901.71 | 2.71E−01 | 9.11E−01 | 3 | Unchanged High |
| P25063 | 15932.79 | −1.36E+00 | −2.28E−01 | 3 | Unchanged High |
| P16070 | 10533.43 | −1.49E+00 | −7.47E−01 | 3 | Unchanged High |
| P08962 | 10307.1 | 9.86E−02 | 3.04E−01 | 3 | Unchanged High |
| O14519 | 4389.995 | 1.71E−01 | −1.90E−02 | 3 | Unchanged High |
| P25763 | 3426.736 | −6.86E−01 | −6.93E−01 | 3 | Unchanged High |
| P49368 | 2484.939 | −8.46E−02 | 2.87E−01 | 3 | Unchanged High |
| P48643 | 2042.12 | −5.20E−01 | 5.49E−02 | 3 | Unchanged High |
| O00299 | 6591.299 | −2.50E−01 | 8.71E−02 | 3 | Unchanged High |
| Q9Y2Q7 | 4610.942 | 5.41E−01 | −1.05E−01 | 3 | Unchanged High |
| P09496 | 6957.341 | 2.26E−02 | 6.86E−01 | 3 | Unchanged High |
| O95832 | 2138.383 | −1.73E+00 | −3.14E−01 | 3 | Unchanged High |
| O95471 | 2587.177 | −9.25E−01 | 2.14E−01 | 3 | Unchanged High |
| P23528 | 16255.64 | −1.68E−01 | 2.22E−01 | 3 | Unchanged High |
| P16989 | 2515.212 | −5.08E−01 | −2.16E−01 | 3 | Unchanged High |
| P38936 | 3777.57 | −8.40E−02 | 2.23E−01 | 3 | Unchanged High |
| Q9UII4 | 31877.4 | 5.99E−01 | 1.87E−01 | 3 | Unchanged High |
| P04080 | 4034.272 | −8.63E−01 | 3.30E−01 | 3 | Unchanged High |
| P21291 | 2109.096 | −2.15E−01 | −5.33E−01 | 3 | Unchanged High |
| O00622 | 2224.339 | 5.25E−01 | −3.50E−01 | 3 | Unchanged High |
| P13073 | 7115.048 | 7.95E−02 | 4.60E−01 | 3 | Unchanged High |
| P12074 | 4434.13 | 3.84E−01 | 8.35E−01 | 3 | Unchanged High |
| Q02221 | 3464.904 | 6.33E−01 | −7.28E−02 | 3 | Unchanged High |
| P14854 | 2344.331 | 2.57E−01 | 4.17E−01 | 3 | Unchanged High |
| P09669 | 2197.58 | −4.21E−01 | −3.08E−01 | 3 | Unchanged High |
| P14406 | 4530.346 | −1.91E−01 | −1.07E−01 | 3 | Unchanged High |
| O14548 | 2556.481 | 4.78E−01 | 1.64E−01 | 3 | Unchanged High |
| P24311 | 4057.995 | 2.38E−01 | 6.75E−01 | 3 | Unchanged High |
| P15954 | 11482.48 | −3.20E−01 | −1.27E−02 | 3 | Unchanged High |
| P10176 | 2750.974 | 1.41E−01 | −6.65E−01 | 3 | Unchanged High |
| O43293 | 2157.783 | −2.80E−01 | −4.55E−01 | 3 | Unchanged High |
| P46966 | 9563.241 | 2.69E−01 | 5.02E−01 | 3 | Unchanged High |
| Q09753 | 4619.611 | −9.19E−01 | 9.07E−01 | 3 | Unchanged High |
| P18282 | 7126.01 | −3.45E−01 | −3.09E−02 | 3 | Unchanged High |
| P07108 | 2422.578 | 2.38E−01 | −5.54E−01 | 3 | Unchanged High |
| P31689 | 2402.398 | −4.77E−01 | 2.32E−01 | 3 | Unchanged High |
| Q9Y5T4 | 2414.457 | 4.46E−02 | 1.17E−01 | 3 | Unchanged High |
| Q9Y463 | 7023.599 | −4.56E−01 | −2.57E−01 | 3 | Unchanged High |
| Q9NP97 | 4534.283 | 3.95E−01 | 2.63E−02 | 3 | Unchanged High |
| Q15701 | 4875.685 | −5.25E−02 | 3.69E−01 | 3 | Unchanged High |
| P78545 | 2673.744 | −1.38E+00 | −4.42E−01 | 3 | Unchanged High |
| O60869 | 5094.856 | −4.31E−01 | 4.19E−02 | 3 | Unchanged High |
| Q14259 | 4241.036 | 5.26E−01 | 1.38E−01 | 3 | Unchanged High |
| P06733 | 9983.774 | −3.33E−01 | −7.50E−01 | 3 | Unchanged High |
| P22712 | 5403.444 | −4.60E−01 | −6.85E−01 | 3 | Unchanged High |
| P04720 | 27648.74 | −6.25E−01 | −6.40E−01 | 3 | Unchanged High |
| P26641 | 8337.901 | 7.58E−01 | −6.91E−02 | 3 | Unchanged High |
| P13639 | 3231.325 | −2.60E−01 | −8.01E−01 | 3 | Unchanged High |
| O15372 | 2398.394 | −2.63E−01 | −5.70E−01 | 3 | Unchanged High |
| O75821 | 3005.577 | 1.48E−01 | −1.70E−01 | 3 | Unchanged High |
| Q64252 | 2141.68 | −3.73E−01 | −8.38E−01 | 3 | Unchanged High |
| O15371 | 3954.374 | −2.64E−01 | −8.46E−02 | 3 | Unchanged High |
| P04765 | 7083.99 | −2.78E−01 | −1.05E−01 | 3 | Unchanged High |
| Q14240 | 2267.015 | 2.68E−01 | 6.61E−02 | 3 | Unchanged High |
| P10159 | 6638.65 | −6.75E−01 | −7.84E−01 | 3 | Unchanged High |
| Q9NQT4 | 2331.056 | 1.35E−01 | 3.04E−01 | 3 | Unchanged High |
| P02794 | 42243.59 | 2.28E−01 | 4.41E−01 | 3 | Unchanged High |
| P02792 | 30568.13 | −1.31E+00 | −5.78E−01 | 3 | Unchanged High |
| Q05472 | 10145.46 | −8.91E−02 | 1.19E−02 | 3 | Unchanged High |
| P20071 | 2009.291 | −9.54E−01 | −5.51E−01 | 3 | Unchanged High |
| P50395 | 2051.41 | 2.24E−03 | −5.38E−01 | 3 | Unchanged High |
| P48507 | 2055.914 | 5.27E+00 | −1.02E+00 | 3 | Unchanged High |
| P36969 | 3787.13 | −1.28E−01 | 5.15E−01 | 3 | Unchanged High |
| P09211 | 6378.198 | −6.52E−01 | 4.40E−01 | 3 | Unchanged High |
| P04406 | 37260.67 | 6.32E−01 | 4.11E−01 | 3 | Unchanged High |
| P04406 | 51621.55 | 6.05E−01 | 6.80E−01 | 3 | Unchanged High |
| P04406 | 15342.7 | 6.61E−01 | 3.07E−01 | 3 | Unchanged High |
| P43304 | 3023.392 | −7.48E−02 | −5.58E−01 | 3 | Unchanged High |
| P41250 | 4736.632 | 5.28E−01 | −2.56E−01 | 3 | Unchanged High |
| P04895 | 8718.93 | 2.32E−01 | 2.61E−01 | 3 | Unchanged High |
| P24522 | 4105.138 | 2.83E−01 | 1.13E+00 | 3 | Unchanged High |
| Q92847 | 2508.414 | −1.23E−01 | −4.29E−01 | 3 | Unchanged High |
| P04901 | 6398.85 | 2.96E−02 | 4.08E−01 | 3 | Unchanged High |
| P25388 | 18939.85 | −2.35E−01 | −5.49E−01 | 3 | Unchanged High |
| P06351 | 2421.658 | −2.61E−01 | 1.80E−01 | 3 | Unchanged High |
| P04792 | 36135.19 | −3.92E−01 | −2.83E−01 | 3 | Unchanged High |
| P11021 | 2976.537 | 3.46E−01 | −1.01E+00 | 3 | Unchanged High |
| P38646 | 2417.791 | −3.61E−01 | −3.87E−01 | 3 | Unchanged High |
| Q9UK76 | 34286.74 | 6.42E−01 | 4.27E−01 | 3 | Unchanged High |
| P09651 | 2231.413 | 1.05E−03 | −6.14E−01 | 3 | Unchanged High |
| Q9Y4J5 | 3320.487 | −2.11E−02 | −6.94E−01 | 3 | Unchanged High |
| P09429 | 2567.093 | 4.67E−01 | −6.06E−01 | 3 | Unchanged High |
| P49773 | 7064.436 | 2.92E−01 | 3.70E−01 | 3 | Unchanged High |
| Q92769 | 2155.575 | −4.56E−01 | −1.43E−01 | 3 | Unchanged High |
| P17693 | 2225.106 | −1.51E−01 | 4.42E−01 | 3 | Unchanged High |
| P17483 | 3095.004 | 5.08E−03 | 2.71E−01 | 3 | Unchanged High |
| Q9UL99 | 2621.286 | −7.27E−01 | −8.21E−01 | 3 | Unchanged High |
| Q9GZT3 | 3797.943 | 3.62E−01 | 1.66E−01 | 3 | Unchanged High |
| Q9NWY5 | 4915.419 | 9.94E−01 | 8.14E−01 | 3 | Unchanged High |
| Q9UI30 | 3819.313 | 5.51E−01 | 4.37E−01 | 3 | Unchanged High |
| Q9BQB6 | 5897.337 | 1.27E+00 | 5.87E−01 | 3 | Unchanged High |
| Q9BWJ5 | 2436.767 | 3.91E−01 | 2.79E−01 | 3 | Unchanged High |
| Q9Y683 | 2699.545 | 2.77E−01 | 1.73E−01 | 3 | Unchanged High |
| Q16665 | 2675.668 | −1.22E+00 | −2.62E−01 | 3 | Unchanged High |
| O75353 | 4129.098 | −5.44E−01 | 4.01E−02 | 3 | Unchanged High |
| Q16270 | 15334.47 | 1.54E+00 | 1.82E+00 | 3 | Unchanged High |
| Q9Y287 | 9139.933 | 5.36E−01 | 6.03E−01 | 3 | Unchanged High |
| P56537 | 2476.062 | −7.26E−01 | 3.32E−01 | 3 | Unchanged High |
| P26006 | 2249.026 | −9.95E−01 | 3.62E−02 | 3 | Unchanged High |
| P05556 | 6216.314 | −5.05E−02 | −3.22E−01 | 3 | Unchanged High |
| Q01628 | 12669.24 | −7.09E−01 | 3.23E−02 | 3 | Unchanged High |
| Q12905 | 2687.538 | −2.30E−01 | −3.32E−01 | 3 | Unchanged High |
| Q13907 | 2362.933 | −2.57E−01 | −8.38E−01 | 3 | Unchanged High |
| P33947 | 2281.692 | 1.24E+00 | −4.12E−01 | 3 | Unchanged High |
| P00338 | 40260.24 | −6.46E−02 | −3.52E−01 | 3 | Unchanged High |
| P08865 | 8180.607 | 2.15E−01 | −4.93E−01 | 3 | Unchanged High |
| Q08380 | 2719.794 | 3.94E−01 | 3.13E−01 | 3 | Unchanged High |
| O14949 | 3377.305 | 3.06E−01 | 2.71E−01 | 3 | Unchanged High |
| Q15012 | 4319.567 | 9.54E−01 | 7.36E−01 | 3 | Unchanged High |
| P14174 | 6337.062 | 2.87E−01 | 2.74E−01 | 3 | Unchanged High |
| P49006 | 8400.725 | 6.17E−01 | 8.22E−01 | 3 | Unchanged High |
| P30463 | 3281.592 | −2.60E−01 | 8.55E−01 | 3 | Unchanged High |
| P10321 | 12044.89 | 1.88E−01 | 9.25E−01 | 3 | Unchanged High |
| P10321 | 13298.19 | 6.28E−01 | 1.59E+00 | 3 | Unchanged High |
| P10321 | 13868.02 | −1.62E−01 | 3.53E−01 | 3 | Unchanged High |
| P13747 | 2460.503 | −3.07E−01 | 1.33E−01 | 3 | Unchanged High |
| Q95HC0 | 4168.835 | −6.21E−01 | −2.90E−01 | 3 | Unchanged High |
| P13640 | 4624.849 | −1.07E+00 | −3.79E−01 | 3 | Unchanged High |
| P80297 | 5142.996 | 1.00E−01 | 1.22E−01 | 3 | Unchanged High |
| P02795 | 7340.215 | 2.85E−01 | 1.48E−01 | 3 | Unchanged High |
| O14880 | 5712.043 | −2.66E−01 | 6.60E−01 | 3 | Unchanged High |
| Q9NYZ2 | 2527.436 | −1.51E−01 | 1.98E−01 | 3 | Unchanged High |
| P26038 | 4212.427 | −1.65E−01 | 1.03E−01 | 3 | Unchanged High |
| O15329 | 6068.269 | 2.62E−01 | 9.31E−02 | 3 | Unchanged High |
| P16475 | 47407.02 | 2.26E−02 | 8.99E−02 | 3 | Unchanged High |
| P19105 | 7764.994 | −1.32E−01 | 4.04E−02 | 3 | Unchanged High |
| Q9UK23 | 2357.087 | 4.14E−01 | 5.05E−01 | 3 | Unchanged High |
| O15239 | 4005.894 | 3.79E−01 | 9.27E−01 | 3 | Unchanged High |
| O00483 | 6613.046 | 8.01E−01 | 8.70E−02 | 3 | Unchanged High |
| O75438 | 2212.932 | 8.04E−01 | 4.13E−01 | 3 | Unchanged High |
| O95168 | 2607.423 | −2.79E−02 | 5.09E−01 | 3 | Unchanged High |
| O95298 | 2544.679 | 5.97E−01 | 5.71E−01 | 3 | Unchanged High |
| O43920 | 8594.08 | 3.61E−01 | 8.85E−01 | 3 | Unchanged High |
| Q13765 | 3363.371 | 4.77E−01 | 2.76E−01 | 3 | Unchanged High |
| Q15843 | 4098.747 | 2.63E−01 | 4.48E−01 | 3 | Unchanged High |
| Q9NX14 | 3902.8 | 5.46E−01 | −1.41E−01 | 3 | Unchanged High |
| Q9Y2R6 | 2113.511 | 4.01E−01 | 3.49E−01 | 3 | Unchanged High |
| Q15668 | 2662.845 | 8.99E−01 | 1.26E+00 | 3 | Unchanged High |
| O14597 | 2697.108 | 4.23E−02 | −4.27E−01 | 3 | Unchanged High |
| P15531 | 7143.349 | 2.36E−01 | 6.07E−02 | 3 | Unchanged High |
| P22392 | 5114.664 | 2.72E−03 | 2.35E−01 | 3 | Unchanged High |
| O00746 | 2927.223 | 9.60E−01 | 3.80E−02 | 3 | Unchanged High |
| O75534 | 3241.269 | −3.46E−01 | 1.20E−02 | 3 | Unchanged High |
| Q9H9A1 | 4076.544 | −9.93E−01 | −8.56E−01 | 3 | Unchanged High |
| Q9NPE3 | 4771.656 | 3.86E−02 | 1.01E+00 | 3 | Unchanged High |
| P54368 | 7292.818 | 1.10E−01 | −6.72E−02 | 3 | Unchanged High |
| Q9UH52 | 2181.482 | −3.10E−01 | −6.73E−03 | 3 | Unchanged High |
| Q15070 | 2031.796 | −5.90E−01 | 2.08E−01 | 3 | Unchanged High |
| Q9H230 | 2199.367 | −3.37E−01 | 8.69E−01 | 3 | Unchanged High |
| O60356 | 2150.631 | 6.27E−01 | 1.25E−01 | 3 | Unchanged High |
| P78337 | 14411.94 | −9.05E−02 | −2.12E−01 | 3 | Unchanged High |
| Q9C086 | 3872.969 | 7.45E−01 | 2.72E−01 | 3 | Unchanged High |
| Q15165 | 2394.717 | 6.78E−01 | 1.49E+00 | 3 | Unchanged High |
| P20962 | 2722.317 | 5.73E−01 | −8.88E−03 | 3 | Unchanged High |
| P23284 | 6683.166 | 7.47E−01 | 1.45E−01 | 3 | Unchanged High |
| Q06830 | 2493.75 | −5.32E−01 | −2.79E−01 | 3 | Unchanged High |
| P32119 | 3130.835 | −1.05E−01 | 8.37E−02 | 3 | Unchanged High |
| P30048 | 2035.218 | −5.34E−01 | −2.66E−01 | 3 | Unchanged High |
| Q13162 | 2822.139 | 4.10E−01 | −4.03E−01 | 3 | Unchanged High |
| P30044 | 2581.24 | −3.63E−01 | 7.94E−01 | 3 | Unchanged High |
| Q13492 | 2414.998 | −5.31E−01 | 3.03E−03 | 3 | Unchanged High |
| P08237 | 2533.875 | 2.00E−02 | −4.94E−01 | 3 | Unchanged High |
| P00558 | 5552.736 | 2.90E−01 | −1.57E−02 | 3 | Unchanged High |
| P18669 | 2477.75 | 6.30E−01 | 5.61E−01 | 3 | Unchanged High |
| Q14801 | 20128.93 | −2.39E−01 | −4.23E−01 | 3 | Unchanged High |
| O14832 | 7819.036 | 1.86E−01 | 8.57E−02 | 3 | Unchanged High |
| P53801 | 4627.881 | −7.19E−02 | 2.80E−01 | 3 | Unchanged High |
| Q9HB21 | 2766.359 | 3.27E−01 | 4.07E−01 | 3 | Unchanged High |
| Q15365 | 3635.431 | −5.49E−01 | −5.60E−01 | 3 | Unchanged High |
| P52433 | 3198.285 | 7.62E−01 | 8.09E−01 | 3 | Unchanged High |
| P52436 | 2489.343 | 3.26E−01 | 2.65E−01 | 3 | Unchanged High |
| Q03052 | 2740.913 | −5.61E−01 | −3.26E−01 | 3 | Unchanged High |
| | 5487.085 | −1.41E+00 | −1.01E−01 | 3 | Unchanged High |
| P40425 | 2958.976 | 2.77E−01 | −9.05E−02 | 3 | Unchanged High |
| Q99471 | 2275.038 | 6.34E−01 | 2.41E−01 | 3 | Unchanged High |
| Q9UHZ2 | 2234.209 | −1.48E−01 | 7.46E−01 | 3 | Unchanged High |
| P07737 | 37970.06 | 2.55E−01 | 7.21E−01 | 3 | Unchanged High |
| Q92740 | 4622.898 | 1.13E−02 | 3.41E−01 | 3 | Unchanged High |
| P07478 | 57240.67 | −5.80E−01 | −8.60E−01 | 3 | Unchanged High |
| Q9UL46 | 3648.995 | −4.41E−01 | 3.06E−01 | 3 | Unchanged High |
| P25786 | 2369.437 | −2.75E−03 | 1.80E−01 | 3 | Unchanged High |
| P20618 | 3082.003 | −7.52E−03 | 3.40E−01 | 3 | Unchanged High |
| P49720 | 2081.991 | 5.06E−01 | 2.89E−01 | 3 | Unchanged High |
| P28070 | 2058.212 | 2.04E−02 | −3.20E−01 | 3 | Unchanged High |
| Q99436 | 3566.368 | −4.44E−01 | −2.78E−01 | 3 | Unchanged High |
| P08129 | 2339.797 | −1.60E−01 | −2.09E−01 | 3 | Unchanged High |
| O15355 | 4605.064 | −6.65E−01 | −4.89E−01 | 3 | Unchanged High |
| P30153 | 3572.89 | −4.81E−01 | 8.58E−02 | 3 | Unchanged High |
| Q15249 | 11954.68 | −1.90E−01 | 3.50E−01 | 3 | Unchanged High |
| Q9NQ11 | 2501.162 | −2.89E−01 | −1.75E−01 | 3 | Unchanged High |
| P41567 | 7666.779 | 2.51E−01 | −1.81E−01 | 3 | Unchanged High |
| Q15181 | 3978.537 | −8.82E−01 | −1.75E−01 | 3 | Unchanged High |
| P14786 | 7787.922 | 5.13E−01 | 3.54E−01 | 3 | Unchanged High |
| P57735 | 3198.699 | −1.29E+00 | 5.47E−01 | 3 | Unchanged High |
| P54725 | 2026.126 | −5.31E−01 | −5.00E−01 | 3 | Unchanged High |
| P54727 | 4809.132 | 3.03E−01 | 4.20E−01 | 3 | Unchanged High |
| O00538 | 2210.191 | −3.44E−01 | −6.90E−02 | 3 | Unchanged High |
| P06749 | 2358.668 | 4.91E−01 | −1.43E−01 | 3 | Unchanged High |
| P08134 | 3943.029 | −5.88E−01 | 2.91E−02 | 3 | Unchanged High |
| | 7033.479 | 7.34E−01 | 2.72E−01 | 3 | Unchanged High |
| P15154 | 10202.94 | −1.11E−01 | −1.59E−01 | 3 | Unchanged High |
| Q15347 | 2244.978 | 6.02E−01 | 4.63E−01 | 3 | Unchanged High |
| O95197 | 3654.06 | 4.61E−02 | −1.20E−01 | 3 | Unchanged High |
| P09455 | 3023.27 | 1.08E−01 | 1.02E+00 | 3 | Unchanged High |
| P52565 | 3169.009 | −1.13E−02 | 1.52E−02 | 3 | Unchanged High |
| P27635 | 10536.25 | −2.72E−01 | −9.17E−01 | 3 | Unchanged High |
| P53025 | 18124.91 | 4.14E−02 | −3.63E−01 | 3 | Unchanged High |
| P39026 | 10427.07 | −4.54E−01 | −1.07E+00 | 3 | Unchanged High |
| P30050 | 33860.13 | 1.10E−02 | −6.45E−01 | 3 | Unchanged High |
| P40429 | 24735.85 | 2.43E−01 | −9.73E−02 | 3 | Unchanged High |
| P50914 | 16402.14 | −2.00E−01 | −3.43E−01 | 3 | Unchanged High |
| P39030 | 9104.589 | 3.62E−01 | −5.43E−02 | 3 | Unchanged High |
| P18621 | 9074.168 | 1.89E−01 | −5.71E−02 | 3 | Unchanged High |
| Q07020 | 12658.45 | 3.60E−01 | −2.56E−01 | 3 | Unchanged High |
| Q02543 | 26796.74 | 1.74E−01 | 1.25E−01 | 3 | Unchanged High |
| P14118 | 5762.718 | −1.70E−01 | −1.72E−01 | 3 | Unchanged High |
| P46778 | 13222.79 | 5.86E−01 | 7.72E−02 | 3 | Unchanged High |
| P35268 | 17168.94 | 2.21E−01 | −5.63E−03 | 3 | Unchanged High |
| P23131 | 22356.23 | 2.95E−01 | −4.24E−02 | 3 | Unchanged High |
| P29316 | 5556.302 | −2.29E−01 | −4.01E−01 | 3 | Unchanged High |
| P38663 | 6488.503 | −5.11E−01 | −3.37E−01 | 3 | Unchanged High |
| P08526 | 9469.252 | −3.28E−01 | −1.29E+00 | 3 | Unchanged High |
| P46776 | 18008.07 | 3.85E−01 | 8.57E−02 | 3 | Unchanged High |
| P46779 | 39577.25 | 1.76E−01 | −3.24E−01 | 3 | Unchanged High |
| P47914 | 2417.223 | −7.81E−01 | −7.73E−01 | 3 | Unchanged High |
| P39023 | 12535.38 | 8.18E−02 | −3.36E−01 | 3 | Unchanged High |
| P04645 | 17188.03 | 2.83E−01 | −5.20E−01 | 3 | Unchanged High |
| P12947 | 10441.63 | 3.69E−01 | −1.18E−01 | 3 | Unchanged High |
| P02433 | 25982.45 | 4.33E−01 | 1.01E−01 | 3 | Unchanged High |
| P49207 | 12661.96 | −6.27E−03 | −1.99E−01 | 3 | Unchanged High |
| P42766 | 10527.37 | 2.75E−02 | 2.47E−03 | 3 | Unchanged High |
| P18077 | 11356.13 | −7.27E−01 | −4.50E−01 | 3 | Unchanged High |
| P09896 | 9742.196 | 5.22E−01 | 6.44E−02 | 3 | Unchanged High |
| P09896 | 5203.379 | −1.39E−01 | 1.54E−01 | 3 | Unchanged High |
| P02403 | 39386.47 | 3.73E−01 | 4.95E−01 | 3 | Unchanged High |
| P12751 | 18651.98 | 9.74E−02 | −1.05E−01 | 3 | Unchanged High |
| P23411 | 6091.715 | 5.54E−01 | 1.12E−01 | 3 | Unchanged High |
| P02404 | 19427.01 | 6.94E−01 | 5.96E−01 | 3 | Unchanged High |
| P36578 | 11723.95 | −4.59E−01 | −4.72E−01 | 3 | Unchanged High |
| P28751 | 36614.61 | 2.00E−01 | −3.32E−02 | 3 | Unchanged High |
| P46777 | 10411.06 | −2.13E−01 | −4.35E−01 | 3 | Unchanged High |
| Q02878 | 11094.61 | −6.48E−01 | −1.12E+00 | 3 | Unchanged High |
| P18124 | 18885.03 | −1.20E−01 | −4.18E−01 | 3 | Unchanged High |
| P11518 | 10736.39 | −3.38E−01 | −7.00E−01 | 3 | Unchanged High |
| P25120 | 22208.81 | 7.08E−02 | −3.67E−01 | 3 | Unchanged High |
| P32969 | 32766.75 | 3.70E−01 | 1.56E−01 | 3 | Unchanged High |
| P46783 | 7540.695 | 1.13E−02 | 3.89E−02 | 3 | Unchanged High |
| P04643 | 14209.6 | 2.07E−02 | 2.14E−01 | 3 | Unchanged High |
| P25398 | 13018.69 | −3.95E−01 | −6.11E−01 | 3 | Unchanged High |
| Q02546 | 13946.74 | 1.32E−01 | −1.48E−01 | 3 | Unchanged High |
| P11174 | 21408.92 | 1.30E−01 | −1.24E−01 | 3 | Unchanged High |
| P39027 | 17912.92 | 3.81E−01 | −1.53E−01 | 3 | Unchanged High |
| P17008 | 22257.3 | −1.20E−01 | −7.62E−01 | 3 | Unchanged High |
| P08708 | 12443.09 | 1.36E−01 | −8.07E−01 | 3 | Unchanged High |
| P25232 | 15089.7 | 6.78E−01 | 6.84E−03 | 3 | Unchanged High |
| P17075 | 24400.98 | 5.16E−01 | −1.43E−01 | 3 | Unchanged High |
| P35265 | 10847.43 | −6.38E−01 | −5.69E−01 | 3 | Unchanged High |
| P39028 | 12545.6 | 2.51E−01 | −1.72E−01 | 3 | Unchanged High |
| P16632 | 12699.84 | −8.96E−02 | 1.53E−02 | 3 | Unchanged High |
| P25111 | 5677.337 | −2.92E−01 | −1.55E−01 | 3 | Unchanged High |
| P02383 | 25499.92 | −4.27E−01 | 2.42E−01 | 3 | Unchanged High |
| P42677 | 16927.4 | 2.15E−01 | −4.51E−01 | 3 | Unchanged High |
| P14798 | 3073.17 | −2.52E−01 | −4.77E−02 | 3 | Unchanged High |
| P25112 | 30318.47 | −9.09E−02 | 7.66E−02 | 3 | Unchanged High |
| P30054 | 21630.2 | 4.69E−01 | 1.79E−01 | 3 | Unchanged High |
| P23396 | 4754.218 | −9.85E−02 | −1.07E+00 | 3 | Unchanged High |
| P49241 | 7316.667 | 2.64E−01 | −2.06E−01 | 3 | Unchanged High |
| P12750 | 19282.5 | 1.36E−01 | −2.87E−01 | 3 | Unchanged High |
| P22090 | 2580.924 | 7.98E−02 | −1.72E−01 | 3 | Unchanged High |
| P46782 | 14598.96 | 1.70E−01 | −4.85E−01 | 3 | Unchanged High |
| P10660 | 37695.24 | −1.94E−01 | −8.30E−01 | 3 | Unchanged High |
| P23821 | 16494.24 | −4.00E−01 | −2.19E−01 | 3 | Unchanged High |
| P09058 | 16406.16 | 3.97E−01 | −3.76E−01 | 3 | Unchanged High |
| P46761 | 5285.193 | −1.21E−01 | −6.63E−01 | 3 | Unchanged High |
| P46781 | 11278.36 | 7.93E−01 | 1.38E−01 | 3 | Unchanged High |
| P46781 | 5525.883 | −6.62E−01 | −2.34E+00 | 3 | Unchanged High |
| P05387 | 15695.14 | 9.78E−02 | −3.87E−01 | 3 | Unchanged High |
| P05388 | 16430.54 | 2.05E−01 | −4.64E−01 | 3 | Unchanged High |
| P05386 | 2413.78 | 4.92E−01 | −2.46E−01 | 3 | Unchanged High |
| Q9Y254 | 3408.892 | 3.74E−01 | 7.15E−01 | 3 | Unchanged High |
| Q9NWJ8 | 2666.66 | 8.79E−02 | 9.99E−01 | 3 | Unchanged High |
| P08206 | 40017.88 | −2.78E−01 | 3.72E−01 | 3 | Unchanged High |
| P31949 | 8466.714 | −7.17E−01 | 3.20E−01 | 3 | Unchanged High |
| P06703 | 3188.753 | −7.04E−01 | 2.60E−01 | 3 | Unchanged High |
| P55735 | 2016.062 | 4.79E−01 | 2.32E−01 | 3 | Unchanged High |
| P38384 | 6908.432 | 3.60E−01 | 4.67E−01 | 3 | Unchanged High |
| Q9NZJ3 | 2230.091 | −1.14E+00 | −2.60E−01 | 3 | Unchanged High |
| O15532 | 3189.642 | 5.49E−01 | 1.50E−01 | 3 | Unchanged High |
| Q13501 | 2517.543 | −1.47E+00 | −1.82E−02 | 3 | Unchanged High |
| P35237 | 5105.477 | −5.17E−01 | 9.60E−01 | 3 | Unchanged High |
| P05121 | 6972.284 | 1.04E+00 | 7.09E−01 | 3 | Unchanged High |
| Q00587 | 2393.46 | −2.79E−01 | 9.80E−02 | 3 | Unchanged High |
| Q9H299 | 15134.74 | −6.90E−02 | 2.43E−01 | 3 | Unchanged High |
| P37108 | 28082.56 | −3.96E−04 | 5.49E−01 | 3 | Unchanged High |
| P49458 | 2551.928 | 7.45E−01 | 4.93E−01 | 3 | Unchanged High |
| P43308 | 4898.581 | 4.81E−01 | 1.31E−01 | 3 | Unchanged High |
| P51571 | 2349.29 | 3.65E−01 | 1.77E−01 | 3 | Unchanged High |
| O00422 | 2396.002 | −2.67E−01 | 5.83E−02 | 3 | Unchanged High |
| O75918 | 6955.245 | 4.19E−01 | 2.42E−01 | 3 | Unchanged High |
| P13500 | 21330.26 | −9.59E−01 | 1.16E+00 | 3 | Unchanged High |
| Q15356 | 2570.901 | 3.96E−02 | −3.52E−01 | 3 | Unchanged High |
| P14648 | 2021.429 | 1.39E+00 | 1.64E+00 | 3 | Unchanged High |
| P12236 | 6107.995 | −2.58E−01 | −6.32E−01 | 3 | Unchanged High |
| Q00325 | 4200.626 | −4.25E−01 | −4.37E−01 | 3 | Unchanged High |
| P32745 | 4340.964 | 2.11E−01 | 2.39E−02 | 3 | Unchanged High |
| P30626 | 2323.149 | 2.67E−01 | 5.59E−01 | 3 | Unchanged High |
| Q01826 | 30645.09 | −1.27E−01 | 6.42E−01 | 3 | Unchanged High |
| P21673 | 10275.93 | −4.74E−01 | 9.80E−01 | 3 | Unchanged High |
| P34991 | 2820.643 | −3.59E−01 | −3.80E−02 | 3 | Unchanged High |
| P23246 | 2834.056 | 3.13E−01 | −1.32E−01 | 3 | Unchanged High |
| P35716 | 4001.241 | −1.24E+00 | −4.71E−02 | 3 | Unchanged High |
| P35713 | 2565.998 | −3.48E−01 | 2.66E−01 | 3 | Unchanged High |
| Q06945 | 2049.214 | −4.91E−01 | −5.59E−02 | 3 | Unchanged High |
| P00441 | 4202.954 | −4.55E−01 | 1.51E−01 | 3 | Unchanged High |
| P50502 | 2400.085 | 3.12E−02 | 1.35E−01 | 3 | Unchanged High |
| Q9Y5Y6 | 2183.041 | −6.58E−01 | 1.93E−01 | 3 | Unchanged High |
| P17600 | 2067.417 | 3.03E−02 | 1.74E−01 | 3 | Unchanged High |
| O43759 | 11775.53 | −5.29E−01 | −4.33E−01 | 3 | Unchanged High |
| Q12962 | 4992.528 | −5.83E−01 | −2.26E−01 | 3 | Unchanged High |
| Q15763 | 2600.459 | 5.12E−01 | 5.16E−01 | 3 | Unchanged High |
| P10599 | 9965.777 | −8.40E−01 | 5.00E−02 | 3 | Unchanged High |
| P13472 | 58508.68 | 2.08E−01 | 6.61E−01 | 3 | Unchanged High |
| P01253 | 21783.42 | −5.12E−01 | 1.66E−01 | 3 | Unchanged High |
| Q01085 | 2312.331 | 3.74E−01 | −3.47E−01 | 3 | Unchanged High |
| Q15370 | 3505.384 | 5.83E−01 | 6.05E−01 | 3 | Unchanged High |
| O43680 | 2209.014 | 7.67E−01 | 3.49E−03 | 3 | Unchanged High |
| P21980 | 2827.774 | −7.53E−01 | 1.58E−01 | 3 | Unchanged High |
| P29401 | 2671.504 | −2.63E−01 | −6.75E−01 | 3 | Unchanged High |
| O60739 | 3315.099 | 5.34E−01 | 3.50E−01 | 3 | Unchanged High |
| Q9NS69 | 3062.809 | 3.29E−01 | −2.89E−01 | 3 | Unchanged High |
| P00938 | 6622.243 | 5.50E−01 | 2.97E−02 | 3 | Unchanged High |
| P07226 | 2897.509 | 5.11E−01 | −1.97E−01 | 3 | Unchanged High |
| P04687 | 18992.31 | 1.42E−01 | −4.84E−01 | 3 | Unchanged High |
| P04687 | 15193.76 | 5.59E−01 | −9.60E−01 | 3 | Unchanged High |
| Q13509 | 2394.89 | −3.36E−01 | 3.56E−01 | 3 | Unchanged High |
| P04350 | 5149.498 | −1.14E+00 | −7.75E−01 | 3 | Unchanged High |
| O75347 | 3390.719 | 1.27E−01 | 3.77E−01 | 3 | Unchanged High |
| O75509 | 3007.078 | −2.55E−01 | 1.12E+00 | 3 | Unchanged High |
| P13693 | 49731.11 | 2.81E−01 | 6.10E−01 | 3 | Unchanged High |
| P14625 | 3049.346 | 1.17E+00 | −8.69E−02 | 3 | Unchanged High |
| P42655 | 5956.713 | −2.44E−01 | −2.01E−01 | 3 | Unchanged High |
| Q04917 | 7111.932 | −9.18E−01 | −8.07E−02 | 3 | Unchanged High |
| P29312 | 4275.506 | −1.37E+00 | −3.29E−01 | 3 | Unchanged High |
| P29312 | 2406.497 | −1.59E+00 | −2.01E+00 | 3 | Unchanged High |
| O14957 | 3351.45 | 2.88E−01 | 5.49E−01 | 3 | Unchanged High |
| P14793 | 2840.732 | −1.18E−01 | −1.03E+00 | 3 | Unchanged High |
| P02248 | 3779.352 | 5.92E−01 | 6.86E−01 | 3 | Unchanged High |
| O76069 | 2068.646 | 1.52E−01 | −1.46E−02 | 3 | Unchanged High |
| Q9BZL1 | 6239.513 | 2.83E−01 | 1.07E+00 | 3 | Unchanged High |
| P08670 | 10813.94 | 1.58E+00 | 5.53E−01 | 3 | Unchanged High |
| P45880 | 6347.014 | −5.58E−01 | 5.49E−02 | 3 | Unchanged High |
| Q9Y277 | 2216.75 | 4.66E−01 | 2.48E−01 | 3 | Unchanged High |
| P17861 | 2347.333 | −7.36E−01 | −1.08E+00 | 3 | Unchanged High |
| O43670 | 3226.643 | −7.15E−02 | −7.67E−02 | 3 | Unchanged High |
| Q07352 | 5601.518 | −2.98E−01 | −8.49E−02 | 3 | Unchanged High |
| Q15942 | 2387.987 | −2.86E−02 | −3.09E−01 | 3 | Unchanged High |
| Q16659 | 128.6073 | −5.32E−01 | −3.51E−02 | 1 | Unchanged Low |
| O75324 | 134.7009 | −3.97E−01 | 3.88E−01 | 1 | Unchanged Low |
| Q9BYP8 | 150.0893 | −8.18E−01 | −2.33E−02 | 1 | Unchanged Low |
| P55291 | 128.8583 | −6.62E−01 | −3.16E−01 | 1 | Unchanged Low |
| P49768 | 117.5823 | −3.51E−01 | −4.04E−02 | 1 | Unchanged Low |
| P55107 | 127.9343 | −1.89E−01 | 5.54E−01 | 1 | Unchanged Low |
| P17082 | 236.7776 | −2.10E+00 | −1.30E+00 | 1 | Unchanged Low |
| P23634 | 185.4081 | −8.30E−01 | 6.21E−01 | 1 | Unchanged Low |
| P78347 | 118.6963 | −5.87E−01 | −5.52E−01 | 1 | Unchanged Low |
| P09912 | 444.3716 | −8.61E−01 | 2.51E+00 | 1 | Unchanged Low |
| Q9H2F5 | 114.5773 | −3.30E−01 | −1.48E−01 | 1 | Unchanged Low |
| O14811 | 128.8019 | −5.85E−01 | −2.02E−01 | 1 | Unchanged Low |
| O60942 | 125.1735 | −7.98E−01 | −8.65E−01 | 1 | Unchanged Low |
| Q9Y5Y3 | 145.8365 | −7.75E−01 | −1.12E−01 | 1 | Unchanged Low |
| P11908 | 147.2722 | −7.05E−01 | 6.46E−02 | 1 | Unchanged Low |
| Q92851 | 115.0308 | −4.15E−01 | −3.42E−01 | 1 | Unchanged Low |
| Q9BQE5 | 119.8218 | −1.06E−01 | 4.26E−01 | 1 | Unchanged Low |
| P55327 | 116.9522 | −4.16E−01 | −2.77E−01 | 1 | Unchanged Low |
| P28332 | 114.0924 | −4.79E−01 | −5.43E−01 | 1 | Unchanged Low |
| O95861 | 115.4153 | −3.27E−01 | −1.44E−01 | 1 | Unchanged Low |
| Q9NXJ5 | 125.322 | −2.59E−01 | 3.05E−01 | 1 | Unchanged Low |
| Q15642 | 121.6641 | −2.41E−01 | 2.30E−01 | 1 | Unchanged Low |
| O94997 | 138.8592 | −7.69E−01 | −3.17E−01 | 1 | Unchanged Low |
| O95453 | 115.7147 | −4.52E−01 | −4.16E−01 | 1 | Unchanged Low |
| Q9H9C5 | 121.5247 | −4.07E−01 | −1.05E−01 | 1 | Unchanged Low |
| P10451 | 123.2388 | −8.94E−01 | −1.31E+00 | 1 | Unchanged Low |
| P53992 | 113.9998 | −3.89E−01 | −3.53E−01 | 1 | Unchanged Low |
| Q9NQW1 | 123.8833 | −1.18E−01 | 5.02E−01 | 1 | Unchanged Low |
| Q01449 | 150.5611 | −7.97E−01 | −7.94E−02 | 1 | Unchanged Low |
| Q9NX46 | 123.8664 | −8.73E−02 | 5.37E−01 | 1 | Unchanged Low |
| P53609 | 123.8135 | −4.41E−01 | −1.26E−01 | 1 | Unchanged Low |
| Q15029 | 115.9932 | −2.10E−01 | 7.28E−02 | 1 | Unchanged Low |
| P50458 | 122.9669 | −5.00E−01 | −2.84E−01 | 1 | Unchanged Low |
| P33764 | 152.5858 | −5.43E−01 | 4.36E−01 | 1 | Unchanged Low |
| Q9P289 | 117.7841 | −3.68E−01 | −1.98E−01 | 1 | Unchanged Low |
| P52429 | 118.2956 | −3.15E−01 | −6.96E−02 | 1 | Unchanged Low |
| P54578 | 124.2132 | −6.33E−01 | −5.71E−01 | 1 | Unchanged Low |
| Q13219 | 123.4254 | −1.21E−01 | 4.44E−01 | 1 | Unchanged Low |
| O60547 | 119.8044 | −5.05E−01 | −4.43E−01 | 1 | Unchanged Low |
| P23297 | 124.9008 | −4.83E−01 | −2.10E−01 | 1 | Unchanged Low |
| Q9H5J8 | 137.2762 | −1.05E+00 | −1.24E+00 | 1 | Unchanged Low |
| Q14493 | 128.4702 | −2.84E−01 | 2.85E−01 | 1 | Unchanged Low |
| Q9NZU0 | 219.1816 | −1.45E+00 | −8.65E−02 | 1 | Unchanged Low |
| P80370 | 114.6185 | −6.18E−01 | −1.00E+00 | 1 | Unchanged Low |
| Q9NYV4 | 113.4542 | −3.01E−01 | −2.60E−01 | 1 | Unchanged Low |
| P09016 | 156.5829 | −7.31E−01 | 1.50E−01 | 1 | Unchanged Low |
| Q13873 | 115.6624 | −5.77E−01 | −8.45E−01 | 1 | Unchanged Low |
| O14878 | 116.398 | −2.58E−01 | −5.89E−02 | 1 | Unchanged Low |
| P52848 | 118.3793 | −6.70E−01 | −9.77E−01 | 1 | Unchanged Low |
| Q14848 | 121.4689 | −2.50E−01 | 1.27E−01 | 1 | Unchanged Low |
| O00204 | 135.653 | −8.31E−01 | −6.71E−01 | 1 | Unchanged Low |
| P35610 | 112.1404 | −4.60E−01 | −7.08E−01 | 1 | Unchanged Low |
| Q04760 | 114.7132 | −3.43E−01 | −3.12E−01 | 1 | Unchanged Low |
| P51690 | 119.2772 | −7.59E−01 | −1.22E+00 | 1 | Unchanged Low |
| P26012 | 128.4899 | −8.19E−02 | 6.19E−01 | 1 | Unchanged Low |
| Q14978 | 117.4911 | −1.61E−01 | 1.61E−01 | 1 | Unchanged Low |
| O00764 | 135.073 | −6.14E−01 | −1.92E−01 | 1 | Unchanged Low |
| Q9Y6Q5 | 120.3004 | −5.12E−01 | −4.85E−01 | 1 | Unchanged Low |
| O95350 | 122.6314 | −3.90E−01 | −1.26E−01 | 1 | Unchanged Low |
| P49748 | 122.4441 | −2.09E−01 | 2.23E−01 | 1 | Unchanged Low |
| P08100 | 115.4338 | −2.58E−01 | −1.10E−01 | 1 | Unchanged Low |
| Q15477 | 131.0636 | 3.14E−02 | 8.58E−01 | 1 | Unchanged Low |
| O43520 | 131.8072 | −9.26E−01 | −1.12E+00 | 1 | Unchanged Low |
| Q13039 | 120.383 | −7.45E−01 | −1.13E+00 | 1 | Unchanged Low |
| O15291 | 185.7368 | −1.40E+00 | −6.52E−01 | 1 | Unchanged Low |
| Q9NR71 | 122.091 | −5.43E−02 | 4.84E−01 | 1 | Unchanged Low |
| O60884 | 188.5763 | −1.08E+00 | 1.05E−01 | 1 | Unchanged Low |
| Q9BYE0 | 113.6134 | −2.87E−01 | −2.39E−01 | 1 | Unchanged Low |
| Q9Y272 | 124.0354 | 5.29E−02 | 7.13E−01 | 1 | Unchanged Low |
| Q9BUR5 | 140.65 | 4.24E−01 | 1.61E+00 | 1 | Unchanged Low |
| O60859 | 115.3513 | −2.39E−01 | −8.28E−02 | 1 | Unchanged Low |
| Q9GZU1 | 144.4516 | 3.01E−01 | 1.51E+00 | 1 | Unchanged Low |
| P19878 | 310.3051 | −1.56E+00 | 7.31E−01 | 1 | Unchanged Low |
| Q13277 | 136.1937 | −1.72E−01 | 6.41E−01 | 1 | Unchanged Low |
| | 120.0361 | −3.14E−01 | −7.76E−02 | 1 | Unchanged Low |
| P32456 | 118.8515 | −5.54E−02 | 3.76E−01 | 1 | Unchanged Low |
| Q9H4M9 | 134.9992 | −5.00E−01 | 1.54E−02 | 1 | Unchanged Low |
| Q9BTX7 | 126.203 | −1.37E−01 | 4.40E−01 | 1 | Unchanged Low |
| Q9NVN2 | 114.476 | −2.31E−01 | −1.17E−01 | 1 | Unchanged Low |
| Q99758 | 163.1362 | −1.18E+00 | −7.14E−01 | 1 | Unchanged Low |
| P43694 | 124.6996 | −2.93E−02 | 5.77E−01 | 1 | Unchanged Low |
| Q13825 | 123.0884 | −2.25E−01 | 1.83E−01 | 1 | Unchanged Low |
| Q92550 | 138.2365 | −9.02E−01 | −8.24E−01 | 1 | Unchanged Low |
| Q9UJS0 | 121.1782 | −1.72E−01 | 2.17E−01 | 1 | Unchanged Low |
| Q99611 | 134.292 | −6.73E−01 | −3.99E−01 | 1 | Unchanged Low |
| P17275 | 112.2701 | −3.60E−01 | −5.18E−01 | 1 | Unchanged Low |
| Q13477 | 114.7415 | −2.71E−01 | −2.11E−01 | 1 | Unchanged Low |
| Q14692 | 117.1898 | −1.12E−01 | 1.95E−01 | 1 | Unchanged Low |
| P49908 | 116.7316 | −4.08E−01 | −4.41E−01 | 1 | Unchanged Low |
| P54803 | 113.8221 | −2.99E−01 | −3.11E−01 | 1 | Unchanged Low |
| P50991 | 138.5613 | −6.06E−01 | −1.23E−01 | 1 | Unchanged Low |
| P35548 | 137.5011 | −7.83E−01 | −5.57E−01 | 1 | Unchanged Low |
| P11309 | 113.8227 | −3.63E−01 | −4.62E−01 | 1 | Unchanged Low |
| O75570 | 117.597 | −2.81E−01 | −1.31E−01 | 1 | Unchanged Low |
| Q9NY61 | 116.8527 | −1.81E−01 | 4.36E−02 | 1 | Unchanged Low |
| Q9BTY8 | 112.876 | −3.94E−01 | −5.87E−01 | 1 | Unchanged Low |
| Q9NWS2 | 143.0354 | −7.85E−01 | −3.89E−01 | 1 | Unchanged Low |
| O00180 | 119.3824 | −2.85E−01 | −8.14E−02 | 1 | Unchanged Low |
| O15382 | 112.8868 | −3.64E−01 | −5.19E−01 | 1 | Unchanged Low |
| Q9Y2Z1 | 153.067 | −5.57E−01 | 3.28E−01 | 1 | Unchanged Low |
| Q9Y315 | 117.8557 | −3.48E−01 | −2.89E−01 | 1 | Unchanged Low |
| Q9Y592 | 115.3792 | −1.53E−01 | 2.56E−02 | 1 | Unchanged Low |
| Q9NYT0 | 124.8364 | −1.07E−01 | 4.10E−01 | 1 | Unchanged Low |
| Q9Y343 | 116.6438 | −2.04E−01 | −3.60E−02 | 1 | Unchanged Low |
| Q9BVI4 | 123.6298 | −3.96E−01 | −1.88E−01 | 1 | Unchanged Low |
| P30622 | 119.7985 | −1.27E−01 | 2.15E−01 | 1 | Unchanged Low |
| Q08345 | 212.6497 | −1.32E+00 | −1.35E−02 | 1 | Unchanged Low |
| P33176 | 114.5075 | −2.05E−01 | −1.33E−01 | 1 | Unchanged Low |
| P55273 | 126.8971 | 1.08E−01 | 8.01E−01 | 1 | Unchanged Low |
| Q9NW92 | 150.3287 | −7.21E−01 | −9.00E−02 | 1 | Unchanged Low |
| Q9NZC3 | 121.8015 | 2.21E−02 | 5.23E−01 | 1 | Unchanged Low |
| P15172 | 116.7347 | −3.05E−01 | −2.72E−01 | 1 | Unchanged Low |
| P27144 | 116.7128 | −1.40E−01 | 6.42E−02 | 1 | Unchanged Low |
| O60508 | 129.259 | −4.81E−01 | −2.09E−01 | 1 | Unchanged Low |
| Q14012 | 118.8965 | −8.21E−02 | 2.46E−01 | 1 | Unchanged Low |
| P98179 | 113.6891 | −3.79E−01 | −5.89E−01 | 1 | Unchanged Low |
| Q13223 | 124.3969 | −4.78E−01 | −3.79E−01 | 1 | Unchanged Low |
| P55198 | 138.7788 | −7.46E−01 | −5.69E−01 | 1 | Unchanged Low |
| P46459 | 114.1635 | −3.66E−01 | −5.37E−01 | 1 | Unchanged Low |
| P34969 | 169.3507 | −1.02E+00 | −2.70E−01 | 1 | Unchanged Low |
| Q14667 | 145.1883 | 2.58E−02 | 1.07E+00 | 1 | Unchanged Low |
| Q92830 | 114.5758 | −2.63E−01 | −2.83E−01 | 1 | Unchanged Low |
| | 124.4395 | −5.87E−02 | 4.45E−01 | 1 | Unchanged Low |
| O43252 | 163.4169 | −8.50E−01 | −5.02E−02 | 1 | Unchanged Low |
| Q9NS93 | 160.8518 | −3.55E−01 | 7.76E−01 | 1 | Unchanged Low |
| O60616 | 215.0608 | −2.81E+00 | −3.35E+00 | 1 | Unchanged Low |
| P02261 | 112.4051 | −4.26E−01 | −7.87E−01 | 1 | Unchanged Low |
| Q14701 | 178.2467 | −1.40E−01 | 1.37E+00 | 1 | Unchanged Low |
| Q9P025 | 134.6437 | −5.67E−01 | −2.44E−01 | 1 | Unchanged Low |
| P06702 | 138.5985 | −4.19E−01 | 1.70E−01 | 1 | Unchanged Low |
| Q9Y5Q9 | 155.7377 | −3.56E−01 | 6.68E−01 | 1 | Unchanged Low |
| P19075 | 113.7491 | −2.54E−01 | −3.30E−01 | 1 | Unchanged Low |
| O60905 | 119.4574 | −2.73E−02 | 3.27E−01 | 1 | Unchanged Low |
| Q13322 | 114.7351 | −1.53E−01 | −7.64E−02 | 1 | Unchanged Low |
| Q9UQ09 | 134.0152 | −8.06E−01 | −8.89E−01 | 1 | Unchanged Low |
| P57053 | 140.5964 | −6.07E−02 | 8.11E−01 | 1 | Unchanged Low |
| Q92876 | 145.5667 | −8.57E−01 | −5.97E−01 | 1 | Unchanged Low |
| P09960 | 130.2822 | 1.78E−01 | 9.44E−01 | 1 | Unchanged Low |
| Q9HBH1 | 112.7249 | −3.40E−01 | −6.01E−01 | 1 | Unchanged Low |
| P55082 | 118.1545 | −1.54E−01 | 2.87E−02 | 1 | Unchanged Low |
| Q9NVH6 | 121.828 | −6.79E−01 | −1.10E+00 | 1 | Unchanged Low |
| O95249 | 119.983 | 2.10E−02 | 4.12E−01 | 1 | Unchanged Low |
| Q13891 | 118.5076 | −2.14E−01 | −8.18E−02 | 1 | Unchanged Low |
| Q9BZM5 | 162.5485 | −2.91E−01 | 8.73E−01 | 1 | Unchanged Low |
| O94913 | 131.7517 | −9.13E−01 | −1.36E+00 | 1 | Unchanged Low |
| Q12987 | 119.3623 | −2.72E−01 | −1.81E−01 | 1 | Unchanged Low |
| Q92499 | 136.6656 | −6.91E−01 | −5.13E−01 | 1 | Unchanged Low |
| Q13686 | 119.4385 | 5.32E−02 | 4.42E−01 | 1 | Unchanged Low |
| P43250 | 114.2814 | −1.37E−01 | −9.10E−02 | 1 | Unchanged Low |
| Q9UMX0 | 124.0398 | −1.55E−01 | 2.04E−01 | 1 | Unchanged Low |
| O94905 | 114.8261 | −2.22E−01 | −2.57E−01 | 1 | Unchanged Low |
| Q9UHK0 | 118.2396 | −5.22E−01 | −8.46E−01 | 1 | Unchanged Low |
| P25189 | 117.3068 | −1.13E−02 | 2.52E−01 | 1 | Unchanged Low |
| Q10567 | 168.4738 | −9.28E−01 | −1.62E−01 | 1 | Unchanged Low |
| O15230 | 120.9363 | −2.63E−02 | 3.35E−01 | 1 | Unchanged Low |
| P52294 | 152.7331 | −7.01E−01 | −7.70E−02 | 1 | Unchanged Low |
| O60668 | 114.8794 | −1.68E−01 | −1.56E−01 | 1 | Unchanged Low |
| P11688 | 121.0017 | −3.05E−01 | −2.19E−01 | 1 | Unchanged Low |
| Q9NWB6 | 119.9579 | −6.66E−01 | −1.22E+00 | 1 | Unchanged Low |
| Q9H028 | 118.4626 | 3.52E−02 | 3.57E−01 | 1 | Unchanged Low |
| Q9UMZ2 | 118.5628 | −1.26E−01 | −1.32E−02 | 1 | Unchanged Low |
| P24752 | 146.8035 | −9.71E−01 | −9.23E−01 | 1 | Unchanged Low |
| P11230 | 122.607 | 5.50E−02 | 5.11E−01 | 1 | Unchanged Low |
| O14977 | 119.7626 | −1.21E−01 | 1.06E−01 | 1 | Unchanged Low |
| | 364.6946 | −3.41E−01 | 2.59E+00 | 1 | Unchanged Low |
| P56937 | 137.3284 | −1.04E+00 | −1.61E+00 | 1 | Unchanged Low |
| O75299 | 117.1803 | −3.61E−02 | 1.78E−01 | 1 | Unchanged Low |
| P16219 | 118.69 | 1.17E−02 | 3.16E−01 | 1 | Unchanged Low |
| Q9NZ48 | 137.2614 | −2.80E−02 | 7.46E−01 | 1 | Unchanged Low |
| Q9P163 | 123.4514 | 1.27E−01 | 6.51E−01 | 1 | Unchanged Low |
| Q9BVT8 | 127.7419 | −2.95E−01 | 1.76E−02 | 1 | Unchanged Low |
| Q99933 | 115.0469 | −1.15E−01 | −5.98E−02 | 1 | Unchanged Low |
| O14645 | 160.4204 | −5.71E−01 | 3.31E−01 | 1 | Unchanged Low |
| Q9BRR9 | 115.1711 | −1.23E−01 | −7.10E−02 | 1 | Unchanged Low |
| Q9Y3E7 | 157.1807 | −5.18E−01 | 3.54E−01 | 1 | Unchanged Low |
| P46976 | 118.5706 | −2.06E−01 | −1.21E−01 | 1 | Unchanged Low |
| O75928 | 127.4786 | 1.64E−01 | 8.06E−01 | 1 | Unchanged Low |
| Q9NX63 | 131.146 | −1.55E−01 | 3.68E−01 | 1 | Unchanged Low |
| Q9UBS0 | 150.9199 | −7.54E−01 | −2.71E−01 | 1 | Unchanged Low |
| Q15185 | 117.54 | −6.12E−01 | −1.23E+00 | 1 | Unchanged Low |
| O60516 | 120.8526 | 8.76E−02 | 4.97E−01 | 1 | Unchanged Low |
| Q16651 | 190.1399 | −1.59E+00 | −1.41E+00 | 1 | Unchanged Low |
| P48788 | 114.1376 | −1.30E−01 | −1.40E−01 | 1 | Unchanged Low |
| Q01968 | 125.3025 | −6.07E−01 | −8.17E−01 | 1 | Unchanged Low |
| Q9H6Z6 | 117.2667 | −5.26E−02 | 1.25E−01 | 1 | Unchanged Low |
| Q9NQ55 | 117.676 | −1.41E−01 | −3.50E−02 | 1 | Unchanged Low |
| O95359 | 119.4624 | 3.79E−02 | 3.62E−01 | 1 | Unchanged Low |
| P01189 | 115.5442 | −3.84E−01 | −6.82E−01 | 1 | Unchanged Low |
| Q9Y525 | 123.6546 | −9.82E−02 | 2.42E−01 | 1 | Unchanged Low |
| Q9UHC9 | 118.9021 | 4.79E−02 | 3.53E−01 | 1 | Unchanged Low |
| Q9HBT2 | 118.9743 | −4.05E−01 | −5.90E−01 | 1 | Unchanged Low |
| O00219 | 141.641 | −1.01E+00 | −1.34E+00 | 1 | Unchanged Low |
| P22735 | 113.1407 | −2.95E−01 | −5.89E−01 | 1 | Unchanged Low |
| O15388 | 121.3043 | 8.41E−02 | 4.82E−01 | 1 | Unchanged Low |
| O95389 | 113.4959 | −2.73E−01 | −5.21E−01 | 1 | Unchanged Low |
| Q06190 | 116.2494 | −2.69E−01 | −3.98E−01 | 1 | Unchanged Low |
| P50226 | 120.0792 | −4.01E−01 | −5.52E−01 | 1 | Unchanged Low |
| Q16632 | 116.1383 | −1.11E−01 | −6.14E−02 | 1 | Unchanged Low |
| P54762 | 129.1191 | 5.96E−02 | 6.48E−01 | 1 | Unchanged Low |
| Q15269 | 114.654 | −1.61E−01 | −2.25E−01 | 1 | Unchanged Low |
| O14495 | 113.9808 | −1.10E−01 | −1.44E−01 | 1 | Unchanged Low |
| Q9P0U0 | 121.9267 | −4.47E−01 | −5.91E−01 | 1 | Unchanged Low |
| P50120 | 187.0028 | −1.05E+00 | −9.36E−02 | 1 | Unchanged Low |
| Q9UHR0 | 124.9147 | 1.91E−03 | 4.33E−01 | 1 | Unchanged Low |
| Q13977 | 132.4319 | 9.09E−02 | 7.75E−01 | 1 | Unchanged Low |
| P51692 | 112.9412 | −1.78E−01 | −3.43E−01 | 1 | Unchanged Low |
| P51164 | 120.3347 | −2.82E−01 | −2.81E−01 | 1 | Unchanged Low |
| Q9NXH3 | 117.6836 | −1.35E−02 | 1.70E−01 | 1 | Unchanged Low |
| | 116.8658 | −8.33E−03 | 1.51E−01 | 1 | Unchanged Low |
| P16435 | 118.227 | 2.71E−02 | 2.62E−01 | 1 | Unchanged Low |
| | 121.7814 | −4.80E−02 | 2.36E−01 | 1 | Unchanged Low |
| Q02080 | 117.0502 | −3.03E−01 | −4.69E−01 | 1 | Unchanged Low |
| Q15334 | 115.3049 | −1.34E−01 | −1.62E−01 | 1 | Unchanged Low |
| P10636 | 123.46 | −4.39E−02 | 2.94E−01 | 1 | Unchanged Low |
| Q9Y4J6 | 117.8247 | −3.05E−01 | −4.43E−01 | 1 | Unchanged Low |
| Q9UHY7 | 118.5336 | 4.73E−02 | 3.00E−01 | 1 | Unchanged Low |
| Q14147 | 116.4348 | −5.95E−02 | 1.76E−02 | 1 | Unchanged Low |
| O95880 | 128.5046 | −5.75E−01 | −6.88E−01 | 1 | Unchanged Low |
| Q15013 | 138.5918 | 1.13E−01 | 9.22E−01 | 1 | Unchanged Low |
| Q9P1E2 | 119.6869 | −2.93E−01 | −3.69E−01 | 1 | Unchanged Low |
| P07203 | 115.8519 | −9.06E−02 | −8.19E−02 | 1 | Unchanged Low |
| Q13206 | 115.4153 | −3.12E−01 | −6.03E−01 | 1 | Unchanged Low |
| O15085 | 115.3055 | −1.05E−01 | −1.37E−01 | 1 | Unchanged Low |
| P48426 | 169.6778 | −1.04E+00 | −5.16E−01 | 1 | Unchanged Low |
| Q9NRQ2 | 118.4782 | −1.56E−01 | −1.23E−01 | 1 | Unchanged Low |
| O43171 | 117.8862 | −1.61E−01 | −1.60E−01 | 1 | Unchanged Low |
| Q9HB90 | 127.5658 | −2.29E−01 | 3.71E−02 | 1 | Unchanged Low |
| Q9H733 | 121.334 | −8.84E−04 | 2.71E−01 | 1 | Unchanged Low |
| P43353 | 118.734 | −3.21E−01 | −4.90E−01 | 1 | Unchanged Low |
| Q15053 | 123.496 | 3.09E−02 | 3.91E−01 | 1 | Unchanged Low |
| P22301 | 132.021 | −6.22E−01 | −7.00E−01 | 1 | Unchanged Low |
| Q9BW47 | 131.1841 | −1.47E−01 | 2.92E−01 | 1 | Unchanged Low |
| P21917 | 141.2355 | −4.80E−01 | −6.96E−02 | 1 | Unchanged Low |
| Q9Y6K5 | 127.6931 | −1.59E−01 | 1.56E−01 | 1 | Unchanged Low |
| Q9Y2A4 | 127.5854 | −2.54E−01 | −3.82E−02 | 1 | Unchanged Low |
| Q16880 | 119.9407 | 1.56E−02 | 2.33E−01 | 1 | Unchanged Low |
| O95747 | 116.7318 | 1.08E−02 | 1.12E−01 | 1 | Unchanged Low |
| P25106 | 120.5368 | 1.82E−01 | 5.42E−01 | 1 | Unchanged Low |
| | 120.6924 | 1.55E−01 | 4.98E−01 | 1 | Unchanged Low |
| P45984 | 113.914 | −2.86E−01 | −6.64E−01 | 1 | Unchanged Low |
| Q9NPF7 | 131.5891 | 2.11E−01 | 8.77E−01 | 1 | Unchanged Low |
| Q9UH94 | 120.3231 | −2.95E−01 | −4.04E−01 | 1 | Unchanged Low |
| Q16559 | 118.9991 | −1.99E−01 | −2.45E−01 | 1 | Unchanged Low |
| Q9UGH0 | 123.6748 | 2.16E−01 | 6.75E−01 | 1 | Unchanged Low |
| Q15544 | 118.4317 | −3.46E−02 | 6.82E−02 | 1 | Unchanged Low |
| O00628 | 122.588 | −4.45E−01 | −6.89E−01 | 1 | Unchanged Low |
| O43241 | 114.422 | −1.10E−01 | −2.46E−01 | 1 | Unchanged Low |
| Q9Y6N1 | 114.0678 | −1.46E−01 | −3.44E−01 | 1 | Unchanged Low |
| Q9UHX5 | 122.8348 | 2.26E−01 | 6.60E−01 | 1 | Unchanged Low |
| Q01664 | 150.0321 | −1.22E+00 | −2.01E+00 | 1 | Unchanged Low |
| Q9BUI1 | 117.3611 | 3.55E−02 | 1.53E−01 | 1 | Unchanged Low |
| Q9HBL5 | 118.5457 | 1.46E−01 | 3.93E−01 | 1 | Unchanged Low |
| Q9NXV2 | 129.6462 | −1.38E−01 | 2.16E−01 | 1 | Unchanged Low |
| Q15424 | 114.2781 | −2.29E−01 | −5.42E−01 | 1 | Unchanged Low |
| P26440 | 136.2534 | −6.33E−01 | −6.38E−01 | 1 | Unchanged Low |
| O60264 | 117.7402 | 1.24E−01 | 3.22E−01 | 1 | Unchanged Low |
| Q15286 | 145.6 | −2.62E−01 | 4.06E−01 | 1 | Unchanged Low |
| Q9UGK0 | 196.7868 | −1.39E+00 | −8.21E−01 | 1 | Unchanged Low |
| Q92980 | 146.2003 | −3.00E−01 | 3.50E−01 | 1 | Unchanged Low |
| Q99726 | 116.3247 | 3.39E−02 | 9.75E−02 | 1 | Unchanged Low |
| P01111 | 115.8629 | −9.70E−02 | −1.90E−01 | 1 | Unchanged Low |
| Q92574 | 117.8303 | 7.50E−02 | 2.20E−01 | 1 | Unchanged Low |
| P16106 | 122.8016 | −2.20E−01 | −1.98E−01 | 1 | Unchanged Low |
| Q9NWX5 | 115.5043 | −2.67E−02 | −7.26E−02 | 1 | Unchanged Low |
| Q9Y6B7 | 168.0108 | −9.09E−01 | −3.57E−01 | 1 | Unchanged Low |
| Q9Y4F1 | 118.0819 | 9.52E−02 | 2.55E−01 | 1 | Unchanged Low |
| Q06710 | 121.7641 | −4.93E−02 | 1.03E−01 | 1 | Unchanged Low |
| Q9H6N1 | 190.8582 | −8.84E−01 | 1.73E−01 | 1 | Unchanged Low |
| Q9NSY1 | 117.0914 | 5.50E−02 | 1.38E−01 | 1 | Unchanged Low |
| Q14106 | 122.8358 | −2.11E−02 | 1.87E−01 | 1 | Unchanged Low |
| Q9UK11 | 122.8322 | −3.47E−02 | 1.60E−01 | 1 | Unchanged Low |
| P08069 | 133.4883 | 1.29E−01 | 7.42E−01 | 1 | Unchanged Low |
| Q9H6F5 | 116.1638 | −5.11E−02 | −1.10E−01 | 1 | Unchanged Low |
| Q14373 | 168.3955 | −4.39E−01 | 5.51E−01 | 1 | Unchanged Low |
| P20393 | 120.571 | 1.20E−01 | 3.67E−01 | 1 | Unchanged Low |
| P36915 | 116.2706 | −5.41E−02 | −1.18E−01 | 1 | Unchanged Low |
| O95109 | 121.0452 | −3.01E−01 | −4.79E−01 | 1 | Unchanged Low |
| Q9NVS2 | 119.5497 | −1.67E−02 | 7.31E−02 | 1 | Unchanged Low |
| Q92950 | 118.6125 | 8.72E−02 | 2.34E−01 | 1 | Unchanged Low |
| Q14977 | 127.3783 | 1.26E−01 | 5.62E−01 | 1 | Unchanged Low |
| P16106 | 123.5385 | 3.04E−01 | 7.45E−01 | 1 | Unchanged Low |
| Q9Y3C1 | 141.934 | −3.11E−01 | 1.72E−01 | 1 | Unchanged Low |
| P37287 | 119.4716 | −1.77E−01 | −2.87E−01 | 1 | Unchanged Low |
| Q9UIG5 | 122.443 | 1.94E−01 | 5.25E−01 | 1 | Unchanged Low |
| Q9UNE2 | 117.0111 | 8.86E−02 | 1.62E−01 | 1 | Unchanged Low |
| Q14123 | 123.5304 | −4.75E−02 | 1.21E−01 | 1 | Unchanged Low |
| Q16206 | 130.1671 | 1.15E−02 | 4.26E−01 | 1 | Unchanged Low |
| Q9NWT1 | 116.4708 | 1.77E−02 | −2.15E−03 | 1 | Unchanged Low |
| Q12756 | 162.7767 | −2.36E−01 | 7.47E−01 | 1 | Unchanged Low |
| P28288 | 117.9674 | −2.56E−01 | −5.51E−01 | 1 | Unchanged Low |
| P05198 | 117.2266 | 2.59E−02 | 3.77E−02 | 1 | Unchanged Low |
| Q9H0V9 | 115.9248 | −1.31E−01 | −3.47E−01 | 1 | Unchanged Low |
| Q92984 | 198.9799 | −1.13E+00 | −2.23E−01 | 1 | Unchanged Low |
| O95498 | 116.8572 | 1.02E−01 | 1.64E−01 | 1 | Unchanged Low |
| Q9H1E1 | 119.4295 | 1.08E−01 | 2.62E−01 | 1 | Unchanged Low |
| Q9NV88 | 117.172 | 1.18E−01 | 2.04E−01 | 1 | Unchanged Low |
| Q9Y310 | 117.0556 | −2.52E−02 | −8.10E−02 | 1 | Unchanged Low |
| P48382 | 143.9771 | −6.98E−01 | −6.47E−01 | 1 | Unchanged Low |
| P52738 | 136.2205 | −2.10E−01 | 1.71E−01 | 1 | Unchanged Low |
| Q9NRW1 | 132.4668 | 2.32E−01 | 8.29E−01 | 1 | Unchanged Low |
| O15298 | 165.1797 | −1.18E+00 | −1.27E+00 | 1 | Unchanged Low |
| Q9NUN5 | 116.1573 | 4.62E−02 | 1.41E−02 | 1 | Unchanged Low |
| Q07699 | 115.8757 | 2.19E−02 | −4.76E−02 | 1 | Unchanged Low |
| P27701 | 114.2624 | 2.60E−02 | −1.05E−01 | 1 | Unchanged Low |
| Q9UER5 | 118.2694 | 1.74E−01 | 3.24E−01 | 1 | Unchanged Low |
| Q8TAC6 | 124.5089 | 1.42E−02 | 2.28E−01 | 1 | Unchanged Low |
| Q9NRW9 | 120.1901 | −2.03E−01 | −3.76E−01 | 1 | Unchanged Low |
| O95147 | 166.9415 | −6.17E−01 | 1.37E−01 | 1 | Unchanged Low |
| Q13895 | 116.9004 | 1.37E−01 | 1.98E−01 | 1 | Unchanged Low |
| P31645 | 140.7231 | 5.19E−02 | 7.19E−01 | 1 | Unchanged Low |
| P51511 | 122.1739 | −4.82E−02 | 2.16E−02 | 1 | Unchanged Low |
| Q15154 | 128.7076 | −1.05E−01 | 1.25E−01 | 1 | Unchanged Low |
| Q00007 | 116.7703 | 1.35E−01 | 1.82E−01 | 1 | Unchanged Low |
| P55822 | 128.0275 | −1.46E−01 | 1.76E−02 | 1 | Unchanged Low |
| Q14442 | 118.0476 | −2.44E−02 | −8.61E−02 | 1 | Unchanged Low |
| P54792 | 119.4338 | −8.94E−02 | −1.70E−01 | 1 | Unchanged Low |
| P42336 | 121.01 | 2.37E−01 | 4.98E−01 | 1 | Unchanged Low |
| Q9UMQ3 | 132.6879 | 2.59E−01 | 8.49E−01 | 1 | Unchanged Low |
| Q9UPY5 | 120.9226 | −5.44E−01 | −1.32E+00 | 1 | Unchanged Low |
| P19113 | 116.6743 | 9.22E−02 | 8.57E−02 | 1 | Unchanged Low |
| O95394 | 118.8198 | −6.50E−02 | −1.52E−01 | 1 | Unchanged Low |
| P22557 | 150.2281 | −7.60E−01 | −6.46E−01 | 1 | Unchanged Low |
| Q9Y3A2 | 131.6512 | 1.36E−01 | 6.16E−01 | 1 | Unchanged Low |
| Q9H5J4 | 113.8461 | −1.76E−01 | −6.34E−01 | 1 | Unchanged Low |
| P16083 | 124.3296 | −3.73E−01 | −6.50E−01 | 1 | Unchanged Low |
| Q99819 | 117.2457 | −7.30E−02 | −2.42E−01 | 1 | Unchanged Low |
| O75569 | 118.9057 | 2.54E−01 | 4.47E−01 | 1 | Unchanged Low |
| O15038 | 113.8294 | −6.45E−02 | −3.74E−01 | 1 | Unchanged Low |
| Q9HBH5 | 120.3437 | 2.30E−01 | 4.48E−01 | 1 | Unchanged Low |
| P41231 | 127.3275 | −1.39E−01 | −1.84E−02 | 1 | Unchanged Low |
| O15397 | 117.4304 | 6.43E−02 | 3.71E−02 | 1 | Unchanged Low |
| Q9UDY8 | 118.129 | 6.73E−02 | 6.76E−02 | 1 | Unchanged Low |
| O95819 | 122.7769 | 3.76E−01 | 7.55E−01 | 1 | Unchanged Low |
| Q12999 | 114.8641 | 6.17E−02 | −6.74E−02 | 1 | Unchanged Low |
| P25440 | 114.7701 | 4.08E−02 | −1.16E−01 | 1 | Unchanged Low |
| P20827 | 392.8203 | −1.31E+00 | 1.35E+00 | 1 | Unchanged Low |
| Q9H652 | 118.9065 | −1.05E−01 | −2.80E−01 | 1 | Unchanged Low |
| P31483 | 118.2227 | 1.93E−01 | 2.87E−01 | 1 | Unchanged Low |
| Q9BSM8 | 180.6391 | −2.98E−01 | 8.82E−01 | 1 | Unchanged Low |
| P36897 | 114.8546 | 6.00E−02 | −9.83E−02 | 1 | Unchanged Low |
| Q9UMR9 | 191.0958 | −1.08E+00 | −3.82E−01 | 1 | Unchanged Low |
| Q92560 | 127.8687 | 2.53E−01 | 6.72E−01 | 1 | Unchanged Low |
| Q9NWU1 | 124.8325 | 2.76E−01 | 6.26E−01 | 1 | Unchanged Low |
| Q92696 | 205.3234 | −8.83E−01 | 2.91E−01 | 1 | Unchanged Low |
| O95236 | 119.1528 | 1.80E−01 | 2.87E−01 | 1 | Unchanged Low |
| Q13202 | 131.9045 | −2.77E−01 | −1.88E−01 | 1 | Unchanged Low |
| P11277 | 116.0084 | 9.99E−02 | 2.26E−02 | 1 | Unchanged Low |
| O95397 | 121.1071 | 2.77E−01 | 5.16E−01 | 1 | Unchanged Low |
| P05423 | 116.0734 | −1.24E−01 | −4.65E−01 | 1 | Unchanged Low |
| Q14338 | 133.9938 | 2.78E−01 | 8.63E−01 | 1 | Unchanged Low |
| Q9BYI8 | 125.1864 | 4.25E−01 | 8.67E−01 | 1 | Unchanged Low |
| Q06203 | 147.3953 | −8.38E−01 | −1.04E+00 | 1 | Unchanged Low |
| Q9NZ33 | 132.4428 | 8.83E−02 | 5.12E−01 | 1 | Unchanged Low |
| P04196 | 117.5882 | 6.06E−02 | −7.63E−03 | 1 | Unchanged Low |
| O96029 | 138.7174 | −7.31E−01 | −1.11E+00 | 1 | Unchanged Low |
| Q01196 | 232.9078 | −1.86E+00 | −1.69E+00 | 1 | Unchanged Low |
| Q9UHL4 | 164.207 | −5.72E−01 | 8.23E−02 | 1 | Unchanged Low |
| P55211 | 115.3899 | 9.62E−02 | −2.94E−02 | 1 | Unchanged Low |
| P21283 | 232.7446 | −1.25E+00 | −1.78E−03 | 1 | Unchanged Low |
| O43422 | 117.7416 | −1.19E−01 | −4.00E−01 | 1 | Unchanged Low |
| P78406 | 121.9098 | −2.76E−01 | −5.99E−01 | 1 | Unchanged Low |
| P43362 | 119.97 | 2.95E−01 | 4.92E−01 | 1 | Unchanged Low |
| Q9H693 | 129.9396 | −4.98E−01 | −8.33E−01 | 1 | Unchanged Low |
| Q9UJ83 | 120.5495 | 2.91E−01 | 4.99E−01 | 1 | Unchanged Low |
| Q05048 | 117.088 | −2.92E−02 | −2.36E−01 | 1 | Unchanged Low |
| P19784 | 122.6579 | 3.63E−01 | 6.78E−01 | 1 | Unchanged Low |
| Q9BUX8 | 155.6701 | −4.59E−01 | 8.56E−02 | 1 | Unchanged Low |
| Q9Y5P6 | 125.8828 | −4.50E−01 | −8.97E−01 | 1 | Unchanged Low |
| Q9Y3E1 | 122.172 | 3.30E−01 | 6.05E−01 | 1 | Unchanged Low |
| P40426 | 113.1427 | −9.01E−03 | −3.72E−01 | 1 | Unchanged Low |
| P11217 | 182.286 | −1.37E+00 | −1.52E+00 | 1 | Unchanged Low |
| Q16563 | 121.3173 | 3.23E−01 | 5.63E−01 | 1 | Unchanged Low |
| O95698 | 119.6732 | 2.57E−01 | 3.97E−01 | 1 | Unchanged Low |
| P19447 | 121.7269 | 3.56E−01 | 6.30E−01 | 1 | Unchanged Low |
| Q9UK39 | 185.8825 | −5.09E−01 | 5.89E−01 | 1 | Unchanged Low |
| Q15785 | 187.6861 | −1.28E+00 | −1.04E+00 | 1 | Unchanged Low |
| Q99571 | 316.7241 | −1.77E+00 | −2.96E−02 | 1 | Unchanged Low |
| Q9BTY2 | 115.258 | −1.09E−01 | −5.28E−01 | 1 | Unchanged Low |
| Q9P0P1 | 153.8845 | −2.72E−01 | 3.71E−01 | 1 | Unchanged Low |
| Q9Y324 | 116.2093 | −7.52E−02 | −4.13E−01 | 1 | Unchanged Low |
| P37286 | 128.7921 | 3.10E−01 | 7.38E−01 | 1 | Unchanged Low |
| Q00577 | 120.658 | 7.91E−02 | 8.84E−02 | 1 | Unchanged Low |
| O75152 | 121.2139 | 2.73E−01 | 4.61E−01 | 1 | Unchanged Low |
| P23610 | 130.3149 | −4.28E−01 | −6.73E−01 | 1 | Unchanged Low |
| O75436 | 116.2363 | 5.44E−02 | −1.30E−01 | 1 | Unchanged Low |
| Q9HA40 | 117.8494 | 2.29E−01 | 2.71E−01 | 1 | Unchanged Low |
| Q9NX62 | 120.7522 | 2.42E−01 | 3.88E−01 | 1 | Unchanged Low |
| O76062 | 212.8749 | −1.29E+00 | −4.89E−01 | 1 | Unchanged Low |
| Q16445 | 120.3177 | −2.95E−01 | −7.83E−01 | 1 | Unchanged Low |
| Q08462 | 125.3933 | 1.92E−01 | 4.39E−01 | 1 | Unchanged Low |
| P78560 | 128.1152 | 1.80E−01 | 4.97E−01 | 1 | Unchanged Low |
| P19634 | 113.6214 | 4.65E−02 | −2.66E−01 | 1 | Unchanged Low |
| P28370 | 114.9572 | 9.02E−02 | −1.17E−01 | 1 | Unchanged Low |
| Q92503 | 141.9225 | −3.02E−01 | −3.46E−03 | 1 | Unchanged Low |
| Q9UL02 | 117.4294 | 2.19E−01 | 2.25E−01 | 1 | Unchanged Low |
| Q9Y5T5 | 120.0007 | 7.16E−02 | 3.32E−02 | 1 | Unchanged Low |
| | 134.1729 | −3.14E−01 | −2.78E−01 | 1 | Unchanged Low |
| P15848 | 122.7499 | −2.79E−01 | −6.45E−01 | 1 | Unchanged Low |
| Q9UP66 | 120.4437 | 1.09E−01 | 1.17E−01 | 1 | Unchanged Low |
| O75160 | 120.8501 | 1.55E−02 | −5.53E−02 | 1 | Unchanged Low |
| P78385 | 119.6793 | 2.92E−01 | 4.24E−01 | 1 | Unchanged Low |
| O94967 | 124.6247 | 4.20E−01 | 7.82E−01 | 1 | Unchanged Low |
| O95719 | 117.7043 | 1.91E−01 | 1.71E−01 | 1 | Unchanged Low |
| P48147 | 127.6048 | −7.64E−02 | −7.45E−03 | 1 | Unchanged Low |
| P20231 | 119.9323 | 2.11E−01 | 2.81E−01 | 1 | Unchanged Low |
| O95299 | 119.8634 | 2.03E−01 | 2.60E−01 | 1 | Unchanged Low |
| O75033 | 122.4835 | 2.48E−02 | 9.32E−03 | 1 | Unchanged Low |
| P51668 | 120.3 | 3.08E−01 | 4.61E−01 | 1 | Unchanged Low |
| P28289 | 233.0271 | −1.30E+00 | −1.62E−01 | 1 | Unchanged Low |
| Q15631 | 149.5989 | −4.85E−01 | −1.88E−01 | 1 | Unchanged Low |
| P49868 | 113.6937 | 1.76E−02 | −3.63E−01 | 1 | Unchanged Low |
| P51959 | 114.0387 | 8.50E−02 | −1.99E−01 | 1 | Unchanged Low |
| Q9Y5V0 | 139.0584 | 1.19E−01 | 6.54E−01 | 1 | Unchanged Low |
| P56270 | 170.7774 | −1.00E+00 | −8.26E−01 | 1 | Unchanged Low |
| Q9HD23 | 127.1502 | −1.86E−01 | −2.72E−01 | 1 | Unchanged Low |
| P78358 | 159.681 | −8.14E−01 | −6.68E−01 | 1 | Unchanged Low |
| O00327 | 142.5298 | 6.87E−02 | 6.50E−01 | 1 | Unchanged Low |
| Q9H969 | 130.1172 | 4.75E−01 | 9.89E−01 | 1 | Unchanged Low |
| O15504 | 149.5876 | −6.90E−01 | −6.92E−01 | 1 | Unchanged Low |
| P35250 | 115.9922 | 1.59E−01 | 1.21E−02 | 1 | Unchanged Low |
| Q9BXC0 | 118.786 | 2.51E−01 | 2.87E−01 | 1 | Unchanged Low |
| O15232 | 115.8185 | 1.37E−01 | −4.27E−02 | 1 | Unchanged Low |
| O76049 | 198.6303 | −4.66E−01 | 8.00E−01 | 1 | Unchanged Low |
| Q14145 | 146.2645 | −6.39E−01 | −6.90E−01 | 1 | Unchanged Low |
| P20848 | 127.9292 | 3.50E−01 | 7.23E−01 | 1 | Unchanged Low |
| Q9P0N6 | 115.304 | 1.54E−01 | −3.53E−02 | 1 | Unchanged Low |
| P05188 | 125.869 | 4.94E−01 | 8.94E−01 | 1 | Unchanged Low |
| Q9UBJ4 | 117.695 | −7.21E−02 | −4.30E−01 | 1 | Unchanged Low |
| Q13876 | 125.9829 | −2.11E−01 | −4.06E−01 | 1 | Unchanged Low |
| Q9UBF2 | 116.849 | 1.81E−01 | 6.78E−02 | 1 | Unchanged Low |
| P43026 | 126.3658 | 2.93E−01 | 5.77E−01 | 1 | Unchanged Low |
| Q9HD20 | 123.6472 | 1.62E−01 | 2.67E−01 | 1 | Unchanged Low |
| Q96KK6 | 116.8445 | 6.05E−02 | −1.80E−01 | 1 | Unchanged Low |
| P40617 | 130.7449 | 1.06E−02 | 2.04E−01 | 1 | Unchanged Low |
| O75651 | 128.2583 | −7.77E−02 | −4.74E−02 | 1 | Unchanged Low |
| Q9Y2S1 | 131.4839 | −1.17E−01 | −2.10E−02 | 1 | Unchanged Low |
| Q13330 | 116.0949 | 2.53E−01 | 1.71E−01 | 1 | Unchanged Low |
| Q9Y389 | 114.7055 | 1.19E−01 | −1.55E−01 | 1 | Unchanged Low |
| O60513 | 115.0129 | 1.40E−01 | −1.00E−01 | 1 | Unchanged Low |
| O00212 | 341.5178 | −1.70E+00 | 2.70E−01 | 1 | Unchanged Low |
| Q9NXE1 | 122.4125 | 3.81E−01 | 5.97E−01 | 1 | Unchanged Low |
| O95405 | 154.2554 | 1.38E−01 | 9.72E−01 | 1 | Unchanged Low |
| P01112 | 119.6125 | −4.91E−02 | −3.23E−01 | 1 | Unchanged Low |
| O15120 | 209.3705 | −1.08E+00 | −1.49E−01 | 1 | Unchanged Low |
| P12645 | 116.6372 | 2.20E−01 | 1.18E−01 | 1 | Unchanged Low |
| P50579 | 121.7158 | −2.05E−01 | −6.04E−01 | 1 | Unchanged Low |
| O14908 | 176.6683 | −7.04E−01 | −3.26E−02 | 1 | Unchanged Low |
| Q9HCC6 | 140.2118 | 6.13E−01 | 1.38E+00 | 1 | Unchanged Low |
| Q15737 | 125.8246 | 1.62E−01 | 3.14E−01 | 1 | Unchanged Low |
| Q9Y519 | 119.1056 | 3.24E−01 | 3.87E−01 | 1 | Unchanged Low |
| Q9Y5J0 | 116.7986 | 9.99E−02 | −1.33E−01 | 1 | Unchanged Low |
| P51687 | 119.5637 | 2.59E−01 | 2.77E−01 | 1 | Unchanged Low |
| P51178 | 138.0904 | −4.43E−03 | 3.56E−01 | 1 | Unchanged Low |
| Q9H8J6 | 148.4766 | 5.05E−02 | 7.00E−01 | 1 | Unchanged Low |
| O95900 | 182.738 | −3.94E−01 | 6.31E−01 | 1 | Unchanged Low |
| Q9Y6N3 | 120.3564 | 3.37E−01 | 4.35E−01 | 1 | Unchanged Low |
| Q07869 | 119.4872 | 7.16E−02 | −9.89E−02 | 1 | Unchanged Low |
| Q9Y333 | 116.4501 | 2.11E−01 | 6.39E−02 | 1 | Unchanged Low |
| Q9NPD1 | 205.7432 | −7.07E−01 | 4.67E−01 | 1 | Unchanged Low |
| Q15928 | 138.6304 | 3.20E−01 | 8.98E−01 | 1 | Unchanged Low |
| O43826 | 155.5451 | −8.63E−01 | −1.05E+00 | 1 | Unchanged Low |
| Q9NWL0 | 120.1186 | 1.48E−01 | 7.25E−02 | 1 | Unchanged Low |
| P31629 | 151.1911 | 1.24E−01 | 8.64E−01 | 1 | Unchanged Low |
| O15530 | 168.488 | −5.58E−01 | 5.15E−02 | 1 | Unchanged Low |
| Q9Y2R5 | 122.8701 | 2.50E−01 | 3.52E−01 | 1 | Unchanged Low |
| Q15208 | 163.2891 | −3.28E−01 | 3.64E−01 | 1 | Unchanged Low |
| Q9UET6 | 116.4386 | 3.04E−01 | 2.30E−01 | 1 | Unchanged Low |
| Q9H5N0 | 119.1911 | 3.07E−01 | 3.30E−01 | 1 | Unchanged Low |
| Q9H019 | 126.5367 | 3.04E−01 | 5.53E−01 | 1 | Unchanged Low |
| Q9Y673 | 119.1797 | 3.38E−01 | 3.81E−01 | 1 | Unchanged Low |
| O75690 | 132.5659 | 6.31E−02 | 3.01E−01 | 1 | Unchanged Low |
| Q9NUP6 | 122.1684 | 4.03E−01 | 5.84E−01 | 1 | Unchanged Low |
| Q14296 | 114.1093 | 5.10E−02 | −3.98E−01 | 1 | Unchanged Low |
| Q9NW55 | 120.8058 | 4.23E−01 | 5.70E−01 | 1 | Unchanged Low |
| O43639 | 115.5457 | −1.40E−01 | −8.13E−01 | 1 | Unchanged Low |
| P17544 | 115.9726 | 2.27E−01 | 4.79E−02 | 1 | Unchanged Low |
| O75911 | 152.0313 | −2.99E−01 | 1.43E−01 | 1 | Unchanged Low |
| Q06430 | 120.315 | 4.27E−01 | 5.58E−01 | 1 | Unchanged Low |
| Q13769 | 123.5081 | 5.05E−02 | −2.60E−02 | 1 | Unchanged Low |
| Q9H864 | 122.387 | 3.82E−01 | 5.45E−01 | 1 | Unchanged Low |
| O95500 | 163.6478 | −5.24E−01 | −1.90E−02 | 1 | Unchanged Low |
| P14222 | 120.5243 | 3.62E−01 | 4.47E−01 | 1 | Unchanged Low |
| Q9Y4C8 | 126.2296 | 2.72E−01 | 4.67E−01 | 1 | Unchanged Low |
| Q9H3H5 | 143.2265 | −6.04E−01 | −8.39E−01 | 1 | Unchanged Low |
| Q13433 | 116.7563 | 1.12E−01 | −1.76E−01 | 1 | Unchanged Low |
| Q00059 | 123.3486 | 1.96E−01 | 2.32E−01 | 1 | Unchanged Low |
| Q15119 | 120.0132 | 3.96E−01 | 4.81E−01 | 1 | Unchanged Low |
| O43791 | 117.7111 | 1.98E−01 | 3.49E−02 | 1 | Unchanged Low |
| Q14206 | 170.2393 | 9.21E−02 | 1.14E+00 | 1 | Unchanged Low |
| Q92926 | 116.1179 | 1.50E−01 | −1.27E−01 | 1 | Unchanged Low |
| Q9P288 | 136.3262 | −2.20E−01 | −1.72E−01 | 1 | Unchanged Low |
| Q9NZU5 | 139.9549 | −3.60E−01 | −3.61E−01 | 1 | Unchanged Low |
| Q13569 | 127.6936 | 6.22E−01 | 1.05E+00 | 1 | Unchanged Low |
| P48167 | 125.3664 | 2.24E−01 | 3.39E−01 | 1 | Unchanged Low |
| Q9NQR1 | 124.9651 | −2.69E−01 | −7.35E−01 | 1 | Unchanged Low |
| O43405 | 117.417 | 3.14E−01 | 2.31E−01 | 1 | Unchanged Low |
| Q9P028 | 121.9053 | 3.01E−01 | 3.59E−01 | 1 | Unchanged Low |
| Q9Y6E8 | 115.6628 | 2.32E−01 | 5.37E−03 | 1 | Unchanged Low |
| Q9NV65 | 136.3658 | 4.75E−01 | 1.03E+00 | 1 | Unchanged Low |
| Q9H0R8 | 143.1503 | 7.20E−02 | 5.52E−01 | 1 | Unchanged Low |
| O95817 | 116.5745 | 2.61E−01 | 9.05E−02 | 1 | Unchanged Low |
| P36956 | 125.9988 | −1.26E−01 | −3.60E−01 | 1 | Unchanged Low |
| P08236 | 122.0379 | 5.60E−02 | −1.21E−01 | 1 | Unchanged Low |
| Q13425 | 119.2218 | 2.70E−01 | 1.96E−01 | 1 | Unchanged Low |
| Q14676 | 122.2375 | 4.63E−01 | 6.30E−01 | 1 | Unchanged Low |
| P16106 | 120.7804 | 3.94E−01 | 4.68E−01 | 1 | Unchanged Low |
| Q14773 | 122.427 | 3.39E−01 | 4.24E−01 | 1 | Unchanged Low |
| O14736 | 126.6152 | 4.48E−02 | 1.47E−02 | 1 | Unchanged Low |
| Q9Y297 | 141.0555 | −2.41E−01 | −1.01E−01 | 1 | Unchanged Low |
| Q13585 | 121.1757 | 7.99E−02 | −1.14E−01 | 1 | Unchanged Low |
| O60858 | 116.2529 | 2.27E−01 | −6.09E−03 | 1 | Unchanged Low |
| Q9Y2Z6 | 121.3443 | 4.22E−01 | 5.29E−01 | 1 | Unchanged Low |
| Q9UG66 | 273.7118 | −1.30E+00 | 2.32E−01 | 1 | Unchanged Low |
| Q9Y6G5 | 125.8408 | 2.61E−01 | 3.83E−01 | 1 | Unchanged Low |
| P98155 | 115.483 | 1.91E−01 | −1.19E−01 | 1 | Unchanged Low |
| Q9NP50 | 140.8474 | 4.50E−01 | 1.07E+00 | 1 | Unchanged Low |
| P28328 | 223.2274 | −1.31E+00 | −5.56E−01 | 1 | Unchanged Low |
| Q969T4 | 115.6428 | 1.42E−01 | −2.24E−01 | 1 | Unchanged Low |
| P19827 | 185.0551 | −8.88E−01 | −3.62E−01 | 1 | Unchanged Low |
| O15096 | 120.4316 | 1.43E−01 | −3.31E−02 | 1 | Unchanged Low |
| Q9Y5J6 | 119.4401 | 3.39E−01 | 3.06E−01 | 1 | Unchanged Low |
| Q92889 | 128.238 | −2.83E−01 | −6.91E−01 | 1 | Unchanged Low |
| P08697 | 134.8048 | −9.67E−02 | −2.29E−02 | 1 | Unchanged Low |
| Q9UBX1 | 120.4495 | 1.93E−01 | 6.08E−02 | 1 | Unchanged Low |
| Q9NPI7 | 180.4148 | −8.90E−01 | −4.93E−01 | 1 | Unchanged Low |
| P11586 | 125.7751 | −1.21E−01 | −4.11E−01 | 1 | Unchanged Low |
| O96010 | 125.1912 | 5.14E−01 | 7.67E−01 | 1 | Unchanged Low |
| Q13188 | 127.4091 | 1.45E−01 | 1.99E−01 | 1 | Unchanged Low |
| Q13845 | 121.3988 | −1.68E−01 | −7.24E−01 | 1 | Unchanged Low |
| Q9Y5W9 | 157.7019 | −1.54E−01 | 4.56E−01 | 1 | Unchanged Low |
| O60539 | 119.7412 | 3.58E−02 | −3.10E−01 | 1 | Unchanged Low |
| O60885 | 114.4111 | 1.78E−01 | −2.29E−01 | 1 | Unchanged Low |
| Q9H2B0 | 133.1825 | 8.60E−02 | 2.61E−01 | 1 | Unchanged Low |
| P51582 | 118.649 | 1.61E−01 | −9.03E−02 | 1 | Unchanged Low |
| O00585 | 171.5056 | −3.74E−01 | 3.53E−01 | 1 | Unchanged Low |
| Q15131 | 135.7223 | −3.73E−01 | −6.38E−01 | 1 | Unchanged Low |
| O75148 | 129.5265 | 5.97E−01 | 9.99E−01 | 1 | Unchanged Low |
| Q15691 | 157.4437 | −4.15E−01 | −4.51E−02 | 1 | Unchanged Low |
| Q15779 | 135.0227 | −1.18E−01 | −8.51E−02 | 1 | Unchanged Low |
| Q9UHD2 | 128.1679 | 6.62E−01 | 1.06E+00 | 1 | Unchanged Low |
| Q9NSS3 | 117.5518 | 2.77E−01 | 9.44E−02 | 1 | Unchanged Low |
| Q13797 | 124.1726 | −2.67E−01 | −8.84E−01 | 1 | Unchanged Low |
| P46531 | 126.8111 | 6.01E−01 | 9.30E−01 | 1 | Unchanged Low |
| O43262 | 134.2594 | 7.65E−01 | 1.35E+00 | 1 | Unchanged Low |
| Q9Y2H6 | 142.9858 | −3.89E−01 | −4.22E−01 | 1 | Unchanged Low |
| P25788 | 224.7239 | −9.86E−01 | 1.40E−01 | 1 | Unchanged Low |
| Q9BQS8 | 167.5571 | −3.34E−01 | 3.33E−01 | 1 | Unchanged Low |
| Q9Y396 | 125.1558 | −3.16E−01 | −9.85E−01 | 1 | Unchanged Low |
| Q9P0R7 | 158.5209 | 4.43E−02 | 7.84E−01 | 1 | Unchanged Low |
| O15269 | 120.3129 | 4.24E−01 | 4.46E−01 | 1 | Unchanged Low |
| Q9UFX0 | 217.1357 | −8.35E−01 | 2.98E−01 | 1 | Unchanged Low |
| Q9H663 | 119.1698 | 4.15E−01 | 3.87E−01 | 1 | Unchanged Low |
| O75410 | 162.703 | −7.75E−01 | −7.53E−01 | 1 | Unchanged Low |
| Q9Y5N5 | 121.2285 | 4.00E−01 | 4.24E−01 | 1 | Unchanged Low |
| Q9BQ72 | 117.4135 | 3.02E−01 | 1.09E−01 | 1 | Unchanged Low |
| Q9Y303 | 128.5162 | −2.27E−01 | −6.08E−01 | 1 | Unchanged Low |
| Q9P005 | 350.7027 | −1.38E+00 | 7.64E−01 | 1 | Unchanged Low |
| Q9Y3A1 | 198.1031 | −6.08E−01 | 3.88E−01 | 1 | Unchanged Low |
| Q15172 | 146.553 | −3.56E−01 | −2.69E−01 | 1 | Unchanged Low |
| Q9BV19 | 122.8739 | 4.48E−01 | 5.43E−01 | 1 | Unchanged Low |
| Q14534 | 119.574 | −2.39E−01 | −1.13E+00 | 1 | Unchanged Low |
| Q9BS43 | 281.8569 | −2.17E+00 | −2.29E+00 | 1 | Unchanged Low |
| P49788 | 121.3412 | 4.41E−01 | 4.73E−01 | 1 | Unchanged Low |
| Q9UP83 | 124.3058 | 3.96E−01 | 4.89E−01 | 1 | Unchanged Low |
| Q9Y2X0 | 115.5741 | 1.75E−01 | −2.56E−01 | 1 | Unchanged Low |
| Q9Y3E3 | 119.5355 | 4.78E−01 | 4.76E−01 | 1 | Unchanged Low |
| O95183 | 126.6473 | 5.34E−01 | 7.81E−01 | 1 | Unchanged Low |
| Q13242 | 143.0016 | −2.41E−01 | −1.48E−01 | 1 | Unchanged Low |
| Q9Y5R5 | 131.5571 | 3.90E−01 | 6.81E−01 | 1 | Unchanged Low |
| P04181 | 116.724 | 2.34E−01 | −8.99E−02 | 1 | Unchanged Low |
| Q14254 | 138.6301 | 2.25E−02 | 2.38E−01 | 1 | Unchanged Low |
| Q9Y6E6 | 121.0265 | 4.86E−01 | 5.31E−01 | 1 | Unchanged Low |
| Q9H237 | 179.3923 | −1.64E−01 | 7.98E−01 | 1 | Unchanged Low |
| Q92785 | 119.7533 | 1.61E−01 | −1.26E−01 | 1 | Unchanged Low |
| O95458 | 120.4814 | 3.89E−02 | −3.67E−01 | 1 | Unchanged Low |
| P20749 | 194.2507 | −9.95E−01 | −5.05E−01 | 1 | Unchanged Low |
| Q9H2N8 | 132.1507 | 3.45E−01 | 6.14E−01 | 1 | Unchanged Low |
| Q15020 | 132.2727 | 1.77E−01 | 3.26E−01 | 1 | Unchanged Low |
| P42025 | 156.1129 | −8.79E−01 | −1.41E+00 | 1 | Unchanged Low |
| P98173 | 120.727 | 2.31E−01 | 4.31E−02 | 1 | Unchanged Low |
| O95295 | 192.0882 | −1.29E+00 | −1.44E+00 | 1 | Unchanged Low |
| Q9NVA1 | 114.8529 | 2.48E−01 | −1.59E−01 | 1 | Unchanged Low |
| P35573 | 116.8065 | 2.46E−01 | −8.27E−02 | 1 | Unchanged Low |
| Q13126 | 190.4847 | −6.10E−01 | 2.17E−01 | 1 | Unchanged Low |
| P43034 | 121.4511 | 3.16E−01 | 2.22E−01 | 1 | Unchanged Low |
| Q01844 | 115.5021 | 2.57E−01 | −1.22E−01 | 1 | Unchanged Low |
| P27482 | 117.2725 | 2.14E−01 | −1.40E−01 | 1 | Unchanged Low |
| Q9NUW4 | 144.6673 | −7.51E−01 | −1.54E+00 | 1 | Unchanged Low |
| P78552 | 115.0749 | 1.70E−01 | −3.33E−01 | 1 | Unchanged Low |
| P51843 | 116.7757 | 3.46E−01 | 1.03E−01 | 1 | Unchanged Low |
| P15822 | 125.3978 | 6.78E−01 | 9.40E−01 | 1 | Unchanged Low |
| Q9BXY9 | 121.2561 | 5.53E−01 | 6.20E−01 | 1 | Unchanged Low |
| Q9ULB4 | 181.8847 | −1.04E+00 | −1.01E+00 | 1 | Unchanged Low |
| Q9NZZ7 | 204.594 | −6.87E−01 | 3.04E−01 | 1 | Unchanged Low |
| Q02086 | 122.3826 | 4.02E−01 | 3.90E−01 | 1 | Unchanged Low |
| Q9BUV9 | 118.2651 | 3.88E−01 | 2.22E−01 | 1 | Unchanged Low |
| Q9UHJ9 | 118.5627 | 4.07E−01 | 2.67E−01 | 1 | Unchanged Low |
| Q16401 | 132.2946 | −1.04E−01 | −2.63E−01 | 1 | Unchanged Low |
| Q9Y4K4 | 141.2069 | 1.37E−02 | 2.51E−01 | 1 | Unchanged Low |
| O60463 | 129.2479 | 3.73E−01 | 5.47E−01 | 1 | Unchanged Low |
| O95639 | 128.1539 | −4.54E−01 | −1.44E+00 | 1 | Unchanged Low |
| P50539 | 165.4016 | −3.93E−01 | 8.87E−02 | 1 | Unchanged Low |
| Q9UH62 | 140.9912 | 1.77E−01 | 5.32E−01 | 1 | Unchanged Low |
| O15525 | 116.1167 | 3.45E−01 | 5.31E−02 | 1 | Unchanged Low |
| Q9H8T0 | 160.2791 | 3.33E−01 | 1.18E+00 | 1 | Unchanged Low |
| Q9Y4X5 | 120.4976 | 4.29E−01 | 3.66E−01 | 1 | Unchanged Low |
| Q9H9E1 | 128.4841 | −3.65E−01 | −1.13E+00 | 1 | Unchanged Low |
| Q93063 | 139.4501 | 1.24E−01 | 3.82E−01 | 1 | Unchanged Low |
| Q9H992 | 124.4411 | 5.87E−01 | 7.37E−01 | 1 | Unchanged Low |
| O43237 | 181.4903 | −1.11E+00 | −1.28E+00 | 1 | Unchanged Low |
| Q99643 | 134.443 | −9.24E−02 | −1.95E−01 | 1 | Unchanged Low |
| P24386 | 119.0446 | 5.20E−01 | 4.56E−01 | 1 | Unchanged Low |
| Q9Y5F7 | 116.632 | 4.03E−01 | 1.58E−01 | 1 | Unchanged Low |
| P06756 | 184.6891 | −5.72E−01 | 1.33E−01 | 1 | Unchanged Low |
| Q9H5L7 | 152.0822 | −3.68E−01 | −2.34E−01 | 1 | Unchanged Low |
| O15517 | 144.7566 | 5.44E−01 | 1.17E+00 | 1 | Unchanged Low |
| Q9P0N5 | 123.7736 | 3.18E−01 | 2.53E−01 | 1 | Unchanged Low |
| Q9NWT2 | 115.8334 | 3.20E−01 | −4.05E−02 | 1 | Unchanged Low |
| Q9Y2A0 | 116.8334 | 8.19E−02 | −5.30E−01 | 1 | Unchanged Low |
| Q9NZ43 | 137.7883 | −6.58E−02 | −4.24E−02 | 1 | Unchanged Low |
| P27986 | 119.3503 | 2.77E−01 | 4.28E−03 | 1 | Unchanged Low |
| Q9UBP6 | 121.358 | 3.32E−01 | 1.85E−01 | 1 | Unchanged Low |
| Q9BWK5 | 119.8654 | 4.17E−01 | 2.88E−01 | 1 | Unchanged Low |
| P54802 | 135.7965 | −6.09E−01 | −1.64E+00 | 1 | Unchanged Low |
| O75963 | 143.3711 | 2.94E−01 | 7.40E−01 | 1 | Unchanged Low |
| Q9NXZ4 | 121.5448 | 1.21E−01 | −2.50E−01 | 1 | Unchanged Low |
| O60353 | 122.5147 | 6.62E−01 | 7.73E−01 | 1 | Unchanged Low |
| Q9NQS6 | 130.917 | 5.25E−01 | 7.90E−01 | 1 | Unchanged Low |
| O14776 | 122.1137 | 2.59E−01 | 4.90E−02 | 1 | Unchanged Low |
| Q9Y5A2 | 158.5684 | −1.83E−01 | 2.71E−01 | 1 | Unchanged Low |
| P49842 | 146.5953 | 2.52E−02 | 3.56E−01 | 1 | Unchanged Low |
| Q06546 | 120.885 | 6.10E−01 | 6.34E−01 | 1 | Unchanged Low |
| Q9Y2E7 | 116.739 | 3.06E−01 | −7.00E−02 | 1 | Unchanged Low |
| Q9UM11 | 134.12 | 7.50E−01 | 1.20E+00 | 1 | Unchanged Low |
| Q99447 | 129.39 | −3.62E−01 | −1.18E+00 | 1 | Unchanged Low |
| Q9H1R3 | 119.5324 | 5.40E−01 | 4.63E−01 | 1 | Unchanged Low |
| P28358 | 115.6766 | 3.21E−01 | −9.71E−02 | 1 | Unchanged Low |
| O76070 | 134.744 | 8.30E−02 | 1.18E−01 | 1 | Unchanged Low |
| Q9Y5A7 | 149.951 | 1.95E−01 | 7.08E−01 | 1 | Unchanged Low |
| Q9NWY7 | 116.6328 | 3.94E−01 | 8.20E−02 | 1 | Unchanged Low |
| O43715 | 127.9562 | −1.23E−02 | −3.20E−01 | 1 | Unchanged Low |
| Q9BY75 | 127.3323 | 2.03E−01 | 9.81E−02 | 1 | Unchanged Low |
| O43431 | 144.6131 | 1.11E−01 | 4.34E−01 | 1 | Unchanged Low |
| O43567 | 133.3964 | 3.06E−01 | 4.72E−01 | 1 | Unchanged Low |
| Q9Y2S7 | 117.8414 | 2.73E−01 | −1.26E−01 | 1 | Unchanged Low |
| O14582 | 135.7349 | 1.66E−02 | 1.46E−03 | 1 | Unchanged Low |
| O75056 | 139.562 | 1.88E−01 | 4.31E−01 | 1 | Unchanged Low |
| Q9P2Y2 | 123.2251 | 6.09E−01 | 6.75E−01 | 1 | Unchanged Low |
| Q16595 | 135.5917 | −2.85E−02 | −9.82E−02 | 1 | Unchanged Low |
| Q14807 | 115.2048 | 2.58E−01 | −2.81E−01 | 1 | Unchanged Low |
| Q9BZL4 | 123.5234 | 3.10E−01 | 1.54E−01 | 1 | Unchanged Low |
| P30876 | 200.7168 | −9.54E−01 | −4.12E−01 | 1 | Unchanged Low |
| Q9UGP6 | 122.0845 | 5.35E−01 | 5.02E−01 | 1 | Unchanged Low |
| P52655 | 129.1154 | 1.77E−01 | 8.08E−02 | 1 | Unchanged Low |
| Q9HB07 | 125.5248 | 1.81E−01 | −4.11E−02 | 1 | Unchanged Low |
| Q92940 | 258.3032 | −1.59E+00 | −8.95E−01 | 1 | Unchanged Low |
| Q9NWN1 | 124.9938 | 2.25E−01 | 2.24E−02 | 1 | Unchanged Low |
| O43664 | 116.0604 | 3.49E−01 | −7.90E−02 | 1 | Unchanged Low |
| O14647 | 132.5407 | 5.19E−01 | 7.68E−01 | 1 | Unchanged Low |
| O75333 | 130.9257 | 1.29E−01 | 3.38E−02 | 1 | Unchanged Low |
| Q16539 | 168.4312 | −1.10E−01 | 5.57E−01 | 1 | Unchanged Low |
| Q9NQG5 | 122.4598 | 6.67E−01 | 7.18E−01 | 1 | Unchanged Low |
| Q15149 | 115.8848 | 1.35E−01 | −5.84E−01 | 1 | Unchanged Low |
| Q9NVZ7 | 159.6342 | −2.67E−01 | 7.06E−02 | 1 | Unchanged Low |
| Q15800 | 127.0641 | −3.15E−01 | −1.27E+00 | 1 | Unchanged Low |
| Q9H0B6 | 121.3672 | 5.17E−01 | 4.22E−01 | 1 | Unchanged Low |
| P09001 | 120.1502 | 5.53E−01 | 4.44E−01 | 1 | Unchanged Low |
| O00301 | 117.5428 | 1.34E−01 | −5.14E−01 | 1 | Unchanged Low |
| P55786 | 170.346 | −9.84E−01 | −1.47E+00 | 1 | Unchanged Low |
| Q9Y5Y9 | 134.9928 | 1.20E−01 | 1.28E−01 | 1 | Unchanged Low |
| P19235 | 138.1099 | −5.20E−01 | −1.36E+00 | 1 | Unchanged Low |
| O15228 | 183.349 | −7.01E−01 | −2.72E−01 | 1 | Unchanged Low |
| Q9UK97 | 131.097 | −1.25E−01 | −5.43E−01 | 1 | Unchanged Low |
| P50990 | 197.1593 | −9.88E−01 | −6.28E−01 | 1 | Unchanged Low |
| Q9NZM1 | 186.4122 | −6.59E−01 | −1.17E−01 | 1 | Unchanged Low |
| Q92924 | 117.704 | 7.37E−02 | −6.73E−01 | 1 | Unchanged Low |
| O75845 | 131.1196 | 4.45E−01 | 5.83E−01 | 1 | Unchanged Low |
| Q9NQ88 | 125.605 | 7.91E−01 | 9.75E−01 | 1 | Unchanged Low |
| Q16515 | 116.6136 | 4.45E−01 | 9.06E−02 | 1 | Unchanged Low |
| P49759 | 124.2553 | 6.90E−01 | 7.73E−01 | 1 | Unchanged Low |
| P33552 | 114.8881 | 3.45E−01 | −1.91E−01 | 1 | Unchanged Low |
| O75808 | 117.8898 | 2.67E−01 | −2.26E−01 | 1 | Unchanged Low |
| Q9NXR1 | 119.5057 | 5.33E−01 | 3.56E−01 | 1 | Unchanged Low |
| O75381 | 241.8793 | −1.77E+00 | −2.07E+00 | 1 | Unchanged Low |
| O60930 | 139.1464 | −2.65E−01 | −5.95E−01 | 1 | Unchanged Low |
| O14653 | 114.9152 | 2.66E−01 | −3.68E−01 | 1 | Unchanged Low |
| Q16540 | 156.1365 | −5.87E−01 | −8.04E−01 | 1 | Unchanged Low |
| O14658 | 154.6374 | −1.83E−01 | 7.29E−02 | 1 | Unchanged Low |
| Q15814 | 118.2869 | 4.34E−01 | 1.14E−01 | 1 | Unchanged Low |
| Q9Y3C9 | 128.8568 | 7.42E−01 | 9.69E−01 | 1 | Unchanged Low |
| P18827 | 115.5895 | 2.88E−01 | −3.03E−01 | 1 | Unchanged Low |
| P04150 | 116.6017 | 3.89E−01 | −4.49E−02 | 1 | Unchanged Low |
| Q9Y301 | 116.3594 | 3.71E−01 | −9.27E−02 | 1 | Unchanged Low |
| O00626 | 119.8588 | 3.01E−01 | −9.45E−02 | 1 | Unchanged Low |
| | 127.048 | 3.72E−01 | 2.98E−01 | 1 | Unchanged Low |
| Q9Y311 | 125.8788 | −2.18E−01 | −1.12E+00 | 1 | Unchanged Low |
| Q15633 | 144.9588 | −2.75E−01 | −4.33E−01 | 1 | Unchanged Low |
| P02278 | 152.2309 | 4.82E−01 | 1.11E+00 | 1 | Unchanged Low |
| P53672 | 142.8494 | 4.64E−01 | 8.75E−01 | 1 | Unchanged Low |
| P09417 | 142.5395 | 1.48E−01 | 3.43E−01 | 1 | Unchanged Low |
| Q9HAY2 | 120.9341 | 5.48E−01 | 3.97E−01 | 1 | Unchanged Low |
| P30273 | 133.3475 | 6.67E−01 | 9.51E−01 | 1 | Unchanged Low |
| Q92949 | 185.89 | −6.34E−01 | −5.87E−01 | 1 | Unchanged Low |
| Q9GZT9 | 117.5626 | 3.18E−01 | −1.85E−01 | 1 | Unchanged Low |
| P35658 | 139.8811 | −1.07E−02 | −4.96E−02 | 1 | Unchanged Low |
| Q9HB40 | 115.3964 | 4.12E−01 | −9.32E−02 | 1 | Unchanged Low |
| O14686 | 138.5398 | 2.60E−01 | 4.11E−01 | 1 | Unchanged Low |
| Q92911 | 176.594 | −9.05E−01 | −1.08E+00 | 1 | Unchanged Low |
| Q9Y5E4 | 127.9379 | 5.70E−01 | 6.34E−01 | 1 | Unchanged Low |
| Q13057 | 151.0906 | −4.04E−01 | −5.78E−01 | 1 | Unchanged Low |
| Q14863 | 115.7954 | 4.53E−01 | −4.97E−03 | 1 | Unchanged Low |
| P36610 | 129.0874 | 3.60E−01 | 2.98E−01 | 1 | Unchanged Low |
| O60240 | 438.2653 | −2.99E+00 | −4.43E+00 | 1 | Unchanged Low |
| Q9H9B4 | 120.8973 | 6.87E−01 | 5.96E−01 | 1 | Unchanged Low |
| O00584 | 172.0542 | 1.85E−01 | 1.00E+00 | 1 | Unchanged Low |
| Q9H658 | 171.1973 | −1.58E−01 | 4.32E−01 | 1 | Unchanged Low |
| P18858 | 120.8853 | 6.20E−01 | 4.76E−01 | 1 | Unchanged Low |
| Q02877 | 295.8854 | −1.66E+00 | −5.99E−01 | 1 | Unchanged Low |
| P15586 | 174.2861 | −8.81E−01 | −1.13E+00 | 1 | Unchanged Low |
| P42696 | 161.4649 | 3.56E−01 | 1.06E+00 | 1 | Unchanged Low |
| O75710 | 116.2449 | 4.32E−01 | −5.47E−02 | 1 | Unchanged Low |
| Q9NPI8 | 183.3737 | −4.48E−01 | 1.45E−01 | 1 | Unchanged Low |
| Q9HAR6 | 152.8734 | 2.56E−01 | 7.32E−01 | 1 | Unchanged Low |
| P15018 | 235.0602 | −8.35E−01 | 2.84E−01 | 1 | Unchanged Low |
| Q13546 | 137.7012 | 2.63E−02 | −9.15E−02 | 1 | Unchanged Low |
| P24298 | 149.9584 | −5.64E−01 | −1.12E+00 | 1 | Unchanged Low |
| Q14129 | 131.8973 | 3.45E−01 | 3.32E−01 | 1 | Unchanged Low |
| Q9BQA2 | 164.7437 | −2.12E−01 | 1.80E−01 | 1 | Unchanged Low |
| Q9UJX9 | 120.9085 | 2.27E−01 | −3.06E−01 | 1 | Unchanged Low |
| Q99590 | 190.7413 | −6.97E−01 | −2.18E−01 | 1 | Unchanged Low |
| O14798 | 166.073 | −1.68E−03 | 5.78E−01 | 1 | Unchanged Low |
| P32238 | 265.9537 | −1.77E+00 | −1.54E+00 | 1 | Unchanged Low |
| Q9UQ90 | 129.9052 | 4.94E−01 | 5.26E−01 | 1 | Unchanged Low |
| P82980 | 115.9454 | 3.60E−01 | −2.44E−01 | 1 | Unchanged Low |
| P49716 | 128.4871 | −2.44E−01 | −1.20E+00 | 1 | Unchanged Low |
| P20062 | 136.6279 | 1.85E−01 | 1.71E−01 | 1 | Unchanged Low |
| P21580 | 245.0939 | −9.49E−01 | 1.99E−01 | 1 | Unchanged Low |
| Q99774 | 170.7835 | −4.52E−01 | −1.76E−01 | 1 | Unchanged Low |
| Q9P0T1 | 116.1881 | 4.56E−01 | −5.00E−02 | 1 | Unchanged Low |
| P06737 | 122.6343 | 7.51E−01 | 7.12E−01 | 1 | Unchanged Low |
| O15063 | 145.705 | −6.52E−02 | −5.91E−02 | 1 | Unchanged Low |
| P02743 | 115.3343 | 4.08E−01 | −1.90E−01 | 1 | Unchanged Low |
| Q9NVT0 | 129.6057 | 7.10E−02 | −3.23E−01 | 1 | Unchanged Low |
| Q9Y4P3 | 119.7013 | 3.56E−01 | −1.14E−01 | 1 | Unchanged Low |
| Q13886 | 132.2194 | −1.96E−01 | −8.90E−01 | 1 | Unchanged Low |
| Q9NVP8 | 117.1232 | 4.96E−01 | 5.83E−02 | 1 | Unchanged Low |
| P08240 | 144.5673 | −1.40E−01 | −2.66E−01 | 1 | Unchanged Low |
| P49459 | 137.6267 | 5.43E−01 | 7.89E−01 | 1 | Unchanged Low |
| Q9NVAB | 127.9498 | 3.52E−01 | 1.78E−01 | 1 | Unchanged Low |
| P25325 | 235.1972 | −1.00E+00 | −7.45E−02 | 1 | Unchanged Low |
| Q9UHI6 | 162.7208 | 8.43E−02 | 6.21E−01 | 1 | Unchanged Low |
| P52298 | 157.996 | 5.06E−02 | 4.61E−01 | 1 | Unchanged Low |
| Q9NQ30 | 123.2921 | 4.67E−01 | 2.24E−01 | 1 | Unchanged Low |
| O00541 | 176.2086 | −3.24E−01 | 1.78E−01 | 1 | Unchanged Low |
| Q9H2V9 | 162.3327 | −2.68E−01 | −3.56E−02 | 1 | Unchanged Low |
| Q9NNX1 | 155.2699 | −2.90E−02 | 2.49E−01 | 1 | Unchanged Low |
| Q13508 | 118.095 | 3.98E−01 | −1.19E−01 | 1 | Unchanged Low |
| Q9NR30 | 128.2929 | 3.85E−01 | 2.36E−01 | 1 | Unchanged Low |
| Q14320 | 141.6929 | 4.93E−01 | 7.99E−01 | 1 | Unchanged Low |
| Q04726 | 156.9544 | −5.54E−01 | −8.86E−01 | 1 | Unchanged Low |
| P20061 | 115.787 | 3.00E−01 | −4.56E−01 | 1 | Unchanged Low |
| Q9Y2Y1 | 147.7222 | −8.89E−02 | −8.58E−02 | 1 | Unchanged Low |
| P35754 | 145.4287 | −8.79E−02 | −1.59E−01 | 1 | Unchanged Low |
| Q02978 | 116.628 | 4.38E−01 | −1.22E−01 | 1 | Unchanged Low |
| O95792 | 140.5872 | 1.48E−01 | 1.63E−01 | 1 | Unchanged Low |
| Q13232 | 130.592 | 6.74E−01 | 7.82E−01 | 1 | Unchanged Low |
| Q9NVH2 | 117.932 | 3.01E−01 | −3.68E−01 | 1 | Unchanged Low |
| Q15561 | 183.8604 | −6.89E−01 | −4.46E−01 | 1 | Unchanged Low |
| Q9UPN9 | 130.2668 | 3.90E−01 | 2.78E−01 | 1 | Unchanged Low |
| Q14353 | 119.1987 | 4.14E−01 | −8.24E−02 | 1 | Unchanged Low |
| Q9NZM5 | 139.1802 | −4.40E−02 | −3.01E−01 | 1 | Unchanged Low |
| O43255 | 120.4593 | 3.46E−01 | −1.81E−01 | 1 | Unchanged Low |
| Q14331 | 161.7807 | −1.72E−01 | 9.77E−02 | 1 | Unchanged Low |
| P18847 | 142.5299 | 1.92E−01 | 2.75E−01 | 1 | Unchanged Low |
| O60258 | 176.0472 | −1.33E−01 | 4.79E−01 | 1 | Unchanged Low |
| P47929 | 129.8767 | −5.31E−02 | −7.04E−01 | 1 | Unchanged Low |
| P35227 | 135.0089 | 4.30E−01 | 4.80E−01 | 1 | Unchanged Low |
| Q15599 | 131.8082 | −2.11E−01 | −1.07E+00 | 1 | Unchanged Low |
| Q9UKJ5 | 178.8637 | −3.37E−01 | 1.60E−01 | 1 | Unchanged Low |
| Q9HCB9 | 122.1779 | 6.34E−01 | 4.22E−01 | 1 | Unchanged Low |
| Q9Y3A0 | 147.7783 | 2.95E−01 | 5.80E−01 | 1 | Unchanged Low |
| P49815 | 138.4231 | 4.64E−01 | 6.20E−01 | 1 | Unchanged Low |
| P25116 | 123.8585 | 8.27E−01 | 7.92E−01 | 1 | Unchanged Low |
| O75829 | 128.9495 | 2.67E−01 | −3.81E−02 | 1 | Unchanged Low |
| Q9NYI1 | 122.8588 | 3.91E−01 | −2.14E−02 | 1 | Unchanged Low |
| P24347 | 126.9018 | 5.88E−01 | 4.84E−01 | 1 | Unchanged Low |
| O43189 | 120.038 | 3.88E−01 | −1.46E−01 | 1 | Unchanged Low |
| Q9P029 | 168.9188 | −4.56E−01 | −3.41E−01 | 1 | Unchanged Low |
| Q9NZP9 | 136.3041 | 4.81E−01 | 5.78E−01 | 1 | Unchanged Low |
| Q9UIA3 | 153.0756 | −1.74E−01 | −1.74E−01 | 1 | Unchanged Low |
| Q99734 | 210.2011 | −5.84E−01 | 2.56E−01 | 1 | Unchanged Low |
| O00411 | 124.1084 | 2.87E−01 | −2.02E−01 | 1 | Unchanged Low |
| P28347 | 183.776 | −6.46E−01 | −4.04E−01 | 1 | Unchanged Low |
| Q13619 | 172.6621 | −5.72E−02 | 5.07E−01 | 1 | Unchanged Low |
| O94929 | 118.4779 | 4.37E−01 | −1.31E−01 | 1 | Unchanged Low |
| Q9P026 | 149.0331 | 3.51E−01 | 6.74E−01 | 1 | Unchanged Low |
| P28067 | 125.4983 | 1.96E−01 | −3.58E−01 | 1 | Unchanged Low |
| Q14126 | 313.3393 | −1.53E+00 | −2.23E−01 | 1 | Unchanged Low |
| Q14267 | 124.7021 | 2.06E−01 | −3.70E−01 | 1 | Unchanged Low |
| P55197 | 164.3164 | −1.24E−01 | 2.02E−01 | 1 | Unchanged Low |
| Q9NP87 | 116.4497 | 3.57E−01 | −4.10E−01 | 1 | Unchanged Low |
| P04155 | 180.4791 | −8.07E−01 | −9.25E−01 | 1 | Unchanged Low |
| Q9C069 | 207.9658 | 1.49E+00 | 3.01E+00 | 1 | Unchanged Low |
| P12270 | 129.4065 | 5.00E−01 | 3.81E−01 | 1 | Unchanged Low |
| Q9UIG6 | 230.2685 | −5.62E−01 | 5.72E−01 | 1 | Unchanged Low |
| O15453 | 118.2765 | 5.78E−01 | 1.22E−01 | 1 | Unchanged Low |
| Q9UNW8 | 244.7569 | −6.93E−01 | 5.36E−01 | 1 | Unchanged Low |
| P06746 | 171.8166 | 5.50E−02 | 6.58E−01 | 1 | Unchanged Low |
| O15414 | 159.7305 | 1.70E−01 | 6.00E−01 | 1 | Unchanged Low |
| O15259 | 124.437 | 3.67E−01 | −6.59E−02 | 1 | Unchanged Low |
| Q9NRA8 | 144.3142 | 4.81E−01 | 7.45E−01 | 1 | Unchanged Low |
| Q9NP77 | 165.0639 | −2.05E−01 | 3.60E−02 | 1 | Unchanged Low |
| Q06587 | 131.6381 | 3.18E−01 | 9.12E−02 | 1 | Unchanged Low |
| Q9Y5Z5 | 175.2357 | −2.84E−01 | 1.13E−01 | 1 | Unchanged Low |
| O15327 | 154.5722 | −3.04E−01 | −4.85E−01 | 1 | Unchanged Low |
| P49646 | 136.7074 | 4.83E−02 | −2.95E−01 | 1 | Unchanged Low |
| Q99809 | 124.7728 | 1.53E−01 | −5.53E−01 | 1 | Unchanged Low |
| Q9UBM1 | 136.3565 | 1.06E−01 | −1.88E−01 | 1 | Unchanged Low |
| P16403 | 377.9287 | −1.52E+00 | 3.84E−01 | 1 | Unchanged Low |
| O75503 | 124.5021 | 1.33E−03 | −9.83E−01 | 1 | Unchanged Low |
| P15814 | 123.9169 | 9.19E−01 | 8.64E−01 | 1 | Unchanged Low |
| Q03164 | 129.4559 | −7.26E−02 | −9.39E−01 | 1 | Unchanged Low |
| Q13624 | 138.6386 | 1.32E−01 | −6.85E−02 | 1 | Unchanged Low |
| Q14118 | 118.1383 | 5.00E−01 | −9.59E−02 | 1 | Unchanged Low |
| Q13286 | 230.6187 | −6.85E−01 | 3.27E−01 | 1 | Unchanged Low |
| Q9Y5Q0 | 127.2281 | 3.55E−01 | −2.72E−02 | 1 | Unchanged Low |
| Q9P0R1 | 120.8481 | 5.95E−01 | 1.96E−01 | 1 | Unchanged Low |
| P41182 | 117.2275 | 3.70E−01 | −4.34E−01 | 1 | Unchanged Low |
| O75497 | 153.0143 | 4.04E−02 | 1.64E−01 | 1 | Unchanged Low |
| Q14185 | 118.9819 | 6.54E−01 | 2.27E−01 | 1 | Unchanged Low |
| O95163 | 157.6357 | −4.36E−01 | −7.60E−01 | 1 | Unchanged Low |
| Q14508 | 151.3745 | 5.49E−01 | 9.73E−01 | 1 | Unchanged Low |
| P25942 | 166.2403 | 4.47E−01 | 1.12E+00 | 1 | Unchanged Low |
| Q9Y6E0 | 194.1145 | −4.60E−01 | 1.25E−01 | 1 | Unchanged Low |
| Q9UQF2 | 187.4786 | −6.13E−01 | −3.36E−01 | 1 | Unchanged Low |
| Q9Y285 | 158.2291 | −2.58E−01 | −3.14E−01 | 1 | Unchanged Low |
| Q04762 | 153.4581 | 1.65E−01 | 3.90E−01 | 1 | Unchanged Low |
| Q9Y6J9 | 128.6968 | 9.55E−01 | 1.03E+00 | 1 | Unchanged Low |
| O43913 | 155.6272 | 2.48E−02 | 1.88E−01 | 1 | Unchanged Low |
| P25208 | 131.7807 | 4.99E−01 | 3.77E−01 | 1 | Unchanged Low |
| Q9H9X4 | 173.1977 | −3.32E−01 | −7.94E−02 | 1 | Unchanged Low |
| O95297 | 133.0095 | 2.42E−01 | −7.72E−02 | 1 | Unchanged Low |
| Q9UPG8 | 150.4388 | 1.62E−01 | 2.95E−01 | 1 | Unchanged Low |
| P16333 | 246.4108 | −6.52E−01 | 5.66E−01 | 1 | Unchanged Low |
| Q14108 | 148.3416 | −3.98E−02 | −1.61E−01 | 1 | Unchanged Low |
| Q9UFF9 | 174.1041 | −1.09E−01 | 3.55E−01 | 1 | Unchanged Low |
| O15242 | 186.2788 | −4.75E−01 | −8.72E−02 | 1 | Unchanged Low |
| Q13442 | 165.0545 | −4.70E−02 | 2.67E−01 | 1 | Unchanged Low |
| Q99755 | 136.4804 | 4.32E−01 | 3.78E−01 | 1 | Unchanged Low |
| Q9NZV1 | 121.0073 | 4.27E−01 | −1.85E−01 | 1 | Unchanged Low |
| Q05195 | 180.6838 | 1.47E−01 | 8.89E−01 | 1 | Unchanged Low |
| O95671 | 116.6189 | 6.06E−01 | −1.74E−02 | 1 | Unchanged Low |
| Q9Y658 | 120.0494 | 4.27E−01 | −2.46E−01 | 1 | Unchanged Low |
| Q9BTU6 | 142.5367 | 6.43E−01 | 8.74E−01 | 1 | Unchanged Low |
| Q9UPN6 | 130.9048 | 2.52E−01 | −1.76E−01 | 1 | Unchanged Low |
| O00170 | 310.1793 | −1.87E+00 | −1.31E+00 | 1 | Unchanged Low |
| Q9UJW6 | 186.4423 | −5.44E−01 | −2.60E−01 | 1 | Unchanged Low |
| Q9HCT0 | 145.1536 | −5.16E−02 | −3.30E−01 | 1 | Unchanged Low |
| Q15459 | 153.5048 | −1.01E−01 | −1.76E−01 | 1 | Unchanged Low |
| O95400 | 194.1745 | −3.34E−01 | 3.07E−01 | 1 | Unchanged Low |
| P07954 | 138.7259 | 3.31E−01 | 2.31E−01 | 1 | Unchanged Low |
| Q9H6Z5 | 135.2933 | 5.94E−01 | 5.88E−01 | 1 | Unchanged Low |
| Q9HCN8 | 139.1775 | 1.63E−01 | −9.27E−02 | 1 | Unchanged Low |
| Q92890 | 176.6159 | −4.00E−02 | 4.82E−01 | 1 | Unchanged Low |
| O43324 | 138.6164 | 2.17E−02 | −4.25E−01 | 1 | Unchanged Low |
| Q9NVU8 | 139.1241 | 7.28E−02 | −2.90E−01 | 1 | Unchanged Low |
| Q9NQW6 | 117.4953 | 4.26E−01 | −4.11E−01 | 1 | Unchanged Low |
| Q9NWH4 | 144.3461 | −1.80E−01 | −7.03E−01 | 1 | Unchanged Low |
| Q9UBV8 | 148.3188 | 4.84E−02 | −4.15E−02 | 1 | Unchanged Low |
| P15328 | 142.7655 | 5.77E−03 | −3.14E−01 | 1 | Unchanged Low |
| Q9Y583 | 157.8847 | 9.38E−02 | 2.99E−01 | 1 | Unchanged Low |
| Q07326 | 126.3257 | 2.09E−01 | −5.12E−01 | 1 | Unchanged Low |
| O75396 | 150.1024 | 9.19E−02 | 8.88E−02 | 1 | Unchanged Low |
| Q9Y2Z8 | 126.8764 | 3.88E−01 | −9.52E−02 | 1 | Unchanged Low |
| Q9H3P7 | 220.9899 | −7.10E−01 | 3.73E−02 | 1 | Unchanged Low |
| O95678 | 229.3684 | −1.14E+00 | −8.00E−01 | 1 | Unchanged Low |
| P17026 | 131.6407 | 1.08E−02 | −7.90E−01 | 1 | Unchanged Low |
| Q9Y3N9 | 139.4653 | 2.75E−01 | 1.09E−01 | 1 | Unchanged Low |
| Q9BYT8 | 144.2719 | 8.41E−01 | 1.16E+00 | 1 | Unchanged Low |
| Q9UHQ7 | 136.5651 | 3.69E−01 | 1.95E−01 | 1 | Unchanged Low |
| O60921 | 169.0172 | 5.29E−02 | 4.60E−01 | 1 | Unchanged Low |
| O00400 | 158.3654 | −3.53E−01 | −6.68E−01 | 1 | Unchanged Low |
| Q9Y690 | 222.1522 | −7.15E−01 | 3.23E−02 | 1 | Unchanged Low |
| Q9NZL9 | 127.4733 | 2.65E−01 | −3.65E−01 | 1 | Unchanged Low |
| O60563 | 168.3854 | −2.19E−01 | −6.81E−02 | 1 | Unchanged Low |
| P20591 | 495.4752 | −1.57E+00 | 9.27E−01 | 1 | Unchanged Low |
| Q9BQC6 | 139.1028 | −2.89E−01 | −1.34E+00 | 1 | Unchanged Low |
| P28838 | 315.6553 | −1.41E+00 | −9.33E−02 | 1 | Unchanged Low |
| Q9NZE5 | 153.3881 | 1.92E−01 | 3.36E−01 | 1 | Unchanged Low |
| Q9NX47 | 263.9291 | −9.55E−01 | 1.75E−01 | 1 | Unchanged Low |
| O60612 | 179.421 | 4.43E−01 | 1.25E+00 | 1 | Unchanged Low |
| O00559 | 160.9495 | −2.31E−01 | −3.09E−01 | 1 | Unchanged Low |
| Q9NUU9 | 172.9723 | 1.40E−01 | 6.73E−01 | 1 | Unchanged Low |
| P22059 | 127.2507 | 2.16E−01 | −5.10E−01 | 1 | Unchanged Low |
| O00408 | 394.9107 | −2.47E+00 | −2.34E+00 | 1 | Unchanged Low |
| O94935 | 142.9113 | 8.58E−02 | −1.94E−01 | 1 | Unchanged Low |
| P41134 | 293.8674 | −1.49E+00 | −5.70E−01 | 1 | Unchanged Low |
| P24407 | 189.5598 | −5.30E−01 | −2.43E−01 | 1 | Unchanged Low |
| O00232 | 139.0276 | 3.42E−01 | 1.85E−01 | 1 | Unchanged Low |
| Q01432 | 127.7664 | 4.83E−01 | 7.10E−02 | 1 | Unchanged Low |
| Q13525 | 140.5051 | 1.92E−01 | −6.63E−02 | 1 | Unchanged Low |
| Q16611 | 210.0516 | −8.50E−01 | −5.30E−01 | 1 | Unchanged Low |
| Q9NPA3 | 117.8671 | 4.67E−01 | −3.96E−01 | 1 | Unchanged Low |
| Q9GZM8 | 141.4354 | 1.75E−01 | −7.41E−02 | 1 | Unchanged Low |
| P42702 | 120.0363 | 7.51E−01 | 2.77E−01 | 1 | Unchanged Low |
| Q05940 | 227.6447 | −1.27E+00 | −1.29E+00 | 1 | Unchanged Low |
| Q9Y3Q5 | 127.0621 | 1.04E+00 | 9.77E−01 | 1 | Unchanged Low |
| Q15756 | 128.583 | 6.33E−02 | −8.81E−01 | 1 | Unchanged Low |
| O75386 | 122.3782 | 2.76E−01 | −6.39E−01 | 1 | Unchanged Low |
| P50750 | 213.7777 | −7.97E−01 | −3.35E−01 | 1 | Unchanged Low |
| Q9NWY6 | 125.9903 | 7.65E−01 | 5.01E−01 | 1 | Unchanged Low |
| P50391 | 134.9143 | 1.94E−01 | −2.85E−01 | 1 | Unchanged Low |
| Q9NX07 | 233.9363 | −7.26E−01 | 1.52E−01 | 1 | Unchanged Low |
| O75354 | 267.133 | −1.30E+00 | −5.81E−01 | 1 | Unchanged Low |
| Q9H832 | 212.3427 | −1.44E−01 | 8.22E−01 | 1 | Unchanged Low |
| Q9UBW8 | 123.4686 | 6.11E−01 | 1.05E−01 | 1 | Unchanged Low |
| P14136 | 117.3353 | 5.40E−01 | −3.21E−01 | 1 | Unchanged Low |
| Q9NYF3 | 124.4392 | 3.76E−01 | −3.64E−01 | 1 | Unchanged Low |
| Q15056 | 161.3602 | −3.61E−01 | −7.09E−01 | 1 | Unchanged Low |
| O15534 | 179.3665 | −5.89E−01 | −7.28E−01 | 1 | Unchanged Low |
| Q9Y399 | 175.5893 | −5.44E−01 | −7.25E−01 | 1 | Unchanged Low |
| O14972 | 218.3678 | −5.45E−01 | 2.16E−01 | 1 | Unchanged Low |
| Q14113 | 126.0667 | 3.38E−01 | −3.92E−01 | 1 | Unchanged Low |
| O95755 | 129.3518 | 2.49E−01 | −4.55E−01 | 1 | Unchanged Low |
| Q9NPC3 | 127.8011 | 6.01E−01 | 2.17E−01 | 1 | Unchanged Low |
| Q9Y6E9 | 140.9006 | 5.00E−01 | 4.48E−01 | 1 | Unchanged Low |
| Q9Y5B8 | 122.4719 | 7.72E−01 | 3.30E−01 | 1 | Unchanged Low |
| P49642 | 121.2327 | 2.27E−01 | −9.47E−01 | 1 | Unchanged Low |
| O60660 | 203.1619 | −7.93E−01 | −6.34E−01 | 1 | Unchanged Low |
| Q9H251 | 151.9031 | 4.86E−01 | 7.03E−01 | 1 | Unchanged Low |
| Q9Y6C2 | 119.6097 | 5.55E−01 | −2.28E−01 | 1 | Unchanged Low |
| O95825 | 145.1487 | 1.40E−01 | −1.17E−01 | 1 | Unchanged Low |
| Q9UHG3 | 205.2993 | −6.57E−01 | −2.77E−01 | 1 | Unchanged Low |
| O00629 | 324.141 | −1.71E+00 | −8.16E−01 | 1 | Unchanged Low |
| Q15397 | 134.3527 | 5.33E−01 | 2.89E−01 | 1 | Unchanged Low |
| Q14999 | 134.6153 | 1.16E+00 | 1.28E+00 | 1 | Unchanged Low |
| Q12772 | 137.4667 | 5.34E−03 | −7.46E−01 | 1 | Unchanged Low |
| O75150 | 152.3442 | 3.01E−01 | 3.84E−01 | 1 | Unchanged Low |
| O43423 | 147.5392 | 1.84E−01 | 2.63E−02 | 1 | Unchanged Low |
| O43311 | 141.3508 | −5.12E−02 | −7.34E−01 | 1 | Unchanged Low |
| Q9NYX4 | 171.42 | −3.78E−01 | −4.91E−01 | 1 | Unchanged Low |
| Q9HD71 | 141.2517 | 3.66E−01 | 1.82E−01 | 1 | Unchanged Low |
| Q9NZH2 | 187.9443 | −4.96E−01 | −3.30E−01 | 1 | Unchanged Low |
| Q9NVH1 | 171.2625 | 2.16E−01 | 6.57E−01 | 1 | Unchanged Low |
| Q9NVG3 | 120.1783 | 7.30E−01 | 1.19E−01 | 1 | Unchanged Low |
| P52179 | 136.9676 | 2.52E−02 | −7.40E−01 | 1 | Unchanged Low |
| O94815 | 118.2939 | 7.67E−01 | 1.04E−01 | 1 | Unchanged Low |
| Q9UBY9 | 120.3216 | 6.94E−01 | 4.08E−02 | 1 | Unchanged Low |
| Q9NVX7 | 130.905 | 5.23E−01 | 1.17E−01 | 1 | Unchanged Low |
| O95704 | 124.7475 | 6.90E−01 | 2.06E−01 | 1 | Unchanged Low |
| P19801 | 131.0836 | −3.80E−02 | −1.27E+00 | 1 | Unchanged Low |
| O43709 | 140.7865 | 3.28E−01 | 6.84E−02 | 1 | Unchanged Low |
| Q12839 | 128.8096 | 5.50E−01 | 8.26E−02 | 1 | Unchanged Low |
| O43503 | 118.1934 | 7.38E−01 | 1.83E−02 | 1 | Unchanged Low |
| Q07898 | 141.7231 | 8.75E−01 | 1.01E+00 | 1 | Unchanged Low |
| O96015 | 168.8465 | 7.68E−01 | 1.41E+00 | 1 | Unchanged Low |
| Q92524 | 259.1219 | −1.30E+00 | −8.52E−01 | 1 | Unchanged Low |
| Q92692 | 260.2905 | −9.09E−01 | 6.60E−02 | 1 | Unchanged Low |
| Q9Y6Y8 | 132.2965 | 6.30E−01 | 3.34E−01 | 1 | Unchanged Low |
| Q9NZ78 | 131.7449 | 5.90E−01 | 2.41E−01 | 1 | Unchanged Low |
| Q15120 | 121.6758 | 7.42E−01 | 1.43E−01 | 1 | Unchanged Low |
| P35241 | 216.9693 | −7.74E−01 | −3.80E−01 | 1 | Unchanged Low |
| Q9Y326 | 227.0406 | −1.15E+00 | −1.17E+00 | 1 | Unchanged Low |
| Q92600 | 140.923 | 4.84E−01 | 3.30E−01 | 1 | Unchanged Low |
| O00487 | 255.4035 | −1.02E+00 | −2.60E−01 | 1 | Unchanged Low |
| Q9Y689 | 152.9706 | 2.33E−01 | 2.09E−01 | 1 | Unchanged Low |
| Q9ULB8 | 124.5785 | 8.31E−01 | 4.09E−01 | 1 | Unchanged Low |
| O60897 | 120.9939 | 8.59E−01 | 3.22E−01 | 1 | Unchanged Low |
| P51809 | 177.2043 | −4.94E−01 | −6.94E−01 | 1 | Unchanged Low |
| O75581 | 136.4067 | 4.05E−01 | 2.53E−02 | 1 | Unchanged Low |
| O43795 | 234.504 | −6.78E−01 | 1.20E−01 | 1 | Unchanged Low |
| Q14188 | 157.7494 | 5.96E−01 | 9.25E−01 | 1 | Unchanged Low |
| P06280 | 214.6169 | −1.34E−01 | 7.70E−01 | 1 | Unchanged Low |
| Q14541 | 331.895 | −1.76E+00 | −9.57E−01 | 1 | Unchanged Low |
| P57723 | 193.1283 | −1.97E−01 | 3.08E−01 | 1 | Unchanged Low |
| Q9NWU3 | 140.5056 | 7.22E−02 | −5.71E−01 | 1 | Unchanged Low |
| Q9NPC5 | 122.7442 | 6.02E−01 | −1.24E−01 | 1 | Unchanged Low |
| Q9Y3C5 | 162.3366 | 2.40E−02 | 3.68E−02 | 1 | Unchanged Low |
| Q9NWX3 | 118.9844 | 7.23E−01 | −4.44E−02 | 1 | Unchanged Low |
| P52742 | 121.6363 | 1.00E+00 | −5.68E−02 | 1 | Unchanged Low |
| Q9Y313 | 123.3545 | 8.56E−01 | 3.67E−01 | 1 | Unchanged Low |
| Q9P2W9 | 160.0555 | 3.45E−01 | 5.48E−01 | 1 | Unchanged Low |
| P14778 | 322.4754 | −6.95E−01 | 1.03E+00 | 1 | Unchanged Low |
| Q15464 | 249.6096 | −7.98E−01 | 8.24E−02 | 1 | Unchanged Low |
| O15444 | 168.4558 | −5.80E−01 | −1.33E+00 | 1 | Unchanged Low |
| P49796 | 161.0774 | −4.29E−01 | −1.15E+00 | 1 | Unchanged Low |
| Q9NUS7 | 119.7258 | 1.00E+00 | 4.77E−01 | 1 | Unchanged Low |
| P48651 | 171.5708 | −3.72E−01 | −6.06E−01 | 1 | Unchanged Low |
| Q07706 | 150.2019 | 1.94E−01 | 7.31E−01 | 1 | Unchanged Low |
| O75143 | 141.6405 | 6.20E−01 | 5.37E−01 | 1 | Unchanged Low |
| O75039 | 157.7714 | −8.78E−02 | −3.64E−01 | 1 | Unchanged Low |
| P02261 | 176.9259 | 8.10E−01 | 1.55E+00 | 1 | Unchanged Low |
| Q9H9A0 | 126.7707 | 9.27E−01 | 5.84E−01 | 1 | Unchanged Low |
| Q9NV35 | 139.1489 | 7.30E−01 | 6.33E−01 | 1 | Unchanged Low |
| Q9P1G8 | 155.3256 | 4.62E−01 | 6.08E−01 | 1 | Unchanged Low |
| P26368 | 163.126 | −3.97E−01 | −1.01E+00 | 1 | Unchanged Low |
| Q9BSZ7 | 135.6059 | 3.34E−01 | −2.35E−01 | 1 | Unchanged Low |
| Q9Y391 | 171.7067 | −4.31E−01 | −7.99E−01 | 1 | Unchanged Low |
| Q9NYR5 | 124.8579 | 7.33E−01 | 1.46E−01 | 1 | Unchanged Low |
| P05165 | 128.9755 | 7.16E−01 | 2.54E−01 | 1 | Unchanged Low |
| Q9NPF4 | 127.3658 | 6.77E−01 | 1.13E−01 | 1 | Unchanged Low |
| P16106 | 131.6683 | 7.44E−01 | 3.87E−01 | 1 | Unchanged Low |
| P35249 | 123.3812 | 6.59E−01 | −9.27E−02 | 1 | Unchanged Low |
| P28702 | 127.7227 | 1.15E+00 | 9.23E−01 | 1 | Unchanged Low |
| P49674 | 136.8564 | 7.44E−01 | 5.44E−01 | 1 | Unchanged Low |
| Q9NXW2 | 216.5041 | −5.74E−01 | −7.47E−02 | 1 | Unchanged Low |
| O14672 | 258.8185 | −1.18E+00 | −7.43E−01 | 1 | Unchanged Low |
| Q9Y5B9 | 172.4726 | 3.11E−02 | 2.04E−01 | 1 | Unchanged Low |
| Q9H9J2 | 209.7386 | −3.07E−01 | 3.10E−01 | 1 | Unchanged Low |
| P23759 | 153.0178 | 8.52E−01 | 1.11E+00 | 1 | Unchanged Low |
| Q16643 | 194.7352 | −6.66E−01 | −8.00E−01 | 1 | Unchanged Low |
| P48454 | 147.2674 | 6.57E−01 | 6.72E−01 | 1 | Unchanged Low |
| Q9Y508 | 297.9139 | −9.88E−01 | 2.52E−01 | 1 | Unchanged Low |
| P19474 | 217.1349 | −8.47E−01 | −7.20E−01 | 1 | Unchanged Low |
| P46109 | 153.202 | 1.50E−01 | −1.05E−01 | 1 | Unchanged Low |
| P54855 | 144.2152 | 2.83E−01 | −1.20E−01 | 1 | Unchanged Low |
| Q14242 | 136.8261 | 5.72E−01 | 1.98E−01 | 1 | Unchanged Low |
| Q9BSS1 | 122.482 | 9.53E−01 | 3.78E−01 | 1 | Unchanged Low |
| Q9Y3B2 | 153.2264 | 1.44E−01 | −1.23E−01 | 1 | Unchanged Low |
| P31391 | 135.9754 | 1.89E−01 | −6.72E−01 | 1 | Unchanged Low |
| O60623 | 183.0009 | −3.28E−01 | −3.12E−01 | 1 | Unchanged Low |
| Q99959 | 247.4823 | −1.11E+00 | −7.80E−01 | 1 | Unchanged Low |
| | 180.4876 | 2.72E−01 | 7.49E−01 | 1 | Unchanged Low |
| Q01658 | 165.0596 | −2.87E−01 | −7.54E−01 | 1 | Unchanged Low |
| Q9HCS4 | 133.5936 | 1.14E+00 | 1.03E+00 | 1 | Unchanged Low |
| Q9P035 | 137.5999 | 7.12E−01 | 4.65E−01 | 1 | Unchanged Low |
| Q9C0K7 | 126.1345 | 7.19E−01 | 7.24E−02 | 1 | Unchanged Low |
| Q9NYF4 | 131.7509 | 9.09E−01 | 6.11E−01 | 1 | Unchanged Low |
| P30042 | 121.6076 | 7.70E−01 | −2.03E−02 | 1 | Unchanged Low |
| O75325 | 122.705 | 5.35E−01 | −4.82E−01 | 1 | Unchanged Low |
| Q9Y670 | 118.5764 | 8.06E−01 | −8.69E−02 | 1 | Unchanged Low |
| Q9UPR8 | 126.797 | 7.34E−01 | 1.26E−01 | 1 | Unchanged Low |
| Q9Y2X7 | 165.5706 | 7.00E−02 | 5.72E−02 | 1 | Unchanged Low |
| P47872 | 184.1802 | −4.98E−02 | 2.60E−01 | 1 | Unchanged Low |
| O43353 | 276.5684 | −6.91E−01 | 5.11E−01 | 1 | Unchanged Low |
| P32321 | 149.1554 | −3.72E−01 | −1.86E+00 | 1 | Unchanged Low |
| O43612 | 143.2577 | 1.35E+00 | 1.55E+00 | 1 | Unchanged Low |
| Q92874 | 177.8201 | −3.69E−01 | −5.76E−01 | 1 | Unchanged Low |
| Q9UI90 | 215.61 | 9.22E−01 | 2.12E+00 | 1 | Unchanged Low |
| P30519 | 380.9032 | −1.79E+00 | −5.99E−01 | 1 | Unchanged Low |
| Q9NVE7 | 152.9293 | 2.57E−01 | 6.16E−02 | 1 | Unchanged Low |
| Q9P010 | 214.317 | −4.28E−01 | 1.08E−01 | 1 | Unchanged Low |
| Q9UH59 | 139.9456 | 1.99E−01 | −5.24E−01 | 1 | Unchanged Low |
| Q9UM82 | 207.1298 | −4.43E−01 | −6.20E−02 | 1 | Unchanged Low |
| Q9P2Y4 | 150.1216 | 6.84E−01 | 7.27E−01 | 1 | Unchanged Low |
| Q9GZS3 | 152.3724 | 4.47E−01 | 3.81E−01 | 1 | Unchanged Low |
| P07686 | 174.0094 | −3.52E−01 | −6.85E−01 | 1 | Unchanged Low |
| Q9NZV6 | 204.3052 | −5.05E−01 | −2.60E−01 | 1 | Unchanged Low |
| O14678 | 124.9148 | 1.13E+00 | 7.10E−01 | 1 | Unchanged Low |
| P29972 | 204.2885 | −8.61E−01 | −1.21E+00 | 1 | Unchanged Low |
| O95373 | 177.0459 | −5.38E−01 | −1.13E+00 | 1 | Unchanged Low |
| Q15436 | 120.3545 | 9.25E−01 | 1.52E−01 | 1 | Unchanged Low |
| Q9BQ67 | 141.0924 | 2.85E−01 | −3.25E−01 | 1 | Unchanged Low |
| P35321 | 156.8515 | 1.46E+00 | 1.93E+00 | 1 | Unchanged Low |
| Q9UPQ8 | 123.4833 | 1.16E+00 | 6.76E−01 | 1 | Unchanged Low |
| O43292 | 119.6038 | 8.43E−01 | −4.59E−02 | 1 | Unchanged Low |
| Q9UBM3 | 221.0285 | −7.49E−01 | −4.93E−01 | 1 | Unchanged Low |
| P19338 | 135.8947 | 2.02E−01 | −7.65E−01 | 1 | Unchanged Low |
| Q9NPE6 | 139.3341 | 6.25E−01 | 2.84E−01 | 1 | Unchanged Low |
| O00442 | 241.6967 | −6.79E−01 | 2.20E−02 | 1 | Unchanged Low |
| P28356 | 120.5873 | 1.02E+00 | 3.21E−01 | 1 | Unchanged Low |
| P49447 | 171.1671 | −2.56E−02 | −6.39E−02 | 1 | Unchanged Low |
| Q9UQA4 | 289.7702 | −4.04E−01 | 1.05E+00 | 1 | Unchanged Low |
| O15254 | 185.2853 | −1.60E−01 | −4.71E−03 | 1 | Unchanged Low |
| Q9NWW6 | 197.0098 | −2.09E−01 | 1.48E−01 | 1 | Unchanged Low |
| O75391 | 155.2376 | 1.59E−01 | −1.43E−01 | 1 | Unchanged Low |
| Q92959 | 120.1434 | 9.97E−01 | 2.43E−01 | 1 | Unchanged Low |
| O00499 | 148.8447 | 8.25E−02 | −5.50E−01 | 1 | Unchanged Low |
| Q14197 | 121.725 | 9.24E−01 | 1.62E−01 | 1 | Unchanged Low |
| Q9NXS9 | 132.3749 | 5.07E−01 | −2.36E−01 | 1 | Unchanged Low |
| O75954 | 124.8565 | 1.05E+00 | 5.18E−01 | 1 | Unchanged Low |
| Q9UJY9 | 134.9529 | 1.41E−01 | −1.05E+00 | 1 | Unchanged Low |
| P82912 | 158.0886 | 7.17E−01 | 9.03E−01 | 1 | Unchanged Low |
| Q12918 | 222.8453 | −2.51E−01 | 4.97E−01 | 1 | Unchanged Low |
| Q14789 | 154.2154 | 8.48E−01 | 1.01E+00 | 1 | Unchanged Low |
| Q04206 | 248.1566 | −9.27E−01 | −4.40E−01 | 1 | Unchanged Low |
| P19838 | 226.323 | −4.05E−01 | 2.71E−01 | 1 | Unchanged Low |
| Q92748 | 120.9562 | 7.73E−01 | −2.02E−01 | 1 | Unchanged Low |
| O95095 | 191.35 | −2.06E−01 | 5.88E−03 | 1 | Unchanged Low |
| P52209 | 177.5327 | −2.89E−01 | −5.23E−01 | 1 | Unchanged Low |
| P42566 | 175.3861 | −1.68E−01 | −3.05E−01 | 1 | Unchanged Low |
| Q13772 | 271.4641 | −6.57E−01 | 4.26E−01 | 1 | Unchanged Low |
| P43897 | 241.8156 | −9.06E−01 | −5.28E−01 | 1 | Unchanged Low |
| P09329 | 121.5057 | 7.50E−01 | −2.69E−01 | 1 | Unchanged Low |
| O75618 | 174.3849 | 2.29E−02 | 3.61E−02 | 1 | Unchanged Low |
| Q9UHI9 | 180.2862 | 4.69E−02 | 2.18E−01 | 1 | Unchanged Low |
| Q9H824 | 141.9533 | 9.76E−01 | 8.50E−01 | 1 | Unchanged Low |
| Q9UQ43 | 175.1475 | −7.35E−02 | −1.64E−01 | 1 | Unchanged Low |
| O43173 | 217.6266 | −3.29E−01 | 2.12E−01 | 1 | Unchanged Low |
| Q9BZZ2 | 170.7942 | 2.55E−01 | 3.56E−01 | 1 | Unchanged Low |
| Q92611 | 122.2106 | 8.70E−01 | −3.08E−02 | 1 | Unchanged Low |
| Q969V6 | 157.6423 | 3.76E−01 | 2.49E−01 | 1 | Unchanged Low |
| P08107 | 145.1219 | 1.98E−01 | −5.38E−01 | 1 | Unchanged Low |
| Q9BQ24 | 195.2182 | 1.96E−01 | 7.29E−01 | 1 | Unchanged Low |
| Q9H7X1 | 189.2619 | 1.85E−01 | 6.03E−01 | 1 | Unchanged Low |
| Q99616 | 135.6192 | 4.40E−01 | −3.79E−01 | 1 | Unchanged Low |
| Q92896 | 121.5361 | 8.54E−01 | −1.15E−01 | 1 | Unchanged Low |
| Q14582 | 210.1876 | −2.52E−01 | 2.12E−01 | 1 | Unchanged Low |
| Q13505 | 181.2437 | −1.73E−01 | −2.46E−01 | 1 | Unchanged Low |
| Q13722 | 135.6336 | 1.04E+00 | 7.49E−01 | 1 | Unchanged Low |
| P01308 | 218.8465 | −1.05E+00 | −1.70E+00 | 1 | Unchanged Low |
| O00405 | 238.6383 | −3.98E−01 | 3.92E−01 | 1 | Unchanged Low |
| P41215 | 214.8046 | −5.97E−01 | −4.27E−01 | 1 | Unchanged Low |
| P31314 | 151.5175 | 7.24E−01 | 6.60E−01 | 1 | Unchanged Low |
| O95427 | 125.8189 | 1.08E+00 | 4.59E−01 | 1 | Unchanged Low |
| Q12981 | 119.9152 | 1.11E+00 | 2.77E−01 | 1 | Unchanged Low |
| Q9Y5V1 | 191.3703 | −2.55E−01 | −2.03E−01 | 1 | Unchanged Low |
| Q9H4B4 | 177.8629 | 1.34E−01 | 2.59E−01 | 1 | Unchanged Low |
| Q9H6D1 | 126.9312 | 8.98E−01 | 1.72E−01 | 1 | Unchanged Low |
| O60504 | 129.0088 | 7.02E−01 | −1.33E−01 | 1 | Unchanged Low |
| Q9UNK0 | 189.0342 | −2.94E−01 | −3.53E−01 | 1 | Unchanged Low |
| O95657 | 235.2956 | −1.06E+00 | −1.23E+00 | 1 | Unchanged Low |
| P20645 | 256.0667 | −9.98E−01 | −6.03E−01 | 1 | Unchanged Low |
| O14569 | 126.62 | 5.40E−01 | −6.28E−01 | 1 | Unchanged Low |
| O15293 | 149.8523 | 1.82E−01 | −4.59E−01 | 1 | Unchanged Low |
| P00390 | 218.8246 | −6.63E−01 | −5.24E−01 | 1 | Unchanged Low |
| Q9HBV3 | 256.1517 | −3.62E−01 | 6.54E−01 | 1 | Unchanged Low |
| Q14558 | 235.5213 | −7.55E−01 | −4.09E−01 | 1 | Unchanged Low |
| O14710 | 209.7103 | −5.47E−01 | −4.66E−01 | 1 | Unchanged Low |
| Q9UJ70 | 201.9381 | 2.41E−01 | 8.66E−01 | 1 | Unchanged Low |
| Q13397 | 226.5893 | −2.71E−01 | 4.00E−01 | 1 | Unchanged Low |
| Q9NRG4 | 200.3335 | 3.23E−01 | 9.62E−01 | 1 | Unchanged Low |
| P51449 | 170.992 | −4.03E−01 | −1.28E+00 | 1 | Unchanged Low |
| Q9H768 | 128.0355 | 1.10E+00 | 5.33E−01 | 1 | Unchanged Low |
| Q14332 | 131.3078 | 1.05E+00 | 5.76E−01 | 1 | Unchanged Low |
| Q13207 | 135.2524 | 5.91E−01 | −1.44E−01 | 1 | Unchanged Low |
| Q9H6B8 | 147.5119 | 4.18E−01 | −6.19E−02 | 1 | Unchanged Low |
| O00462 | 123.2365 | 1.11E+00 | 3.59E−01 | 1 | Unchanged Low |
| Q9Y3D8 | 173.7578 | 1.05E−02 | −1.31E−01 | 1 | Unchanged Low |
| P17900 | 178.9056 | 5.28E−01 | 8.95E−01 | 1 | Unchanged Low |
| Q9P0M4 | 221.2511 | −5.35E−01 | −2.27E−01 | 1 | Unchanged Low |
| | 133.5723 | 1.03E+00 | 5.94E−01 | 1 | Unchanged Low |
| Q9NZ92 | 231.4963 | −5.71E−01 | −1.19E−01 | 1 | Unchanged Low |
| Q9NX94 | 159.7713 | 4.32E−01 | 3.07E−01 | 1 | Unchanged Low |
| P04049 | 196.2063 | −3.19E−01 | −2.98E−01 | 1 | Unchanged Low |
| Q13181 | 158.5133 | 1.04E−01 | −4.02E−01 | 1 | Unchanged Low |
| P22102 | 218.4202 | −7.09E−01 | −7.15E−01 | 1 | Unchanged Low |
| Q9NQZ8 | 138.4428 | 9.01E−01 | 5.10E−01 | 1 | Unchanged Low |
| Q9UN30 | 121.4921 | 8.93E−01 | −1.81E−01 | 1 | Unchanged Low |
| P26651 | 209.8211 | −6.60E−01 | −8.17E−01 | 1 | Unchanged Low |
| P18887 | 122.9254 | 1.08E+00 | 2.54E−01 | 1 | Unchanged Low |
| Q9Y2S0 | 170.8985 | −1.36E−02 | −3.00E−01 | 1 | Unchanged Low |
| P49023 | 276.3491 | −7.91E−01 | 9.00E−02 | 1 | Unchanged Low |
| P49321 | 126.4866 | 5.97E−01 | −6.17E−01 | 1 | Unchanged Low |
| Q13428 | 156.2838 | 1.43E−01 | −4.18E−01 | 1 | Unchanged Low |
| Q9HCS6 | 220.0848 | 3.36E−02 | 7.66E−01 | 1 | Unchanged Low |
| P20813 | 155.3947 | 5.69E−01 | 4.08E−01 | 1 | Unchanged Low |
| O00459 | 162.3275 | 3.20E−01 | 1.39E−01 | 1 | Unchanged Low |
| P50151 | 187.0796 | −3.47E−02 | 4.62E−02 | 1 | Unchanged Low |
| Q16850 | 217.3331 | −9.59E−01 | −1.61E+00 | 1 | Unchanged Low |
| Q9ULW3 | 162.0616 | 9.06E−01 | 1.09E+00 | 1 | Unchanged Low |
| Q16378 | 138.8678 | 1.15E+00 | 8.95E−01 | 1 | Unchanged Low |
| P41240 | 170.0806 | 5.89E−02 | −1.98E−01 | 1 | Unchanged Low |
| Q16649 | 235.1072 | −9.55E−01 | −1.06E+00 | 1 | Unchanged Low |
| Q9Y3M2 | 150.2264 | 1.30E+00 | 1.38E+00 | 1 | Unchanged Low |
| Q9UK58 | 229.0134 | −6.73E−02 | 7.10E−01 | 1 | Unchanged Low |
| P47874 | 201.6274 | −4.54E−01 | −5.43E−01 | 1 | Unchanged Low |
| O95620 | 205.8925 | −3.16E−01 | −1.14E−01 | 1 | Unchanged Low |
| Q9UHV2 | 163.6559 | 3.47E−01 | 1.77E−01 | 1 | Unchanged Low |
| P02810 | 328.6668 | −1.16E+00 | −6.62E−02 | 1 | Unchanged Low |
| O94903 | 190.7733 | −3.97E−01 | −7.29E−01 | 1 | Unchanged Low |
| Q9P0W6 | 169.9232 | 3.04E−01 | 2.43E−01 | 1 | Unchanged Low |
| Q9H8X4 | 294.6026 | −9.60E−01 | −6.84E−02 | 1 | Unchanged Low |
| P02786 | 254.0696 | −8.00E−01 | −3.19E−01 | 1 | Unchanged Low |
| | 249.0303 | −1.07E+00 | −1.13E+00 | 1 | Unchanged Low |
| Q99717 | 188.4757 | −3.04E−01 | −5.69E−01 | 1 | Unchanged Low |
| Q15036 | 299.6453 | −7.20E−01 | 4.34E−01 | 1 | Unchanged Low |
| P31751 | 230.4512 | −4.57E−01 | −5.57E−03 | 1 | Unchanged Low |
| P11177 | 188.1769 | −3.08E−01 | −6.08E−01 | 1 | Unchanged Low |
| Q9UEQ6 | 167.9086 | −4.31E−02 | −5.77E−01 | 1 | Unchanged Low |
| Q16531 | 206.5357 | −1.48E−01 | 1.60E−01 | 1 | Unchanged Low |
| P28698 | 122.3029 | 1.28E+00 | 4.70E−01 | 1 | Unchanged Low |
| P41968 | 232.6783 | −2.81E−01 | 3.49E−01 | 1 | Unchanged Low |
| P54920 | 252.9933 | −7.24E−01 | −2.03E−01 | 1 | Unchanged Low |
| Q99487 | 218.5222 | −2.99E−01 | 8.00E−02 | 1 | Unchanged Low |
| Q9H0C8 | 138.517 | 6.20E−01 | −1.49E−01 | 1 | Unchanged Low |
| P17252 | 122.7553 | 7.78E−01 | −5.50E−01 | 1 | Unchanged Low |
| P06493 | 123.4207 | 7.81E−01 | −5.05E−01 | 1 | Unchanged Low |
| O00139 | 177.1988 | 4.05E−01 | 5.48E−01 | 1 | Unchanged Low |
| P54259 | 167.773 | 1.69E−01 | −1.26E−01 | 1 | Unchanged Low |
| Q99757 | 219.4587 | −7.32E−01 | −9.21E−01 | 1 | Unchanged Low |
| Q9NW68 | 178.6551 | 7.93E−01 | 1.17E+00 | 1 | Unchanged Low |
| P16106 | 152.9203 | 4.33E−01 | −3.69E−02 | 1 | Unchanged Low |
| P40692 | 131.0441 | 9.15E−01 | 1.30E−01 | 1 | Unchanged Low |
| Q99798 | 364.1757 | −1.72E+00 | −1.08E+00 | 1 | Unchanged Low |
| P53618 | 175.716 | −3.54E−01 | −1.23E+00 | 1 | Unchanged Low |
| Q9UJ69 | 219.0188 | −1.80E−01 | 2.93E−01 | 1 | Unchanged Low |
| Q9NRM2 | 215.6334 | −3.18E−01 | −3.70E−02 | 1 | Unchanged Low |
| Q92879 | 200.6744 | −2.16E−01 | −1.38E−01 | 1 | Unchanged Low |
| O75399 | 214.4669 | −1.95E−02 | 4.88E−01 | 1 | Unchanged Low |
| Q9BZE4 | 184.3092 | 3.08E−01 | 5.03E−01 | 1 | Unchanged Low |
| Q9NXB2 | 243.8822 | −4.67E−01 | 1.38E−01 | 1 | Unchanged Low |
| O95424 | 158.2861 | 8.62E−01 | 8.35E−01 | 1 | Unchanged Low |
| P54577 | 146.9084 | 3.66E−01 | −4.36E−01 | 1 | Unchanged Low |
| O43692 | 150.9471 | 8.73E−01 | 6.65E−01 | 1 | Unchanged Low |
| O60507 | 171.4862 | −1.67E−02 | −4.83E−01 | 1 | Unchanged Low |
| Q9BYS9 | 134.7877 | 9.22E−01 | 2.36E−01 | 1 | Unchanged Low |
| P32780 | 209.0492 | −4.59E−01 | −5.25E−01 | 1 | Unchanged Low |
| Q14004 | 146.1954 | 1.02E+00 | 7.68E−01 | 1 | Unchanged Low |
| Q15910 | 235.9636 | −1.75E−01 | 5.21E−01 | 1 | Unchanged Low |
| P47974 | 179.8162 | 8.96E−03 | −2.05E−01 | 1 | Unchanged Low |
| Q13177 | 262.9597 | −9.61E−01 | −6.63E−01 | 1 | Unchanged Low |
| | 188.6409 | −4.11E−01 | −1.00E+00 | 1 | Unchanged Low |
| P98172 | 140.4039 | 6.25E−01 | −1.75E−01 | 1 | Unchanged Low |
| Q9BQB1 | 158.4309 | 7.36E−01 | 5.91E−01 | 1 | Unchanged Low |
| O75472 | 206.8756 | −2.58E−01 | −1.58E−01 | 1 | Unchanged Low |
| O75608 | 307.2824 | −9.11E−01 | 6.48E−02 | 1 | Unchanged Low |
| O00193 | 232.7223 | −2.36E−01 | 3.42E−01 | 1 | Unchanged Low |
| Q14452 | 164.6255 | 2.80E−01 | −9.75E−02 | 1 | Unchanged Low |
| P55268 | 155.8763 | 6.08E−01 | 2.81E−01 | 1 | Unchanged Low |
| Q9P006 | 152.156 | 7.33E−01 | 3.98E−01 | 1 | Unchanged Low |
| Q9Y365 | 175.2567 | 2.65E−01 | 1.52E−01 | 1 | Unchanged Low |
| Q9NQ34 | 165.3815 | 4.02E−01 | 1.59E−01 | 1 | Unchanged Low |
| Q9NWY4 | 123.5579 | 1.45E+00 | 6.46E−01 | 1 | Unchanged Low |
| Q9UBS8 | 221.9109 | −2.54E−01 | 1.13E−01 | 1 | Unchanged Low |
| Q9UBS4 | 182.7428 | 7.25E−02 | −5.40E−02 | 1 | Unchanged Low |
| Q12797 | 126.3618 | 9.19E−01 | −2.27E−01 | 1 | Unchanged Low |
| Q9H4I9 | 192.7801 | 1.93E−01 | 3.89E−01 | 1 | Unchanged Low |
| O14907 | 315.6313 | −8.75E−01 | 1.97E−01 | 1 | Unchanged Low |
| Q14673 | 135.533 | 9.82E−01 | 2.74E−01 | 1 | Unchanged Low |
| Q9NWB7 | 281.455 | −1.56E+00 | −2.75E+00 | 1 | Unchanged Low |
| Q9BVH4 | 175.4765 | 2.23E−01 | 3.72E−02 | 1 | Unchanged Low |
| P22897 | 123.4215 | 1.32E+00 | 3.78E−01 | 1 | Unchanged Low |
| P19012 | 156.5003 | 3.94E−01 | −1.69E−01 | 1 | Unchanged Low |
| Q14738 | 200.124 | −2.19E−01 | −2.94E−01 | 1 | Unchanged Low |
| O95152 | 195.3686 | −3.47E−02 | −1.53E−02 | 1 | Unchanged Low |
| P78426 | 248.2544 | −4.06E−02 | 8.31E−01 | 1 | Unchanged Low |
| Q14790 | 200.6718 | 6.29E−01 | 1.19E+00 | 1 | Unchanged Low |
| P07711 | 150.5861 | 4.94E−01 | −1.75E−01 | 1 | Unchanged Low |
| P32942 | 157.6897 | 4.61E−01 | −8.57E−03 | 1 | Unchanged Low |
| | 171.3764 | 4.00E−02 | −5.29E−01 | 1 | Unchanged Low |
| Q9NWS0 | 127.6547 | 1.26E+00 | 3.99E−01 | 1 | Unchanged Low |
| Q9NWZ5 | 242.9739 | 1.42E−01 | 1.02E+00 | 1 | Unchanged Low |
| O75792 | 131.5441 | 9.56E−01 | −1.12E−02 | 1 | Unchanged Low |
| P05141 | 320.3918 | −1.22E+00 | −5.47E−01 | 1 | Unchanged Low |
| Q9NX38 | 165.7012 | 4.72E−01 | 1.95E−01 | 1 | Unchanged Low |
| Q9Y682 | 220.1941 | 1.21E−02 | 4.86E−01 | 1 | Unchanged Low |
| P78381 | 226.4009 | −4.97E−01 | −4.14E−01 | 1 | Unchanged Low |
| Q9UMP5 | 214.9641 | −4.67E−01 | −5.97E−01 | 1 | Unchanged Low |
| Q9P0Q0 | 188.838 | 5.95E−01 | 8.96E−01 | 1 | Unchanged Low |
| Q9UBQ3 | 185.7473 | 4.72E−02 | −1.39E−01 | 1 | Unchanged Low |
| P27540 | 231.071 | −2.91E−01 | 1.07E−01 | 1 | Unchanged Low |
| Q9Y2W1 | 201.3236 | 2.89E−01 | 6.30E−01 | 1 | Unchanged Low |
| P82267 | 208.571 | −2.07E−02 | 2.20E−01 | 1 | Unchanged Low |
| P00918 | 216.9569 | −5.33E−01 | −7.25E−01 | 1 | Unchanged Low |
| Q9Y663 | 184.9847 | −4.02E−01 | −1.38E+00 | 1 | Unchanged Low |
| Q9NX40 | 187.7106 | 7.14E−01 | 1.05E+00 | 1 | Unchanged Low |
| P50222 | 195.0839 | 2.50E−01 | 4.34E−01 | 1 | Unchanged Low |
| Q13610 | 229.4375 | 2.90E−01 | 1.04E+00 | 1 | Unchanged Low |
| P49591 | 139.6467 | 3.68E−01 | −1.13E+00 | 1 | Unchanged Low |
| P04554 | 269.3189 | −6.77E−01 | −8.89E−02 | 1 | Unchanged Low |
| P02538 | 233.9139 | −2.09E−01 | 2.80E−01 | 1 | Unchanged Low |
| Q13637 | 286.1382 | −9.91E−01 | −5.43E−01 | 1 | Unchanged Low |
| P39086 | 197.2285 | 9.83E−02 | 1.88E−01 | 1 | Unchanged Low |
| Q9H8F9 | 228.8927 | −5.40E−01 | −5.06E−01 | 1 | Unchanged Low |
| Q99460 | 241.22 | −5.96E−01 | −3.89E−01 | 1 | Unchanged Low |
| Q9UKP6 | 184.3366 | −1.11E−01 | −5.86E−01 | 1 | Unchanged Low |
| Q16589 | 170.3526 | 9.88E−01 | 1.10E+00 | 1 | Unchanged Low |
| O95316 | 162.324 | 4.48E−01 | −2.40E−03 | 1 | Unchanged Low |
| O14545 | 241.5146 | −2.69E−01 | 2.74E−01 | 1 | Unchanged Low |
| Q9Y249 | 204.4863 | 5.18E−02 | 2.31E−01 | 1 | Unchanged Low |
| Q92794 | 170.3523 | 5.64E−01 | 4.13E−01 | 1 | Unchanged Low |
| P36894 | 194.5315 | 4.11E−01 | 6.59E−01 | 1 | Unchanged Low |
| P79525 | 148.0291 | 7.17E−01 | 4.53E−02 | 1 | Unchanged Low |
| O14947 | 281.2972 | −8.57E−01 | −3.47E−01 | 1 | Unchanged Low |
| Q12899 | 212.5994 | 7.89E−02 | 4.05E−01 | 1 | Unchanged Low |
| | 142.4645 | 1.13E+00 | 5.79E−01 | 1 | Unchanged Low |
| Q9UIJ5 | 362.7132 | −1.13E+00 | 5.01E−02 | 1 | Unchanged Low |
| P46821 | 152.4972 | 7.21E−01 | 1.70E−01 | 1 | Unchanged Low |
| O60906 | 254.5351 | −5.00E−01 | −7.08E−03 | 1 | Unchanged Low |
| P04040 | 199.8034 | 3.15E−01 | 5.71E−01 | 1 | Unchanged Low |
| O60709 | 168.4792 | 1.23E+00 | 1.37E+00 | 1 | Unchanged Low |
| Q14714 | 242.1486 | −9.20E−01 | −1.36E+00 | 1 | Unchanged Low |
| P39687 | 178.3873 | −1.80E−01 | −1.07E+00 | 1 | Unchanged Low |
| O75341 | 187.1088 | 3.99E−01 | 4.62E−01 | 1 | Unchanged Low |
| Q9NUN1 | 182.989 | 3.94E−01 | 3.60E−01 | 1 | Unchanged Low |
| Q9UHR5 | 256.9591 | −1.65E−01 | 6.16E−01 | 1 | Unchanged Low |
| Q9UMX3 | 187.4707 | 1.18E−01 | −7.26E−02 | 1 | Unchanged Low |
| Q02297 | 175.0464 | 1.17E+00 | 1.39E+00 | 1 | Unchanged Low |
| Q9BQ83 | 300.7124 | −5.89E−01 | 3.95E−01 | 1 | Unchanged Low |
| O43491 | 203.6633 | −3.98E−01 | −8.84E−01 | 1 | Unchanged Low |
| Q92922 | 216.0309 | −3.18E−01 | −3.64E−01 | 1 | Unchanged Low |
| Q16099 | 229.9201 | 1.17E−02 | 5.26E−01 | 1 | Unchanged Low |
| O95328 | 231.7737 | −3.29E−01 | −7.56E−02 | 1 | Unchanged Low |
| Q06265 | 183.9827 | 2.73E−01 | 1.25E−01 | 1 | Unchanged Low |
| Q01780 | 223.1866 | 3.82E−02 | 4.57E−01 | 1 | Unchanged Low |
| Q9NPF9 | 207.9337 | −3.14E−01 | −5.70E−01 | 1 | Unchanged Low |
| Q9P1U0 | 154.8483 | 8.72E−01 | 4.48E−01 | 1 | Unchanged Low |
| Q13480 | 174.7367 | 2.43E−01 | −1.98E−01 | 1 | Unchanged Low |
| O95205 | 202.2261 | 5.72E−01 | 9.62E−01 | 1 | Unchanged Low |
| O76091 | 192.2638 | 6.67E−02 | −1.17E−01 | 1 | Unchanged Low |
| Q9Y364 | 244.4561 | −3.22E−02 | 6.28E−01 | 1 | Unchanged Low |
| P51116 | 210.8166 | −8.49E−02 | −2.12E−02 | 1 | Unchanged Low |
| P49915 | 164.6237 | 2.15E−01 | −6.13E−01 | 1 | Unchanged Low |
| Q9UHR3 | 173.3257 | 7.64E−01 | 7.09E−01 | 1 | Unchanged Low |
| Q15007 | 233.0967 | −2.15E−01 | 1.22E−01 | 1 | Unchanged Low |
| P24941 | 172.2468 | 1.05E+00 | 1.12E+00 | 1 | Unchanged Low |
| P53634 | 126.6548 | 1.07E+00 | −3.22E−01 | 1 | Unchanged Low |
| Q13123 | 155.5636 | 9.16E−01 | 4.95E−01 | 1 | Unchanged Low |
| Q9NUT6 | 351.7208 | −4.77E−01 | 9.85E−01 | 1 | Unchanged Low |
| Q9Y279 | 262.6019 | −7.99E−01 | −6.59E−01 | 1 | Unchanged Low |
| O75319 | 220.2678 | −2.87E−01 | −2.84E−01 | 1 | Unchanged Low |
| Q9NYH9 | 258.6032 | −4.85E−01 | −1.99E−02 | 1 | Unchanged Low |
| Q9UNF0 | 331.0599 | −1.09E+00 | −3.29E−01 | 1 | Unchanged Low |
| O60437 | 316.8358 | −1.11E+00 | −5.61E−01 | 1 | Unchanged Low |
| Q9NZN8 | 206.5153 | 1.17E−01 | 2.41E−01 | 1 | Unchanged Low |
| Q14232 | 229.9659 | −4.12E−01 | −3.79E−01 | 1 | Unchanged Low |
| O00142 | 159.1674 | 7.24E−01 | 2.35E−01 | 1 | Unchanged Low |
| O60443 | 197.4327 | 7.53E−01 | 1.11E+00 | 1 | Unchanged Low |
| O95406 | 175.4375 | 1.84E−01 | −3.95E−01 | 1 | Unchanged Low |
| Q12841 | 128.3877 | 1.35E+00 | 2.72E−01 | 1 | Unchanged Low |
| O15217 | 125.424 | 1.40E+00 | 2.20E−01 | 1 | Unchanged Low |
| P10645 | 201.8922 | 8.38E−01 | 1.29E+00 | 1 | Unchanged Low |
| Q9HA66 | 199.1923 | 6.24E−02 | −4.27E−02 | 1 | Unchanged Low |
| Q9Y439 | 230.9065 | 2.37E−01 | 8.19E−01 | 1 | Unchanged Low |
| Q9Y386 | 259.7061 | −6.48E−01 | −4.06E−01 | 1 | Unchanged Low |
| P53990 | 279.676 | −1.46E−01 | 8.01E−01 | 1 | Unchanged Low |
| O95865 | 163.3476 | 7.35E−01 | 3.29E−01 | 1 | Unchanged Low |
| | 193.5142 | 5.44E−01 | 6.86E−01 | 1 | Unchanged Low |
| Q15834 | 251.6031 | −6.24E−01 | −5.06E−01 | 1 | Unchanged Low |
| P18846 | 295.2222 | −1.18E+00 | −1.19E+00 | 1 | Unchanged Low |
| Q14349 | 174.3142 | 5.87E−01 | 3.37E−01 | 1 | Unchanged Low |
| Q9NVM5 | 140.794 | 1.09E+00 | 2.30E−01 | 1 | Unchanged Low |
| P12955 | 178.2362 | 6.43E−01 | 5.20E−01 | 1 | Unchanged Low |
| Q99700 | 213.1956 | 1.30E−01 | 3.27E−01 | 1 | Unchanged Low |
| Q9UJA2 | 219.8275 | 6.40E−01 | 1.23E+00 | 1 | Unchanged Low |
| Q02246 | 128.7469 | 9.55E−01 | −6.50E−01 | 1 | Unchanged Low |
| P48553 | 198.4177 | 5.00E−01 | 6.84E−01 | 1 | Unchanged Low |
| Q99639 | 243.5051 | −1.02E−01 | 3.92E−01 | 1 | Unchanged Low |
| Q9NQX5 | 201.225 | 1.58E−01 | 1.30E−01 | 1 | Unchanged Low |
| Q99799 | 196.7655 | 3.13E−02 | −2.32E−01 | 1 | Unchanged Low |
| Q9BZV1 | 191.2074 | 7.17E−01 | 8.86E−01 | 1 | Unchanged Low |
| Q03113 | 154.1344 | 5.82E−01 | −3.22E−01 | 1 | Unchanged Low |
| P01130 | 301.7776 | −5.17E−01 | 3.87E−01 | 1 | Unchanged Low |
| Q9UHN6 | 174.2344 | 2.08E−01 | −4.89E−01 | 1 | Unchanged Low |
| Q99738 | 205.0214 | 7.00E−02 | 2.10E−02 | 1 | Unchanged Low |
| Q9Y576 | 136.0527 | 8.00E−01 | −6.69E−01 | 1 | Unchanged Low |
| P02304 | 161.1406 | 1.38E+00 | 1.24E+00 | 1 | Unchanged Low |
| P01118 | 219.9522 | 1.10E−02 | 1.93E−01 | 1 | Unchanged Low |
| P15924 | 241.3819 | −2.44E−01 | 7.48E−02 | 1 | Unchanged Low |
| O43597 | 161.8627 | 8.21E−01 | 3.75E−01 | 1 | Unchanged Low |
| P09622 | 263.2128 | −7.14E−01 | −5.77E−01 | 1 | Unchanged Low |
| Q08257 | 342.4839 | −1.28E+00 | −7.73E−01 | 1 | Unchanged Low |
| Q9UHY8 | 131.2705 | 1.29E+00 | 1.31E−01 | 1 | Unchanged Low |
| P10646 | 422.403 | −1.49E+00 | −3.81E−01 | 1 | Unchanged Low |
| Q92685 | 208.1394 | 1.21E−01 | 1.53E−01 | 1 | Unchanged Low |
| P34949 | 149.8251 | 1.05E+00 | 4.00E−01 | 1 | Unchanged Low |
| Q9NPE9 | 153.7649 | 1.21E+00 | 7.81E−01 | 1 | Unchanged Low |
| Q99853 | 153.0918 | 5.87E−01 | −4.25E−01 | 1 | Unchanged Low |
| Q9HD41 | 241.047 | −3.97E−01 | −2.85E−01 | 1 | Unchanged Low |
| Q16625 | 470.2634 | −2.01E+00 | −1.35E+00 | 1 | Unchanged Low |
| P45973 | 167.3186 | 3.70E−01 | −4.13E−01 | 1 | Unchanged Low |
| Q9NVI9 | 161.8885 | 6.47E−01 | 3.42E−03 | 1 | Unchanged Low |
| P37173 | 178.0564 | 9.24E−01 | 8.90E−01 | 1 | Unchanged Low |
| Q9UKF7 | 277.9863 | −6.44E−01 | −2.18E−01 | 1 | Unchanged Low |
| P16870 | 144.0377 | 1.38E+00 | 7.40E−01 | 1 | Unchanged Low |
| P00374 | 140.6638 | 5.22E−01 | −1.32E+00 | 1 | Unchanged Low |
| P19957 | 333.0661 | 7.60E−02 | 1.52E+00 | 1 | Unchanged Low |
| O75962 | 215.0154 | −1.28E−01 | −2.45E−01 | 1 | Unchanged Low |
| P16050 | 196.6194 | −4.31E−01 | −1.72E+00 | 1 | Unchanged Low |
| Q9Y3D2 | 213.0068 | 9.09E−02 | 1.57E−01 | 1 | Unchanged Low |
| O00505 | 295.4165 | −7.45E−01 | −1.96E−01 | 1 | Unchanged Low |
| Q9NZD8 | 220.0079 | −5.62E−02 | 6.21E−04 | 1 | Unchanged Low |
| P46937 | 233.924 | −3.10E−02 | 2.93E−01 | 1 | Unchanged Low |
| P53384 | 236.5867 | −3.45E−01 | −2.91E−01 | 1 | Unchanged Low |
| Q9UI28 | 160.6424 | 8.34E−01 | 2.84E−01 | 1 | Unchanged Low |
| Q14651 | 345.2493 | −1.27E+00 | −7.72E−01 | 1 | Unchanged Low |
| P10914 | 306.8604 | −5.32E−01 | 3.46E−01 | 1 | Unchanged Low |
| P21281 | 383.914 | −4.76E−01 | 1.09E+00 | 1 | Unchanged Low |
| P53701 | 293.8906 | −2.28E−01 | 7.19E−01 | 1 | Unchanged Low |
| Q9Y2B1 | 147.2535 | 1.06E+00 | 2.50E−01 | 1 | Unchanged Low |
| Q14166 | 274.7657 | −6.26E−01 | −2.68E−01 | 1 | Unchanged Low |
| Q15738 | 213.0647 | −2.31E−01 | −5.69E−01 | 1 | Unchanged Low |
| Q16586 | 234.3861 | −7.31E−01 | −1.47E+00 | 1 | Unchanged Low |
| Q9Y6J8 | 272.4953 | −3.48E−01 | 2.48E−01 | 1 | Unchanged Low |
| Q9H5F2 | 250.8425 | 8.24E−02 | 7.03E−01 | 1 | Unchanged Low |
| Q05655 | 310.8344 | −9.58E−01 | −5.00E−01 | 1 | Unchanged Low |
| Q9NXU7 | 230.6008 | −1.54E−01 | −3.21E−02 | 1 | Unchanged Low |
| O15498 | 215.7355 | 1.98E−01 | 3.54E−01 | 1 | Unchanged Low |
| Q92664 | 242.5829 | −5.54E−01 | −7.23E−01 | 1 | Unchanged Low |
| Q9Y223 | 239.9617 | 6.67E−02 | 5.11E−01 | 1 | Unchanged Low |
| O95816 | 132.1461 | 1.36E+00 | 1.53E−01 | 1 | Unchanged Low |
| Q9H0K5 | 244.8706 | 1.72E−01 | 7.45E−01 | 1 | Unchanged Low |
| Q9BTJ1 | 253.7125 | 7.24E−01 | 1.64E+00 | 1 | Unchanged Low |
| Q9NRR5 | 185.8791 | 6.60E−01 | 5.40E−01 | 1 | Unchanged Low |
| Q9Y5L3 | 256.4569 | −1.07E−01 | 4.24E−01 | 1 | Unchanged Low |
| O75351 | 267.9692 | −3.29E−01 | 1.78E−01 | 1 | Unchanged Low |
| O75443 | 184.2202 | 2.54E−01 | −2.86E−01 | 1 | Unchanged Low |
| Q9H7X4 | 286.2489 | −2.94E−01 | 4.71E−01 | 1 | Unchanged Low |
| O00255 | 156.9374 | 7.01E−01 | −2.02E−01 | 1 | Unchanged Low |
| Q9BV94 | 284.912 | −5.42E−01 | −1.37E−02 | 1 | Unchanged Low |
| O14813 | 255.131 | −4.57E−02 | 4.99E−01 | 1 | Unchanged Low |
| P35269 | 225.4377 | −1.49E−01 | −1.98E−01 | 1 | Unchanged Low |
| Q12891 | 287.6363 | −5.88E−01 | −9.12E−02 | 1 | Unchanged Low |
| O15162 | 419.5068 | −9.98E−01 | 4.39E−01 | 1 | Unchanged Low |
| Q14469 | 207.739 | 5.48E−01 | 7.31E−01 | 1 | Unchanged Low |
| P06241 | 136.7385 | 1.06E+00 | −3.67E−01 | 1 | Unchanged Low |
| Q9BVG9 | 215.8454 | −2.60E−02 | −1.71E−01 | 1 | Unchanged Low |
| Q9P0P9 | 315.5648 | −3.84E−01 | 5.78E−01 | 1 | Unchanged Low |
| Q9UMF9 | 224.2505 | 1.01E−01 | 2.24E−01 | 1 | Unchanged Low |
| Q03527 | 316.1666 | −9.88E−01 | −6.36E−01 | 1 | Unchanged Low |
| P31153 | 200.5541 | −1.38E−01 | −8.75E−01 | 1 | Unchanged Low |
| Q04741 | 217.4148 | −1.10E−01 | −3.46E−01 | 1 | Unchanged Low |
| Q13153 | 247.6258 | −1.26E−01 | 1.87E−01 | 1 | Unchanged Low |
| Q9Y388 | 168.2598 | 1.15E+00 | 8.44E−01 | 1 | Unchanged Low |
| Q9HC14 | 193.9429 | 4.92E−01 | 3.31E−01 | 1 | Unchanged Low |
| P30281 | 166.0499 | 1.05E+00 | 6.39E−01 | 1 | Unchanged Low |
| Q9UFK2 | 188.8934 | 7.48E−01 | 6.60E−01 | 1 | Unchanged Low |
| Q13011 | 278.2904 | −5.95E−01 | −3.07E−01 | 1 | Unchanged Low |
| P09601 | 278.3661 | 2.63E−01 | 1.19E+00 | 1 | Unchanged Low |
| Q9BVZ8 | 159.346 | 8.23E−01 | 6.71E−03 | 1 | Unchanged Low |
| Q13889 | 179.5227 | 9.99E−01 | 8.50E−01 | 1 | Unchanged Low |
| P49703 | 147.0234 | 8.35E−01 | −4.62E−01 | 1 | Unchanged Low |
| Q9GZU7 | 226.5474 | −4.46E−01 | −1.03E+00 | 1 | Unchanged Low |
| P10253 | 189.0733 | 2.59E−01 | −2.69E−01 | 1 | Unchanged Low |
| Q9Y3V5 | 318.1897 | −4.37E−01 | 4.89E−01 | 1 | Unchanged Low |
| O14773 | 202.7117 | 4.04E−01 | 3.35E−01 | 1 | Unchanged Low |
| P22061 | 168.6387 | 9.26E−01 | 4.66E−01 | 1 | Unchanged Low |
| P14868 | 140.1468 | 9.42E−01 | −5.85E−01 | 1 | Unchanged Low |
| Q14938 | 134.9545 | 1.25E+00 | −1.55E−01 | 1 | Unchanged Low |
| O75968 | 219.072 | 6.29E−02 | −1.06E−03 | 1 | Unchanged Low |
| Q9H779 | 225.7323 | 7.06E−01 | 1.18E+00 | 1 | Unchanged Low |
| Q9BX72 | 188.9468 | 4.18E−01 | 2.61E−02 | 1 | Unchanged Low |
| O43563 | 169.4168 | 7.56E−01 | 1.53E−01 | 1 | Unchanged Low |
| O15142 | 230.3093 | −1.47E−01 | −2.22E−01 | 1 | Unchanged Low |
| Q9UJ41 | 171.2118 | 8.94E−01 | 4.38E−01 | 1 | Unchanged Low |
| P05231 | 379.972 | −8.48E−02 | 1.49E+00 | 1 | Unchanged Low |
| Q9Y2H0 | 271.9604 | −3.04E−01 | 1.48E−01 | 1 | Unchanged Low |
| Q9UJT9 | 269.5315 | −3.44E−01 | 2.98E−02 | 1 | Unchanged Low |
| Q9Y306 | 187.7391 | 6.28E−01 | 3.69E−01 | 1 | Unchanged Low |
| Q9Y6A6 | 190.8287 | 3.79E−01 | −2.97E−02 | 1 | Unchanged Low |
| O75300 | 133.1515 | 1.20E+00 | −4.21E−01 | 1 | Unchanged Low |
| P51798 | 263.0106 | −2.86E−01 | 1.98E−02 | 1 | Unchanged Low |
| Q14839 | 164.8091 | 1.12E+00 | 6.20E−01 | 1 | Unchanged Low |
| O60299 | 232.6614 | 7.85E−01 | 1.35E+00 | 1 | Unchanged Low |
| Q9Y6F1 | 292.1577 | −1.24E−01 | 6.80E−01 | 1 | Unchanged Low |
| O15212 | 256.7661 | −1.30E−01 | 2.22E−01 | 1 | Unchanged Low |
| Q9Y2W2 | 289.1386 | −5.51E−01 | −1.42E−01 | 1 | Unchanged Low |
| Q9UKN5 | 187.2825 | −1.44E−01 | −1.65E+00 | 1 | Unchanged Low |
| Q00978 | 312.2774 | −4.09E−01 | 4.13E−01 | 1 | Unchanged Low |
| Q9UHY0 | 263.8122 | −3.71E−01 | −1.50E−01 | 1 | Unchanged Low |
| Q9Y248 | 130.5748 | 1.31E+00 | −3.95E−01 | 1 | Unchanged Low |
| O96019 | 267.9206 | 3.79E−03 | 5.92E−01 | 1 | Unchanged Low |
| P10276 | 200.7048 | −1.22E−01 | −1.03E+00 | 1 | Unchanged Low |
| Q9BUC7 | 254.8265 | 7.52E−01 | 1.55E+00 | 1 | Unchanged Low |
| Q9HAC1 | 156.9785 | 6.81E−01 | −5.57E−01 | 1 | Unchanged Low |
| Q93078 | 204.3058 | 3.56E−01 | 1.72E−01 | 1 | Unchanged Low |
| P08572 | 129.2665 | 1.72E+00 | 3.15E−01 | 1 | Unchanged Low |
| P15056 | 199.7155 | 2.51E−01 | −1.59E−01 | 1 | Unchanged Low |
| Q15543 | 238.5733 | 5.70E−02 | 2.38E−01 | 1 | Unchanged Low |
| O14901 | 262.1749 | −2.63E−01 | −4.43E−03 | 1 | Unchanged Low |
| | 267.6704 | −4.37E−01 | −2.87E−01 | 1 | Unchanged Low |
| P51965 | 386.4499 | −1.32E−01 | 1.40E+00 | 1 | Unchanged Low |
| Q9Y3R2 | 263.1006 | −5.43E−01 | −6.31E−01 | 1 | Unchanged Low |
| O94829 | 213.3188 | 5.30E−01 | 6.17E−01 | 1 | Unchanged Low |
| Q92859 | 211.5326 | 6.04E−01 | 7.01E−01 | 1 | Unchanged Low |
| Q12846 | 216.5887 | −4.74E−01 | −1.80E+00 | 1 | Unchanged Low |
| P01286 | 244.6261 | −2.83E−02 | 1.47E−01 | 1 | Unchanged Low |
| Q9UH92 | 200.1328 | 4.33E−01 | 1.72E−01 | 1 | Unchanged Low |
| O75323 | 193.2783 | 3.89E−01 | −8.78E−02 | 1 | Unchanged Low |
| Q9UQ33 | 156.5563 | 1.20E+00 | 3.93E−01 | 1 | Unchanged Low |
| Q9Y3A7 | 282.2981 | −7.79E−01 | −9.34E−01 | 1 | Unchanged Low |
| Q9Y2Z0 | 224.9662 | 3.44E−01 | 4.71E−01 | 1 | Unchanged Low |
| P56211 | 299.2676 | −2.41E−01 | 4.81E−01 | 1 | Unchanged Low |
| P21127 | 232.4012 | 5.59E−01 | 9.32E−01 | 1 | Unchanged Low |
| Q07866 | 299.1796 | −1.19E−01 | 6.82E−01 | 1 | Unchanged Low |
| Q9BTI3 | 231.0835 | 3.62E−02 | 8.33E−03 | 1 | Unchanged Low |
| Q9H2J4 | 243.2412 | −2.27E−02 | 1.00E−01 | 1 | Unchanged Low |
| Q9NXE5 | 275.9024 | 2.79E−01 | 1.04E+00 | 1 | Unchanged Low |
| Q92572 | 176.703 | 1.45E+00 | 1.27E+00 | 1 | Unchanged Low |
| Q9NXT6 | 255.685 | −1.14E−01 | 1.20E−01 | 1 | Unchanged Low |
| O15321 | 252.5647 | −3.24E−01 | −3.79E−01 | 1 | Unchanged Low |
| O15383 | 360.7235 | −7.73E−01 | 1.39E−01 | 1 | Unchanged Low |
| O60403 | 245.2774 | −1.12E−02 | 1.41E−01 | 1 | Unchanged Low |
| P49354 | 201.1077 | 4.40E−01 | 1.54E−01 | 1 | Unchanged Low |
| P48039 | 249.7986 | −3.69E−01 | −5.51E−01 | 1 | Unchanged Low |
| O43562 | 158.9844 | 1.03E+00 | 1.20E−01 | 1 | Unchanged Low |
| Q9HC89 | 230.2337 | 1.56E−01 | 1.93E−01 | 1 | Unchanged Low |
| P00790 | 253.0491 | −1.13E−01 | 5.77E−02 | 1 | Unchanged Low |
| Q9NVP1 | 134.724 | 1.59E+00 | 1.83E−01 | 1 | Unchanged Low |
| Q9UBN1 | 289.0967 | −1.88E−01 | 4.16E−01 | 1 | Unchanged Low |
| P35659 | 131.6712 | 1.57E+00 | −4.06E−02 | 1 | Unchanged Low |
| P54252 | 222.5126 | 5.98E−01 | 8.03E−01 | 1 | Unchanged Low |
| Q14152 | 289.959 | −6.19E−01 | −4.43E−01 | 1 | Unchanged Low |
| Q9Y314 | 193.3594 | 9.44E−01 | 8.31E−01 | 1 | Unchanged Low |
| Q9NRC7 | 233.7238 | 8.04E−01 | 1.27E+00 | 1 | Unchanged Low |
| P02533 | 210.4458 | 2.25E−01 | −9.66E−02 | 1 | Unchanged Low |
| O60907 | 194.8701 | 4.34E−01 | −4.19E−02 | 1 | Unchanged Low |
| P43155 | 238.6829 | −2.78E−01 | −6.03E−01 | 1 | Unchanged Low |
| Q15077 | 292.1056 | −7.12E−01 | −6.52E−01 | 1 | Unchanged Low |
| Q12840 | 252.8959 | −7.25E−04 | 2.43E−01 | 1 | Unchanged Low |
| P26599 | 247.8557 | 5.71E−01 | 1.10E+00 | 1 | Unchanged Low |
| Q9Y316 | 338.8101 | −9.17E−01 | −4.62E−01 | 1 | Unchanged Low |
| P24394 | 292.7071 | −3.75E−01 | 9.24E−02 | 1 | Unchanged Low |
| P32418 | 181.3894 | 4.77E−01 | −3.56E−01 | 1 | Unchanged Low |
| P42694 | 183.6122 | 5.27E−01 | −1.79E−01 | 1 | Unchanged Low |
| O60755 | 223.2368 | 5.54E−01 | 7.22E−01 | 1 | Unchanged Low |
| Q16204 | 218.6477 | 3.14E−01 | 2.29E−01 | 1 | Unchanged Low |
| Q12980 | 198.8702 | 3.79E−01 | −7.95E−02 | 1 | Unchanged Low |
| P08559 | 273.612 | −6.48E−01 | −8.63E−01 | 1 | Unchanged Low |
| Q9Y2U9 | 173.6438 | 9.10E−01 | 2.70E−01 | 1 | Unchanged Low |
| Q9Y2T2 | 323.8714 | −4.92E−01 | 2.29E−01 | 1 | Unchanged Low |
| Q13105 | 220.1539 | 4.32E−01 | 4.47E−01 | 1 | Unchanged Low |
| Q9H1J1 | 192.8261 | 6.45E−01 | 2.50E−01 | 1 | Unchanged Low |
| Q08752 | 296.8872 | −8.89E−01 | −1.13E+00 | 1 | Unchanged Low |
| P38159 | 253.2584 | −5.07E−01 | −9.58E−01 | 1 | Unchanged Low |
| Q9Y337 | 256.4089 | 1.62E−01 | 5.36E−01 | 1 | Unchanged Low |
| P25445 | 186.0304 | 1.24E+00 | 1.07E+00 | 1 | Unchanged Low |
| Q9BZH1 | 268.2725 | −5.39E−01 | −7.24E−01 | 1 | Unchanged Low |
| O43633 | 260.7188 | −3.53E−01 | −4.10E−01 | 1 | Unchanged Low |
| Q99536 | 288.1564 | 1.17E−01 | 8.44E−01 | 1 | Unchanged Low |
| P24557 | 268.1689 | −4.34E−01 | −4.66E−01 | 1 | Unchanged Low |
| O75369 | 344.1714 | −4.56E−01 | 4.69E−01 | 1 | Unchanged Low |
| P50453 | 350.7941 | −8.60E−01 | −2.49E−01 | 1 | Unchanged Low |
| P35680 | 217.2497 | 4.28E−02 | −4.40E−01 | 1 | Unchanged Low |
| Q03924 | 267.2646 | −8.70E−02 | 2.24E−01 | 1 | Unchanged Low |
| Q9NRQ5 | 309.8867 | −5.76E−01 | −1.56E−01 | 1 | Unchanged Low |
| Q99867 | 289.1694 | −6.29E−01 | −6.08E−01 | 1 | Unchanged Low |
| Q9H682 | 271.5091 | 2.46E−01 | 8.28E−01 | 1 | Unchanged Low |
| O00148 | 214.3036 | −8.71E−02 | −8.92E−01 | 1 | Unchanged Low |
| | 236.7141 | 5.15E−01 | 7.91E−01 | 1 | Unchanged Low |
| Q9UG51 | 283.0875 | −1.69E−01 | 2.70E−01 | 1 | Unchanged Low |
| O75175 | 242.329 | 8.45E−02 | 1.35E−01 | 1 | Unchanged Low |
| Q9GZQ8 | 300.1702 | −1.00E−01 | 5.94E−01 | 1 | Unchanged Low |
| P19438 | 224.9565 | 6.66E−02 | −2.43E−01 | 1 | Unchanged Low |
| Q9NRR8 | 303.9389 | −4.68E−01 | −3.97E−02 | 1 | Unchanged Low |
| O95162 | 169.1352 | 6.60E−01 | −5.27E−01 | 1 | Unchanged Low |
| O95279 | 215.5542 | −1.63E−01 | −1.11E+00 | 1 | Unchanged Low |
| Q9NTW4 | 289.1446 | −7.18E−01 | −8.83E−01 | 1 | Unchanged Low |
| Q9NWM9 | 191.4607 | 1.02E+00 | 7.69E−01 | 1 | Unchanged Low |
| P40121 | 370.7982 | −8.79E−01 | −1.26E−01 | 1 | Unchanged Low |
| Q92466 | 188.2839 | 1.31E+00 | 1.15E+00 | 1 | Unchanged Low |
| O43365 | 191.5647 | 8.91E−01 | 5.60E−01 | 1 | Unchanged Low |
| Q9NVJ7 | 288.7892 | −4.48E−01 | −2.44E−01 | 1 | Unchanged Low |
| Q9NVY5 | 202.4155 | 4.36E−01 | −3.55E−02 | 1 | Unchanged Low |
| Q9BY40 | 173.0132 | 8.07E−01 | −1.06E−01 | 1 | Unchanged Low |
| Q9NZE2 | 152.875 | 1.69E+00 | 8.23E−01 | 1 | Unchanged Low |
| P29275 | 264.0659 | −5.21E−01 | −8.95E−01 | 1 | Unchanged Low |
| Q9NZE0 | 133.8159 | 1.58E+00 | −2.18E−01 | 1 | Unchanged Low |
| Q03111 | 211.7891 | 1.39E−01 | −4.71E−01 | 1 | Unchanged Low |
| Q08499 | 155.5405 | 9.83E−01 | −4.34E−01 | 1 | Unchanged Low |
| Q15628 | 237.3623 | 2.13E−01 | 2.21E−01 | 1 | Unchanged Low |
| Q9Y657 | 222.9201 | 1.89E−01 | −1.07E−01 | 1 | Unchanged Low |
| Q9UHQ1 | 288.0624 | −3.32E−01 | −4.84E−02 | 1 | Unchanged Low |
| P17980 | 257.496 | −4.13E−01 | −7.81E−01 | 1 | Unchanged Low |
| P45877 | 194.3167 | 5.93E−01 | 3.30E−02 | 1 | Unchanged Low |
| O95340 | 149.5355 | 1.17E+00 | −3.26E−01 | 1 | Unchanged Low |
| Q9H9Z7 | 228.8009 | 6.52E−01 | 8.13E−01 | 1 | Unchanged Low |
| O15172 | 287.4495 | −3.68E−01 | −1.45−01 | 1 | Unchanged Low |
| P01298 | 329.1527 | −1.13+00 | −1.49E+00 | 1 | Unchanged Low |
| Q13395 | 248.0711 | −6.41E−02 | −1.55E−01 | 1 | Unchanged Low |
| O15551 | 259.744 | −1.66E−01 | −1.64E−01 | 1 | Unchanged Low |
| P55060 | 282.6907 | −3.68E−01 | −2.21E−01 | 1 | Unchanged Low |
| O43286 | 344.9578 | −3.42E−01 | 5.65E−01 | 1 | Unchanged Low |
| P04424 | 371.2796 | −1.25E+00 | −1.15E+00 | 1 | Unchanged Low |
| Q9NWR5 | 224.6496 | 5.41E−01 | 5.50E−01 | 1 | Unchanged Low |
| Q9BUN8 | 197.0059 | 5.95E−01 | 7.92E−02 | 1 | Unchanged Low |
| P49760 | 138.8925 | 1.85E+00 | 4.88E−01 | 1 | Unchanged Low |
| Q9UEG3 | 284.5589 | −5.53E−01 | −6.42E−01 | 1 | Unchanged Low |
| Q9P0Y6 | 258.2636 | −9.74E−02 | −6.63E−02 | 1 | Unchanged Low |
| Q92535 | 213.6071 | 2.01E−01 | −3.46E−01 | 1 | Unchanged Low |
| Q00577 | 454.6488 | −1.74E+00 | −1.62E+00 | 1 | Unchanged Low |
| O75717 | 242.2724 | 1.52E−01 | 1.44E−01 | 1 | Unchanged Low |
| P04278 | 221.0173 | 2.20E−01 | −1.36E−01 | 1 | Unchanged Low |
| P51124 | 158.1827 | 1.21E+00 | 7.09E−02 | 1 | Unchanged Low |
| P15259 | 315.8301 | −4.05E−01 | 1.22E−01 | 1 | Unchanged Low |
| Q12893 | 235.3565 | 5.32E−01 | 6.82E−01 | 1 | Unchanged Low |
| P57738 | 217.0429 | 5.99E−01 | 4.80E−01 | 1 | Unchanged Low |
| P24863 | 481.4089 | −1.41E+00 | −3.55E−01 | 1 | Unchanged Low |
| Q9Y370 | 187.603 | 7.17E−01 | 2.82E−02 | 1 | Unchanged Low |
| Q9HD89 | 208.2163 | 4.13E−01 | −6.07E−02 | 1 | Unchanged Low |
| O14807 | 179.5238 | 1.27E+00 | 7.81E−01 | 1 | Unchanged Low |
| Q13435 | 263.6077 | −9.65E−02 | −1.45E−02 | 1 | Unchanged Low |
| Q9UMY4 | 219.6698 | 5.00E−01 | 3.45E−01 | 1 | Unchanged Low |
| O43929 | 291.5955 | −7.92E−02 | 4.04E−01 | 1 | Unchanged Low |
| Q9BZQ1 | 278.4192 | 2.07E−01 | 7.21E−01 | 1 | Unchanged Low |
| P55042 | 355.8906 | −2.46E−01 | 7.73E−01 | 1 | Unchanged Low |
| Q92870 | 135.8422 | 1.74E+00 | 3.40E−02 | 1 | Unchanged Low |
| P33778 | 271.5522 | 5.84E−01 | 1.21E+00 | 1 | Unchanged Low |
| Q9ULZ3 | 342.2046 | −1.06E−02 | 1.01E+00 | 1 | Unchanged Low |
| Q13485 | 199.4935 | 6.40E−01 | 1.51E−01 | 1 | Unchanged Low |
| Q9H684 | 280.5396 | −3.61E−01 | −3.26E−01 | 1 | Unchanged Low |
| Q9H7F8 | 234.2579 | 4.22E−01 | 4.40E−01 | 1 | Unchanged Low |
| Q92508 | 237.2652 | 8.06E−02 | −1.62E−01 | 1 | Unchanged Low |
| Q9UM47 | 150.552 | 1.97E+00 | 1.02E+00 | 1 | Unchanged Low |
| P18084 | 214.6124 | −2.33E−02 | −1.00E+00 | 1 | Unchanged Low |
| Q99462 | 213.9424 | 2.98E−01 | −2.15E−01 | 1 | Unchanged Low |
| O75619 | 262.7733 | 3.68E−01 | 7.56E−01 | 1 | Unchanged Low |
| Q92733 | 238.8334 | 3.55E−01 | 3.76E−01 | 1 | Unchanged Low |
| Q15796 | 359.5423 | −5.57E−01 | 2.42E−01 | 1 | Unchanged Low |
| P15291 | 357.1444 | −6.81E−01 | −3.31E−02 | 1 | Unchanged Low |
| Q99417 | 259.037 | −4.00E−02 | −4.48E−02 | 1 | Unchanged Low |
| P06732 | 195.4365 | 3.45E−01 | −6.78E−01 | 1 | Unchanged Low |
| P50570 | 244.4335 | 5.20E−02 | −1.23E−01 | 1 | Unchanged Low |
| Q9Y5A9 | 329.2557 | −4.82E−01 | 4.59E−02 | 1 | Unchanged Low |
| Q92841 | 252.4572 | 1.66E−01 | 2.30E−01 | 1 | Unchanged Low |
| P51808 | 228.2619 | 5.01E−01 | 4.24E−01 | 1 | Unchanged Low |
| O95758 | 377.5392 | −6.74E−01 | 1.67E−01 | 1 | Unchanged Low |
| P32970 | 386.0177 | −7.79E−01 | 4.11E−02 | 1 | Unchanged Low |
| O75689 | 259.1653 | 4.32E−01 | 7.71E−01 | 1 | Unchanged Low |
| P55072 | 362.8018 | −8.50E−01 | −3.63E−01 | 1 | Unchanged Low |
| P47813 | 360.7943 | −9.10E−01 | −5.31E−01 | 1 | Unchanged Low |
| Q15532 | 220.5805 | 4.41E−01 | 1.50E−01 | 1 | Unchanged Low |
| Q15813 | 192.1296 | 7.78E−01 | 1.33E−01 | 1 | Unchanged Low |
| P14373 | 270.5765 | −1.24E−01 | −6.68E−02 | 1 | Unchanged Low |
| O14980 | 235.5264 | 1.17E−01 | −2.08E−01 | 1 | Unchanged Low |
| P07942 | 141.0764 | 1.87E+00 | 3.80E−01 | 1 | Unchanged Low |
| P53041 | 242.7533 | −6.88E−02 | −4.76E−01 | 1 | Unchanged Low |
| Q07820 | 288.4688 | −4.79E−01 | −5.78E−01 | 1 | Unchanged Low |
| P53816 | 474.8201 | −1.22E+00 | −1.19E−01 | 1 | Unchanged Low |
| Q9P1C8 | 234.8631 | 1.23E−01 | −2.19E−01 | 1 | Unchanged Low |
| P51966 | 217.1092 | 8.11E−01 | 7.10E−01 | 1 | Unchanged Low |
| Q99739 | 237.5556 | 5.44E−01 | 6.14E−01 | 1 | Unchanged Low |
| P35913 | 210.8879 | 3.86E−01 | −2.07E−01 | 1 | Unchanged Low |
| P52756 | 257.2441 | −1.04E−01 | −2.87E−01 | 1 | Unchanged Low |
| Q9NXX5 | 196.0576 | 1.01E+00 | 5.96E−01 | 1 | Unchanged Low |
| Q14498 | 224.4664 | 4.16E−01 | 1.38E−01 | 1 | Unchanged Low |
| P28069 | 243.064 | 3.28E−03 | −3.41E−01 | 1 | Unchanged Low |
| P51397 | 307.4499 | −4.10E−01 | −1.57E−01 | 1 | Unchanged Low |
| Q9NUI8 | 249.4637 | 7.36E−01 | 1.06E+00 | 1 | Unchanged Low |
| O00322 | 260.1175 | 2.30E−01 | 3.99E−01 | 1 | Unchanged Low |
| P78368 | 248.257 | 3.12E−01 | 3.63E−01 | 1 | Unchanged Low |
| Q01081 | 163.878 | 9.24E−01 | −5.98E−01 | 1 | Unchanged Low |
| O43193 | 179.1305 | 1.91E+00 | 1.56E+00 | 1 | Unchanged Low |
| Q9Y3B0 | 204.6742 | 5.89E−01 | 2.03E−02 | 1 | Unchanged Low |
| Q9NV80 | 239.9609 | 5.57E−01 | 6.43E−01 | 1 | Unchanged Low |
| Q9Y473 | 226.3406 | 8.96E−01 | 9.57E−01 | 1 | Unchanged Low |
| Q9Y548 | 245.7773 | 1.74E−01 | 4.14E−02 | 1 | Unchanged Low |
| Q13952 | 286.4173 | 1.25E−01 | 5.50E−01 | 1 | Unchanged Low |
| P18615 | 247.7362 | 1.54E−01 | 3.25E−02 | 1 | Unchanged Low |
| P33241 | 163.4955 | 1.48E+00 | 4.71E−01 | 1 | Unchanged Low |
| O15116 | 362.8572 | −3.81E−01 | 4.74E−01 | 1 | Unchanged Low |
| P50897 | 177.9416 | 1.06E+00 | 1.69E−01 | 1 | Unchanged Low |
| | 209.3298 | 9.77E−01 | 7.63E−01 | 1 | Unchanged Low |
| Q9Y3A5 | 426.2946 | −8.25E−01 | 2.14E−01 | 1 | Unchanged Low |
| Q9BVT1 | 291.9599 | −8.95E−02 | 2.14E−01 | 1 | Unchanged Low |
| Q9UM44 | 188.5666 | 5.55E−01 | −5.98E−01 | 1 | Unchanged Low |
| Q9UGC7 | 306.0797 | −1.26E−01 | 3.22E−01 | 1 | Unchanged Low |
| P49146 | 181.9126 | 2.60E−01 | −1.94E+00 | 1 | Unchanged Low |
| P54687 | 154.4299 | 1.36E+00 | −1.41E−01 | 1 | Unchanged Low |
| O60884 | 321.0577 | −3.12E−01 | 1.51E−01 | 1 | Unchanged Low |
| Q9NVC6 | 199.8392 | 5.40E−01 | −2.84E−01 | 1 | Unchanged Low |
| O43399 | 299.1558 | −9.06E−02 | 2.95E−01 | 1 | Unchanged Low |
| O75880 | 179.8238 | 7.65E−01 | −4.28E−01 | 1 | Unchanged Low |
| P49406 | 398.6973 | −1.12E+00 | −7.10E−01 | 1 | Unchanged Low |
| Q9NYK5 | 396.3322 | −8.27E−01 | −7.61E−02 | 1 | Unchanged Low |
| O15428 | 233.6377 | 5.29E−01 | 4.25E−01 | 1 | Unchanged Low |
| O14842 | 219.8563 | 2.65E−01 | −3.93E−01 | 1 | Unchanged Low |
| O15262 | 304.1323 | 1.33E−01 | 7.18E−01 | 1 | Unchanged Low |
| Q9NP61 | 262.4544 | −3.66E−01 | −9.76E−01 | 1 | Unchanged Low |
| O15491 | 292.4461 | 7.23E−02 | 4.80E−01 | 1 | Unchanged Low |
| O75390 | 412.1526 | −1.29E+00 | −1.07E+00 | 1 | Unchanged Low |
| P57088 | 331.588 | −4.93E−01 | −1.13E−01 | 1 | Unchanged Low |
| Q9NRY6 | 291.4542 | −2.06E−02 | 2.96E−01 | 1 | Unchanged Low |
| O15403 | 242.0422 | −5.18E−01 | −2.39E+00 | 1 | Unchanged Low |
| Q9BPZ5 | 227.363 | 1.08E+00 | 1.17E+00 | 1 | Unchanged Low |
| P10301 | 262.4726 | −2.26E−01 | −6.24E−01 | 1 | Unchanged Low |
| | 223.8166 | 6.79E−01 | 4.79E−01 | 1 | Unchanged Low |
| Q9P018 | 270.6564 | 4.58E−01 | 8.20E−01 | 1 | Unchanged Low |
| Q9Y4H2 | 298.8227 | −1.35E−01 | 1.47E−01 | 1 | Unchanged Low |
| Q9UJX3 | 228.4449 | 3.64E−01 | −4.10E−02 | 1 | Unchanged Low |
| Q9H5X0 | 304.1289 | −3.01E−01 | −1.15E−01 | 1 | Unchanged Low |
| Q92820 | 150.359 | 1.80E+00 | 3.70E−01 | 1 | Unchanged Low |
| Q9UKK3 | 352.9975 | −3.10E−01 | 4.15E−01 | 1 | Unchanged Low |
| Q9NWC8 | 296.1206 | −2.15E−01 | −7.10E−02 | 1 | Unchanged Low |
| O14965 | 170.6789 | 9.67E−01 | −4.69E−01 | 1 | Unchanged Low |
| P50613 | 327.4234 | −7.56E−02 | 5.61E−01 | 1 | Unchanged Low |
| Q9H1C4 | 374.1085 | −3.57E−01 | 5.22E−01 | 1 | Unchanged Low |
| P21851 | 328.1045 | −3.14E−01 | 1.39E−01 | 1 | Unchanged Low |
| Q9UGJ0 | 413.4782 | −6.42E−01 | 3.51E−01 | 1 | Unchanged Low |
| P04035 | 228.5315 | 1.70E−01 | −5.20E−01 | 1 | Unchanged Low |
| Q9UKY7 | 396.2551 | −1.23E+00 | −1.24E+00 | 1 | Unchanged Low |
| O95478 | 247.5607 | 1.42E−01 | −1.70E−01 | 1 | Unchanged Low |
| Q9Y3W9 | 275.7642 | 3.15E−01 | 5.94E−01 | 1 | Unchanged Low |
| Q9Y325 | 319.4542 | −3.38E−01 | −3.85E−02 | 1 | Unchanged Low |
| Q92854 | 266.8232 | −3.20E−01 | −9.02E−01 | 1 | Unchanged Low |
| P16106 | 200.5879 | 1.38E+00 | 1.07E+00 | 1 | Unchanged Low |
| O43143 | 345.6042 | −5.86E−01 | −2.41E−01 | 1 | Unchanged Low |
| Q13547 | 340.9663 | −4.48E−01 | −1.34E−02 | 1 | Unchanged Low |
| Q9H6F9 | 290.6336 | −1.55E−01 | −8.00E−02 | 1 | Unchanged Low |
| O9NX18 | 327.1007 | −1.77E−01 | 3.38E−01 | 1 | Unchanged Low |
| O94764 | 266.5947 | 1.06E−01 | 6.24E−02 | 1 | Unchanged Low |
| P82909 | 293.6016 | 7.73E−02 | 3.94E−01 | 1 | Unchanged Low |
| Q9UI07 | 225.0966 | 4.66E−01 | −4.00E−03 | 1 | Unchanged Low |
| P20337 | 205.8132 | 3.62E−01 | −7.65E−01 | 1 | Unchanged Low |
| Q07960 | 237.9303 | 3.28E−01 | −1.55E−02 | 1 | Unchanged Low |
| P49448 | 283.2713 | −3.18E−01 | −5.88E−01 | 1 | Unchanged Low |
| Q9NX48 | 457.1855 | −8.35E−01 | 2.83E−01 | 1 | Unchanged Low |
| Q9NV56 | 173.6478 | 9.98E−01 | −4.11E−01 | 1 | Unchanged Low |
| O00154 | 213.4547 | 6.77E−01 | 1.25E−01 | 1 | Unchanged Low |
| Q9UI70 | 311.4328 | −1.49E−01 | 1.81E−01 | 1 | Unchanged Low |
| P26045 | 334.8518 | −5.70E−01 | −3.90E−01 | 1 | Unchanged Low |
| Q9Y2A8 | 147.0989 | 1.94E+00 | 3.38E−01 | 1 | Unchanged Low |
| Q9BZ89 | 223.0558 | 6.64E−01 | 2.99E−01 | 1 | Unchanged Low |
| P35052 | 221.7567 | 3.76E−01 | −3.13E−01 | 1 | Unchanged Low |
| Q9H0S9 | 171.2074 | 1.80E+00 | 9.51E−01 | 1 | Unchanged Low |
| Q15654 | 260.4802 | 7.71E−02 | −1.49E−01 | 1 | Unchanged Low |
| P51948 | 263.2799 | 2.22E−01 | 1.84E−01 | 1 | Unchanged Low |
| Q13133 | 272.0232 | 1.34E−02 | −8.70E−02 | 1 | Unchanged Low |
| Q9P0J9 | 277.1331 | −2.71E−01 | −6.53E−01 | 1 | Unchanged Low |
| Q15546 | 170.0401 | 1.03E+00 | −5.39E−01 | 1 | Unchanged Low |
| P28325 | 320.2206 | −4.10E−01 | −2.65E−01 | 1 | Unchanged Low |
| O94985 | 207.2587 | 1.03E+00 | 5.80E−01 | 1 | Unchanged Low |
| P48634 | 241.9692 | 7.23E−02 | −5.80E−01 | 1 | Unchanged Low |
| Q9BRL6 | 268.4079 | 3.48E−01 | 4.65E−01 | 1 | Unchanged Low |
| O94851 | 187.2889 | 1.37E+00 | 6.64E−01 | 1 | Unchanged Low |
| Q9NW56 | 278.4551 | 2.25E−01 | 3.85E−01 | 1 | Unchanged Low |
| Q9H6X6 | 255.4932 | 6.17E−01 | 7.19E−01 | 1 | Unchanged Low |
| P41217 | 257.5072 | −2.09E−01 | −9.70E−01 | 1 | Unchanged Low |
| P31512 | 249.9252 | 5.50E−01 | 5.19E−01 | 1 | Unchanged Low |
| Q15425 | 206.4709 | 5.98E−01 | −3.01E−01 | 1 | Unchanged Low |
| P40306 | 400.975 | −5.34E−01 | 3.28E−01 | 1 | Unchanged Low |
| Q9NWZ8 | 238.3826 | −2.85E−02 | −9.82E−01 | 1 | Unchanged Low |
| P57073 | 251.002 | 4.77E−01 | 4.06E−01 | 1 | Unchanged Low |
| Q9Y450 | 274.9973 | 2.91E−01 | 4.34E−01 | 1 | Unchanged Low |
| P04062 | 322.2918 | −6.43E−02 | 3.95E−01 | 1 | Unchanged Low |
| O75065 | 201.6823 | 7.50E−01 | −1.22E−01 | 1 | Unchanged Low |
| Q9ULX2 | 259.9871 | −2.72E−02 | −4.71E−01 | 1 | Unchanged Low |
| Q99871 | 237.9086 | 4.62E−01 | 1.29E−01 | 1 | Unchanged Low |
| Q93065 | 345.3524 | −6.12E−02 | 6.28E−01 | 1 | Unchanged Low |
| O43837 | 310.0846 | −2.99E−01 | −2.30E−01 | 1 | Unchanged Low |
| Q9H9L3 | 257.4051 | 4.37E−01 | 4.12E−01 | 1 | Unchanged Low |
| Q13740 | 256.0277 | 2.94E−01 | 1.28E−01 | 1 | Unchanged Low |
| Q12888 | 213.3434 | 1.09E+00 | 7.48E−01 | 1 | Unchanged Low |
| P78318 | 406.4772 | −5.78E−01 | 2.71E−01 | 1 | Unchanged Low |
| P48436 | 275.3736 | −2.81E−01 | −8.10E−01 | 1 | Unchanged Low |
| P18065 | 284.4952 | −1.22E−02 | −2.05E−02 | 1 | Unchanged Low |
| Q9ULW6 | 218.587 | 3.52E−01 | −5.92E−01 | 1 | Unchanged Low |
| O95154 | 294.419 | −7.41E−02 | −7.00E−03 | 1 | Unchanged Low |
| O95169 | 286.0758 | −3.93E−01 | −9.12E−01 | 1 | Unchanged Low |
| Q99982 | 154.8111 | 2.18E+00 | 8.82E−01 | 1 | Unchanged Low |
| Q99470 | 277.1416 | 3.08E−01 | 4.56E−01 | 1 | Unchanged Low |
| P16104 | 238.3532 | 2.80E−01 | −2.68E−01 | 1 | Unchanged Low |
| Q9UG25 | 298.9147 | 4.21E−01 | 9.00E−01 | 1 | Unchanged Low |
| Q13158 | 276.784 | 1.21E−01 | 9.74E−02 | 1 | Unchanged Low |
| Q9NW32 | 302.6214 | 2.85E−01 | 7.17E−01 | 1 | Unchanged Low |
| O95153 | 224.5106 | 8.88E−01 | 5.75E−01 | 1 | Unchanged Low |
| Q9BQ73 | 265.3817 | 4.37E−01 | 4.85E−01 | 1 | Unchanged Low |
| Q15426 | 341.7927 | −4.79E−02 | 5.71E−01 | 1 | Unchanged Low |
| Q92858 | 392.3029 | −1.09E+00 | −1.14E+00 | 1 | Unchanged Low |
| Q16849 | 162.9842 | 9.77E−01 | −1.39E+00 | 1 | Unchanged Low |
| P11279 | 322.5735 | −3.64E−01 | −2.54E−01 | 1 | Unchanged Low |
| Q96II1 | 157.2667 | 1.47E+00 | −3.63E−01 | 1 | Unchanged Low |
| P78504 | 296.4108 | 1.72E−01 | 4.43E−01 | 1 | Unchanged Low |
| O75937 | 282.0586 | −3.71E−02 | −1.62E−01 | 1 | Unchanged Low |
| O60575 | 194.1988 | 1.02E+00 | 1.00E−01 | 1 | Unchanged Low |
| Q9NYP7 | 257.9759 | −5.42E−02 | −6.81E−01 | 1 | Unchanged Low |
| O95436 | 317.3784 | 2.73E−01 | 8.34E−01 | 1 | Unchanged Low |
| O95324 | 277.0901 | 1.08E−01 | 3.94E−02 | 1 | Unchanged Low |
| Q9UQF6 | 410.9115 | −8.47E−01 | −2.89E−01 | 1 | Unchanged Low |
| P13010 | 239.8403 | 7.26E−01 | 5.51E−01 | 1 | Unchanged Low |
| Q9UJZ1 | 350.5499 | −7.63E−01 | −8.52E−01 | 1 | Unchanged Low |
| P50281 | 204.0657 | 1.19E+00 | 6.26E−01 | 1 | Unchanged Low |
| P15927 | 194.7022 | 1.54E+00 | 9.54E−01 | 1 | Unchanged Low |
| O75496 | 403.7854 | −6.82E−01 | −3.84E−02 | 1 | Unchanged Low |
| Q9P1E3 | 262.3787 | 3.25E−01 | 1.79E−01 | 1 | Unchanged Low |
| P40189 | 312.5572 | −1.84E−01 | −6.89E−02 | 1 | Unchanged Low |
| Q9Y259 | 331.557 | −1.72E−01 | 1.93E−01 | 1 | Unchanged Low |
| O00633 | 226.7943 | 5.46E−01 | −7.94E−02 | 1 | Unchanged Low |
| Q13432 | 229.0999 | 1.20E+00 | 1.08E+00 | 1 | Unchanged Low |
| Q9Y677 | 227.7917 | 6.43E−01 | 1.29E−01 | 1 | Unchanged Low |
| Q9BW24 | 234.5628 | 7.83E−01 | 4.97E−01 | 1 | Unchanged Low |
| Q9NSD9 | 246.1288 | 3.71E−01 | −5.22E−02 | 1 | Unchanged Low |
| Q9NWQ0 | 482.328 | −7.63E−01 | 4.00E−01 | 1 | Unchanged Low |
| Q9NWT6 | 363.8746 | −8.29E−01 | −9.18E−01 | 1 | Unchanged Low |
| Q9UPN3 | 478.6344 | −9.75E−01 | −3.78E−02 | 1 | Unchanged Low |
| Q96HW0 | 258.7634 | 1.22E+00 | 1.49E+00 | 1 | Unchanged Low |
| Q14257 | 249.8842 | 3.11E−01 | −1.36E−01 | 1 | Unchanged Low |
| Q9NRJ5 | 316.91 | −3.59E−01 | −4.60E−01 | 1 | Unchanged Low |
| P23280 | 178.6789 | 1.11E+00 | −4.28E−01 | 1 | Unchanged Low |
| Q14677 | 277.0019 | −1.59E−01 | −6.96E−01 | 1 | Unchanged Low |
| Q9Y394 | 375.1673 | −6.67E−01 | −3.97E−01 | 1 | Unchanged Low |
| Q9UKX5 | 173.978 | 1.58E+00 | 3.42E−01 | 1 | Unchanged Low |
| Q92539 | 264.3623 | 1.88E−01 | −1.49E−01 | 1 | Unchanged Low |
| Q9NW01 | 199.1936 | 8.43E−01 | −2.97E−01 | 1 | Unchanged Low |
| Q9NUW5 | 257.4882 | 3.82E−01 | 1.11E−01 | 1 | Unchanged Low |
| Q9BYC4 | 394.9285 | −6.86E−01 | −2.31E−01 | 1 | Unchanged Low |
| P02545 | 433.306 | −1.25E+00 | −1.27E+00 | 1 | Unchanged Low |
| O60585 | 315.9302 | −9.45E−02 | 5.68E−02 | 1 | Unchanged Low |
| P23458 | 375.1408 | −3.71E−01 | 1.95E−01 | 1 | Unchanged Low |
| Q9BY13 | 401.4116 | −5.44E−01 | 1.14E−01 | 1 | Unchanged Low |
| Q9UBC2 | 247.3586 | 7.96E−01 | 6.59E−01 | 1 | Unchanged Low |
| O76022 | 266.0871 | 2.17E−01 | −8.34E−02 | 1 | Unchanged Low |
| P33993 | 234.4011 | 3.22E−01 | −5.33E−01 | 1 | Unchanged Low |
| Q92963 | 415.4004 | −5.76E−01 | 1.58E−01 | 1 | Unchanged Low |
| Q9HAF1 | 328.0993 | 1.50E−01 | 6.13E−01 | 1 | Unchanged Low |
| Q9NZ36 | 379.485 | −7.53E−01 | −6.05E−01 | 1 | Unchanged Low |
| Q9BQQ3 | 283.9342 | 5.27E−01 | 7.20E−01 | 1 | Unchanged Low |
| O95214 | 337.2896 | 3.87E−02 | 5.19E−01 | 1 | Unchanged Low |
| Q9Y294 | 237.0749 | 8.54E−01 | 5.47E−01 | 1 | Unchanged Low |
| O95810 | 284.3868 | 1.06E−01 | −4.11E−02 | 1 | Unchanged Low |
| P30670 | 412.6188 | −6.41E−01 | −1.59E−02 | 1 | Unchanged Low |
| P51946 | 242.2819 | 7.43E−01 | 4.38E−01 | 1 | Unchanged Low |
| Q15290 | 315.4543 | 1.74E−02 | 2.12E−01 | 1 | Unchanged Low |
| Q9NX11 | 219.2418 | 8.69E−01 | 2.02E−01 | 1 | Unchanged Low |
| Q9P0S3 | 239.5316 | 6.91E−01 | 2.90E−01 | 1 | Unchanged Low |
| P04554 | 352.4183 | −3.79E−01 | −1.20E−01 | 1 | Unchanged Low |
| O15243 | 340.9139 | −1.22E−01 | 2.47E−01 | 1 | Unchanged Low |
| O43717 | 317.004 | 9.07E−02 | 3.54E−01 | 1 | Unchanged Low |
| O95876 | 233.8891 | 8.11E−01 | 3.88E−01 | 1 | Unchanged Low |
| Q9H8D5 | 246.7925 | 2.96E−01 | −3.81E−01 | 1 | Unchanged Low |
| Q9Y3B6 | 361.6747 | −7.01E−01 | −7.80E−01 | 1 | Unchanged Low |
| Q9P0S7 | 195.3392 | 1.09E+00 | −4.11E−02 | 1 | Unchanged Low |
| O60630 | 278.4627 | −3.14E−02 | −4.93E−01 | 1 | Unchanged Low |
| O95285 | 349.7511 | −1.28E−01 | 3.14E−01 | 1 | Unchanged Low |
| Q08945 | 289.639 | −1.37E−01 | −5.48E−01 | 1 | Unchanged Low |
| O43583 | 253.5145 | 5.86E−01 | 3.17E−01 | 1 | Unchanged Low |
| O95149 | 248.5641 | 7.05E−01 | 4.41E−01 | 1 | Unchanged Low |
| Q9HAS8 | 293.0254 | 3.26E−01 | 4.44E−01 | 1 | Unchanged Low |
| Q9BQ39 | 233.2592 | 3.82E−01 | −5.49E−01 | 1 | Unchanged Low |
| O15304 | 182.3257 | 1.78E+00 | 7.83E−01 | 1 | Unchanged Low |
| Q15394 | 345.6465 | −1.88E−01 | 1.42E−01 | 1 | Unchanged Low |
| Q9NZA3 | 284.2776 | 3.79E−01 | 4.07E−01 | 1 | Unchanged Low |
| P26022 | 154.9189 | 1.65E+00 | −6.72E−01 | 1 | Unchanged Low |
| Q00536 | 318.5577 | 7.68E−02 | 3.09E−01 | 1 | Unchanged Low |
| Q9UMW5 | 197.6582 | 2.55E+00 | 2.16E+00 | 1 | Unchanged Low |
| O95353 | 203.4709 | 1.68E+00 | 1.12E+00 | 1 | Unchanged Low |
| O95808 | 240.0311 | 7.11E−01 | 2.60E−01 | 1 | Unchanged Low |
| Q9BXJ8 | 455.5065 | −4.18E−01 | 6.50E−01 | 1 | Unchanged Low |
| P36776 | 354.6324 | −3.40E−01 | −9.78E−02 | 1 | Unchanged Low |
| Q9BUR9 | 299.1204 | 3.03E−01 | 4.35E−01 | 1 | Unchanged Low |
| Q9Y3R1 | 224.7776 | 9.58E−01 | 3.78E−01 | 1 | Unchanged Low |
| Q92997 | 284.6254 | 3.65E−01 | 3.43E−01 | 1 | Unchanged Low |
| O14682 | 274.7193 | −7.50E−02 | −7.90E−01 | 1 | Unchanged Low |
| Q9Y255 | 432.9739 | −7.86E−01 | −2.30E−01 | 1 | Unchanged Low |
| Q16664 | 316.9703 | −1.92E−01 | −3.09E−01 | 1 | Unchanged Low |
| P31943 | 416.9005 | −1.15E+00 | −1.46E+00 | 1 | Unchanged Low |
| P49848 | 198.2917 | 1.04E+00 | −1.87E−01 | 1 | Unchanged Low |
| Q04984 | 325.4422 | −1.20E−01 | −4.81E−02 | 1 | Unchanged Low |
| P20719 | 302.8287 | 1.98E−01 | 2.63E−01 | 1 | Unchanged Low |
| Q9H5Y0 | 313.8457 | −6.25E−02 | −9.24E−02 | 1 | Unchanged Low |
| P19021 | 268.0943 | 5.06E−01 | 3.13E−01 | 1 | Unchanged Low |
| Q92544 | 296.5662 | 2.04E−01 | 1.80E−01 | 1 | Unchanged Low |
| Q12907 | 282.474 | −2.70E−03 | −4.97E−01 | 1 | Unchanged Low |
| Q9BW91 | 324.483 | −1.48E−01 | −1.39E−01 | 1 | Unchanged Low |
| P53365 | 318.382 | 9.37E−02 | 2.51E−01 | 1 | Unchanged Low |
| P35869 | 326.5306 | 3.27E−01 | 7.40E−01 | 1 | Unchanged Low |
| Q13145 | 470.7589 | −6.46E−01 | 3.08E−01 | 1 | Unchanged Low |
| O14747 | 370.593 | −1.51E−01 | 3.69E−01 | 1 | Unchanged Low |
| Q9GZT5 | 334.369 | −2.05E−01 | −1.46E−01 | 1 | Unchanged Low |
| P08174 | 492.3204 | −1.17E+00 | −6.26E−01 | 1 | Unchanged Low |
| Q9H689 | 282.3328 | 3.77E−01 | 2.65E−01 | 1 | Unchanged Low |
| Q9P0T7 | 301.4061 | 2.40E−01 | 2.82E−01 | 1 | Unchanged Low |
| Q9H2V7 | 328.1261 | 1.88E−01 | 5.13E−01 | 1 | Unchanged Low |
| Q9NWT0 | 407.636 | −4.19E−01 | 2.13E−01 | 1 | Unchanged Low |
| Q9Y6X2 | 233.7882 | 1.08E+00 | 6.71E−01 | 1 | Unchanged Low |
| Q9P1P1 | 355.1102 | −4.15E−01 | −3.46E−01 | 1 | Unchanged Low |
| P23919 | 174.3854 | 1.35E+00 | −5.53E−01 | 1 | Unchanged Low |
| P37140 | 391.3748 | −1.69E−01 | 5.03E−01 | 1 | Unchanged Low |
| Q9NW54 | 472.8474 | −4.53E−01 | 6.33E−01 | 1 | Unchanged Low |
| Q9UMS0 | 400.6081 | −6.74E−01 | −4.00E−01 | 1 | Unchanged Low |
| P14207 | 235.1893 | 4.24E−01 | −6.13E−01 | 1 | Unchanged Low |
| Q9UHC7 | 267.5313 | 3.76E−01 | −7.36E−03 | 1 | Unchanged Low |
| O75844 | 276.2357 | −9.63E−02 | −9.58E−01 | 1 | Unchanged Low |
| | 162.8523 | 1.86E+00 | −3.90E−02 | 1 | Unchanged Low |
| P16587 | 369.9292 | −4.86E−01 | −3.52E−01 | 1 | Unchanged Low |
| Q9NVY2 | 378.7795 | −4.06E−01 | −7.72E−02 | 1 | Unchanged Low |
| Q9Y3I1 | 369.3121 | −2.15E−01 | 1.93E−01 | 1 | Unchanged Low |
| Q9H320 | 271.2531 | 1.11E+00 | 1.24E+00 | 1 | Unchanged Low |
| P49903 | 364.5082 | −4.50E−02 | 4.37E−01 | 1 | Unchanged Low |
| O00506 | 289.4017 | 2.22E−01 | 7.86E−03 | 1 | Unchanged Low |
| O00273 | 350.0198 | −4.16E−02 | 2.87E−01 | 1 | Unchanged Low |
| Q08170 | 247.9554 | 4.68E−01 | −2.61E−01 | 1 | Unchanged Low |
| Q9Y3B8 | 240.8904 | 1.41E+00 | 1.22E+00 | 1 | Unchanged Low |
| Q9P0N0 | 362.9626 | −2.38E−01 | 5.03E−02 | 1 | Unchanged Low |
| Q9BRI3 | 361.6895 | −2.21E−01 | 6.60E−02 | 1 | Unchanged Low |
| Q9Y319 | 183.4471 | 1.58E+00 | 1.97E−01 | 1 | Unchanged Low |
| Q16514 | 246.9777 | 1.46E+00 | 1.38E+00 | 1 | Unchanged Low |
| P13489 | 259.1076 | 5.18E−01 | 5.00E−02 | 1 | Unchanged Low |
| Q99987 | 240.3908 | 6.42E−01 | −8.59E−02 | 1 | Unchanged Low |
| P56181 | 358.4382 | −4.37E−01 | −4.48E−01 | 1 | Unchanged Low |
| P51148 | 336.6203 | −3.64E−02 | 1.20E−01 | 1 | Unchanged Low |
| Q9HD34 | 276.3612 | 3.22E−01 | −5.12E−02 | 1 | Unchanged Low |
| Q9HAN2 | 313.4126 | 1.43E−01 | 1.58E−01 | 1 | Unchanged Low |
| P08107 | 413.3803 | −7.31E−01 | −4.87E−01 | 1 | Unchanged Low |
| O60499 | 272.2579 | 7.66E−01 | 6.81E−01 | 1 | Unchanged Low |
| Q9HBL3 | 237.4707 | 9.68E−01 | 4.38E−01 | 1 | Unchanged Low |
| Q9BSJB | 482.4222 | −7.63E−01 | 6.53E−02 | 1 | Unchanged Low |
| O60838 | 264.8237 | 5.87E−01 | 2.33E−01 | 1 | Unchanged Low |
| Q98TM6 | 343.6429 | −1.97E−01 | −1.53E−01 | 1 | Unchanged Low |
| Q9H4L0 | 147.5387 | 2.78E+00 | 5.77E−01 | 1 | Unchanged Low |
| P50402 | 316.6654 | 1.47E−02 | −8.04E−02 | 1 | Unchanged Low |
| Q9NZP7 | 299.844 | −4.98E−02 | −4.94E−01 | 1 | Unchanged Low |
| P49116 | 281.6482 | 1.36E−01 | −4.03E−01 | 1 | Unchanged Low |
| P11234 | 444.9874 | −7.97E−01 | −3.51E−01 | 1 | Unchanged Low |
| P15408 | 342.8236 | 2.94E−01 | 7.18E−01 | 1 | Unchanged Low |
| Q9P1Q8 | 312.2142 | −1.33E−01 | −5.03E−01 | 1 | Unchanged Low |
| P39060 | 230.0488 | 1.11E+00 | 4.81E−01 | 1 | Unchanged Low |
| Q9Y2N3 | 319.0933 | −2.13E−01 | −5.91E−01 | 1 | Unchanged Low |
| P02749 | 320.758 | −3.21E−01 | −8.52E−01 | 1 | Unchanged Low |
| Q9P189 | 359.0228 | −2.62E−01 | −1.34E−01 | 1 | Unchanged Low |
| O43822 | 291.7148 | 3.91E−01 | 2.50E−01 | 1 | Unchanged Low |
| Q9UNZ2 | 420.0654 | −3.35E−01 | 3.27E−01 | 1 | Unchanged Low |
| Q9Y4G5 | 376.7971 | −2.00E−01 | 1.77E−01 | 1 | Unchanged Low |
| Q13310 | 327.507 | 5.03E−02 | 9.11E−02 | 1 | Unchanged Low |
| P45974 | 304.4284 | 2.95E−01 | 2.39E−01 | 1 | Unchanged Low |
| P55317 | 302.8162 | 2.65E−01 | 1.60E−01 | 1 | Unchanged Low |
| O43364 | 435.9554 | −6.35E−01 | −1.28E−01 | 1 | Unchanged Low |
| P55345 | 333.8774 | −1.47E−01 | −2.48E−01 | 1 | Unchanged Low |
| P48230 | 437.5376 | −7.73E−01 | −4.40E−01 | 1 | Unchanged Low |
| Q9BUY3 | 264.7192 | 9.91E−01 | 8.34E−01 | 1 | Unchanged Low |
| O00115 | 326.0947 | 2.66E−01 | 4.34E−01 | 1 | Unchanged Low |
| O43318 | 254.8269 | 8.26E−01 | 4.01E−01 | 1 | Unchanged Low |
| O95572 | 287.175 | 3.91E−01 | 1.33E−01 | 1 | Unchanged Low |
| P35080 | 176.518 | 1.58E+00 | −3.76E−01 | 1 | Unchanged Low |
| Q9NW29 | 241.9521 | 6.00E−01 | −3.40E−01 | 1 | Unchanged Low |
| Q9NZE9 | 299.9861 | 6.01E−01 | 6.64E−01 | 1 | Unchanged Low |
| Q9BYV2 C | 390.6938 | −5.83E−01 | −5.66E−01 | 1 | Unchanged Low |
| Q9UJX4 | 280.1684 | 4.97E−01 | 1.81E−01 | 1 | Unchanged Low |
| O00302 | 347.7981 | 3.31E−02 | 2.22E−01 | 1 | Unchanged Low |
| Q92886 | 302.7027 | 2.07E−01 | −3.76E−02 | 1 | Unchanged Low |
| O14929 | 270.1111 | −4.02E−02 | −1.41E+00 | 1 | Unchanged Low |
| Q9NVK7 | 301.1995 | 2.54E−01 | 2.92E−02 | 1 | Unchanged Low |
| Q9UBN0 | 329.7234 | 1.14E−01 | 1.50E−01 | 1 | Unchanged Low |
| O43611 | 245.5832 | 8.94E−01 | 2.80E−01 | 1 | Unchanged Low |
| O94777 | 291.5641 | 1.95E−01 | −2.71E−01 | 1 | Unchanged Low |
| Q14669 | 406.8782 | −5.31E−01 | −2.96E−01 | 1 | Unchanged Low |
| Q9NUR0 | 168.9127 | 2.42E+00 | 7.56E−01 | 1 | Unchanged Low |
| Q16822 | 251.8272 | 3.61E−01 | −7.52E−01 | 1 | Unchanged Low |
| Q9Y640 | 257.3848 | 6.48E−01 | 2.92E−02 | 1 | Unchanged Low |
| P48595 | 269.7194 | 2.44E+00 | 2.72E+00 | 1 | Unchanged Low |
| P26639 | 371.5003 | −7.20E−02 | 2.47E−01 | 1 | Unchanged Low |
| P29597 | 268.5377 | 6.92E−01 | 3.02E−01 | 1 | Unchanged Low |
| O60671 | 210.3983 | 8.49E−01 | −8.59E−01 | 1 | Unchanged Low |
| Q9NW97 | 379.0815 | 3.71E−01 | 1.03E+00 | 1 | Unchanged Low |
| Q9P027 | 439.5495 | −8.60E−01 | −7.62E−01 | 1 | Unchanged Low |
| O95772 | 254.0574 | 7.93E−01 | 2.12E−01 | 1 | Unchanged Low |
| Q9NR28 | 384.7351 | 3.24E−01 | 1.00E+00 | 1 | Unchanged Low |
| P11413 | 244.4906 | 3.98E−01 | −9.31E−01 | 1 | Unchanged Low |
| Q99567 | 375.0149 | −4.48E−01 | −5.38E−01 | 1 | Unchanged Low |
| Q9BZR9 | 315.1764 | 7.20E−01 | 9.45E−01 | 1 | Unchanged Low |
| O43632 | 330.4468 | 5.81E−02 | −2.73E−02 | 1 | Unchanged Low |
| Q9Y305 | 421.5064 | −3.91E−01 | 8.49E−02 | 1 | Unchanged Low |
| P17096 | 333.78 | −2.04E−01 | −5.62E−01 | 1 | Unchanged Low |
| P28677 | 272.797 | 5.38E−01 | 3.76E−02 | 1 | Unchanged Low |
| Q9UBX5 | 176.4964 | 2.10E+00 | 4.44E−01 | 1 | Unchanged Low |
| Q9Y304 | 301.8585 | 6.87E−02 | −4.56E−01 | 1 | Unchanged Low |
| O15379 | 306.8418 | 7.90E−01 | 9.41E−01 | 1 | Unchanged Low |
| Q02790 | 302.2597 | 8.58E−02 | −4.33E−01 | 1 | Unchanged Low |
| Q9H240 | 393.3189 | 3.44E−01 | 1.06E+00 | 1 | Unchanged Low |
| O76095 | 274.4316 | 4.42E−01 | −1.56E−01 | 1 | Unchanged Low |
| Q9Y5Q8 | 404.6602 | −6.66E−01 | −7.50E−01 | 1 | Unchanged Low |
| Q9NNW5 | 322.3381 | 4.37E−01 | 5.37E−01 | 1 | Unchanged Low |
| P01121 | 296.4811 | 5.09E−01 | 3.29E−01 | 1 | Unchanged Low |
| Q9H0V7 | 405.6684 | −7.23E−01 | −9.00E−01 | 1 | Unchanged Low |
| O60664 | 365.5782 | −5.94E−01 | −1.16E+00 | 1 | Unchanged Low |
| P56554 | 378.959 | −1.18E−01 | 1.57E−01 | 1 | Unchanged Low |
| O00330 | 373.6892 | −2.99E−03 | 3.14E−01 | 1 | Unchanged Low |
| O60784 | 342.086 | −7.76E−02 | −2.02E−01 | 1 | Unchanged Low |
| Q9NQZ0 | 491.0715 | −1.19E+00 | −1.22E+00 | 1 | Unchanged Low |
| P52790 | 345.2169 | 4.07E−01 | 7.21E−01 | 1 | Unchanged Low |
| P52815 | 285.7775 | 5.61E−01 | 2.43E−01 | 1 | Unchanged Low |
| O14983 | 245.4613 | 1.05E+00 | 4.12E−01 | 1 | Unchanged Low |
| P04259 | 428.9832 | −6.20E−01 | −3.99E−01 | 1 | Unchanged Low |
| P16455 | 318.0812 | 2.19E−01 | 3.81E−02 | 1 | Unchanged Low |
| Q9Y5L9 | 273.2945 | 2.87E−01 | −6.13E−01 | 1 | Unchanged Low |
| P41214 | 297.4677 | 5.56E−01 | 3.72E−01 | 1 | Unchanged Low |
| Q9H6U4 | 314.4039 | −2.65E−01 | −1.26E+00 | 1 | Unchanged Low |
| Q15393 | 277.0735 | 5.30E−01 | −1.59E−02 | 1 | Unchanged Low |
| P55055 | 347.7942 | 1.03E−01 | 1.76E−01 | 1 | Unchanged Low |
| P51805 | 345.4093 | 2.10E−01 | 3.36E−01 | 1 | Unchanged Low |
| O75815 | 297.768 | 4.94E−01 | 2.34E−01 | 1 | Unchanged Low |
| Q9Y2Y9 | 319.2905 | 5.42E−01 | 6.00E−01 | 1 | Unchanged Low |
| Q99633 | 368.1886 | −2.05E−01 | −2.18E−01 | 1 | Unchanged Low |
| P05155 | 329.7432 | 2.13E−01 | 1.41E−01 | 1 | Unchanged Low |
| Q9UN50 | 333.1657 | 1.27E−01 | 1.84E−02 | 1 | Unchanged Low |
| Q14011 | 352.0144 | 2.66E−01 | 4.94E−01 | 1 | Unchanged Low |
| O75807 | 458.6852 | −7.67E−02 | 8.10E−01 | 1 | Unchanged Low |
| Q14457 | 466.4393 | −6.18E−02 | 8.80E−01 | 1 | Unchanged Low |
| Q02818 | 298.8382 | 9.39E−02 | −6.39E−01 | 1 | Unchanged Low |
| O15235 | 391.6738 | −7.66E−03 | 3.94E−01 | 1 | Unchanged Low |
| Q9Y3E0 | 275.4907 | 1.21E+00 | 1.07E+00 | 1 | Unchanged Low |
| Q9NQZ5 | 303.5369 | 3.51E−01 | 1.19E−02 | 1 | Unchanged Low |
| Q9UHW4 | 303.9535 | 4.06E−01 | 1.25E−01 | 1 | Unchanged Low |
| P82914 | 405.0567 | −3.25E−01 | −9.27E−02 | 1 | Unchanged Low |
| P25787 | 437.9448 | −2.86E−01 | 2.80E−01 | 1 | Unchanged Low |
| P26447 | 275.5734 | 8.38E−01 | 4.57E−01 | 1 | Unchanged Low |
| Q9Y605 | 373.9359 | −2.11E−01 | −2.10E−01 | 1 | Unchanged Low |
| Q14776 | 276.788 | 8.15E−01 | 4.34E−01 | 1 | Unchanged Low |
| P30837 | 479.1158 | −8.27E−01 | −4.86E−01 | 1 | Unchanged Low |
| P09668 | 460.5856 | −3.69E−01 | 2.92E−01 | 1 | Unchanged Low |
| Q02313 | 383.4797 | −1.73E−01 | −4.02E−02 | 1 | Unchanged Low |
| Q13148 | 344.328 | 1.47E−01 | 1.31E−01 | 1 | Unchanged Low |
| Q16645 | 353.4204 | 7.21E−01 | 1.17E+00 | 1 | Unchanged Low |
| | 168.643 | 2.20E+00 | −1.24E−01 | 1 | Unchanged Low |
| P57739 | 261.2968 | 9.26E−01 | 3.41E−01 | 1 | Unchanged Low |
| O60568 | 319.8117 | 1.05E−01 | −3.04E−01 | 1 | Unchanged Low |
| Q9HAU4 | 482.7167 | −2.51E−01 | 6.44E−01 | 1 | Unchanged Low |
| P49914 | 312.027 | 7.38E−02 | −5.17E−01 | 1 | Unchanged Low |
| O60510 | 265.2168 | 1.06E+00 | 6.26E−01 | 1 | Unchanged Low |
| Q9BX68 | 308.6409 | 7.92E−01 | 7.99E−01 | 1 | Unchanged Low |
| P29084 | 281.687 | 4.57E−01 | −2.17E−01 | 1 | Unchanged Low |
| P09526 | 344.9493 | 1.64E−01 | 1.49E−01 | 1 | Unchanged Low |
| O15471 | 279.8225 | 7.09E−01 | 2.44E−01 | 1 | Unchanged Low |
| Q9UEE9 | 242.684 | 1.54E+00 | 9.79E−01 | 1 | Unchanged Low |
| Q15011 | 298.5074 | 2.94E−02 | −9.43E−01 | 1 | Unchanged Low |
| Q9BWG8 | 244.6389 | 1.08E+00 | 2.57E−01 | 1 | Unchanged Low |
| Q9Y397 | 428.8727 | −2.58E−01 | 1.99E−01 | 1 | Unchanged Low |
| Q14596 | 355.7499 | 2.54E−01 | 4.08E−01 | 1 | Unchanged Low |
| P23497 | 394.8464 | 1.25E−01 | 5.64E−01 | 1 | Unchanged Low |
| Q16633 | 188.1549 | 1.81E+00 | −5.39E−02 | 1 | Unchanged Low |
| Q99961 | 280.235 | 6.51E−01 | 9.47E−02 | 1 | Unchanged Low |
| Q07021 | 345.9018 | −3.77E−01 | −1.22E+00 | 1 | Unchanged Low |
| Q16513 | 490.5792 | −5.83E−01 | 3.97E−02 | 1 | Unchanged Low |
| Q9H070 | 331.9695 | 4.61E−02 | −3.46E−01 | 1 | Unchanged Low |
| Q13227 | 424.6909 | −6.12E−01 | −6.65E−01 | 1 | Unchanged Low |
| P20339 | 478.1731 | −1.46E−01 | 7.15E−01 | 1 | Unchanged Low |
| Q9NXX4 | 255.5867 | 1.24E+00 | 6.61E−01 | 1 | Unchanged Low |
| Q92900 | 336.7297 | 1.58E−01 | −5.69E−02 | 1 | Unchanged Low |
| Q9H2K4 | 240.051 | 8.83E−01 | −3.58E−01 | 1 | Unchanged Low |
| O14917 | 309.398 | 5.50E−01 | 3.13E−01 | 1 | Unchanged Low |
| Q9NZ88 | 309.7781 | 8.79E−01 | 8.60E−01 | 1 | Unchanged Low |
| Q9BYC8 | 351.4981 | 5.13E−01 | 7.33E−01 | 1 | Unchanged Low |
| Q15311 | 358.3107 | 3.19E−01 | 4.76E−01 | 1 | Unchanged Low |
| Q9NWE9 | 402.6031 | −3.02E−01 | −2.50E−01 | 1 | Unchanged Low |
| O95571 | 425.0962 | −1.06E−01 | 3.50E−01 | 1 | Unchanged Low |
| P36941 | 409.094 | −1.23E−01 | 1.72E−01 | 1 | Unchanged Low |
| P49790 | 317.116 | 4.85E−01 | 2.58E−01 | 1 | Unchanged Low |
| Q99607 | 302.1703 | 4.75E−01 | 4.79E−03 | 1 | Unchanged Low |
| Q9UIV1 | 345.9619 | 8.86E−01 | 1.21E+00 | 1 | Unchanged Low |
| Q9Y3E4 | 247.1535 | 1.79E+00 | 1.29E+00 | 1 | Unchanged Low |
| P19367 | 366.4437 | −4.61E−01 | −1.25E+00 | 1 | Unchanged Low |
| Q14250 | 272.9152 | 6.99E−01 | −7.52E−02 | 1 | Unchanged Low |
| Q9UL33 | 450.4535 | 1.18E−01 | 8.90E−01 | 1 | Unchanged Low |
| Q15019 | 378.666 | −3.91E−01 | −8.36E−01 | 1 | Unchanged Low |
| Q13084 | 357.6504 | −1.77E−01 | −6.01E−01 | 1 | Unchanged Low |
| Q9UMR2 | 393.1434 | −4.04E−02 | 1.40E−01 | 1 | Unchanged Low |
| P55855 | 424.0867 | −2.84E−01 | −3.58E−02 | 1 | Unchanged Low |
| Q13155 | 368.4293 | 2.12E−01 | 3.42E−01 | 1 | Unchanged Low |
| P78371 | 428.2475 | −3.62E−01 | −1.60E−01 | 1 | Unchanged Low |
| P08173 | 444.3189 | −2.47E−01 | 2.11E−01 | 1 | Unchanged Low |
| O60478 | 362.4989 | 4.47E−01 | 6.73E−01 | 1 | Unchanged Low |
| O60429 | 258.282 | 1.39E+00 | 8.47E−01 | 1 | Unchanged Low |
| O75935 | 405.4281 | −1.25E−01 | 7.97E−02 | 1 | Unchanged Low |
| Q13147 | 307.9098 | 3.28E−01 | −2.63E−01 | 1 | Unchanged Low |
| Q9NPH4 | 295.4166 | 8.60E−01 | 5.45E−01 | 1 | Unchanged Low |
| Q9UJ96 | 468.9343 | −6.30E−01 | −3.80E−01 | 1 | Unchanged Low |
| P17081 | 362.2816 | 3.61E−01 | 5.09E−01 | 1 | Unchanged Low |
| P11166 | 373.8343 | 9.84E−02 | 1.61E−01 | 1 | Unchanged Low |
| Q14820 | 389.2167 | −2.30E−02 | 9.19E−02 | 1 | Unchanged Low |
| Q92935 | 437.6869 | −8.08E−01 | −1.26E+00 | 1 | Unchanged Low |
| P31749 | 355.1851 | −3.24E−02 | −3.46E−01 | 1 | Unchanged Low |
| Q13454 | 355.5691 | 2.06E−01 | 1.50E−01 | 1 | Unchanged Low |
| P35520 | 172.0203 | 2.41E+00 | −5.72E−02 | 1 | Unchanged Low |
| P53602 | 412.6357 | −8.51E−01 | −1.94E+00 | 1 | Unchanged Low |
| Q9H7G3 | 306.3867 | 1.81E−01 | −6.86E−01 | 1 | Unchanged Low |
| Q13123 | 358.3456 | 5.53E−01 | 7.69E−01 | 1 | Unchanged Low |
| O14744 | 378.8177 | 1.21E−01 | 2.37E−01 | 1 | Unchanged Low |
| Q9Y566 | 290.0718 | 8.59E−01 | 4.27E−01 | 1 | Unchanged Low |
| Q9BUJ9 | 412.4352 | −1.79E−02 | 2.94E−01 | 1 | Unchanged Low |
| P51790 | 315.7621 | 2.04E−01 | −4.80E−01 | 1 | Unchanged Low |
| O14547 | 291.9777 | 6.71E−01 | 1.01E−01 | 1 | Unchanged Low |
| Q99753 | 345.0384 | 5.66E−01 | 6.27E−01 | 1 | Unchanged Low |
| Q9NQC8 | 280.7773 | 1.04E+00 | 5.75E−01 | 1 | Unchanged Low |
| O43919 | 230.823 | 8.83E−01 | −9.82E−01 | 1 | Unchanged Low |
| P78345 | 411.2575 | 2.46E−01 | 7.21E−01 | 1 | Unchanged Low |
| P54257 | 384.9948 | −2.35E−01 | −4.83E−01 | 1 | Unchanged Low |
| Q9Y3F4 | 369.3734 | −3.11E−02 | −2.25E−01 | 1 | Unchanged Low |
| P29536 | 311.936 | 1.57E−01 | −7.13E−01 | 1 | Unchanged Low |
| O75925 | 471.2944 | −1.44E−01 | 5.15E−01 | 1 | Unchanged Low |
| Q9UMX5 | 345.2233 | 1.57E−01 | −1.71E−01 | 1 | Unchanged Low |
| Q9UMZ9 | 337.1554 | 1.42E−01 | −3.28E−01 | 1 | Unchanged Low |
| Q92905 | 345.1985 | 5.84E−01 | 6.06E−01 | 1 | Unchanged Low |
| Q9NSD7 | 466.2798 | −9.26E−01 | −1.40E+00 | 1 | Unchanged Low |
| Q9P2W3 | 179.6321 | 2.45E+00 | 2.04E−01 | 1 | Unchanged Low |
| Q9BVI7 | 424.8293 | 7.34E−02 | 4.99E−01 | 1 | Unchanged Low |
| Q9Y4L1 | 390.9975 | −9.20E−02 | −1.49E−01 | 1 | Unchanged Low |
| Q02363 | 410.6421 | −3.20E−01 | −4.33E−01 | 1 | Unchanged Low |
| Q9Y5J8 | 456.8557 | −1.81E−01 | 2.99E−01 | 1 | Unchanged Low |
| | 446.2248 | −3.57E−01 | −1.50E−01 | 1 | Unchanged Low |
| Q99501 | 429.6515 | 1.15E−01 | 5.84E−01 | 1 | Unchanged Low |
| Q00613 | 376.2292 | 1.01E−01 | 4.71E−02 | 1 | Unchanged Low |
| Q9H5Z1 | 217.0007 | 1.51E+00 | −1.35E−01 | 1 | Unchanged Low |
| P09661 | 315.5701 | −1.77E−01 | −1.94E+00 | 1 | Unchanged Low |
| P54709 | 416.5779 | −1.20E−01 | 2.22E−02 | 1 | Unchanged Low |
| P13164 | 231.1336 | 3.12E+00 | 2.50E+00 | 1 | Unchanged Low |
| Q9Y6E2 | 361.6248 | 2.50E−01 | 1.37E−01 | 1 | Unchanged Low |
| Q9NVG2 | 436.0959 | 1.28E−01 | 6.34E−01 | 1 | Unchanged Low |
| P27448 | 386.4633 | −1.97E−02 | −1.08E−01 | 1 | Unchanged Low |
| O15438 | 499.6643 | −6.54E−01 | −3.40E−01 | 1 | Unchanged Low |
| Q9UL48 | 261.8209 | 1.16E+00 | 2.88E−01 | 1 | Unchanged Low |
| O94763 | 420.3384 | 2.40E−01 | 6.77E−01 | 1 | Unchanged Low |
| Q13190 | 269.5995 | 1.13E+00 | 3.89E−01 | 1 | Unchanged Low |
| Q13122 | 383.7948 | 4.94E−02 | −2.22E−02 | 1 | Unchanged Low |
| O75312 | 388.3905 | 1.79E−01 | 2.74E−01 | 1 | Unchanged Low |
| P37837 | 373.7275 | −1.30E−02 | −2.92E−01 | 1 | Unchanged Low |
| O75762 | 327.3962 | 8.28E−01 | 7.06E−01 | 1 | Unchanged Low |
| Q99969 | 312.0672 | 4.82E−01 | −1.44E−01 | 1 | Unchanged Low |
| Q9UNP2 | 227.7665 | 1.41E+00 | −1.15E−01 | 1 | Unchanged Low |
| P56524 | 342.9334 | 2.73E−01 | −1.17E−01 | 1 | Unchanged Low |
| Q9H644 | 255.3931 | 1.49E+00 | 6.93E−01 | 1 | Unchanged Low |
| Q13627 | 407.7774 | −3.27E−01 | −6.06E−01 | 1 | Unchanged Low |
| Q03405 | 431.3524 | −3.29E−01 | −3.38E−01 | 1 | Unchanged Low |
| Q9NPQ8 | 404.6217 | 9.57E−02 | 2.48E−01 | 1 | Unchanged Low |
| Q15375 | 310.569 | 7.13E−01 | 2.55E−01 | 1 | Unchanged Low |
| P05114 | 291.2748 | 1.02E+00 | 4.99E−01 | 1 | Unchanged Low |
| Q9UKU4 | 422.4244 | 1.07E−01 | 4.23E−01 | 1 | Unchanged Low |
| P08195 | 395.768 | −1.47E−01 | −3.58E−01 | 1 | Unchanged Low |
| Q15545 | 442.5426 | 1.59E−03 | 3.92E−01 | 1 | Unchanged Low |
| | 290.8781 | 7.04E−01 | −1.31E−01 | 1 | Unchanged Low |
| Q05397 | 410.2909 | 4.23E−02 | 1.73E−01 | 1 | Unchanged Low |
| Q9Y6G1 | 439.8467 | −1.15E−01 | 1.53E−01 | 1 | Unchanged Low |
| P41212 | 406.6035 | 3.48E−01 | 6.70E−01 | 1 | Unchanged Low |
| O14737 | 443.7457 | −3.10E−01 | −2.13E−01 | 1 | Unchanged Low |
| Q9UHB9 | 287.2236 | 9.21E−01 | 2.16E−01 | 1 | Unchanged Low |
| Q96D21 | 427.9499 | 2.36E−02 | 2.94E−01 | 1 | Unchanged Low |
| Q9Y4I1 | 375.8247 | 1.15E−01 | −7.23E−02 | 1 | Unchanged Low |
| Q9Y5L4 | 354.9111 | 4.70E−01 | 3.39E−01 | 1 | Unchanged Low |
| O75439 | 367.0789 | −3.42E−01 | −1.48E+00 | 1 | Unchanged Low |
| P55010 | 376.3076 | −7.62E−02 | −5.31E−01 | 1 | Unchanged Low |
| Q9BY44 | 420.1404 | −1.60E−01 | −1.78E−01 | 1 | Unchanged Low |
| Q13164 | 290.5304 | 9.58E−01 | 2.93E−01 | 1 | Unchanged Low |
| P53667 | 386.0829 | 1.77E−01 | 1.26E−01 | 1 | Unchanged Low |
| Q9UNH6 | 427.3762 | −2.38E−01 | −2.78E−01 | 1 | Unchanged Low |
| O00258 | 415.5431 | 2.63E−01 | 5.60E−01 | 1 | Unchanged Low |
| Q9Y3I0 | 470.3717 | −1.70E−01 | 2.52E−01 | 1 | Unchanged Low |
| Q9Y368 | 226.9908 | 1.83E+00 | 4.80E−01 | 1 | Unchanged Low |
| P14866 | 374.6383 | −1.19E−01 | −6.92E−01 | 1 | Unchanged Low |
| Q13885 | 420.8456 | −1.14E−01 | −9.41E−02 | 1 | Unchanged Low |
| O43765 | 408.6939 | 1.09E−01 | 2.10E−01 | 1 | Unchanged Low |
| O14632 | 446.1081 | 5.18E−02 | 4.40E−01 | 1 | Unchanged Low |
| P31939 | 432.286 | −2.92E−01 | −3.76E−01 | 1 | Unchanged Low |
| P28331 | 438.7901 | −2.61E−01 | −2.35E−01 | 1 | Unchanged Low |
| P41970 | 325.3706 | 9.96E−01 | 8.03E−01 | 1 | Unchanged Low |
| Q92973 | 346.1733 | 6.20E−02 | −7.51E−01 | 1 | Unchanged Low |
| P36507 | 414.6953 | 5.79E−02 | 1.46E−01 | 1 | Unchanged Low |
| Q16512 | 451.4528 | −1.48E−01 | 9.18E−02 | 1 | Unchanged Low |
| O43447 | 271.0162 | 1.20E+00 | 3.05E−01 | 1 | Unchanged Low |
| P30040 | 351.5665 | 2.50E−01 | −2.29E−01 | 1 | Unchanged Low |
| O95399 | 410.4914 | −1.30E−01 | −3.04E−01 | 1 | Unchanged Low |
| | 401.0113 | −2.74E−01 | −7.87E−01 | 1 | Unchanged Low |
| Q12792 | 418.007 | −2.05E−01 | −3.87E−01 | 1 | Unchanged Low |
| Q14318 | 435.6453 | −1.58E−01 | −9.35E−02 | 1 | Unchanged Low |
| O75955 | 343.496 | 4.47E−01 | 2.68E−02 | 1 | Unchanged Low |
| Q9BVY8 | 440.9559 | −2.37E−01 | −2.35E−01 | 1 | Unchanged Low |
| Q16288 | 243.0358 | 1.72E+00 | 5.53E−01 | 1 | Unchanged Low |
| O75380 | 426.4999 | −1.10E−01 | −1.21E−01 | 1 | Unchanged Low |
| Q9BY51 | 400.0464 | −4.04E−02 | −2.80E−01 | 1 | Unchanged Low |
| Q9BVK3 | 422.5145 | −1.47E−01 | −2.56E−01 | 1 | Unchanged Low |
| Q9P0N3 | 281.424 | 1.60E+00 | 1.07E+00 | 1 | Unchanged Low |
| P24534 | 364.5026 | 2.68E−01 | −8.11E−02 | 1 | Unchanged Low |
| P49336 | 375.2575 | −2.82E−02 | −6.00E−01 | 1 | Unchanged Low |
| P34130 | 323.0643 | 3.54E−01 | −5.53E−01 | 1 | Unchanged Low |
| O75425 | 366.1692 | 3.01E−01 | −7.34E−03 | 1 | Unchanged Low |
| Q15293 | 304.1243 | 7.99E−01 | 5.01E−02 | 1 | Unchanged Low |
| O95376 | 332.2465 | 7.28E−01 | 3.41E−01 | 1 | Unchanged Low |
| O75489 | 375.1108 | 1.69E−01 | −1.99E−01 | 1 | Unchanged Low |
| P28482 | 456.1064 | −2.25E−01 | −1.28E−01 | 1 | Unchanged Low |
| P20823 | 424.3654 | 3.93E−01 | 7.09E−01 | 1 | Unchanged Low |
| O15250 | 496.7532 | −3.09E−01 | 4.57E−02 | 1 | Unchanged Low |
| P55769 | 346.68 | 1.63E+00 | 1.80E+00 | 1 | Unchanged Low |
| O94817 | 485.9574 | −5.74E−01 | −8.52E−01 | 1 | Unchanged Low |
| O75352 | 406.7998 | 6.41E−01 | 9.41E−01 | 1 | Unchanged Low |
| Q16585 | 324.9166 | 7.31E−01 | 1.96E−01 | 1 | Unchanged Low |
| Q9H7X7 | 383.5805 | 6.99E−01 | 8.23E−01 | 1 | Unchanged Low |
| O15173 | 321.6602 | 5.29E−01 | −2.74E−01 | 1 | Unchanged Low |
| Q9GZX9 | 219.3062 | 2.33E+00 | 7.99E−01 | 1 | Unchanged Low |
| Q16594 | 372.6331 | 1.32E+00 | 1.60E+00 | 1 | Unchanged Low |
| O00479 | 348.9168 | 6.84E−01 | 4.15E−01 | 1 | Unchanged Low |
| P54760 | 456.4179 | −3.08E−01 | −3.55E−01 | 1 | Unchanged Low |
| P25490 | 479.4548 | −1.72E−01 | 1.39E−01 | 1 | Unchanged Low |
| Q9UJY0 | 454.8441 | −2.44E−01 | −2.31E−01 | 1 | Unchanged Low |
| Q09666 | 406.041 | 1.24E−01 | 2.41E−02 | 1 | Unchanged Low |
| O15305 | 411.2528 | 3.98E−01 | 5.63E−01 | 1 | Unchanged Low |
| O60492 | 343.3316 | 1.42E−01 | −8.68E−01 | 1 | Unchanged Low |
| Q9UND3 | 340.6147 | 6.10E−01 | 1.54E−01 | 1 | Unchanged Low |
| Q9HB15 | 436.9131 | −1.43E−01 | −2.14E−01 | 1 | Unchanged Low |
| Q9Y3B4 | 350.4808 | 5.61E−01 | 1.87E−01 | 1 | Unchanged Low |
| Q14107 | 407.3091 | 1.52E−01 | 6.52E−02 | 1 | Unchanged Low |
| P52435 | 472.7272 | −2.94E−01 | −1.93E−01 | 1 | Unchanged Low |
| Q9Y262 | 444.385 | −4.58E−01 | −9.28E−01 | 1 | Unchanged Low |
| P10163 | 401.2036 | −2.74E−01 | −1.05E+00 | 1 | Unchanged Low |
| P38378 | 478.7225 | −5.57E−01 | −7.86E−01 | 1 | Unchanged Low |
| P48735 | 456.8577 | −4.74E−01 | −8.28E−01 | 1 | Unchanged Low |
| Q9NPF5 | 421.964 | −6.26E−02 | −2.41E−01 | 1 | Unchanged Low |
| O14616 | 324.9789 | 1.15E+00 | 8.38E−01 | 1 | Unchanged Low |
| Q00839 | 419.7665 | 5.44E−02 | −2.18E−02 | 1 | Unchanged Low |
| O95563 | 404.2972 | 1.47E−01 | −5.96E−03 | 1 | Unchanged Low |
| Q92974 | 233.2947 | 1.61E+00 | −1.95E−01 | 1 | Unchanged Low |
| Q9UEE5 | 434.4129 | 1.24E−01 | 2.46E−01 | 1 | Unchanged Low |
| Q9NX81 | 271.6819 | 1.31E+00 | 2.33E−01 | 1 | Unchanged Low |
| Q9NXF9 | 421.54 | −1.37E−01 | −4.38E−01 | 1 | Unchanged Low |
| P98171 | 345.0241 | 2.39E−01 | −6.77E−01 | 1 | Unchanged Low |
| P56192 | 331.1505 | 7.29E−01 | 1.63E−01 | 1 | Unchanged Low |
| O95411 | 326.6136 | 9.02E−01 | 4.10E−01 | 1 | Unchanged Low |
| P09132 | 419.7603 | 9.38E−01 | 1.38E+00 | 1 | Unchanged Low |
| Q92537 | 319.914 | 5.86E−01 | −3.30E−01 | 1 | Unchanged Low |
| P49761 | 486.0405 | −6.88E−02 | 2.91E−01 | 1 | Unchanged Low |
| Q9HDC4 | 391.3835 | 3.41E−01 | 1.62E−01 | 1 | Unchanged Low |
| P98160 | 439.0897 | −2.01E−01 | −4.44E−01 | 1 | Unchanged Low |
| Q9NQV6 | 441.3923 | 2.55E−01 | 4.79E−01 | 1 | Unchanged Low |
| Q9NRX6 | 370.1383 | 4.42E−01 | 8.82E−02 | 1 | Unchanged Low |
| O76084 | 374.6479 | −9.29E−02 | −1.16E+00 | 1 | Unchanged Low |
| P23197 | 428.3076 | −1.22E−01 | −4.09E−01 | 1 | Unchanged Low |
| Q99439 | 391.978 | 4.38E−02 | −4.96E−01 | 1 | Unchanged Low |
| O14656 | 463.6417 | 9.27E−02 | 3.59E−01 | 1 | Unchanged Low |
| Q9NPJ3 | 428.1718 | 1.09E−01 | 6.55E−02 | 1 | Unchanged Low |
| P41226 | 361.2685 | 5.70E−01 | 1.95E−01 | 1 | Unchanged Low |
| Q9Y478 | 304.7576 | 5.35E−01 | −8.93E−01 | 1 | Unchanged Low |
| | 348.2928 | 3.45E−01 | −4.73E−01 | 1 | Unchanged Low |
| Q12904 | 426.5346 | 2.50E−01 | 2.94E−01 | 1 | Unchanged Low |
| P20264 | 402.9521 | −3.44E−02 | −6.02E−01 | 1 | Unchanged Low |
| Q15428 | 355.7289 | 6.60E−01 | 2.36E−01 | 1 | Unchanged Low |
| P49771 | 439.4747 | 7.84E−03 | −1.10E−01 | 1 | Unchanged Low |
| P49005 | 349.3417 | 3.16E−01 | −6.49E−01 | 1 | Unchanged Low |
| Q99879 | 420.7323 | 4.74E−01 | 5.69E−01 | 1 | Unchanged Low |
| P49821 | 452.5721 | −1.70E−01 | −3.86E−01 | 1 | Unchanged Low |
| P10644 | 464.5909 | 1.12E−01 | 3.00E−01 | 1 | Unchanged Low |
| Q9Y5J4 | 301.448 | 1.64E+00 | 1.06E+00 | 1 | Unchanged Low |
| P31040 | 492.7974 | −5.57E−01 | −9.36E−01 | 1 | Unchanged Low |
| Q9UHA2 | 458.4094 | 5.73E−02 | 1.30E−01 | 1 | Unchanged Low |
| Q9UBF6 | 390.7196 | 5.34E−01 | 3.40E−01 | 1 | Unchanged Low |
| Q9Y5S2 | 430.2759 | 2.40E−01 | 2.08E−01 | 1 | Unchanged Low |
| P20809 | 332.3748 | 1.99E−01 | −1.51E+00 | 1 | Unchanged Low |
| Q13526 | 417.3666 | 4.59E−01 | 4.66E−01 | 1 | Unchanged Low |
| Q9NWQ9 | 488.6936 | 8.93E−02 | 4.22E−01 | 1 | Unchanged Low |
| Q9Y6J0 | 350.127 | −4.84E−02 | −2.02E+00 | 1 | Unchanged Low |
| Q9Y4Z6 | 283.3006 | 1.99E+00 | 1.26E+00 | 1 | Unchanged Low |
| Q01433 | 400.6864 | 3.82E−01 | 1.47E−01 | 1 | Unchanged Low |
| O43181 | 436.6589 | 1.41E−01 | 5.03E−02 | 1 | Unchanged Low |
| Q9NWT5 | 445.8286 | 3.77E−01 | 5.61E−01 | 1 | Unchanged Low |
| O75431 | 395.1258 | 7.05E−01 | 6.48E−01 | 1 | Unchanged Low |
| P09543 | 409.9513 | 8.39E−01 | 9.80E−01 | 1 | Unchanged Low |
| Q9BTE1 | 494.9234 | −2.83E−01 | −2.91E−01 | 1 | Unchanged Low |
| P16152 | 312.5841 | 9.85E−01 | 3.30E−02 | 1 | Unchanged Low |
| O15121 | 333.1906 | 8.11E−01 | 3.08E−02 | 1 | Unchanged Low |
| O60909 | 403.197 | 3.46E−01 | 4.98E−02 | 1 | Unchanged Low |
| O75223 | 411.9427 | −8.46E−02 | −8.39E−01 | 1 | Unchanged Low |
| Q9BQA1 | 382.8783 | 2.98E−01 | −3.22E−01 | 1 | Unchanged Low |
| O43427 | 371.8858 | 5.29E−01 | 6.63E−03 | 1 | Unchanged Low |
| Q9BWG6 | 457.7257 | 5.98E−01 | 9.45E−01 | 1 | Unchanged Low |
| Q9Y6R2 | 386.6911 | 4.77E−01 | 6.13E−02 | 1 | Unchanged Low |
| Q9HBK7 | 442.4205 | 3.59E−01 | 4.11E−01 | 1 | Unchanged Low |
| P52758 | 371.3427 | 5.36E−01 | −3.19E−02 | 1 | Unchanged Low |
| P47897 | 412.2837 | 2.40E−01 | −1.36E−01 | 1 | Unchanged Low |
| Q9BWQ6 | 415.0843 | 7.12E−01 | 7.43E−01 | 1 | Unchanged Low |
| P35325 | 483.0992 | 2.80E−01 | 5.82E−01 | 1 | Unchanged Low |
| Q9HA68 | 471.3877 | −2.29E−01 | −5.38E−01 | 1 | Unchanged Low |
| P49366 | 472.2313 | 5.17E−02 | 6.79E−02 | 1 | Unchanged Low |
| Q9UKZ7 | 477.7496 | −7.79E−02 | −1.67E−01 | 1 | Unchanged Low |
| P01730 | 475.788 | −1.09E−01 | −2.57E−01 | 1 | Unchanged Low |
| P20674 | 454.7764 | −9.34E−02 | −4.63E−01 | 1 | Unchanged Low |
| Q12828 | 418.2868 | 5.48E−02 | −5.69E−01 | 1 | Unchanged Low |
| Q9Y6A4 | 442.0882 | 4.18E−01 | 4.32E−01 | 1 | Unchanged Low |
| O60888 | 361.3342 | 6.88E−01 | 2.62E−02 | 1 | Unchanged Low |
| O75716 | 408.0382 | 4.02E−01 | 5.66E−02 | 1 | Unchanged Low |
| P30711 | 447.6268 | 5.56E−01 | 7.02E−01 | 1 | Unchanged Low |
| Q15008 | 488.0851 | 2.01E−02 | 9.00E−02 | 1 | Unchanged Low |
| P17174 | 373.5229 | 2.32E−01 | −8.84E−01 | 1 | Unchanged Low |
| P13798 | 400.3314 | 4.08E−01 | −5.51E−02 | 1 | Unchanged Low |
| Q9P2R8 | 488.4865 | 4.52E−02 | 1.23E−01 | 1 | Unchanged Low |
| Q9UNT1 | 366.9843 | 8.51E−01 | 3.68E−01 | 1 | Unchanged Low |
| O75607 | 369.1972 | 4.71E−01 | −3.66E−01 | 1 | Unchanged Low |
| O94925 | 456.0458 | 2.46E−01 | 1.98E−01 | 1 | Unchanged Low |
| O95390 | 359.3416 | 8.87E−01 | 2.92E−01 | 1 | Unchanged Low |
| O75956 | 498.9664 | −1.01E−01 | −1.22E−01 | 1 | Unchanged Low |
| O95573 | 339.2885 | 4.67E−01 | −1.02E+00 | 1 | Unchanged Low |
| Q9BXH1 | 472.8963 | −3.75E−03 | −1.88E−01 | 1 | Unchanged Low |
| P13662 | 454.7336 | 3.27E−01 | 2.87E−01 | 1 | Unchanged Low |
| Q92793 | 478.8243 | 7.73E−02 | 1.53E−02 | 1 | Unchanged Low |
| Q9P0H6 | 456.7944 | 2.85E−01 | 2.06E−01 | 1 | Unchanged Low |
| Q9P0R3 | 447.9021 | 5.47E−01 | 5.87E−01 | 1 | Unchanged Low |
| O75094 | 426.952 | 5.21E−01 | 3.42E−01 | 1 | Unchanged Low |
| Q9P011 | 498.5084 | 1.11E−01 | 2.24E−01 | 1 | Unchanged Low |
| Q92504 | 362.6669 | 9.83E−01 | 4.28E−01 | 1 | Unchanged Low |
| O95190 | 453.1247 | 2.41E−01 | 6.55E−02 | 1 | Unchanged Low |
| Q9Y230 | 379.9805 | 5.41E−01 | −1.89E−01 | 1 | Unchanged Low |
| Q9BSG0 | 426.9191 | 6.17E−01 | 4.98E−01 | 1 | Unchanged Low |
| Q9Y366 | 409.8539 | 8.41E−01 | 7.04E−01 | 1 | Unchanged Low |
| Q14094 | 420.31 | 2.94E−01 | −2.05E−01 | 1 | Unchanged Low |
| P43403 | 430.7684 | 3.38E−01 | −1.60E−03 | 1 | Unchanged Low |
| P52803 | 391.4643 | 4.87E−01 | −1.79E−01 | 1 | Unchanged Low |
| O95402 | 222.265 | 2.24E+00 | −8.52E−01 | 1 | Unchanged Low |
| O00233 | 494.287 | −2.12E−01 | −5.45E−01 | 1 | Unchanged Low |
| Q9H1K6 | 498.6205 | 2.16E−01 | 3.73E−01 | 1 | Unchanged Low |
| Q9BXL6 | 483.629 | 2.42E−01 | 2.99E−01 | 1 | Unchanged Low |
| Q9H173 | 469.6418 | 1.41E−01 | −2.46E−02 | 1 | Unchanged Low |
| Q9NV83 | 475.4325 | 2.52E−01 | 2.30E−01 | 1 | Unchanged Low |
| P11926 | 486.3355 | −2.27E−01 | −7.07E−01 | 1 | Unchanged Low |
| Q04743 | 449.3322 | −7.14E−02 | −7.73E−01 | 1 | Unchanged Low |
| Q9BVK2 | 425.9595 | 3.80E−01 | −2.18E−02 | 1 | Unchanged Low |
| O43251 | 461.1452 | 5.49E−01 | 6.12E−01 | 1 | Unchanged Low |
| Q99829 | 377.1688 | 5.17E−01 | −4.09E−01 | 1 | Unchanged Low |
| O95777 | 403.8183 | 3.77E−01 | −3.36E−01 | 1 | Unchanged Low |
| Q9UN53 | 414.6963 | 6.36E−01 | 3.13E−01 | 1 | Unchanged Low |
| P24310 | 327.1972 | 1.47E+00 | 6.46E−01 | 1 | Unchanged Low |
| Q9H929 | 465.7217 | 3.97E−01 | 3.67E−01 | 1 | Unchanged Low |
| P10768 | 402.8142 | −2.24E−01 | −2.55E+00 | 1 | Unchanged Low |
| O95287 | 472.3714 | 5.61E−01 | 6.95E−01 | 1 | Unchanged Low |
| Q01105 | 414.2142 | 6.37E−01 | 2.94E−01 | 1 | Unchanged Low |
| Q9UK45 | 483.7088 | 3.98E−01 | 5.04E−01 | 1 | Unchanged Low |
| P35611 | 488.7819 | 2.37E−01 | 2.54E−01 | 1 | Unchanged Low |
| O14681 | 430.8211 | 1.94E−01 | −4.77E−01 | 1 | Unchanged Low |
| P80303 | 349.5918 | 8.03E−01 | −3.28E−01 | 1 | Unchanged Low |
| Q9NWC0 | 476.3476 | 2.53E−01 | 1.34E−01 | 1 | Unchanged Low |
| P00367 | 394.0342 | 5.23E−01 | −2.50E−01 | 1 | Unchanged Low |
| O00469 | 497.4006 | −5.05E−01 | −1.63+00 | 1 | Unchanged Low |
| Q9UI04 | 406.7523 | 5.15E−01 | −1.17E−01 | 1 | Unchanged Low |
| P20338 | 488.5317 | 9.39E−02 | −9.49E−02 | 1 | Unchanged Low |
| Q9HCU8 | 489.0161 | 2.51E−02 | −2.53E−01 | 1 | Unchanged Low |
| P00519 | 352.0574 | 1.17E+00 | 3.73E−01 | 1 | Unchanged Low |
| Q13880 | 453.0588 | −1.28E−01 | −1.12E+00 | 1 | Unchanged Low |
| P25789 | 498.9408 | 2.70E−01 | 2.90E−01 | 1 | Unchanged Low |
| O43805 | 477.7167 | 5.17E−01 | 5.50E−01 | 1 | Unchanged Low |
| O75817 | 445.0255 | 4.89E−01 | 2.08E−01 | 1 | Unchanged Low |
| Q9UBI1 | 430.701 | 6.45E−01 | 3.45E−01 | 1 | Unchanged Low |
| P03950 | 374.4251 | 4.18E−01 | −9.79E−01 | 1 | Unchanged Low |
| P78423 | 493.2272 | 1.39E+00 | 1.89E+00 | 1 | Unchanged Low |
| Q9P1D0 | 220.9467 | 3.20E+00 | 3.36E−01 | 1 | Unchanged Low |
| P03999 | 229.0786 | 2.19E+00 | −1.62E+00 | 1 | Unchanged Low |
| O95793 | 483.6536 | 3.56E−01 | 2.54E−01 | 1 | Unchanged Low |
| P22750 | 393.9782 | 8.57E−01 | 2.19E−01 | 1 | Unchanged Low |
| | 481.1517 | 3.27E−01 | 1.29E−01 | 1 | Unchanged Low |
| Q9NRX8 | 411.9671 | 1.69E+00 | 1.63E+00 | 1 | Unchanged Low |
| Q9UHQ3 | 410.1503 | 2.17E−01 | −1.13E+00 | 1 | Unchanged Low |
| Q96S52 | 412.8095 | 4.34E−01 | −4.99E−01 | 1 | Unchanged Low |
| Q9H3F6 | 462.5636 | 3.10E−01 | −1.63E−01 | 1 | Unchanged Low |
| P37198 | 492.7531 | 3.63E−01 | 2.21E−01 | 1 | Unchanged Low |
| Q9H1D4 | 486.2744 | 3.59E−01 | 1.47E−01 | 1 | Unchanged Low |
| O75350 | 489.9278 | 2.02E−01 | −1.40E−01 | 1 | Unchanged Low |
| Q9P024 | 467.714 | 5.83E−01 | 3.78E−01 | 1 | Unchanged Low |
| Q9Y224 | 492.0453 | 3.05E−01 | 7.30E−02 | 1 | Unchanged Low |
| Q13361 | 368.553 | 1.52E+00 | 8.67E−01 | 1 | Unchanged Low |
| Q02535 | 272.6788 | 2.73E+00 | 1.05E+00 | 1 | Unchanged Low |
| O95864 | 323.8122 | 9.07E−01 | −1.40E+00 | 1 | Unchanged Low |
| P53621 | 480.1571 | 4.54E−01 | 2.01E−01 | 1 | Unchanged Low |
| P35638 | 401.9484 | 8.09E−01 | −2.12E−02 | 1 | Unchanged Low |
| P22307 | 426.421 | 6.84E−01 | 5.58E−03 | 1 | Unchanged Low |
| Q92543 | 417.8592 | 7.95E−01 | 1.15E−01 | 1 | Unchanged Low |
| P78330 | 347.8406 | 1.17E+00 | −2.03E−01 | 1 | Unchanged Low |
| Q9UNX3 | 472.8205 | 5.12E−01 | 1.48E−01 | 1 | Unchanged Low |
| P49189 | 434.2707 | 3.76E−01 | −6.87E−01 | 1 | Unchanged Low |
| Q9ULX0 | 489.9828 | 2.47E−01 | −2.81E−01 | 1 | Unchanged Low |
| O15353 | 495.2368 | 5.09E−01 | 2.85E−01 | 1 | Unchanged Low |
| Q9NRP2 | 461.731 | 6.03E−01 | 1.36E−01 | 1 | Unchanged Low |
| Q16280 | 469.6748 | 3.34E−01 | −3.66E−01 | 1 | Unchanged Low |
| P04183 | 407.8979 | 7.63E−01 | −2.64E−01 | 1 | Unchanged Low |
| Q12824 | 454.3734 | 1.43E+00 | 1.34E+00 | 1 | Unchanged Low |
| Q99719 | 474.0479 | 4.97E−01 | −3.19E−02 | 1 | Unchanged Low |
| P46100 | 356.9983 | 9.40E−01 | −8.93E−01 | 1 | Unchanged Low |
| Q16610 | 389.6774 | 1.32E+00 | 4.89E−01 | 1 | Unchanged Low |
| P39656 | 466.4028 | 4.64E−01 | −2.39E−01 | 1 | Unchanged Low |
| P53814 | 461.5158 | 2.09E−01 | −9.44E−01 | 1 | Unchanged Low |
| Q9P0N4 | 499.258 | −1.90E+02 | −1.09E−00 | 1 | Unchanged Low |
| Q9BXZ1 | 477.1895 | 3.76E−01 | −3.62E−01 | 1 | Unchanged Low |
| Q9Y2Q4 | 444.641 | 8.42E−01 | 2.14E−01 | 1 | Unchanged Low |
| O15527 | 406.0683 | 8.73E−01 | −2.27E−1 | 1 | Unchanged Low |
| Q04323 | 454.2076 | 2.95E−01 | −9.61E−01 | 1 | Unchanged Low |
| P12004 | 416.0883 | 8.84E−01 | −1.31E−01 | 1 | Unchanged Low |
| Q99720 | 452.6168 | 7.57E−01 | 5.85E−02 | 1 | Unchanged Low |
| P43307 | 389.0352 | 1.45E+00 | 5.10E−01 | 1 | Unchanged Low |
| Q9H3Y8 | 362.4108 | 1.49E+00 | 1.55E−01 | 1 | Unchanged Low |
| Q9NRX2 | 405.9846 | 1.14E+00 | 1.01E−01 | 1 | Unchanged Low |
| Q9P2X0 | 451.8107 | 1.16E+00 | 5.95E−01 | 1 | Unchanged Low |
| Q9Y5Z8 | 465.0297 | 8.23E−01 | 1.22E−01 | 1 | Unchanged Low |
| P78524 | 425.1425 | 9.06E−01 | −2.50E−01 | 1 | Unchanged Low |
| P55145 | 496.4892 | 6.68E−01 | 2.26E−01 | 1 | Unchanged Low |
| P33551 | 440.861 | 1.18E+00 | 2.59E−01 | 1 | Unchanged Low |
| Q9BVQ0 | 259.6855 | 3.98E+00 | 3.32E−01 | 1 | Unchanged Low |
| P34897 | 472.1838 | 6.37E−01 | −6.05E−01 | 1 | Unchanged Low |
| Q9Y5B4 | 491.1814 | 1.35E+00 | 9.52E−01 | 1 | Unchanged Low |
| Q9Y2V5 | 429.636 | 1.52E+00 | 3.94E−01 | 1 | Unchanged Low |
| Q13953 | 499.9658 | 1.09E+00 | 3.21E−01 | 1 | Unchanged Low |
| Q92686 | 455.4495 | 1.15E+00 | −1.45E−01 | 1 | Unchanged Low |
| O60565 | 346.3594 | 2.87E+00 | 5.05E−01 | 1 | Unchanged Low |
| Q9Y4M4 | 481.2315 | 1.29E+00 | −4.63E−01 | 1 | Unchanged Low |
| P16581 | 454.0605 | 3.00E+00 | 1.79E+01 | 1 | Unchanged Low |
| O14733 | 375.5944 | 3.17E+00 | 5.02E−01 | 1 | Unchanged Low |
| Q16623 | 498.3425 | 1.20E+00 | −1.29E+00 | 1 | Unchanged Low |
| Q9P1M5 | 450.1426 | 5.05E+00 | −1.01E+00 | 2 | Unchanged Low |
| O60613 | 1205.045 | 7.39E−02 | −1.38E−01 | 2 | Unchanged Medium |
| Q16698 | 597.2594 | 3.89E−01 | 5.88E−01 | 2 | Unchanged Medium |
| Q9HBA8 | 557.2677 | −2.95E−01 | −1.47E+00 | 2 | Unchanged Medium |
| Q9P0I2 | 617.4113 | 1.70E−01 | 3.39E−01 | 2 | Unchanged Medium |
| Q9UL53 | 560.6322 | −5.91E−01 | −1.03E−01 | 2 | Unchanged Medium |
| P49902 | 900.9404 | −1.24E+00 | 2.32E−01 | 2 | Unchanged Medium |
| O95336 | 697.4428 | 6.32E−01 | 5.27E−01 | 2 | Unchanged Medium |
| Q03393 | 525.0184 | 5.31E−01 | 9.66E−01 | 2 | Unchanged Medium |
| Q13443 | 744.5037 | −1.55E+00 | −1.44E+00 | 2 | Unchanged Medium |
| O75173 | 1033.446 | 6.74E−01 | 4.29E−01 | 2 | Unchanged Medium |
| P51572 | 843.6943 | −5.85E−01 | −1.48E−01 | 2 | Unchanged Medium |
| P42765 | 1111.329 | 5.03E−01 | 5.92E−01 | 2 | Unchanged Medium |
| P24666 | 1833.204 | 2.03E−01 | 2.59E−01 | 2 | Unchanged Medium |
| Q92688 | 531.9498 | 8.28E−01 | −7.04E−01 | 2 | Unchanged Medium |
| O14639 | 701.2899 | −1.91E+00 | −1.78E+00 | 2 | Unchanged Medium |
| O15143 | 519.3549 | −9.96E−01 | −4.40E−01 | 2 | Unchanged Medium |
| O15509 | 868.6913 | −1.71E−01 | −3.18E−02 | 2 | Unchanged Medium |
| O15511 | 605.8842 | −6.51E−01 | −2.42E−01 | 2 | Unchanged Medium |
| P12718 | 1175.856 | 1.84E+00 | 5.32E−01 | 2 | Unchanged Medium |
| Q9P0I6 | 1014.448 | 4.11E−01 | −1.79E−01 | 2 | Unchanged Medium |
| O43747 | 685.2957 | −1.40E+00 | −1.05E+00 | 2 | Unchanged Medium |
| P20172 | 1617.354 | 8.28E−01 | −7.63E−03 | 2 | Unchanged Medium |
| O14617 | 510.4994 | −9.28E−01 | −4.57E−01 | 2 | Unchanged Medium |
| P07741 | 1190.468 | −1.19E+01 | −1.13E+00 | 2 | Unchanged Medium |
| P00568 | 1052.361 | −2.76E+00 | −2.59E+00 | 2 | Unchanged Medium |
| Q9U1J7 | 828.3999 | −1.95E−01 | 4.87E−01 | 2 | Unchanged Medium |
| P30566 | 545.4304 | 6.41E−01 | 3.27E−01 | 2 | Unchanged Medium |
| Q16186 | 602.2578 | −1.15E−01 | −2.95E−02 | 2 | Unchanged Medium |
| Q9NVF6 | 582.9297 | 5.90E−01 | 4.72E−01 | 2 | Unchanged Medium |
| P26438 | 809.1342 | 2.60E−01 | 1.61E−01 | 2 | Unchanged Medium |
| P40616 | 1064.016 | 4.65E−01 | 5.64E−01 | 2 | Unchanged Medium |
| P36404 | 1723.74 | 9.61E−02 | −1.77E−01 | 2 | Unchanged Medium |
| P56559 | 550.5764 | 2.28E−01 | 3.00E−01 | 2 | Unchanged Medium |
| P35368 | 1426.069 | −1.02E−01 | 7.34E−02 | 2 | Unchanged Medium |
| P15144 | 563.2289 | −1.04E+00 | −2.15E+00 | 2 | Unchanged Medium |
| P30038 | 811.7082 | −8.44E−01 | −9.81E−01 | 2 | Unchanged Medium |
| P14550 | 683.9189 | 1.35E−01 | 6.74E−01 | 2 | Unchanged Medium |
| O43488 | 606.6855 | 4.54E−01 | −5.74E−02 | 2 | Unchanged Medium |
| P09972 | 960.156 | 1.09E−01 | 2.63E−01 | 2 | Unchanged Medium |
| O43590 | 783.883 | −6.13E−01 | 4.68E−01 | 2 | Unchanged Medium |
| Q08117 | 1476.318 | −5.45E−01 | −3.16E−01 | 2 | Unchanged Medium |
| Q13438 | 699.6136 | 2.86E−01 | −4.36E−02 | 2 | Unchanged Medium |
| O96018 | 876.6611 | 5.52E−01 | −2.38E−02 | 2 | Unchanged Medium |
| Q13564 | 679.5497 | 4.21E−01 | 1.78E−01 | 2 | Unchanged Medium |
| Q9Y679 | 1160.442 | −2.21E−01 | 8.81E−02 | 2 | Unchanged Medium |
| Q9UKB4 | 622.7291 | 1.19E−01 | 5.66E−01 | 2 | Unchanged Medium |
| Q13725 | 1304.886 | −4.98E−01 | −1.36E−01 | 2 | Unchanged Medium |
| P08133 | 515.5041 | 1.80E+00 | 7.08E−01 | 2 | Unchanged Medium |
| P20073 | 517.0979 | −6.29E−01 | −4.36E−01 | 2 | Unchanged Medium |
| P03971 | 909.9247 | −8.32E−01 | −2.67E−01 | 2 | Unchanged Medium |
| P30041 | 1824.225 | 1.30E+00 | −1.12E−01 | 2 | Unchanged Medium |
| P27695 | 718.2149 | 3.27E−01 | −2.85E−02 | 2 | Unchanged Medium |
| Q9Y4J7 | 688.7802 | −3.65E+00 | −4.76E+00 | 2 | Unchanged Medium |
| Q9Y5L7 | 890.7729 | 1.11E+00 | −1.86E−01 | 2 | Unchanged Medium |
| O94778 | 1455.01 | −7.13E−04 | −5.92E−02 | 2 | Unchanged Medium |
| O15296 | 718.1334 | −1.99E−01 | −4.45E−01 | 2 | Unchanged Medium |
| P48444 | 1288.649 | 8.92E−02 | −1.07E−01 | 2 | Unchanged Medium |
| P00966 | 1199.548 | −1.45E−01 | 6.08E−01 | 2 | Unchanged Medium |
| P42024 | 1518.804 | 7.54E−01 | 7.10E−01 | 2 | Unchanged Medium |
| O43681 | 620.0089 | 1.40E−01 | 4.23E−01 | 2 | Unchanged Medium |
| Q9ULX3 | 611.0757 | −5.17E−01 | −2.92E−01 | 2 | Unchanged Medium |
| O96030 | 749.363 | −4.66E−01 | −2.76E−01 | 2 | Unchanged Medium |
| P15289 | 1294.172 | −6.96E−01 | −4.14E−01 | 2 | Unchanged Medium |
| P08243 | 1024.719 | 7.89E−01 | −4.72E−01 | 2 | Unchanged Medium |
| O95630 | 753.7548 | 1.43E−01 | 4.01E−01 | 2 | Unchanged Medium |
| O95135 | 530.0852 | −1.40E+00 | −1.39E+00 | 2 | Unchanged Medium |
| O14530 | 993.482 | −1.11E−01 | 7.45E−01 | 2 | Unchanged Medium |
| P30049 | 511.6658 | −5.02E−01 | −4.16E−01 | 2 | Unchanged Medium |
| Q9UII2 | 878.5341 | −2.76E−01 | 6.49E−01 | 2 | Unchanged Medium |
| Q9Y5K8 | 1232.91 | 4.53E−02 | 7.43E−01 | 2 | Unchanged Medium |
| O75787 | 947.5842 | 4.07E−01 | 2.13E−01 | 2 | Unchanged Medium |
| P05023 | 841.8422 | −8.74E−01 | −1.33E+00 | 2 | Unchanged Medium |
| P45844 | 616.8957 | −5.11E−01 | −1.18E+00 | 2 | Unchanged Medium |
| Q9HAP7 | 1730.54 | 3.68E−01 | 2.07E−01 | 2 | Unchanged Medium |
| O94812 | 961.2437 | −3.77E−01 | −1.27E−01 | 2 | Unchanged Medium |
| P35613 | 647.414 | 5.20E−01 | −3.50E−01 | 2 | Unchanged Medium |
| Q9BSR6 | 1015.173 | 1.64E−01 | 6.88E−01 | 2 | Unchanged Medium |
| O00512 | 504.741 | −4.08E+00 | −4.15E+00 | 2 | Unchanged Medium |
| Q12983 | 767.5569 | 3.77E−01 | −4.80E−01 | 2 | Unchanged Medium |
| Q92934 | 623.2249 | 1.64E−01 | 4.33E−01 | 2 | Unchanged Medium |
| Q07817 | 1890.905 | −9.10E−01 | 2.85E−01 | 2 | Unchanged Medium |
| O43892 | 539.1375 | −2.55E−01 | −6.63E−01 | 2 | Unchanged Medium |
| P53004 | 523.696 | 6.39E−02 | 2.15E−01 | 2 | Unchanged Medium |
| O60629 | 956.1289 | 8.33E−01 | 9.53E−01 | 2 | Unchanged Medium |
| Q9HBX3 | 590.2997 | −5.25E+00 | −4.95E+00 | 2 | Unchanged Medium |
| P56945 | 1312.13 | −3.34E−02 | −4.14E−02 | 2 | Unchanged Medium |
| O15255 | 837.48 | 7.12E−01 | −3.48E−01 | 2 | Unchanged Medium |
| P12830 | 835.8959 | −1.16E+00 | −2.45E−01 | 2 | Unchanged Medium |
| P19022 | 541.7151 | 1.15E+00 | 6.79E−01 | 2 | Unchanged Medium |
| P22223 | 823.6929 | −1.27E+00 | 4.22E−01 | 2 | Unchanged Medium |
| P22676 | 1797.589 | 2.49E−01 | −2.52E−01 | 2 | Unchanged Medium |
| Q99828 | 1096.979 | −4.31E−01 | 2.26E−01 | 2 | Unchanged Medium |
| P02593 | 949.1389 | −1.30E−01 | −4.51E−01 | 2 | Unchanged Medium |
| Q13942 | 746.0174 | −5.13E−02 | −9.59E−01 | 2 | Unchanged Medium |
| P27824 | 1470.4 | 4.85E−02 | −1.19E−01 | 2 | Unchanged Medium |
| P17655 | 685.847 | −2.90E−01 | 3.17E−01 | 2 | Unchanged Medium |
| P04632 | 1490.643 | −3.16E−01 | 7.36E−02 | 2 | Unchanged Medium |
| Q15417 | 750.6841 | −3.06E−01 | −5.95E−01 | 2 | Unchanged Medium |
| Q9UEG5 | 518.1163 | −5.17E−01 | −3.73E−01 | 2 | Unchanged Medium |
| P52907 | 705.5063 | −5.36E−01 | −3.11E−01 | 2 | Unchanged Medium |
| Q9GZX3 | 824.5253 | −7.81E−02 | 2.28E−01 | 2 | Unchanged Medium |
| Q16619 | 1420.784 | 7.72E−02 | −2.72E−01 | 2 | Unchanged Medium |
| Q9UBD9 | 581.3157 | 4.38E−01 | 1.30E+00 | 2 | Unchanged Medium |
| Q15699 | 817.569 | 5.71E−01 | −3.26E−01 | 2 | Unchanged Medium |
| P48729 | 914.9218 | 3.86E−01 | 7.36E−01 | 2 | Unchanged Medium |
| P48730 | 1073.796 | 2.24E−01 | 2.84E−01 | 2 | Unchanged Medium |
| P19138 | 513.9702 | −6.66E−02 | 3.03E−01 | 2 | Unchanged Medium |
| P49662 | 1831.485 | −9.85E−01 | 2.90E−01 | 2 | Unchanged Medium |
| P21964 | 1046.831 | 8.63E−01 | 7.51E−01 | 2 | Unchanged Medium |
| P26232 | 774.9896 | 2.22E−02 | −1.54E−01 | 2 | Unchanged Medium |
| P35222 | 1121.277 | 1.87E−01 | 4.14E−01 | 2 | Unchanged Medium |
| P07339 | 1111.314 | −1.44E−01 | −4.04E−01 | 2 | Unchanged Medium |
| Q99967 | 1711.366 | 7.95E−01 | 3.02E−01 | 2 | Unchanged Medium |
| P49715 | 937.3612 | 2.44E−01 | 1.26E−03 | 2 | Unchanged Medium |
| P53567 | 1191.643 | 9.38E−01 | 4.92E−01 | 2 | Unchanged Medium |
| O95627 | 716.7046 | 1.91E−01 | 4.89E−01 | 2 | Unchanged Medium |
| Q04900 | 664.5986 | −1.55E−01 | −3.97E−01 | 2 | Unchanged Medium |
| P07766 | 1953.844 | 5.48E−01 | −3.59E−01 | 2 | Unchanged Medium |
| P34810 | 1617.586 | −9.27E−01 | −1.16E+00 | 2 | Unchanged Medium |
| P18582 | 1279.761 | 2.95E−01 | −4.75E−01 | 2 | Unchanged Medium |
| Q16181 | 540.3444 | 8.16E−01 | 5.42E−02 | 2 | Unchanged Medium |
| Q16543 | 1158.574 | 2.99E−01 | −4.35E−02 | 2 | Unchanged Medium |
| O14735 | 579.4931 | 3.67E−01 | 2.29E+00 | 2 | Unchanged Medium |
| P30260 | 1334.733 | 4.70E−01 | 4.74E−01 | 2 | Unchanged Medium |
| P49427 | 942.0784 | 7.74E−03 | 7.66E−01 | 2 | Unchanged Medium |
| P29373 | 1126.373 | 1.39E−01 | −6.11E−01 | 2 | Unchanged Medium |
| P41208 | 629.3345 | 3.28E−01 | 3.18E−01 | 2 | Unchanged Medium |
| O00522 | 741.4854 | −2.67E−01 | −4.64E−01 | 2 | Unchanged Medium |
| O15183 | 632.0808 | 1.13E+00 | 5.98E−01 | 2 | Unchanged Medium |
| Q9Y2Z5 | 857.6492 | 7.06E−01 | −9.94E−02 | 2 | Unchanged Medium |
| Q9Y3C3 | 1572.302 | 7.89E−01 | 1.49E−01 | 2 | Unchanged Medium |
| Q9Y3D0 | 1992.502 | 3.37E−01 | 5.75E−01 | 2 | Unchanged Medium |
| Q9Y3E5 | 629.3057 | 9.09E−02 | 5.66E−01 | 2 | Unchanged Medium |
| Q9Y3E8 | 829.1206 | −1.33E−02 | 2.35E−01 | 2 | Unchanged Medium |
| Q9Y320 | 793.4541 | −8.22E−02 | 3.22E−01 | 2 | Unchanged Medium |
| Q9Y357 | 668.441 | −9.66E−01 | −8.87E−02 | 2 | Unchanged Medium |
| Q9Y360 | 832.2061 | 1.93E−02 | 2.17E−01 | 2 | Unchanged Medium |
| Q9Y512 | 957.4201 | 1.12E−01 | −1.71E−01 | 2 | Unchanged Medium |
| Q9Y390 | 1111.152 | −2.88E−01 | −7.96E−01 | 2 | Unchanged Medium |
| P40227 | 679.9043 | −7.72E−01 | −1.22E+00 | 2 | Unchanged Medium |
| Q99832 | 1274.983 | 3.79E−01 | −1.33E−01 | 2 | Unchanged Medium |
| Q9UBR5 | 1303.992 | 1.62E−02 | 7.85E−01 | 2 | Unchanged Medium |
| Q9UHN8 | 1611.343 | −3.42E−01 | 3.70E−01 | 2 | Unchanged Medium |
| P54105 | 532.9217 | 4.44E−01 | 4.80E−02 | 2 | Unchanged Medium |
| Q14781 | 753.0305 | −1.57E−01 | −8.68E−02 | 2 | Unchanged Medium |
| Q9BXS4 | 1050.603 | 4.91E−02 | 6.52E−01 | 2 | Unchanged Medium |
| Q9NPA0 | 897.3389 | −2.18E−01 | −2.60E−01 | 2 | Unchanged Medium |
| Q9UKR5 | 1207.747 | 1.21E−01 | −3.18E−02 | 2 | Unchanged Medium |
| P56378 | 694.9486 | 8.23E−01 | 4.55E−01 | 2 | Unchanged Medium |
| O95433 | 552.8428 | −5.80E−01 | −2.20E−01 | 2 | Unchanged Medium |
| Q9NV31 | 848.9729 | 1.24E−02 | −3.76E−02 | 2 | Unchanged Medium |
| Q9NVE2 | 596.0999 | −4.69E−01 | 4.96E−02 | 2 | Unchanged Medium |
| Q9UI05 | 1952.881 | −1.07E+00 | −2.36E−01 | 2 | Unchanged Medium |
| Q9P0A7 | 954.524 | 1.04E+00 | 8.93E−01 | 2 | Unchanged Medium |
| Q9Y3B1 | 1235.108 | −1.11E+00 | −4.60E−01 | 2 | Unchanged Medium |
| Q9BQ89 | 1211.641 | −2.58E−01 | −6.17E−01 | 2 | Unchanged Medium |
| Q96C58 | 940.0036 | −1.38E+00 | −2.10E−01 | 2 | Unchanged Medium |
| Q9Y5Z4 | 758.6366 | −1.30E+00 | 1.10E−01 | 2 | Unchanged Medium |
| Q00610 | 1427.503 | −1.90E−01 | −4.44E−01 | 2 | Unchanged Medium |
| P56749 | 1992.755 | −8.75E−01 | 1.34E−01 | 2 | Unchanged Medium |
| O43809 | 608.9825 | 9.40E−02 | −3.47E−01 | 2 | Unchanged Medium |
| O96005 | 1304.597 | 4.36E−02 | −2.15E−01 | 2 | Unchanged Medium |
| Q16740 | 543.0367 | −3.98E−01 | −2.45E−01 | 2 | Unchanged Medium |
| P55085 | 734 | −1.08E+00 | 2.39E−01 | 2 | Unchanged Medium |
| P13726 | 800.9723 | −1.29E+00 | −2.94E−01 | 2 | Unchanged Medium |
| Q9Y678 | 854.5161 | 3.08E−01 | −1.45E−01 | 2 | Unchanged Medium |
| Q15363 | 793.6589 | 3.01E−01 | 1.88E−01 | 2 | Unchanged Medium |
| O43513 | 623.9284 | 4.33E−01 | −2.99E−01 | 2 | Unchanged Medium |
| Q9Y281 | 678.6369 | 7.03E−01 | −1.31E+00 | 2 | Unchanged Medium |
| P04141 | 522.783 | 5.12E−01 | 3.47E+00 | 2 | Unchanged Medium |
| P02745 | 1042.465 | 2.24E−01 | 1.27E+00 | 2 | Unchanged Medium |
| P29279 | 734.8489 | 8.45E−01 | −1.52E−01 | 2 | Unchanged Medium |
| O43191 | 1062.925 | 6.06E−01 | 2.68E−01 | 2 | Unchanged Medium |
| Q99627 | 984.0634 | 6.01E−01 | 2.97E−01 | 2 | Unchanged Medium |
| O15387 | 533.8251 | 8.88E−02 | −6.30E−01 | 2 | Unchanged Medium |
| Q9ULV4 | 1385.839 | −2.26E−01 | −4.09E−01 | 2 | Unchanged Medium |
| Q9BR76 | 827.7864 | −3.65E−02 | 6.13E−01 | 2 | Unchanged Medium |
| Q14061 | 1026.374 | −2.54E−01 | 7.00E−01 | 2 | Unchanged Medium |
| Q9Y6B2 | 1323.308 | 1.07E+00 | 1.27E−01 | 2 | Unchanged Medium |
| Q9NY68 | 991.4995 | −8.09E−01 | −7.81E−01 | 2 | Unchanged Medium |
| P24385 | 1486.769 | −1.42E−01 | 8.21E−01 | 2 | Unchanged Medium |
| Q9NXT4 | 1620.99 | 2.03E−02 | 1.40E−01 | 2 | Unchanged Medium |
| P01034 | 1134.612 | 3.57E−01 | −1.37E−01 | 2 | Unchanged Medium |
| Q15828 | 1404.267 | −1.10E+00 | 1.53E+00 | 2 | Unchanged Medium |
| P01036 | 932.4892 | −2.38E−01 | −1.44E+00 | 2 | Unchanged Medium |
| P52943 | 883.82 | 5.48E−01 | −8.08E−01 | 2 | Unchanged Medium |
| P13498 | 707.7483 | −9.81E−01 | 5.73E−01 | 2 | Unchanged Medium |
| P00167 | 1565.893 | −1.08E+00 | −5.67E−01 | 2 | Unchanged Medium |
| P00001 | 502.4786 | −8.14E−02 | −3.09E−01 | 2 | Unchanged Medium |
| P10606 | 1851.225 | 6.14E−01 | 6.24E−01 | 2 | Unchanged Medium |
| P08574 | 517.4383 | −5.97E−01 | −5.93E−01 | 2 | Unchanged Medium |
| Q99426 | 673.9633 | 4.71E−01 | 2.85E−01 | 2 | Unchanged Medium |
| Q07065 | 987.5887 | 1.57E+00 | −6.78E−02 | 2 | Unchanged Medium |
| Q15038 | 754.4305 | −5.17E−01 | 1.40E−01 | 2 | Unchanged Medium |
| Q9H2L4 | 630.0414 | 9.35E−01 | 7.82E−01 | 2 | Unchanged Medium |
| Q9NPA8 | 1342.189 | 6.81E−01 | 7.32E−01 | 2 | Unchanged Medium |
| O60231 | 808.7915 | 8.50E−02 | 2.11E−01 | 2 | Unchanged Medium |
| Q9GZR7 | 854.0965 | −5.31E−02 | 2.93E−01 | 2 | Unchanged Medium |
| P17844 | 1530.749 | 1.72E−01 | −4.26E−02 | 2 | Unchanged Medium |
| Q08211 | 824.2193 | −2.41E−04 | −4.65E−01 | 2 | Unchanged Medium |
| Q9UJV9 | 1024.437 | −1.66E−01 | 3.28E−01 | 2 | Unchanged Medium |
| P51398 | 905.1904 | −1.76E−01 | 9.20E−02 | 2 | Unchanged Medium |
| Q13437 | 1596.672 | 1.37E−01 | −5.88E−02 | 2 | Unchanged Medium |
| O60735 | 609.2393 | −8.78E−01 | −7.75E−01 | 2 | Unchanged Medium |
| Q16854 | 910.8082 | 2.05E−01 | 4.43E−02 | 2 | Unchanged Medium |
| Q9Y295 | 676.9267 | −1.56E−01 | −2.17E−01 | 2 | Unchanged Medium |
| P00387 | 1290.593 | 3.42E−02 | −4.40E−01 | 2 | Unchanged Medium |
| Q9UHY9 | 741.1 | 7.87E−01 | 4.44E−01 | 2 | Unchanged Medium |
| P36957 | 577.6196 | −6.69E−01 | 1.88E−01 | 2 | Unchanged Medium |
| Q99075 | 518.9525 | −1.87E+00 | −1.78E−01 | 2 | Unchanged Medium |
| Q9UFN1 | 1127.658 | 6.04E−01 | −2.91E−02 | 2 | Unchanged Medium |
| O95882 | 870.3185 | −2.26E−01 | −1.22E−01 | 2 | Unchanged Medium |
| Q9Y269 | 998.4411 | 8.38E−01 | 7.17E−01 | 2 | Unchanged Medium |
| Q9NWE3 | 605.084 | 5.77E−01 | 8.76E−01 | 2 | Unchanged Medium |
| Q9H6Y7 | 1352.292 | −2.23E−01 | 8.55E−02 | 2 | Unchanged Medium |
| Q9Y3X0 | 588.5767 | 1.93E−01 | −2.21E−01 | 2 | Unchanged Medium |
| O60762 | 531.3373 | 5.74E−01 | 5.14E−01 | 2 | Unchanged Medium |
| P53805 | 515.0794 | 4.68E−01 | 8.42E−01 | 2 | Unchanged Medium |
| Q13597 | 636.966 | 6.87E−02 | 2.20E−01 | 2 | Unchanged Medium |
| Q9C005 | 1064.132 | 5.35E−01 | 5.76E−01 | 2 | Unchanged Medium |
| Q13448 | 1586.247 | 9.42E−01 | −1.27E−01 | 2 | Unchanged Medium |
| Q9UHF9 | 703.5318 | −1.86E+00 | −8.23E−01 | 2 | Unchanged Medium |
| P51452 | 576.1845 | −4.56E−01 | −1.02E−01 | 2 | Unchanged Medium |
| Q14203 | 553.5576 | 2.97E−01 | 6.06E−02 | 2 | Unchanged Medium |
| Q13561 | 1372.261 | −4.16E−01 | −1.84E−01 | 2 | Unchanged Medium |
| Q13409 | 620.6506 | −7.71E−01 | −3.94E−01 | 2 | Unchanged Medium |
| Q09472 | 1651.644 | −1.56E−01 | 5.00E−01 | 2 | Unchanged Medium |
| | 588.0816 | −4.51E−01 | 2.28E−02 | 2 | Unchanged Medium |
| P78365 | 907.5945 | −2.32E−01 | 2.54E−01 | 2 | Unchanged Medium |
| Q99848 | 802.3778 | −2.56E−01 | −6.79E−01 | 2 | Unchanged Medium |
| Q12805 | 597.7763 | −1.13E+00 | −1.60E+00 | 2 | Unchanged Medium |
| Q9Y6I3 | 621.9752 | −2.28E−01 | −5.60E−01 | 2 | Unchanged Medium |
| Q15717 | 831.3485 | −1.26E−02 | −8.94E−02 | 2 | Unchanged Medium |
| P13804 | 664.3533 | −7.96E−01 | −7.54E−01 | 2 | Unchanged Medium |
| Q14247 | 1063.9 | −1.80E−01 | −1.06E−01 | 2 | Unchanged Medium |
| P42892 | 737.1982 | 4.27E−01 | 4.08E−01 | 2 | Unchanged Medium |
| P30084 | 917.9548 | −1.14E−01 | 5.93E−01 | 2 | Unchanged Medium |
| P07099 | 1424.661 | −3.96E−02 | 5.83E−01 | 2 | Unchanged Medium |
| P29692 | 1061.631 | −2.59E−01 | −8.37E−01 | 2 | Unchanged Medium |
| P20042 | 1016.24 | −6.39E−01 | −1.30E+00 | 2 | Unchanged Medium |
| P41091 | 565.2535 | −6.33E−01 | −6.46E−01 | 2 | Unchanged Medium |
| Q99613 | 997.2011 | −1.91E−01 | −4.19E−01 | 2 | Unchanged Medium |
| P78344 | 1419.665 | −4.08E−01 | −4.19E−02 | 2 | Unchanged Medium |
| P23588 | 1160.659 | −2.54E−01 | −4.07E−01 | 2 | Unchanged Medium |
| Q13541 | 1669.873 | 4.70E−01 | −6.43E−01 | 2 | Unchanged Medium |
| Q13542 | 1712.605 | 3.79E−02 | 1.95E−01 | 2 | Unchanged Medium |
| O60573 | 833.9147 | 3.49E−02 | 1.42E−01 | 2 | Unchanged Medium |
| Q16394 | 584.9471 | 6.63E−01 | −2.64E−01 | 2 | Unchanged Medium |
| P37268 | 610.2023 | −1.62E−01 | −6.73E−01 | 2 | Unchanged Medium |
| P55899 | 566.0193 | 7.86E−02 | 7.74E−01 | 2 | Unchanged Medium |
| Q9UK73 | 541.9489 | −4.67E−02 | −3.93E−03 | 2 | Unchanged Medium |
| P21333 | 966.2982 | 4.33E−01 | −4.86E−01 | 2 | Unchanged Medium |
| P26885 | 662.0387 | −3.01E−01 | −6.76E−01 | 2 | Unchanged Medium |
| Q13451 | 1097.248 | −7.50E−01 | −2.98E−01 | 2 | Unchanged Medium |
| O95633 | 1227.557 | −2.64E−01 | −3.86E−02 | 2 | Unchanged Medium |
| P15407 | 1421.01 | −4.77E−01 | −3.31E−01 | 2 | Unchanged Medium |
| Q14192 | 703.7424 | 1.97E+00 | 9.30E−01 | 2 | Unchanged Medium |
| Q9NZA0 | 981.791 | 2.65E−01 | −2.13E−02 | 2 | Unchanged Medium |
| P35637 | 893.3381 | 4.21E−01 | −4.06E−01 | 2 | Unchanged Medium |
| | 935.6666 | −2.32E−01 | 1.71E−01 | 2 | Unchanged Medium |
| Q9Y653 | 1889.081 | −4.21E−01 | 1.00E−01 | 2 | Unchanged Medium |
| Q9H1C0 | 727.2869 | 1.55E−02 | 7.47E−02 | 2 | Unchanged Medium |
| P15170 | 1521.226 | −8.80E−01 | −5.33E−01 | 2 | Unchanged Medium |
| O95166 | 1572.36 | −6.11E−02 | 7.87E−01 | 2 | Unchanged Medium |
| O08765 | 1095.839 | −4.34E−01 | −1.39E−01 | 2 | Unchanged Medium |
| P16278 | 665.3025 | 2.82E−01 | 5.69E−01 | 2 | Unchanged Medium |
| P78537 | 790.0409 | 1.77E−01 | −3.52E−01 | 2 | Unchanged Medium |
| P31150 | 722.7008 | 1.47E−02 | 4.97E−01 | 2 | Unchanged Medium |
| P06396 | 513.4286 | 9.40E−01 | 8.05E−01 | 2 | Unchanged Medium |
| P52657 | 641.1721 | 3.78E−01 | 3.46E−01 | 2 | Unchanged Medium |
| Q00403 | 582.2035 | −1.38E−01 | 6.61E−01 | 2 | Unchanged Medium |
| P48060 | 707.2232 | 1.54E−01 | 3.35E−02 | 2 | Unchanged Medium |
| P06744 | 726.7688 | −3.11E−01 | −7.05E−01 | 2 | Unchanged Medium |
| P30101 | 1726.668 | 3.78E−01 | −4.50E−01 | 2 | Unchanged Medium |
| O43836 | 1758.853 | −6.36E−02 | 1.29E−01 | 2 | Unchanged Medium |
| P15104 | 1130.151 | 1.17E−02 | −1.71E−01 | 2 | Unchanged Medium |
| Q06210 | 777.6585 | −1.06E−02 | −5.34E−01 | 2 | Unchanged Medium |
| Q9Y3D4 | 960.4756 | 3.07E−01 | 8.32E−03 | 2 | Unchanged Medium |
| Q9Y2Q3 | 533.8957 | −1.02E+00 | −1.28E+00 | 2 | Unchanged Medium |
| P48637 | 522.9685 | −4.13E−01 | −6.00E−01 | 2 | Unchanged Medium |
| P78417 | 1937.888 | −1.36E−01 | 2.95E−02 | 2 | Unchanged Medium |
| P49840 | 1254.087 | 2.39E−01 | 4.63E−01 | 2 | Unchanged Medium |
| P13224 | 557.0977 | 4.23E−01 | 1.22E−01 | 2 | Unchanged Medium |
| Q9UBQ7 | 973.9926 | 7.50E−02 | 4.55E−02 | 2 | Unchanged Medium |
| Q9P2T1 | 882.4154 | 1.01E−01 | 4.87E−01 | 2 | Unchanged Medium |
| Q9H4A6 | 1148.94 | 2.35E−01 | 2.35E−01 | 2 | Unchanged Medium |
| P28799 | 1532.188 | −3.79E−01 | 1.16E−01 | 2 | Unchanged Medium |
| Q14393 | 595.5619 | 6.09E−01 | 2.17E−01 | 2 | Unchanged Medium |
| P29354 | 746.3732 | −1.22E−01 | 2.44E−01 | 2 | Unchanged Medium |
| Q14451 | 731.6029 | −1.61E−01 | 2.47E−01 | 2 | Unchanged Medium |
| Q9Y6G2 | 1093.42 | −4.04E−1 | −3.43E−01 | 2 | Unchanged Medium |
| P04899 | 1334.01 | 1.02E+00 | 6.94E−01 | 2 | Unchanged Medium |
| P11016 | 789.4586 | 1.41E−01 | 2.73E−02 | 2 | Unchanged Medium |
| Q13905 | 504.61 | 3.84E−01 | 4.71E−01 | 2 | Unchanged Medium |
| Q16774 | 1095.967 | 1.08E−02 | 4.72E−01 | 2 | Unchanged Medium |
| Q9Y649 | 1971.464 | 1.41E−01 | 5.39E−01 | 2 | Unchanged Medium |
| P07305 | 1797.448 | −2.77E−02 | 6.73E−02 | 2 | Unchanged Medium |
| O75367 | 786.0181 | −1.23E+00 | −3.48E−01 | 2 | Unchanged Medium |
| P17317 | 874.2269 | 1.41E+00 | −3.58E−01 | 2 | Unchanged Medium |
| P02278 | 707.973 | 2.22E−01 | 5.38E−01 | 2 | Unchanged Medium |
| Q9NS37 | 725.1835 | −5.11E−01 | −2.59E−01 | 2 | Unchanged Medium |
| O96004 | 646.3401 | −1.84E−01 | −2.04E−01 | 2 | Unchanged Medium |
| P10809 | 1330.235 | −4.08E−01 | −7.30E−01 | 2 | Unchanged Medium |
| P34932 | 1352.283 | −4.28E−01 | 2.01E−01 | 2 | Unchanged Medium |
| P07900 | 1783.374 | −6.70E−01 | −7.48E−01 | 2 | Unchanged Medium |
| P08238 | 1903.993 | −1.51E+00 | −5.11E−01 | 2 | Unchanged Medium |
| O75506 | 1171.256 | −6.57E−02 | 1.99E−01 | 2 | Unchanged Medium |
| Q9UHG4 | 549.5035 | 4.80E−01 | 6.73E−01 | 2 | Unchanged Medium |
| P09105 | 651.9692 | 1.31E−01 | −5.37E−01 | 2 | Unchanged Medium |
| O43504 | 1097.672 | −8.79E−02 | 4.05E−01 | 2 | Unchanged Medium |
| Q9H2I6 | 590.1783 | −5.14E−01 | −1.27E−01 | 2 | Unchanged Medium |
| P51858 | 850.7213 | −8.65E−01 | 3.85E−01 | 2 | Unchanged Medium |
| Q04150 | 959.787 | 1.76E−01 | −1.16E−01 | 2 | Unchanged Medium |
| Q13151 | 998.1703 | 3.34E−01 | 5.77E−02 | 2 | Unchanged Medium |
| P22626 | 1206.298 | 6.33E−01 | −1.38E−02 | 2 | Unchanged Medium |
| P07910 | 841.6601 | 5.03E−01 | −5.79E−02 | 2 | Unchanged Medium |
| O14979 | 1563.717 | 6.38E−01 | 9.10E−01 | 2 | Unchanged Medium |
| P52597 | 1567.536 | −3.27E−01 | −5.69E−01 | 2 | Unchanged Medium |
| Q07244 | 733.1055 | −3.09E−01 | −3.46E−01 | 2 | Unchanged Medium |
| Q15584 | 717.4409 | 2.43E−01 | −3.51E−01 | 2 | Unchanged Medium |
| P12081 | 514.8575 | −3.89E−01 | 2.34E−02 | 2 | Unchanged Medium |
| Q9UBN7 | 595.8457 | 2.01E−02 | −7.53E−01 | 2 | Unchanged Medium |
| Q13838 | 735.5784 | 4.36E−01 | −1.75E−01 | 2 | Unchanged Medium |
| Q99873 | 978.6584 | 4.43E−01 | −9.05E−02 | 2 | Unchanged Medium |
| Q9H063 | 893.139 | 4.45E−01 | 3.05E−01 | 2 | Unchanged Medium |
| P51610 | 632.9786 | 3.33E−01 | −9.25E−03 | 2 | Unchanged Medium |
| O00165 | 1800.489 | 7.11E−01 | 1.12E−02 | 2 | Unchanged Medium |
| Q9Y2Q5 | 752.165 | 1.13E−02 | 7.02E−01 | 2 | Unchanged Medium |
| Q9Y2R0 | 1668.909 | 2.44E−01 | 1.87E−01 | 2 | Unchanged Medium |
| Q9Y2T0 | 504.1821 | −4.73E−01 | 6.94E−01 | 2 | Unchanged Medium |
| Q9UNZ5 | 1159.538 | 7.07E−01 | 6.62E−01 | 2 | Unchanged Medium |
| Q9P032 | 668.1435 | 5.93E−01 | 1.08E−01 | 2 | Unchanged Medium |
| Q9P019 | 1312.254 | 1.08E+00 | 1.24E+00 | 2 | Unchanged Medium |
| Q9P004 | 932.7666 | −1.11E+00 | −1.43E−01 | 2 | Unchanged Medium |
| Q9P003 | 1118.827 | 5.19E−01 | 8.11E−01 | 2 | Unchanged Medium |
| Q9P000 | 650.7428 | 4.24E−02 | 8.57E−01 | 2 | Unchanged Medium |
| Q9Y684 | 665.044 | 4.85E−01 | 1.39E−01 | 2 | Unchanged Medium |
| Q9NRG2 | 1462.8 | 3.61E−01 | 2.07E−01 | 2 | Unchanged Medium |
| Q16775 | 531.4885 | −3.29E−01 | 4.03E−01 | 2 | Unchanged Medium |
| P40939 | 1733.575 | −1.66E−01 | −3.69E−02 | 2 | Unchanged Medium |
| P55084 | 882.3271 | 1.18E+00 | 7.22E−01 | 2 | Unchanged Medium |
| Q14526 | 943.6552 | 1.35E−01 | −2.30E−02 | 2 | Unchanged Medium |
| Q9UBS2 | 628.6277 | −3.79E−01 | 2.40E−02 | 2 | Unchanged Medium |
| Q96EW8 | 518.5858 | −1.70E+00 | −2.67E−01 | 2 | Unchanged Medium |
| Q9Y2S6 | 1423.596 | 4.23E−01 | 3.26E−01 | 2 | Unchanged Medium |
| Q9UKZ1 | 592.3978 | −3.77E−01 | −9.84E−02 | 2 | Unchanged Medium |
| Q9UJJ9 | 576.3525 | 4.75E−01 | 6.85E−01 | 2 | Unchanged Medium |
| O95891 | 1741.841 | −1.81E−01 | 3.07E−01 | 2 | Unchanged Medium |
| Q9UJI9 | 1019.924 | −1.55E−01 | −2.60E−01 | 2 | Unchanged Medium |
| Q9H0U3 | 1169.228 | −1.68E−01 | −2.59E−01 | 2 | Unchanged Medium |
| Q9NWD8 | 741.1753 | −8.68E−02 | 5.11E−03 | 2 | Unchanged Medium |
| Q9NW90 | 548.3816 | 2.89E−01 | 1.61E−01 | 2 | Unchanged Medium |
| Q9NW61 | 745.7235 | −3.14E−01 | 1.29E−01 | 2 | Unchanged Medium |
| Q9NW16 | 589.0741 | −6.93E−01 | −4.78E−01 | 2 | Unchanged Medium |
| Q9NVZ3 | 500.7932 | −2.70E−01 | 6.81E−01 | 2 | Unchanged Medium |
| Q9NVC3 | 976.6359 | −1.23E+00 | −5.95E−01 | 2 | Unchanged Medium |
| Q9H9K7 | 565.6072 | 4.12E−01 | 5.59E−01 | 2 | Unchanged Medium |
| Q9H8H4 | 1084.852 | 4.26E−01 | −4.50E−01 | 2 | Unchanged Medium |
| Q9P0R5 | 834.4521 | 6.70E−01 | −3.78E−02 | 2 | Unchanged Medium |
| Q9NXQ4 | 1521.653 | 3.89E−02 | 1.13E−02 | 2 | Unchanged Medium |
| Q9NXI2 | 566.3149 | −1.85E+00 | 4.47E−01 | 2 | Unchanged Medium |
| Q9NXD5 | 932.5386 | 2.97E−01 | 1.20E+00 | 2 | Unchanged Medium |
| Q9NX64 | 915.2399 | 6.42E−02 | −1.86E−01 | 2 | Unchanged Medium |
| Q9NWX1 | 1565.249 | 1.66E−01 | 3.95E−01 | 2 | Unchanged Medium |
| Q9NWT8 | 934.2965 | −1.12E−01 | −9.99E−02 | 2 | Unchanged Medium |
| Q9NWM3 | 554.671 | 8.56E−01 | 6.37E−01 | 2 | Unchanged Medium |
| Q9H7C7 | 615.0164 | 8.53E−02 | 3.61E−01 | 2 | Unchanged Medium |
| Q9H6R6 | 500.0538 | 6.75E−01 | −1.04E−01 | 2 | Unchanged Medium |
| Q9H673 | 1047.134 | −1.39E+00 | −1.98E−01 | 2 | Unchanged Medium |
| O95476 | 1279.962 | 1.81E−01 | −3.27E−01 | 2 | Unchanged Medium |
| Q9Y244 | 1481.658 | −4.23E−01 | −1.22E−01 | 2 | Unchanged Medium |
| Q9P022 | 610.0993 | −4.97E−02 | −3.56E−01 | 2 | Unchanged Medium |
| Q9P013 | 1167.03 | 3.06E−01 | 6.63E−01 | 2 | Unchanged Medium |
| Q9P009 | 1008.218 | −6.40E−01 | 3.67E−01 | 2 | Unchanged Medium |
| Q9P0S9 | 1622.151 | 5.51E−01 | 2.36E−01 | 2 | Unchanged Medium |
| Q9NPI0 | 532.0086 | 1.26E+00 | 1.20E+00 | 2 | Unchanged Medium |
| O95887 | 679.3596 | −4.95E−01 | −1.34E−01 | 2 | Unchanged Medium |
| Q9P0T9 | 1322.295 | 2.52E−01 | 7.04E−02 | 2 | Unchanged Medium |
| Q9P0Q1 | 774.623 | 1.43E+00 | 1.31E+00 | 2 | Unchanged Medium |
| Q9P0P0 | 1798.292 | 5.49E−01 | 7.50E−01 | 2 | Unchanged Medium |
| Q9P0N9 | 506.0883 | 1.61E−01 | −5.32E−01 | 2 | Unchanged Medium |
| Q9NZD9 | 607.2015 | −2.44E−01 | 2.52E−01 | 2 | Unchanged Medium |
| O75207 | 663.0017 | 3.63E−02 | −4.30E−02 | 2 | Unchanged Medium |
| Q9BWL0 | 579.5932 | 3.63E−01 | −3.87E−01 | 2 | Unchanged Medium |
| Q9BW61 | 1754.667 | −9.46E−02 | 7.68E−01 | 2 | Unchanged Medium |
| Q9BQ49 | 628.1009 | 3.59E−01 | 3.63E−01 | 2 | Unchanged Medium |
| Q9BQD3 | 876.8925 | 9.67E−02 | 7.95E−02 | 2 | Unchanged Medium |
| Q9BQ61 | 1794.728 | 6.82E−01 | 8.13E−01 | 2 | Unchanged Medium |
| Q9BTV4 | 548.9637 | 5.52E−02 | −1.63E−01 | 2 | Unchanged Medium |
| Q9BVX2 | 704.7101 | −3.06E−01 | 2.91E−02 | 2 | Unchanged Medium |
| Q9H773 | 734.1306 | −4.53E−01 | 3.57E−02 | 2 | Unchanged Medium |
| Q9BPX5 | 546.6359 | 1.16E−01 | −4.60E−01 | 2 | Unchanged Medium |
| Q9UJX8 | 1083.179 | −8.46E−02 | −3.21E−01 | 2 | Unchanged Medium |
| P00492 | 669.9473 | −1.10E+00 | −2.38E+00 | 2 | Unchanged Medium |
| P22304 | 1205.085 | −1.28E−01 | 1.43E−01 | 2 | Unchanged Medium |
| Q03827 | 574.4895 | −2.79E−01 | −2.78E−01 | 2 | Unchanged Medium |
| P12268 | 1038.44 | 4.39E−02 | −1.15E+00 | 2 | Unchanged Medium |
| Q9Y6K9 | 805.9933 | −2.60E−02 | −6.18E−02 | 2 | Unchanged Medium |
| Q14573 | 609.9521 | 1.75E−01 | 2.17E−01 | 2 | Unchanged Medium |
| O15357 | 1146.828 | 3.71E−01 | 2.08E−01 | 2 | Unchanged Medium |
| O15503 | 505.2037 | 3.84E−01 | −1.42E−01 | 2 | Unchanged Medium |
| P52945 | 1961.135 | −9.45E−01 | 1.36E+00 | 2 | Unchanged Medium |
| P51460 | 1184.891 | −2.38E−01 | 6.76E−02 | 2 | Unchanged Medium |
| P46977 | 1998.711 | 5.71E−01 | 7.81E−02 | 2 | Unchanged Medium |
| Q13418 | 1300.502 | 2.55E−01 | −4.79E−01 | 2 | Unchanged Medium |
| P48551 | 554.33 | −6.37E−01 | 1.44E−01 | 2 | Unchanged Medium |
| P15260 | 1648.504 | 1.64E−02 | 1.16E+00 | 2 | Unchanged Medium |
| P38484 | 638.9669 | −1.10E−01 | 3.80E−01 | 2 | Unchanged Medium |
| O14896 | 617.9736 | −2.85E−01 | 9.39E−01 | 2 | Unchanged Medium |
| | 515.9592 | −4.29E−01 | −3.80E−01 | 2 | Unchanged Medium |
| Q14116 | 1230.533 | −1.45E+00 | 7.71E−02 | 2 | Unchanged Medium |
| O75874 | 687.2716 | 4.53E−01 | −4.14E−01 | 2 | Unchanged Medium |
| P51553 | 832.7908 | −5.67E−02 | 1.40E−01 | 2 | Unchanged Medium |
| P41252 | 650.6946 | 6.24E−01 | −1.03E+00 | 2 | Unchanged Medium |
| Q9UE89 | 604.7722 | 8.21E−01 | −5.17E−01 | 2 | Unchanged Medium |
| P52292 | 1350.126 | 5.95E−01 | 2.04E−01 | 2 | Unchanged Medium |
| P24390 | 1392.342 | −1.41E−01 | −3.65E−01 | 2 | Unchanged Medium |
| P08779 | 754.7055 | −2.99E−01 | −5.39E−02 | 2 | Unchanged Medium |
| Q04695 | 937.9159 | 8.64E−01 | 1.02E+00 | 2 | Unchanged Medium |
| Q07666 | 1031.926 | −1.37E−01 | −6.37E−01 | 2 | Unchanged Medium |
| Q14165 | 669.3755 | 2.27E+00 | 2.89E−01 | 2 | Unchanged Medium |
| Q12765 | 979.0013 | 4.48E−02 | 7.68E−02 | 2 | Unchanged Medium |
| O43310 | 542.3144 | 2.06E−01 | 2.88E−01 | 2 | Unchanged Medium |
| O43167 | 1160.899 | −3.43E−01 | −5.10E−01 | 2 | Unchanged Medium |
| Q9H7D5 | 1291.483 | 2.60E−01 | 1.12E+00 | 2 | Unchanged Medium |
| Q9HD96 | 1044.592 | −3.76E−01 | −7.25E−01 | 2 | Unchanged Medium |
| O00182 | 864.4149 | −5.28E−01 | 7.79E−01 | 2 | Unchanged Medium |
| Q99538 | 695.3901 | 1.25E+00 | 1.53E−01 | 2 | Unchanged Medium |
| O95751 | 706.7407 | 1.50E−01 | −1.67E+00 | 2 | Unchanged Medium |
| O15468 | 962.0679 | 4.38E−01 | 1.84E−01 | 2 | Unchanged Medium |
| Q9UBB4 | 731.0054 | −3.41E−01 | −4.94E−01 | 2 | Unchanged Medium |
| Q9UFW4 | 1834.238 | 3.36E−01 | 1.79E−02 | 2 | Unchanged Medium |
| Q14847 | 772.769 | 4.30E−01 | 1.91E−01 | 2 | Unchanged Medium |
| O00158 | 703.0924 | 1.56E+00 | −6.16E−01 | 2 | Unchanged Medium |
| Q9HAP6 | 1945.862 | 4.10E−02 | −4.50E−01 | 2 | Unchanged Medium |
| P38571 | 568.1283 | −2.36E−01 | −4.02E−02 | 2 | Unchanged Medium |
| Q99732 | 674.31 | −5.98E−01 | 3.92E−01 | 2 | Unchanged Medium |
| Q9Y4Z1 | 1511.517 | −3.95E−02 | −3.40E−01 | 2 | Unchanged Medium |
| O75896 | 688.0098 | −2.00E−01 | 1.61E−01 | 2 | Unchanged Medium |
| Q06643 | 582.7217 | −4.41E−01 | 1.36E+00 | 2 | Unchanged Medium |
| O95372 | 656.485 | 6.49E−01 | 6.18E−01 | 2 | Unchanged Medium |
| P13473 | 1408.92 | 4.24E−01 | 1.07E+00 | 2 | Unchanged Medium |
| Q08397 | 1753.531 | 6.89E−01 | 2.25E−01 | 2 | Unchanged Medium |
| Q15046 | 665.5485 | −7.39E−01 | −6.96E−01 | 2 | Unchanged Medium |
| Q13312 | 628.231 | −4.92E−01 | −6.85E−01 | 2 | Unchanged Medium |
| Q09160 | 1841.106 | −2.40E−01 | 8.30E−01 | 2 | Unchanged Medium |
| P40925 | 1007.157 | −7.12E−02 | −5.38E−01 | 2 | Unchanged Medium |
| P40926 | 1251.158 | −3.32E−01 | −5.88E−01 | 2 | Unchanged Medium |
| Q16626 | 1536.52 | 5.44E−01 | 2.87E−01 | 2 | Unchanged Medium |
| Q9NQG1 | 689.9075 | 4.66E−01 | 5.85E−01 | 2 | Unchanged Medium |
| P26572 | 625.3732 | 5.18E−01 | −7.49E−01 | 2 | Unchanged Medium |
| Q10469 | 606.4146 | 8.52E−02 | −4.29E−02 | 2 | Unchanged Medium |
| P41223 | 503.5179 | 6.29E−01 | 5.96E−01 | 2 | Unchanged Medium |
| Q9Y5V3 | 1008.268 | 2.08E+00 | 1.26E+00 | 2 | Unchanged Medium |
| P43121 | 704.4837 | −1.04E+00 | −1.56E−01 | 2 | Unchanged Medium |
| Q92494 | 952.9144 | −2.23E−01 | 1.11E−01 | 2 | Unchanged Medium |
| Q14444 | 560.0583 | −2.41E−01 | 2.77E−01 | 2 | Unchanged Medium |
| Q9NPE2 | 922.5469 | 5.33E−01 | 3.14E−01 | 2 | Unchanged Medium |
| Q92571 | 533.4868 | −2.01E−01 | −4.71E−01 | 2 | Unchanged Medium |
| O95204 | 520.899 | −6.19E−01 | −1.08E+00 | 2 | Unchanged Medium |
| P80297 | 582.747 | 1.55E−01 | −2.32E−01 | 2 | Unchanged Medium |
| Q99735 | 529.1741 | −4.96E−01 | 3.79E−01 | 2 | Unchanged Medium |
| Q98XW5 | 609.0447 | 1.84E+00 | 1.49E−00 | 2 | Unchanged Medium |
| Q9Y6C9 | 1892.145 | 1.39E−02 | 2.56E−02 | 2 | Unchanged Medium |
| Q9Y3B7 | 517.9208 | 8.68E−01 | 6.88E−02 | 2 | Unchanged Medium |
| Q9P015 | 634.2498 | 8.94E−02 | 4.57E−01 | 2 | Unchanged Medium |
| Q9NX20 | 613.4293 | 1.25E−01 | −3.74E−01 | 2 | Unchanged Medium |
| Q9H0U6 | 856.3674 | 1.57E−01 | 4.32E−01 | 2 | Unchanged Medium |
| Q9NZE8 | 927.2731 | 6.45E−02 | −7.77E−03 | 2 | Unchanged Medium |
| Q9P0P3 | 1101.306 | −2.51E−02 | −4.50E−01 | 2 | Unchanged Medium |
| Q9Y317 | 1063.499 | 7.79E−01 | 3.49E−01 | 2 | Unchanged Medium |
| Q9Y6G3 | 690.2211 | 2.70E−01 | 5.02E−01 | 2 | Unchanged Medium |
| Q9BYC7 | 591.6118 | 3.36E−01 | 4.39E−01 | 2 | Unchanged Medium |
| Q9Y3D3 | 997.1495 | 5.88E−02 | 7.41E−02 | 2 | Unchanged Medium |
| Q9Y676 | 674.9976 | 1.57E−01 | −2.37E−01 | 2 | Unchanged Medium |
| Q9Y3D5 | 865.8021 | 2.46E−01 | 6.27E−01 | 2 | Unchanged Medium |
| P82921 | 1019.33 | 5.76E−01 | 6.75E−01 | 2 | Unchanged Medium |
| P82650 | 513.1921 | −2.36E−01 | −1.07E−01 | 2 | Unchanged Medium |
| Q9Y2Q9 | 1395.534 | −1.14E−01 | −2.15E−02 | 2 | Unchanged Medium |
| Q9NP92 | 606.105 | −1.16E−01 | −3.31E−01 | 2 | Unchanged Medium |
| Q02750 | 590.1129 | −4.40E−01 | 1.87E−01 | 2 | Unchanged Medium |
| Q02779 | 510.095 | −2.29E−01 | −4.96E−01 | 2 | Unchanged Medium |
| Q12851 | 994.1804 | −1.67E−02 | 4.26E−02 | 2 | Unchanged Medium |
| P49137 | 1004.624 | 1.81E−01 | 8.87E−02 | 2 | Unchanged Medium |
| Q9BYG3 | 502.0002 | −1.36E+00 | −9.61E−01 | 2 | Unchanged Medium |
| Q15014 | 1144.777 | 1.39E−01 | −1.61E−01 | 2 | Unchanged Medium |
| | 999.5166 | −2.17E−01 | −1.60E−02 | 2 | Unchanged Medium |
| Q15773 | 603.749 | 4.09E−01 | −3.06E−01 | 2 | Unchanged Medium |
| P35579 | 1261.007 | −2.44E−01 | −3.07E−01 | 2 | Unchanged Medium |
| Q9Y6D2 | 504.9319 | −9.51E−01 | 4.14E−01 | 2 | Unchanged Medium |
| P41227 | 841.2057 | 3.13E−02 | 1.76E−01 | 2 | Unchanged Medium |
| Q13510 | 1028.456 | −1.51E−01 | 1.14E+00 | 2 | Unchanged Medium |
| O43678 | 1810.699 | 6.45E−01 | 4.96E−01 | 2 | Unchanged Medium |
| O95167 | 1570.238 | 7.39E−02 | 3.08E−01 | 2 | Unchanged Medium |
| P56556 | 1120.973 | −1.05E+00 | −8.49E−01 | 2 | Unchanged Medium |
| O95182 | 750.6457 | −4.87E−02 | 5.03E−02 | 2 | Unchanged Medium |
| P51970 | 767.6928 | 8.82E−01 | 3.93E−01 | 2 | Unchanged Medium |
| O96000 | 1219.156 | 1.84E−03 | 3.71E−01 | 2 | Unchanged Medium |
| O95178 | 836.3224 | −1.83E−01 | −2.68E−01 | 2 | Unchanged Medium |
| O95139 | 1678.472 | 6.34E−01 | 8.78E−02 | 2 | Unchanged Medium |
| P17568 | 648.3703 | −4.13E−01 | −4.74E−01 | 2 | Unchanged Medium |
| Q9Y6M9 | 1164.524 | −7.99E−01 | 1.55E−01 | 2 | Unchanged Medium |
| O43677 | 573.2205 | −1.39E−01 | 3.43E−02 | 2 | Unchanged Medium |
| O00217 | 1234.615 | −2.12E−01 | −2.96E−01 | 2 | Unchanged Medium |
| P19404 | 650.0945 | −1.69E+00 | −1.38E+00 | 2 | Unchanged Medium |
| O76008 | 698.4474 | −7.64E−01 | 4.01E−01 | 2 | Unchanged Medium |
| Q9UPY4 | 505.6782 | 5.46E−01 | 1.40E−01 | 2 | Unchanged Medium |
| | 1042.081 | −2.79E−01 | −5.27E−01 | 2 | Unchanged Medium |
| Q99742 | 695.8521 | −8.28E−01 | −1.41E+00 | 2 | Unchanged Medium |
| O60448 | 1662.051 | −2.97E−01 | −1.38E−01 | 2 | Unchanged Medium |
| Q9UNW9 | 1106.982 | 2.16E−01 | 1.70E−03 | 2 | Unchanged Medium |
| Q9UGL9 | 923.3423 | −1.43E+00 | 4.08E−01 | 2 | Unchanged Medium |
| Q9UGL6 | 1086.502 | 6.53E−02 | 6.09E−01 | 2 | Unchanged Medium |
| Q92982 | 519.6952 | −1.53E−01 | 5.88E−01 | 2 | Unchanged Medium |
| Q9BPW8 | 640.3075 | −5.59E−01 | −6.50E−01 | 2 | Unchanged Medium |
| Q9NQR4 | 684.4701 | 3.59E−01 | 7.42E−01 | 2 | Unchanged Medium |
| Q9UF10 | 1608.811 | 1.55E−01 | 5.83E−01 | 2 | Unchanged Medium |
| Q9Y385 | 701.1992 | 9.84E−02 | 3.69E−01 | 2 | Unchanged Medium |
| Q15233 | 1174.503 | 1.88E−01 | 1.55E−01 | 2 | Unchanged Medium |
| Q13137 | 923.1712 | 5.26E−02 | 5.31E−03 | 2 | Unchanged Medium |
| Q16236 | 1153.088 | −2.41E−01 | −6.20E−02 | 2 | Unchanged Medium |
| P08651 | 669.8575 | −1.38E−01 | −2.19E−01 | 2 | Unchanged Medium |
| O95134 | 508.0938 | 2.06E−01 | 1.67E−01 | 2 | Unchanged Medium |
| Q15772 | 1184.738 | −3.49E−01 | −6.75E−01 | 2 | Unchanged Medium |
| Q9UHY1 | 694.4539 | 2.64E−01 | 8.77E−01 | 2 | Unchanged Medium |
| Q9UPC9 | 982.77 | 1.81E−01 | 5.36E−01 | 2 | Unchanged Medium |
| Q15325 | 1808.771 | 3.28E−01 | 6.25E−02 | 2 | Unchanged Medium |
| O00567 | 630.2325 | −8.14E−02 | −1.17E−01 | 2 | Unchanged Medium |
| Q9NX24 | 561.9547 | 3.51E−01 | 4.22E−01 | 2 | Unchanged Medium |
| P06748 | 1275.358 | −4.98E−01 | −5.67E−01 | 2 | Unchanged Medium |
| P50583 | 684.354 | −2.66E+00 | −2.91E+00 | 2 | Unchanged Medium |
| Q9UHM6 | 1305.239 | 6.10E−01 | 1.50E−01 | 2 | Unchanged Medium |
| Q9Y218 | 1302.823 | −1.54E−01 | 9.66E−01 | 2 | Unchanged Medium |
| Q16612 | 967.7934 | 2.79E+00 | 1.12E−01 | 2 | Unchanged Medium |
| P55771 | 548.4374 | −2.73E−01 | −8.67E−01 | 2 | Unchanged Medium |
| Q9UKJ1 | 560.9087 | −2.72E−01 | −5.83E−01 | 2 | Unchanged Medium |
| Q15165 | 604.4337 | 7.86E−01 | 1.40E+00 | 2 | Unchanged Medium |
| Q07002 | 795.1858 | −4.03E−01 | 1.31E−01 | 2 | Unchanged Medium |
| Q9Y3C6 | 602.0621 | −5.90E−02 | 4.14E−01 | 2 | Unchanged Medium |
| P30405 | 745.2396 | −8.50E−01 | 1.33E−01 | 2 | Unchanged Medium |
| O43924 | 567.4949 | 9.06E−01 | 7.56E−01 | 2 | Unchanged Medium |
| P17858 | 750.1835 | −2.95E−01 | −8.56E−01 | 2 | Unchanged Medium |
| Q01813 | 591.6879 | −4.50E−02 | 1.71E−01 | 2 | Unchanged Medium |
| O00511 | 1704.656 | 7.83E−01 | 1.70E−01 | 2 | Unchanged Medium |
| P11216 | 666.5476 | 9.35E−01 | −6.30E−02 | 2 | Unchanged Medium |
| P00749 | 1861.568 | −5.98E−02 | 1.65E+00 | 2 | Unchanged Medium |
| P13797 | 1062.438 | 4.93E−01 | −3.00E−01 | 2 | Unchanged Medium |
| Q15102 | 537.6672 | −3.08E−03 | 5.20E−01 | 2 | Unchanged Medium |
| P01127 | 543.5869 | −1.33E−01 | 4.28E−01 | 2 | Unchanged Medium |
| Q15795 | 612.22 | −1.57E−01 | 3.55E−01 | 2 | Unchanged Medium |
| O43660 | 827.0995 | 1.13E−01 | 3.27E−01 | 2 | Unchanged Medium |
| Q15155 | 525.8869 | −4.38E−01 | −6.51E−01 | 2 | Unchanged Medium |
| Q9H361 | 653.8286 | −9.52E−02 | −6.29E−01 | 2 | Unchanged Medium |
| Q15366 | 1857.745 | 1.26E−01 | −3.86E−01 | 2 | Unchanged Medium |
| P19388 | 593.4201 | 3.39E−01 | −6.12E−02 | 2 | Unchanged Medium |
| P52434 | 1211.761 | 3.50E−01 | 6.35E−01 | 2 | Unchanged Medium |
| P36954 | 1613.004 | −1.82E−01 | 1.84E−01 | 2 | Unchanged Medium |
| P53803 | 619.6184 | 2.70E−01 | 1.03E+00 | 2 | Unchanged Medium |
| Q9UQQ3 | 558.9966 | 1.98E−01 | 4.25E−01 | 2 | Unchanged Medium |
| Q13670 | 708.0473 | −6.11E−01 | −6.07E−02 | 2 | Unchanged Medium |
| Q9P0J7 | 1438.482 | −2.33E−01 | 2.97E−01 | 2 | Unchanged Medium |
| O60925 | 1737.289 | 1.89E−01 | 7.51E−02 | 2 | Unchanged Medium |
| Q9UHV9 | 743.3158 | 6.95E−01 | 7.78E−01 | 2 | Unchanged Medium |
| Q13519 | 638.4071 | 6.53E−01 | −2.83E−01 | 2 | Unchanged Medium |
| P04156 | 710.1438 | −1.85E−01 | −3.39E−01 | 2 | Unchanged Medium |
| Q9UI73 | 504.0018 | 3.45E−01 | 3.13E−01 | 2 | Unchanged Medium |
| Q9P0T3 | 1820.221 | 1.02E+00 | 3.83E−01 | 2 | Unchanged Medium |
| Q02809 | 991.2271 | 1.98E+00 | −7.97E−02 | 2 | Unchanged Medium |
| P07237 | 538.3489 | −1.39E−01 | −6.44E−01 | 2 | Unchanged Medium |
| P09466 | 567.0247 | 9.49E−01 | 7.68E−01 | 2 | Unchanged Medium |
| Q16342 | 661.3332 | −5.84E−01 | 4.52E−02 | 2 | Unchanged Medium |
| O75340 | 1167.782 | 3.62E−01 | 4.70E−02 | 2 | Unchanged Medium |
| P35232 | 1818.121 | −3.54E−01 | −1.78E−01 | 2 | Unchanged Medium |
| Q9UQ80 | 1763.59 | 7.44E−02 | −4.58E−01 | 2 | Unchanged Medium |
| Q12796 | 1643.691 | −3.85E−01 | −3.36E−01 | 2 | Unchanged Medium |
| Q15188 | 1232.034 | 4.93E−01 | 3.76E−01 | 2 | Unchanged Medium |
| P35998 | 1090.401 | 6.85E−02 | 2.49E−01 | 2 | Unchanged Medium |
| P47210 | 127.919 | 2.02E−01 | 1.53E−01 | 2 | Unchanged Medium |
| O00495 | 701.7628 | −8.89E−01 | −1.07E+00 | 2 | Unchanged Medium |
| O75831 | 630.7237 | −5.92E−01 | −2.97E−01 | 2 | Unchanged Medium |
| Q13200 | 523.3702 | −9.59E−01 | −1.16E−01 | 2 | Unchanged Medium |
| O43242 | 1675.386 | −3.99E−01 | −8.13E−01 | 2 | Unchanged Medium |
| P55036 | 591.0419 | 3.53E−01 | 6.51E−01 | 2 | Unchanged Medium |
| P48556 | 1317.42 | 4.42E−01 | −2.72E−01 | 2 | Unchanged Medium |
| Q92530 | 1724.253 | −3.24E−03 | −2.04E−01 | 2 | Unchanged Medium |
| P28066 | 769.7417 | 1.62E−02 | 1.46E−03 | 2 | Unchanged Medium |
| O14818 | 1308.162 | −1.24E−01 | 3.12E−01 | 2 | Unchanged Medium |
| P49721 | 1853.89 | −1.81E−01 | 2.04E−01 | 2 | Unchanged Medium |
| P28062 | 735.864 | −6.58E−01 | 1.82E−01 | 2 | Unchanged Medium |
| P28065 | 1133.626 | 3.79E−01 | 1.08E+00 | 2 | Unchanged Medium |
| P10619 | 1142.279 | −6.51E−01 | −7.62E−01 | 2 | Unchanged Medium |
| Q9UNN8 | 694.2014 | −8.69E−01 | −3.46E−01 | 2 | Unchanged Medium |
| Q15084 | 782.4339 | −3.32E−01 | −1.41E+00 | 2 | Unchanged Medium |
| Q9Y2B9 | 814.4047 | 2.13E−01 | 6.73E−01 | 2 | Unchanged Medium |
| Q13517 | 532.9442 | 7.27E−01 | 6.25E−01 | 2 | Unchanged Medium |
| P54619 | 632.0978 | 8.01E−01 | 2.45E−01 | 2 | Unchanged Medium |
| P17612 | 724.5168 | 4.87E−01 | −3.19E−02 | 2 | Unchanged Medium |
| P09131 | 584.2407 | 2.22E−01 | 1.98E−01 | 2 | Unchanged Medium |
| O60927 | 1630.486 | −2.99E−01 | 2.42E−02 | 2 | Unchanged Medium |
| P41236 | 578.9436 | 1.34E−01 | 2.23E−01 | 2 | Unchanged Medium |
| Q15435 | 784.0242 | 2.48E−01 | 6.01E−01 | 2 | Unchanged Medium |
| P05323 | 992.192 | −4.10E−01 | 8.39E−02 | 2 | Unchanged Medium |
| Q15257 | 996.8116 | 3.26E−01 | −3.29E−02 | 2 | Unchanged Medium |
| P33172 | 1357.256 | −7.51E−01 | −5.59E−01 | 2 | Unchanged Medium |
| O00743 | 936.2693 | −4.35E−01 | 1.02E−01 | 2 | Unchanged Medium |
| P38391 | 1175.447 | 4.45E−01 | −9.93E−04 | 2 | Unchanged Medium |
| P78324 | 657.1953 | −1.12E−01 | 2.13E−01 | 2 | Unchanged Medium |
| Q13332 | 1928.827 | 1.02E−01 | 1.57E−03 | 2 | Unchanged Medium |
| Q9HAD6 | 981.1143 | 1.46E−01 | −9.50E−02 | 2 | Unchanged Medium |
| O43445 | 631.8368 | −4.49E−01 | −5.01E−01 | 2 | Unchanged Medium |
| Q9Y6B3 | 794.9108 | 6.62E−01 | 1.04E−01 | 2 | Unchanged Medium |
| Q9HAN1 | 1019.233 | 3.14E−01 | 4.71E−01 | 2 | Unchanged Medium |
| Q9NQA4 | 727.7123 | −1.31E−01 | 3.70E−02 | 2 | Unchanged Medium |
| O43598 | 517.9666 | −4.41E−02 | 3.90E−01 | 2 | Unchanged Medium |
| O43257 | 1183.655 | 1.48E−01 | 9.40E−02 | 2 | Unchanged Medium |
| Q9NY06 | 693.4947 | −7.62E−01 | −8.29E−01 | 2 | Unchanged Medium |
| P17152 | 547.029 | 5.64E−01 | 7.00E−01 | 2 | Unchanged Medium |
| Q9Y6I9 | 533.7168 | 9.92E−01 | 3.79E−01 | 2 | Unchanged Medium |
| P54886 | 538.0842 | 1.16E+00 | −4.31E−01 | 2 | Unchanged Medium |
| Q13876 | 718.0657 | 1.45E−01 | −3.50E−01 | 2 | Unchanged Medium |
| O14679 | 839.1208 | −1.26E−01 | 7.02E−01 | 2 | Unchanged Medium |
| P53611 | 807.7755 | 3.08E−01 | −6.14E−02 | 2 | Unchanged Medium |
| P47224 | 693.8748 | −1.61E−01 | 4.11E−01 | 2 | Unchanged Medium |
| Q15907 | 1060.693 | 4.02E−01 | 1.53E−01 | 2 | Unchanged Medium |
| P51153 | 895.2783 | 8.70E−01 | 5.95E−01 | 2 | Unchanged Medium |
| P35287 | 571.2023 | 1.08E−01 | 1.02E−01 | 2 | Unchanged Medium |
| Q9NP72 | 629.3713 | 4.66E−01 | 7.89E−01 | 2 | Unchanged Medium |
| P11476 | 933.4801 | −6.51E−02 | −4.38E−01 | 2 | Unchanged Medium |
| Q9H0U4 | 638.0939 | 4.02E−01 | 3.90E−01 | 2 | Unchanged Medium |
| P08886 | 1858.384 | 5.65E−01 | 3.54E−01 | 2 | Unchanged Medium |
| O95716 | 701.181 | −7.08E−01 | −5.10E−01 | 2 | Unchanged Medium |
| P35239 | 566.5496 | −6.05E−01 | 2.42E−01 | 2 | Unchanged Medium |
| P20340 | 524.9788 | −2.72E−01 | 3.81E−01 | 2 | Unchanged Medium |
| P43487 | 1468.585 | 3.13E−01 | −2.84E−01 | 2 | Unchanged Medium |
| Q9UI26 | 1319.368 | −2.41E−01 | −3.52E−01 | 2 | Unchanged Medium |
| P47736 | 583.6391 | −9.25E−01 | −1.08E+00 | 2 | Unchanged Medium |
| Q15382 | 1754.23 | 1.88E−01 | 6.10E−01 | 2 | Unchanged Medium |
| P52199 | 816.6152 | −4.22E−01 | 1.50E+00 | 2 | Unchanged Medium |
| P35238 | 971.4616 | −4.48E−03 | −2.29E−01 | 2 | Unchanged Medium |
| Q14644 | 622.1475 | 2.58E−02 | 1.74E−02 | 2 | Unchanged Medium |
| Q15404 | 1925.544 | 5.86E−01 | 3.11E−01 | 2 | Unchanged Medium |
| Q13283 | 735.5498 | −5.68E−01 | −1.02E−01 | 2 | Unchanged Medium |
| Q9NVQ9 | 1286.027 | −9.24E−03 | −1.56E−01 | 2 | Unchanged Medium |
| O60895 | 663.6562 | 6.51E−01 | −1.43E−01 | 2 | Unchanged Medium |
| P35244 | 656.7974 | 7.65E−01 | −1.16E−01 | 2 | Unchanged Medium |
| Q99623 | 1020.486 | 1.14E−01 | −7.25E−01 | 2 | Unchanged Medium |
| Q9UMQ4 | 725.1999 | 6.36E−02 | −3.53E−01 | 2 | Unchanged Medium |
| Q9NQC3 | 991.1835 | −6.53E−01 | −1.32E+00 | 2 | Unchanged Medium |
| P47804 | 509.1489 | 6.38E−01 | 3.02E−01 | 2 | Unchanged Medium |
| Q16576 | 1250.347 | −9.46E−01 | −1.23E−01 | 2 | Unchanged Medium |
| O95357 | 532.4837 | −1.71E+00 | 7.14E−02 | 2 | Unchanged Medium |
| Q15299 | 1017.603 | −6.51E−02 | −1.15E−01 | 2 | Unchanged Medium |
| Q9UMY1 | 641.044 | −5.86E−02 | 2.51E−01 | 2 | Unchanged Medium |
| Q13017 | 627.5049 | −1.23E−01 | 5.27E−01 | 2 | Unchanged Medium |
| O43182 | 986.3979 | −4.89E−01 | −6.41E−01 | 2 | Unchanged Medium |
| O60274 | 531.8448 | −8.31E−01 | −1.76E−01 | 2 | Unchanged Medium |
| P07998 | 1976.991 | 2.99E−01 | 1.23E+00 | 2 | Unchanged Medium |
| P04843 | 1700.316 | 5.90E−01 | −1.37E−01 | 2 | Unchanged Medium |
| P04844 | 1540.461 | 2.56E−01 | −4.32E−01 | 2 | Unchanged Medium |
| P26373 | 1739.922 | −1.20E−01 | −7.97E−01 | 2 | Unchanged Medium |
| P40429 | 1694.794 | −8.21E−01 | 9.89E−02 | 2 | Unchanged Medium |
| P39019 | 1841.785 | 3.29E−01 | −2.81E−01 | 2 | Unchanged Medium |
| P78317 | 695.4611 | −1.77E−01 | −1.93E−01 | 2 | Unchanged Medium |
| Q99942 | 1030.595 | 1.47E−01 | −1.00E−01 | 2 | Unchanged Medium |
| O43148 | 564.3647 | −5.52E−01 | 3.50E−01 | 2 | Unchanged Medium |
| O02916 | 530.87 | −3.10E−01 | 2.36E−01 | 2 | Unchanged Medium |
| Q9Y580 | 545.5838 | 5.97E−01 | 9.45E−01 | 2 | Unchanged Medium |
| Q14621 | 581.142 | 2.08E−02 | 5.61E−01 | 2 | Unchanged Medium |
| Q93062 | 714.6837 | −4.00E−01 | −3.60E−01 | 2 | Unchanged Medium |
| Q99497 | 1958.111 | 4.21E−01 | −4.56E−01 | 2 | Unchanged Medium |
| Q99584 | 529.713 | 4.55E−01 | −2.25E−01 | 2 | Unchanged Medium |
| P23526 | 544.258 | −2.02E−01 | 1.32E−01 | 2 | Unchanged Medium |
| O43865 | 817.8431 | −1.65E+00 | −1.47E+00 | 2 | Unchanged Medium |
| O14826 | 534.3923 | −4.57E−01 | −5.24E−01 | 2 | Unchanged Medium |
| O75326 | 835.2276 | 2.46E−01 | −5.09E−02 | 2 | Unchanged Medium |
| O43278 | 1066.162 | −1.01E+00 | −3.74E−02 | 2 | Unchanged Medium |
| O00271 | 1332.552 | −6.55E−01 | 3.36E−01 | 2 | Unchanged Medium |
| Q9UEW8 | 1032.185 | −5.48E−01 | −2.63E−01 | 2 | Unchanged Medium |
| Q13043 | 781.5546 | 1.04E−02 | 1.58E−01 | 2 | Unchanged Medium |
| Q9Y282 | 1448.986 | 5.76E−01 | −1.41E−01 | 2 | Unchanged Medium |
| P35542 | 959.3499 | 1.05E−01 | 7.98E−01 | 2 | Unchanged Medium |
| O75790 | 779.5745 | 6.97E−02 | 3.16E−02 | 2 | Unchanged Medium |
| Q9P0V3 | 1483.624 | 3.54E−01 | 5.04E−01 | 2 | Unchanged Medium |
| Q99519 | 630.4428 | −5.15E−01 | −6.70E−02 | 2 | Unchanged Medium |
| Q92185 | 520.9105 | −4.28E−01 | −3.82E−01 | 2 | Unchanged Medium |
| Q9Y6A9 | 692.8575 | 9.83E−01 | 7.24E−01 | 2 | Unchanged Medium |
| P21378 | 626.0198 | 2.16E−01 | −2.91E−01 | 2 | Unchanged Medium |
| O15302 | 899.9534 | −3.06E−01 | −3.47E−01 | 2 | Unchanged Medium |
| O76094 | 1286.822 | −5.12E−02 | −6.11E−01 | 2 | Unchanged Medium |
| Q9UNL2 | 1085.128 | 5.24E−01 | −1.35E+00 | 2 | Unchanged Medium |
| P40763 | 521.4351 | −9.48E−01 | −3.02E−01 | 2 | Unchanged Medium |
| Q92783 | 857.8322 | −1.90E−01 | 3.01E−01 | 2 | Unchanged Medium |
| Q9H723 | 846.2392 | 7.47E−01 | −5.00E−01 | 2 | Unchanged Medium |
| O15258 | 759.8427 | 3.93E−01 | −1.63E−01 | 2 | Unchanged Medium |
| O14834 | 786.0877 | 3.66E−01 | 1.03E−01 | 2 | Unchanged Medium |
| O43617 | 1650.871 | −2.85E−01 | −7.94E−03 | 2 | Unchanged Medium |
| Q04837 | 1064.292 | −2.89E−01 | −5.00E−01 | 2 | Unchanged Medium |
| P05455 | 841.9268 | 1.29E−01 | −1.93E−01 | 2 | Unchanged Medium |
| P43331 | 1073.82 | 1.86E−01 | 4.63E−01 | 2 | Unchanged Medium |
| P09012 | 514.5708 | 3.23E−01 | −8.22E−01 | 2 | Unchanged Medium |
| P08579 | 581.0279 | 4.15E−01 | 1.83E−01 | 2 | Unchanged Medium |
| P09234 | 1269.64 | 3.05E−01 | 4.62E−01 | 2 | Unchanged Medium |
| P08578 | 1819.876 | −6.41E−02 | −1.28E−01 | 2 | Unchanged Medium |
| Q15357 | 570.2015 | 6.35E−01 | 9.41E−01 | 2 | Unchanged Medium |
| P14678 | 1686.175 | −2.29E−01 | −6.65E−01 | 2 | Unchanged Medium |
| Q13487 | 616.6789 | 3.24E−01 | 4.03E−01 | 2 | Unchanged Medium |
| P35326 | 721.2318 | 1.53E+00 | 1.83E+00 | 2 | Unchanged Medium |
| P55854 | 804.107 | 2.82E−01 | −2.67E−01 | 2 | Unchanged Medium |
| P14648 | 1839.36 | 7.90E−02 | 5.18E−01 | 2 | Unchanged Medium |
| P55011 | 616.497 | −1.94E−01 | 3.23E−01 | 2 | Unchanged Medium |
| O95258 | 523.9535 | 2.79E−01 | 1.20E−01 | 2 | Unchanged Medium |
| O15431 | 1336.855 | 5.27E−01 | −2.77E−01 | 2 | Unchanged Medium |
| P04920 | 1624.765 | 7.81E−02 | −5.33E−02 | 2 | Unchanged Medium |
| Q01650 | 832.1088 | 3.10E−01 | −3.99E−01 | 2 | Unchanged Medium |
| Q07890 | 575.1528 | 5.14E−01 | 2.51E−01 | 2 | Unchanged Medium |
| O60749 | 688.5915 | 1.08E+00 | 1.35E+00 | 2 | Unchanged Medium |
| O60493 | 1180.399 | 2.27E−01 | 4.22E−01 | 2 | Unchanged Medium |
| Q9Y5X3 | 670.591 | 2.63E−02 | −6.32E−02 | 2 | Unchanged Medium |
| Q01082 | 545.8922 | −5.62E−01 | 2.49E−01 | 2 | Unchanged Medium |
| P52788 | 839.7693 | −2.17E−01 | −6.78E−02 | 2 | Unchanged Medium |
| P17947 | 1366.573 | 3.32E−01 | −1.90E−01 | 2 | Unchanged Medium |
| Q14818 | 609.3662 | 5.16E−01 | −1.34E−01 | 2 | Unchanged Medium |
| Q15427 | 530.9445 | 2.92E−01 | 4.71E−01 | 2 | Unchanged Medium |
| Q15815 | 614.261 | −4.20E−01 | −4.18E−01 | 2 | Unchanged Medium |
| Q01130 | 522.9409 | −1.80E−01 | −7.18E−02 | 2 | Unchanged Medium |
| P23152 | 1480.13 | −1.16E−01 | −7.94E−02 | 2 | Unchanged Medium |
| Q13503 | 670.5377 | 2.44E−02 | −3.13E−01 | 2 | Unchanged Medium |
| Q9P2R9 | 522.8318 | 5.51E−02 | 1.69E−01 | 2 | Unchanged Medium |
| Q9H6I2 | 819.674 | −1.16E−01 | 3.24E−01 | 2 | Unchanged Medium |
| Q9BT81 | 525.7297 | 1.46E−01 | 9.25E−02 | 2 | Unchanged Medium |
| O60526 | 596.1019 | −3.78E−01 | −3.93E−01 | 2 | Unchanged Medium |
| Q9Y6X1 | 1700.638 | 3.87E−01 | −4.68E−01 | 2 | Unchanged Medium |
| P31948 | 500.9139 | 2.47E−01 | 3.32E−01 | 2 | Unchanged Medium |
| P21912 | 785.3111 | 9.93E−02 | 3.63E−01 | 2 | Unchanged Medium |
| O14521 | 601.9355 | 9.66E−02 | −3.18E−01 | 2 | Unchanged Medium |
| P53597 | 582.7447 | −3.48E−01 | −5.22E−01 | 2 | Unchanged Medium |
| O95605 | 569.4163 | −8.42E−01 | −6.39E−01 | 2 | Unchanged Medium |
| Q16550 | 1081.744 | 2.85E−01 | 8.24E−01 | 2 | Unchanged Medium |
| Q15526 | 524.7026 | 3.18E−01 | 1.56E−01 | 2 | Unchanged Medium |
| O43539 | 1178.519 | 2.12E−01 | 2.85E−01 | 2 | Unchanged Medium |
| Q9UPX1 | 546.8885 | −6.98E−01 | −3.51E−01 | 2 | Unchanged Medium |
| O95721 | 849.1506 | −2.75E−01 | 3.10E−01 | 2 | Unchanged Medium |
| O43391 | 548.1569 | −7.23E−01 | 3.17E−02 | 2 | Unchanged Medium |
| Q15833 | 686.0183 | 3.47E−01 | −2.49E−01 | 2 | Unchanged Medium |
| Q92804 | 847.4936 | 2.84E−01 | −5.30E−01 | 2 | Unchanged Medium |
| Q9Y490 | 1625.187 | 8.18E−01 | −2.01E−01 | 2 | Unchanged Medium |
| O94797 | 614.3777 | −8.58E−01 | −1.01E+00 | 2 | Unchanged Medium |
| Q13311 | 1004.505 | 4.15E−02 | 4.00E−01 | 2 | Unchanged Medium |
| Q16650 | 1645.184 | 3.37E−01 | 3.15E−01 | 2 | Unchanged Medium |
| P51864 | 1690.282 | −4.93E−01 | −3.61E−01 | 2 | Unchanged Medium |
| P55061 | 1615.143 | 3.56E−01 | −2.10E−01 | 2 | Unchanged Medium |
| Q15569 | 780.661 | −2.42E−01 | −7.31E−02 | 2 | Unchanged Medium |
| O60636 | 515.2157 | −3.19E−01 | 5.85E−01 | 2 | Unchanged Medium |
| O60637 | 770.9441 | −9.09E−01 | −4.68E−01 | 2 | Unchanged Medium |
| O95857 | 578.9636 | −9.67E−01 | −5.58E−02 | 2 | Unchanged Medium |
| Q99614 | 744.2616 | −1.52E−01 | 3.35E−01 | 2 | Unchanged Medium |
| Q13118 | 575.4732 | 6.07E−01 | 4.31E−01 | 2 | Unchanged Medium |
| Q15583 | 1105.268 | 3.64E−01 | 5.42E−01 | 2 | Unchanged Medium |
| Q9H3N1 | 507.428 | 7.46E−01 | 5.60E−02 | 2 | Unchanged Medium |
| O43396 | 1693.179 | −2.34E−01 | −9.18E−02 | 2 | Unchanged Medium |
| P04216 | 1541.609 | 4.61E+00 | 8.10E−01 | 2 | Unchanged Medium |
| P12956 | 1353.001 | 2.51E−01 | 3.46E−01 | 2 | Unchanged Medium |
| P16035 | 542.4517 | 1.34E+00 | −2.54E−02 | 2 | Unchanged Medium |
| Q9H2X8 | 1033.648 | 9.65E−01 | 5.00E−01 | 2 | Unchanged Medium |
| Q13077 | 1097.404 | −7.42E−01 | 6.49E−03 | 2 | Unchanged Medium |
| Q9NZ34 | 1148.575 | 2.58E−01 | 1.69E−02 | 2 | Unchanged Medium |
| Q15369 | 854.8396 | −1.55E−01 | 5.11E−01 | 2 | Unchanged Medium |
| Q15906 | 608.5936 | 1.01E+00 | 2.25E−01 | 2 | Unchanged Medium |
| P50616 | 760.3803 | −7.78E−01 | −2.40E−02 | 2 | Unchanged Medium |
| Q13595 | 519.219 | 1.50E+00 | 1.31E+00 | 2 | Unchanged Medium |
| P37802 | 1798.473 | −7.60E−01 | 1.70E−01 | 2 | Unchanged Medium |
| Q9Y5Z9 | 1108.824 | −2.33E−01 | −5.97E−01 | 2 | Unchanged Medium |
| Q99595 | 521.2602 | −6.87E−03 | 5.60E−01 | 2 | Unchanged Medium |
| O60830 | 1744.175 | 2.75E−01 | −4.77E−02 | 2 | Unchanged Medium |
| O14925 | 737.451 | −1.91E−01 | 1.25E−01 | 2 | Unchanged Medium |
| Q9Y5J9 | 1615.991 | 1.06E−02 | −7.98E−02 | 2 | Unchanged Medium |
| Q15629 | 897.0752 | −6.84E−01 | 8.48E−02 | 2 | Unchanged Medium |
| Q99442 | 788.001 | 1.02E+00 | 5.51E−01 | 2 | Unchanged Medium |
| Q99805 | 1081.028 | −9.63E−02 | 3.03E−01 | 2 | Unchanged Medium |
| Q9Y2B0 | 995.6424 | 5.33E−01 | 9.54E−01 | 2 | Unchanged Medium |
| P49755 | 827.066 | −9.04E−01 | −1.48E+00 | 2 | Unchanged Medium |
| | 930.4505 | −9.59E−01 | −6.15E−01 | 2 | Unchanged Medium |
| Q03519 | 701.534 | −3.22E−01 | −5.72E−01 | 2 | Unchanged Medium |
| Q9NWF9 | 599.4053 | 4.83E−02 | −5.86E−01 | 2 | Unchanged Medium |
| Q15656 | 1792.501 | −1.54E−02 | −1.95E−01 | 2 | Unchanged Medium |
| Q92734 | 1624.429 | 1.50E−01 | 3.23E−01 | 2 | Unchanged Medium |
| Q12815 | 879.96 | −1.85E−01 | 4.15E−01 | 2 | Unchanged Medium |
| P09493 | 807.373 | 2.43E−02 | −1.20E+00 | 2 | Unchanged Medium |
| P49411 | 519.255 | −1.42E−01 | 7.90E−02 | 2 | Unchanged Medium |
| P05217 | 942.8088 | −3.87E−01 | −7.73E−01 | 2 | Unchanged Medium |
| P23258 | 1702.331 | 3.10E−01 | −5.03E−01 | 2 | Unchanged Medium |
| Q9UBN6 | 584.4772 | 1.75E−01 | 6.74E−01 | 2 | Unchanged Medium |
| Q13829 | 731.2933 | −6.20E−01 | −1.01E−01 | 2 | Unchanged Medium |
| O00496 | 661.0848 | 3.50E−01 | −1.82E−01 | 2 | Unchanged Medium |
| Q99816 | 705.4904 | −2.07E−01 | 8.08E−02 | 2 | Unchanged Medium |
| P16422 | 870.9367 | −1.95E+00 | −8.73E−01 | 2 | Unchanged Medium |
| Q9NP84 | 583.2792 | −3.04E−01 | −4.75E−01 | 2 | Unchanged Medium |
| Q9UJ47 | 807.3703 | 3.05E−02 | 4.69E−02 | 2 | Unchanged Medium |
| P31946 | 966.9847 | −7.52E−01 | −3.55E−01 | 2 | Unchanged Medium |
| P35214 | 850.9964 | −3.68E−01 | 1.67E−01 | 2 | Unchanged Medium |
| P27348 | 1615.078 | −9.91E−01 | −5.08E−01 | 2 | Unchanged Medium |
| O60704 | 547.7036 | 5.96E−02 | −1.14E+00 | 2 | Unchanged Medium |
| Q9Y4Z0 | 813.8478 | 1.28E−01 | −2.81E−01 | 2 | Unchanged Medium |
| P14927 | 922.1475 | −6.04E−02 | −2.85E−01 | 2 | Unchanged Medium |
| Q9UI02 | 1320.75 | 6.97E−01 | 6.16E−01 | 2 | Unchanged Medium |
| P31930 | 794.0747 | −3.01E−01 | 3.33E−03 | 2 | Unchanged Medium |
| P22695 | 562.0633 | 3.12E−01 | 4.40E−01 | 2 | Unchanged Medium |
| P07919 | 1382.391 | −4.02E−01 | 4.49E−01 | 2 | Unchanged Medium |
| P47985 | 1306.086 | −4.31E−01 | −2.58E−01 | 2 | Unchanged Medium |
| Q9NZ09 | 1598.619 | −1.90E−01 | 1.93E−01 | 2 | Unchanged Medium |
| Q9UNP0 | 538.3523 | 1.86E−01 | −9.48E−02 | 2 | Unchanged Medium |
| Q9UHP3 | 613.0845 | 1.94E−02 | 3.85E−01 | 2 | Unchanged Medium |
| P22314 | 1788.503 | 4.71E−03 | −1.75E−01 | 2 | Unchanged Medium |
| Q13404 | 1138.559 | 7.28E−02 | 1.77E−01 | 2 | Unchanged Medium |
| P51669 | 1247.427 | −1.09E−01 | 4.05E−02 | 2 | Unchanged Medium |
| P47986 | 567.8765 | −1.19E−01 | −2.47E−01 | 2 | Unchanged Medium |
| O14933 | 735.6835 | −2.66E−01 | 3.16E−01 | 2 | Unchanged Medium |
| Q16781 | 757.964 | −7.91E−01 | −1.78E−01 | 2 | Unchanged Medium |
| Q93068 | 1399.48 | −8.84E−02 | 1.51E−01 | 2 | Unchanged Medium |
| Q15386 | 596.8686 | 2.40E−01 | 2.45E−01 | 2 | Unchanged Medium |
| Q9UBK9 | 1125.743 | 5.65E−01 | 1.74E−01 | 2 | Unchanged Medium |
| O60512 | 529.5839 | 3.12E−01 | 4.81E−01 | 2 | Unchanged Medium |
| P78383 | 1816.963 | 4.74E−01 | 1.15E−01 | 2 | Unchanged Medium |
| Q16851 | 1302.468 | −5.79E−03 | −6.64E−02 | 2 | Unchanged Medium |
| Q9NZ45 | 672.9895 | 9.52E−02 | 3.98E−02 | 2 | Unchanged Medium |
| Q9NZ32 | 1214.412 | 3.87E−01 | 2.75E−01 | 2 | Unchanged Medium |
| Q9NZ29 | 923.518 | 7.62E−01 | 5.89E−01 | 2 | Unchanged Medium |
| Q15853 | 644.9023 | 2.14E−01 | 7.78E−02 | 2 | Unchanged Medium |
| Q92528 | 623.0076 | 1.54E−01 | 2.90E−01 | 2 | Unchanged Medium |
| P15692 | 619.202 | 8.95E−01 | 2.27E−01 | 2 | Unchanged Medium |
| P50552 | 502.9122 | −2.55E−01 | 4.67E−01 | 2 | Unchanged Medium |
| O60763 | 642.7624 | 2.21E−01 | −2.35E−01 | 2 | Unchanged Medium |
| Q15836 | 1076.578 | 3.99E−01 | 7.63E−01 | 2 | Unchanged Medium |
| Q9UEU0 | 621.1953 | 5.76E−01 | 5.72E−01 | 2 | Unchanged Medium |
| P14921 | 656.6528 | −1.65E+00 | −2.57E−01 | 2 | Unchanged Medium |
| P18206 | 767.5496 | 5.77E−01 | 5.62E−01 | 2 | Unchanged Medium |
| P00540 | 835.5051 | −7.49E−01 | −6.16E−01 | 2 | Unchanged Medium |
| P01106 | 514.7639 | −4.09E−01 | 1.61E−01 | 2 | Unchanged Medium |
| P21796 | 525.9036 | 2.72E−01 | 7.47E−02 | 2 | Unchanged Medium |
| Q15765 | 762.0788 | 3.70E−01 | 3.20E−02 | 2 | Unchanged Medium |
| P10398 | 744.4583 | −9.96E−01 | −7.32E−01 | 2 | Unchanged Medium |
| P11233 | 995.8044 | 3.50E−01 | 3.17E−01 | 2 | Unchanged Medium |
| Q9Y6W5 | 1468.514 | 2.19E−01 | 3.45E−01 | 2 | Unchanged Medium |
| Q9UPY6 | 1623.108 | 5.17E−01 | −3.09E−01 | 2 | Unchanged Medium |
| O75083 | 944.1492 | 1.10E−01 | 1.30E−01 | 2 | Unchanged Medium |
| O76071 | 746.1814 | 2.60E−01 | 5.58E−01 | 2 | Unchanged Medium |
| Q9HCN4 | 923.9753 | −1.24E+00 | −1.71E−01 | 2 | Unchanged Medium |
| Q9UM05 | 1672.59 | −2.91E−01 | −2.50E−01 | 2 | Unchanged Medium |
| Q9UQR1 | 604.9201 | 1.34E−01 | 5.21E−01 | 2 | Unchanged Medium |
| Q14119 | 1387.253 | −2.67E−03 | 5.57E−01 | 2 | Unchanged Medium |
| Q9NW07 | 554.3431 | 7.65E−01 | 5.15E−01 | 2 | Unchanged Medium |
| P20694 | 969.9823 | −2.84E−01 | −4.49E−02 | 2 | Unchanged Medium |

1-10. (canceled)

11. An isolated insulin-producing cell generated by a method comprising the step of culturing an IP cell in a cell culture medium comprising an effective amount of at least one differentiation promoting factor selected from the group consisting of C-Natriuretic Peptide (CNP), Calcitonin Gene Related Peptide, Cholera Toxin B Subunit, Dexamethasone, Gastrin-Releasing Peptide, Laminin, Met-Enkephalin, PDGFAA+PDGFBB, Sonic Hedgehog, and Substance P, such that the IP cell is transformed into an insulin-producing cell.

12. An insulin-producing cell, prepared by differentiating a mammalian acinar cell in vitro, wherein said insulin-producing cell has an expression profile after 16 days ex vivo as shown in Table 6.

13-20. (canceled)
